SEQUENCE LISTING (268 sequences)

SEQ ID NO: 1 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 2 - DNA, 1353 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 3 - DNA, 639 bp, Artificial Sequence
  Description of Artificial Sequence Synthetic polynucleotide
gacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 4 - DNA, 1353 bp, Artificial Sequence
  Description of Artificial Sequence Synthetic polynucleotide
caggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 5 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 213 aa, Artificial Sequence
  Description of Artificial Sequence Synthetic polypeptide
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 449 aa, Artificial Sequence
  Description of Artificial Sequence Synthetic polypeptide
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 774 bp, Artificial Sequence
  Description of Artificial Sequence Synthetic polynucleotide
cacggagaaggaacatttaccagcgacctcagcaagcagatggaggaagaggccgtgaggctgttcatcgagtggctgaagaacggcggaccctcctctggcgctccaccccctagcgggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 10 - DNA, 1911 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtattggcggaagcggagcaaagctcgccgcactgaaagccaagctggccgctctgaaggggggtggcggaagcgttccaattcaaaaggttcaagatgataccaaaactctgattaaaactattgtcacgcgtataaacgacatctcacatacccagtcggttagctcaaagcaaaaagttaccggtttggactttattccgggactgcacccgatcctgacccttagtaaaatggaccagacactggccgtctaccagcaaatcctgacatcgatgccatccagaaatgtgatacaaattagcaacgatttggaaaaccttcgcgatctgctgcacgtgctggccttcagtaagtcctgtcatctgccgtgggcgtcgggactggagactcttgactcgctgggtggagtgttagaggcctctggctattctactgaagtcgttgcgctgtcacgcctccaggggagcctgcaggacatgctgtggcagctggacctgtcacctggctgcggcggaggtgggagtgaactggccgcactggaagctgagctggctgccctcgaagctggaggctctggaacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 11 - DNA, 1902 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagaggcggaagcggagcaaagctcgccgcactgaaagccaagctggccgctctgaagggaggtggcgggagcgttccaattcaaaaggttcaagatgataccaaaactctgattaaaactattgtcacgcgtataaacgacatctcacatacccagtcggttagctcaaagcaaaaagttaccggtttggactttattccgggactgcacccgatcctgacccttagtaaaatggaccagacactggccgtctaccagcaaatcctgacatcgatgccatccagaaatgtgatacaaattagcaacgatttggaaaaccttcgcgatctgctgcacgtgctggccttcagtaagtcctgtcatctgccgtgggcgtcgggactggagactcttgactcgctgggtggagtgttagaggcctctggctattctactgaagtcgttgcgctgtcacgcctccaggggagcctgcaggacatgctgtggcagctggacctgtcacctggctgcggcggaggtgggagtgaactggccgcactggaagctgagctggctgccctcgaagctggaggctctggagactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 12 - DNA, 744 bp, Artificial Sequence
  Description of Artificial Sequence Synthetic polynucleotide
cacagccagggcacattcactagcgattatagtaaatatctggattccaaggcagcgcacgattttgtagagtggctcttgcgggccgggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 13 - DNA, 774 bp, Artificial Sequence
  Description of Artificial Sequence Synthetic polynucleotide
cacagccagggcacattcactagcgattatagtaaatatctggattccaaggcagcgcacgattttgtagagtggctcttgaacggaggcccttcctccggagctccacctccgtccgggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 14 - DNA, 774 bp, Artificial Sequence
  Description of Artificial Sequence Synthetic polynucleotide
cacagccagggcacattcactagcgattatagtaaatatctggattccaaggcagcgcacgattttgtagagtggctcttgaacggaggcccttcctccggagctccacctccgtccgggggtggcggaggcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 15 - DNA, 771 bp, Artificial Sequence
  Description of Artificial Sequence Synthetic polynucleotide
cacagccagggcacattcactagcgattatagtaaatatctggattccaaggcagcgcacgattttgtagagtggctcttgaacggaggcccttcctccggagctccacctccgtccgggggtggcggaagcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 16 - DNA, 771 bp, Artificial Sequence
  Description of Artificial Sequence Synthetic polynucleotide
cacagccagggcacattcactagcgattatagtaaatatctggattccaaggcagcgcacgattttgtagagtggctcttgaacggaggcccttcctccggagctccacctccgtccgggggtggcggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 17 - DNA, 1485 bp, Artificial Sequence
  Description of Artificial Sequence Synthetic polynucleotide
cacagccagggcacattcactagcgattatagtaaatatctggattccaaggcagcgcacgattttgtagagtggctcttgaacggaggcccttcctccggagctccacctccgtccgggggtggcggaagccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 18 - DNA, 1140 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgttctctgtctcaggaagacgctccgcagaccccgcgtccggttgctgaaatcgttccgtctttcatcaacaaagacaccgaaaccatcaacatgatgtctgaattcgttgctaacctgccgcaggaactgaaactgaccctgtctgaaatgcagccggctctgccgcagctgcagcagcacgttccggttctgaaagactcttctctgctgttcgaagaattcaaaaaactgatccgtaaccgtcagtctgaagctgctgactcttctccgtctgaactgaaatacctgggtctggacacccactctcgtaaaaaacgtcagctgtactctgctctggctaacaaatgctgccacgttggttgcaccaaacgttctctggctcgtttctgcggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 19 - DNA, 1212 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagcggcacttctgagtctgctactccagaaagcggcccaggttctgaaccagcaacttctggctctgagactccaggcacttctgagtccgcaacgcctgaatccggtcctggttctgaaccagctacttccggcagcgaaaccccaggtaccggaggtggcgggagccaccatcaccaccaccacggaggtggcgggagctctgagtctgcgactccagagtctggtcctggtacttccactgagcctagcgagggttccgcaccaggttctccggctggtagcccgaccagcacggaggagggtacgtctgaatctgcaacgccggaatcgggcccaggttcggagggaggaggtggcgggagccgtaaaaaacgtcagctgtactctgctctggctaacaaatgctgccacgttggttgcaccaaacgttctctggctcgtttctgcggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 20 - DNA, 1017 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgcggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 21 - DNA, 1017 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgcggcggaggtgggggtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 22 - DNA, 1050 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgcaacggaggcccttcctccggagctccacctccgtccggcggaggtgggggtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 23 - DNA, 1014 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgcggcggaggtgggagtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 24 - DNA, 1014 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgcggcggaggtgggggtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 25 - DNA, 1050 bp, Artificial Sequence; identical to SEQ ID NO: 22
  Description of Artificial Sequence Synthetic polynucleotide

SEQ ID NO: 26 - DNA, 921 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 27 - DNA, 921 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggcggaggtgggggtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 28 - DNA, 954 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcaacggaggcccttcctccggagctccacctccgtccggcggaggtgggggtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 29 - DNA, 918 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggcggaggtgggagtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 30 - DNA, 918 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggcggaggtgggggtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 31 - DNA, 1007 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggaggcccttcctccggagctccacctccgtccggcggaggtgggggtgacatccagatgacccagtccccctccaccctgtccgcctccgtgggcgaccgcgtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 32 - DNA, 921 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctcgtcgtgaagctgaagacctgcaggttggtcaggttgaactgggtggtggtccgggtgctggttctctgcagccgctggctctggaaggttctctgcagaaacgtcagctgtactctgctctggctaacaaatgctgccacgttggttgcaccaaacgttctctggctcgtttctgcggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 33 - DNA, 879 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctcagctgtactctgctctggctaacaaatgctgccacgttggttgcaccaaacgttctctggctcgtttctgcggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 34 - DNA, 828 bp, Artificial Sequence
  Description of Artificial Sequence Synthetic polynucleotide
tttgtgaaccaacacctgtgcggctcagacctggtggaagctctctacctagtgtgcggggaacgaggcttcttctacacagaccccaccggcggagggccccgccggggcattgtggaacaatgctgtcacagcatctgctccctctaccagctggagaactactgcaacggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 35 - DNA, 768 bp, Artificial Sequence
  Description of Artificial Sequence Synthetic polynucleotide
catagccagggaaccttcacctccgactacagcaaataccttgacagtaggagagctcaggattttgtgcaatggctgatgaacacaaagaggaataaaaacaatatagccgggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 36 - DNA, 753 bp, Artificial Sequence
  Description of Artificial Sequence Synthetic polynucleotide
cacggcgacggttcattctctgacgaaatgaatacaatactcgacaacctcgccgccagggactttatcaattggctcattcaaactaaaatcaccgacgggggtggcggaagcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 37 - DNA, 786 bp, Artificial Sequence
  Description of Artificial Sequence Synthetic polynucleotide
cacggcgacggttcattctctgacgaaatgaatacaatactcgacaacctcgccgccagggactttatcaattggctcattcaaactaaaatcaccgacggaggcccttcctccggagctccacctccgtccgggggtggcggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 38 - DNA, 759 bp, Artificial Sequence
  Description of Artificial Sequence Synthetic polynucleotide
atcaacgtgaagtgcagcctgccccagcagtgcatcaagccctgcaaggacgccggcatgcggttcggcaagtgcatgaacaagaagtgcaggtgctacagcgggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 39 - DNA, 795 bp, Artificial Sequence
  Description of Artificial Sequence Synthetic polynucleotide
gctgacaacaaatgcgaaaactctctgcgtcgtgaaatcgcttgcggtcagtgccgtgacaaagttaaaaccgacggttacttctacgaatgctgcacctctgactctaccttcaaaaaatgccaggacctgctgcacggcggaggtgggagtgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 40 - DNA, 756 bp, Artificial Sequence
  Description of Artificial Sequence Synthetic polynucleotide
gaatgcatcggtatgttcaaatcttgcgacccggaaaacgacaaatgctgcaaaggtcgtacctgctctcgtaaacaccgttggtgcaaatacaaactggggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 41 - DNA, 828 bp, Artificial Sequence
  Description of Artificial Sequence Synthetic polynucleotide
ctgaaatgttaccaacatggtaaagttgtgacttgtcatcgagatatgaagttttgctatcataacactggcatgccttttcgaaatctcaagctcatcctacagggatgttcttcttcgtgcagtgaaacagaaaacaataagtgttgctcaacagacagatgcaacaaagggggtggcggaagcgacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 42 - protein, 258 aa, Artificial Sequence
  Description of Artificial Sequence Synthetic polypeptide
HGEGTFTSDLSKQMEEEAVRLFIEWLKNGGPSSGAPPPSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 635 aa, Artificial Sequence
  Description of Artificial Sequence Synthetic polypeptide
EVQLVESGGGLVQPGGSLRLSCAASGFNIKDTYIHWVRQAPGKGLEWVARIGGSGAKLAALKAKLAALKGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSELAALEAELAALEAGGSGTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 632 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGGGGSELAALEAELAALEAGGSGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 248 aa, Artificial Sequence
  Description of Artificial Sequence Synthetic polypeptide
HSQGTFTSDYSKYLDSKAAHDFVEWLLRAGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 258 aa, Artificial Sequence
  Description of Artificial Sequence Synthetic polypeptide
HSQGTFTSDYSKYLDSKAAHDFVEWLLNGGPSSGAPPPSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 258 aa, Artificial Sequence
  Description of Artificial Sequence Synthetic polypeptide
HSQGTFTSDYSKYLDSKAAHDFVEWLLNGGPSSGAPPPSGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 257 aa, Artificial Sequence
  Description of Artificial Sequence Synthetic polypeptide
HSQGTFTSDYSKYLDSKAAHDFVEWLLNGGPSSGAPPPSGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 257 aa, Artificial Sequence
  Description of Artificial Sequence Synthetic polypeptide
HSQGTFTSDYSKYLDSKAAHDFVEWLLNGGPSSGAPPPSGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 493 aa, Artificial Sequence
  Description of Artificial Sequence Synthetic polypeptide
HSQGTFTSDYSKYLDSKAAHDFVEWLLNGGPSSGAPPPSGGGGSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 380 aa, Artificial Sequence
  Description of Artificial Sequence Synthetic polypeptide
DSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFCGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 404 aa, Artificial Sequence
  Description of Artificial Sequence Synthetic polypeptide
DSWMEEVIKLCGRELVRAQIAICGMSTWSKRGGGGSGTSESATPESGPGSEPATSGSETPGTSESATPESGPGSEPATSGSETPGTGGGGSHHHHHHGGGGSSESATPESGPGTSTEPSEGSAPGSPAGSPTSTEEGTSESATPESGPGSEGGGGGSRKKRQLYSALANKCCHVGCTKRSLARFCGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 339 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFCGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 339 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFCGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 350 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFCNGGPSSGAPPPSGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 338 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFCGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 338 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFCGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 350 aa, Artificial Sequence; identical to SEQ ID NO: 55
  Description of Artificial Sequence Synthetic polypeptide

SEQ ID NO: 59 - protein, 307 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 307 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGGGGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 317 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGPSSGAPPPSGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 306 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 306 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 317 aa, Artificial Sequence; identical to SEQ ID NO: 61
  Description of Artificial Sequence Synthetic polypeptide

SEQ ID NO: 65 - protein, 307 aa, Artificial Sequence
  Description of Artificial Sequence Synthetic polypeptide
DSWMEEVIKLCGRELVRAQIAICGMSTWSRREAEDLQVGQVELGGGPGAGSLQPLALEGSLQKRQLYSALANKCCHVGCTKRSLARFCGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 293 aa, Artificial Sequence
  Description of Artificial Sequence Synthetic polypeptide
DSWMEEVIKLCGRELVRAQIAICGMSTWSSGSETPGTSESATPESGPGSPQLYSALANKCCHVGCTKRSLARFCGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 276 aa, Artificial Sequence
  Description of Artificial Sequence Synthetic polypeptide
FVNQHLCGSDLVEALYLVCGERGFFYTDPTGGGPRRGIVEQCCHSICSLYQLENYCNGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 256 aa, Artificial Sequence
  Description of Artificial Sequence Synthetic polypeptide
HSQGTFTSDYSKYLDSRRAQDFVQWLMNTKRNKNNIAGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 251 aa, Artificial Sequence
  Description of Artificial Sequence Synthetic polypeptide
HGDGSFSDEMNTILDNLAARDFINWLIQTKITDGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 262 aa, Artificial Sequence
  Description of Artificial Sequence Synthetic polypeptide
HGDGSFSDEMNTILDNLAARDFINWLIQTKITDGGPSSGAPPPSGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 253 aa, Artificial Sequence
  Description of Artificial Sequence Synthetic polypeptide
INVKCSLPQQCIKPCKDAGMRFGKCMNKKCRCYSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 264 aa, Artificial Sequence
  Description of Artificial Sequence Synthetic polypeptide
ADNKCENSLRREIACGQCRDKVKTDGYFYECCTSDSTFKKCQDLLHGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 251 aa, Artificial Sequence
  Description of Artificial Sequence Synthetic polypeptide
ECIGMFKSCDPENDKCCKGRTCSRKHRWCKYKLGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 274 aa, Artificial Sequence
  Description of Artificial Sequence Synthetic polypeptide
LKCYQHGKVVTCHRDMKFCYHNTGMPFRNLKLILQGCSSSCSETENNKCCSTDRCNKGGGGSIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 117 bp, Artificial Sequence
  Description of Artificial Sequence Synthetic polynucleotide
cacggagaaggaacatttaccagcgacctcagcaagcagatggaggaagaggccgtgaggctgttcatcgagtggctgaagaacggcggaccctcctctggcgctccaccccctagc

SEQ ID NO: 76 - DNA, 438 bp, Artificial Sequence
  Description of Artificial Sequence Synthetic polynucleotide
gttccaattcaaaaggttcaagatgataccaaaactctgattaaaactattgtcacgcgtataaacgacatctcacatacccagtcggttagctcaaagcaaaaagttaccggtttggactttattccgggactgcacccgatcctgacccttagtaaaatggaccagacactggccgtctaccagcaaatcctgacatcgatgccatccagaaatgtgatacaaattagcaacgatttggaaaaccttcgcgatctgctgcacgtgctggccttcagtaagtcctgtcatctgccgtgggcgtcgggactggagactcttgactcgctgggtggagtgttagaggcctctggctattctactgaagtcgttgcgctgtcacgcctccaggggagcctgcaggacatgctgtggcagctggacctgtcacctggctgc

SEQ ID NO: 77 - DNA, 82 bp, Artificial Sequence
  Description of Artificial Sequence Synthetic oligonucleotide
cacagccagggcacattcactagcgattatagtaaatatctggattccaaggcagcgcacgattttgtagagtggctcttgc

SEQ ID NO: 78 - DNA, 117 bp, Artificial Sequence
  Description of Artificial Sequence Synthetic polynucleotide
cacagccagggcacattcactagcgattatagtaaatatctggattccaaggcagcgcacgattttgtagagtggctcttgaacggaggcccttcctccggagctccacctccgtcc

SEQ ID NO: 79 - DNA, 483 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgttctctgtctcaggaagacgctccgcagaccccgcgtccggttgctgaaatcgttccgtctttcatcaacaaagacaccgaaaccatcaacatgatgtctgaattcgttgctaacctgccgcaggaactgaaactgaccctgtctgaaatgcagccggctctgccgcagctgcagcagcacgttccggttctgaaagactcttctctgctgttcgaagaattcaaaaaactgatccgtaaccgtcagtctgaagctgctgactcttctccgtctgaactgaaatacctgggtctggacacccactctcgtaaaaaacgtcagctgtactctgctctggctaacaaatgctgccacgttggttgcaccaaacgttctctggctcgtttctgc

SEQ ID NO: 80 - DNA, 555 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagcggcacttctgagtctgctactccagaaagcggcccaggttctgaaccagcaacttctggctctgagactccaggcacttctgagtccgcaacgcctgaatccggtcctggttctgaaccagctacttccggcagcgaaaccccaggtaccggaggtggcgggagccaccatcaccaccaccacggaggtggcgggagctctgagtctgcgactccagagtctggtcctggtacttccactgagcctagcgagggttccgcaccaggttctccggctggtagcccgaccagcacggaggagggtacgtctgaatctgcaacgccggaatcgggcccaggttcggagggaggaggtggcgggagccgtaaaaaacgtcagctgtactctgctctggctaacaaatgctgccacgttggttgcaccaaacgttctctggctcgtttctgc

SEQ ID NO: 81 - DNA, 360 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgc

SEQ ID NO: 82 - DNA, 264 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgc

SEQ ID NO: 83 - DNA, 264 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctcgtcgtgaagctgaagacctgcaggttggtcaggttgaactgggtggtggtccgggtgctggttctctgcagccgctggctctggaaggttctctgcagaaacgtcagctgtactctgctctggctaacaaatgctgccacgttggttgcaccaaacgttctctggctcgtttctgc

SEQ ID NO: 84 - DNA, 222 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctcagctgtactctgctctggctaacaaatgctgccacgttggttgcaccaaacgttctctggctcgtttctgc

SEQ ID NO: 85 - DNA, 171 bp, Artificial Sequence
  Description of Artificial Sequence Synthetic polynucleotide
tttgtgaaccaacacctgtgcggctcagacctggtggaagctctctacctagtgtgcggggaacgaggcttcttctacacagaccccaccggcggagggccccgccggggcattgtggaacaatgctgtcacagcatctgctccctctaccagctggagaactactgcaac

SEQ ID NO: 86 - DNA, 111 bp, Artificial Sequence
  Description of Artificial Sequence Synthetic polynucleotide
catagccagggaaccttcacctccgactacagcaaataccttgacagtaggagagctcaggattttgtgcaatggctgatgaacacaaagaggaataaaaacaatatagcc

SEQ ID NO: 87 - DNA, 99 bp, Artificial Sequence
  Description of Artificial Sequence Synthetic oligonucleotide
cacggcgacggttcattctctgacgaaatgaatacaatactcgacaacctcgccgccagggactttatcaattggctcattcaaactaaaatcaccgac

SEQ ID NO: 88 - DNA, 102 bp, Artificial Sequence
  Description of Artificial Sequence Synthetic polynucleotide
atcaacgtgaagtgcagcctgccccagcagtgcatcaagccctgcaaggacgccggcatgcggttcggcaagtgcatgaacaagaagtgcaggtgctacagc

SEQ ID NO: 89 - DNA, 138 bp, Artificial Sequence
  Description of Artificial Sequence Synthetic polynucleotide
gctgacaacaaatgcgaaaactctctgcgtcgtgaaatcgcttgcggtcagtgccgtgacaaagttaaaaccgacggttacttctacgaatgctgcacctctgactctaccttcaaaaaatgccaggacctgctgcac

SEQ ID NO: 90 - DNA, 99 bp, Artificial Sequence
  Description of Artificial Sequence Synthetic oligonucleotide
gaatgcatcggtatgttcaaatcttgcgacccggaaaacgacaaatgctgcaaaggtcgtacctgctctcgtaaacaccgttggtgcaaatacaaactg

SEQ ID NO: 91 - DNA, 124 bp, Artificial Sequence
  Description of Artificial Sequence Synthetic polynucleotide
gaagttttgctatcataacactggcatgccttttcgaaatctcaagctcatcctacagggatgttcttcttcgtgcagtgaaacagaaaacaataagtgttgctcaacagacagatgcaacaaa

SEQ ID NO: 92 - DNA, 93 bp, Artificial Sequence
  Description of Artificial Sequence Synthetic oligonucleotide
gactcttggatggaagaagttatcaaactgtgcggtcgtgaactggttcgtgctcagatcgctatctgcggtatgtctacctggtctaaacgt

SEQ ID NO: 93 - DNA, 84 bp, Artificial Sequence
  Description of Artificial Sequence Synthetic oligonucleotide
cgtaaaaaacgtcagctgtactctgctctggctaacaaatgctgccacgttggttgcaccaaacgttctctggctcgtttctgc

SEQ ID NO: 94 - DNA, 81 bp, Artificial Sequence
  Description of Artificial Sequence Synthetic oligonucleotide
cattcacagggcacattcaccagtgactacagcaagtatctggactccaggcgtgcccaagattttgtgcagtggttgatg

SEQ ID NO: 95 - protein, 39 aa, Artificial Sequence
  Description of Artificial Sequence Synthetic polypeptide
HGEGTFTSDLSKQMEEEAVRLFIEWLKNGGPSSGAPPPS

SEQ ID NO: 96 - protein, 146 aa, Artificial Sequence
  Description of Artificial Sequence Synthetic polypeptide
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 97 - protein, 29 aa, Artificial Sequence
  Description of Artificial Sequence Synthetic peptide
HSQGTFTSDYSKYLDSKAAHDFVEWLLRA

SEQ ID NO: 98 - protein, 39 aa, Artificial Sequence
  Description of Artificial Sequence Synthetic polypeptide
HSQGTFTSDYSKYLDSKAAHDFVEWLLNGGPSSGAPPPS

SEQ ID NO: 99 - protein, 161 aa, Artificial Sequence
  Description of Artificial Sequence Synthetic polypeptide
DSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

SEQ ID NO: 100 - protein, 185 aa, Artificial Sequence
  Description of Artificial Sequence Synthetic polypeptide
DSWMEEVIKLCGRELVRAQIAICGMSTWSKRGGGGSGTSESATPESGPGSEPATSGSETPGTSESATPESGPGSEPATSGSETPGTGGGGSHHHHHHGGGGSSESATPESGPGTSTEPSEGSAPGSPAGSPTSTEEGTSESATPESGPGSEGGGGGSRKKRQLYSALANKCCHVGCTKRSLARFC

SEQ ID NO: 101 - protein, 120 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFC

SEQ ID NO: 102 - protein, 88 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFC

SEQ ID NO: 103 - protein, 88 aa, Artificial Sequence
  Description of Artificial Sequence Synthetic polypeptide
DSWMEEVIKLCGRELVRAQIAICGMSTWSRREAEDLQVGQVELGGGPGAGSLQPLALEGSLQKRQLYSALANKCCHVGCTKRSLARFC

SEQ ID NO: 104 - protein, 74 aa, Artificial Sequence
  Description of Artificial Sequence Synthetic polypeptide
DSWMEEVIKLCGRELVRAQIAICGMSTWSSGSETPGTSESATPESGPGSPQLYSALANKCCHVGCTKRSLARFC

SEQ ID NO: 105 - protein, 57 aa, Artificial Sequence
  Description of Artificial Sequence Synthetic polypeptide
FVNQHLCGSDLVEALYLVCGERGFFYTDPTGGGPRRGIVEQCCHSICSLYQLENYCN

SEQ ID NO: 106 - protein, 37 aa, Artificial Sequence
  Description of Artificial Sequence Synthetic polypeptide
HSQGTFTSDYSKYLDSRRAQDFVQWLMNTKRNKNNIA

SEQ ID NO: 107 - protein, 33 aa, Artificial Sequence
  Description of Artificial Sequence Synthetic polypeptide
HGDGSFSDEMNTILDNLAARDFINWLIQTKITD

SEQ ID NO: 108 - protein, 34 aa, Artificial Sequence
  Description of Artificial Sequence Synthetic polypeptide
INVKCSLPQQCIKPCKDAGMRFGKCMNKKCRCYS

SEQ ID NO: 109 - protein, 46 aa, Artificial Sequence
  Description of Artificial Sequence Synthetic polypeptide
ADNKCENSLRREIACGQCRDKVKTDGYFYECCTSDSTFKKCQDLLH

SEQ ID NO: 110 - protein, 33 aa, Artificial Sequence
  Description of Artificial Sequence Synthetic polypeptide
ECIGMFKSCDPENDKCCKGRTCSRKHRWCKYKL

SEQ ID NO: 111 - protein, 57 aa, Artificial Sequence
  Description of Artificial Sequence Synthetic polypeptide
LKCYQHGKVVTCHRDMKFCYHNTGMPFRNLKLILQGCSSSCSETENNKCCSTDRCNK

SEQ ID NO: 112 - protein, 31 aa, Artificial Sequence
  Description of Artificial Sequence Synthetic polypeptide
DSWMEEVIKLCGRELVRAQIAICGMSTWSKR

SEQ ID NO: 113 - protein, 28 aa, Artificial Sequence
  Description of Artificial Sequence Synthetic peptide
RKKRQLYSALANKCCHVGCTKRSLARFC

SEQ ID NO: 114 - protein, 27 aa, Artificial Sequence
  Description of Artificial Sequence Synthetic peptide
HSQGTFTSDYSKYLDSRRAQDFVQWLM

SEQ ID NO: 115 - protein, 50 aa, Artificial Sequence
  Description of Artificial Sequence Synthetic polypeptide
  MISC_FEATURE (1)..(50): This sequence may encompass 1-10 ′Gly Gly Gly Gly Ser′ repeating units
GGGGSGGGGSGGGGSGGGGSGGGGSGGGGSGGGGSGGGGSGGGGSGGGGS

SEQ ID NO: 116 - protein, 50 aa, Artificial Sequence
  Description of Artificial Sequence Synthetic polypeptide
  MISC_FEATURE (1)..(50): This sequence may encompass 1-10 ′Gly Gly Gly Gly Gly′ repeating units
GGGGGGGGGGGGGGGGGGGGGGGGGGGGGGGGGGGGGGGGGGGGGGGGGG

SEQ ID NO: 117 - protein, 17 aa, Artificial Sequence
  Description of Artificial Sequence Synthetic peptide
NGGPSSGAPPPSGGGGG

SEQ ID NO: 118 - protein, 16 aa, Artificial Sequence
  Description of Artificial Sequence Synthetic peptide
GGPSSGAPPPSGGGGG

SEQ ID NO: 119 - protein, 18 aa, Artificial Sequence
  Description of Artificial Sequence Synthetic peptide
GGSGAKLAALKAKLAALK

SEQ ID NO: 120 - protein, 18 aa, Artificial Sequence
  Description of Artificial Sequence Synthetic peptide
ELAALEAELAALEAGGSG

SEQ ID NO: 121 - protein, 5 aa, Artificial Sequence
  Description of Artificial Sequence Synthetic peptide
GGGGG

SEQ ID NO: 122 - protein, 5 aa, Artificial Sequence
  Description of Artificial Sequence Synthetic peptide
GGGGS

SEQ ID NO: 123 - protein, 116 aa, Artificial Sequence
  Description of Artificial Sequence Synthetic polypeptide
GTSESATPESGPGSEPATSGSETPGTSESATPESGPGSEPATSGSETPGTGGGGSHHHHHHGGGGSSESATPESGPGTSTEPSEGSAPGSPAGSPTSTEEGTSESATPESGPGSEG

SEQ ID NO: 124 - protein, 51 aa, Artificial Sequence
  Description of Artificial Sequence Synthetic polypeptide
SGSETPGTSESATPESGGGGGSHHHHHHGGGGSPGSPGPGTSTEPSEGSAP

SEQ ID NO: 125 - protein, 35 aa, Artificial Sequence
  Description of Artificial Sequence Synthetic polypeptide
RREAEDLQVGQVELGGGPGAGSLQPLALEGSLQKR

SEQ ID NO: 126 - protein, 21 aa, Artificial Sequence
  Description of Artificial Sequence Synthetic peptide
SGSETPGTSESATPESGPGSP

SEQ ID NO: 127 - DNA, 117 bp, Artificial Sequence; identical to SEQ ID NO: 75
  Description of Artificial Sequence Synthetic polynucleotide

SEQ ID NO: 128 - DNA, 438 bp, Artificial Sequence; identical to SEQ ID NO: 76
  Description of Artificial Sequence Synthetic polynucleotide

SEQ ID NO: 129 - DNA, 82 bp, Artificial Sequence; identical to SEQ ID NO: 77
  Description of Artificial Sequence Synthetic oligonucleotide

SEQ ID NO: 130 - DNA, 117 bp, Artificial Sequence; identical to SEQ ID NO: 78
  Description of Artificial Sequence Synthetic polynucleotide

SEQ ID NO: 131 - DNA, 483 bp, Artificial Sequence; identical to SEQ ID NO: 79
  Description of Artificial Sequence Synthetic polynucleotide

SEQ ID NO: 132 - DNA, 555 bp, Artificial Sequence; identical to SEQ ID NO: 80
  Description of Artificial Sequence Synthetic polynucleotide

SEQ ID NO: 133 - DNA, 360 bp, Artificial Sequence; identical to SEQ ID NO: 81
  Description of Artificial Sequence Synthetic polynucleotide

SEQ ID NO: 134 - DNA, 264 bp, Artificial Sequence; identical to SEQ ID NO: 82
  Description of Artificial Sequence Synthetic polynucleotide

SEQ ID NO: 135 - DNA, 264 bp, Artificial Sequence; identical to SEQ ID NO: 83
  Description of Artificial Sequence Synthetic polynucleotide

SEQ ID NO: 136 - DNA, 222 bp, Artificial Sequence; identical to SEQ ID NO: 84
  Description of Artificial Sequence Synthetic polynucleotide

SEQ ID NO: 137 - DNA, 171 bp, Artificial Sequence; identical to SEQ ID NO: 85
  Description of Artificial Sequence Synthetic polynucleotide

SEQ ID NO: 138 - DNA, 111 bp, Artificial Sequence; identical to SEQ ID NO: 86
  Description of Artificial Sequence Synthetic polynucleotide

SEQ ID NO: 139 - DNA, 99 bp, Artificial Sequence; identical to SEQ ID NO: 87
  Description of Artificial Sequence Synthetic oligonucleotide

SEQ ID NO: 140 - DNA, 102 bp, Artificial Sequence; identical to SEQ ID NO: 88
  Description of Artificial Sequence Synthetic polynucleotide

SEQ ID NO: 141 - DNA, 138 bp, Artificial Sequence; identical to SEQ ID NO: 89
  Description of Artificial Sequence Synthetic polynucleotide

SEQ ID NO: 142 - DNA, 99 bp, Artificial Sequence; identical to SEQ ID NO: 90
  Description of Artificial Sequence Synthetic oligonucleotide

SEQ ID NO: 143 - DNA, 124 bp, Artificial Sequence; identical to SEQ ID NO: 91
  Description of Artificial Sequence Synthetic polynucleotide

SEQ ID NO: 144 - protein, 39 aa, Artificial Sequence; identical to SEQ ID NO: 95
  Description of Artificial Sequence Synthetic polypeptide

SEQ ID NO: 145 - protein, 146 aa, Artificial Sequence; identical to SEQ ID NO: 96
  Description of Artificial Sequence Synthetic polypeptide

SEQ ID NO: 146 - protein, 29 aa, Artificial Sequence
  Description of Artificial Sequence Synthetic peptide
HSQGTFTSDYSKYLDSKAAHDFVEWLLRA

SEQ ID NO: 147 - protein, 39 aa, Artificial Sequence; identical to SEQ ID NO: 98
  Description of Artificial Sequence Synthetic polypeptide

SEQ ID NO: 148 - protein, 161 aa, Artificial Sequence; identical to SEQ ID NO: 99
  Description of Artificial Sequence Synthetic polypeptide

SEQ ID NO: 149 - protein, 185 aa, Artificial Sequence; identical to SEQ ID NO: 100
  Description of Artificial Sequence Synthetic polypeptide

SEQ ID NO: 150 - protein, 120 aa, Artificial Sequence; identical to SEQ ID NO: 101
  Description of Artificial Sequence Synthetic polypeptide

SEQ ID NO: 151 - protein, 88 aa, Artificial Sequence; identical to SEQ ID NO: 102
  Description of Artificial Sequence Synthetic polypeptide

SEQ ID NO: 152 - protein, 88 aa, Artificial Sequence; identical to SEQ ID NO: 103
  Description of Artificial Sequence Synthetic polypeptide

SEQ ID NO: 153 - protein, 74 aa, Artificial Sequence; identical to SEQ ID NO: 104
  Description of Artificial Sequence Synthetic polypeptide

SEQ ID NO: 154 - protein, 57 aa, Artificial Sequence; identical to SEQ ID NO: 105
  Description of Artificial Sequence Synthetic polypeptide

SEQ ID NO: 155 - protein, 37 aa, Artificial Sequence; identical to SEQ ID NO: 106
  Description of Artificial Sequence Synthetic polypeptide

SEQ ID NO: 156 - protein, 33 aa, Artificial Sequence; identical to SEQ ID NO: 107
  Description of Artificial Sequence Synthetic polypeptide

SEQ ID NO: 157 - protein, 34 aa, Artificial Sequence; identical to SEQ ID NO: 108
  Description of Artificial Sequence Synthetic polypeptide

SEQ ID NO: 158 - protein, 46 aa, Artificial Sequence; identical to SEQ ID NO: 109
  Description of Artificial Sequence Synthetic polypeptide

SEQ ID NO: 159 - protein, 33 aa, Artificial Sequence; identical to SEQ ID NO: 110
  Description of Artificial Sequence Synthetic polypeptide

SEQ ID NO: 160 - protein, 57 aa, Artificial Sequence; identical to SEQ ID NO: 111
  Description of Artificial Sequence Synthetic polypeptide

SEQ ID NO: 161 - DNA, 1485 bp, Artificial Sequence
  Description of Artificial Sequence Synthetic polynucleotide
cacggagaaggaacatttaccagcgacctcagcaagcagatggaggaagaggccgtgaggctgttcatcgagtggctgaagaacggcggaccctcctctggcgctccaccccctagcgggggtggcggaagccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 162 - DNA, 795 bp, Artificial Sequence
  Description of Artificial Sequence Synthetic polynucleotide
cattcacagggcacattcaccagtgactacagcaagtatctggactccaggcgtgcccaagattttgtgcagtggttgatggatgaagggggtggcgaagcagctgctaaggaggcagccgcaaaggaagcagctgcaaaggcaggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttgataa

SEQ ID NO: 163 - DNA, 747 bp, Artificial Sequence
  Description of Artificial Sequence Synthetic polynucleotide
cattcacagggcacattcaccagtgactacagcaagtatctggactccaggcgtgcccaagattttgtgcagtggttgatggatgaagggggtggcggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttgataa

SEQ ID NO: 164 - DNA, 1503 bp, Artificial Sequence
  Description of Artificial Sequence Synthetic polynucleotide
cattcacagggcacattcaccagtgactacagcaagtatctggactccaggcgtgcccaagattttgtgcagtggttgatggatgaagggggtggcgaagcagctgctaaggaggcagccgcaaaggaagcagctgcaaaggcaggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 165 - DNA, 1488 bp, Artificial Sequence
  Description of Artificial Sequence Synthetic polynucleotide
cacggccagggcacattcactagcgattatagtaaatatctggattccaaggcagcgcacgattttgtagagtggctcttggggggtggcgaagcagctgctaaggaggcagccgcaaaggaagcagctgcaaaggcaggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 166 - DNA, 1473 bp, Artificial Sequence
  Description of Artificial Sequence Synthetic polynucleotide
cacggccagggcacattcactagcgattatagtaaatatctggattccaaggcagcgcacgattttgtagagtggctcttgggaggaggtggaaaggccgcagctgaaaaagcagccgctgaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 167 - DNA, 1512 bp, Artificial Sequence
  Description of Artificial Sequence Synthetic polynucleotide
cacggccagggcacattcactagcgattatagtaaatatctggattccaaggcagcgcacgattttgtagagtggctcttggggggtggctccgggtccgagacacccggaaccagcgagtccgccacaccagagagcggccccggctctccaggaggcgaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 168 - DNA, 789 bp, Artificial Sequence
  Description of Artificial Sequence Synthetic polynucleotide
cacggccagggcacattcactagcgattatagtaaatatctggattccaaggcagcgcacgattttgtagagtggctcttggggggtggcgaagcagctgctaaggaggcagccgcaaaggaagcagctgcaaaggcaggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttgataa

SEQ ID NO: 169 - DNA, 804 bp, Artificial Sequence
  Description of Artificial Sequence Synthetic polynucleotide
cacagccagggcacattcactagcgattatagtaaatatctggattccaaggcagcgcacgattttgtagagtggctcttggggggtggctccgggtccgagacacccggaaccagcgagtccgccacaccagagagcggccccggctctccaggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgttgataa

SEQ ID NO: 170 - DNA, 1665 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggaggcccttcctccggagctccacctccgtccgggggtggcggaagcccggggagcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 171 - DNA, 1767 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggcgggagccgtaaaaaacgtcagctgtactctgctctggctaacaaatgctgccacgttggttgcaccaaacgttctctggctcgtttctgcgggggtggcgaagcagctgctaaggaggcagccgcaaaggaagcagctgcaaaggcaggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 172 - DNA, 996 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcggaggcccttcctccggagctccacctccgtccgggggtggcggaagcccggggagcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacaca

SEQ ID NO: 173 - DNA, 1650 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggaggcagcgggggaggcgggtccggaggcgggggatctggcgggggaggcagtgggggaggcgggagcggaggcgggggctctcagctgtactctgctctggctaacaaatgctgccacgttggttgcaccaaacgttctctggctcgtttctgcggaggcccttcctccggagctccacctccgtccgggggtggcggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 174 - DNA, 1650 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggaggcagcgggggaggcgggtccggaggcgggggatctggcgggggaggcagtgggggaggcgggagcggaggcgggggccctgcgctgtactctgctctggctaacaaatgctgccacgttggttgcaccaaacgttctctggctcgtttctgcggaggcccttcctccggagctccacctccgtccgggggtggcggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 175 - DNA, 1587 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggtgggagcgggggaggcggacagctgtactctgctctggctaacaaatgctgccacgttggttgcaccaaacgttctctggctcgtttctgcggaggcccttcctccggagctccacctccgtccgggggtggcggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 176 - DNA, 1587 bp, Artificial Sequence
  Description of Artificial Sequence Synthetic polynucleotide
gattcatggatggaggaggtcatcaaactgtgtggcagggagctggtgagagcacagatcgctatctgtgggatgagcacctggagtggcgggggagggagcgggggaggcggacagctgtactctgcactggccaataaatgctgccacgtgggatgtaccaagagatctctggcacggttttgtggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 177 - DNA, 918 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggtgggagcgggggaggcggacagctgtactctgctctggctaacaaatgctgccacgttggttgcaccaaacgttctctggctcgtttctgcggaggcccttcctccggagctccacctccgtccgggggtggcggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacaca

SEQ ID NO: 178 - DNA, 918 bp, Artificial Sequence
  Description of Artificial Sequence Synthetic polynucleotide
gattcatggatggaggaggtcatcaaactgtgtggcagggagctggtgagagcacagatcgctatctgtgggatgagcacctggagtggcgggggagggagcgggggaggcggacagctgtactctgcactggccaataaatgctgccacgtgggatgtaccaagagatctctggcacggttttgtggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacaca

SEQ ID NO: 179 - DNA, 1614 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggaggcggatccgggggcgggggttccggcgggggtgggagcgggggaggccagctgtactctgctctggctaacaaatgctgccacgttggttgcaccaaacgttctctggctcgtttctgcggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 180 - DNA, 1536 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggagggccccgccggcagctgtactctgctctggctaacaaatgctgccacgttggttgcaccaaacgttctctggctcgtttctgcgggggtggcggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 181 - DNA, 1569 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggagggccccgccggcagctgtactctgctctggctaacaaatgctgccacgttggttgcaccaaacgttctctggctcgtttctgcggaggcccttcctccggagctccacctccgtccgggggtggcggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 182 - DNA, 1584 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggagggccccgccggcagctgtactctgctctggctaacaaatgctgccacgttggttgcaccaaacgttctctggctcgtttctgcgggggtggcgaagcagctgctaaggaggcagccgcaaaggaagcagctgcaaaggcaggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 183 - DNA, 864 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggagggccccgccggcagctgtactctgctctggctaacaaatgctgccacgttggttgcaccaaacgttctctggctcgtttctgcggaggcccttcctccggagctccacctccgtccggcggaggtgggggtgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 184 - DNA, 879 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggagggccccgccggcagctgtactctgctctggctaacaaatgctgccacgttggttgcaccaaacgttctctggctcgtttctgcgggggtggcgaagcagctgctaaggaggcagccgcaaaggaagcagctgcaaaggcaggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 185 - DNA, 1485 bp, Artificial Sequence
  Description of Artificial Sequence Synthetic polynucleotide
catggtgaagggacctttaccagtgatgtaagttcttatttggaaggccaagctgccaaggaattcattgcttggctggtgaaaggcggaccctcctctggcgctccaccccctagcgggggtggcggaagccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 186 - DNA, 1488 bp, Artificial Sequence
  Description of Artificial Sequence Synthetic polynucleotide
catggtgaagggacctttaccagtgatgtaagttcttatttggaaggccaagctgccaaggaattcattgcttggctggtgaaaggcggcggaggcggatccggaggcggaggttccggcgggggtgggagcgggcaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 187 - DNA, 1500 bp, Artificial Sequence
  Description of Artificial Sequence Synthetic polynucleotide
cacggcgacggttcattctctgacgaaatgaatacaatactcgacaacctcgccgccagggactttatcaattggctcattcaaactaaaatcaccgacggaggcccttcctccggagctccacctccgtccgggggtggcggaagccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 188 - DNA, 1467 bp, Artificial Sequence
  Description of Artificial Sequence Synthetic polynucleotide
cacggcgacggttcattctctgacgaaatgaatacaatactcgacaacctcgccgccagggactttatcaattggctcattcaaactaaaatcaccgacgggggtggcggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 189 - DNA, 1515 bp, Artificial Sequence
  Description of Artificial Sequence Synthetic polynucleotide
cacggcgacggttcattctctgacgaaatgaatacaatactcgacaacctcgccgccagggactttatcaattggctcattcaaactaaaatcaccgacgggggtggcgaagcagctgctaaggaggcagccgcaaaggaagcagctgcaaaggcaggaggccaggtgaccctgcgcgagtccggccctgcactggtgaagcccacccagaccctgaccctgacctgcaccttctccggcttctccctgtccacctccggcatgtccgtgggctggatccggcagcctcccggcaaggccctggagtggctggcgactggctgaatggcaaggagtacaagtgcaaggtctccaacaaaggcctcccaagctccatcgagaaatgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccagaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 190 - DNA, 801 bp, Artificial Sequence
  Description of Artificial Sequence Synthetic polynucleotide
cacggcgacggttcattctctgacgaaatgaatacaatactcgacaacctcgccgccagggactttatcaattggctcattcaaactaaaatcaccgacgggggtggcgaagcagctgctaaggaggcagccgcaaaggaagcagctgcaaaggcaggaggcgacatccagatgacccagtccccctccaccctgtccgcctccgtgggcgaccgcgtgaccatcacctgcaagtgccagctgtccgtgggctacatgcactggtaccagcagaagcccggcaaggcccccaagctgctgatctacgacacctccaagctggcctccggcgtgccctcccgcttctccggctccggctccggcaccgagttcaccctgaccatctcctccctgcagcccgacgacttcgccacctactactgcttccagggctccggctaccccttcaccttcggcggcggcaccaagctggagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 191 - DNA, 1794 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctaaacgtggaggtggcgggagctctggcagcgaaaccccgggtacctccgaatctgctacaccggaaagcggtggaggtggcgggagccaccatcaccaccaccacggaggtggcgggagccctggcagccctggtccgggcactagcaccgagccatcggagggctccgcaccaggaggtggacatcatcaccatcatcatcaccatggaggtggcgggagccgtaaaaaacgtcagctgtactctgctctggctaacaaatgctgccacgttggttgcaccaaacgttctctggctcgtttctgcgggggtggcgaagcagctgctaaggaggcagccgcaaaggaagcagctgcaaaggcaggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 192 - protein, 493 aa, Artificial Sequence
  Description of Artificial Sequence Synthetic polypeptide
HGEGTFTSDLSKQMEEEAVRLFIEWLKNGGPSSGAPPPSGGGGSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 263 aa, Artificial Sequence
  Description of Artificial Sequence Synthetic polypeptide
HSQGTFTSDYSKYLDSRRAQDFVQWLMDEGGGEAAAKEAAAKEAAAKAGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 247 aa, Artificial Sequence
  Description of Artificial Sequence Synthetic polypeptide
HSQGTFTSDYSKYLDSRRAQDFVQWLMDEGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 499 aa, Artificial Sequence
  Description of Artificial Sequence Synthetic polypeptide
HSQGTFTSDYSKYLDSRRAQDFVQWLMDEGGGEAAAKEAAAKEAAAKAGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 494 aa, Artificial Sequence
  Description of Artificial Sequence Synthetic polypeptide
HGQGTFTSDYSKYLDSKAAHDFVEWLLGGGEAAAKEAAAKEAAAKAGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 489 aa, Artificial Sequence
  Description of Artificial Sequence Synthetic polypeptide
HGQGTFTSDYSKYLDSKAAHDFVEWLLGGGGKAAAEKAAAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 502 aa, Artificial Sequence
  Description of Artificial Sequence Synthetic polypeptide
HGQGTFTSDYSKYLDSKAAHDFVEWLLGGGSGSETPGTSESATPESGPGSPGGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 261 aa, Artificial Sequence
  Description of Artificial Sequence Synthetic polypeptide
HGQGTFTSDYSKYLDSKAAHDFVEWLLGGGEAAAKEAAAKEAAAKAGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 266 aa, Artificial Sequence
  Description of Artificial Sequence Synthetic polypeptide
HSQGTFTSDYSKYLDSKAAHDFVEWLLGGGSGSETPGTSESATPESGPGSPGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 553 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGPSSGAPPPSGGGGSPGSLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 587 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGGSRKKRQLYSALANKCCHVGCTKRSLARFCGGGEAAAKEAAAKEAAAKAGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 332 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGPSSGAPPPSGGGGSPGSLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 204 - protein, 548 aa, Artificial Sequence
  Description of Artificial Sequence Synthetic polypeptide
DSWMEEVIKLCGRELVRAQIAICGMSTWSGGGGSGGGGSGGGGSGGGGSGGGGSGGGGSQLYSALANKCCHVGCTKRSLARFCGGPSSGAPPPSGGGGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 548 aa, Artificial Sequence
  Description of Artificial Sequence Synthetic polypeptide
DSWMEEVIKLCGRELVRAQIAICGMSTWSGGGGSGGGGSGGGGSGGGGSGGGGSGGGGPALYSALANKCCHVGCTKRSLARFCGGPSSGAPPPSGGGGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 527 aa, Artificial Sequence
  Description of Artificial Sequence Synthetic polypeptide
DSWMEEVIKLCGRELVRAQIAICGMSTWSGGGGSGGGGQLYSALANKCCHVGCTKRSLARFCGGPSSGAPPPSGGGGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 527 aa, Artificial Sequence
  Description of Artificial Sequence Synthetic polypeptide
DSWMEEVIKLCGRELVRAQIAICGMSTWSGGGGSGGGGQLYSALANKCCHVGCTKRSLARFCGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 306 aa, Artificial Sequence
  Description of Artificial Sequence Synthetic polypeptide
DSWMEEVIKLCGRELVRAQIAICGMSTWSGGGGSGGGGQLYSALANKCCHVGCTKRSLARFCGGPSSGAPPPSGGGGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 209 - protein, 306 aa, Artificial Sequence
  Description of Artificial Sequence Synthetic polypeptide
DSWMEEVIKLCGRELVRAQIAICGMSTWSGGGGSGGGGQLYSALANKCCHVGCTKRSLARFCGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 210 - protein, 536 aa, Artificial Sequence
  Description of Artificial Sequence Synthetic polypeptide
DSWMEEVIKLCGRELVRAQIAICGMSTWSGGGGSGGGGSGGGGSGGGQLYSALANKCCHVGCTKRSLARFCGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 510 aa, Artificial Sequence
  Description of Artificial Sequence Synthetic polypeptide
DSWMEEVIKLCGRELVRAQIAICGMSTWSGGGPRRQLYSALANKCCHVGCTKRSLARFCGGGGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 521 aa, Artificial Sequence
  Description of Artificial Sequence Synthetic polypeptide
DSWMEEVIKLCGRELVRAQIAICGMSTWSGGGPRRQLYSALANKCCHVGCTKRSLARFCGGPSSGAPPPSGGGGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 526 aa, Artificial Sequence
  Description of Artificial Sequence Synthetic polypeptide
DSWMEEVIKLCGRELVRAQIAICGMSTWSGGGPRRQLYSALANKCCHVGCTKRSLARFCGGGEAAAKEAAAKEAAAKAGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 288 aa, Artificial Sequence
  Description of Artificial Sequence Synthetic polypeptide
DSWMEEVIKLCGRELVRAQIAICGMSTWSGGGPRRQLYSALANKCCHVGCTKRSLARFCGGPSSGAPPPSGGGGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 293 aa, Artificial Sequence
  Description of Artificial Sequence Synthetic polypeptide
DSWMEEVIKLCGRELVRAQIAICGMSTWSGGGPRRQLYSALANKCCHVGCTKRSLARFCGGGEAAAKEAAAKEAAAKAGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 493 aa, Artificial Sequence
  Description of Artificial Sequence Synthetic polypeptide
HGEGTFTSDVSSYLEGQAAKEFIAWLVKGGPSSGAPPPSGGGGSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 494 aa, Artificial Sequence
  Description of Artificial Sequence Synthetic polypeptide
HGEGTFTSDVSSYLEGQAAKEFIAWLVKGGGGGSGGGGSGGGGSGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 498 aa, Artificial Sequence
  Description of Artificial Sequence Synthetic polypeptide
HGDGSFSDEMNTILDNLAARDFINWLIQTKITDGGPSSGAPPPSGGGGSQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 487 aa, Artificial Sequence
  Description of Artificial Sequence Synthetic polypeptide
HGDGSFSDEMNTILDNLAARDFINWLIQTKITDGGGGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 503 aa, Artificial Sequence
  Description of Artificial Sequence Synthetic polypeptide
HGDGSFSDEMNTILDNLAARDFINWLIQTKITDGGGEAAAKEAAAKEAAAKAGGQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 267 aa, Artificial Sequence
  Description of Artificial Sequence Synthetic polypeptide
HGDGSFSDEMNTILDNLAARDFINWLIQTKITDGGGEAAAKEAAAKEAAAKAGGDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 598 aa, Artificial Sequence
  Description of Artificial Sequence Synthetic polypeptide
DSWMEEVIKLCGRELVRAQIAICGMSTWSKRGGGGSSGSETPGTSESATPESGGGGGSHHHHHHGGGGSPGSPGPGTSTEPSEGSAPGGGHHHHHHHHGGGGSRKKRQLYSALANKCCHVGCTKRSLARFCGGGEAAAKEAAAKEAAAKAGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - DNA, 249 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggaggcagcgggggaggcgggtccggaggcgggggatctggcgggggaggcagtgggggaggcgggagcggaggcgggggctctcagctgtactctgctctggctaacaaatgctgccacgttggttgcaccaaacgttctctggctcgtttctgc

SEQ ID NO: 224 - DNA, 249 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggaggcagcgggggaggcgggtccggaggcgggggatctggcgggggaggcagtgggggaggcgggagcggaggcgggggccctgcgctgtactctgctctggctaacaaatgctgccacgttggttgcaccaaacgttctctggctcgtttctgc

SEQ ID NO: 225 - DNA, 186 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcgggggtgggagcgggggaggcggacagctgtactctgctctggctaacaaatgctgccacgttggttgcaccaaacgttctctggctcgtttctgc

SEQ ID NO: 226 - DNA, 186 bp, Artificial Sequence
  Description of Artificial Sequence Synthetic polynucleotide
gattcatggatggaggaggtcatcaaactgtgtggcagggagctggtgagagcacagatcgctatctgtgggatgagcacctggagtggcgggggagggagcgggggaggcggacagctgtactctgcactggccaataaatgctgccacgtgggatgtaccaagagatctctggcacggttttgt

SEQ ID NO: 227 - DNA, 177 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggagggccccgccggcagctgtactctgctctggctaacaaatgctgccacgttggttgcaccaaacgttctctggctcgtttctgc

SEQ ID NO: 228 - DNA, 213 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtctggcggaggcggatccgggggcgggggttccggcgggggtgggagcgggggaggccagctgtactctgctctggctaacaaatgctgccacgttggttgcaccaaacgttctctggctcgtttctgc

SEQ ID NO: 229 - DNA, 84 bp, Artificial Sequence
  Description of Artificial Sequence Synthetic oligonucleotide
catggtgaagggacctttaccagtgatgtaagttcttatttggaaggccaagctgccaaggaattcattgcttggctggtgaaa

SEQ ID NO: 230 - protein, 83 aa, Artificial Sequence
  Description of Artificial Sequence Synthetic polypeptide
DSWMEEVIKLCGRELVRAQIAICGMSTWSGGGGSGGGGSGGGGSGGGGSGGGGSGGGGSQLYSALANKCCHVGCTKRSLARFC

SEQ ID NO: 231 - protein, 83 aa, Artificial Sequence
  Description of Artificial Sequence Synthetic polypeptide
DSWMEEVIKLCGRELVRAQIAICGMSTWSGGGGSGGGGSGGGGSGGGGSGGGGSGGGGPALYSALANKCCHVGCTKRSLARFC

SEQ ID NO: 232 - protein, 62 aa, Artificial Sequence
  Description of Artificial Sequence Synthetic polypeptide
DSWMEEVIKLCGRELVRAQIAICGMSTWSGGGGSGGGGQLYSALANKCCHVGCTKRSLARFC

SEQ ID NO: 233 - protein, 62 aa, Artificial Sequence; identical to SEQ ID NO: 232
  Description of Artificial Sequence Synthetic polypeptide

SEQ ID NO: 234 - protein, 59 aa, Artificial Sequence
  Description of Artificial Sequence Synthetic polypeptide
DSWMEEVIKLCGRELVRAQIAICGMSTWSGGGPRRQLYSALANKCCHVGCTKRSLARFC

SEQ ID NO: 235 - protein, 70 aa, Artificial Sequence
  Description of Artificial Sequence Synthetic polypeptide
DSWMEEVIKLCGRELVRAQIAICGMSTWSGGGGSGGGGSGGGGSGGGQLYSALANKCCHVGCTKRSLARF

SEQ ID NO: 236 - protein, 28 aa, Artificial Sequence
  Description of Artificial Sequence Synthetic peptide
HGEGTFTSDVSSYLEGQAAKEFIAWLVK

SEQ ID NO: 237 - protein, 15 aa, Artificial Sequence
  Description of Artificial Sequence Synthetic peptide
EAAAKEAAAKEAAAK

SEQ ID NO: 238 - protein, 16 aa, Artificial Sequence
  Description of Artificial Sequence Synthetic peptide
GGPSSGAPPPSGGGGS

SEQ ID NO: 239 - protein, 21 aa, Artificial Sequence
  Description of Artificial Sequence Synthetic peptide
SGSETPGTSESATPESGPGSP

SEQ ID NO: 240 - protein, 35 aa, Artificial Sequence
  Description of Artificial Sequence Synthetic polypeptide
SGSETPGTSESATPESGPGSPGPGTSTEPSEGSAP

SEQ ID NO: 241 - protein, 30 aa, Artificial Sequence
  Description of Artificial Sequence Synthetic polypeptide
GGGGSGGGGSGGGGSGGGGSGGGGSGGGGS

SEQ ID NO: 242 - protein, 9 aa, Artificial Sequence
  Description of Artificial Sequence Synthetic peptide
GGGGSGGGG

SEQ ID NO: 243 - protein, 18 aa, Artificial Sequence
  Description of Artificial Sequence Synthetic peptide
GGGGSGGGGSGGGGSGGG

SEQ ID NO: 244 - protein, 6 aa, Artificial Sequence
  Description of Artificial Sequence Synthetic peptide
GGGPRR

SEQ ID NO: 245 - DNA, 93 bp, Artificial Sequence; identical to SEQ ID NO: 92
  Description of Artificial Sequence Synthetic oligonucleotide

SEQ ID NO: 246 - DNA, 84 bp, Artificial Sequence; identical to SEQ ID NO: 93
  Description of Artificial Sequence Synthetic oligonucleotide

SEQ ID NO: 247 - DNA, 81 bp, Artificial Sequence; identical to SEQ ID NO: 94
  Description of Artificial Sequence Synthetic oligonucleotide

SEQ ID NO: 248 - DNA, 249 bp, Artificial Sequence; identical to SEQ ID NO: 223
  Description of Artificial Sequence Synthetic polynucleotide

SEQ ID NO: 249 - DNA, 249 bp, Artificial Sequence; identical to SEQ ID NO: 224
  Description of Artificial Sequence Synthetic polynucleotide

SEQ ID NO: 250 - DNA, 186 bp, Artificial Sequence; identical to SEQ ID NO: 225
  Description of Artificial Sequence Synthetic polynucleotide

SEQ ID NO: 251 - DNA, 186 bp, Artificial Sequence; identical to SEQ ID NO: 226
  Description of Artificial Sequence Synthetic polynucleotide

SEQ ID NO: 252 - DNA, 177 bp, Artificial Sequence; identical to SEQ ID NO: 227
  Description of Artificial Sequence Synthetic polynucleotide

SEQ ID NO: 253 - DNA, 213 bp, Artificial Sequence; identical to SEQ ID NO: 228
  Description of Artificial Sequence Synthetic polynucleotide

SEQ ID NO: 254 - DNA, 84 bp, Artificial Sequence; identical to SEQ ID NO: 229
  Description of Artificial Sequence Synthetic oligonucleotide

SEQ ID NO: 255 - protein, 31 aa, Artificial Sequence; identical to SEQ ID NO: 112
  Description of Artificial Sequence Synthetic polypeptide

SEQ ID NO: 256 - protein, 28 aa, Artificial Sequence
  Description of Artificial Sequence Synthetic peptide
RKKRQLYSALANKCCHVGCTKRSLARFC

SEQ ID NO: 257 - protein, 27 aa, Artificial Sequence
  Description of Artificial Sequence Synthetic peptide
HSQGTFTSDYSKYLDSRRAQDFVQWLM

SEQ ID NO: 258 - protein, 83 aa, Artificial Sequence; identical to SEQ ID NO: 230
  Description of Artificial Sequence Synthetic polypeptide

SEQ ID NO: 259 - protein, 83 aa, Artificial Sequence; identical to SEQ ID NO: 231
  Description of Artificial Sequence Synthetic polypeptide

SEQ ID NO: 260 - protein, 62 aa, Artificial Sequence; identical to SEQ ID NO: 232
  Description of Artificial Sequence Synthetic polypeptide

SEQ ID NO: 261 - protein, 62 aa, Artificial Sequence; identical to SEQ ID NO: 232
  Description of Artificial Sequence Synthetic polypeptide

SEQ ID NO: 262 - protein, 59 aa, Artificial Sequence; identical to SEQ ID NO: 234
  Description of Artificial Sequence Synthetic polypeptide

SEQ ID NO: 263 - protein, 70 aa, Artificial Sequence; identical to SEQ ID NO: 235
  Description of Artificial Sequence Synthetic polypeptide

SEQ ID NO: 264 - protein, 28 aa, Artificial Sequence
  Description of Artificial Sequence Synthetic peptide
HGEGTFTSDVSSYLEGQAAKEFIAWLVK

SEQ ID NO: 265 - DNA, 1419 bp, Artificial Sequence
  Description of Artificial Sequence Synthetic polynucleotide
gactcttggatggaagaagttatcaaactgtgcggtcgtgaactggttcgtgctcagatcgctatctgcggtatgtctacctggtcttctggcagcgaaaccccgggtacctccgaatctgctacaccggaaagcggtcctggcagccctggtccgggcactagcaccgagccatcggagggctccgcaccacagctgtactctgctctggctaacaaatgctgccacgttggttgcaccaaacgttctctggctcgtttctgcgggggtggcgaagcagctgctaaggaggcagccgcaaaggaagcagctgcaaaggcaggaggcctgcgcgagtccggccctgcactggtgaagcccacccagaccctgaccctgacctgcaccttctccggcttctccctgtccacctccggcatgtccgtgggctggatccggcagcctcccggcaaggccctggagtggctggctgacatctggtgggacgacaagaaggactacaacccctccctgaagtcccgcctgaccatctccaaggacacctccaagaaccaggtggtgctgaaggtgaccaacatggaccccgccgacaccgccacctactactgcgcccgctcaatgattaccaactggtacttcgacgtgtggggagccggtaccaccgtgaccgtgtcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctg

SEQ ID NO: 266 - protein, 473 aa, Artificial Sequence
  Description of Artificial Sequence Synthetic polypeptide
DSWMEEVIKLCGRELVRAQIAICGMSTWSSGSETPGTSESATPESGPGSPGPGTSTEPSEGSAPQLYSALANKCCHVGCTKRSLARFCGGGEAAAKEAAAKEAAAKAGGLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSL

SEQ ID NO: 267 - protein, 4 aa, Artificial Sequence
  Description of Artificial Sequence Synthetic peptide
RKKR

SEQ ID NO: 268 - protein, 29 aa, Artificial Sequence
  Description of Artificial Sequence Synthetic peptide
  MOD_RES (2)..(2): D-Ser
HSQGTFTSDYSKYLDECAAKEFICWLLRA

(19) United States
(12) Patent Application Publication
(10) Pub. No.: US 2017/0327577 A1
(43) Pub. Date: Nov. 16, 2017

(54) METHODS OF CONSTRUCTING AMINO TERMINAL IMMUNOGLOBULIN FUSION PROTEINS AND COMPOSITIONS THEREOF

(71) Applicant: The California Institute for Biomedical Research, La Jolla, CA (US)

(72) Inventors: Feng WANG, Carlsbad, CA (US); Yan LIU, San Diego, CA (US); Ying WANG, San Diego, CA (US); Guangsen FU, San Diego, CA (US); Peter G. SCHULTZ, La Jolla, CA (US)

(21) Appl. No.: 15/315,645

(22) PCT Filed: Jun. 5, 2015
(86) PCT No.: PCT/US15/34533

§ 371 Date: Dec. 1, 2016

Related U.S. Application Data
(60) Provisional application No. 62/009,054, filed on Jun. 6, 2014.
(60) Provisional application No. 62/030,526, filed on Jul. 29, 2014.
(60) Provisional application No. 62/064,186, filed on Oct. 15, 2014.

(51) Int. Cl. C07K 16/28; C07K 16/18; C07K 16/46; A61K 39/00; A61K 38/00

(52) U.S. Cl. CPC C07K 16/2803; C07K 16/18; C07K 16/2869; C07K 16/46; A61K 38/00; A61K 39/00; A61K 2039/505; C07K 2319/30

(57) ABSTRACT

Disclosed herein are immunoglobulin fusion proteins comprising a first immunoglobulin region attached to a therapeutic peptide at the amino terminus of the immunoglobulin region. The immunoglobulin fusion proteins may further comprise a second immunoglobulin region. The immunoglobulin fusion protein may further comprise one or more connecting peptides, linkers, proteolytic cleavage sites, internal linkers, or a combination thereof. The immunoglobulin fusion proteins may further comprise one or more additional therapeutic peptides. Also disclosed herein are compositions comprising the immunoglobulin fusion proteins and methods for using the immunoglobulin fusion proteins for the treatment or prevention of a disease or condition in a subject.

### CROSS-REFERENCE

This application is a U.S. National Stage entry of International Application No. PCT/US15/34533, filed Jun. 5, 2015, which claims the benefit of U.S. Provisional Application No. 62/009,054 filed Jun. 6, 2014; U.S. Provisional Application No. 62/030,526 filed Jul. 29, 2014; and U.S. Provisional Application No. 62/064,186 filed Oct. 15, 2014, which are all incorporated by reference in their entirety.

### SEQUENCE LISTING

The instant application contains a Sequence Listing which has been submitted electronically in ASCII format and is hereby incorporated by reference in its entirety. Said ASCII copy, created on Mar. 15, 2017, is named 41135-721_831_SL.txt and is 422,556 bytes in size.

### BACKGROUND OF THE INVENTION

Antibodies are natural proteins that the vertebrate immune system forms in response to foreign substances (antigens), primarily for defense against infection. For over a century, antibodies have been induced in animals under artificial conditions and harvested for use in therapy or diagnosis of disease conditions, or for biological research. Each individual immunoglobulin producing cell produces a single type of immunoglobulin with a chemically defined composition, however, antibodies obtained directly from animal serum in response to antigen inoculation actually comprise an ensemble of non-identical molecules (e.g., polyclonal antibodies) made from an ensemble of individual immunoglobulin producing cells.

### SUMMARY OF THE INVENTION

Disclosed herein are methods for producing immunoglobulin fusion proteins and compositions thereof. These methods and compositions find use in a number of applications, for example, for the treatment of various diseases and conditions. The methods and compositions may also be used to improve the delivery of a therapeutic peptide to target cells, tissues, or tumors.

Provided herein is an immunoglobulin fusion protein comprising: a first immunoglobulin region; a first therapeutic peptide not derived from an immunoglobulin; and a connecting peptide; wherein the connecting peptide connects the first therapeutic peptide to the amino terminus of the first immunoglobulin region. In one embodiment, the first immunoglobulin region comprises a variable region of an immunoglobulin light chain. In one embodiment, the first immunoglobulin region further comprises a constant region of an immunoglobulin light chain.

In one embodiment, the immunoglobulin fusion protein further comprises a second immunoglobulin region. In one embodiment, the second immunoglobulin region comprises a variable region of an immunoglobulin heavy chain. In one embodiment, the second immunoglobulin region further comprises a constant region of an immunoglobulin heavy chain.

In one embodiment, the first immunoglobulin region comprises a variable region of an immunoglobulin heavy chain. In one embodiment, the first immunoglobulin region further comprises a constant region of an immunoglobulin heavy chain. In one embodiment, the second immunoglobulin region comprises a variable region of an immunoglobulin light chain. In one embodiment, the second immunoglobulin region further comprises a constant region of an immunoglobulin light chain.

In one embodiment, the first immunoglobulin region comprises an amino acid sequence that is based on or derived from any one of SEQ ID NOs: 5-8. In one embodiment, the first immunoglobulin region comprises an amino acid sequence that is at least about or about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID NOs: 5-8. In one embodiment, the second immunoglobulin region comprises an amino acid sequence that is based on or derived from any one of SEQ ID NOs: 5-8. In one embodiment, the second immunoglobulin region comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID NOs: 5-8. In one embodiment, the first immunoglobulin region comprises an amino acid sequence that is based on or derived from a trastuzumab immunoglobulin. In one embodiment, the second immunoglobulin region comprises an amino acid sequence that is based on or derived from a trastuzumab immunoglobulin. In one embodiment, the first immunoglobulin region comprises an amino acid sequence that is based on or derived from a palivizumab immunoglobulin. In one embodiment, the second immunoglobulin region comprises an amino acid sequence that is based on or derived from a palivizumab immunoglobulin.

In one embodiment, the connecting peptide comprises from about 0 to about 50 amino acids. In one embodiment, the connecting peptide comprises from about 1 to about 50 amino acids. In one embodiment, the connecting peptide comprises from about 1 to about 20 amino acids, or about 0, 1, 2, 3, 4, 5, 6, 7, 8, 9, 10, 11, 12, 13, 14, 15, 16, 17, 18, 19 or 20 amino acids. In one embodiment, the amino acids of the connecting peptide do not form a regular secondary structure. In one embodiment, the connecting peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of any one of SEQ ID NOs: 115-118, 237-239. In one embodiment, the connecting peptide comprises an amino acid sequence that is at least about 80% identical to an amino acid sequence of any one of SEQ ID NOs: 115-118, 237-239.

In one embodiment, the activity of the therapeutic peptide in the immunoglobulin fusion protein is comparable to the activity of the therapeutic peptide in standard use formulations. In one embodiment, the activity of the first immunoglobulin region in the immunoglobulin fusion protein is comparable to the activity of the native first immunoglobulin region. In various embodiments, the activity of the therapeutic peptide in the immunoglobulin fusion protein is comparable to the activity of the therapeutic peptide in standard use formulations and the activity of the first immunoglobulin region in the immunoglobulin fusion protein is comparable to the activity of the native first immunoglobulin region.

Further provided herein are immunoglobulin fusion proteins comprising: a first immunoglobulin region; a first therapeutic peptide not derived from an immunoglobulin; and a connecting peptide; wherein the connecting peptide connects the first therapeutic peptide to the amino terminus of the first immunoglobulin region. In one embodiment, the activity of the therapeutic peptide in the immunoglobulin fusion protein is comparable to the activity of the therapeutic peptide in standard use formulations. In one embodiment, the activity of the first immunoglobulin region in the immunoglobulin fusion protein is comparable to the activity of the native first immunoglobulin region. In various embodiments, the activity of the therapeutic peptide in the immunoglobulin fusion protein is comparable to the activity of the therapeutic peptide in standard use formulations and the activity of the first immunoglobulin region in the immunoglobulin fusion protein is comparable to the activity of the native first immunoglobulin region. In one example, the activity of the immunoglobulin region of the immunoglobulin fusion protein is about or at least 10%, 20%, 30%, 40%, 50%, 60%, 70%, 80%, 90%, 100% of the activity of the immunoglobulin region of the immunoglobulin fusion protein without the therapeutic peptide and/or connecting peptide. In some embodiments, the immunoglobulin region of the immunoglobulin fusion protein has at least some activity for its cognate substrate (e.g., antigen). In some embodiments, the immunoglobulin region of the immunoglobulin fusion protein has little or no activity for its cognate substrate. In some embodiments, comparable activity indicates that the therapeutic peptide of the immunoglobulin fusion protein has an activity that the therapeutic peptide without the immunoglobulin region and/or connecting peptide has. In one example, the activity of the therapeutic peptide of the immunoglobulin fusion protein is about or at least 10%, 20%, 30%, 40%, 50%, 60%, 70%, 80%, 90%, 100% of the activity of the therapeutic peptide of the immunoglobulin fusion protein without the immunoglobulin region and/or connecting peptide. In some embodiments, the therapeutic peptide of the immunoglobulin fusion protein has enhanced activity for its cognate substrate (e.g., binding partner). In some embodiments, the therapeutic peptide has an activity that is about or at least about 110%, 120%, 140%, 160%, 180%, 200%, 250%, 300%, 400%, 450%, 500%, 550%, 600% or 800% of the activity of the therapeutic peptide without the immunoglobulin region and/or connecting peptide. In some embodiments, the amino acids of the connecting peptide do nor form a regular secondary structure, including alpha helices and beta strands.

Further provided herein are immunoglobulin fusion proteins comprising: a first immunoglobulin region; a first therapeutic peptide not derived from an immunoglobulin; and optionally a connecting peptide; wherein the optional connecting peptide connects the first therapeutic peptide to the amino terminus of the first immunoglobulin region. In one embodiment, the activity of the therapeutic peptide in the immunoglobulin fusion protein is comparable to the activity of the therapeutic peptide in standard use formulations. In one embodiment, the activity of the first immunoglobulin region in the immunoglobulin fusion protein is comparable to the activity of the native first immunoglobulin region. In various embodiments, the activity of the therapeutic peptide in the immunoglobulin fusion protein is comparable to the activity of the therapeutic peptide in standard use formulations and the activity of the first immunoglobulin region in the immunoglobulin fusion protein is comparable to the activity of the native first immunoglobulin region. In one example, the activity of the immunoglobulin region of the immunoglobulin fusion protein is about or at least 10%, 20%, 30%, 40%, 50%, 60%, 70%, 80%, 90%, 100% of the activity of the immunoglobulin region of the immunoglobulin fusion protein without the therapeutic peptide and/or optional connecting peptide. In some embodiments, the immunoglobulin region of the immunoglobulin fusion protein has at least some activity for its cognate substrate (e.g., antigen). In some embodiments, the immunoglobulin region of the immunoglobulin fusion protein has little or no activity for its cognate substrate. In some embodiments, comparable activity indicates that the therapeutic peptide of the immunoglobulin fusion protein has an activity that the therapeutic peptide without the immunoglobulin region and/or optional connecting peptide has. In one example, the activity of the therapeutic peptide of the immunoglobulin fusion protein is about or at least 10%, 20%, 30%, 40%, 50%, 60%, 70%, 80%, 90%, 100% of the activity of the therapeutic peptide of the immunoglobulin fusion protein without the immunoglobulin region and/or optional connecting peptide. In some embodiments, the therapeutic peptide of the immunoglobulin fusion protein has enhanced activity for its cognate substrate (e.g., binding partner). In some embodiments, the therapeutic peptide has an activity that is about or at least about 110%, 120%, 140%, 160%, 180%, 200%, 250%, 300%, 400%, 450%, 500%, 550%, 600% or 800% of the activity of the therapeutic peptide without the immunoglobulin region and/or optional connecting peptide. In some embodiments, the amino acids of the optional connecting peptide do nor form a regular secondary structure, including alpha helices and beta strands.

Further provided herein are immunoglobulin fusion proteins comprising: a first immunoglobulin region; and a first therapeutic peptide not derived from an immunoglobulin; wherein the first therapeutic peptide is connected to the amino terminus of the first immunoglobulin region. In one embodiment, the activity of the therapeutic peptide in the immunoglobulin fusion protein is comparable to the activity of the therapeutic peptide in standard use formulations. In one embodiment, the activity of the first immunoglobulin region in the immunoglobulin fusion protein is comparable to the activity of the native first immunoglobulin region. In various embodiments, the activity of the therapeutic peptide in the immunoglobulin fusion protein is comparable to the activity of the therapeutic peptide in standard use formulations and the activity of the first immunoglobulin region in the immunoglobulin fusion protein is comparable to the activity of the native first immunoglobulin region. In some embodiments, comparable activity indicates that the immunoglobulin region of the immunoglobulin fusion protein has an activity that the immunoglobulin region without the therapeutic peptide has. In one example, the activity of the immunoglobulin region of the immunoglobulin fusion protein is about or at least 10%, 20%, 30%, 40%, 50%, 60%, 70%, 80%, 90%, 100% of the activity of the immunoglobulin region of the immunoglobulin fusion protein without the therapeutic peptide. In some embodiments, the immunoglobulin region of the immunoglobulin fusion protein has at least some activity for its cognate substrate (e.g., antigen). In some embodiments, the immunoglobulin region of the immunoglobulin fusion protein has little or no activity for its cognate substrate. In one example, the activity of the therapeutic peptide of the immunoglobulin fusion protein is about or at least 10%, 20%, 30%, 40%, 50%, 60%, 70%, 80%, 90%, 100% of the activity of the therapeutic peptide of the immunoglobulin fusion protein without the immunoglobulin region. In some embodiments, the therapeutic peptide of the immunoglobulin fusion protein has enhanced activity for its cognate substrate (e.g., binding partner). In some embodiments, the therapeutic peptide has an activity that is about or at least about 110%, 120%, 140%, 160%, 180%, 200%, 250%, 300%, 400%, 450%, 500%, 550%, 600% or 800% of the activity of the therapeutic peptide without the immunoglobulin region.

In one aspect of the disclosure, the therapeutic peptide of the immunoglobulin fusion protein is a GLP-1 receptor agonist or a synthetic thereof. In one embodiment, the therapeutic peptide is configured to treat diabetes and/or a diabetes related disease. In one embodiment, the therapeutic peptide is configured to treat obesity and/or an obesity related disease. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of exendin-4, exenatide, or any synthetic thereof. In one embodiment, the therapeutic peptide comprises an amino acid sequence that is at least about 80% identical to an amino acid sequence of exendin-4, exenatide, or any synthetic thereof. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 95. In one embodiment, the therapeutic peptide comprises an amino acid sequence that is at least about 80% identical to an amino acid sequence of SEQ ID NO: 95. In one embodiment, the therapeutic peptide comprises from about 20 to about 100 amino acids comprising from about 20 to about 39 amino acids identical to SEQ ID NO: 95.

In one embodiment, the second immunoglobulin region has formula I: A²-E¹-T²-E², wherein A² is the second immunoglobulin region, E¹ is a first extender peptide, E² is a second extender peptide, and T² is a second therapeutic peptide. In one embodiment, E¹ comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 119. In one embodiment, E¹ comprises an amino acid sequence that is at least about 80% identical to an amino acid sequence of SEQ ID NO: 119. In one embodiment, wherein E¹ comprises from about 5 to about 50 amino acids comprising from about 5 to about 23 amino acids identical to an amino acid sequence of SEQ ID NO: 119. In one embodiment, E² comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 120. In one embodiment, E² comprises an amino acid sequence that is at least about 80% identical to an amino acid sequence of SEQ ID NO: 120. In one embodiment, E² comprises from about 5 to about 50 amino acids comprising from about 5 to about 23 amino acids identical to an amino acid sequence of SEQ ID NO: 120. In one embodiment, T² is a hormone. In one embodiment, T² is effective for the treatment of a metabolic disorder and/or a disease resulting from said metabolic disorder. In one embodiment, the metabolic disorder includes lipodystrophy, diabetes and hypertriglyceridemia. In one embodiment, T² comprises an amino acid sequence that is at least 50% identical to an amino acid sequence of leptin or an analog thereof including metreleptin. In one embodiment, T² comprises an amino acid sequence that is about or at least 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 96. In one embodiment, T² comprises an amino acid sequence that is at least about 80% identical to an amino acid sequence of SEQ ID NO: 96. In one embodiment, T² comprises from about 20 to about 200 amino acids comprising from about 5 to about 167 amino acids identical to an amino acid sequence of SEQ ID NO: 96.

In one embodiment, the second immunoglobulin region comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 43. In one embodiment, the second immunoglobulin region comprises an amino acid sequence that is at least about 80% identical to an amino acid sequence of SEQ ID NO: 43. In one embodiment, the second immunoglobulin region comprises an amino acid sequence that is at least 50% identical to an amino acid sequence of SEQ ID NO: 44. In one embodiment, the second immunoglobulin region comprises an amino acid sequence that is at least 80% identical to an amino acid sequence of SEQ ID NO: 44. Further provided herein is a method of treating an individual with obesity, comprising administering an immunoglobulin fusion protein. Further provided herein is a method of treating an individual with diabetes, comprising administering an immunoglobulin fusion protein.

In one aspect of the disclosure, the therapeutic peptide of the immunoglobulin fusion protein is a glucagon analog or a synthetic thereof. In one embodiment, the therapeutic peptide is configured to treat obesity or an obesity related disease. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 146. In one embodiment, the therapeutic peptide comprises an amino acid sequence that is at least about 80% identical to an amino acid sequence of SEQ ID NO: 146. In one embodiment, the therapeutic peptide comprises from about 5 to about 50 amino acids comprising from about 5 to about 29 amino acids identical to an amino acid sequence of SEQ ID NO: 146. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 147. In one embodiment, the therapeutic peptide comprises an amino acid sequence that is at least about 80% identical to an amino acid sequence of SEQ ID NO: 147. In one embodiment, the therapeutic peptide comprises from about 5 to about 50 amino acids comprising from about 5 to about 39 amino acids identical to an amino acid sequence of SEQ ID NO: 147. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 147. In one embodiment, the therapeutic peptide comprises an amino acid sequence that is at least about 80% identical to an amino acid sequence of SEQ ID NO: 147. In one embodiment, the therapeutic peptide comprises from about 5 to about 50 amino acids comprising from about 5 to about 39 amino acids identical to an amino acid sequence of SEQ ID NO: 147.

In one embodiment, the second immunoglobulin region has formula I: A²-E¹-T²-E², wherein A² is the second immunoglobulin region, E¹ is a first extender peptide, E² is a second extender peptide, and T² is a second therapeutic peptide. In one embodiment, E¹ comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 119. In one embodiment, E¹ comprises an amino acid sequence that is at least about 80% identical to an amino acid sequence of SEQ ID NO: 119. In one embodiment, E¹ comprises from about 5 to about 50 amino acids comprising from about 5 to about 23 amino acids identical to an amino acid sequence of SEQ ID NO: 119. In one embodiment, E² comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 120. In one embodiment, E² comprises an amino acid sequence that is at least about 80% identical to an amino acid sequence of SEQ ID NO: 120. In one embodiment, E² comprises from about 5 to about 50 amino acids comprising from about 5 to about 23 amino acids identical to an amino acid sequence of SEQ ID NO: 120. In one embodiment, T² is a hormone. In one embodiment, T² is effective for the treatment of a metabolic disorder and/or a disease resulting from said metabolic disorder. In one embodiment, the metabolic disorder includes lipodystrophy, diabetes and hypertriglyceridemia. In one embodiment, T² comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of leptin or an analog thereof including metreleptin. In one embodiment, T² comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 145. In one embodiment, T² comprises an amino acid sequence that is at least about 80% identical to an amino acid sequence of SEQ ID NO: 145. In one embodiment, T² comprises from about 20 to about 200 amino acids comprising from about 5 to about 167 amino acids identical to an amino acid sequence of SEQ ID NO: 145.

In one embodiment, the second immunoglobulin region comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of an amino acid sequence of SEQ ID NO: 44. In one embodiment, the second immunoglobulin region comprises an amino acid sequence that is at least about 80% identical to an amino acid sequence of SEQ ID NO: 44.

In one aspect of the disclosure, the therapeutic peptide of the immunoglobulin fusion protein is a hormone or a synthetic thereof. In one embodiment, therapeutic peptide is configured to treat diabetes and/or a diabetes related disease. In one embodiment, the therapeutic peptide is configured to treat obesity and/or an obesity related disease. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of insulin. In one embodiment, the therapeutic peptide comprises an amino acid sequence that is at least about 80% identical to an amino acid sequence of insulin. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 105. In one embodiment, the therapeutic peptide comprises an amino acid sequence that is at least about 80% identical to an amino acid sequence of SEQ ID NO: 105. In one embodiment, the therapeutic peptide comprises from about 20 to about 100 amino acids comprising from about 20 to about 57 amino acids identical to an amino acid sequence of SEQ ID NO: 105.

In one aspect of the disclosure, the therapeutic peptide of the immunoglobulin fusion protein comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of oxyntomodulin. In one embodiment, the therapeutic peptide comprises an amino acid sequence that is at least about 80% identical to an amino acid sequence of oxyntomodulin. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 106. In one embodiment, the therapeutic peptide comprises an amino acid sequence that is at least about 80% identical to an amino acid sequence of SEQ ID NO: 106. In one embodiment, the therapeutic peptide comprises from about 15 to about 100 amino acids comprising from about 15 to about 37 amino acids identical to an amino acid sequence of SEQ ID NO: 106.

In one aspect of the disclosure, the therapeutic peptide of the immunoglobulin fusion protein is configured to treat short bowel syndrome and/or a short bowel syndrome related disease. In one embodiment, the therapeutic peptide is configured to treat inflammatory bowel disease and/or an inflammatory bowel related disease. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of glucagon. In one embodiment, the therapeutic peptide comprises an amino acid sequence that is at least about 80% identical to an amino acid sequence of glucagon. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 107. In one embodiment, the therapeutic peptide comprises an amino acid sequence that is at least about 80% identical to an amino acid sequence of SEQ ID NO: 107. In one embodiment, the therapeutic peptide comprises from about 15 to about 200 amino acids comprising from about 15 to about 33 amino acids identical to an amino acid sequence of SEQ ID NO: 107. Further provided herein is a method of treating an individual with short bowel syndrome and/or a short bowel syndrome related disease, comprising administering an immunoglobulin fusion protein. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of a glucagon like protein (e.g., GLP2). In one embodiment, the therapeutic peptide comprises an amino acid sequence that is at least about 80% identical to an amino acid sequence of a glucagon like protein. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 156. Further provided herein is a method of treating an individual with an inflammatory bowel disease and/or an inflammatory bowel related disease, comprising administering an immunoglobulin fusion protein.

In one aspect of the disclosure, the therapeutic peptide of the immunoglobulin fusion protein binds to potassium channels. In one embodiment, the therapeutic peptide is configured to treat an autoimmune disease. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of Mokatoxin-1. In one embodiment, the therapeutic peptide comprises an amino acid sequence that is at least about 80% identical to an amino acid sequence of Mokatoxin-1. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 108. In one embodiment, the therapeutic peptide comprises an amino acid sequence that is at least about 80% identical to an amino acid sequence of SEQ ID NO: 108. In one embodiment, the therapeutic peptide comprises from about 15 to about 100 amino acids comprising from about 15 to about 34 amino acids identical to an amino acid sequence of SEQ ID NO: 108. Further provided herein is a method of treating an individual with an autoimmune disease, comprising administering an immunoglobulin fusion protein.

In one aspect of the disclosure, the therapeutic peptide of the immunoglobulin fusion protein is a neurotoxin. In one embodiment, the therapeutic peptide is configured to treat pain. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of neurotoxin mu-SLPTX-Ssm6a. In one embodiment, the therapeutic peptide comprises an amino acid sequence that is at least about 80% identical to an amino acid sequence of neurotoxin mu-SLPTX-Ssm6a. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 109. In one embodiment, the therapeutic peptide comprises an amino acid sequence that is at least about 80% identical to an amino acid sequence of SEQ ID NO: 109. In one embodiment, the therapeutic peptide comprises from about 15 to about 200 amino acids comprising from about 15 to about 46 amino acids identical to an amino acid sequence of SEQ ID NO: 109. Further provided herein is a method of treating an individual with pain, comprising administering an immunoglobulin fusion protein.

In one aspect of the disclosure, the therapeutic peptide of the immunoglobulin fusion protein comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of kappa-theraphotoxin-Tb1a. In one embodiment, the therapeutic peptide comprises an amino acid sequence that is at least about 80% identical to an amino acid sequence of kappa-theraphotoxin-Tb1a. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 110. In one embodiment, the therapeutic peptide comprises an amino acid sequence that is at least about 80% identical to an amino acid sequence of SEQ ID NO: 110. In one embodiment, the therapeutic peptide comprises from about 15 to about 100 amino acids comprising from about 15 to about 33 amino acids identical to an amino acid sequence of SEQ ID NO: 110. Further provided herein is a method of treating an individual with pain, comprising administering an immunoglobulin fusion protein.

In one aspect of the disclosure, the therapeutic peptide of the immunoglobulin fusion protein comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of mambalign-1. In one embodiment, the therapeutic peptide comprises an amino acid sequence that is at least about 80% identical to an amino acid sequence of mambalign-1. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 111. In one embodiment, the therapeutic peptide comprises an amino acid sequence that is at least about 80% identical to an amino acid sequence of SEQ ID NO: 111. In one embodiment, the therapeutic peptide comprises from about 15 to about 150 amino acids comprising from about 15 to about 57 amino acids identical to an amino acid sequence of SEQ ID NO: 111. Further provided herein is a method of treating an individual with pain, comprising administering an immunoglobulin fusion protein.

In one aspect of the disclosure, the therapeutic peptide of the immunoglobulin fusion protein is a hormone belonging to the insulin super family. In one embodiment, the therapeutic peptide is configured to treat a patient with heart failure. In one embodiment, the therapeutic peptide is configured to treat a patient with fibrosis. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of prorelaxin or relaxin. In one embodiment, the therapeutic peptide comprises an amino acid sequence that is at least about 80% identical to an amino acid sequence of prorelaxin or relaxin. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 99. In one embodiment, the therapeutic peptide comprises an amino acid sequence that is at least about 80% identical to an amino acid sequence of SEQ ID NO: 99. In one embodiment, the therapeutic peptide comprises from about 15 to about 200 amino acids comprising from about 15 to about 161 amino acids identical to an amino acid sequence of SEQ ID NO: 99. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 100. In one embodiment, the therapeutic peptide comprises an amino acid sequence that is at least about 80% identical to an amino acid sequence of SEQ ID NO: 100. In one embodiment, the therapeutic peptide comprises from about 15 to about 300 amino acids comprising from about 15 to about 185 amino acids identical to an amino acid sequence of SEQ ID NO: 100. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 101. In one embodiment, the therapeutic peptide comprises an amino acid sequence that is at least about 80% identical to an amino acid sequence of SEQ ID NO: 101. In one embodiment, the therapeutic peptide comprises from about 15 to about 200 amino acids comprising from about 15 to about 120 amino acids identical to an amino acid sequence of SEQ ID NO: 101. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 102. In one embodiment, the therapeutic peptide comprises an amino acid sequence that is at least about 80% identical to an amino acid sequence of SEQ ID NO: 102. In one embodiment, the therapeutic peptide comprises from about 15 to about 200 amino acids comprising from about 15 to about 88 amino acids identical to an amino acid sequence of SEQ ID NO: 102. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 103. In one embodiment, the therapeutic peptide comprises an amino acid sequence that is at least about 80% identical to an amino acid sequence of SEQ ID NO: 103. In one embodiment, the therapeutic peptide comprises from about 15 to about 200 amino acids comprising from about 15 to about 88 amino acids identical to an amino acid sequence of SEQ ID NO: 103. In one embodiment, the therapeutic peptide comprises an amino acid sequence that is about or at least about 50%, 60%, 70%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 104. In one embodiment, the therapeutic peptide comprises an amino acid sequence that is at least about 80% identical to an amino acid sequence of SEQ ID NO: 104. In one embodiment, the therapeutic peptide comprises from about 15 to about 200 amino acids comprising from about 15 to about 74 amino acids identical to an amino acid sequence of SEQ ID NO: 104. Further provided herein is a method of treating an individual with heart failure, comprising administering an immunoglobulin fusion protein.

Further provided herein is a first genetic construct comprising nucleic acids encoding the first immunoglobulin region, the first therapeutic peptide, and the connecting peptide. Further provided herein is a second genetic construct comprising nucleic acids encoding the second immunoglobulin region. Further provided herein is a first expression vector comprising the first genetic construct. Further provided herein is a second expression vector comprising the second genetic construct. Further provided herein is a mammalian expression host comprising the first expression vector. Further provided herein is a mammalian expression host comprising the second expression vector. Further provided herein is a method of producing an immunoglobulin fusion protein comprising: transfecting the first and/or the second expression vector transiently in a mammalian cell culture; growing the cell culture in an expression medium at a controlled temperature and percentage CO₂; and harvesting the secreted immunoglobulin fusion protein. In one embodiment, the method further comprises purifying the immunoglobulin fusion protein.

In one embodiment, the immunoglobulin fusion protein father comprises a second therapeutic peptide. In one embodiment, the second therapeutic peptide is attached to the first immunoglobulin region. In one embodiment, the immunoglobulin fusion protein further comprises a second immunoglobulin region. In one embodiment, the second therapeutic peptide is attached to the second immunoglobulin region. Further provided herein is a genetic construct comprising nucleic acids encoding the first immunoglobulin region and the first therapeutic peptide. Further provided herein is a genetic construct comprising nucleic acids encoding the first immunoglobulin region, the first therapeutic peptide, and the second therapeutic peptide. Further provided herein is a genetic construct comprising nucleic acids encoding the second immunoglobulin region and the second therapeutic peptide. Further provided herein is a host cell comprising any genetic construct disclosed herein. Further provided herein is a method of producing an immunoglobulin fusion protein, the method comprising culturing any host cell disclosed herein, under conditions wherein polynucleotides are expressed from the nucleic acids, thereby producing an immunoglobulin fusion protein.

Further provided herein are pharmaceutical compositions comprising any immunoglobulin fusion protein disclosed herein. In one embodiment, the pharmaceutical composition further comprises a pharmaceutically acceptable excipient. Further provided herein are methods of treating a disease or condition in a subject in need thereof, the method comprising administering to the subject a therapeutically effective amount of any immunoglobulin fusion protein disclosed herein.

### BRIEF DESCRIPTION OF THE DRAWINGS

The foregoing summary, as well as the following detailed description of the disclosure, will be better understood when read in conjunction with the appended figures. It should be understood, however, that the disclosure is not limited to the precise examples shown. It is emphasized that, according to common practice, the various features of the drawings are not to-scale. On the contrary, the dimensions of the various features are arbitrarily expanded or reduced for clarity. Included in the drawings are the following figures.

FIG. 1 depicts a graph of the activities of exendin-4 and trastuzumab(NL)-exendin-4 to activate GLP-1R.

FIG. 2 depicts a graph of the activities of exendin-4 and trastuzumab(NL, GGGGS)-ZP1 to activate GLP-1R.

FIG. 3 depicts a graph of the activities of trastuzumab (NL)-ZP1 to activate GCGR.

FIG. 4 depicts a graph of the activities of exendin-4 and trastuzumab(NL, GGGGS)-ZPCEX to activate GLP-1R.

FIG. 5 depicts a graph of the activities of trastuzumab (NL)-ZP1CEX to activate GCGR.

FIG. 6 depicts a graph of the activities of hLeptin, trastuzumab(CDR3H) Leptin, and trastuzumab(CDR3H) Leptin/trastuzumab(NL, GGGGS)-ZPCEX to activate leptin receptor.

FIG. 7 depicts a graph of the activities of exendin-4 and trastuzumab(CDR3H) Leptin/trastuzumab(NL, GGGGS)-ZPCEX to activate GLP-1R.

FIG. 8 depicts a graph of the activities of ZP2-DA and trastuzumab (NL)-ZP1CEX/trastuzumab (CDR)-leptin to activate GCGR.

FIG. 9 depicts a graph of the activities of exendin-4 and palivizumab (NL, GGGGS)-ZP1CEX to activate GLP-1R.

FIG. 10 depicts a graph of the activities of ZP2-DA and palivizumab (NL)-ZP1CEX to activate GCGR.

FIG. 11 depicts a graph of the activities of exendin-4 and palivizumab (NH, GGGGS)-ZP1CEX to activate GLP-1R.

FIG. 12 depicts a graph of the activities of ZP2-DA and palivizumab (NH)-ZP1CEX to activate GCGR.

FIGS. 13A and 13B depict graphs of the activities of palivizumab(NH, CEXGGGGS)-relaxin2(single) fusion proteins to activate relaxin receptors LGR7 and LGR8.

FIG. 14 depicts a graph of the activities of exendin-4 and trastuzumab(NL, GGGGS)-oxyntomodulin to activate GLP-1R.

FIG. 15 depicts a graph of the activity of trastuzumab (NL)-oxyntomodulin to activate GCGR.

FIGS. 16A-16K provide SDS-PAGE gels of purified palivizumab heavy chain relaxin fusion proteins expressed with palivizumab light chain.

FIGS. 17A and 17B provide SDS-PAGE gels of purified palivizumab heavy chain exendin-4 fusion proteins expressed with palivizumab light chain glucagon fusion proteins.

FIG. 18 provides a SDS-PAGE gel of purified palivizumab heavy chain ZP1 fusion protein expressed with palivizumab light chain.

FIGS. 19A and 19B provide SDS-PAGE gels of purified palivizumab heavy chain GLP2 fusion proteins expressed with palivizumab light chain.

FIG. 20 provides a graph of palivizumab heavy chain relaxin2 (single) fusion protein concentration versus time in a pharmacokinetic rat study.

FIG. 21 provides interpubic ligament length versus fusion protein dosage for mice treated with palivizumab heavy chain relaxin2 (single) fusion proteins.

FIG. 22 provides a graph of glucose measurements versus time for a pharmacodynamic study of palivizumab fusion proteins in mice.

FIGS. 23A and 23B depict graphs of the activities of palivizumab(NH, EAAAK)-relaxin(dual) fusion proteins to activate relaxin receptors LGR7 and LGR8.

FIGS. 24A and 24B provide graphs of palivizumab heavy chain relaxin (dual) fusion protein concentration in subcutaneously and intravenously treated rats in a pharmacokinetic study.

FIG. 25 provides interpubic ligament length versus fusion protein dosage for mice treated with palivizumab heavy chain relaxin (dual) fusion proteins.

### DETAILED DESCRIPTION OF THE INVENTION

Disclosed herein are amino-terminal immunoglobulin fusion proteins and methods of producing such immunoglobulin fusion proteins. Further provided herein are methods of treatment using said immunoglobulin fusion proteins. According to one feature of the subject matter described herein, an amino-terminal immunoglobulin fusion protein comprises (a) an immunoglobulin region; and (b) a therapeutic peptide connected to the amino terminus of the immunoglobulin region. The therapeutic peptide may be connected to the immunoglobulin region with a connecting peptide. In some embodiments, the immunoglobulin fusion protein further comprises one or more linker peptides. In some embodiments, the immunoglobulin fusion protein further comprises one or more protease cleavage sites. In some embodiments, the therapeutic peptide comprises one or more internal linker peptides.

According to another feature of the subject matter described herein, the amino-terminal immunoglobulin fusion protein further comprises a second immunoglobulin region. The second immunoglobulin region may comprise a single immunoglobulin domain or portion thereof, for example, a light chain or heavy chain domain. The second immunoglobulin region may be connected to a non-immunoglobulin region, forming a second immunoglobulin fusion. The non-immunoglobulin region may comprise a second therapeutic peptide. In some embodiments, the second therapeutic peptide further comprises an internal linker. The non-immunoglobulin region may further comprise one or more extender peptides, linker peptides, and/or proteolytic cleavage sites. In some embodiments, the first immunoglobulin region comprises amino acids from an immunoglobulin light chain. In some embodiments, the first immunoglobulin region comprises amino acids from an immunoglobulin heavy chain. In some embodiments, the second immunoglobulin region comprises amino acids from an immunoglobulin light chain. In some embodiments, the second immunoglobulin region comprises amino acids from an immunoglobulin heavy chain. The first immunoglobulin region and the second immunoglobulin region may be connected by one or more disulfide bonds or peptide linkers.

Further disclosed herein are dual immunoglobulin fusion proteins comprising two or more therapeutic peptides attached to an immunoglobulin region, wherein at least one therapeutic peptide is attached the amino terminus of the immunoglobulin region. A second therapeutic peptide may be connected to or inserted into the immunoglobulin region. A therapeutic peptide may replace at least a portion of the immunoglobulin region. In some embodiments, a therapeutic peptide comprises one portion of a therapeutic peptide and one or more portions of a second therapeutic peptide. In some embodiments, a therapeutic peptide comprises one portion of a therapeutic peptide, an internal linker, and a second portion of a therapeutic peptide, where both portions are derived from amino acids comprising the same therapeutic peptide. In some embodiments, a therapeutic peptide comprises an internal linker. In some embodiments, a therapeutic peptide comprises a protease cleavage site.

Exemplary amino-terminal immunoglobulin fusion proteins are depicted in Formulas I-XXXII, wherein T is a therapeutic peptide or a portion of a therapeutic peptide, C is a connecting peptide, A is an immunoglobulin region, P is a protease site, L is a linker, and I is an internal linker.

| Formula | Immunoglobulin fusion protein |
|---|---|
| I | T¹-A¹ |
| II | T¹-C-A¹ |
| III | T¹-C-P¹-A¹ |
| IV | T¹-P¹-C-A¹ |
| V | T¹-L¹-I-L²-T²-A¹ |
| VI | T¹-L¹-I-L²-T²-C-A¹ |
| VII | T¹-L¹-T²-L²-T³-A¹ |
| VIII | T¹-L¹-T²-L²-T³-C-A¹ |
| IX | T¹-P¹-I-P²-T²-A¹ |
| X | T¹-P¹-I-P²-T²-C-A¹ |
| XI | T¹-P¹-T²-P²-T³-A¹ |
| XII | T¹-P¹-T²-P²-T³-C-A¹ |
| XIII | T¹-P¹-L¹-I-L²-P²-T²-A¹ |
| XIV | T¹-P¹-L¹-I-L²-P²-T²-C-A¹ |
| XV | T¹-P¹-L¹-T²-L²-P²-T³-A¹ |
| XVI | T¹-P¹-L¹-T²-L²-P²-T³-C-A¹ |
| XVII | T¹-L¹-P¹-T²-A¹ |
| XVIII | T¹-P¹-L¹-T²-A¹ |
| XIX | T¹-P¹-L¹-T²-C-A¹ |
| XX | T¹-P¹-I-P²-P³-T²-A¹ |
| XXI | T¹-P¹-I-P²-P³-T²-A¹ |
| XXII | T¹-P¹-I-T²-A¹ |
| XXIII | T¹-P¹-I-T²-C-A¹ |
| XXIV | T¹-P¹-L-P²-P³-T²-A¹ |
| XXV | T¹-P¹-L-P²-P³-T²-C-A¹ |
| XXVI | T¹-P¹-T²-P²-P³-T³-A¹ |
| XXVII | T¹-P¹-T²-P²-P³-T³-C-A¹ |
| XXVIII | T¹-L-T²-A¹ |
| XXIX | T¹-L-T²-C-A¹ |
| XXX | T¹-I-T²-A¹ |
| XXXI | T¹-I-T²-C-A¹ |
| XXXII | T¹-P-T²-C-A¹ |

Further disclosed herein are methods of treating a disease or condition in a subject in need thereof. Generally, the method comprises administering to the subject an amino-terminal immunoglobulin fusion protein comprising a therapeutic peptide attached to the amino terminus of an immunoglobulin region. In some embodiments, an immunoglobulin fusion protein having the formula of I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, XXXII, or any modification, portions, or additions thereof is administered to a patient. In some embodiments, one or more of the immunoglobulin fusion proteins I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, XXXI, or XXXII, further comprising a second immunoglobulin region, is administered to a patient.

Further disclosed herein are methods of improving the delivery of a therapeutic peptide. The methods may involve generation of an amino-terminal immunoglobulin fusion protein from a genetic construct. In some embodiments, the immunoglobulin fusion protein is recombinantly produced from a genetic construct encoding the immunoglobulin fusion protein. In some embodiments, the construct is expressed in vitro using standard mammalian cell culture techniques. In some embodiments, one construct encoding a therapeutic peptide connected to the amino-terminus of a first immunoglobulin region is co-expressed with a second construct comprising a second immunoglobulin region, to produce a recombinant immunoglobulin fusion protein. In some embodiments, a construct encoding a protease is co-expressed with an immunoglobulin fusion protein. The method may further comprise generating immunoglobulin genetic fusion constructs comprising one or more connecting peptides, internal linkers, linkers, extender peptides, and/or proteolytic cleavage sites.

Before the present methods and compositions are described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims. Examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

### Amino-Terminal Immunoglobulin Fusion Proteins

The amino-terminal immunoglobulin fusion proteins disclosed herein comprise one or more immunoglobulin regions and one or more therapeutic peptides, wherein a first therapeutic peptide is connected to an amino-terminus of a first immunoglobulin region. The immunoglobulin region may be any portion, in part or whole, of an immunoglobulin. The immunoglobulin may be from a mammalian source. The immunoglobulin may be a chimeric immunoglobulin. The immunoglobulin region may be derived in whole or in part from an engineered immunoglobulin or recombinant immunoglobulin. The immunoglobulin may be from a humanized, human engineered or fully human immunoglobulin. The mammalian immunoglobulin may be a bovine immunoglobulin. The mammalian immunoglobulin may be a human immunoglobulin. The mammalian immunoglobulin may be a murine immunoglobulin. The mammalian immunoglobulin may be a non-human primate immunoglobulin. The immunoglobulin may be an avian immunoglobulin. The immunoglobulin may be a shark immunoglobulin.

The immunoglobulin region may comprise an entire immunoglobulin molecule or any polypeptide comprising fragment of an immunoglobulin including, but not limited to, heavy chain, light chain, variable domain, constant domain, complementarity determining region (CDR), framework region, fragment antigen binding (Fab) region, Fab′, F(ab′)2, F(ab′)3, Fab′, fragment crystallizable (Fc) region, single chain variable fragment (scFV), di-scFv, single domain immunoglobulin, trifunctional immunoglobulin, chemically linked F(ab′)2, and any portion or combination thereof. In some embodiments, an immunoglobulin heavy chain may comprise an entire heavy chain or a portion of a heavy chain. For example, a variable domain or region thereof derived from a heavy chain may be referred to as a heavy chain or a region of a heavy chain. In some embodiments, an immunoglobulin light chain may comprise an entire light chain or a portion of a light chain. For example, a variable domain or region thereof derived from a light chain may be referred to as a light chain or a region of a light chain. The immunoglobulin region may be bispecific or trispecific. A single domain immunoglobulin includes, but is not limited to, a single monomeric variable immunoglobulin domain. The single domain immunoglobulin may be a shark variable new antigen receptor immunoglobulin fragment (VNAR). The immunoglobulin may be derived from any type known to one of skill in the art including, but not limited to, IgA, IgD, IgE, IgG, IgM, IgY, IgW. The immunoglobulin region may be a glycoprotein. The immunoglobulin region may comprise one or more functional units, including but not limited to, 1, 2, 3, 4, and 5 units. The immunoglobulin region may comprise one or more units connected by one or more disulfide bonds. The immunoglobulin region may comprise one or more units connected by a peptide linker, for example, a scFv immunoglobulin. The immunoglobulin may be a recombinant immunoglobulin including immunoglobulins with amino acid mutations, substitutions, and/or deletions. The immunoglobulin may be a recombinant immunoglobulin comprising chemical modifications. The immunoglobulin may comprise a whole or part of an immunoglobulin-drug conjugate. The immunoglobulin may comprise a small molecule. The immunoglobulin may comprise a whole or part of an immunoglobulin-drug conjugate comprising a small molecule. Examples of an immunoglobulin-drug conjugated include, but are not limited to, Brentuximab vedotin (SGN35), Trastuzumab emtansine (T-DM1), Inotuzumab ozogamicin (CMC-544), Gemtuzumab ozogamicin, SAR3419, RG-7596/DCDS4501A, Pinatuzumab vedotin (RG-7593/DCDT 2980S), Glembatumumab vedotin (CDX-011), Lorvotuzumab mertansine (IMGN901), PSMA-ADC, BT-062, ABT-414, Milatuzumab doxorubicin (IMMU-110), IMMU-132 (hRS7-SN38), Labetuzumab-SN-38 (IMMU-130), Epratuzumab-SN-38, IMGN-853, RG-7458/DMUC 5754 A, RG-7636, RG-7450/DSTP 3086 S, RG-7600, RG-7598, RG-7599/DNIB 0600 A, SGN-CD19A, SGN-CD33A (EC-mAb), SGN-75, SGN CD70 A, PF-0626350, Vorsetuzumab mafodotin, ASG-5ME, ASG-22ME, ASG-22CE, AGS-16M8F, ASG-15ME, MLN-0264, SAR-566658, AMG-172, AMG-595, BAY-94-9343, BAY-79-4620, SC16LD6.5, SGN-LIV1-A, MDX-1203, BIIB015, HuMax-TF-ADC, and ARX788.

The immunoglobulin fusion protein may comprise an amino acid sequence that is based on or derived from any one of SEQ ID NOs: 42-74, 192-221. The immunoglobulin fusion protein may comprise an amino acid sequence that is at least about 50% homologous to any one of SEQ ID NOs: 42-74, 192-221. The immunoglobulin fusion protein may comprise an amino acid sequence that is at least about 60%, 65%, 70%, 75%, 80%, 85%, 90%, 95%, or 97% homologous to any one of SEQ ID NOs 42-74, 192-221. The immunoglobulin fusion protein may comprise an amino acid sequence that is at least about 70% homologous to any one of SEQ ID NOs: 42-74, 192-221. The immunoglobulin fusion protein may comprise an amino acid sequence that is at least about 80% homologous to any one of SEQ ID NOs: 42-74, 192-221. The immunoglobulin fusion protein may comprise an amino acid sequence that is at least about 50% identical to any one of SEQ ID NOs: 42-74, 192-221. The immunoglobulin fusion protein may comprise an amino acid sequence that is at least about 60%, 65%, 70%, 75%, 80%, 85%, 90%, 95%, or 97% identical to any one of SEQ ID NOs 42-74, 192-221. The immunoglobulin fusion protein may comprise an amino acid sequence that is at least about 70% identical to any one of SEQ ID NOs: 42-74, 192-221. The immunoglobulin fusion protein may comprise an amino acid sequence that is at least about 80% identical to any one of SEQ ID NOs: 42-74, 192-221. The immunoglobulin fusion protein may comprise an amino acid sequence that is 100% identical to any one of SEQ ID NOs: 42-74, 192-221. In some embodiments, the immunoglobulin fusion protein comprises an amino acid sequence that is at least about 50%, 55%, 60%, 65%, 70%, 75%, 80%, 85%, 90%, 95%, or 97% homologous to an amino acid sequence of any one of SEQ ID NOs: 42-74, 192-221. In some embodiments, the immunoglobulin fusion protein comprises an amino acid sequence that is at least about 50%, 55%, 60%, 65%, 70%, 75%, 80%, 85%, 90%, 95%, or 97% identical to an amino acid sequence of any one of SEQ ID NOs: 42-74, 192-221.

The immunoglobulin fusion protein may comprise an amino acid sequence comprising 10, 20, 30, 40, 50, 60, 70, 80, 90, 100 or more amino acids based on or derived from any one of SEQ ID NOs: 42-74, 192-221. The immunoglobulin fusion protein may comprise an amino acid sequence comprising 125, 150, 175, 200, 225, 250, 275, 300, 325, 350, 375, 400, 425, 450, 475, 450, 500 or more amino acids based on or derived from any one of SEQ ID NOs: 42-74, 192-221. The immunoglobulin fusion protein may comprise an amino acid sequence comprising 10 or more amino acids based on or derived from any one of SEQ ID NOs: 42-74, 192-221. The immunoglobulin fusion protein may comprise an amino acid sequence comprising 50 or more amino acids based on or derived from any one of SEQ ID NOs: 42-74, 192-221. The immunoglobulin fusion protein may comprise an amino acid sequence comprising 100 or more amino acids based on or derived from any one of SEQ ID NOs: 42-74, 192-221. The immunoglobulin fusion protein may comprise an amino acid sequence comprising 200 or more amino acids based on or derived from any one of SEQ ID NOs: 42-74, 192-221. The amino acids may be consecutive. Alternatively, or additionally, the amino acids are nonconsecutive. In some embodiments, the immunoglobulin fusion protein may comprise amino acids derived from any one of SEQ ID NOs: 42-74, 192-221 and amino acids not derived from any one of SEQ ID NOs: 42-74, 192-221. In some embodiments, the immunoglobulin fusion protein may comprise amino acids derived from one or more of SEQ ID NOs: 42-74, 192-221 and amino acids not derived from any one of SEQ ID NOs: 42-74, 192-221. In some embodiments, the immunoglobulin fusion protein comprises amino acids derived from 1, 2, 3, 4, 5, 6, 7, 8, 9, 10 or more of SEQ ID NOs: 42-74, 192-221.

The immunoglobulin fusion protein may be encoded by a nucleotide sequence that is based on or derived from any one of SEQ ID NOs: 9-41, 161-191, 265. The immunoglobulin fusion protein may be encoded by a nucleotide sequence that is at least about 50% homologous to any one of SEQ ID NOs: 9-41, 161-191, 265. The immunoglobulin fusion protein may be encoded by a nucleotide sequence that is at least about 60%, 65%, 70%, 75%, 80%, 85%, 90%, 95%, or 97% homologous to any one of SEQ ID NOs: 9-41, 161-191, 265. The immunoglobulin fusion protein may be encoded by a nucleotide sequence that is at least about 70% homologous to any one of SEQ ID NOs: 9-41, 161-191, 265. The immunoglobulin fusion protein may be encoded by a nucleotide sequence that is at least about 80% homologous to any one of SEQ ID NOs: 9-41, 161-191, 265. The immunoglobulin fusion protein may be encoded by a nucleotide sequence that is at least about 50% identical to any one of SEQ ID NOs: 9-41, 161-191, 265. The immunoglobulin fusion protein may be encoded by a nucleotide sequence that is at least about 60%, 65%, 70%, 75%, 80%, 85%, 90%, 95%, or 97% identical to any one of SEQ ID NOs: 9-41, 161-191, 265. The immunoglobulin fusion protein may be encoded by a nucleotide sequence that is at least about 70% identical to any one of SEQ ID NOs: 9-41, 161-191, 265. The immunoglobulin fusion protein may be encoded by a nucleotide sequence that is at least about 80% identical to any one of SEQ ID NOs: 9-41, 161-191, 265. The immunoglobulin fusion protein may be encoded by a nucleotide sequence that is 100% identical to any one of SEQ ID NOs: 9-41, 161-191, 265. In some embodiments, the immunoglobulin fusion protein is encoded by a nucleotide sequence that is at least about 50%, 55%, 60%, 65%, 70%, 75%, 80%, 85%, 90%, 95%, or 97% homologous to an amino acid sequence of any one of SEQ ID NOs: 9-41, 161-191, 265. In some embodiments, the immunoglobulin fusion protein is encoded by a nucleotide sequence that is at least about 50%, 55%, 60%, 65%, 70%, 75%, 80%, 85%, 90%, 95%, or 97% identical to an amino acid sequence of any one of SEQ ID NOs: 9-41, 161-191, 265.

The immunoglobulin fusion protein may be encoded by a nucleotide sequence comprising 10, 20, 30, 40, 50, 60, 70, 80, 90, 100 or more nucleotides based on or derived from any one of SEQ ID NOs: 9-41, 161-191, 265. The immunoglobulin fusion protein may be encoded by a nucleotide sequence comprising 125, 150, 175, 200, 225, 250, 275, 300, 325, 350, 375, 400, 425, 450, 475, 450, 500 or more nucleotides based on or derived from any one of SEQ ID NOs: 9-41, 161-191, 265. The immunoglobulin fusion protein may be encoded by a nucleotide sequence comprising 600, 650, 700, 750, 800, 850, 900, 950, 1000 or more nucleotides based on or derived from any one of SEQ ID NOs: 9-41, 161-191, 265. The immunoglobulin fusion protein may be encoded by a nucleotide sequence comprising 1100, 1200, 1300, 1400, 1500 or more nucleotides based on or derived from any one of SEQ ID NOs: 9-41, 161-191, 265. The immunoglobulin fusion protein may be encoded by a nucleotide sequence comprising 100 or more nucleotides based on or derived from any one of SEQ ID NOs: 9-41, 161-191, 265. The immunoglobulin fusion protein may be encoded by a nucleotide sequence comprising 500 or more nucleotides based on or derived from any one of SEQ ID NOs: 9-41, 161-191, 265. The immunoglobulin fusion protein may be encoded by a nucleotide sequence comprising 1,000 or more nucleotides based on or derived from any one of SEQ ID NOs: 25-44. The immunoglobulin fusion protein may be encoded by a nucleotide sequence comprising 1,300 or more nucleotides based on or derived from any one of SEQ ID NOs: 9-41, 161-191, 265. The nucleotides may be consecutive. Alternatively, or additionally, the nucleotides are nonconsecutive. In some embodiments, the immunoglobulin fusion protein is encoded by a nucleotide sequence comprising nucleotides derived from any one of SEQ ID NOs: 9-41, 161-191, 265 and nucleotides not derived from any one of SEQ ID NOs: 9-41, 161-191, 265. In some embodiments, the immunoglobulin fusion protein is encoded by a nucleotide sequence comprising nucleotides derived from one or more of SEQ ID NOs: 25-44 and nucleotides not derived from any one of SEQ ID NOs: 9-41, 161-191, 265. In some embodiments, the immunoglobulin fusion protein is encoded by a nucleotide sequence derived from 1, 2, 3, 4, 5, 6, 7, 8, 9, 10 or more of SEQ ID NOs: 9-41, 161-191, 265.

Further disclosed herein are nucleotide constructs comprising a nucleotide sequence that is based on or derived from any one of SEQ ID NOs: 9-41, 161-191, 265. The nucleotide construct may be a plasmid for expression in a host cell. For example, a mammalian or bacterial expression plasmid. In some embodiments, the construct comprises a nucleotide sequence that is at least about 50% homologous to any one of SEQ ID NOs: 9-41, 161-191, 265. In some embodiments, the construct comprises a nucleotide sequence that is at least about 60%, 65%, 70%, 75%, 80%, 85%, 90%, 95%, or 97% homologous to any one of SEQ ID NOs: 9-41, 161-191, 265. In some embodiments, the construct comprises a nucleotide sequence that is at least about 70% homologous to any one of SEQ ID NOs: 9-41, 161-191, 265. In some embodiments, the construct comprises a nucleotide sequence that is at least about 80% homologous to any one of SEQ ID NOs: 9-41, 161-191, 265. In some embodiments, the construct comprises a nucleotide sequence that is at least about 50% identical to any one of SEQ ID NOs: 9-41, 161-191, 265. In some embodiments, the construct comprises a nucleotide sequence that is at least about 60%, 65%, 70%, 75%, 80%, 85%, 90%, 95%, or 97% identical to any one of SEQ ID NOs: 9-41, 161-191, 265. In some embodiments, the construct comprises a nucleotide sequence that is at least about 70% identical to any one of SEQ ID NOs: 9-41, 161-191, 265. In some embodiments, the construct comprises a nucleotide sequence that is at least about 80% identical to any one of SEQ ID NOs: 9-41, 161-191, 265. In some embodiments, the construct comprises a nucleotide sequence that is 100% identical to any one of SEQ ID NOs: 9-41, 161-191, 265. In some embodiments, the construct comprises a nucleotide sequence that is at least about 50%, 55%, 60%, 65%, 70%, 75%, 80%, 85%, 90%, 95%, or 97% homologous to an amino acid sequence of any one of SEQ ID NOs: 9-41, 161-191, 265. In some embodiments, the construct comprises a nucleotide sequence that is at least about 50%, 55%, 60%, 65%, 70%, 75%, 80%, 85%, 90%, 95%, or 97% identical to an amino acid sequence of any one of SEQ ID NOs: 9-41, 161-191, 265.

### Amino-Terminal Immunoglobulin Light Chain Fusions

In one feature of the invention, provided herein is an immunoglobulin fusion protein comprising a therapeutic peptide connected to the amino-terminus of a region of an immunoglobulin light chain, wherein the immunoglobulin fusion is referred to herein as an immunoglobulin light chain fusion. In some embodiments, the immunoglobulin fusion protein further comprises one or more regions of an immunoglobulin heavy chain, wherein the immunoglobulin light chain fusion is connected to the one or more regions of an immunoglobulin heavy chain by disulfide bonds or a connecting peptide. In some embodiments, the therapeutic peptide comprises one or more regions of a therapeutic peptide. In some embodiments, the therapeutic peptide comprises two regions of a therapeutic peptide connected by an internal linker. In some embodiments, the therapeutic peptide comprises a protease cleavage site.

The immunoglobulin light chain fusion may comprise an amino acid sequence that is based on or derived from any one of SEQ ID NOs: 5, 7, 42, 45-49, 51-74, 193, 194, 199, 200, 214, 215, 221. The immunoglobulin light chain fusion may comprise an amino acid sequence that is at least about 50% homologous to any one of SEQ ID NOs: 5, 7, 42, 45-49, 51-74, 193, 194, 199, 200, 214, 215, 221. The immunoglobulin light chain fusion may comprise an amino acid sequence that is at least about 60%, 65%, 70%, 75%, 80%, 85%, 90%, 95%, or 97% homologous to any one of SEQ ID NOs: 5, 7, 42, 45-49, 51-74, 193, 194, 199, 200, 214, 215, 221. The immunoglobulin light chain fusion may comprise an amino acid sequence that is at least about 70% homologous to any one of SEQ ID NOs: 5, 7, 42, 45-49, 51-74, 193, 194, 199, 200, 214, 215, 221. The immunoglobulin light chain fusion may comprise an amino acid sequence that is at least about 80% homologous to any one of SEQ ID NOs: 5, 7, 42, 45-49, 51-74, 193, 194, 199, 200, 214, 215, 221. The immunoglobulin light chain fusion may comprise an amino acid sequence that is at least about 50% identical to any one of SEQ ID NOs: 5, 7, 42, 45-49, 51-74, 193, 194, 199, 200, 214, 215, 221. The immunoglobulin light chain fusion may comprise an amino acid sequence that is at least about 60%, 65%, 70%, 75%, 80%, 85%, 90%, 95%, or 97% identical to any one of SEQ ID NOs: 5, 7, 42, 45-49, 51-74, 193, 194, 199, 200, 214, 215, 221. The immunoglobulin light chain fusion may comprise an amino acid sequence that is at least about 70% identical to any one of SEQ ID NOs: 5, 7, 42, 45-49, 51-74, 193, 194, 199, 200, 214, 215, 221. The immunoglobulin light chain fusion may comprise an amino acid sequence that is at least about 80% identical to any one of SEQ ID NOs: 5, 7, 42, 45-49, 51-74, 193, 194, 199, 200, 214, 215, 221. The immunoglobulin light chain fusion may comprise an amino acid sequence that is 100% identical to any one of SEQ ID NOs: 5, 7, 42, 45-49, 51-74, 193, 194, 199, 200, 214, 215, 221. The immunoglobulin heavy chain may comprise an amino acid sequence that is based on or derived from any one of SEQ ID NOs: 6, 8, 43-44, 50, 192, 195-198, 201-213, 216-220, 222, 266. The immunoglobulin heavy chain may comprise an amino acid sequence that is at least about 50% homologous to any one of SEQ ID NOs: 6, 8, 43-44, 50, 192, 195-198, 201-213, 216-220, 222, 266. The immunoglobulin heavy chain may comprise an amino acid sequence that is at least about 60%, 65%, 70%, 75%, 80%, 85%, 90%, 95%, or 97% homologous to any one of SEQ ID NOs: 6, 8, 43-44, 50, 192, 195-198, 201-213, 216-220, 222, 266. The immunoglobulin heavy chain may comprise an amino acid sequence that is at least about 70% homologous to any one of SEQ ID NOs: 6, 8, 43-44, 50, 192, 195-198, 201-213, 216-220, 222, 266. The immunoglobulin heavy chain may comprise an amino acid sequence that is at least about 80% homologous to any one of SEQ ID NOs: 6, 8, 43-44, 50, 192, 195-198, 201-213, 216-220, 222, 266. The immunoglobulin heavy chain may comprise an amino acid sequence that is at least about 50% identical to any one of SEQ ID NOs: 6, 8, 43-44, 50, 192, 195-198, 201-213, 216-220, 222, 266. The immunoglobulin heavy chain may comprise an amino acid sequence that is at least about 60%, 65%, 70%, 75%, 80%, 85%, 90%, 95%, or 97% identical to any one of SEQ ID NOs: 6, 8, 43-44, 50, 192, 195-198, 201-213, 216-220, 222, 266. The immunoglobulin heavy chain may comprise an amino acid sequence that is at least about 70% identical to any one of SEQ ID NOs: 6, 8, 43-44, 50, 192, 195-198, 201-213, 216-220, 222, 266. The immunoglobulin heavy chain may comprise an amino acid sequence that is at least about 80% identical to any one of SEQ ID NOs: 6, 8, 43-44, 50, 192, 195-198, 201-213, 216-220, 222, 266. The immunoglobulin heavy chain may comprise an amino acid sequence that is 100% identical to any one of SEQ ID NOs: 6, 8, 43-44, 50, 192, 195-198, 201-213, 216-220, 222, 266.

The immunoglobulin light chain fusion may comprise an amino acid sequence comprising 10, 20, 30, 40, 50, 60, 70, 80, 90, 100 or more amino acids based on or derived from any one of SEQ ID NOs: 5, 7, 42, 45-49, 51-74, 193, 194, 199, 200, 214, 215, 221. The immunoglobulin light chain fusion may comprise an amino acid sequence comprising 125, 150, 175, 200, 225, 250, 275, 300, 325, 350, 375, 400, 425, 450, 475, 450, 500 or more amino acids based on or derived from any one of SEQ ID NOs: 5, 7, 42, 45-49, 51-74, 193, 194, 199, 200, 214, 215, 221. The immunoglobulin light chain fusion may comprise an amino acid sequence comprising 10 or more amino acids based on or derived from any one of SEQ ID NOs: 5, 7, 42, 45-49, 51-74, 193, 194, 199, 200, 214, 215, 221. The immunoglobulin light chain fusion may comprise an amino acid sequence comprising 50 or more amino acids based on or derived from any one of SEQ ID NOs: 5, 7, 42, 45-49, 51-74, 193, 194, 199, 200, 214, 215, 221. The immunoglobulin light chain fusion may comprise an amino acid sequence comprising 100 or more amino acids based on or derived from any one of SEQ ID NOs: 5, 7, 42, 45-49, 51-74, 193, 194, 199, 200, 214, 215, 221. The immunoglobulin light chain fusion may comprise an amino acid sequence comprising 200 or more amino acids based on or derived from any one of SEQ ID NOs: 5, 7, 42, 45-49, 51-74, 193, 194, 199, 200, 214, 215, 221. The amino acids may be consecutive. Alternatively, or additionally, the amino acids are nonconsecutive. In some embodiments, the immunoglobulin light chain fusion may comprise amino acids derived from any one of SEQ ID NOs: 5, 7, 42, 45-49, 51-74, 193, 194, 199, 200, 214, 215, 221 and amino acids not derived from any one of SEQ ID NOs: 5, 7, 42, 45-49, 51-74, 193, 194, 199, 200, 214, 215, 221. In some embodiments, the immunoglobulin light chain fusion may comprise amino acids derived from one or more of SEQ ID NOs: 5, 7, 42, 45-49, 51-74, 193, 194, 199, 200, 214, 215, 221 and amino acids not derived from any one of SEQ ID NOs: 5, 7, 42, 45-49, 51-74, 193, 194, 199, 200, 214, 215, 221. In some embodiments, the immunoglobulin light chain fusion comprises amino acids derived from 1, 2, 3, 4, 5, 6, 7, 8, 9, 10 or more of SEQ ID NOs: 5, 7, 42, 45-49, 51-74, 193, 194, 199, 200, 214, 215, 221.

The immunoglobulin light chain fusion may be encoded by a nucleotide sequence that is based on or derived from any one of SEQ ID NOs: 1, 3, 9, 12-16, 18-41, 162, 163, 168, 169, 183, 184, 190. The immunoglobulin light chain fusion may be encoded by a nucleotide sequence that is at least about 50% homologous to any one of SEQ ID NOs: 1, 3, 9, 12-16, 18-41, 162, 163, 168, 169, 183, 184, 190. The immunoglobulin light chain fusion may be encoded by a nucleotide sequence that is at least about 60%, 65%, 70%, 75%, 80%, 85%, 90%, 95%, or 97% homologous to any one of SEQ ID NOs: 1, 3, 9, 12-16, 18-41, 162, 163, 168, 169, 183, 184, 190. The immunoglobulin light chain fusion may be encoded by a nucleotide sequence that is at least about 70% homologous to any one of SEQ ID NOs: 1, 3, 9, 12-16, 18-41, 162, 163, 168, 169, 183, 184, 190. The immunoglobulin light chain fusion may be encoded by a nucleotide sequence that is at least about 80% homologous to any one of SEQ ID NOs: 1, 3, 9, 12-16, 18-41, 162, 163, 168, 169, 183, 184, 190. The immunoglobulin light chain fusion may be encoded by a nucleotide sequence that is at least about 50% identical to any one of SEQ ID NOs: 1, 3, 9, 12-16, 18-41, 162, 163, 168, 169, 183, 184, 190. The immunoglobulin light chain fusion may be encoded by a nucleotide sequence that is at least about 60%, 65%, 70%, 75%, 80%, 85%, 90%, 95%, or 97% identical to any one of SEQ ID NOs: 1, 3, 9, 12-16, 18-41, 162, 163, 168, 169, 183, 184, 190. The immunoglobulin light chain fusion may be encoded by a nucleotide sequence that is at least about 70% identical to any one of SEQ ID NOs: 1, 3, 9, 12-16, 18-41, 162, 163, 168, 169, 183, 184, 190. The immunoglobulin light chain fusion may be encoded by a nucleotide sequence that is at least about 80% identical to any one of SEQ ID NOs: 1, 3, 9, 12-16, 18-41, 162, 163, 168, 169, 183, 184, 190. The immunoglobulin light chain fusion may be encoded by a nucleotide sequence that is 100% identical to any one of SEQ ID NOs: 1, 3, 9, 12-16, 18-41, 162, 163, 168, 169, 183, 184, 190.

The immunoglobulin light chain fusion may be encoded by a nucleotide sequence comprising 10, 20, 30, 40, 50, 60, 70, 80, 90, 100 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 125, 150, 175, 200, 225, 250, 275, 300, 325, 350, 375, 400, 425, 450, 475, 450, 500 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 600, 650, 700, 750, 800, 850, 900, 950, 1000 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 1100, 1200, 1300, 1400, 1500 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 100 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 500 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 1000 or more nucleotides based on or derived from any one of SEQ ID NOs: 1, 3, 9, 12-16, 18-41, 162, 163, 168, 169, 183, 184, 190. The immunoglobulin light chain fusion may be encoded by a nucleotide sequence comprising 1300 or more nucleotides based on or derived from any one of SEQ ID NOs: 1, 3, 9, 12-16, 18-41, 162, 163, 168, 169, 183, 184, 190. The nucleotides may be consecutive. Alternatively, or additionally, the nucleotides are nonconsecutive. In some embodiments, the immunoglobulin light chain fusion is encoded by a nucleotide sequence comprising nucleotides derived from any one of SEQ ID NOs: 1, 3, 9, 12-16, 18-41, 162, 163, 168, 169, 183, 184, 190 and nucleotides not derived from any one of SEQ ID NOs: 1, 3, 9, 12-16, 18-41, 162, 163, 168, 169, 183, 184, 190. In some embodiments, the immunoglobulin light chain fusion is encoded by a nucleotide sequence comprising nucleotides derived from one or more of SEQ ID NOs: 1, 3, 9, 12-16, 18-41, 162, 163, 168, 169, 183, 184, 190 and nucleotides not derived from any one of SEQ ID NOs: 1, 3, 9, 12-16, 18-41, 162, 163, 168, 169, 183, 184, 190. In some embodiments, the immunoglobulin light chain fusion is encoded by a nucleotide sequence derived from 1, 2, 3, 4, 5, 6, 7, 8, 9, 10 or more of SEQ ID NOs: 1, 3, 9, 12-16, 18-41, 162, 163, 168, 169, 183, 184, 190.

### Amino-Terminal Immunoglobulin Heavy Chain Fusions

In one feature of the invention, provided herein is an immunoglobulin fusion protein comprising a therapeutic peptide connected to the amino-terminus of a region of an immunoglobulin heavy chain, wherein the immunoglobulin fusion is referred to herein as an immunoglobulin heavy chain fusion. In some embodiments, the immunoglobulin fusion protein further comprises one or more regions of an immunoglobulin light chain, wherein the immunoglobulin heavy chain fusion is connected to the one or more regions of an immunoglobulin light chain by disulfide bonds or a connecting peptide. In some embodiments, the therapeutic peptide comprises one or more regions of a therapeutic peptide. In some embodiments, the therapeutic peptide comprises two regions of a therapeutic peptide connected by an internal linker. In some embodiments, the therapeutic peptide comprises a protease cleavage site.

The immunoglobulin heavy chain fusion may comprise an amino acid sequence that is based on or derived from any one of SEQ ID NOs: 6, 8, 43-44, 50, 192, 195-198, 201-213, 216-220, 222, 266. The immunoglobulin heavy chain fusion may comprise an amino acid sequence that is at least about 50% homologous to any one of SEQ ID NOs: 6, 8, 43-44, 50, 192, 195-198, 201-213, 216-220, 222, 266. The immunoglobulin heavy chain fusion may comprise an amino acid sequence that is at least about 60%, 65%, 70%, 75%, 80%, 85%, 90%, 95%, or 97% homologous to any one of SEQ ID NOs: 6, 8, 43-44, 50, 192, 195-198, 201-213, 216-220, 222, 266. The immunoglobulin heavy chain fusion may comprise an amino acid sequence that is at least about 70% homologous to any one of SEQ ID NOs: 6, 8, 43-44, 50, 192, 195-198, 201-213, 216-220, 222, 266. The immunoglobulin heavy chain fusion may comprise an amino acid sequence that is at least about 80% homologous to any one of SEQ ID NOs: 6, 8, 43-44, 50, 192, 195-198, 201-213, 216-220, 222, 266. The immunoglobulin heavy chain fusion may comprise an amino acid sequence that is at least about 50% identical to any one of SEQ ID NOs: 6, 8, 43-44, 50, 192, 195-198, 201-213, 216-220, 222, 266. The immunoglobulin heavy chain fusion may comprise an amino acid sequence that is at least about 60%, 65%, 70%, 75%, 80%, 85%, 90%, 95%, or 97% identical to any one of SEQ ID NOs: 6, 8, 43-44, 50, 192, 195-198, 201-213, 216-220, 222, 266. The immunoglobulin heavy chain fusion may comprise an amino acid sequence that is at least about 70% identical to any one of SEQ ID NOs: 6, 8, 43-44, 50, 192, 195-198, 201-213, 216-220, 222, 266. The immunoglobulin heavy chain fusion may comprise an amino acid sequence that is at least about 80% identical to any one of SEQ ID NOs: 6, 8, 43-44, 50, 192, 195-198, 201-213, 216-220, 222, 266. The immunoglobulin heavy chain fusion may comprise an amino acid sequence that is 100% identical to any one of SEQ ID NOs: 6, 8, 43-44, 50, 192, 195-198, 201-213, 216-220, 222, 266. The immunoglobulin light chain may comprise an amino acid sequence that is based on or derived from any one of SEQ ID NOs: 5, 7, 42, 45-49, 51-74, 193, 194, 199, 200, 214, 215, 221. The immunoglobulin light chain may comprise an amino acid sequence that is at least about 50% homologous to any one of SEQ ID NOs: 5, 7, 42, 45-49, 51-74, 193, 194, 199, 200, 214, 215, 221. The immunoglobulin light chain may comprise an amino acid sequence that is at least about 60%, 65%, 70%, 75%, 80%, 85%, 90%, 95%, or 97% homologous to any one of SEQ ID NOs: 5, 7, 42, 45-49, 51-74, 193, 194, 199, 200, 214, 215, 221. The immunoglobulin light chain may comprise an amino acid sequence that is at least about 70% homologous to any one of SEQ ID NOs: 5, 7, 42, 45-49, 51-74, 193, 194, 199, 200, 214, 215, 221. The immunoglobulin light chain may comprise an amino acid sequence that is at least about 80% homologous to any one of SEQ ID NOs: 5, 7, 42, 45-49, 51-74, 193, 194, 199, 200, 214, 215, 221. The immunoglobulin light chain may comprise an amino acid sequence that is at least about 50% identical to any one of SEQ ID NOs: 5, 7, 42, 45-49, 51-74, 193, 194, 199, 200, 214, 215, 221. The immunoglobulin light chain may comprise an amino acid sequence that is at least about 60%, 65%, 70%, 75%, 80%, 85%, 90%, 95%, or 97% identical to any one of SEQ ID NOs: 5, 7, 42, 45-49, 51-74, 193, 194, 199, 200, 214, 215, 221. The immunoglobulin light chain may comprise an amino acid sequence that is at least about 70% identical to any one of SEQ ID NOs: 5, 7, 42, 45-49, 51-74, 193, 194, 199, 200, 214, 215, 221. The immunoglobulin light chain may comprise an amino acid sequence that is at least about 80% identical to any one of SEQ ID NOs: 5, 7, 42, 45-49, 51-74, 193, 194, 199, 200, 214, 215, 221. The immunoglobulin light chain may comprise an amino acid sequence that is 100% identical to any one of SEQ ID NOs: 5, 7, 42, 45-49, 51-74, 193, 194, 199, 200, 214, 215, 221.

The immunoglobulin heavy chain fusion may comprise an amino acid sequence comprising 10, 20, 30, 40, 50, 60, 70, 80, 90, 100 or more amino acids based on or derived from any one of SEQ ID NOs: 6, 8, 43-44, 50, 192, 195-198, 201-213, 216-220, 222, 266. The immunoglobulin heavy chain fusion may comprise an amino acid sequence comprising 125, 150, 175, 200, 225, 250, 275, 300, 325, 350, 375, 400, 425, 450, 475, 450, 500 or more amino acids based on or derived from any one of SEQ ID NOs: 6, 8, 43-44, 50, 192, 195-198, 201-213, 216-220, 222, 266. The immunoglobulin heavy chain fusion may comprise an amino acid sequence comprising 10 or more amino acids based on or derived from any one of SEQ ID NOs: 6, 8, 43-44, 50, 192, 195-198, 201-213, 216-220, 222, 266. The immunoglobulin heavy chain fusion may comprise an amino acid sequence comprising 50 or more amino acids based on or derived from any one of SEQ ID NOs: 6, 8, 43-44, 50, 192, 195-198, 201-213, 216-220, 222, 266. The immunoglobulin heavy chain fusion may comprise an amino acid sequence comprising 100 or more amino acids based on or derived from any one of SEQ ID NOs: 6, 8, 43-44, 50, 192, 195-198, 201-213, 216-220, 222, 266. The immunoglobulin heavy chain fusion may comprise an amino acid sequence comprising 200 or more amino acids based on or derived from any one of SEQ ID NOs: 6, 8, 43-44, 50, 192, 195-198, 201-213, 216-220, 222, 266. The amino acids may be consecutive. Alternatively, or additionally, the amino acids are nonconsecutive. In some embodiments, the immunoglobulin heavy chain fusion may comprise amino acids derived from any one of SEQ ID NOs: 6, 8, 43-44, 50, 192, 195-198, 201-213, 216-220, 222, 266 and amino acids not derived from any one of SEQ ID NOs: 6, 8, 43-44, 50, 192, 195-198, 201-213, 216-220, 222, 266. In some embodiments, the immunoglobulin heavy chain fusion may comprise amino acids derived from one or more of SEQ ID NOs: 6,8 and amino acids not derived from any one of SEQ ID NOs: 6, 8, 43-44, 50, 192, 195-198, 201-213, 216-220, 222, 266. In some embodiments, the immunoglobulin heavy chain fusion comprises amino acids derived from 1, 2, 3, 4, or 5 of SEQ ID NOs: 6, 8, 43-44, 50, 192, 195-198, 201-213, 216-220, 222, 266.

The immunoglobulin heavy chain fusion may be encoded by a nucleotide sequence that is based on or derived from any one of SEQ ID NOs: 2, 4, 10, 11, 17, 161, 164-167, 170-182, 185-189, 191, 265. The immunoglobulin heavy chain fusion may be encoded by a nucleotide sequence that is at least about 50% homologous to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 60%, 65%, 70%, 75%, 80%, 85%, 90%, 95%, or 97% homologous to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 70% homologous to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 80% homologous to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 50% identical to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 60%, 65%, 70%, 75%, 80%, 85%, 90%, 95%, or 97% identical to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 70% identical to any one of SEQ ID NOs: 2, 4, 10, 11, 17, 161, 164-167, 170-182, 185-189, 191, 265. The immunoglobulin heavy chain fusion may be encoded by a nucleotide sequence that is at least about 80% identical to any one of SEQ ID NOs: 2, 4, 10, 11, 17, 161, 164-167, 170-182, 185-189, 191, 265. The immunoglobulin heavy chain fusion may be encoded by a nucleotide sequence that is 100% identical to any one of SEQ ID NOs: 2, 4, 10, 11, 17, 161, 164-167, 170-182, 185-189, 191, 265.

The immunoglobulin heavy chain fusion may be encoded by a nucleotide sequence comprising 10, 20, 30, 40, 50, 60, 70, 80, 90, 100 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 125, 150, 175, 200, 225, 250, 275, 300, 325, 350, 375, 400, 425, 450, 475, 450, 500 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 600, 650, 700, 750, 800, 850, 900, 950, 1000 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 1100, 1200, 1300, 1400, 1500 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 100 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 500 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 1000 or more nucleotides based on or derived from any one of SEQ ID NOs: 2, 4, 10, 11, 17, 161, 164-167, 170-182, 185-189, 191, 265. The immunoglobulin heavy chain fusion may be encoded by a nucleotide sequence comprising 1300 or more nucleotides based on or derived from any one of SEQ ID NOs: 2, 4, 10, 11, 17, 161, 164-167, 170-182, 185-189, 191, 265. The nucleotides may be consecutive. Alternatively, or additionally, the nucleotides are nonconsecutive. In some embodiments, the immunoglobulin heavy chain fusion is encoded by a nucleotide sequence comprising nucleotides derived from any one of SEQ ID NOs: 2,4 and nucleotides not derived from any one of SEQ ID NOs: 2, 4, 10, 11, 17, 161, 164-167, 170-182, 185-189, 191, 265. In some embodiments, the immunoglobulin heavy chain fusion is encoded by a nucleotide sequence comprising nucleotides derived from one or more of SEQ ID NOs: 2, 4, 10, 11, 17, 161, 164-167, 170-182, 185-189, 191, 265 and nucleotides not derived from any one of SEQ ID NOs: 2, 4, 10, 11, 17, 161, 164-167, 170-182, 185-189, 191, 265. In some embodiments, the immunoglobulin heavy chain fusion is encoded by a nucleotide sequence derived from 1, 2, 3, 4, or 5 of SEQ ID NOs: 2, 4, 10, 11, 17, 161, 164-167, 170-182, 185-189, 191, 265.

### Immunoglobulin Fusion Proteins

In one feature of the invention, provided herein are immunoglobulin fusion proteins comprising (a) an immunoglobulin light chain fusion, and (b) a second immunoglobulin region derived from an immunoglobulin heavy chain, wherein the immunoglobulin light chain fusion is connected to the second immunoglobulin region by one or more disulfide bonds or a connecting peptide. The immunoglobulin light chain fusion comprises a first therapeutic peptide connected to the amino-terminus of a first immunoglobulin region derived from an immunoglobulin light chain. In some embodiments, the second immunoglobulin region is attached to a non-immunoglobulin region, creating a second immunoglobulin fusion. The non-immunoglobulin region may comprise a second therapeutic peptide. The non-immunoglobulin region may comprise an extender peptide. The non-immunoglobulin region may comprise a linker peptide. The non-immunoglobulin region may comprise a proteolytic cleavage site. The second therapeutic peptide may comprise an internal linker. In some embodiments, the second therapeutic peptide is attached to the amino- or carboxyl-terminus of the second immunoglobulin region. In some embodiments, the second therapeutic peptide is attached to one or more internal amino acids of the second immunoglobulin region. In some embodiments, the second therapeutic peptide is attached to amino acids of a loop portion within the second immunoglobulin region. In some embodiments, the therapeutic peptide is attached to the second immunoglobulin region using one or more extender and/or linker peptides. The immunoglobulin light chain fusion may further comprise one or more additional therapeutic peptides.

In one feature of the invention, provided herein are immunoglobulin fusion proteins comprising (a) an immunoglobulin heavy chain fusion, and (b) a second immunoglobulin region derived from an immunoglobulin light chain, wherein the immunoglobulin heavy chain fusion is connected to the second immunoglobulin region by one or more disulfide bonds or a connecting peptide. The immunoglobulin heavy chain fusion comprises a first therapeutic peptide connected to the amino-terminus of a first immunoglobulin region derived from an immunoglobulin heavy chain. In some embodiments, the second immunoglobulin region is attached to a non-immunoglobulin region, creating a second immunoglobulin fusion. The non-immunoglobulin region may comprise a second therapeutic peptide. The non-immunoglobulin region may comprise an extender peptide. The non-immunoglobulin region may comprise a linker peptide. The non-immunoglobulin region may comprise a proteolytic cleavage site. The second therapeutic peptide may comprise an internal linker. In some embodiments, the second therapeutic peptide is attached to the amino- or carboxyl-terminus of the second immunoglobulin region. In some embodiments, the second therapeutic peptide is attached to one or more internal amino acids of the second immunoglobulin region. In some embodiments, the second therapeutic peptide is attached to amino acids of a loop portion within the second immunoglobulin region. In some embodiments, the therapeutic peptide is attached to the second immunoglobulin region using one or more extender and/or linker peptides. The immunoglobulin heavy chain fusion may further comprise one or more additional therapeutic peptides.

In one feature of the invention, provided herein are immunoglobulin fusion proteins comprising (a) an immunoglobulin light chain fusion, and (b) an immunoglobulin heavy chain fusion. The immunoglobulin light chain fusion comprises a first therapeutic peptide connected to the amino-terminus of a first immunoglobulin region derived from an immunoglobulin light chain. The immunoglobulin heavy chain fusion comprises a first therapeutic peptide connected to the amino-terminus of a first immunoglobulin region derived from an immunoglobulin heavy chain. In some embodiments, the immunoglobulin light chain fusion further comprises one or more additional therapeutic peptides. In some embodiments, the immunoglobulin heavy chain fusion comprises one or more additional therapeutic peptides.

In one feature of the invention, provided herein are immunoglobulin fusion proteins comprising (a) an immunoglobulin light chain fusion, and (b) a second immunoglobulin region, wherein the immunoglobulin light chain fusion comprises a first therapeutic peptide connected to the amino-terminus of a first immunoglobulin region derived from an immunoglobulin light chain. The second immunoglobulin region may be derived from an immunoglobulin heavy chain. The second immunoglobulin region may be derived from an immunoglobulin light chain. The second immunoglobulin region may be connected to one or more non-immunoglobulin regions, creating a second immunoglobulin fusion. The non-immunoglobulin region may comprise a second therapeutic peptide. The non-immunoglobulin region may comprise an extender peptide. The non-immunoglobulin region may comprise a linker peptide. The non-immunoglobulin region may comprise a proteolytic cleavage site. The second therapeutic peptide may comprise an internal linker. In some embodiments, the second therapeutic peptide is attached to the amino- or carboxyl-terminus of the second immunoglobulin region. In some embodiments, the second therapeutic peptide is attached to one or more internal amino acids of the second immunoglobulin region. In some embodiments, the second therapeutic peptide is attached to amino acids of a loop portion within the second immunoglobulin region. In some embodiments, the therapeutic peptide is attached to the second immunoglobulin region using one or more extender and/or linker peptides. The immunoglobulin light chain fusion may further comprise one or more additional therapeutic peptides.

In one feature of the invention, provided herein are immunoglobulin fusion proteins comprising (a) an immunoglobulin heavy chain fusion, and (b) a second immunoglobulin region, wherein the immunoglobulin heavy chain fusion comprises a first therapeutic peptide connected to the amino-terminus of a first immunoglobulin region derived from an immunoglobulin heavy chain. The second immunoglobulin region may be derived from an immunoglobulin heavy chain. The second immunoglobulin region may be derived from an immunoglobulin light chain. The second immunoglobulin region may be connected to one or more non-immunoglobulin regions, creating a second immunoglobulin fusion. The non-immunoglobulin region may comprise a second therapeutic peptide. The non-immunoglobulin region may comprise an extender peptide. The non-immunoglobulin region may comprise a linker peptide. The non-immunoglobulin region may comprise a proteolytic cleavage site. The second therapeutic peptide may comprise an internal linker. In some embodiments, the second therapeutic peptide is attached to the amino- or carboxyl-terminus of the second immunoglobulin region. In some embodiments, the second therapeutic peptide is attached to one or more internal amino acids of the second immunoglobulin region. In some embodiments, the second therapeutic peptide is attached to amino acids of a loop portion within the second immunoglobulin region. In some embodiments, the therapeutic peptide is attached to the second immunoglobulin region using one or more extender and/or linker peptides. The immunoglobulin heavy chain fusion may further comprise one or more additional therapeutic peptides.

The immunoglobulin fusion protein may comprise an immunoglobulin heavy chain fusion that is based on or derived from any one or more of SEQ ID NOs: 6, 8, 43, 44, 50, 192, 195-198, 201-213, 216-220, 222, 266.

The immunoglobulin fusion protein may comprise a second immunoglobulin region derived from an immunoglobulin heavy chain including any one or more of SEQ ID NOs: 6, 8, 43, 44, 50, 192, 195-198, 201-213, 216-220, 222, 266.

The immunoglobulin fusion protein may comprise an immunoglobulin light chain fusion that is based on or derived from any one or more of SEQ ID NOs: 5, 7, 42, 45-49, 51-74, 193, 194, 199, 200, 214, 215, 221.

The immunoglobulin fusion protein may comprise a second immunoglobulin region derived from an immunoglobulin light chain including any one or more of SEQ ID NOs: 5, 7, 42, 45-49, 51-74, 193, 194, 199, 200, 214, 215, 221.

The immunoglobulin fusion protein may comprise (a) a region of an immunoglobulin heavy chain that is based on or derived from any one or more of SEQ ID NOs: 6, 8, 43, 44, 50, 192, 195-198, 201-213, 216-220, 222, 266; and (b) a region of an immunoglobulin light chain that is based on or derived from any one or more of SEQ ID NOs: 5, 7, 42, 45-49, 51-74, 193, 194, 199, 200, 214, 215, 221. The immunoglobulin fusion protein may comprise (a) a region of an immunoglobulin heavy chain comprising an amino acid sequence that is at least about 50% identical to SEQ ID NOs 6, 8, 43, 44, 50, 192, 195-198, 201-213, 216-220, 222, 266; and (b) a region of an immunoglobulin light chain comprising an amino acid sequence that is at least about 50% identical to SEQ ID NOs: 5, 7, 42, 45-49, 51-74, 193, 194, 199, 200, 214, 215, 221. The region of an immunoglobulin heavy chain may comprise an amino acid sequence that is at least about 60%, 70%, 75%, 80%, 90%, 95%, or 97% identical to SEQ ID NOs: 6, 8, 43, 44, 50, 192, 195-198, 201-213, 216-220, 222, 266. The region of an immunoglobulin heavy chain may comprise an amino acid sequence that is 100% identical to SEQ ID NOs: 6, 8, 43, 44, 50, 192, 195-198, 201-213, 216-220, 222, 266. The region of an immunoglobulin light chain may comprise an amino acid sequence that is at least about 60%, 70%, 75%, 80%, 90%, 95%, or 97% identical to SEQ ID NOs: 5, 7, 42, 45-49, 51-74, 193, 194, 199, 200, 214, 215, 221. The region of an immunoglobulin light chain may comprise an amino acid sequence that is 100% identical to SEQ ID NOs: 5, 7, 42, 45-49, 51-74, 193, 194, 199, 200, 214, 215, 221.

The immunoglobulin fusion protein may comprise (a) a region of an immunoglobulin heavy chain encoded by a nucleotide sequence of SEQ ID NOs: 2, 4, 10, 11, 17, 161, 164-167, 170-182, 185-189, 191, 265; and (b) a region of an immunoglobulin light chain encoded by a nucleotide sequence of SEQ ID NOs: 1, 3, 9, 12-16, 18-41, 162, 163, 168, 169, 183, 184, 190. The immunoglobulin protein may comprise (a) a region of an immunoglobulin heavy chain encoded by a nucleotide sequence that is at least 50% or more identical to a nucleotide sequence of SEQ ID NOs: 2, 4, 10, 11, 17, 161, 164-167, 170-182, 185-189, 191, 265; and (b) a region of an immunoglobulin light chain encoded by a nucleotide sequence that is at least 50% or more identical to a nucleotide sequence of SEQ ID NOs: 1, 3, 9, 12-16, 18-41, 162, 163, 168, 169, 183, 184, 190. The region of an immunoglobulin heavy chain may be encoded by a nucleotide sequence that is at least 60%, 70%, 75%, 80%, 90%, 95%, or 97% or more identical to a nucleotide sequence of SEQ ID NOs: 2, 4, 10, 11, 17, 161, 164-167, 170-182, 185-189, 191, 265. The region of an immunoglobulin heavy chain may be encoded by a nucleotide sequence that is 100% identical to a nucleotide sequence of SEQ ID NOs: 2, 4, 10, 11, 17, 161, 164-167, 170-182, 185-189, 191, 265. The region of an immunoglobulin light chain may be encoded by a nucleotide sequence that is at least 60%, 70%, 75%, 80%, 90%, 95%, or 97% or more identical to a nucleotide sequence of SEQ ID NOs: 1, 3, 9, 12-16, 18-41, 162, 163, 168, 169, 183, 184, 190. The region of an immunoglobulin light chain may be encoded by a nucleotide sequence that is 100% identical to a nucleotide sequence of SEQ ID NOs: 1, 3, 9, 12-16, 18-41, 162, 163, 168, 169, 183, 184, 190.

In some embodiments, provided herein are immunoglobulin glucagon fusion proteins. In some embodiments, the immunoglobulin glucagon fusion proteins comprise an immunoglobulin light chain and/or heavy chain region fused at the amino terminus with a glucagon peptide, glucagon derived peptide such as ZP1, and/or a glucagon like peptide such as GLP-1 and/or GLP-2. In some embodiments, the immunoglobulin glucagon fusion proteins further comprise a second immunoglobulin light chain and/or heavy chain. In some embodiments, an immunoglobulin glucagon fusion protein refers to a first immunoglobulin chain comprising an amino-terminal glucagon peptide or derivative thereof and a second immunoglobulin chain. In some embodiments, the first immunoglobulin glucagon fusion protein is co-expressed with the second immunoglobulin chain. In some embodiments, the immunoglobulin glucagon fusion proteins are configured to treat a metabolic disease such as obesity and/or diabetes. In some embodiments, the immunoglobulin glucagon fusion proteins (including glucagon-like fusion proteins) are configured to treat short bowel syndrome. In some embodiments, the immunoglobulin glucagon fusion proteins (including glucagon-like fusion proteins) are configured to treat inflammatory bowel disease. The immunoglobulin glucagon fusion protein may comprise (a) a first immunoglobulin fusion protein comprising an amino acid sequence that is based on or derived from any one of SEQ ID NOs: 195, 196; and (b) a second immunoglobulin protein comprising an amino acid sequence that is based on or derived from SEQ ID NO: 7. The immunoglobulin glucagon fusion protein may comprise (a) a first immunoglobulin fusion protein comprising an amino acid sequence that is at least about 50% identical to any one of SEQ ID NOs: 195, 196; and (b) a second immunoglobulin protein comprising an amino acid sequence that is at least about 50% identical to SEQ ID NO: 7. The first immunoglobulin glucagon protein may comprise an amino acid sequence that is at least about 60%, 70%, 75%, 80%, 90%, 95%, or 97% identical to any one of SEQ ID NOs: 195, 196. The second immunoglobulin protein may comprise an amino acid sequence that is at least about 60%, 70%, 75%, 80%, 90%, 95%, or 97% identical to SEQ ID NO: 7.

The immunoglobulin glucagon fusion protein may comprise (a) a first immunoglobulin fusion protein encoded by a nucleotide sequence of any one of SEQ ID NOs: 164, 165; and (b) a second immunoglobulin protein encoded by a nucleotide sequence of SEQ ID NO: 3. The immunoglobulin glucagon fusion protein may comprise (a) a first immunoglobulin fusion protein encoded by a nucleotide sequence that is at least 50% or more homologous to a nucleotide sequence of any one of SEQ ID NOs: 164, 165; and (b) a second immunoglobulin protein encoded by a nucleotide sequence that is at least 50% or more homologous to a nucleotide sequence of SEQ ID NO: 3. In some embodiments, the first immunoglobulin fusion protein is encoded by a nucleotide sequence that is at least 60%, 70%, 75%, 80%, 90%, 95%, or 97% or more homologous to a nucleotide sequence of any one of SEQ ID NOs: 164, 165. In some embodiments, the second immunoglobulin protein is encoded by a nucleotide sequence that is at least 60%, 70%, 75%, 80%, 90%, 95%, or 97% or more homologous to a nucleotide sequence of SEQ ID NO: 3.

The immunoglobulin glucagon fusion protein may comprise (a) a first immunoglobulin fusion protein comprising an amino acid sequence that is based on or derived from any one of SEQ ID NOs: 199, 200; and (b) a second immunoglobulin protein comprising an amino acid sequence that is based on or derived from SEQ ID NO: 8. The immunoglobulin glucagon fusion protein may comprise (a) a first immunoglobulin fusion protein comprising an amino acid sequence that is at least about 50% identical to any one of SEQ ID NOs: 199, 200; and (b) a second immunoglobulin protein comprising an amino acid sequence that is at least about 50% identical to SEQ ID NO: 8. The first immunoglobulin glucagon protein may comprise an amino acid sequence that is at least about 60%, 70%, 75%, 80%, 90%, 95%, or 97% identical to any one of SEQ ID NOs: 199, 200. The second immunoglobulin protein may comprise an amino acid sequence that is at least about 60%, 70%, 75%, 80%, 90%, 95%, or 97% identical to SEQ ID NO: 8.

The immunoglobulin glucagon fusion protein may comprise (a) a first immunoglobulin fusion protein encoded by a nucleotide sequence of any one of SEQ ID NOs: 168, 169; and (b) a second immunoglobulin protein encoded by a nucleotide sequence of SEQ ID NO: 4. The immunoglobulin glucagon fusion protein may comprise (a) a first immunoglobulin fusion protein encoded by a nucleotide sequence that is at least 50% or more homologous to a nucleotide sequence of any one of SEQ ID NOs: 168, 169; and (b) a second immunoglobulin protein encoded by a nucleotide sequence that is at least 50% or more homologous to a nucleotide sequence of SEQ ID NO: 4. In some embodiments, the first immunoglobulin fusion protein is encoded by a nucleotide sequence that is at least 60%, 70%, 75%, 80%, 90%, 95%, or 97% or more homologous to a nucleotide sequence of any one of SEQ ID NOs: 168, 169. In some embodiments, the second immunoglobulin protein is encoded by a nucleotide sequence that is at least 60%, 70%, 75%, 80%, 90%, 95%, or 97% or more homologous to a nucleotide sequence of SEQ ID NO: 4.

The immunoglobulin glucagon fusion protein may comprise (a) a first immunoglobulin fusion protein comprising an amino acid sequence that is based on or derived from any one of SEQ ID NOs: 218-220; and (b) a second immunoglobulin protein comprising an amino acid sequence that is based on or derived from SEQ ID NO: 7. The immunoglobulin glucagon fusion protein may comprise (a) a first immunoglobulin fusion protein comprising an amino acid sequence that is at least about 50% identical to any one of SEQ ID NOs: 218-220; and (b) a second immunoglobulin protein comprising an amino acid sequence that is at least about 50% identical to SEQ ID NO: 7. The first immunoglobulin glucagon protein may comprise an amino acid sequence that is at least about 60%, 70%, 75%, 80%, 90%, 95%, or 97% identical to any one of SEQ ID NOs: 218-220. The second immunoglobulin protein may comprise an amino acid sequence that is at least about 60%, 70%, 75%, 80%, 90%, 95%, or 97% identical to SEQ ID NO: 7.

The immunoglobulin glucagon fusion protein may comprise (a) a first immunoglobulin fusion protein encoded by a nucleotide sequence of any one of SEQ ID NOs: 187-189; and (b) a second immunoglobulin protein encoded by a nucleotide sequence of SEQ ID NO: 3. The immunoglobulin glucagon fusion protein may comprise (a) a first immunoglobulin fusion protein encoded by a nucleotide sequence that is at least 50% or more homologous to a nucleotide sequence of any one of SEQ ID NOs: 187-189; and (b) a second immunoglobulin protein encoded by a nucleotide sequence that is at least 50% or more homologous to a nucleotide sequence of SEQ ID NO: 3. In some embodiments, the first immunoglobulin fusion protein is encoded by a nucleotide sequence that is at least 60%, 70%, 75%, 80%, 90%, 95%, or 97% or more homologous to a nucleotide sequence of any one of SEQ ID NOs: 187-189. In some embodiments, the second immunoglobulin protein is encoded by a nucleotide sequence that is at least 60%, 70%, 75%, 80%, 90%, 95%, or 97% or more homologous to a nucleotide sequence of SEQ ID NO: 3.

The immunoglobulin glucagon fusion protein may comprise (a) a first immunoglobulin fusion protein comprising an amino acid sequence that is based on or derived from SEQ ID NO: 221; and (b) a second immunoglobulin protein comprising an amino acid sequence that is based on or derived from SEQ ID NO: 8. The immunoglobulin glucagon fusion protein may comprise (a) a first immunoglobulin fusion protein comprising an amino acid sequence that is at least about 50% identical to SEQ ID NO: 221; and (b) a second immunoglobulin protein comprising an amino acid sequence that is at least about 50% identical to SEQ ID NO: 8. The first immunoglobulin glucagon protein may comprise an amino acid sequence that is at least about 60%, 70%, 75%, 80%, 90%, 95%, or 97% identical to SEQ ID NO: 221. The second immunoglobulin protein may comprise an amino acid sequence that is at least about 60%, 70%, 75%, 80%, 90%, 95%, or 97% identical to SEQ ID NO: 8.

The immunoglobulin glucagon fusion protein may comprise (a) a first immunoglobulin fusion protein encoded by a nucleotide sequence of SEQ ID NO: 190; and (b) a second immunoglobulin protein encoded by a nucleotide sequence of SEQ ID NO: 4. The immunoglobulin glucagon fusion protein may comprise (a) a first immunoglobulin fusion protein encoded by a nucleotide sequence that is at least 50% or more homologous to a nucleotide sequence of SEQ ID NO: 190; and (b) a second immunoglobulin protein encoded by a nucleotide sequence that is at least 50% or more homologous to a nucleotide sequence of SEQ ID NO: 4. In some embodiments, the first immunoglobulin fusion protein is encoded by a nucleotide sequence that is at least 60%, 70%, 75%, 80%, 90%, 95%, or 97% or more homologous to a nucleotide sequence of SEQ ID NO: 190. In some embodiments, the second immunoglobulin protein is encoded by a nucleotide sequence that is at least 60%, 70%, 75%, 80%, 90%, 95%, or 97% or more homologous to a nucleotide sequence of SEQ ID NO: 4.

In some embodiments, provided herein are immunoglobulin relaxin fusion proteins. In some embodiments, the immunoglobulin relaxin fusion proteins comprise an immunoglobulin light chain and/or heavy chain region fused at the amino terminus with a relaxin or a peptide derived from relaxin, which includes relaxins having internal linkers. In some embodiments, the immunoglobulin relaxin fusion proteins further comprise a second immunoglobulin light chain and/or heavy chain. In some embodiments, an immunoglobulin relaxin fusion protein refers to a first immunoglobulin chain comprising an amino-terminal relaxin peptide or derivative thereof and a second immunoglobulin chain. In some embodiments, the first immunoglobulin relaxin fusion protein is co-expressed with the second immunoglobulin chain. In some embodiments, the immunoglobulin relaxin fusion proteins are configured to treat a disease or condition of the heart. In some embodiments, the immunoglobulin relaxin fusion proteins treat a disease or condition including heart failure, acute coronary syndrome, atrial fibrillation, cardiac fibrosis, coronary artery disease, ischemia reperfusion associated with solid organ transplant (e.g., lung, kidney, liver, heart), cardiopulmonary bypass for organ protection (e.g., renal), ischemic stroke, corneal healing (ocular administration), diabetic nephropathy, cirrhosis, portal hypertension, diabetic would healing, systemic sclerosis, cervical ripening at time of labor, preeclampsia, portal hypertension, fibrosis, and combinations thereof. The immunoglobulin relaxin fusion protein may comprise (a) a first immunoglobulin fusion protein comprising an amino acid sequence that is based on or derived from any one of SEQ ID NOs: 201-213; and (b) a second immunoglobulin protein comprising an amino acid sequence that is based on or derived from SEQ ID NO: 7. The immunoglobulin relaxin fusion protein may comprise (a) a first immunoglobulin fusion protein comprising an amino acid sequence that is at least about 50% identical to any one of SEQ ID NOs: 201-213; and (b) a second immunoglobulin protein comprising an amino acid sequence that is at least about 50% identical to SEQ ID NO: 7. The first immunoglobulin relaxin protein may comprise an amino acid sequence that is at least about 60%, 70%, 75%, 80%, 90%, 95%, or 97% identical to any one of SEQ ID NOs: 201-213. The second immunoglobulin protein may comprise an amino acid sequence that is at least about 60%, 70%, 75%, 80%, 90%, 95%, or 97% identical to SEQ ID NO: 7.

The immunoglobulin relaxin fusion protein may comprise (a) a first immunoglobulin fusion protein encoded by a nucleotide sequence of any one of SEQ ID NOs: 170-182; and (b) a second immunoglobulin protein encoded by a nucleotide sequence of SEQ ID NO: 3. The immunoglobulin relaxin fusion protein may comprise (a) a first immunoglobulin fusion protein encoded by a nucleotide sequence that is at least 50% or more homologous to a nucleotide sequence of any one of SEQ ID NOs: 170-182; and (b) a second immunoglobulin protein encoded by a nucleotide sequence that is at least 50% or more homologous to a nucleotide sequence of SEQ ID NO: 3. In some embodiments, the first immunoglobulin fusion protein is encoded by a nucleotide sequence that is at least 60%, 70%, 75%, 80%, 90%, 95%, or 97% or more homologous to a nucleotide sequence of any one of SEQ ID NOs: 170-182. In some embodiments, the second immunoglobulin protein is encoded by a nucleotide sequence that is at least 60%, 70%, 75%, 80%, 90%, 95%, or 97% or more homologous to a nucleotide sequence of SEQ ID NO: 3.

The immunoglobulin relaxin fusion protein may comprise (a) a first immunoglobulin fusion protein comprising an amino acid sequence that is based on or derived from any one of SEQ ID NOs: 214, 215; and (b) a second immunoglobulin protein comprising an amino acid sequence that is based on or derived from SEQ ID NO: 8. The immunoglobulin relaxin fusion protein may comprise (a) a first immunoglobulin fusion protein comprising an amino acid sequence that is at least about 50% identical to any one of SEQ ID NOs: 214, 215; and (b) a second immunoglobulin protein comprising an amino acid sequence that is at least about 50% identical to SEQ ID NO: 8. The first immunoglobulin relaxin protein may comprise an amino acid sequence that is at least about 60%, 70%, 75%, 80%, 90%, 95%, or 97% identical to any one of SEQ ID NOs: 214, 215. The second immunoglobulin protein may comprise an amino acid sequence that is at least about 60%, 70%, 75%, 80%, 90%, 95%, or 97% identical to SEQ ID NO: 8.

The immunoglobulin relaxin fusion protein may comprise (a) a first immunoglobulin fusion protein encoded by a nucleotide sequence of any one of SEQ ID NOs: 183, 184; and (b) a second immunoglobulin protein encoded by a nucleotide sequence of SEQ ID NO: 4. The immunoglobulin relaxin fusion protein may comprise (a) a first immunoglobulin fusion protein encoded by a nucleotide sequence that is at least 50% or more homologous to a nucleotide sequence of any one of SEQ ID NOs: 183, 184; and (b) a second immunoglobulin protein encoded by a nucleotide sequence that is at least 50% or more homologous to a nucleotide sequence of SEQ ID NO: 4. In some embodiments, the first immunoglobulin fusion protein is encoded by a nucleotide sequence that is at least 60%, 70%, 75%, 80%, 90%, 95%, or 97% or more homologous to a nucleotide sequence of any one of SEQ ID NOs: 183, 184. In some embodiments, the second immunoglobulin protein is encoded by a nucleotide sequence that is at least 60%, 70%, 75%, 80%, 90%, 95%, or 97% or more homologous to a nucleotide sequence of SEQ ID NO: 4.

### Immunoglobulin Dual Fusion Proteins

Further disclosed herein are immunoglobulin dual fusion proteins comprising (a) a first immunoglobulin region attached to a first therapeutic peptide; and (b) a second therapeutic peptide, wherein the first therapeutic peptide is attached to the amino-terminus of the first immunoglobulin region. The first therapeutic peptide and the second therapeutic peptide may be the same. The first therapeutic peptide and the second therapeutic peptide may be different. The immunoglobulin dual fusion protein may further comprise a second immunoglobulin region. The second therapeutic peptide may be connected to the first immunoglobulin region or to a second immunoglobulin region. The first immunoglobulin region may comprise amino acids based on or derived from a light chain or a heavy chain of an immunoglobulin. The second immunoglobulin region may comprise amino acids based on or derived from a light chain or a heavy chain of an immunoglobulin. The first immunoglobulin region may comprise a light chain and the second immunoglobulin may comprise a heavy chain. The first immunoglobulin region may comprise a heavy chain and the second immunoglobulin may comprise a heavy chain. The second therapeutic peptide may be connected to any amino acid of the first or second immunoglobulin region, including, but not limited to, the amino terminus, carboxyl terminus, CDR, or loop of the immunoglobulin region. In some embodiments, the first immunoglobulin region and the second immunoglobulin region are connected via one or more disulfide bonds. In some embodiments, the first immunoglobulin region and the second immunoglobulin region are connected via a connecting peptide. The second therapeutic peptide may be attached to the first or second immunoglobulin region using extender and/or linker peptides. The second therapeutic peptide may be attached to the first or second immunoglobulin region using protease cleavage sites.

The dual fusion protein may comprise leptin and exendin-4 as the therapeutic peptides. The dual fusion protein may comprise leptin and a glucagon analog as the therapeutic peptides.

The dual fusion protein may comprise a heavy chain fusion based on or derived from an amino acid sequence that is at least about 50% homologous to SEQ ID NOs: 43, 44, 50. The dual fusion protein may comprise a heavy chain fusion based on or derived from an amino acid sequence that is at least about 55%, 60%, 65%, 70%, 75%, 80%, 85%, 90%, 95% or 97% homologous to SEQ ID NOs: 43, 44, 50. The dual fusion protein may comprise a heavy chain fusion based on or derived from an amino acid sequence that is at least about 70% homologous to SEQ ID NOs: 43, 44, 50. The dual fusion protein may comprise a heavy chain fusion based on or derived from an amino acid sequence that is at least about 80% homologous to SEQ ID NOs: 43, 44, 50. The dual fusion protein may comprise a heavy chain fusion based on or derived from an amino acid sequence that is at least about 90% homologous to SEQ ID NOs: 43, 44, 50. The dual fusion protein may comprise a light chain fusion based on or derived from an amino acid sequence that is at least about 50% homologous to SEQ ID NOs: 42, 45-49, 51-74, 193, 194, 199, 200, 214, 215, 221. The dual fusion protein may comprise a light chain fusion based on or derived from an amino acid sequence that is at least about 55%, 60%, 65%, 70%, 75%, 80%, 85%, 90%, 95% or 97% homologous to SEQ ID NOs: 42, 45-49, 51-74, 193, 194, 199, 200, 214, 215, 221. The dual fusion protein may comprise a light chain fusion based on or derived from an amino acid sequence that is at least about 70% homologous to SEQ ID NOs: 42, 45-49, 51-74, 193, 194, 199, 200, 214, 215, 221. The dual fusion protein may comprise a light chain fusion based on or derived from an amino acid sequence that is at least about 80% homologous to SEQ ID NOs: 42, 45-49, 51-74, 193, 194, 199, 200, 214, 215, 221. The dual fusion protein may comprise a light chain fusion based on or derived from an amino acid sequence that is at least about 90% homologous to SEQ ID NOs: 42, 45-49, 51-74, 193, 194, 199, 200, 214, 215, 221.

At least a portion of the dual fusion protein may be encoded by one or more nucleic acid sequences that are at least about 50% homologous to any one of SEQ ID NOs: 9-41, 161-191, 265. At least a portion of the dual fusion protein may be encoded by one or more nucleic acid sequences that are at least about 55%, 60%, 65%, 70%, 75%, 80%, 85%, 90%, 95% or 97% homologous to any one of SEQ ID NOs: 9-41, 161-191, 265. At least a portion of the dual fusion protein may be encoded by one or more nucleic acid sequences that are at least about 70% homologous to any one of SEQ ID NOs: 9-41, 161-191, 265. At least a portion of the dual fusion protein may be encoded by one or more nucleic acid sequences that are at least about 80% homologous to any one of SEQ ID NOs: 9-41, 161-191, 265. At least a portion of the dual fusion protein may be encoded by one or more nucleic acid sequences that are at least about 90% homologous to any one of SEQ ID NOs: 9-41, 161-191, 265.

The dual fusion protein may comprise two or more therapeutic peptides, wherein at least one of the therapeutic peptides are based on or derived from an amino acid sequence that is at least about 50% homologous to any one of SEQ ID NOs: 95-114, 230-236. The therapeutic peptide may comprise an amino acid sequence that is at least about 55%, 60%, 65%, 70%, 75%, 80%, 85%, 90%, 95% or 97% homologous to any one of SEQ ID NOs: 95-114, 230-236. The therapeutic peptide may comprise an amino acid sequence that is at least about 70% homologous to any one of SEQ ID NOs: 95-114, 230-236. The therapeutic peptide may comprise an amino acid sequence that is at least about 80% homologous to any one of SEQ ID NOs: 95-114, 230-236. The therapeutic peptide may comprise an amino acid sequence that is at least about 90% homologous to any one of SEQ ID NOs: 95-114, 230-236.

In some embodiments, the dual fusion protein may comprise two or more therapeutic peptides, wherein at least one of the therapeutic peptides are encoded by a nucleotide sequence that is at least about 50% homologous to any one of SEQ ID NOs: 75-94, 223-229. The therapeutic peptide may be encoded by a nucleotide sequence that is at least about 55%, 60%, 65%, 70%, 75%, 80%, 85%, 90%, 95% or 97% homologous to any one of SEQ ID NOs:. The therapeutic peptide may be encoded by a nucleotide sequence that is at least about 70% homologous to any one of SEQ ID NOs: 75-94, 223-229. The therapeutic peptide may be encoded by a nucleotide sequence that is at least about 80% homologous to any one of SEQ ID NOs: 75-94, 223-229. The therapeutic peptide may be encoded by a nucleotide sequence that is at least about 90% homologous to any one of SEQ ID NOs: 75-94, 223-229.

The dual fusion protein may be comprise an immunoglobulin region that is based on or derived from an amino acid sequence that is at least about 50% homologous to any one of SEQ ID NOs: 5-8. The dual fusion protein may be comprise an immunoglobulin region that is based on or derived from an amino acid sequence that is at least about 55%, 60%, 65%, 70%, 75%, 80%, 85%, 90%, 95% or 97% homologous to any one of SEQ ID NOs: 5-8. The dual fusion protein may be comprise an immunoglobulin region that is based on or derived from an amino acid sequence that is at least about 70% homologous to any one of SEQ ID NOs: 5-8. The dual fusion protein may be comprise an immunoglobulin region that is based on or derived from an amino acid sequence that is at least about 80% homologous to any one of SEQ ID NOs: 5-8. The dual fusion protein may be comprise an immunoglobulin region that is based on or derived from an amino acid sequence that is at least about 90% homologous to any one of SEQ ID NOs: 5-8. The dual fusion protein may be comprise an immunoglobulin Fab region that is based on or derived from an amino acid sequence that is at least about 70%, 80%, 90% or 95% homologous to any one of SEQ ID NOs: 5-8.

The dual fusion protein may be comprise an immunoglobulin region that is encoded by one or more nucleotide sequences that are at least about 50% homologous to any one of SEQ ID NOs: 1-4. The dual fusion protein may be comprise an immunoglobulin region that is encoded by one or more nucleotide sequences that are at least about 55%, 60%, 65%, 70%, 75%, 80%, 85%, 90%, 95% or 97% homologous to any one of SEQ ID NOs: 1-4. The dual fusion protein may be comprise an immunoglobulin region that is encoded by one or more nucleotide sequences that are at least about 70% homologous to any one of SEQ ID NOs: 1-4. The dual fusion protein may be comprise an immunoglobulin region that is encoded by one or more nucleotide sequences that are at least about 80% homologous to any one of SEQ ID NOs: 1-4. The dual fusion protein may be comprise an immunoglobulin region that is encoded by one or more nucleotide sequences that are at least about 90% homologous to any one of SEQ ID NOs: 1-4. The dual fusion protein may be comprise an immunoglobulin Fab region that is encoded by one or more nucleotide sequences that are at least about 70%, 80%, 90% or 95% homologous to any one of SEQ ID NOs: 1-4.

Further disclosed herein are immunoglobulin Leptin/Exendin-4 dual fusion proteins. The immunoglobulin dual fusion protein may comprise (a) a first immunoglobulin fusion protein comprising an amino acid sequence that is based on or derived from SEQ ID NO: 42; and (b) a second immunoglobulin fusion protein comprising an amino acid sequence that is based on or derived from SEQ ID NOs: 43-44. The immunoglobulin dual fusion protein may comprise (a) a first immunoglobulin fusion protein comprising an amino acid sequence that is at least about 50% identical to SEQ ID NO: 42; and (b) a second immunoglobulin fusion protein comprising an amino acid sequence that is at least about 50% identical to SEQ ID NOs: 43-44. The first immunoglobulin fusion protein may comprise an amino acid sequence that is at least about 60%, 70%, 75%, 80%, 90%, 95%, or 97% identical to SEQ ID NO: 42. The second immunoglobulin fusion protein may comprise an amino acid sequence that is at least about 60%, 70%, 75%, 80%, 90%, 95%, or 97% identical to SEQ ID NOs: 43-44.

The immunoglobulin dual fusion protein may comprise (a) a first immunoglobulin fusion protein encoded by a nucleotide sequence of SEQ ID NO: 9; and (b) a second immunoglobulin fusion protein encoded by a nucleotide sequence of SEQ ID NOs: 10-11. The immunoglobulin dual fusion protein may comprise (a) a first immunoglobulin fusion protein encoded by a nucleotide sequence that is at least 50% or more homologous to a nucleotide sequence of SEQ ID NO: 9; and (b) a second immunoglobulin fusion protein encoded by a nucleotide sequence that is at least 50% or more homologous to a nucleotide sequence of SEQ ID NOs: 10-11. The first immunoglobulin fusion protein may be encoded by a nucleotide sequence that is at least 60%, 70%, 75%, 80%, 90%, 95%, or 97% or more homologous to a nucleotide sequence of SEQ ID NO: 9. The second immunoglobulin fusion protein may be encoded by a nucleotide sequence that is at least 60%, 70%, 75%, 80%, 90%, 95%, or 97% or more homologous to a nucleotide sequence of SEQ ID NOs: 10-11.

Further disclosed herein are immunoglobulin Leptin/ZP1CEX dual fusion proteins. The immunoglobulin dual fusion protein may comprise (a) a first immunoglobulin fusion protein comprising an amino acid sequence that is based on or derived from SEQ ID NO: 46; and (b) a second immunoglobulin fusion protein comprising an amino acid sequence that is based on or derived from SEQ ID NOs: 43-44. The immunoglobulin dual fusion protein may comprise (a) a first immunoglobulin fusion protein comprising an amino acid sequence that is at least about 50% identical to SEQ ID NO: 46; and (b) a second immunoglobulin fusion protein comprising an amino acid sequence that is at least about 50% identical to SEQ ID NOs: 43-44. The first immunoglobulin fusion protein may comprise an amino acid sequence that is at least about 60%, 70%, 75%, 80%, 90%, 95%, or 97% identical to SEQ ID NO: 46. The second immunoglobulin fusion protein may comprise an amino acid sequence that is at least about 60%, 70%, 75%, 80%, 90%, 95%, or 97% identical to SEQ ID NOs: 43-44.

The immunoglobulin dual fusion protein may comprise (a) a first immunoglobulin fusion protein encoded by a nucleotide sequence of SEQ ID NO: 13; and (b) a second immunoglobulin fusion protein encoded by a nucleotide sequence of SEQ ID NOs: 10-11. The immunoglobulin dual fusion protein may comprise (a) a first immunoglobulin fusion protein encoded by a nucleotide sequence that is at least 50% or more homologous to a nucleotide sequence of SEQ ID NO: 13; and (b) a second immunoglobulin fusion protein encoded by a nucleotide sequence that is at least 50% or more homologous to a nucleotide sequence of SEQ ID NOs: 10-11. The first immunoglobulin fusion protein may be encoded by a nucleotide sequence that is at least 60%, 70%, 75%, 80%, 90%, 95%, or 97% or more homologous to a nucleotide sequence of SEQ ID NO: 13. The second immunoglobulin fusion protein may be encoded by a nucleotide sequence that is at least 60%, 70%, 75%, 80%, 90%, 95%, or 97% or more homologous to a nucleotide sequence of SEQ ID NOs: 10-11.

Further disclosed herein are immunoglobulin exendin-4/glucagon dual fusion proteins. In some embodiments, the immunoglobulin exendin-4/glucagon dual fusion proteins are configured to treat a metabolic disease such as obesity and/or diabetes. The immunoglobulin dual fusion protein may comprise (a) a first immunoglobulin fusion protein comprising an amino acid sequence that is based on or derived from SEQ ID NO: 192; and (b) a second immunoglobulin fusion protein comprising an amino acid sequence that is based on or derived from any of SEQ ID NOs: 193-194. The immunoglobulin dual fusion protein may comprise (a) a first immunoglobulin fusion protein comprising an amino acid sequence that is at least about 50% identical to SEQ ID NO: 192; and (b) a second immunoglobulin fusion protein comprising an amino acid sequence that is at least about 50% identical to any of SEQ ID NOs: 193-194. The first immunoglobulin fusion protein may comprise an amino acid sequence that is at least about 60%, 70%, 75%, 80%, 90%, 95%, or 97% identical to SEQ ID NO: 192. The second immunoglobulin fusion protein may comprise an amino acid sequence that is at least about 60%, 70%, 75%, 80%, 90%, 95%, or 97% identical to any of SEQ ID NOs: 193-194.

The immunoglobulin dual fusion protein may comprise (a) a first immunoglobulin fusion protein encoded by a nucleotide sequence of SEQ ID NO: 161; and (b) a second immunoglobulin fusion protein encoded by a nucleotide sequence of SEQ ID NOs: 162-163. The immunoglobulin dual fusion protein may comprise (a) a first immunoglobulin fusion protein encoded by a nucleotide sequence that is at least 50% or more homologous to a nucleotide sequence of SEQ ID NO: 161; and (b) a second immunoglobulin fusion protein encoded by a nucleotide sequence that is at least 50% or more homologous to a nucleotide sequence of SEQ ID NOs: 162-163. The first immunoglobulin fusion protein may be encoded by a nucleotide sequence that is at least 60%, 70%, 75%, 80%, 90%, 95%, or 97% or more homologous to a nucleotide sequence of SEQ ID NO: 161. The second immunoglobulin fusion protein may be encoded by a nucleotide sequence that is at least 60%, 70%, 75%, 80%, 90%, 95%, or 97% or more homologous to a nucleotide sequence of SEQ ID NOs: 162-163.

Further disclosed herein are immunoglobulin exendin-4/ZP1 dual fusion proteins. In some embodiments, the immunoglobulin exendin-4/ZP1 dual fusion proteins are configured to treat a metabolic disease such as obesity and/or diabetes. The immunoglobulin dual fusion protein may comprise (a) a first immunoglobulin fusion protein comprising an amino acid sequence that is based on or derived from SEQ ID NO: 42; and (b) a second immunoglobulin fusion protein comprising an amino acid sequence that is based on or derived from any of SEQ ID NOs: 197-198. The immunoglobulin dual fusion protein may comprise (a) a first immunoglobulin fusion protein comprising an amino acid sequence that is at least about 50% identical to SEQ ID NO: 42; and (b) a second immunoglobulin fusion protein comprising an amino acid sequence that is at least about 50% identical to any of SEQ ID NOs: 197-198. The first immunoglobulin fusion protein may comprise an amino acid sequence that is at least about 60%, 70%, 75%, 80%, 90%, 95%, or 97% identical to SEQ ID NO: 42. The second immunoglobulin fusion protein may comprise an amino acid sequence that is at least about 60%, 70%, 75%, 80%, 90%, 95%, or 97% identical to any of SEQ ID NOs: 197-198.

The immunoglobulin dual fusion protein may comprise (a) a first immunoglobulin fusion protein encoded by a nucleotide sequence of SEQ ID NO: 9; and (b) a second immunoglobulin fusion protein encoded by a nucleotide sequence of SEQ ID NOs: 166-167. The immunoglobulin dual fusion protein may comprise (a) a first immunoglobulin fusion protein encoded by a nucleotide sequence that is at least 50% or more homologous to a nucleotide sequence of SEQ ID NO: 9; and (b) a second immunoglobulin fusion protein encoded by a nucleotide sequence that is at least 50% or more homologous to a nucleotide sequence of SEQ ID NOs: 166-167. The first immunoglobulin fusion protein may be encoded by a nucleotide sequence that is at least 60%, 70%, 75%, 80%, 90%, 95%, or 97% or more homologous to a nucleotide sequence of SEQ ID NO: 9. The second immunoglobulin fusion protein may be encoded by a nucleotide sequence that is at least 60%, 70%, 75%, 80%, 90%, 95%, or 97% or more homologous to a nucleotide sequence of SEQ ID NOs: 166-167.

Further disclosed herein are immunoglobulin exendin-4/glucagon-like (e.g., GLP-1, GLP-2) dual fusion proteins. In some embodiments, the immunoglobulin exendin-4/glucagon-like dual fusion proteins are configured to treat a metabolic disease such as obesity and/or diabetes. The immunoglobulin exendin-4/glucagon-like fusion protein may comprise (a) a first immunoglobulin fusion protein comprising an amino acid sequence that is based on or derived from any one of SEQ ID NOs: 216, 217; and (b) a second immunoglobulin fusion protein comprising an amino acid sequence that is based on or derived from SEQ ID NO: 42. The immunoglobulin exendin-4/glucagon-like fusion protein may comprise (a) a first immunoglobulin fusion protein comprising an amino acid sequence that is at least about 50% identical to any one of SEQ ID NOs: 216, 217; and (b) a second immunoglobulin fusion protein comprising an amino acid sequence that is at least about 50% identical to SEQ ID NO: 42. The first immunoglobulin fusion protein may comprise an amino acid sequence that is at least about 60%, 70%, 75%, 80%, 90%, 95%, or 97% identical to any one of SEQ ID NOs: 216, 217. The second immunoglobulin fusion protein may comprise an amino acid sequence that is at least about 60%, 70%, 75%, 80%, 90%, 95%, or 97% identical to SEQ ID NO: 42.

The immunoglobulin exendin-4/glucagon-like fusion protein may comprise (a) a first immunoglobulin fusion protein encoded by a nucleotide sequence of any one of SEQ ID NOs: 185, 186; and (b) a second immunoglobulin fusion protein encoded by a nucleotide sequence of SEQ ID NO: 9. The immunoglobulin exendin-4/glucagon-like fusion protein may comprise (a) a first immunoglobulin fusion protein encoded by a nucleotide sequence that is at least 50% or more homologous to a nucleotide sequence of any one of SEQ ID NOs: 185, 186; and (b) a second immunoglobulin fusion protein encoded by a nucleotide sequence that is at least 50% or more homologous to a nucleotide sequence of SEQ ID NO: 9. In some embodiments, the first immunoglobulin fusion protein is encoded by a nucleotide sequence that is at least 60%, 70%, 75%, 80%, 90%, 95%, or 97% or more homologous to a nucleotide sequence of any one of SEQ ID NOs: 185, 186. In some embodiments, the second immunoglobulin fusion protein is encoded by a nucleotide sequence that is at least 60%, 70%, 75%, 80%, 90%, 95%, or 97% or more homologous to a nucleotide sequence of SEQ ID NO: 9.

Second Immunoglobulin Fusions

In some embodiments, an immunoglobulin fusion protein comprises (a) a first therapeutic peptide attached to the amino-terminus of a first immunoglobulin region, and (b) a second immunoglobulin region. The second immunoglobulin region may be attached to one or more non-immunoglobulin regions to create a second immunoglobulin fusion. In some embodiments, a non-immunoglobulin region does not comprise an amino acid sequence that is greater than 80% identical to an amino acid sequence of an immunoglobulin. In some embodiments, a non-immunoglobulin region does not comprise an amino acid sequence greater than 50%, 55%, 60%, 65%, 70%, 75%, 80%, 85%, 90%, 95%, or 99% identical to an amino acid sequence of an immunoglobulin. In some embodiments, a peptide not derived from an immunoglobulin does not comprise an amino acid sequence 100% identical to an amino acid sequence of an immunoglobulin. In some embodiments, the non-immunoglobulin region comprises a therapeutic peptide and one or more extender peptides. In some embodiments, the non-immunoglobulin region comprises a therapeutic peptide and one or more linker peptides. In some embodiments, the immunoglobulin fusion protein comprises a protease cleavage site. In some embodiments, the non-immunoglobulin region comprises a protease cleavage site. In some embodiments, the therapeutic peptide comprises one or more internal linkers. In some embodiments, the non-immunoglobulin region is connected to the immunoglobulin region at a loop present in the immunoglobulin region. In some embodiments, the loop comprises amino acids of a complementarity determining region (CDR). The CDR may include CDR1, CDR2, CDR3, and CDR4. In some embodiments, the non-immunoglobulin region replaces at least a portion of an immunoglobulin region from which the immunoglobulin region is based on or derived from. The non-immunoglobulin region may replace at least a portion of a complementarity determining region. The non-immunoglobulin region may replace at least a portion of a variable domain. The non-immunoglobulin region may replace at least a portion of a constant domain. The non-immunoglobulin region may replace at least a portion of a heavy chain. The non-immunoglobulin region may replace at least a portion of a light chain.

Exemplary second immunoglobulin fusions are depicted by Formulas IA-XIIB.

Formula IA depicts a second immunoglobulin fusion comprising a second immunoglobulin region (A²) attached to a non-immunoglobulin region comprising an extender peptide (E¹) and a second therapeutic peptide (T²).

Formula IIA depicts a second immunoglobulin fusion comprising a second immunoglobulin region (A²) attached to a non-immunoglobulin region comprising two extender peptides (E¹ and E²) attached to a second therapeutic peptide (T²).

Formula IIIA depicts a second immunoglobulin fusion comprising a second immunoglobulin region (A²) attached to a non-immunoglobulin region comprising a linker (L¹) attached to a second therapeutic peptide (T²), with the linker and second therapeutic peptide located between two extender peptides (E¹ and E²).

Formula IVA depicts a second immunoglobulin fusion comprising a second immunoglobulin region (A²) attached to a non-immunoglobulin region comprising a proteolytic cleavage site (P¹) attached to a second therapeutic peptide (T²), with the proteolytic cleavage site and second therapeutic peptide located between two extender peptides (E¹ and E²). Formula IVB shows the clipped version of Formula VA, wherein the proteolytic cleavage site is cleaved by a protease, which results in release of one end of the second therapeutic peptide.

Formula VA depicts a second immunoglobulin fusion comprising a second immunoglobulin region (A²) attached to a non-immunoglobulin region comprising a second therapeutic peptide (T²) attached to a linker (L¹) and a proteolytic cleavage site (P¹), wherein the second therapeutic peptide, linker and proteolytic cleavage site are located between two extender peptides (E¹ and E²). Formula VB shows the clipped version of Formula VA, wherein the proteolytic cleavage site is cleaved by a protease, which results in release of one end of the second therapeutic peptide.

Formula VIA depicts a second immunoglobulin fusion comprising a second immunoglobulin region (A²) attached to a non-immunoglobulin region comprising two extender peptides (E¹ and E²), two linkers (L¹ and L²), two proteolytic cleavage sites (P¹ and P²) and a second therapeutic peptide (T²). Formula VIB shows the clipped version of Formula VIA, wherein the proteolytic cleavage sites located on the N- and C-termini of the second therapeutic peptide are cleaved by a protease, which results in release of the second therapeutic peptide from the second immunoglobulin fusion.

Formula VIIA depicts a second immunoglobulin fusion comprising a second immunoglobulin region (A²) attached to a non-immunoglobulin region comprising a second therapeutic peptide (T²).

Formula VIIIA depicts a second immunoglobulin fusion comprising a second immunoglobulin region (A²) attached to a non-immunoglobulin region comprising a linker (L¹) attached to a second therapeutic peptide (T²).

Formula IXA depicts a second immunoglobulin fusion comprising a second immunoglobulin region (A²) attached to a non-immunoglobulin region comprising a linker (L¹), a proteolytic cleavage site (P¹) and a second therapeutic peptide (T²), wherein the proteolytic cleavage site is located between the linker and the second therapeutic peptide.

Formula XA depicts a second immunoglobulin fusion protein comprising a second immunoglobulin region (A²) attached to a non-immunoglobulin region comprising a proteolytic cleavage site (P¹) attached to a second therapeutic peptide (T²). Formula XB shows the clipped version of Formula XA, wherein the proteolytic cleavage site is cleaved by a protease, which results in release of one end of the second therapeutic peptide.

Formula XIA depicts a second immunoglobulin fusion comprising a second immunoglobulin region (A²) attached to a non-immunoglobulin region comprising a linker (L¹), a second therapeutic peptide (T²), and a proteolytic cleavage site (P′), wherein the second therapeutic peptide is located between the linker and the proteolytic cleavage site. Formula XIB shows the clipped version of Formula XIA, wherein the proteolytic cleavage site is cleaved by a protease, which results in release of one end of the second therapeutic peptide.

Formula XIIA depicts a second immunoglobulin fusion comprising a second immunoglobulin region (A²) attached to a non-immunoglobulin region comprising two linkers (L¹ and L²), two proteolytic cleavage sites (P¹ and P²) and a second therapeutic peptide (T²). Formula XIIB shows the clipped version of Formula XIIA, wherein the proteolytic cleavage sites located on the N- and C-termini of the second therapeutic peptide are cleaved by a protease, which results in release of the second therapeutic peptide from the second immunoglobulin fusion.

### Immunoglobulin Region

The immunoglobulin fusion proteins disclosed herein comprise one or more immunoglobulin regions. The immunoglobulin region may comprise an immunoglobulin or a fragment thereof. The immunoglobulin region may comprise at least a portion of an immunoglobulin heavy chain, immunoglobulin light chain, or a combination thereof. The immunoglobulin region may comprise two or more immunoglobulin chains or portions thereof. The immunoglobulin region may comprise three or more immunoglobulin chains or portions thereof. The immunoglobulin region may comprise four or more immunoglobulin chains or portions thereof. The immunoglobulin region may comprise five or more immunoglobulin chains or portions thereof. The immunoglobulin region may comprise two immunoglobulin heavy chains and two immunoglobulin light chains.

The immunoglobulin region may comprise an entire immunoglobulin molecule or any polypeptide comprising fragment of an immunoglobulin including, but not limited to, heavy chain, light chain, variable domain, constant domain, complementarity determining region (CDR), framework region, fragment antigen binding (Fab) region, Fab′, F(ab′)2, F(ab′)3, Fab′, fragment crystallizable (Fc) region, single chain variable fragment (scFV), di-scFv, single domain immunoglobulin, trifunctional immunoglobulin, chemically linked F(ab′)2, and any combination thereof. In some embodiments, an immunoglobulin heavy chain may comprise an entire heavy chain or a portion of a heavy chain. For example, a variable domain or region thereof derived from a heavy chain may be referred to as a heavy chain or a region of a heavy chain. In some embodiments, an immunoglobulin light chain may comprise an entire light chain or a portion of a light chain. For example, a variable domain or region thereof derived from a light chain may be referred to as a light chain or a region of a light chain. A single domain immunoglobulin includes, but is not limited to, a single monomeric variable immunoglobulin domain, for example, a shark variable new antigen receptor immunoglobulin fragment (VNAR).

The immunoglobulin may be derived from any type known to one of skill in the art including, but not limited to, IgA, IgD, IgE, IgG, IgM, IgY, IgW. The immunoglobulin region may comprise one or more units, including but not limited to, 1, 2, 3, 4, and 5 units. Functional units may include, but are not limited to, non-immunoglobulin regions, heavy chain, light chain, variable domain, constant domain, complementarity determining region (CDR), framework region, fragment antigen binding (Fab) region, Fab′, F(ab′)2, F(ab′)3, Fab′, fragment crystallizable (Fc) region, single chain variable fragment (scFV), di-scFv, single domain immunoglobulin, trifunctional immunoglobulin, chemically linked F(ab′)2, and any combination or fragments thereof. Non-immunoglobulin regions include, but are not limited to, carbohydrates, lipids, small molecules and therapeutic peptides. The immunoglobulin region may comprise one or more units connected by one or more disulfide bonds. The immunoglobulin region may comprise one or more units connected by a peptide linker, for example, a scFv immunoglobulin. The immunoglobulin may be a recombinant immunoglobulin including immunoglobulins with amino acid mutations, substitutions, and/or deletions. The immunoglobulin may be a recombinant immunoglobulin comprising chemical modifications. The immunoglobulin may comprise a whole or part of an immunoglobulin-drug conjugate.

The immunoglobulin region may comprise at least a portion of an immunoglobulin heavy chain. The immunoglobulin region may comprise one or more immunoglobulin heavy chains or a portion thereof. The immunoglobulin region may comprise two or more immunoglobulin heavy chains or a portion thereof. The immunoglobulin region may comprise an amino acid sequence that is at least about 50% homologous to an immunoglobulin heavy chain. The immunoglobulin region may comprise an amino acid sequence that is at least about 60%, 65%, 70%, 75%, 80%, 85%, 90%, 92%, 95%, or 97% or more homologous to an immunoglobulin heavy chain. The immunoglobulin region may comprise an amino acid sequence that is at least about 70% homologous to an immunoglobulin heavy chain. The immunoglobulin region may comprise an amino acid sequence that is at least about 80% homologous to an immunoglobulin heavy chain. The immunoglobulin region may comprise an amino acid sequence that is at least about 90% homologous to an immunoglobulin heavy chain. The immunoglobulin heavy chain may comprise SEQ ID NOs: 6, 8. In some embodiments, the immunoglobulin region comprises an amino acid sequence that is at least about 50%, 55%, 60%, 65%, 70%, 75%, 80%, 85%, 90%, 95%, or 97% homologous to an amino acid sequence of any one of SEQ ID NOs: 6, 8. In some embodiments, the immunoglobulin region comprises an amino acid sequence that is at least about 50%, 55%, 60%, 65%, 70%, 75%, 80%, 85%, 90%, 95%, or 97% identical to an amino acid sequence of any one of SEQ ID NOs: 6, 8.

The immunoglobulin region may comprise an amino acid sequence comprising 5, 10, 15, 20, 25, 30, 35, 40, 45, 50, 60, 70, 80, 90 or more amino acids of an immunoglobulin heavy chain. The immunoglobulin region may comprise an amino acid sequence comprising 100, 150, 200, 250, 300, 350, 400, 450, 500, 600, 700, 800, 900 or more amino acids of an immunoglobulin heavy chain. The amino acids may be consecutive. Alternatively, or additionally, the amino acids are non-consecutive.

The immunoglobulin heavy chain may be encoded by a nucleotide sequence based on or derived from SEQ ID NOs: 2, 4. The immunoglobulin heavy chain may be encoded by a nucleotide sequence that is at least about 50% homologous to SEQ ID NOs: 2, 4. The immunoglobulin heavy chain may be encoded by a nucleotide sequence that is at least about 60%, 65%, 70%, 75%, 80%, 85%, 90%, 92%, 95%, or 97% or more homologous to SEQ ID NOs: 2, 4. The immunoglobulin heavy chain may be encoded by a nucleotide sequence that is at least about 75% homologous to SEQ ID NOs: 2, 4. The immunoglobulin heavy chain may be encoded by a nucleotide sequence that is at least about 85% homologous to SEQ ID NOs: 2, 4. In some embodiments, the immunoglobulin region is encoded by a nucleotide sequence that is at least about 50%, 55%, 60%, 65%, 70%, 75%, 80%, 85%, 90%, 95%, or 97% homologous to a nucleotide sequence of any one of SEQ ID NOs: 2, 4. In some embodiments, the immunoglobulin region is encoded by a nucleotide sequence that is at least about 50%, 55%, 60%, 65%, 70%, 75%, 80%, 85%, 90%, 95%, or 97% identical to a nucleotide sequence of any one of SEQ ID NOs: 2, 4.

The immunoglobulin region may comprise at least a portion of an immunoglobulin light chain. The immunoglobulin region may comprise one or more immunoglobulin light chains or a portion thereof. The immunoglobulin region may comprise two or more immunoglobulin light chains or a portion thereof. The immunoglobulin region may comprise an amino acid sequence that is at least about 50% homologous to an immunoglobulin light chain. The immunoglobulin region may comprise an amino acid sequence that is at least about 60%, 65%, 70%, 75%, 80%, 85%, 90%, 92%, 95%, or 97% or more homologous to an immunoglobulin light chain. The immunoglobulin region may comprise an amino acid sequence that is at least about 70% homologous to an immunoglobulin light chain. The immunoglobulin region may comprise an amino acid sequence that is at least about 80% homologous to an immunoglobulin light chain. The immunoglobulin region may comprise an amino acid sequence that is at least about 90% homologous to an immunoglobulin light chain. The immunoglobulin light chain may comprise SEQ ID NOs: 5, 7. In some embodiments, the immunoglobulin region comprises an amino acid sequence that is at least about 50%, 55%, 60%, 65%, 70%, 75%, 80%, 85%, 90%, 95%, or 97% homologous to an amino acid sequence of any one of SEQ ID NOs: 5, 7. In some embodiments, the immunoglobulin region comprises an amino acid sequence that is at least about 50%, 55%, 60%, 65%, 70%, 75%, 80%, 85%, 90%, 95%, or 97% identical to an amino acid sequence of any one of SEQ ID NOs: 5, 7.

The immunoglobulin region may comprise an amino acid sequence comprising 5, 10, 15, 20, 25, 30, 35, 40, 45, 50, 60, 70, 80, 90 or more amino acids of an immunoglobulin light chain. The immunoglobulin region may comprise an amino acid sequence comprising 100, 150, 200, 250, 300, 350, 400, 450, 500, 600, 700, 800, 900 or more amino acids of an immunoglobulin light chain. The amino acids may be consecutive. Alternatively, or additionally, the amino acids are non-consecutive.

The immunoglobulin light chain may be encoded by a nucleotide sequence based on or derived from SEQ ID NOs: 1, 3. The immunoglobulin light chain may be encoded by a nucleotide sequence that is at least about 50% homologous to SEQ ID NOs: 1, 3. The immunoglobulin light chain may be encoded by a nucleotide sequence that is at least about 60%, 65%, 70%, 75%, 80%, 85%, 90%, 92%, 95%, or 97% or more homologous to SEQ ID NOs: 1, 3. The immunoglobulin light chain may be encoded by a nucleotide sequence that is at least about 75% homologous to SEQ ID NOs: 1, 3. The immunoglobulin light chain may be encoded by a nucleotide sequence that is at least about 85% homologous to SEQ ID NOs: 1, 3. In some embodiments, the immunoglobulin region is encoded by a nucleotide sequence that is at least about 50%, 55%, 60%, 65%, 70%, 75%, 80%, 85%, 90%, 95%, or 97% homologous to a nucleotide sequence of any one of SEQ ID NOs: 1, 3. In some embodiments, the immunoglobulin region is encoded by a nucleotide sequence that is at least about 50%, 55%, 60%, 65%, 70%, 75%, 80%, 85%, 90%, 95%, or 97% identical to a nucleotide sequence of any one of SEQ ID NOs: 1, 3.

The immunoglobulin region may comprise at least a portion of a variable domain. The immunoglobulin region may comprise one or more variable domains or portions thereof. The immunoglobulin region may comprise 2, 3, 4, 5 or more variable domains or portions thereof. The immunoglobulin region may comprise an amino acid sequence comprising 10, 20, 30, 40, 50, 60, 70, 80, 90, 100, 120, 140, 160, 180, 200, 225, 250, 275, 300, 350, 400, 500 or more amino acids based on or derived from an amino acid sequence of one or more variable domains. The amino acids may be consecutive. The amino acids may be non-consecutive.

The immunoglobulin region may comprise at least a portion of a constant domain. The immunoglobulin region may comprise one or more constant domains or portions thereof. The immunoglobulin region may comprise 2, 3, 4, 5, 6, 7, 8, 9, 10 or more constant domains or portions thereof. The immunoglobulin region may comprise an amino acid sequence comprising 10, 20, 30, 40, 50, 60, 70, 80, 90, 100, 120, 140, 160, 180, 200, 225, 250, 275, 300, 350, 400, 500, 600, 700, 800, 900, 1000, 1200, 1400 or more amino acids based on or derived from an amino acid sequence of one or more constant domains. The amino acids may be consecutive. The amino acids may be non-consecutive.

The immunoglobulin region may comprise at least a portion of a complementarity-determining region (CDR). The immunoglobulin region may comprise one or more complementarity-determining regions (CDRs) or portions thereof. The immunoglobulin region may comprise 2, 3, 4, 5 or more complementarity-determining regions (CDRs) or portions thereof. The immunoglobulin region may comprise 6, 7, 8 or more complementarity-determining regions (CDRs) or portions thereof. The immunoglobulin region may comprise four or more complementarity-determining regions (CDRs) or portions thereof. The immunoglobulin region may comprise 9, 10, 11 or more complementarity-determining regions (CDRs) or portions thereof. The one or more CDRs may be CDR1, CDR2, CDR3 or a combination thereof. The one or more CDRs may be CDR1. The one or more CDRs may be CDR2. The one or more CDRs may be CDR3. The CDR may be a heavy chain CDR. The one or more CDRs may be a light chain CDR.

The immunoglobulin region may comprise an amino acid sequence comprising 1, 2, 3, 4, 5, 6, 7, 8, 9, 10 or more amino acids based on or derived from an amino acid sequence of a CDR. The immunoglobulin region may comprise an amino acid sequence comprising 3 or more amino acids based on or derived from an amino acid sequence of a CDR. The immunoglobulin region may comprise an amino acid sequence comprising 5 or more amino acids based on or derived from an amino acid sequence of a CDR. The immunoglobulin region may comprise an amino acid sequence comprising 10 or more amino acids based on or derived from an amino acid sequence of a CDR. The amino acids may be consecutive. The amino acids may be non-consecutive.

The immunoglobulin region may be based on or derived from at least a portion of an anti-T cell receptor immunoglobulin. The immunoglobulin region may be based on or derived from at least a portion of an anti-B cell receptor immunoglobulin.

The immunoglobulin region may be based on or derived from at least a portion of an anti-T cell co-receptor immunoglobulin. The immunoglobulin region may be based on or derived from at least a portion of an anti-CD3 immunoglobulin. The immunoglobulin region may be based on or derived from an anti-CD3 immunoglobulin. The anti-CD3 immunoglobulin may be UCHT1. The immunoglobulin region may be based on or derived from at least a portion of a Fab fragment of an anti-CD3 immunoglobulin. The immunoglobulin region may be based on or derived from an immunoglobulin fragment of an anti-CD3 immunoglobulin.

The immunoglobulin region may be based on or derived from an immunoglobulin or immunoglobulin fragment that binds to at least a portion of a receptor on a cell. The immunoglobulin region may be based on or derived from an immunoglobulin or immunoglobulin fragment that binds to at least a portion of a co-receptor on a cell. The immunoglobulin region may be based on or derived from an immunoglobulin or immunoglobulin fragment that binds to at least a portion of an antigen or cell surface marker on a cell. The cell may be a hematopoietic cell. The hematopoietic cell may be a myeloid cell. The myeloid cell may be an erythrocyte, thrombocyte, neutrophil, monocyte, macrophage, eosinophil, basophil, or mast cell. The hematopoietic cell may be a lymphoid cell. The lymphoid cell may be a B-cell, T-cell, or NK-cell. The hematopoietic cell may be a leukocyte. The hematopoietic cell may be a lymphocyte.

The immunoglobulin region may be based on or derived from an immunoglobulin or immunoglobulin fragment that binds to at least a portion of a receptor on a T-cell. The receptor may be a T-cell receptor (TCR). The TCR may comprise TCR alpha, TCR beta, TCR gamma and/or TCR delta. The receptor may be a T-cell receptor zeta.

The immunoglobulin region may be based on or derived from an immunoglobulin or immunoglobulin fragment that binds to at least a portion of a receptor on a lymphocyte, B-cell, macrophage, monocytes, neutrophils and/or NK cells. The receptor may be an Fc receptor. The Fc receptor may be an Fc-gamma receptor, Fc-alpha receptor and/or Fc-epsilon receptor. Fc-gamma receptors include, but are not limited to, FcγRI (CD64), FcγRIIA (CD32), FcγRIIB (CD32), FcγRIIIA (CD16a) and FcγRIIIB (CD16b). Fc-alpha receptors include, but are not limited to, FcαRI. Fc-epsilon receptors include, but are not limited to, FcεRI and FcεRII. The receptor may be CD89 (Fc fragment of IgA receptor or FCAR).

The immunoglobulin region may be based on or derived from an immunoglobulin or immunoglobulin fragment that binds at least a portion of a co-receptor on a T-cell. The co-receptor may be a CD3, CD4, and/or CD8. The immunoglobulin region may be based on or derived from an immunoglobulin fragment that binds to a CD3 co-receptor. The CD3 co-receptor may comprise CD3-gamma, CD3-delta and/or CD3-epsilon. CD8 may comprise CD8-alpha and/or CD8-beta chains.

In some embodiments, the immunoglobulin region is not specific for a mammalian target. In some embodiments, the immunoglobulin is an anti-viral immunoglobulin. In some embodiments, the immunoglobulin is an anti-bacterial immunoglobulin. In some embodiments, the immunoglobulin is an anti-parasitic immunoglobulin. In some embodiments, the immunoglobulin is an anti-fungal immunoglobulin. In some embodiments, the immunoglobulin region is derived from an immunoglobulin vaccine.

In some embodiments, the immunoglobulin region is based on or derived from immunoglobulins including, but not limited to, actoxumab, bezlotoxumab, CR6261, edobacomab, efungumab, exbivirumab, felvizumab, foravirumab, ibalizumab (TMB-355, TNX-355), libivirumab, motavizumab, nebacumab, pagibaximab, palivizumab, panobacumab, rafivirumab, raxibacumab, regavirumab, sevirumab (MSL-109), suvizumab, tefibazumab, tuvirumab, and urtoxazumab.

In some embodiments, the immunoglobulin region is based on or derived from immunoglobulins targeting Clostridium difficile, Orthomyxoviruses (Influenzavirus A, Influenzavirus B, Influenzavirus C, Isavirus, Thogotovirus), Escherichia coli, Candida, Rabies, Human Immunodeficiency Virus, Hepatitis, Staphylococcus, Respiratory Syncytial Virus, Pseudomonas aeruginosa, Bacillus anthracis, Cytomegalovirus, or Staphylococcus aureus.

The immunoglobulin region may be based on or derived from an anti-viral immunoglobulin. The anti-viral immunoglobulin may be directed against an epitope of a viral protein. The anti-bacterial immunoglobulin may target one or more viruses including, but not limited to, Adenoviruses, Herpesviruses, Poxviruses, Parvoviruses, Reoviruses, Picornaviruses, Togaviruses, Orthomyxoviruses, Rhabdoviruses, Retroviruses and Hepadnaviruses. The viral protein may be from a respiratory syncytial virus. The viral protein may be an F protein of the respiratory syncytiral virus. The epitope may be in the A antigenic site of the F protein. The anti-viral immunoglobulin may be based on or derived from palivizumab. The immunoglobulin may be based on or derived from an anti-viral vaccine. The anti-viral immunoglobulin may be based on or derived from exbivirumab, foravirumab, libivirumab, rafivirumab, regavirumab, sevirumab, tuvirumab, felvizumab, motavizumab, palivizumab, and/or suvizumab.

The immunoglobulin region may be based on or derived from an anti-viral immunoglobulin G. The immunoglobulin region may comprise at least a portion of an anti-viral immunoglobulin G. The immunoglobulin region may comprise an amino acid sequence that is at least about 50% homologous to at least a portion of an anti-viral immunoglobulin G. The immunoglobulin region may comprise an amino acid sequence that is at least about 60%, 65%, 70%, 75%, 80%, 85%, 90%, 92%, 95%, or 97% or more homologous to at least a portion of an anti-viral immunoglobulin G. The immunoglobulin region may comprise an amino acid sequence that is at least about 70% homologous to at least a portion of an anti-viral immunoglobulin G. The immunoglobulin region may comprise an amino acid sequence that is at least about 80% homologous to at least a portion of an anti-viral immunoglobulin G. In some embodiments the immunoglobulin region comprises an amino acid sequence based on or derived from an anti-viral immunoglobulin M.

The immunoglobulin region may comprise an amino acid sequence that comprises 10, 20, 30, 40, 50, 60, 70, 80, 90 or more amino acids of an anti-viral immunoglobulin G sequence. The immunoglobulin region may comprise an amino acid sequence that comprises 100, 200, 300, 400, 500, 600, 700, 800, 900 or more amino acids of an anti-viral immunoglobulin G sequence. The immunoglobulin region may comprise an amino acid sequence that comprises 50 or more amino acids of an anti-viral immunoglobulin G sequence. The immunoglobulin region may comprise an amino acid sequence that comprises 100 or more amino acids of an anti-viral immunoglobulin G sequence. The immunoglobulin region may comprise an amino acid sequence that comprises 200 or more amino acids of an anti-viral immunoglobulin G sequence.

The immunoglobulin region may be based on or derived from a palivizumab immunoglobulin. The immunoglobulin region may comprise at least a portion of a palivizumab immunoglobulin. The immunoglobulin region may comprise an amino acid sequence that is at least about 50% homologous to at least a portion of a palivizumab immunoglobulin. The immunoglobulin region may comprise an amino acid sequence that is at least about 60%, 65%, 70%, 75%, 80%, 85%, 90%, 92%, 95%, or 97% or more homologous to at least a portion of a palivizumab immunoglobulin. The immunoglobulin region may comprise an amino acid sequence that is at least about 70% homologous to at least a portion of a palivizumab immunoglobulin. The immunoglobulin region may comprise an amino acid sequence that is at least about 80% homologous to at least a portion of a palivizumab immunoglobulin.

The immunoglobulin region may comprise an amino acid sequence that comprises 10, 20, 30, 40, 50, 60, 70, 80, 90 or more amino acids of a palivizumab immunoglobulin sequence. The immunoglobulin region may comprise an amino acid sequence that comprises 100, 200, 300, 400, 500, 600, 700, 800, 900 or more amino acids of a palivizumab immunoglobulin sequence. The immunoglobulin region may comprise an amino acid sequence that comprises 50 or more amino acids of a palivizumab immunoglobulin sequence. The immunoglobulin region may comprise an amino acid sequence that comprises 100 or more amino acids of a palivizumab immunoglobulin sequence. The immunoglobulin region may comprise an amino acid sequence that comprises 200 or more amino acids of a palivizumab immunoglobulin sequence.

The immunoglobulin region may be based on or derived from an exbivirumab, foravirumab, libivirumab, rafivirumab, regavirumab, sevirumab, tuvirumab, felvizumab, motavizumab, palivizumab, and/or suvizumab immunoglobulin. The immunoglobulin region may comprise at least a portion of an exbivirumab, foravirumab, libivirumab, rafivirumab, regavirumab, sevirumab, tuvirumab, felvizumab, motavizumab, palivizumab, and/or suvizumab immunoglobulin. The immunoglobulin region may comprise an amino acid sequence that is at least about 50% homologous to at least a portion of an exbivirumab, foravirumab, libivirumab, rafivirumab, regavirumab, sevirumab, tuvirumab, felvizumab, motavizumab, palivizumab, and/or suvizumab immunoglobulin. The immunoglobulin region may comprise an amino acid sequence that is at least about 60%, 65%, 70%, 75%, 80%, 85%, 90%, 92%, 95%, or 97% or more homologous to at least a portion of an exbivirumab, foravirumab, libivirumab, rafivirumab, regavirumab, sevirumab, tuvirumab, felvizumab, motavizumab, palivizumab, and/or suvizumab immunoglobulin. The immunoglobulin region may comprise an amino acid sequence that is at least about 70% homologous to at least a portion of an exbivirumab, foravirumab, libivirumab, rafivirumab, regavirumab, sevirumab, tuvirumab, felvizumab, motavizumab, palivizumab, and/or suvizumab immunoglobulin. The immunoglobulin region may comprise an amino acid sequence that is at least about 80% homologous to at least a portion of an exbivirumab, foravirumab, libivirumab, rafivirumab, regavirumab, sevirumab, tuvirumab, felvizumab, motavizumab, palivizumab, and/or suvizumab immunoglobulin.

The immunoglobulin region may comprise an amino acid sequence that comprises 10, 20, 30, 40, 50, 60, 70, 80, 90 or more amino acids of an exbivirumab, foravirumab, libivirumab, rafivirumab, regavirumab, sevirumab, tuvirumab, felvizumab, motavizumab, palivizumab, and/or suvizumab immunoglobulin sequence. The immunoglobulin region may comprise an amino acid sequence that comprises 100, 200, 300, 400, 500, 600, 700, 800, 900 or more amino acids of an exbivirumab, foravirumab, libivirumab, rafivirumab, regavirumab, sevirumab, tuvirumab, felvizumab, motavizumab, palivizumab, and/or suvizumab immunoglobulin sequence. The immunoglobulin region may comprise an amino acid sequence that comprises 50 or more amino acids of an exbivirumab, foravirumab, libivirumab, rafivirumab, regavirumab, sevirumab, tuvirumab, felvizumab, motavizumab, palivizumab, and/or suvizumab immunoglobulin sequence. The immunoglobulin region may comprise an amino acid sequence that comprises 100 or more amino acids of an exbivirumab, foravirumab, libivirumab, rafivirumab, regavirumab, sevirumab, tuvirumab, felvizumab, motavizumab, palivizumab, and/or suvizumab immunoglobulin sequence. The immunoglobulin region may comprise an amino acid sequence that comprises 200 or more amino acids of an exbivirumab, foravirumab, libivirumab, rafivirumab, regavirumab, sevirumab, tuvirumab, felvizumab, motavizumab, palivizumab, and/or suvizumab immunoglobulin sequence.

The immunoglobulin region may be based on or derived from an anti-bacterial immunoglobulin. The anti-bacterial immunoglobulin may be directed against an epitope of a bacterial protein. The anti-bacterial immunoglobulin may target bacteria including, but not limited to, Acetobacter aurantius, Agrobacterium radiobacter, Anaplasma phagocytophilum, Azorhizobium caulinodans, Bacillus anthracis, Bacillus brevis, Bacillus cereus, Bacillus subtilis, Bacteroides fragilis, Bacteroides gingivalis, Bacteroides melaninogenicus, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacia, Calymmatobacterium granulomatis, Campylobacter coli, Campylobacter fetus, Campylobacter jejuni, Campylobacter pylori, Chlamydia trachomatis, Chlamydophila pneumoniae, Chlamydophila psittaci, Clostridium botulinum, Clostridium difficile, Corynebacterium diphtherias, Corynebacterium fusiforme, Coxiella burnetii, Enterobacter cloacae, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus influenzae, Haemophilus parainfluenzae, Haemophilus pertussis, Haemophilus vaginalis, Helicobacter pylori, Klebsiella pneumoniae, Lactobacillus acidophilus, Lactococcus lactis, Legionella pneumophila, Listeria monocytogenes, Methanobacterium extroquens, Microbacterium multiforme, Micrococcus luteus, Moraxella catarrhalis, Mycobacterium phlei, Mycobacterium smegmatis, Mycobacterium tuberculosis, Mycoplasma genitalium, Mycoplasma hominis, Mycoplasma pneumonic, Neisseria gonorrhoeae, Neisseria meningitidis, Pasteurella multocida, Pasteurella tularensis, Peptostreptococcus, Porphyromonas gingivalis, Prevotella melaninogenica, Pseudomonas aeruginosa, Rhizobium radiobacter, Rickettsia rickettsii, Rothia dentocariosa, Salmonella enteritidis, Salmonella typhi, Salmonella typhimurium, Shigella dysenteriae, Staphylococcus aureus, Staphylococcus epidermidis, Stenotrophomonas maltophilia, Streptococcus pneumoniae, Streptococcus pyogenes, Treponema pallidum, Treponema denticola, Vibrio cholerae, Vibrio comma, Vibrio parahaemolyticus, Vibrio vulnificus, Yersinia enterocolitica and Yersinia pseudotuberculosis. The immunoglobulin may be based on or derived from a bacterial vaccine. The anti-viral immunoglobulin may be based on or derived from nebacumab, panobacumab, raxibacumab, edobacomab, pagibaximab, and/or tefibazumab.

The immunoglobulin region may be based on or derived from an anti-bacterial immunoglobulin G. The immunoglobulin region may comprise at least a portion of an anti-bacterial immunoglobulin G. The immunoglobulin region may comprise an amino acid sequence that is at least about 50% homologous to at least a portion of an anti-bacterial immunoglobulin G. The immunoglobulin region may comprise an amino acid sequence that is at least about 60%, 65%, 70%, 75%, 80%, 85%, 90%, 92%, 95%, or 97% or more homologous to at least a portion of an anti-bacterial immunoglobulin G. The immunoglobulin region may comprise an amino acid sequence that is at least about 70% homologous to at least a portion of an anti-bacterial immunoglobulin G. The immunoglobulin region may comprise an amino acid sequence that is at least about 80% homologous to at least a portion of an anti-bacterial immunoglobulin G. In some embodiments the immunoglobulin region comprises an amino acid sequence based on or derived from an anti-viral immunoglobulin M.

The immunoglobulin region may comprise an amino acid sequence that comprises 10, 20, 30, 40, 50, 60, 70, 80, 90 or more amino acids of an anti-bacterial immunoglobulin G sequence. The immunoglobulin region may comprise an amino acid sequence that comprises 100, 200, 300, 400, 500, 600, 700, 800, 900 or more amino acids of an anti-bacterial immunoglobulin G sequence. The immunoglobulin region may comprise an amino acid sequence that comprises 50 or more amino acids of an anti-bacterial immunoglobulin G sequence. The immunoglobulin region may comprise an amino acid sequence that comprises 100 or more amino acids of an anti-bacterial immunoglobulin G sequence. The immunoglobulin region may comprise an amino acid sequence that comprises 200 or more amino acids of an anti-bacterial immunoglobulin G sequence.

The immunoglobulin region may be based on or derived from a Nebacumab, Panobacumab, Raxibacumab, Edobacomab, Pagibaximab, and/or Tefibazumab immunoglobulin. The immunoglobulin region may comprise at least a portion of a nebacumab, panobacumab, raxibacumab, edobacomab, pagibaximab, and/or tefibazumab immunoglobulin. The immunoglobulin region may comprise an amino acid sequence that is at least about 50% homologous to at least a portion of a nebacumab, panobacumab, raxibacumab, edobacomab, pagibaximab, and/or tefibazumab immunoglobulin. The immunoglobulin region may comprise an amino acid sequence that is at least about 60%, 65%, 70%, 75%, 80%, 85%, 90%, 92%, 95%, or 97% or more homologous to at least a portion of a nebacumab, panobacumab, raxibacumab, edobacomab, pagibaximab, and/or tefibazumab immunoglobulin. The immunoglobulin region may comprise an amino acid sequence that is at least about 70% homologous to at least a portion of a nebacumab, panobacumab, raxibacumab, edobacomab, pagibaximab, and/or tefibazumab immunoglobulin. The immunoglobulin region may comprise an amino acid sequence that is at least about 80% homologous to at least a portion of a nebacumab, panobacumab, raxibacumab, edobacomab, pagibaximab, and/or tefibazumab immunoglobulin.

The immunoglobulin region may comprise an amino acid sequence that comprises 10, 20, 30, 40, 50, 60, 70, 80, 90 or more amino acids of a nebacumab, panobacumab, raxibacumab, edobacomab, pagibaximab, and/or tefibazumab immunoglobulin sequence. The immunoglobulin region may comprise an amino acid sequence that comprises 100, 200, 300, 400, 500, 600, 700, 800, 900 or more amino acids of a nebacumab, panobacumab, raxibacumab, edobacomab, pagibaximab, and/or tefibazumab immunoglobulin sequence. The immunoglobulin region may comprise an amino acid sequence that comprises 50 or more amino acids of a nebacumab, panobacumab, raxibacumab, edobacomab, pagibaximab, and/or tefibazumab immunoglobulin sequence. The immunoglobulin region may comprise an amino acid sequence that comprises 100 or more amino acids of a nebacumab, panobacumab, raxibacumab, edobacomab, pagibaximab, and/or tefibazumab immunoglobulin sequence. The immunoglobulin region may comprise an amino acid sequence that comprises 200 or more amino acids of a nebacumab, panobacumab, raxibacumab, edobacomab, pagibaximab, and/or tefibazumab immunoglobulin sequence.

The immunoglobulin region may be based on or derived from an anti-parasitic immunoglobulin. The anti-parasitic immunoglobulin may be directed against an epitope of a parasite protein. The anti-parasitic immunoglobulin may target parasites or parasite proteins including, but not limited to parasites Acanthamoeba, Balamuthia mandrillaris, Babesia (B. divergens, B. bigemina, B. equi, B. microfti, B. duncani), Balantidium coli, Blastocystis, Cryptosporidium, Dientamoeba fragilis, Entamoeba histolytica, Giardia lamblia, Isospora belli, Leishmania, Naegleria fowleri, Plasmodium falciparum, Plasmodium vivax, Plasmodium ovale curtisi, Plasmodium ovale wallikeri, Plasmodium malariae, Plasmodium knowlesi, Rhinosporidium seeberi, Sarcocystis bovihominis, Sarcocystis suihominis, Toxoplasma gondii, Trichomonas vaginalis, Trypanosoma brucei, Trypanosoma cruzi, Cestoda, Taenia multiceps, Diphyllobothrium latum, Echinococcus granulosus, Echinococcus multilocularis, Echinococcus vogeli, Echinococcus oligarthrus, Hymenolepis nana, Hymenolepis diminuta, Taenia saginata, Taenia solium, Bertiella mucronata, Bertiella studeri, Spirometra erinaceieuropaei, Clonorchis sinensis; Clonorchis viverrini, Dicrocoelium dendriticum, Fasciola hepatica, Fasciola gigantica, Fasciolopsis buski, Gnathostoma spinigerum, Gnathostoma hispidum, Metagonimus yokogawai, Opisthorchis viverrini, Opisthorchis felineus, Clonorchis sinensis, Paragonimus westermani; Paragonimus africanus; Paragonimus caliensis; Paragonimus kellicotti; Paragonimus skrjabini; Paragonimus uterobilateralis, Schistosoma sp., Schistosoma mansoni, Schistosoma haematobium, Schistosoma japonicum, Schistosoma mekongi, Echinostoma echinatum, Trichobilharzia regenti, Schistosomatidae, Ancylostoma duodenale, Necator americanus, Angiostrongylus costaricensis, Anisakis, Ascaris sp. Ascaris lumbricoides, Baylisascaris procyonis, Brugia malayi, Brugia timori, Dioctophyme renale, Dracunculus medinensis, Enterobius vermicularis, Enterobius gregorii, Halicephalobus gingivalis, Loa filaria, Mansonella streptocerca, Onchocerca volvulus, Strongyloides stercoralis, Thelazia californiensis, Thelazia callipaeda, Toxocara canis, Toxocara cati, Trichinella spiralis, Trichinella britovi, Trichinella nelsoni, Trichinella nativa, Trichuris trichiura, Trichuris vulpis, Wuchereria bancrofti, Archiacanthocephala, Moniliformis moniliformis, Linguatula serrata, Oestroidea, Calliphoridae, Sarcophagidae, Tunga penetrans, Dermatobia hominis, Ixodidae, Argasidae, Cimex lectularius, Pediculus humanus, Pediculus humanus corporis, Pthirus pubis, Demodex folliculorum/brevis/canis, Sarcoptes scabiei, Cochliomyia hominivorax, and Pulex irritans.

The immunoglobulin region may be based on or derived from an anti-parasitic immunoglobulin G. The immunoglobulin region may comprise at least a portion of an anti-parasitic immunoglobulin G. The immunoglobulin region may comprise an amino acid sequence that is at least about 50% homologous to at least a portion of an anti-parasitic immunoglobulin G. The immunoglobulin region may comprise an amino acid sequence that is at least about 60%, 65%, 70%, 75%, 80%, 85%, 90%, 92%, 95%, or 97% or more homologous to at least a portion of an anti-parasitic immunoglobulin G. The immunoglobulin region may comprise an amino acid sequence that is at least about 70% homologous to at least a portion of an anti-parasitic immunoglobulin G. The immunoglobulin region may comprise an amino acid sequence that is at least about 80% homologous to at least a portion of an anti-parasitic immunoglobulin G. In some embodiments the immunoglobulin region comprises an amino acid sequence based on or derived from an anti-parasitic immunoglobulin M.

The immunoglobulin region may comprise an amino acid sequence that comprises 10, 20, 30, 40, 50, 60, 70, 80, 90 or more amino acids of an anti-parasitic immunoglobulin G sequence. The immunoglobulin region may comprise an amino acid sequence that comprises 100, 200, 300, 400, 500, 600, 700, 800, 900 or more amino acids of an anti-parasitic immunoglobulin G sequence. The immunoglobulin region may comprise an amino acid sequence that comprises 50 or more amino acids of an anti-parasitic immunoglobulin G sequence. The immunoglobulin region may comprise an amino acid sequence that comprises 100 or more amino acids of an anti-parasitic immunoglobulin G sequence. The immunoglobulin region may comprise an amino acid sequence that comprises 200 or more amino acids of an anti-parasitic immunoglobulin G sequence.

The immunoglobulin region may be based on or derived from an anti-fungal immunoglobulin. The anti-bacterial immunoglobulin may be directed against an epitope of a fungal protein. The anti-fungal immunoglobulin may target fungi or fungal proteins including, but not limited to Cryptococcus neoformans, Cryptococcus gattii, Candida albicans, Candida tropicalis, Candida stellatoidea, Candida glabrata, Candida krusei, Candida parapsilosis, Candida guillermondii, Candida viswanathii, Candida lusitaniae, Rhodotorula mucilaginosa, Schizosaccharomyces pombe, Saccharomyces cerevisiae, Brettanomyces bruxellensis, Candida stellata, Schizosaccharomyces pombe, Torulaspora delbrueckii, Zygosaccharomyces bailii, Yarrowia hpolytica, Saccharomyces exiguus and Pichia pastoris. The anti-fungal immunoglobulin may be based on or derived from efungumab.

The immunoglobulin region may be based on or derived from an anti-fungal immunoglobulin G. The immunoglobulin region may comprise at least a portion of an anti-fungal immunoglobulin G. The immunoglobulin region may comprise an amino acid sequence that is at least about 50% homologous to at least a portion of an anti-fungal immunoglobulin G. The immunoglobulin region may comprise an amino acid sequence that is at least about 60%, 65%, 70%, 75%, 80%, 85%, 90%, 92%, 95%, or 97% or more homologous to at least a portion of an anti-fungal immunoglobulin G. The immunoglobulin region may comprise an amino acid sequence that is at least about 70% homologous to at least a portion of an anti-fungal immunoglobulin G. The immunoglobulin region may comprise an amino acid sequence that is at least about 80% homologous to at least a portion of an anti-fungal immunoglobulin G. In some embodiments the immunoglobulin region comprises an amino acid sequence based on or derived from an anti-fungal immunoglobulin M.

The immunoglobulin region may comprise an amino acid sequence that comprises 10, 20, 30, 40, 50, 60, 70, 80, 90 or more amino acids of an anti-fungal immunoglobulin G sequence. The immunoglobulin region may comprise an amino acid sequence that comprises 100, 200, 300, 400, 500, 600, 700, 800, 900 or more amino acids of an anti-fungal immunoglobulin G sequence. The immunoglobulin region may comprise an amino acid sequence that comprises 50 or more amino acids of an anti-fungal immunoglobulin G sequence. The immunoglobulin region may comprise an amino acid sequence that comprises 100 or more amino acids of an anti-fungal immunoglobulin G sequence. The immunoglobulin region may comprise an amino acid sequence that comprises 200 or more amino acids of an anti-fungal immunoglobulin G sequence.

The immunoglobulin region may be based on or derived from an efungumab immunoglobulin. The immunoglobulin region may comprise at least a portion of an efungumab immunoglobulin. The immunoglobulin region may comprise an amino acid sequence that is at least about 50% homologous to at least a portion of an efungumab immunoglobulin. The immunoglobulin region may comprise an amino acid sequence that is at least about 60%, 65%, 70%, 75%, 80%, 85%, 90%, 92%, 95%, or 97% or more homologous to at least a portion of an efungumab immunoglobulin. The immunoglobulin region may comprise an amino acid sequence that is at least about 70% homologous to at least a portion of an efungumab immunoglobulin. The immunoglobulin region may comprise an amino acid sequence that is at least about 80% homologous to at least a portion of an efungumab immunoglobulin.

The immunoglobulin region may comprise an amino acid sequence that comprises 10, 20, 30, 40, 50, 60, 70, 80, 90 or more amino acids of an efungumab immunoglobulin sequence. The immunoglobulin region may comprise an amino acid sequence that comprises 100, 200, 300, 400, 500, 600, 700, 800, 900 or more amino acids of an efungumab immunoglobulin sequence. The immunoglobulin region may comprise an amino acid sequence that comprises 50 or more amino acids of an efungumab immunoglobulin sequence. The immunoglobulin region may comprise an amino acid sequence that comprises 100 or more amino acids of an efungumab immunoglobulin sequence. The immunoglobulin region may comprise an amino acid sequence that comprises 200 or more amino acids of an efungumab immunoglobulin sequence.

The immunoglobulin region may be based on or derived from a trastuzumab immunoglobulin G immunoglobulin. The immunoglobulin region may comprise at least a portion of a trastuzumab immunoglobulin G immunoglobulin. The immunoglobulin region may comprise an amino acid sequence that is at least about 50% homologous to at least a portion of a trastuzumab immunoglobulin G immunoglobulin. The immunoglobulin region may comprise an amino acid sequence that is at least about 60%, 65%, 70%, 75%, 80%, 85%, 90%, 92%, 95%, or 97% or more homologous to at least a portion of a trastuzumab immunoglobulin G immunoglobulin. The immunoglobulin region may comprise an amino acid sequence that is at least about 70% homologous to at least a portion of a trastuzumab immunoglobulin G immunoglobulin. The immunoglobulin region may comprise an amino acid sequence that is at least about 80% homologous to at least a portion of a trastuzumab immunoglobulin G immunoglobulin.

The immunoglobulin region may comprise an amino acid sequence that comprises 10, 20, 30, 40, 50, 60, 70, 80, 90 or more amino acids of a trastuzumab immunoglobulin G immunoglobulin sequence. The immunoglobulin region may comprise an amino acid sequence that comprises 100, 200, 300, 400, 500, 600, 700, 800, 900 or more amino acids of a trastuzumab immunoglobulin G immunoglobulin sequence. The immunoglobulin region may comprise an amino acid sequence that comprises 50 or more amino acids of a trastuzumab immunoglobulin G immunoglobulin sequence. The immunoglobulin region may comprise an amino acid sequence that comprises 100 or more amino acids of a trastuzumab immunoglobulin G immunoglobulin sequence. The immunoglobulin region may comprise an amino acid sequence that comprises 200 or more amino acids of a trastuzumab immunoglobulin G immunoglobulin sequence.

The immunoglobulin region may be based on or derived from an anti-Her2 immunoglobulin. The immunoglobulin region may comprise at least a portion of an anti-Her2 immunoglobulin. The immunoglobulin region may comprise an amino acid sequence that is at least about 50% homologous to at least a portion of an anti-Her2 immunoglobulin. The immunoglobulin region may comprise an amino acid sequence that is at least about 60%, 65%, 70%, 75%, 80%, 85%, 90%, 92%, 95%, or 97% or more homologous to at least a portion of an anti-Her2 immunoglobulin. The immunoglobulin region may comprise an amino acid sequence that is at least about 70% homologous to at least a portion of an anti-Her2 immunoglobulin. The immunoglobulin region may comprise an amino acid sequence that is at least about 80% homologous to at least a portion of an anti-Her2 immunoglobulin.

The immunoglobulin region may comprise an amino acid sequence that comprises 10, 20, 30, 40, 50, 60, 70, 80, 90 or more amino acids of an anti-Her2 immunoglobulin sequence. The immunoglobulin region may comprise an amino acid sequence that comprises 100, 200, 300, 400, 500, 600, 700, 800, 900 or more amino acids of an anti-Her2 immunoglobulin sequence. The immunoglobulin region may comprise an amino acid sequence that comprises 50 or more amino acids of an anti-Her2 immunoglobulin sequence. The immunoglobulin region may comprise an amino acid sequence that comprises 100 or more amino acids of an anti-Her2 immunoglobulin sequence. The immunoglobulin region may comprise an amino acid sequence that comprises 200 or more amino acids of an anti-Her2 immunoglobulin sequence.

The immunoglobulin region may be based on or derived from an anti-CD47 immunoglobulin. The immunoglobulin region may comprise at least a portion of an anti-CD47 immunoglobulin. The immunoglobulin region may comprise an amino acid sequence that is at least about 50% homologous to at least a portion of an anti-CD47 immunoglobulin. The immunoglobulin region may comprise an amino acid sequence that is at least about 60%, 65%, 70%, 75%, 80%, 85%, 90%, 92%, 95%, or 97% or more homologous to at least a portion of an anti-CD47 immunoglobulin. The immunoglobulin region may comprise an amino acid sequence that is at least about 70% homologous to at least a portion of an anti-CD47 immunoglobulin. The immunoglobulin region may comprise an amino acid sequence that is at least about 80% homologous to at least a portion of an anti-CD47 immunoglobulin.

The immunoglobulin region may comprise an amino acid sequence that comprises 10, 20, 30, 40, 50, 60, 70, 80, 90 or more amino acids of an anti-CD47 immunoglobulin sequence. The immunoglobulin region may comprise an amino acid sequence that comprises 100, 200, 300, 400, 500, 600, 700, 800, 900 or more amino acids of an anti-CD47 immunoglobulin sequence. The immunoglobulin region may comprise an amino acid sequence that comprises 50 or more amino acids of an anti-CD47 immunoglobulin sequence. The immunoglobulin region may comprise an amino acid sequence that comprises 100 or more amino acids of an anti-CD47 immunoglobulin sequence. The immunoglobulin region may comprise an amino acid sequence that comprises 200 or more amino acids of an anti-CD47 immunoglobulin sequence.

The immunoglobulin region may be based on or derived from an anti-cancer immunoglobulin. Examples of anti-cancer immunoglobulin include, but are not limited to, abciximab, adalimumab, alemtuzumab, basiliximab, belimumab, bevacizumab, brentuximab, canakinumab, certolizumab, cetuximab, daclizumab, denosumab, eculizumab, efalizumab, gemtuzumab, golimumab, ibritumomab, infliximab, ipilimumab, muromonab-cd3, natalizumab, ofatumumab, omalizumab, palivizumab, panitumumab, ranibizumab, rituximab, tocilizumab, tositumomab, trastuzumab.

The immunoglobulin region may comprise at least a portion of a human immunoglobulin. The immunoglobulin region may comprise at least a portion of a humanized immunoglobulin. The immunoglobulin region may comprise at least a portion of a chimeric immunoglobulin. The immunoglobulin region may be based on or derived from a human immunoglobulin. The immunoglobulin region may be based on or derived from a humanized immunoglobulin. The immunoglobulin region may be based on or derived from a chimeric immunoglobulin. The immunoglobulin region may be based on or derived from a monoclonal immunoglobulin. The immunoglobulin region may be based on or derived from a polyclonal immunoglobulin. The immunoglobulin region may comprise at least a portion of an immunoglobulin from a mammal, avian, reptile, amphibian, or a combination thereof. The mammal may be a human. The mammal may be a non-human primate. The mammal may be a dog, cat, sheep, goat, cow, rabbit, or mouse.

The immunoglobulin region may comprise a sequence based on or derived from one or more immunoglobulin and/or immunoglobulin fragment sequences. The immunoglobulin region may comprise a sequence that is at least about 50%, 55%, 60%, 65%, 70%, 75%, 80%, 85%, 90%, 92%, 95%, 97%, 98%, 99% or more homologous to a sequence based on or derived from one or more immunoglobulin and/or immunoglobulin fragments. The immunoglobulin region may comprise a sequence that is at least about 70% homologous to a sequence based on or derived from one or more immunoglobulin and/or immunoglobulin fragments. The immunoglobulin region may comprise a sequence that is at least about 80% homologous to a sequence based on or derived from one or more immunoglobulin and/or immunoglobulin fragments. The immunoglobulin region may comprise a sequence that is at least about 90% homologous to a sequence based on or derived from one or more immunoglobulin and/or immunoglobulin fragments. The immunoglobulin region may comprise a sequence that is at least about 95% homologous to a sequence based on or derived from one or more immunoglobulin and/or immunoglobulin fragments. The sequence may be a peptide sequence. The sequence may be a nucleotide sequence.

The immunoglobulin region may comprise a peptide sequence that differs from a peptide sequence based on or derived from one or more immunoglobulin and/or immunoglobulin fragments by less than or equal to about 200, 150, 100, 90, 80, 70, 60, 50, 40, 30, 20, 17, 15, 12, 10, 8, 6, 5, 4 or fewer amino acids. The immunoglobulin region may comprise a peptide sequence that differs from a peptide sequence based on or derived from one or more immunoglobulin and/or immunoglobulin fragments by less than or equal to about 4 or fewer amino acids. The immunoglobulin region may comprise a peptide sequence that differs from a peptide sequence based on or derived from one or more immunoglobulin and/or immunoglobulin fragments by less than or equal to about 3 or fewer amino acids. The immunoglobulin region may comprise a peptide sequence that differs from a peptide sequence based on or derived from one or more immunoglobulin and/or immunoglobulin fragments by less than or equal to about 2 or fewer amino acids. The immunoglobulin region may comprise a peptide sequence that differs from a peptide sequence based on or derived from one or more immunoglobulin and/or immunoglobulin fragments by less than or equal to about 1 or fewer amino acids. The amino acids may be consecutive, nonconsecutive, or a combination thereof. For example, the immunoglobulin region may comprise a peptide sequence that differs from a peptide sequence based on or derived from one or more immunoglobulin and/or immunoglobulin fragments by less than about 3 consecutive amino acids. Alternatively, or additionally, the immunoglobulin region may comprise a peptide sequence that differs from a peptide sequence based on or derived from one or more immunoglobulin and/or immunoglobulin fragments by less than about 2 non-consecutive amino acids. In another example, the immunoglobulin region may comprise a peptide sequence that differs from a peptide sequence based on or derived from one or more immunoglobulin and/or immunoglobulin fragments by less than about 5 amino acids, wherein 2 of the amino acids are consecutive and 2 of the amino acids are non-consecutive.

The immunoglobulin region may comprise a nucleotide sequence that differs from a nucleotide sequence based on or derived from one or more antibodies and/or immunoglobulin fragments by less than or equal to about 500, 400, 300, 200, 100, 90, 80, 70, 60, 50, 40, 30, 25, 20, 19, 18, 17, 16, 15, 14, 13, 12, 11, 10, 9, 8, 7, 6, 5, 4 or fewer nucleotides or base pairs. The immunoglobulin region may comprise a nucleotide sequence that differs from a nucleotide sequence based on or derived from one or more immunoglobulin and/or immunoglobulin fragments by less than or equal to about 15 or fewer nucleotides or base pairs. The immunoglobulin region may comprise a nucleotide sequence that differs from a nucleotide sequence based on or derived from one or more immunoglobulin and/or immunoglobulin fragments by less than or equal to about 12 or fewer nucleotides or base pairs. The immunoglobulin region may comprise a nucleotide sequence that differs from a nucleotide sequence based on or derived from one or more immunoglobulin and/or immunoglobulin fragments by less than or equal to about 9 or fewer nucleotides or base pairs. The immunoglobulin region may comprise a nucleotide sequence that differs from a nucleotide sequence based on or derived from one or more immunoglobulin and/or immunoglobulin fragments by less than or equal to about 6 or fewer nucleotides or base pairs. The immunoglobulin region may comprise a nucleotide sequence that differs from a nucleotide sequence based on or derived from one or more immunoglobulin and/or immunoglobulin fragments by less than or equal to about 4 or fewer nucleotides or base pairs. The immunoglobulin region may comprise a nucleotide sequence that differs from a nucleotide sequence based on or derived from one or more immunoglobulin and/or immunoglobulin fragments by less than or equal to about 3 or fewer nucleotides or base pairs. The immunoglobulin region may comprise a nucleotide sequence that differs from a nucleotide sequence based on or derived from one or more immunoglobulin and/or immunoglobulin fragments by less than or equal to about 2 or fewer nucleotides or base pairs. The immunoglobulin region may comprise a nucleotide sequence that differs from a nucleotide sequence based on or derived from one or more immunoglobulin and/or immunoglobulin fragments by less than or equal to about 1 or fewer nucleotides or base pairs. The nucleotides or base pairs may be consecutive, nonconsecutive, or a combination thereof. For example, the immunoglobulin region may comprise a nucleotide sequence that differs from a nucleotide sequence based on or derived from one or more immunoglobulin and/or immunoglobulin fragments by less than about 3 consecutive nucleotides or base pairs. Alternatively, or additionally, the immunoglobulin region may comprise a nucleotide sequence that differs from a nucleotide sequence based on or derived from one or more immunoglobulin and/or immunoglobulin fragments by less than about 2 non-consecutive nucleotides or base pairs. In another example, the immunoglobulin region may comprise a nucleotide sequence that differs from a nucleotide sequence based on or derived from one or more immunoglobulin and/or immunoglobulin fragments by less than about 5 nucleotides or base pairs, wherein 2 of the nucleotides or base pairs are consecutive and 2 of the nucleotides or base pairs are non-consecutive.

The peptide sequence of the immunoglobulin region may differ from the peptide sequence of the immunoglobulin or immunoglobulin fragment that it is based on and/or derived from by one or more amino acid substitutions. The peptide sequence of the immunoglobulin region may differ from the peptide sequence of the immunoglobulin or immunoglobulin fragment that it is based on and/or derived from by two or more amino acid substitutions. The peptide sequence of the immunoglobulin region may differ from the peptide sequence of the immunoglobulin or immunoglobulin fragment that it is based on and/or derived from by three or more amino acid substitutions. The peptide sequence of the immunoglobulin region may differ from the peptide sequence of the immunoglobulin or immunoglobulin fragment that it is based on and/or derived from by four or more amino acid substitutions. The peptide sequence of the immunoglobulin region may differ from the peptide sequence of the immunoglobulin or immunoglobulin fragment that it is based on and/or derived from by five or more amino acid substitutions. The peptide sequence of the immunoglobulin region may differ from the peptide sequence of the immunoglobulin or immunoglobulin fragment that it is based on and/or derived from by six or more amino acid substitutions. The peptide sequence of the immunoglobulin region may differ from the peptide sequence of the immunoglobulin or immunoglobulin fragment that it is based on and/or derived from by 1, 2, 3, 4, 5, 6, 7, 8, 9, 10, 12, 14, 15, 17, 20, 25 or more amino acid substitutions. The peptide sequence of the immunoglobulin region may differ from the peptide sequence of the immunoglobulin or immunoglobulin fragment that it is based on and/or derived from by about 20-30, 30-40, 40-50, 50-60, 60-70, 80-90, 90-100, 100-150, 150-200, 200-300 or more amino acid substitutions.

The nucleotide sequence of the immunoglobulin region may differ from the nucleotide sequence of the immunoglobulin or immunoglobulin fragment that it is based on and/or derived from by one or more nucleotide and/or base pair substitutions. The nucleotide sequence of the immunoglobulin region may differ from the nucleotide sequence of the immunoglobulin or immunoglobulin fragment that it is based on and/or derived from by two or more nucleotide and/or base pair substitutions. The nucleotide sequence of the immunoglobulin region may differ from the nucleotide sequence of the immunoglobulin or immunoglobulin fragment that it is based on and/or derived from by three or more nucleotide and/or base pair substitutions. The nucleotide sequence of the immunoglobulin region may differ from the nucleotide sequence of the immunoglobulin or immunoglobulin fragment that it is based on and/or derived from by four or more nucleotide and/or base pair substitutions. The nucleotide sequence of the immunoglobulin region may differ from the nucleotide sequence of the immunoglobulin or immunoglobulin fragment that it is based on and/or derived from by five or more nucleotide and/or base pair substitutions. The nucleotide sequence of the immunoglobulin region may differ from the nucleotide sequence of the immunoglobulin or immunoglobulin fragment that it is based on and/or derived from by six or more nucleotide and/or base pair substitutions. The nucleotide sequence of the immunoglobulin region may differ from the nucleotide sequence of the immunoglobulin or immunoglobulin fragment that it is based on and/or derived from by nine or more nucleotide and/or base pair substitutions. The nucleotide sequence of the immunoglobulin region may differ from the nucleotide sequence of the immunoglobulin or immunoglobulin fragment that it is based on and/or derived from by twelve or more nucleotide and/or base pair substitutions. The nucleotide sequence of the immunoglobulin region may differ from the nucleotide sequence of the immunoglobulin or immunoglobulin fragment that it is based on and/or derived from by fifteen or more nucleotide and/or base pair substitutions. The nucleotide sequence of the immunoglobulin region may differ from the nucleotide sequence of the immunoglobulin or immunoglobulin fragment that it is based on and/or derived from by eighteen or more nucleotide and/or base pair substitutions. The nucleotide sequence of the immunoglobulin region may differ from the nucleotide sequence of the immunoglobulin or immunoglobulin fragment that it is based on and/or derived from by 20, 22, 24, 25, 27, 30 or more nucleotide and/or base pair substitutions. The nucleotide sequence of the immunoglobulin region may differ from the nucleotide sequence of the immunoglobulin or immunoglobulin fragment that it is based on and/or derived from by about 30-40, 40-50, 50-60, 60-70, 70-80, 80-90, 90-100, 100-200, 200-300, 300-400 or more nucleotide and/or base pair substitutions.

The immunoglobulin region may comprise at least about 10, 20, 30, 40, 50, 60, 70, 80, 90, 100 or more amino acids. The immunoglobulin region may comprise at least about 125, 150, 175, 200, 225, 250, 275, 300, 325, 350, 375, 400, 425, 450, 475, 500, 525, 550, 575, 600, 625, 650, 675, 700 or more amino acids. The immunoglobulin region may comprise at least about 100 amino acids. The immunoglobulin region may comprise at least about 200 amino acids. The immunoglobulin region may comprise at least about 400 amino acids. The immunoglobulin region may comprise at least about 500 amino acids. The immunoglobulin region may comprise at least about 600 amino acids.

The immunoglobulin region may comprise less than about 2000, 1900, 1800, 1700, 1600, 1500, 1400, 1300, 1200 or 1100 amino acids. The immunoglobulin region may comprise less than about 1000, 950, 900, 850, 800, 750, or 700 amino acids. The immunoglobulin region may comprise less than about 1500 amino acids. The immunoglobulin region may comprise less than about 1000 amino acids. The immunoglobulin region may comprise less than about 800 amino acids. The immunoglobulin region may comprise less than about 700 amino acids.

The immunoglobulin fusion protein may further comprise an immunoglobulin region comprising 30 or fewer consecutive amino acids of a complementarity determining region 3 (CDR3). The immunoglobulin region may comprise 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 or fewer consecutive amino acids of a CDR3. The immunoglobulin region may comprise 15 or fewer consecutive amino acids of a CDR3. The immunoglobulin region may comprise 14 or fewer consecutive amino acids of a CDR3. The immunoglobulin region may comprise 13 or fewer consecutive amino acids of a CDR3. The immunoglobulin region may comprise 12 or fewer consecutive amino acids of a CDR3. The immunoglobulin region may comprise 11 or fewer consecutive amino acids of a CDR3. The immunoglobulin region may comprise 10 or fewer consecutive amino acids of a CDR3. The immunoglobulin region may comprise 9 or fewer consecutive amino acids of a CDR3. The immunoglobulin region may comprise 8 or fewer consecutive amino acids of a CDR3. The immunoglobulin region may comprise 7 or fewer consecutive amino acids of a CDR3. The immunoglobulin region may comprise 6 or fewer consecutive amino acids of a CDR3. The immunoglobulin region may comprise 5 or fewer consecutive amino acids of a CDR3. The immunoglobulin region may comprise 4 or fewer consecutive amino acids of a CDR3. The immunoglobulin region may comprise 3 or fewer consecutive amino acids of a CDR3. The immunoglobulin region may comprise 2 or fewer consecutive amino acids of a CDR3. The immunoglobulin region may comprise 1 or fewer consecutive amino acids of a CDR3. In some instances, the immunoglobulin region does not contain a CDR3.

The immunoglobulin region may comprise an amino acid sequence that is based on or derived from any one of SEQ ID NOs: 5-8. The immunoglobulin region may comprise an amino acid sequence that is at least about 50% homologous to any one of SEQ ID NOs: 5-8. The immunoglobulin region may comprise an amino acid sequence that is at least about 60%, 65%, 70%, 75%, 80%, 85%, 90%, 95%, or 97% homologous to any one of SEQ ID NOs 5-8. The immunoglobulin region may comprise an amino acid sequence that is at least about 70% homologous to any one of SEQ ID NOs: 5-8. The immunoglobulin region may comprise an amino acid sequence that is at least about 80% homologous to any one of SEQ ID NOs: 5-8. The immunoglobulin region may comprise an amino acid sequence that is at least about 50% identical to any one of SEQ ID NOs: 5-8. The immunoglobulin region may comprise an amino acid sequence that is at least about 60%, 65%, 70%, 75%, 80%, 85%, 90%, 95%, or 97% identical to any one of SEQ ID NOs 5-8. The immunoglobulin region may comprise an amino acid sequence that is at least about 70% identical to any one of SEQ ID NOs: 5-8. The immunoglobulin region may comprise an amino acid sequence that is at least about 80% identical to any one of SEQ ID NOs: 5-8. The immunoglobulin region may comprise an amino acid sequence that is 100% identical to any one of SEQ ID NOs: 5-8. In some embodiments, the immunoglobulin region comprises an amino acid sequence that is at least about 50%, 55%, 60%, 65%, 70%, 75%, 80%, 85%, 90%, 95%, or 97% homologous to an amino acid sequence of any one of SEQ ID NOs: 5-8. In some embodiments, the immunoglobulin region comprises an amino acid sequence that is at least about 50%, 55%, 60%, 65%, 70%, 75%, 80%, 85%, 90%, 95%, or 97% identical to an amino acid sequence of any one of SEQ ID NOs: 5-8. The immunoglobulin region includes a Fab region that is based on or derived from a sequence from any one of SEQ ID NOs: 5-8. In some embodiments, the immunoglobulin region comprises an amino acid Fab sequence derived from a sequence that is at least about 70%, 80%, 80%, 90%, 95% or 100% to any one of SEQ ID NOs: 5-8.

The immunoglobulin region may comprise an amino acid sequence comprising 10, 20, 30, 40, 50, 60, 70, 80, 90, 100 or more amino acids based on or derived from any one of SEQ ID NOs: 5-8. The immunoglobulin region may comprise an amino acid sequence comprising 125, 150, 175, 200, 225, 250, 275, 300, 325, 350, 375, 400, 425, 450, 475, 450, 500 or more amino acids based on or derived from any one of SEQ ID NOs: 5-8. The immunoglobulin region may comprise an amino acid sequence comprising 10 or more amino acids based on or derived from any one of SEQ ID NOs: 5-8. The immunoglobulin region may comprise an amino acid sequence comprising 50 or more amino acids based on or derived from any one of SEQ ID NOs: 5-8. The immunoglobulin region may comprise an amino acid sequence comprising 100 or more amino acids based on or derived from any one of SEQ ID NOs: 5-8. The immunoglobulin region may comprise an amino acid sequence comprising 200 or more amino acids based on or derived from any one of SEQ ID NOs: 5-8. The amino acids may be consecutive. Alternatively, or additionally, the amino acids are nonconsecutive. In some embodiments, the immunoglobulin region may comprise amino acids derived from any one of SEQ ID NOs: 5-8 and amino acids not derived from any one of SEQ ID NOs: 5-8. In some embodiments, the immunoglobulin region may comprise amino acids derived from one or more of SEQ ID NOs: 5-8 and amino acids not derived from any one of SEQ ID NOs: 5-8. In some embodiments, the immunoglobulin region comprises amino acids derived from 1, 2, 3, or 4 of SEQ ID NOs: 5-8.

The immunoglobulin region may be encoded by a nucleotide sequence that is based on or derived from any one of SEQ ID NOs: 1-4. The immunoglobulin region may be encoded by a nucleotide sequence that is at least about 50% homologous to any one of SEQ ID NOs: 1-4. The immunoglobulin region may be encoded by a nucleotide sequence that is at least about 60%, 65%, 70%, 75%, 80%, 85%, 90%, 95%, or 97% homologous to any one of SEQ ID NOs: 1-4. The immunoglobulin region may be encoded by a nucleotide sequence that is at least about 70% homologous to any one of SEQ ID NOs: 1-4. The immunoglobulin region may be encoded by a nucleotide sequence that is at least about 80% homologous to any one of SEQ ID NOs: 1-4. The immunoglobulin region may be encoded by a nucleotide sequence that is at least about 50% identical to any one of SEQ ID NOs: 1-4. The immunoglobulin region may be encoded by a nucleotide sequence that is at least about 60%, 65%, 70%, 75%, 80%, 85%, 90%, 95%, or 97% identical to any one of SEQ ID NOs: 1-4. The immunoglobulin region may be encoded by a nucleotide sequence that is at least about 70% identical to any one of SEQ ID NOs: 1-4. The immunoglobulin region may be encoded by a nucleotide sequence that is at least about 80% identical to any one of SEQ ID NOs: 1-4. The immunoglobulin region may be encoded by a nucleotide sequence that is 100% identical to any one of SEQ ID NOs: 1-4. The immunoglobulin region includes a Fab region that is based on or derived from a sequence from any one of SEQ ID NOs: 1-4. In some embodiments, the immunoglobulin region comprises an amino acid Fab sequence derived from a sequence that is at least about 70%, 80%, 80%, 90%, 95% or 100% to any one of SEQ ID NOs: 1-4.

The immunoglobulin region may be encoded by a nucleotide sequence comprising 10, 20, 30, 40, 50, 60, 70, 80, 90, 100 or more nucleotides based on or derived from any one of SEQ ID NOs: 1-4. The immunoglobulin region may be encoded by a nucleotide sequence comprising 125, 150, 175, 200, 225, 250, 275, 300, 325, 350, 375, 400, 425, 450, 475, 450, 500 or more nucleotides based on or derived from any one of SEQ ID NOs: 1-4. The immunoglobulin region may be encoded by a nucleotide sequence comprising 600, 650, 700, 750, 800, 850, 900, 950, 1000 or more nucleotides based on or derived from any one of SEQ ID NOs: 1-4. The immunoglobulin region may be encoded by a nucleotide sequence comprising 1100, 1200, 1300, 1400, 1500 or more nucleotides based on or derived from any one of SEQ ID NOs: 1-4. The immunoglobulin region may be encoded by a nucleotide sequence comprising 100 or more nucleotides based on or derived from any one of SEQ ID NOs: 1-4. The immunoglobulin region may be encoded by a nucleotide sequence comprising 500 or more nucleotides based on or derived from any one of SEQ ID NOs: 1-4. The immunoglobulin region may be encoded by a nucleotide sequence comprising 1000 or more nucleotides based on or derived from any one of SEQ ID NOs: 1-4. The immunoglobulin region may be encoded by a nucleotide sequence comprising 1300 or more nucleotides based on or derived from any one of SEQ ID NOs: 1-4. The nucleotides may be consecutive. In some embodiments, the immunoglobulin region is encoded by a nucleotide sequence comprising nucleotides derived from any one of SEQ ID NOs: 1-4 and nucleotides not derived from any one of SEQ ID NOs: 1-4. In some embodiments, the immunoglobulin region is encoded by a nucleotide sequence comprising nucleotides derived from one or more of SEQ ID NOs: 1-4 and nucleotides not derived from any one of SEQ ID NOs: 1-4. In some embodiments, the immunoglobulin region is encoded by a nucleotide sequence derived from 1, 2, 3, or 4 of SEQ ID NOs: 1-4.

### Therapeutic Peptide

In one aspect of the disclosure, provided herein are immunoglobulin fusion proteins comprising a therapeutic peptide and an immunoglobulin region. The immunoglobulin fusion proteins may comprise two or more therapeutic peptides. The immunoglobulin fusion proteins disclosed herein may comprise 3, 4, 5, or more therapeutic peptides. The therapeutic peptide may be attached to an immunoglobulin region via a connecting peptide. In some embodiments, one or more additional therapeutic peptides are attached to the first or a second immunoglobulin region. The one or more therapeutic peptides may be attached to one or more immunoglobulin regions. The two or more therapeutic peptides may be attached to two or more immunoglobulin regions. The two or more therapeutic peptides may be attached to one or more immunoglobulin chains. The two or more therapeutic peptides may be attached to two or more immunoglobulin chains. The two or more therapeutic peptides may be attached to one or more units within the one or more immunoglobulin regions. The two or therapeutic peptides may be attached to two or more units within the one or more immunoglobulin regions. In some embodiments, the therapeutic peptide is connected to the immunoglobulin region without the aid of a connecting peptide.

The immunoglobulin fusion proteins disclosed herein may comprise one or more therapeutic agents. The therapeutic agent may be a peptide. The therapeutic agent may be a small molecule. The immunoglobulin fusion proteins disclosed herein may comprise two or more therapeutic agents. The immunoglobulin fusion proteins disclosed herein may comprise 3, 4, 5, 6 or more therapeutic agents. The two or more therapeutic agents may be the same. The two or more therapeutic agents may be different.

The therapeutic peptide may comprise any secondary structure, for example alpha helix or beta strand or comprise no regular secondary structure. The therapeutic peptide may comprise amino acids with one or more modifications including, but not limited to, myristoylation, palmitoylation, isoprenylation, glypiation, lipoylation, acylation, acetylation, aklylation, methylation, glycosylation, malonylation, hydroxylation, iodination, nucleotide addition, oxidation, phosphorylation, adenylylation, propionylation, succinylation, sulfation, selenoylation, biotinylation, pegylation, deimination, deamidation, eliminylation, and carbamylation. The therapeutic peptide may comprise one or more amino acids conjugated to one or more small molecules, for example a drug. In some embodiments, the therapeutic peptide comprises one or more non-natural amino acids. In some embodiments, the therapeutic peptide comprises 1, 2, 3, 4, 5, 6, 7, 8, 9, 10, 11, 12, 13, 14, 15, 16, 17, 18, 19, 20, 30, 40, 50 or more non-natural amino acids. In some embodiments, the therapeutic peptide comprises one or more amino acids substitutions. In some embodiments, the therapeutic peptide comprises 1, 2, 3, 4, 5, 6, 7, 8, 9, 10, 11, 12, 13, 14, 15, 16, 17, 18, 19, 20, 30, 40, 50 or more amino acid substitutions.

The therapeutic peptide may be inserted into the immunoglobulin region. Insertion of the therapeutic peptide into the immunoglobulin region may comprise removal or deletion of a portion of the immunoglobulin from which the immunoglobulin region is based on or derived from. The therapeutic peptide may replace at least a portion of a heavy chain. The therapeutic peptide may replace at least a portion of a light chain. The therapeutic peptide may replace at least a portion of a variable domain. The therapeutic peptide may replace at least a portion of a constant domain. The therapeutic peptide may replace at least a portion of a complementarity determining region (CDR). The therapeutic peptide may replace at least a portion of a CDR1. The therapeutic peptide may replace at least a portion of a CDR2. The therapeutic peptide may replace at least a portion of a CDR3. The therapeutic peptide may replace at least about 5%, 10%, 15%, 20%, 25%, 30%, 35%, 40%, 45%, 50%, 55%, 60%, 65%, 70%, 75%, 80%, 85%, 90%, 95%, or more of the immunoglobulin or a portion thereof. For example, the therapeutic peptide may replace at least about 50% of a variable domain. The therapeutic peptide may replace at least about 70% of a variable domain. The therapeutic peptide may replace at least about 80% of a variable domain. The therapeutic peptide may replace at least about 90% of a variable domain. The therapeutic peptide may replace at least about 95% of a variable domain. For example, the therapeutic peptide may replace at least about 50% of an amino terminus of an immunoglobulin region. The therapeutic peptide may replace at least about 70% of an amino terminus of an immunoglobulin region. The therapeutic peptide may replace at least about 80% of an amino terminus of an immunoglobulin region. The therapeutic peptide may replace at least about 90% of an amino terminus of an immunoglobulin region. The therapeutic peptide may replace at least about 95% of an amino terminus of an immunoglobulin region. The therapeutic peptide may replace at least about 50% of a CDR. The therapeutic peptide may replace at least about 70% of a CDR. The therapeutic peptide may replace at least about 80% of a CDR. The therapeutic peptide may replace at least about 90% of a CDR. The therapeutic peptide may replace at least about 95% of a CDR.

The one or more therapeutic peptides may be based on or derived from a protein. The protein may be a growth factor, cytokine, hormone or toxin. The growth factor may be GCSF, GMCSF, GDF11 or FGF21. The GCSF may be a bovine GCSF. The GCSF may be a human GCSF. The GMCSF may be a bovine GMCSF or a human GMCSF. The FGF21 may be a bovine FGF21. The FGF21 may be a human FGF21.

The cytokine may be an interferon or interleukin. The cytokine may be stromal cell-derived factor 1 (SDF-1). The interferon may be interferon-beta. The interferon may be interferon-alpha. The interleukin may be interleukin 11 (IL-11). The interleukin may be interleukin 8 (IL-8) or interleukin 21 (IL-21).

The hormone may be exendin-4, GLP-1, relaxin, oxyntomodulin, leptin, betatrophin, bovine growth hormone (bGH), human growth hormone (hGH), erythropoietin (EPO), or parathyroid hormone. The hormone may be somatostatin. The parathyroid hormone may be a human parathyroid hormone. The erythropoietin may be a human erythropoietin.

The toxin may be Moka1, VM-24, Mamba1, Amgen1, 550 peptide or protoxin2. The toxin may be ziconotide or chlorotoxin.

The protein may be angiopoeitin-like 3 (ANGPTL3). The angiopoeitin-like 3 may be a human angiopoeitin-like 3.

In some embodiments, one or more regions of the therapeutic peptide is configured to treat diabetes and/or diabetes related conditions. In some embodiments, 2, 3, 4, 5 or more regions of the therapeutic peptide are configured to treat diabetes and/or diabetes related conditions. Diabetes may include, type I diabetes, type 2 diabetes, gestational diabetes, and prediabetes. In some embodiments, one or more regions of the therapeutic peptide is configured to treat obesity and/or obesity related conditions. In some embodiments, 2, 3, 4, 5 or more regions of the therapeutic peptide are configured to treat obesity and/or obesity related conditions. Conditions may include complications and diseases. Examples of diabetes related conditions include, but are not limited to, diabetic retinopathy, diabetic nephropathy, diabetic heart disease, diabetic foot disorders, diabetic neuropathy, macrovascular disease, diabetic cardiomyopathy, infection and diabetic ketoacidosis. Diabetic neuropathy may include, but is not limited to symmetric polyneuropathy, autonomic neuropathy, radiculopathy, cranial neuropathy, and mononeuropathy. Obesity related conditions include, but are not limited to, heart disease, stroke, high blood pressure, diabetes, osteoarthritis, gout, sleep apnea, asthma, gallbladder disease, gallstones, abnormal blood fats (e.g., abnormal levels of LDL and HDL cholesterol), obesity hypoventilation syndrome, reproductive problems, hepatic steatosis, and mental health conditions.

In some embodiments, one or more regions of the therapeutic peptide is a glucagon-like protein-1 (GLP-1) receptor agonist or formulation thereof. In some embodiments, one or more regions of the therapeutic peptide is an incretin mimetic. In some embodiments, one or more regions of the therapeutic peptide comprises an amino acid sequence based on or derived from an amino acid sequence of exendin-4, exenatide, or synthetic thereof. In some embodiments, one or more regions of the therapeutic peptide is a glucagon analog or formulation thereof. In some embodiments, one or more regions of the therapeutic peptide comprises an amino acid sequence based on or derived from an amino acid sequence of insulin. In some embodiments, one or more regions of the therapeutic peptide is dual-specific. In some embodiments, the therapeutic peptide has specificity for a GLP-1 receptor and a glucagon receptor. In some embodiments, one or more regions of the therapeutic peptide comprises an amino acid sequence based on or derived from an amino acid sequence of oxyntomodulin.

In some embodiments, one or more regions of the therapeutic peptide is configured to treat short bowel syndrome and/or short bowel syndrome related conditions. In some embodiments, 2, 3, 4, 5 or more regions of the therapeutic peptide are configured to treat short bowel syndrome and/or short bowel syndrome related conditions. Short bowel syndrome related conditions may include, but are not limited to, bacterial overgrowth in the small intestine, metabolic acidosis, gallstones, kidney stones, malnutrition, osteomalacia, intestinal failure, and weight loss. In some embodiments, one or more regions of the therapeutic peptide is configured to treat inflammatory bowel disease and/or an inflammatory bowel related conditions. In some embodiments, 2, 3, 4, 5 or more regions of the therapeutic peptide are configured to treat inflammatory bowel disease and/or an inflammatory bowel related conditions. Inflammatory bowel disease and/or inflammatory bowel disease related conditions may include, but are not limited to, ulcerative colitis, Crohn's disease, collagenous colitis, lymphocytic colitis, ischaemic colitis, diversion colitis, Behcet's disease, intermediate colitis, anemia, arthritis, pyoderma gangrenosum, primary sclerosing cholangitis, non-thyroidal illness syndrome; and abdominal pain, vomiting, diarrhea, rectal bleeding, internal cramps or muscle spasms, and weight loss in individual with an inflammatory bowel disease. In some embodiments, an immunoglobulin fusion protein comprising a glucagon or a glucagon like peptide (e.g., GLP2, GLP2) is useful to treat inflammatory bowel disease and/or an inflammatory bowel disease condition. In some embodiments, an immunoglobulin fusion protein comprising an amino acid sequence that is at least about or at least about 50%, 60%, 70%, 80%, 85%, 86%, 87%, 88%, 89%, 90%, 91%, 92%, 93%, 94%, 95%, 96%, 97%, 98%, 99%, or 100% identical to any amino acid sequence of 69, 70, 193, 194, 195, 217, 218, 219, 220, and 221 is useful to treat inflammatory bowel disease. In some embodiments, an immunoglobulin fusion protein comprising a glucagon or a glucagon like peptide (e.g., GLP2, GLP2) is useful to treat short bowel syndrome and/or a short bowel syndrome condition. In some embodiments, an immunoglobulin fusion protein comprising an amino acid sequence that is at least about or at least about 50%, 60%, 70%, 80%, 85%, 86%, 87%, 88%, 89%, 90%, 91%, 92%, 93%, 94%, 95%, 96%, 97%, 98%, 99%, or 100% identical to any amino acid sequence of 69, 70, 193, 194, 195, 217, 218, 219, 220, and 221 is useful to treat short bowel syndrome.

In some embodiments, one or more regions of the therapeutic peptide comprises an amino acid sequence based on or derived from an amino acid sequence of glucagon, glucagon analog, glucagon like peptide, and/or a glucagon like peptide analog. In some embodiments, one or more regions of the therapeutic peptide comprises an amino acid sequence based on or derived from an amino acid sequence of a glucagon like peptide-2 (GLP-2).

In some embodiments, one or more regions of the therapeutic peptide is configured to treat an autoimmune disease and/or autoimmune disease related conditions. In some embodiments, 2, 3, 4, 5 or more regions of the therapeutic peptide are configured to treat autoimmune disease and/or autoimmune disease related conditions. Autoimmune disease and/or autoimmune disease related conditions may include, but are not limited to, acute disseminated encephalomyelitis, alopecia areata, antiphospholipid syndrome, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendrocrine syndrome, autoimmune progesterone dermatitis, autoimmune thrombocytopenic purpura, autoimmune urticaria, autoimmune uveitis, Behcet's disease, Celiac disease, cold agglutinin disease, Crohn's disease, dermatomyositis, diabetes mellitus type 1, eosinophilic fasciitis, gastrointestinal pemphigoid, Goodpasture's syndrome, Grave's disease, Guillain-Barre syndrome, Hashimoto's encephalopathy, Hashimoto's thyroiditis, idiopathic thrombocytopenic purpura, lupus erythematosus, Miller-Fisher syndrome, mixed connective tissue disease, multiple sclerosis, myasthenia gravis, narcolepsy, pemphigus vulgaris, pernicious anaemia, polymyositis, primary biliary cirrhosis, psoriasis, psoriatic arthritis, relapsing polychondritis, rheumatoid arthritis, rheumatic fever, Sjogren's syndrome, temporal arteritis, transverse myelitis, ulcerative colitis, undifferentiated connective tissue disease, vasculitis, and Wegener's granulomatosis.

In some embodiments, one or more regions of the therapeutic peptide comprises an amino acid sequence based on or derived from an amino acid sequence which binds to potassium channels. In some embodiments, one or more regions of the therapeutic peptide comprises an amino acid sequence based on or derived from an amino acid sequence of a Mokatoxin-1 (Moka).

In some embodiments, one or more regions of the therapeutic peptide is configured to treat pain. In some embodiments, 2, 3, 4, 5 or more regions of the therapeutic peptide are configured to treat pain.

In some embodiments, one or more regions of the therapeutic peptide comprises an amino acid sequence based on or derived from an amino acid sequence which is a neurotoxin. In some embodiments, one or more regions of the therapeutic peptide comprises an amino acid sequence based on or derived from an amino acid sequence of a neurotoxin mu-SLPTX-Ssm6a (Ssam6). In some embodiments, one or more regions of the therapeutic peptide comprises an amino acid sequence based on or derived from an amino acid sequence of kappa-theraphotoxin-Tb1a (550). In some embodiments, one or more regions of the therapeutic peptide comprises an amino acid sequence based on or derived from an amino acid sequence of mambalign-1.

In some embodiments, one or more regions of the therapeutic peptide is configured to treat heart failure and/or fibrosis. In some embodiments, one or more regions of the therapeutic peptide is configured to treat heart failure and/or fibrosis related conditions. In some embodiments, 2, 3, 4, 5 or more regions of the therapeutic peptide are configured to treat heart failure and/or fibrosis. In some embodiments, 2, 3, 4, 5 or more regions of the therapeutic peptide are configured to treat heart failure and/or fibrosis related conditions. Heart failure related conditions may include coronary heart disease, high blood pressure, diabetes, cardiomyopathy, heart valve disease, arrhythmias, congenital heart defects, obstructive sleep apnea, myocarditis, hyperthyroidism, hypothyroidism, emphysema, hemochromatosis, and amyloidosis. Heart failure may be left-sided heart failure, right-sided heart failure, systolic heart failure, and diastolic heart failure. Fibrosis may include, but is not limited to, pulmonary fibrosis, idiopathic pulmonary fibrosis, cystic fibrosis, cirrhosis, endomyocardial fibrosis, myocardial infarction, atrial fibrosis, mediastinal fibrosis, myelofibrosis, retroperitoneal fibrosis, progressive massive fibrosis, nephrogenic systemic fibrosis, Crohn's disease, keloid, scleroderma/systemic sclerosis, arthrofibrosis, Peyronie's disease, Dupuytren's contracture, and adhesive capsulitis.

In some embodiments, one or more regions of the therapeutic peptide comprises an amino acid sequence based on or derived from an amino acid sequence which belongs to the insulin superfamily. In some embodiments, one or more regions of the therapeutic peptide comprises an amino acid sequence based on or derived from an amino acid sequence of insulin.

In some embodiments, amino acids of the therapeutic peptide, in whole or in part, are based on or derived from any one of SEQ ID NOs: 75-94, 223-229. The therapeutic peptide may comprise an amino acid sequence that is at least about 50% homologous to any one of SEQ ID NOs: 75-94, 223-229. The therapeutic peptide may comprise an amino acid sequence that is at least about 60%, 65%, 70%, 75%, 80%, 85%, 90%, 95%, or 97% homologous to any one of SEQ ID NOs: 75-94, 223-229. The therapeutic peptide may comprise an amino acid sequence that is at least about 70% homologous to any one of SEQ ID NOs: 75-94, 223-229. The therapeutic peptide may comprise an amino acid sequence that is at least about 80% homologous to any one of SEQ ID NOs: 75-94, 223-229. The therapeutic peptide may comprise an amino acid sequence that is at least about 50% identical to any one of SEQ ID NOs: 75-94, 223-229. The therapeutic peptide may comprise an amino acid sequence that is at least about 60%, 65%, 70%, 75%, 80%, 85%, 90%, 95%, or 97% identical to any one of SEQ ID NOs: 75-94, 223-229. The therapeutic peptide may comprise an amino acid sequence that is at least about 70% identical to any one of SEQ ID NOs: 75-94, 223-229. The therapeutic peptide may comprise an amino acid sequence that is at least about 80% identical to any one of SEQ ID NOs: 75-94, 223-229. The therapeutic peptide may comprise an amino acid sequence that is 100% identical to any one of SEQ ID NOs: 75-94, 223-229. In some embodiments, the therapeutic peptide comprises an amino acid sequence that is at least about 50%, 55%, 60%, 65%, 70%, 75%, 80%, 85%, 90%, 95%, or 97% homologous to an amino acid sequence of any one of SEQ ID NOs: 75-94, 223-229. In some embodiments, the therapeutic peptide comprises an amino acid sequence that is at least about 50%, 55%, 60%, 65%, 70%, 75%, 80%, 85%, 90%, 95%, or 97% identical to an amino acid sequence of any one of SEQ ID NOs: 75-94, 223-229. In some embodiments, the therapeutic peptide comprises an amino acid sequence that is 100% identical to an amino acid sequence of any one of SEQ ID NOs: 75-94, 223-229.

The therapeutic peptide may comprise an amino acid sequence comprising 10, 20, 30, 40, 50, 60, 70, 80, 90, 100 or more amino acids based on or derived from any one of SEQ ID NOs: 75-94, 223-229. The therapeutic peptide may comprise an amino acid sequence comprising 125, 150, 175, 200, 225, 250, 275, 300, 325, 350, 375, 400, 425, 450, 475, 450, 500 or more amino acids based on or derived from any one of SEQ ID NOs: 75-94, 223-229. The therapeutic peptide may comprise an amino acid sequence comprising 10 or more amino acids based on or derived from any one of SEQ ID NOs: 75-94, 223-229. The therapeutic peptide may comprise an amino acid sequence comprising 50 or more amino acids based on or derived from any one of SEQ ID NOs: 75-94, 223-229. The therapeutic peptide may comprise an amino acid sequence comprising 100 or more amino acids based on or derived from any one of SEQ ID NOs: 75-94, 223-229. The therapeutic peptide may comprise an amino acid sequence comprising 200 or more amino acids based on or derived from any one of SEQ ID NOs: 75-94, 223-229. The amino acids may be consecutive. Alternatively, or additionally, the amino acids are nonconsecutive. In some embodiments, the therapeutic peptide may comprise amino acids derived from any one of SEQ ID NOs: 75-94, 223-229 and amino acids not derived from any one of SEQ ID NOs: 75-94, 223-229. In some embodiments, the therapeutic peptide may comprise amino acids derived from one or more of SEQ ID NOs: 75-94, 223-229 and amino acids not derived from any one of SEQ ID NOs: 75-94, 223-229. In some embodiments, the therapeutic peptide comprises amino acids derived from 1, 2, 3, or 4 of SEQ ID NOs: 75-94, 223-229.

The therapeutic peptide may comprise a protease cleavage site. The protease cleavage site may be inserted within the therapeutic peptide. In some embodiments, the therapeutic peptide comprises a first therapeutic peptide region and a second therapeutic peptide region. In some embodiments, the therapeutic peptide comprises a protease cleavage site disposed between the first therapeutic peptide region and the second therapeutic peptide region. In some embodiments, the first therapeutic peptide region and the second therapeutic peptide region are derived from the same protein or set of amino acid sequences. In some embodiments, the first therapeutic peptide region and the second therapeutic peptide regions are derived from different proteins or sets of amino acid sequences. The one or more protease cleavage sites may be attached to the N-terminus, C-terminus or both the N- and C-termini of a region of a therapeutic peptide.

The therapeutic peptide may comprise one or more linker peptides. The therapeutic peptide may comprise two or more linker peptides. The therapeutic peptide may comprise 3, 4, 5, 6, 7 or more linker peptides. The linker peptides may be different. The linker peptides may be the same. The linker peptide may be inserted within the therapeutic peptide. In some embodiments, the therapeutic peptide comprises a first therapeutic region, a second therapeutic region, an one or more linker peptides positioned between the first therapeutic region and the second therapeutic region. The one or more linker peptides may be attached to the N-terminus, C-terminus or both the N- and C-termini of a region of a therapeutic peptide. In some embodiments, the linker peptide is derived from amino acids of any of SEQ ID NOs: 121-122.

The therapeutic peptide may comprise one or more internal linker. The internal linker may be inserted within the therapeutic peptide. In some embodiments, the therapeutic peptide comprises a first therapeutic peptide region and a second therapeutic peptide region. In some embodiments, the therapeutic peptide comprises a internal linker disposed between the first therapeutic peptide region and the second therapeutic peptide region. In some embodiments, the first therapeutic peptide region and the second therapeutic peptide region are derived from the same protein or set of amino acid sequences. In some embodiments, the first therapeutic peptide region and the second therapeutic peptide regions are derived from different proteins or sets of amino acid sequences. In some embodiments, the internal linker is derived from amino acids of any of SEQ ID NOs: 123-126, 240-244. In some embodiments, the internal linker comprises amino acids having repeating sequences. In some embodiments, the internal linker has 2, 3, 4, 5, 6, 7, 8, 9, 10 or more repeating sequences. In some embodiments, the internal linker is low immunogenic. In some embodiments, the internal linker is biodegradable.

Non-Immunoglobulin Region

The immunoglobulin fusion proteins disclosed herein may comprise one or more non-immunoglobulin regions. The immunoglobulin fusion proteins disclosed herein may comprise two or more non-immunoglobulin regions. The immunoglobulin fusion proteins disclosed herein may comprise 3, 4, 5, 6, 7, 8, 9, 10 or more non-immunoglobulin regions. In some embodiments, a non-immunoglobulin region is a region which is not based on or derived from an immunoglobulin region disclosed herein. In one embodiment, the non-immunoglobulin region does not comprise amino acids based on or derived from an immunoglobulin region disclosed herein or provided herein in any SEQ ID. In one embodiment, a non-immunoglobulin region does not comprise more than 10, 11, 12, 13, 14, 15, 16, 17, 18, 19, 20, 25, 30, 35, 40, 45, 50, 55, 60, 70, 80, 90, 100, 150, 200, 400, 500, or more amino acids based on or derived from an immunoglobulin region.

The two or more non-immunoglobulin regions may be attached to one or more immunoglobulin regions. The two or more non-immunoglobulin regions may be attached to two or more immunoglobulin regions. The two or more non-immunoglobulin regions may be attached to one or more immunoglobulin chains. The two or more non-immunoglobulin regions may be attached to two or more immunoglobulin chains. The two or more non-immunoglobulin regions may be attached to one or more units within the one or more immunoglobulin regions. The two or more non-immunoglobulin regions may be attached to two or more units within the one or more immunoglobulin regions.

The non-immunoglobulin regions may comprise one or more therapeutic peptides. The non-immunoglobulin regions may comprise two or more therapeutic peptides. The non-immunoglobulin regions may comprise 3, 4, 5, 6, 7 or more therapeutic peptides. The therapeutic peptides may be different. The therapeutic peptides may be the same. In some embodiments, the therapeutic peptide is derived from amino acids of any of SEQ ID NOs: 75-94, 223-229. The therapeutic peptide may comprise one or more internal linker. The internal linker may be inserted within the therapeutic peptide. In some embodiments, the therapeutic peptide comprises a first therapeutic peptide region and a second therapeutic peptide region. In some embodiments, the therapeutic peptide comprises a internal linker disposed between the first therapeutic peptide region and the second therapeutic peptide region. In some embodiments, the first therapeutic peptide region and the second therapeutic peptide region are derived from the same protein or set of amino acid sequences. In some embodiments, the first therapeutic peptide region and the second therapeutic peptide regions are derived from different proteins or sets of amino acid sequences. In some embodiments, the internal linker is derived from amino acids of any of SEQ ID NOs: 123-126, 240-244.

The non-immunoglobulin regions may comprise one or more extender peptides. The non-immunoglobulin regions may comprise two or more extender peptides. The non-immunoglobulin regions may comprise 3, 4, 5, 6, 7 or more extender peptides. The extender peptides may be different. The extender peptides may be the same. The non-immunoglobulin region comprising one or more extender peptides may be referred to as an extender fusion region. In some embodiments, the extender peptide is derived from amino acids of any of SEQ ID NOs: 119-120. In some embodiments, the one or more extender peptides is attached to the N-terminus, C-terminus or both the N- and C-termini of an immunoglobulin region. In some embodiments, the one or more extender peptides is attached to the N-terminus, C-terminus or both the N- and C-termini of a therapeutic peptide region.

The non-immunoglobulin region may comprise a protease cleavage site. The non-immunoglobulin regions may comprise two or more protease cleavage sites. The non-immunoglobulin regions may comprise 3, 4, 5, 6, 7 or more protease cleavage sites. The protease cleavage sites may be different. The protease cleavage sites may be the same. In some embodiments, the one or more protease cleavage sites is attached to the N-terminus, C-terminus or both the N- and C-termini of an immunoglobulin region. In some embodiments, the one or more protease cleavage sites is attached to the N-terminus, C-terminus or both the N- and C-termini of a therapeutic peptide region.

The non-immunoglobulin region may comprise a linker peptide. The non-immunoglobulin regions may comprise two or more linker peptides. The non-immunoglobulin regions may comprise 3, 4, 5, 6, 7 or more linker peptides. The linker peptides may be different. The linker peptides may be the same. In some embodiments, the linker peptide is derived from amino acids of any of SEQ ID NOs: 121-122. In some embodiments, the one or more linker peptides is attached to the N-terminus, C-terminus or both the N- and C-termini of an immunoglobulin region. In some embodiments, the one or more linker peptides is attached to the N-terminus, C-terminus or both the N- and C-termini of a therapeutic peptide region. In some embodiments, the one or more linker peptides is attached to the N-terminus, C-terminus or both the N- and C-termini of an extender peptide.

The non-immunoglobulin region may be inserted into the immunoglobulin region. Insertion of the non-immunoglobulin region into the immunoglobulin region may comprise removal or deletion of a portion of the immunoglobulin from which the immunoglobulin region is based on or derived from. The non-immunoglobulin region may replace at least a portion of a heavy chain. The non-immunoglobulin region may replace at least a portion of a light chain. The non-immunoglobulin region may replace at least a portion of a V region. The non-immunoglobulin region may replace at least a portion of a D region. The non-immunoglobulin region may replace at least a portion of a J region. The non-immunoglobulin region may replace at least a portion of a variable region. The non-immunoglobulin region may replace at least a portion of a constant region. The non-immunoglobulin region may replace at least a portion of a complementarity determining region (CDR). The non-immunoglobulin region may replace at least a portion of a CDR1. The non-immunoglobulin region may replace at least a portion of a CDR2. The non-immunoglobulin region may replace at least a portion of a CDR3. The non-immunoglobulin region may replace at least about 5%, 10%, 15%, 20%, 25%, 30%, 35%, 40%, 45%, 50%, 55%, 60%, 65%, 70%, 75%, 80%, 85%, 90%, 95%, or more of the immunoglobulin or portion thereof. For example, the non-immunoglobulin region may replace at least about 50% of a CDR. The non-immunoglobulin region may replace at least about 70% of a CDR. The non-immunoglobulin region may replace at least about 80% of a CDR. The non-immunoglobulin region may replace at least about 90% of a CDR. The non-immunoglobulin region may replace at least about 95% of a CDR.

In some embodiments, the one or more non-immunoglobulin regions of the immunoglobulin fusion protein comprises an amino acid sequence based on or derived from an amino acid sequence of leptin. In some embodiments, a therapeutic peptide of the non-immunoglobulin region of the immunoglobulin fusion protein comprises an amino acid sequence based on or derived from an amino acid sequence of leptin.

In some embodiments, amino acids of the non-immunoglobulin region, in whole or in part, are based on or derived from any one of SEQ ID NOs: 144-160, 255-264. The non-immunoglobulin region may comprise an amino acid sequence that is at least about 50% homologous to any one of SEQ ID NOs: 144-160, 255-264. The non-immunoglobulin region may comprise an amino acid sequence that is at least about 60%, 65%, 70%, 75%, 80%, 85%, 90%, 95%, or 97% homologous to any one of SEQ ID NOs: 144-160, 255-264. The non-immunoglobulin region may comprise an amino acid sequence that is at least about 70% homologous to any one of SEQ ID NOs: 144-160, 255-264. The non-immunoglobulin region may comprise an amino acid sequence that is at least about 80% homologous to any one of SEQ ID NOs: 144-160, 255-264. The non-immunoglobulin region may comprise an amino acid sequence that is at least about 50% identical to any one of SEQ ID NOs: 144-160, 255-264. The non-immunoglobulin region may comprise an amino acid sequence that is at least about 60%, 65%, 70%, 75%, 80%, 85%, 90%, 95%, or 97% identical to any one of SEQ ID NOs: 144-160, 255-264. The non-immunoglobulin region may comprise an amino acid sequence that is at least about 70% identical to any one of SEQ ID NOs: 144-160, 255-264. The non-immunoglobulin region may comprise an amino acid sequence that is at least about 80% identical to any one of SEQ ID NOs: 144-160, 255-264. The non-immunoglobulin region may comprise an amino acid sequence that is 100% identical to any one of SEQ ID NOs: 144-160, 255-264. In some embodiments, the non-immunoglobulin region comprises an amino acid sequence that is at least about 50%, 55%, 60%, 65%, 70%, 75%, 80%, 85%, 90%, 95%, or 97% homologous to an amino acid sequence of any one of SEQ ID NOs: 144-160, 255-264. In some embodiments, the non-immunoglobulin region comprises an amino acid sequence that is at least about 50%, 55%, 60%, 65%, 70%, 75%, 80%, 85%, 90%, 95%, or 97% identical to an amino acid sequence of any one of SEQ ID NOs: 144-160, 255-264. In some embodiments, the non-immunoglobulin region comprises an amino acid sequence that is 100% identical to an amino acid sequence of any one of SEQ ID NOs: 144-160, 255-264.

The non-immunoglobulin region may comprise an amino acid sequence comprising 10, 20, 30, 40, 50, 60, 70, 80, 90, 100 or more amino acids based on or derived from any one of SEQ ID NOs: 144-160, 255-264. The non-immunoglobulin region may comprise an amino acid sequence comprising 125, 150, 175, 200, 225, 250, 275, 300, 325, 350, 375, 400, 425, 450, 475, 450, 500 or more amino acids based on or derived from any one of SEQ ID NOs: 144-160, 255-264. The non-immunoglobulin region may comprise an amino acid sequence comprising 10 or more amino acids based on or derived from any one of SEQ ID NOs: 144-160, 255-264. The non-immunoglobulin region may comprise an amino acid sequence comprising 50 or more amino acids based on or derived from any one of SEQ ID NOs: 144-160, 255-264. The non-immunoglobulin region may comprise an amino acid sequence comprising 100 or more amino acids based on or derived from any one of SEQ ID NOs: 144-160, 255-264. The non-immunoglobulin region may comprise an amino acid sequence comprising 200 or more amino acids based on or derived from any one of SEQ ID NOs: 144-160, 255-264. The amino acids may be consecutive. Alternatively, or additionally, the amino acids are nonconsecutive. In some embodiments, the non-immunoglobulin region may comprise amino acids derived from any one of SEQ ID NOs: 144-160, 255-264 and amino acids not derived from any one of SEQ ID NOs: 144-160, 255-264. In some embodiments, the non-immunoglobulin region may comprise amino acids derived from one or more of SEQ ID NOs: 144-160, 255-264 and amino acids not derived from any one of SEQ ID NOs: 144-160, 255-264. In some embodiments, the non-immunoglobulin region comprises amino acids derived from 1, 2, 3, or 4 of SEQ ID NOs: 144-160, 255-264.

### Extender Peptide

The immunoglobulin fusion proteins disclosed herein may comprise one or more extender peptides. The one or more extender peptides may be attached to the N-terminus, C-terminus, or N- and C-terminus of a therapeutic peptide. The one or more extender peptides may be attached to each end of a therapeutic peptide. The one or more extender peptides may be attached to different ends of a therapeutic peptide. The one or more extender peptides may be attached to the N-terminus, C-terminus, or N- and C-terminus of a linker, wherein the linker is attached to a therapeutic peptide. The one or more extender peptides may be attached to the N-terminus, C-terminus, or N- and C-terminus of an immunoglobulin region. The one or more extender peptides may be attached to each end of an immunoglobulin region. The one or more extender peptides may be attached to different ends of an immunoglobulin region.

The extender fusion region of the immunoglobulin fusion proteins disclosed herein may comprise one or more extender peptides. The extender fusion region may comprise 2 or more extender peptides. The extender fusion region may comprise 3 or more extender peptides. The extender fusion region may comprise 4 or more extender peptides. The extender fusion region may comprise 5 or more extender peptides. The extender fusion region may comprise a first extender peptide and a second extender peptide.

The extender peptide may comprise one or more secondary structures. The extender peptide may comprise two or more secondary structures. The extender peptide may comprise 3, 4, 5, 6, 7 or more secondary structures. The two or more extender peptide may comprise one or more secondary structures. The two or more extender peptides may comprise two or more secondary structures. The two or more extender peptides may comprise 3, 4, 5, 6, 7 or more secondary structures. Each extender peptide may comprise at least one secondary structure. The secondary structures of the two or more extender peptides may be the same. Alternatively, the secondary structures of the two or more extender peptides may be different. In some embodiments, the extender peptide does not comprise a regular secondary structure.

The one or more secondary structures may comprise one or more beta strands. The extender peptides may comprise two or more beta strands. For example, the first extender peptide comprises a first beta strand and the second extender peptide comprises a second beta strand. The extender peptides may comprise 3, 4, 5, 6, 7 or more beta strands. The two or more beta strands may be anti-parallel. The two or more beta strands may be parallel.

Alternatively, or additionally, the one or more secondary structures may comprise one or more alpha helices. The extender peptides may comprise two or more alpha helices. For example, the first extender peptide comprises a first alpha helix and the second extender peptide comprises a second alpha helix. The extender peptides may comprise 3, 4, 5, 6, 7 or more alpha helices. The two or more alpha helices may be anti-parallel. The two or more alpha helices may be parallel. The two or more alpha helices may form one or more coiled-coil domains.

The one or more extender peptides may comprise at least about 1, 2, 3, 4, 5, 6, 7, 8, 9, 10 or more amino acids. The one or more extender peptides may comprise at least about 10, 11, 12, 13, 14, 15, 16, 17, 18, 19, 20, 21, 22, 23, 24, 25, 26, 27, 28, 29, or 30 or more amino acids. The one or more extender peptides may comprise at least about 35, 40, 45, 50 or more amino acids.

The one or more extender peptides may comprise less than about 100 amino acids. The one or more extender peptides may comprise less than about 95, 90, 85, 80, 75, 70, 65, 60, 55, or 50 amino acids. The one or more extender peptides may comprise less than about 90 amino acids. The one or more extender peptides may comprise less than about 80 amino acids. The one or more extender peptides may comprise less than about 70 amino acids.

The two or more extender peptides may be the same length. For example, the first extender peptide and the second extender peptide are the same length. Alternatively, the two or more extender peptides are different lengths. In another example, the first extender peptide and the second extender peptide are different lengths. The two or more extender peptides may differ in length by at least about 1, 2, 3, 4, 5, 6, 7, 8, 9, 10 or more amino acids. The two or more extender peptides may differ in length by at least about 1 or more amino acids. The two or more extender peptides may differ in length by at least about 3 or more amino acids. The two or more extender peptides may differ in length by at least about 5 or more amino acids.

The extender peptide may be adjacent to an immunoglobulin region. The extender peptide may be attached to the N-terminus, C-terminus, or N- and C-terminus of the immunoglobulin region. The extender peptide may be adjacent to a non-immunoglobulin region. The extender peptide may be attached to the N-terminus, C-terminus, or N- and C-terminus of the non-immunoglobulin region. The extender peptide may be adjacent to a therapeutic peptide. The extender peptide may be attached to the N-terminus, C-terminus, or N- and C-terminus of the therapeutic peptide. The extender peptide may be adjacent to a linker. The extender peptide may be attached to the N-terminus, C-terminus, or N- and C-terminus of the linker. The extender peptide may be adjacent to a proteolytic cleavage site. The extender peptide may be attached to the N-terminus, C-terminus, or N- and C-terminus of the proteolytic cleavage site.

The extender peptide may connect the therapeutic peptide to the immunoglobulin region. The extender peptide may be positioned between the immunoglobulin region and the therapeutic peptide, linker, and/or proteolytic cleavage site. The extender peptide may be between two or more immunoglobulin regions, therapeutic peptides, linkers, proteolytic cleavage sites or a combination thereof. The extender peptide may be N-terminal to the immunoglobulin region, therapeutic peptide, the linker, the proteolytic cleavage site, or a combination thereof. The extender peptide may be C-terminal to the immunoglobulin region, therapeutic peptide, the linker, the proteolytic cleavage site, or a combination thereof.

The extender peptide may comprise an amino acid sequence that is based on or derived from any one of SEQ ID NOs: 119-120. The extender peptide may comprise an amino acid sequence that is at least about 50% homologous to an amino acid sequence based on or derived from any one of SEQ ID NOs: 119-120. The extender peptide may comprise an amino acid sequence that is at least about or more homologous to an amino acid sequence based on or derived from any one of SEQ ID NOs: 119-120. The extender peptide may comprise an amino acid sequence that is at least about 70% homologous to an amino acid sequence based on or derived from any one of SEQ ID NOs: 119-120. The extender peptide may comprise an amino acid sequence that is at least about 80% homologous to an amino acid sequence based on or derived from any one of SEQ ID NOs: 119-120. The extender peptide may comprise an amino acid sequence that is at least about 85% homologous to an amino acid sequence based on or derived from any one of SEQ ID NOs: 119-120.

The first extender peptide may comprise an amino acid sequence that is based on or derived from any one of SEQ ID NOs: 119-120. The first extender peptide may comprise an amino acid sequence that is at least about 50% homologous to an amino acid sequence based on or derived from any one of SEQ ID NOs: 119-120. The first extender peptide may comprise an amino acid sequence that is at least about 60%, 65%, 70%, 75%, 80%, 85%, 90%, 95%, 97% or more homologous to an amino acid sequence based on or derived from any one of SEQ ID NOs: 119-120. The first extender peptide may comprise an amino acid sequence that is at least about 75% homologous to an amino acid sequence based on or derived from any one of SEQ ID NOs: 119-120. The first extender peptide may comprise an amino acid sequence that is at least about 80% homologous to an amino acid sequence based on or derived from any one of SEQ ID NOs: 119-120.

The second extender peptide may comprise an amino acid sequence that is based on or derived from any one of SEQ ID NOs: 119-120. The second extender peptide may comprise an amino acid sequence that is at least about 50% homologous to an amino acid sequence based on or derived from any one of SEQ ID NOs: 119-120. The second extender peptide may comprise an amino acid sequence that is at least about 60%, 65%, 70%, 75%, 80%, 85%, 90%, 95%, 97% or more homologous to an amino acid sequence based on or derived from any one of SEQ ID NOs: 119-120. The second extender peptide may comprise an amino acid sequence that is at least about 70% homologous to an amino acid sequence based on or derived from any one of SEQ ID NOs: 119-120. The second extender peptide may comprise an amino acid sequence that is at least about 80% homologous to an amino acid sequence based on or derived from any one of SEQ ID NOs: 119-120.

The immunoglobulin fusion protein may comprise (a) a first extender peptide comprising an amino acid sequence based on or derived from SEQ ID NO: 119; and (b) a second extender peptide comprising an amino acid sequence based on or derived from SEQ ID NO: 120. The immunoglobulin fusion protein may comprise (a) a first extender peptide comprising an amino acid sequence that is at least about 50% homologous to an amino acid sequence of SEQ ID NO: 119; and (b) a second extender peptide comprising an amino acid sequence that is at least about 50% homologous to an amino acid sequence of SEQ ID NO: 120. The first extender peptide may comprise an amino acid sequence that is at least about 60%, 65%, 70%, 75%, 80%, 85%, 90%, 95% or more homologous to an amino acid sequence of SEQ ID NO: 119. The second extender peptide may comprise an amino acid sequence that is at least about 60%, 65%, 70%, 75%, 80%, 85%, 90%, 95% or more homologous to an amino acid sequence of SEQ ID NO: 120. The first extender peptide may comprise an amino acid sequence comprising 3, 4, 5, 6, 7 or more amino acids based on or derived from an amino acid sequence of SEQ ID NO: 119. The first extender peptide may comprise an amino acid sequence comprising 5 or more amino acids based on or derived from an amino acid sequence of SEQ ID NO: 119. The second extender peptide may comprise an amino acid sequence comprising 3, 4, 5, 6, 7 or more amino acids based on or derived from an amino acid sequence of SEQ ID NO: 120. The second extender peptide may comprise an amino acid sequence comprising 5 or more amino acids based on or derived from an amino acid sequence of SEQ ID NO: 120.

The extender peptides disclosed herein may be based on or derived from a CDR3. The CDR3 may be an ultralong CDR3. An “ultralong CDR3” or an “ultralong CDR3 sequence”, used interchangeably herein, may comprise a CDR3 that is not derived from a human immunoglobulin sequence. An ultralong CDR3 may be 35 amino acids in length or longer, for example, 40 amino acids in length or longer, 45 amino acids in length or longer, 50 amino acids in length or longer, 55 amino acids in length or longer, or 60 amino acids in length or longer. The ultralong CDR3 may be a heavy chain CDR3 (CDR-H3 or CDRH3). The ultralong CDR3 may comprise a sequence derived from or based on a ruminant (e.g., bovine) sequence. An ultralong CDR3 may comprise one or more cysteine motifs. An ultralong CDR3 may comprise at least 3 or more cysteine residues, for example, 4 or more cysteine residues, 6 or more cysteine residues, or 8 or more cysteine residues. Additional details on ultralong CDR3 sequences can be found in Saini S S, et al. (Exceptionally long CDR3H region with multiple cysteine residues in functional bovine IgM antibodies, European Journal of Immunology, 1999), Zhang Y, et al. (Functional immunoglobulin CDR3 fusion proteins with enhanced pharmacological properties, Angew Chem Int Ed Engl, 2013), Wang F, et al. (Reshaping immunoglobulin diversity, Cell, 2013) and U.S. Pat. No. 6,740,747.

The extender peptides may comprise 7 or fewer amino acids based on or derived from a CDR. The extender peptides may comprise 6, 5, 4, 3, 2, 1 or fewer amino acids based on or derived from a CDR. The amino acids may be consecutive. The amino acids may be non-consecutive. The CDR may be CDR1. The CDR may be CDR2. The CDR may be CDR3. The CDR may be an ultralong CDR.

The extender peptides may be based on or derived from a CDR, wherein the CDR is not an ultralong CDR3. The extender peptides may comprise 10 or fewer amino acids based on or derived from a CDR3. The extender peptides may comprise 9, 8, 7, 6, 5, 4, 3, 2, 1 or fewer amino acids based on or derived from a CDR3. The extender peptides may comprise 8 or fewer amino acids based on or derived from a CDR3. The extender peptides may comprise 7 or fewer amino acids based on or derived from a CDR3. The extender peptides may comprise 5 or fewer amino acids based on or derived from a CDR3.

The extender peptides may comprise an amino acid sequence that is less than about 50% identical to an amino acid sequence comprising an ultralong CDR3. The extender peptides may comprise an amino acid sequence that is less than about 45%, 40%, 35%, 30%, 25%, 20%, 25%, or 10% identical to an amino acid sequence comprising an ultralong CDR3. The extender peptides may comprise an amino acid sequence that is less than about 30% identical to an amino acid sequence comprising an ultralong CDR3. The extender peptides may comprise an amino acid sequence that is less than about 25% identical to an amino acid sequence comprising an ultralong CDR3. The extender peptides may comprise an amino acid sequence that is less than about 20% identical to an amino acid sequence comprising an ultralong CDR3.

The extender peptide may comprise 1, 2, 3, 4, 5, 6, 7, 8, 9, 10, 11, 12, 13, 14, 15 or more amino acids attached to or inserted into an ultralong CDR3-based portion of the extender peptide. The extender peptide may comprise 1 or more amino acids attached to or inserted into an ultralong CDR3-based portion of the extender peptide. The extender peptide may comprise 3 or more amino acids attached to or inserted into an ultralong CDR3-based portion of the extender peptide. The extender peptide may comprise 5 or more amino acids attached to or inserted into an ultralong CDR3-based portion of the extender peptide. The two or more amino acids attached to or inserted into the ultralong CDR3 may be contiguous. Alternatively, or additionally, the two or more amino acids attached to or inserted into the ultralong CDR3 are not contiguous.

The extender peptide may comprise 30, 25, 20, 19, 18, 17, 16, 15, 14, 13, 12, 11, 10 or fewer amino acids attached to or inserted into an ultralong CDR3-based portion of the extender peptide. The extender peptide may comprise 20 or fewer amino acids attached to or inserted into an ultralong CDR3-based portion of the extender peptide. The extender peptide may comprise 15 or fewer amino acids attached to or inserted into an ultralong CDR3-based portion of the extender peptide. The extender peptide may comprise 10 or fewer amino acids attached to or inserted into an ultralong CDR3-based portion of the extender peptide. The amino acids attached to or inserted into the ultralong CDR3 may be contiguous. Alternatively, or additionally, the amino acids attached to or inserted into the ultralong CDR3 are not contiguous.

The aliphatic amino acids may comprise at least about 20% of the total amino acids of the extender peptides. The aliphatic amino acids may comprise at least about 22%, 25%, 27%, 30%, 32%, 35%, 37%, 40%, 42%, 45% or more of the total amino acids of the extender peptides. The aliphatic amino acids may comprise at least about 22% of the total amino acids of the extender peptides. The aliphatic amino acids may comprise at least about 27% of the total amino acids of the extender peptides.

The aliphatic amino acids may comprise less than about 50% of the total amino acids of the extender peptides. The aliphatic amino acids may comprise less than about 47%, 45%, 43%, 40%, 38%, 35%, 33% or 30% of the total amino acids of the extender peptides.

The aliphatic amino acids may comprise between about 20% to about 45% of the total amino acids of the extender peptides. The aliphatic amino acids may comprise between about 23% to about 45% of the total amino acids of the extender peptides. The aliphatic amino acids may comprise between about 23% to about 40% of the total amino acids of the extender peptides.

The aromatic amino acids may comprise less than about 20% of the total amino acids of the extender peptides. The aromatic amino acids may comprise less than about 19%, 18%, 17%, 16%, 15%, 14%, 13%, 12%, 11% or 10% of the total amino acids of the extender peptides. The aromatic amino acids may comprise between 0% to about 20% of the total amino acids of the extender peptides.

The non-polar amino acids may comprise at least about 30% of the total amino acids of the extender peptides. The non-polar amino acids may comprise at least about 31%, 32%, 33%, 34%, 35%, 36%, 37%, 38%, 39%, or 40% of the total amino acids of the extender peptides. The non-polar amino acids may comprise at least about 32% of the total amino acids of the extender peptides.

The non-polar amino acids may comprise less than about 80% of the total amino acids of the extender peptides. The non-polar amino acids may comprise less than about 77%, 75%, 72%, 70%, 69%, or 68% of the total amino acids of the extender peptides.

The non-polar amino acids may comprise between about 35% to about 80% of the total amino acids of the extender peptides. The non-polar amino acids may comprise between about 38% to about 80% of the total amino acids of the extender peptides. The non-polar amino acids may comprise between about 38% to about 75% of the total amino acids of the extender peptides. The non-polar amino acids may comprise between about 35% to about 70% of the total amino acids of the extender peptides.

The polar amino acids may comprise at least about 20% of the total amino acids of the extender peptides. The polar amino acids may comprise at least about 22%, 23%, 24%, 25%, 26%, 27%, 28%, 29%, 30%, 35% or more of the total amino acids of the extender peptides. The polar amino acids may comprise at least about 23% of the total amino acids of the extender peptides.

The polar amino acids may comprise less than about 80% of the total amino acids of the extender peptides. The polar amino acids may comprise less than about 77%, 75%, 72%, 70%, 69%, or 68% of the total amino acids of the extender peptides. The polar amino acids may comprise less than about 77% of the total amino acids of the extender peptides. The polar amino acids may comprise less than about 75% of the total amino acids of the extender peptides. The polar amino acids may comprise less than about 72% of the total amino acids of the extender peptides.

The polar amino acids may comprise between about 25% to about 70% of the total amino acids of the extender peptides. The polar amino acids may comprise between about 27% to about 70% of the total amino acids of the extender peptides. The polar amino acids may comprise between about 30% to about 70% of the total amino acids of the extender peptides.

Alternatively, the immunoglobulin fusion proteins disclosed herein do not comprise an extender peptide.

### Linkers

The immunoglobulin fusion proteins, immunoglobulin regions, therapeutic peptides, non-immunoglobulin regions and/or extender fusion regions may further comprise one or more linkers. The immunoglobulin fusion proteins, immunoglobulin regions, non-immunoglobulin regions and/or extender fusion regions may further comprise 2, 3, 4, 5, 6, 7, 8, 9, 10 or more linkers. The extender fusion region may further comprise one or more linkers. The extender fusion region may further comprise 2, 3, 4, 5, 6, 7, 8, 9, 10 or more linkers.

The one or more linkers are attached to the N-terminus, C-terminus or both N- and C-termini of a therapeutic peptide. The one or more linkers are attached to the N-terminus, C-terminus or both N- and C-termini of the extender peptide. The one or more linkers are attached to the N-terminus, C-terminus or both N- and C-termini of a proteolytic cleavage site. The one or more linkers may be attached to a therapeutic peptide, extender peptide, proteolytic cleavage site, extender fusion region, immunoglobulin region, non-immunoglobulin region or a combination thereof.

The one or more linkers may comprise an amino acid sequence selected from any one of SEQ ID NOs:121-122. The one or more linkers may comprise an amino acid sequence that is at least about 50% homologous to any one of SEQ ID NOs: 121-122. The one or more linkers may comprise an amino acid sequence that is at least about 60%, 65%, 70%, 75%, 80%, 85%, 90%, 95% or more homologous to any one of SEQ ID NOs: 121-122. The one or more linkers may comprise an amino acid sequence that is at least about 70% homologous to any one of SEQ ID NOs: 121-122. The one or more linkers may comprise an amino acid sequence that is at least about 80% homologous to any one of SEQ ID NOs: 121-122.

In some embodiments, the linker is a connecting linker. The connecting linker may link the therapeutic peptide to an immunoglobulin region. The connecting linker may comprise an amino acid sequence that is at least about 50% homologous to any of SEQ ID NOs: 115-118, 237-239. The connecting linker may comprise an amino acid sequence that is at least about 60%, 65%, 70%, 75%, 80%, 85%, 90%, 95% or more homologous to any one of SEQ ID NOs: 115-118, 237-239. The connecting linker may comprise an amino acid sequence that is at least about 70% homologous to any one of SEQ ID NOs: 115-118, 237-239. The connecting linker may comprise an amino acid sequence that is at least about 80% homologous to any one of SEQ ID NOs: 115-118, 237-239.

In some embodiments, the linker is an internal linker. The internal linker may be a portion of a therapeutic peptide. The internal linker may link two regions of a therapeutic peptide. The internal linker may link two therapeutic peptides derived from two different peptides or proteins. The internal linker may link two therapeutic peptides derived from the same peptide or protein. The internal linker may comprise an amino acid sequence that is at least about 50% homologous to any of SEQ ID NOs: 123-126, 240-244. The internal linker may comprise an amino acid sequence that is at least about 60%, 65%, 70%, 75%, 80%, 85%, 90%, 95% or more homologous to any one of SEQ ID NOs: 123-126, 240-244. The internal linker may comprise an amino acid sequence that is at least about 70% homologous to any one of SEQ ID NOs: 123-126, 240-244. The internal linker may comprise an amino acid sequence that is at least about 80% homologous to any one of SEQ ID NOs: 123-126, 240-244.

### Proteolytic Cleavage Site

The immunoglobulin fusion proteins disclosed herein may further comprise one or more proteolytic cleavage sites. The immunoglobulin fusion proteins disclosed herein may further comprise 2 or more proteolytic cleavage sites. The immunoglobulin fusion proteins disclosed herein may further comprise 3 or more proteolytic cleavage sites. The immunoglobulin fusion proteins disclosed herein may further comprise 4, 5, 6, 7 or more proteolytic cleavage sites. The therapeutic peptides disclosed herein may further comprise one or more proteolytic cleavage sites.

The one or more proteolytic cleavage sites may be attached to the N-terminus, C-terminus or both N- and C-termini of a therapeutic peptide. The one or more proteolytic cleavage sites may attached to the N-terminus, C-terminus or both N- and C-termini of the extender peptide. The one or more proteolytic cleavage sites may attached to the N-terminus, C-terminus or both N- and C-termini of a linker. The one or more proteolytic cleavage sites may be attached to a therapeutic peptide, extender peptide, linker, extender fusion region, immunoglobulin region, non-immunoglobulin region or a combination thereof.

In some embodiments, the proteolytic cleavage site is located within the amino acid sequence of the therapeutic peptide, extender peptide, immunoglobulin region, or a combination thereof. The therapeutic peptide may comprise one or more proteolytic cleavage sites within its amino acid sequence. For example, SEQ ID NOs: 99-101 disclose a relaxin protein comprising two internal proteolytic cleavage sites.

Two or more proteolytic cleavage sites may surround a therapeutic peptide, extender peptide, linker, immunoglobulin region, or combination thereof. Digestion of the proteolytic cleavage site may result in release of a peptide fragment located between the two or more proteolytic cleavage sites. For example, the proteolytic cleavage sites may flank a therapeutic peptide-linker peptide. Digestion of the proteolytic cleavage sites may result in release of the therapeutic peptide-linker.

The proteolytic cleavage site may be recognized by one or more proteases. The one or more proteases may be a serine protease, threonine protease, cysteine protease, aspartate protease, glutamic protease, metalloprotease, exopeptidases, endopeptidases, or a combination thereof. The proteases may be selected from the group comprising Factor VII or Factor Xa. Additional examples of proteases include, but are not limited to, aminopeptidases, carboxypeptidases, trypsin, chymotrypsin, pepsin, papain, and elastase. The protease may be PC2. In some embodiments, the protease recognizes the amino acid sequence KR. In some embodiments, the protease recognizes the amino acid sequence RKKR (SEQ ID NO: 267).

### Vectors, Host Cells and Recombinant Methods

Immunoglobulin fusion proteins, as disclosed herein, may be expressed and purified by known recombinant and protein purification methods. In some instances, the activity of the immunoglobulin fusion protein is affected by expression and/or purification methods. For example, the activity of an immunoglobulin fusion protein configured for use as a therapeutic, is enhanced or attenuated based on the identity of the expression vector, identity of the recombinant host, identity of the cell line, expression reaction conditions, purification methods, protein processing, or any combination thereof. Expression reaction conditions include, but are not limited to, temperature, % CO₂, media, expression time, cofactors, and chaperones. Purification methods include, but are not limited to, purification temperatures, chromatography resins, protease inhibitors, and buffer compositions.

Immunoglobulin fusion proteins, as disclosed herein, may be expressed by recombinant methods. Generally, a nucleic acid encoding an immunoglobulin fusion protein may be isolated and inserted into a replicable vector for further cloning (amplification of the DNA) or for expression. DNA encoding the immunoglobulin fusion protein may be prepared by PCR amplification and sequenced using conventional procedures (e.g., by using oligonucleotide probes that are capable of binding specifically to nucleotides encoding Immunoglobulin fusion proteins). In an exemplary embodiment, nucleic acid encoding an immunoglobulin fusion protein is PCR amplified, restriction enzyme digested and gel purified. The digested nucleic acid may be inserted into a replicable vector. The replicable vector containing the digested immunoglobulin fusion protein insertion may be transformed or transduced into a host cell for further cloning (amplification of the DNA) or for expression. Host cells may be prokaryotic or eukaryotic cells.

Polynucleotide sequences encoding polypeptide components (e.g., immunoglobulin region, extender peptide, therapeutic peptide) of the immunoglobulin fusion proteins may be obtained by PCR amplification. Polynucleotide sequences may be isolated and sequenced from cells containing nucleic acids encoding the polypeptide components. Alternatively, or additionally, polynucleotides may be synthesized using nucleotide synthesizer or PCR techniques. Once obtained, sequences encoding the polypeptide components may be inserted into a recombinant vector capable of replicating and expressing heterologous polynucleotides in prokaryotic and/or eukaryotic hosts.

In addition, phage vectors containing replicon and control sequences that are compatible with the host microorganism may be used as transforming vectors in connection with these hosts. For example, bacteriophage such as λGEM™-11 may be utilized in making a recombinant vector which may be used to transform susceptible host cells such as E. coli LE392.

Immunoglobulin fusion proteins may be expressed intracellularly (e.g., cytoplasm) or extracellularly (e.g., secretion). For extracellular expression, the vector may comprise a secretion signal which enables translocation of the immunoglobulin fusion proteins to the outside of the cell.

Suitable host cells for cloning or expression of immunoglobulin fusion proteins-encoding vectors include prokaryotic or eukaryotic cells. The host cell may be a eukaryotic. Examples of eukaryotic cells include, but are not limited to, Human Embryonic Kidney (HEK) cell, Chinese Hamster Ovary (CHO) cell, fungi, yeasts, invertebrate cells (e.g., plant cells and insect cells), lymphoid cell (e.g., YO, NSO, Sp20 cell). Other examples of suitable mammalian host cell lines are monkey kidney CV1 line transformed by SV40 (COS-7); baby hamster kidney cells (BHK); mouse sertoli cells; monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells; MRC 5 cells; and FS4 cells. The host cell may be a prokaryotic cell (e.g., E. coli).

Host cells may be transformed with vectors containing nucleotides encoding an immunoglobulin fusion proteins. Transformed host cells may be cultured in media. The media may be supplemented with one or more agents for inducing promoters, selecting transformants, or amplifying or expressing the genes encoding the desired sequences. Methods for transforming host cells are known in the art and may include electroporation, calcium chloride, or polyethylene glycol/DMSO.

Alternatively, host cells may be transfected or transduced with vectors containing nucleotides encoding an immunoglobulin fusion proteins. Transfected or transduced host cells may be cultured in media. The media may be supplemented with one or more agents for inducing promoters, selecting transfected or transduced cells, or expressing genes encoding the desired sequences.

The expressed immunoglobulin fusion proteins may be secreted into and recovered from the periplasm of the host cells or transported into the culture media. Protein recovery from the periplasm may involve disrupting the host cell. Disruption of the host cell may comprise osmotic shock, sonication or lysis. Centrifugation or filtration may be used to remove cell debris or whole cells. The immunoglobulin fusion proteins may be further purified, for example, by affinity resin chromatography.

Alternatively, immunoglobulin fusion proteins that are secreted into the culture media may be isolated therein. Cells may be removed from the culture and the culture supernatant being filtered and concentrated for further purification of the proteins produced. The expressed polypeptides may be further isolated and identified using commonly known methods such as polyacrylamide gel electrophoresis (PAGE) and Western blot assay.

Immunoglobulin fusion proteins production may be conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (a preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described herein. Cells may be grown for shorter periods prior to induction. Cells are usually induced for about 12-50 hours, although longer or shorter induction time may be used.

To improve the production yield and quality of the immunoglobulin fusion proteins disclosed herein, various fermentation conditions may be modified. For example, to improve the proper assembly and folding of the secreted immunoglobulin fusion proteins polypeptides, additional vectors overexpressing chaperone proteins, such as Dsb proteins (DsbA, DsbB, DsbC, DsbD and or DsbG) or FkpA (a peptidylprolyl cis,trans-isomerase with chaperone activity) may be used to co-transform the host prokaryotic cells. The chaperone proteins have been demonstrated to facilitate the proper folding and solubility of heterologous proteins produced in bacterial host cells.

To minimize proteolysis of expressed heterologous proteins (especially those that are proteolytically sensitive), certain host strains deficient for proteolytic enzymes may be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease VI and combinations thereof. Some E. coli protease-deficient strains are available.

Standard protein purification methods known in the art may be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, hydroxylapatite chromatography, gel electrophoresis, dialysis, and affinity chromatography and gel filtration using, for example, Sephadex G-75.

Immunoglobulin fusion proteins may be concentrated using a commercially available protein concentration filter, for example, an Amicon or Millipore Pellicon® ultrafiltration unit.

Protease inhibitors or protease inhibitor cocktails may be included in any of the foregoing steps to inhibit proteolysis of the immunoglobulin fusion proteins.

In some cases, an immunoglobulin fusion protein may not be biologically active upon isolation. Various methods for “refolding” or converting a polypeptide to its tertiary structure and generating disulfide linkages, may be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, and 2-mercaptoethanol(bME)/di-thio-b(ME). In many instances, a cosolvent may be used to increase the efficiency of the refolding, and common reagents used for this purpose include glycerol, polyethylene glycol of various molecular weights, arginine and the like.

### Compositions

Disclosed herein are compositions comprising an immunoglobulin fusion protein and/or component of an immunoglobulin fusion protein disclosed herein. The compositions may comprise 1, 2, 3, 4, 5, 6, 7, 8, 9, 10 or more immunoglobulin fusion proteins. The immunoglobulin fusion proteins may be different. Alternatively, the immunoglobulin fusion proteins may be the same or similar. The immunoglobulin fusion proteins may comprise different immunoglobulin regions, extender fusion regions, extender peptides, therapeutic peptides or a combination thereof.

The compositions may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; the MONAQUAT™ series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Compositions described herein may be formulated for controlled or sustained delivery in a manner that provides local concentration of the product (e.g., bolus, depot effect) and/or increased stability or half-life in a particular local environment. The compositions may comprise the formulation of immunoglobulin fusion proteins, polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents. See, for example, the description of controlled release porous polymeric microparticles for the delivery of pharmaceutical compositions in WO 93/15722.

Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals.

The immunoglobulin fusion proteins disclosed herein may be microencapsulated.

A pharmaceutical composition disclosed herein can be administered to a subject by any suitable administration route, including but not limited to, parenteral (intravenous, subcutaneous, intraperitoneal, intramuscular, intravascular, intrathecal, intravitreal, infusion, or local), topical, oral, or nasal administration.

Formulations suitable for intramuscular, subcutaneous, peritumoral, or intravenous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent can be optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. The pharmaceutical composition described herein can be in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

Alternatively or additionally, the compositions may be administered locally via implantation into the affected area of a membrane, sponge, or other appropriate material on to which an immunoglobulin fusion protein disclosed herein has been absorbed or encapsulated. Where an implantation device is used, the device may be implanted into any suitable tissue or organ, and delivery of an immunoglobulin fusion protein, nucleic acid, or vector disclosed herein may be directly through the device via bolus, or via continuous administration, or via catheter using continuous infusion.

A pharmaceutical composition comprising an immunoglobulin fusion protein disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations comprising an immunoglobulin fusion protein disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Another preparation may involve an effective quantity of an immunoglobulin fusion protein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size.

Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

The compositions disclosed herein may be useful for providing prognostic or providing diagnostic information.

“Pharmaceutically acceptable” may refer to approved or approvable by a regulatory agency of the Federal or a state government or listed in the U.S. Pharmacopeia or other generally recognized pharmacopeia for use in animals, including humans.

“Pharmaceutically acceptable salt” may refer to a salt of a compound that is pharmaceutically acceptable and that possesses the desired pharmacological activity of the parent compound.

“Pharmaceutically acceptable excipient, carrier or adjuvant” may refer to an excipient, carrier or adjuvant that may be administered to a subject, together with at least one immunoglobulin of the present disclosure, and which does not destroy the pharmacological activity thereof and is nontoxic when administered in doses sufficient to deliver a therapeutic amount of the compound.

“Pharmaceutically acceptable vehicle” may refer to a diluent, adjuvant, excipient, or carrier with which at least one immunoglobulin of the present disclosure is administered.

### Kits

Further disclosed herein are kits which comprise one or more immunoglobulin fusion proteins or components thereof. The immunoglobulin fusion proteins may be packaged in a manner which facilitates their use to practice methods of the present disclosure. For example, a kit comprises an immunoglobulin fusion protein described herein packaged in a container with a label affixed to the container or a package insert that describes use of the immunoglobulin fusion protein in practicing the method. Suitable containers include, for example, bottles, vials, syringes, etc. The containers may be formed from a variety of materials such as glass or plastic. The container may have a sterile access port (for example the container may be an intravenous solution bag or a vial having a stopper pierceable by a hypodermic injection needle). The kit may comprise a container with an immunoglobulin fusion protein contained therein. The kit may comprise a container with (a) an immunoglobulin region of an immunoglobulin fusion protein; (b) an extender fusion region of an immunoglobulin fusion protein; (c) an extender peptide of the extender fusion region; (d) a therapeutic peptide of the extender fusion region; or (e) a combination of a-d. The kit may further comprise a package insert indicating that the first and second compositions may be used to treat a particular condition. Alternatively, or additionally, the kit may further comprise a second (or third) container comprising a pharmaceutically-acceptable buffer (e.g., bacteriostatic water for injection (BWFI), phosphate-buffered saline, Ringer's solution and dextrose solution). It may further comprise other materials desirable from a commercial and user standpoint, including, but not limited to, other buffers, diluents, filters, needles, and syringes. The immunoglobulin fusion protein may be packaged in a unit dosage form. The kit may further comprise a device suitable for administering the immunoglobulin fusion protein according to a specific route of administration or for practicing a screening assay. The kit may contain a label that describes use of the immunoglobulin fusion protein composition.

The composition comprising the immunoglobulin fusion protein may be formulated in accordance with routine procedures as a pharmaceutical composition adapted for intravenous administration to mammals, such as humans, bovines, felines, canines, and murines. Typically, compositions for intravenous administration comprise solutions in sterile isotonic aqueous buffer. Where necessary, the composition may also include a solubilizing agent and/or a local anaesthetics such as lignocaine to ease pain at the site of the injection. Generally, the ingredients may be supplied either separately or mixed together in unit dosage form. For example, the immunoglobulin fusion protein may be supplied as a dry lyophilized powder or water free concentrate in a hermetically sealed container such as an ampoule or sachette indicating the quantity of the immunoglobulin fusion protein. Where the composition is to be administered by infusion, it may be dispensed with an infusion bottle containing sterile pharmaceutical grade water or saline. Where the composition is administered by injection, an ampoule of sterile water for injection or saline may be provided so that the ingredients may be mixed prior to administration.

The amount of the composition described herein which will be effective in the treatment, inhibition and/or prevention of a disease or disorder associated with aberrant expression and/or activity of a therapeutic peptide may be determined by standard clinical techniques. In addition, in vitro assays may optionally be employed to help identify optimal dosage ranges. The precise dose to be employed in the formulation may also depend on the route of administration, and the seriousness of the disease or disorder, and should be decided according to the judgment of the practitioner and each patient's circumstances. Effective doses may be extrapolated from dose-response curves derived from in vitro, animal model test systems or clinical trials.

### Therapeutic Use

Further disclosed herein are immunoglobulin fusion proteins for and methods of treating, alleviating, inhibiting and/or preventing one or more diseases and/or conditions. The method may comprise administering to a subject in need thereof a composition comprising one or more immunoglobulin fusion proteins disclosed herein. The immunoglobulin fusion protein may comprise an immunoglobulin region attached to a therapeutic peptide. In some embodiments, the therapeutic peptide is attached the amino terminus of the immunoglobulin region. The immunoglobulin fusion protein may comprise an immunoglobulin region attached to a non-immunoglobulin region. In some instances, the immunoglobulin fusion protein comprises an immunoglobulin region attached to an extender fusion region, wherein the extender fusion region comprises (a) an extender peptide comprising at least one secondary structure; and (b) a therapeutic peptide. The extender fusion region may be inserted within the antibody region. The extender fusion region may be inserted within an immunoglobulin heavy chain of the antibody region. The extender fusion region may be inserted within an immunoglobulin light chain of the antibody region. The extender fusion region may be conjugated to the antibody region. The extender fusion region may be conjugated to a position within the antibody region. The composition may further comprise a pharmaceutically acceptable carrier. The subject may be a mammal. The mammal may be a human. Alternatively, the mammal is a bovine. The therapeutic peptide may be a peptide or derivative or variant thereof. Alternatively, therapeutic peptide is a small molecule. The therapeutic peptide may be GCSF, bovine GCSF, human GCSF, Moka1, Vm24, Mamba1, 550 peptide, human GLP-1, Exendin-4, human EPO, human FGF21, human GMCSF, human interferon-beta, human interferon-alpha, relaxin, protoxin2, oxyntomodulin, leptin, betatrophin, growth differentiation factor 11 (GDF11), parathyroid hormone, angiopoietin-like 3 (ANGPTL3), IL-11, human growth hormone (hGH), BCCX2, elafin, ZP1, ZPCEX, relaxin, insulin, GLP-2, Ssam6, 550, glucagon or derivative or variant thereof. Alternatively, or additionally, therapeutic peptide is interleukin 8 (IL-8), IL-21, ziconotide, somatostatin, chlorotoxin, SDF1 alpha or derivative or variation thereof. The immunoglobulin region may comprise one or more immunoglobulin domains. The immunoglobulin region may be an immunoglobulin A, an immunoglobulin D, an immunoglobulin E, an immunoglobulin G, or an immunoglobulin M. The immunoglobulin region may be an immunoglobulin heavy chain region or fragment thereof. In some instances, the immunoglobulin region is from a mammalian immunoglobulin. Alternatively, the immunoglobulin region is from a chimeric immunoglobulin. The immunoglobulin region may be from an engineered immunoglobulin or recombinant immunoglobulin. The immunoglobulin region may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region, therapeutic peptide and/or extender fusion region may further comprise one or more linkers. The linker may attach therapeutic peptide to the extender peptide. The linker may attach the extender fusion region to the immunoglobulin region. The linker may attach a proteolytic cleavage site to the immunoglobulin region, extender fusion region, extender peptide, or therapeutic peptide. The linker may be a connecting linker. The connecting linker may connect the therapeutic peptide to the amino terminus of the immunoglobulin region.

The disease or condition may be an autoimmune disease, heteroimmune disease or condition, inflammatory disease, pathogenic infection, thromboembolic disorder, respiratory disease or condition, metabolic disease, central nervous system (CNS) disorder, bone disease or cancer. In other instances, the disease or condition is a blood disorder. In some instances, the disease or condition is obesity, diabetes, osteoporosis, anemia, or pain. In some instances, the disease is heart related, for example, heart failure, acute coronary syndrome, atrial fibrillation, cardiac fibrosis, or coronary artery disease. In some embodiments, the heart failure is non-ischemic acute heart failure, chronic heart failure, acute decompensated heart failure, stable compensated heart failure, acute heart failure, or chronic heart failure. Additional non-limiting examples of disease and conditions include, ischemia reperfusion associated with solid organ transplant (e.g., lung, kidney, liver, heart), cardiopulmonary bypass for organ protection (e.g., renal), ischemic stroke, corneal healing (ocular administration), diabetic nephropathy, cirrhosis, portal hypertension, diabetic would healing, systemic sclerosis, cervical ripening at time of labor, preeclampsia, portal hypertension, and fibrosis.

In some embodiments, the therapeutic peptide is exendin-4 and the disease or condition is obesity, obesity related conditions, diabetes, and/or diabetes related conditions. In some embodiments, the therapeutic peptide is leptin and the disease or condition is obesity, obesity related conditions, diabetes, and/or diabetes related conditions. In some embodiments, the therapeutic peptide is glucagon and the disease or condition is obesity, obesity related conditions, diabetes, and/or diabetes related conditions. In some embodiments, the therapeutic peptide is a glucagon analog, for example ZP1, and the disease or condition is obesity, obesity related conditions, diabetes, and/or diabetes related conditions. In some embodiments, the therapeutic peptide is insulin, and the disease or condition is obesity, obesity related conditions, diabetes, and/or diabetes related conditions. In some embodiments, the therapeutic peptide is oxyntomodulin, and the disease or condition is obesity, obesity related conditions, diabetes, and/or diabetes related conditions. In some embodiments, the therapeutic peptide is a glucagon like protein, for example GLP-1 or GLP-2, and the disease or condition is obesity, obesity related conditions, diabetes, and/or diabetes related conditions.

In some embodiments, the therapeutic peptide is relaxin and the disease or condition is heart failure, heart failure related conditions, fibrosis, and/or fibrosis related conditions. Relaxin includes relaxin2 and relaxins comprising internal linkers such as relaxin2 (XT100), relaxin2 (XT35), relaxin2 (single), relaxin2 (insulin C peptide), relaxin2 (XT21), relaxin2 (30GS), relaxin2 (9GS), and relaxin2 (GGGPRR). In some embodiments, the therapeutic peptide is relaxin and the disease or condition is heart failure, acute coronary syndrome, atrial fibrillation, cardiac fibrosis, or coronary artery disease. In some embodiments, the therapeutic peptide is relaxin and the disease or condition is ischemia reperfusion associated with solid organ transplant (e.g., lung, kidney, liver, heart), cardiopulmonary bypass for organ protection (e.g., renal), ischemic stroke, corneal healing (ocular administration), diabetic nephropathy, cirrhosis, portal hypertension, diabetic would healing, systemic sclerosis, cervical ripening at time of labor, preeclampsia, portal hypertension, or fibrosis.

In some embodiments, the therapeutic peptide is Moka and the disease or condition is an autoimmune disease or autoimmune disease related conditions. The therapeutic peptide may be hGCSF and the disease or condition may be neutropenia. The therapeutic peptide may be hGH and the disease or condition may be a growth disorder. The therapeutic peptide may be IFN-alpha and the disease or condition may be a viral infection. The therapeutic peptide may be the 550 peptide and the disease or condition may be pain. The therapeutic peptide may be Mamba1 and the disease or condition may be pain. The therapeutic peptide may be Ssam6 and the disease or condition may be pain. The therapeutic peptide may be BCCX2 and the disease or condition may be cancer. The therapeutic peptide may be elafin and the disease or condition may be inflammation.

The disease and/or condition may be a chronic disease or condition. Alternatively, the disease and/or condition is an acute disease or condition. The disease or condition may be recurrent, refractory, accelerated, or in remission. The disease or condition may affect one or more cell types. The one or more diseases and/or conditions may be an autoimmune disease, inflammatory disease, cardiovascular disease, metabolic disorder, pregnancy, and cell proliferative disorder.

The disease or condition may be an autoimmune disease. In some cases, the autoimmune disease may be scleroderma, diffuse scleroderma or systemic scleroderma.

The disease or condition may be an inflammatory disease. In some cases, the inflammatory disease may be hepatitis, fibromyalgia or psoriasis.

The disease or condition may be a rheumatic disease. In some cases, the rheumatic disease may be Ankylosing spondylitis, back pain, bursitis, tendinitis, shoulder pain, wrist pain, bicep pain, leg pain, knee pain, ankle pain, hip pain, Achilles pain, Capsulitis, neck pain, osteoarthritis, systemic lupus, erythematosus, rheumatoid arthritis, juvenile arthritis, Sjögren syndrome, Polymyositis, Behçet's disease, Reiter's syndrome, or Psoriatic arthritis. The rheumatic disease may be chronic. Alternatively, the rheumatic disease is acute.

The disease or condition may be a cardiovascular disease. In some cases, the cardiovascular disease may be acute heart failure, congestive heart failure, compensated heart failure, decompensated heart failure, hypercholesterolemia, atherosclerosis, coronary heart disease or ischemic stroke. The cardiovascular disease may be cardiac hypertrophy.

The disease or condition may be a metabolic disorder. In some cases, the metabolic disorder may be hypercholesterolemia, hypobetalipoproteinemia, hypertriglyceridemia, hyperlipidemia, dyslipidemia, ketosis, hypolipidemia, refractory anemia, appetite control, gastric emptying, non-alcoholic fatty liver disease, obesity, type I diabetes mellitus, type II diabetes mellitus, gestational diabetes mellitus, metabolic syndrome. The metabolic disorder may be type I diabetes. The metabolic disorder may be type II diabetes.

The disease or condition may be pregnancy. The immunoglobulin fusion proteins may be used to treat preeclampsia or induce labor.

The disease or condition may be a cell proliferative disorder. The cell proliferative disorder may be a leukemia, lymphoma, carcinoma, sarcoma, or a combination thereof. The cell proliferative disorder may be a myelogenous leukemia, lymphoblastic leukemia, myeloid leukemia, myelomonocytic leukemia, neutrophilic leukemia, myelodysplastic syndrome, B-cell lymphoma, burkitt lymphoma, large cell lymphoma, mixed cell lymphoma, follicular lymphoma, mantle cell lymphoma, Hodgkin lymphoma, recurrent small lymphocytic lymphoma, hairy cell leukemia, multiple myeloma, basophilic leukemia, eosinophilic leukemia, megakaryoblastic leukemia, monoblastic leukemia, monocytic leukemia, erythroleukemia, erythroid leukemia, hepatocellular carcinoma, solid tumors, lymphoma, leukemias, liposarcoma (advanced/metastatic), myeloid malignancy, breast cancer, lung cancer, ovarian cancer, uterine cancer, kidney cancer, pancreatic cancer, and malignant glioma of brain.

Disclosed herein are methods of treating a disease or condition in a subject in need thereof, the method comprising administering to the subject a composition comprising an immunoglobulin fusion protein disclosed herein. In some embodiments, the immunoglobulin fusion protein comprises a therapeutic peptide attached to an immunoglobulin region. In some embodiments, the therapeutic peptide is attached to the immunoglobulin region via a chemical linker referred to as a connecting peptide. In some embodiments, the therapeutic peptide is attached to the amino terminus of the immunoglobulin region. In some embodiments, the therapeutic peptide is oxyntomodulin. In some embodiments, the therapeutic peptide is insulin. In some embodiments, the therapeutic peptide is exendin-4. In some embodiments, the therapeutic peptide is a glucagon analog. The disease or condition may be a metabolic disorder. The metabolic disorder may be diabetes. Diabetes may be type II diabetes mellitus. Diabetes may be type I diabetes. The metabolic disorder may be obesity. Additional metabolic disorders include, but are not limited to, metabolic syndrome, appetite control or gastric emptying.

Disclosed herein are methods of treating a disease or condition in a subject in need thereof, the method comprising administering to the subject a composition comprising an immunoglobulin fusion protein disclosed herein. In some embodiments, the immunoglobulin fusion protein comprises a therapeutic peptide attached to an immunoglobulin region. In some embodiments, the therapeutic peptide is attached to the immunoglobulin region via a chemical linker referred to as a connecting peptide. In some embodiments, the therapeutic peptide is attached to the amino terminus of the immunoglobulin region. In some embodiments, the therapeutic peptide is relaxin. The disease or condition may be a cardiovascular disease. The cardiovascular disease may be acute heart failure. Additional cardiovascular diseases include, but are not limited to, congestive heart failure, compensated heart failure or decompensated heart failure. The disease or condition may be an autoimmune disorder. The autoimmune disorder may be scleroderma, diffuse scleroderma or systemic scleroderma. The disease or condition may be an inflammatory disease. The inflammatory disease may be fibromyalgia. The disease or condition may be fibrosis. Alternatively, the disease or condition is pregnancy. The immunoglobulin fusion protein may be used to treat preeclampsia or induce labor.

Further disclosed herein are methods of treating a disease or condition in a subject in need thereof, the method comprising administering to the subject a comprising an immunoglobulin fusion protein disclosed herein. The immunoglobulin fusion protein may comprise an immunoglobulin region attached to a non-immunoglobulin region. The non-immunoglobulin region may comprise leptin. In some instances, the immunoglobulin fusion protein comprises an immunoglobulin region attached to an extender fusion region, wherein the extender fusion region comprises an extender peptide and a therapeutic peptide, wherein the therapeutic peptide is leptin. The disease or condition may be a metabolic disorder. The metabolic disorder may be obesity. The metabolic disorder may be diabetes. Diabetes may be type 2 diabetes mellitus, type I diabetes mellitus or gestational diabetes mellitus. Additional metabolic disorders include, but are not limited to, appetite control and nonalcoholic fatty liver disease. The disease or condition may be a cell proliferative disorder. The cell proliferative disorder may be breast cancer. The condition may be leptin deficiency in individuals with congenital generalized or acquired generalized lipodystrophy.

Disclosed herein may be a method of preventing or treating a disease or condition in a subject in need thereof comprising administering to the subject a composition comprising one or more immunoglobulin fusion proteins disclosed herein. The immunoglobulin fusion protein may comprise an immunoglobulin region attached to therapeutic peptide. The immunoglobulin fusion protein may comprise one or more immunoglobulin heavy chains, light chains, or a combination thereof. The immunoglobulin fusion protein sequence may share 50%, 60%, 70%, 80%, 85%, 90%, 95%, 97%, 99%, or more amino acid sequence identity to a heavy chain sequence provided by SEQ ID NOs: 43, 44, 50, 192, 195-198, 201-213, 216-220, 222, 266. The immunoglobulin fusion protein sequence may share 50%, 60%, 70%, 80%, 85%, 90%, 95%, 97%, 99%, or more amino acid sequence identity to a light chain sequence provided by SEQ ID NOs: 42, 45-49, 51-74, 193, 194, 199, 200, 214, 215, 221. The immunoglobulin heavy chain may be encoded by a nucleotide sequence that is at least about 50%, 60%, 70%, 80%, 85%, 90%, 95%, 97%, 99%, or more homologous to SEQ ID NOs: 10-11, 17, 161, 164-167, 170-182, 185-189, 191, 265. The immunoglobulin light chain may be encoded by a nucleotide sequence that is at least about 50%, 60%, 70%, 80%, 85%, 90%, 95%, 97%, 99%, or more homologous to SEQ ID NOs: 9, 12-16, 18-41, 162, 163, 168, 169, 183, 184, 190. The immunoglobulin fusion protein may further comprise one or more linkers. The immunoglobulin fusion protein may further comprise one or more internal linkers. The immunoglobulin fusion protein may further comprise one or more proteolytic cleavage sites. The disease or condition may be an autoimmune disease, heteroimmune disease or condition, inflammatory disease, pathogenic infection, thromboembolic disorder, respiratory disease or condition, metabolic disease, central nervous system (CNS) disorder, bone disease or cancer. The disease or condition may be a blood disorder. In some instances, the disease or condition may be obesity, diabetes, osteoporosis, anemia, or pain. In some embodiments, the disease or condition is heart failure, acute coronary syndrome, atrial fibrillation, cardiac fibrosis, or coronary artery disease. In some embodiments, the disease or condition is ischemia reperfusion associated with solid organ transplant (e.g., lung, kidney, liver, heart), cardiopulmonary bypass for organ protection (e.g., renal), ischemic stroke, corneal healing (ocular administration), diabetic nephropathy, cirrhosis, portal hypertension, diabetic would healing, systemic sclerosis, cervical ripening at time of labor, preeclampsia, portal hypertension, or fibrosis.

Disclosed herein is a method of preventing or treating an autoimmune disease in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be Moka1 or a derivative or variant thereof. The therapeutic peptide may be VM-24 or a derivative or variant thereof. The therapeutic peptide may be beta-interferon or a derivative or variant thereof. The immunoglobulin fusion protein or immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. The mammalian immunoglobulin may be a murine immunoglobulin. The immunoglobulin fusion protein, immunoglobulin region or therapeutic peptide may further comprise a linker. The linker may attach Moka1, VM-24, beta-interferon, or a derivative or variant thereof to the immunoglobulin region. The autoimmune disease may be a T-cell mediated autoimmune disease. T-cell mediated autoimmune diseases include, but are not limited to, multiple sclerosis, type-1 diabetes, and psoriasis. In other instances, the autoimmune disease lupus, Sjogren's syndrome, scleroderma, rheumatoid arthritis, dermatomyositis, Hasmimoto's thyroiditis, Addison's disease, celiac disease, Crohn's disease, pernicious anemia, pemphigus vulgaris, vitiligo, autoimmune hemolytic anemia, idiopathic thrombocytopenic purpura, myasthenia gravis, Ord's thyroiditis, Graves' disease, Guillain-Barre syndrome, acute disseminated encephalomyelitis, opsoclonus-myoclonus syndrome, ankylosing spondylitisis, antiphospholipid immunoglobulin syndrome, aplastic anemia, autoimmune hepatitis, Goodpasture's syndrome, Reiter's syndrome, Takayasu's arteritis, temporal arteritis, Wegener's granulomatosis, alopecia universalis, Behcet's disease, chronic fatigue, dysautonomia, endometriosis, interstitial cystitis, neuromyotonia, scleroderma, and vulvodynia. Lupus can include, but may be not limited to, acute cutaneous lupus erythematosus, subacute cutaneous lupus erythematosus, chronic cutaneous lupus erythematosus, discoid lupus erythematosus, childhood discoid lupus erythematosus, generalized discoid lupus erythematosus, localized discoid lupus erythematosus, chilblain lupus erythematosus (hutchinson), lupus erythematosus-lichen planus overlap syndrome, lupus erythematosus panniculitis (lupus erythematosus profundus), tumid lupus erythematosus, verrucous lupus erythematosus (hypertrophic lupus erythematosus), complement deficiency syndromes, drug-induced lupus erythematosus, neonatal lupus erythematosus, and systemic lupus erythematosus. The disease or condition may be multiple sclerosis. The disease or condition may be diabetes.

Further disclosed herein is a method of preventing or treating a disease or condition which would benefit from the modulation of a potassium voltage-gated channel in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The potassium voltage-gated channel may be a KCNA3 or Kᵥ1.3 channel. The subject may be a mammal. The mammal may be a human. Alternatively, the mammal may be a bovine. The therapeutic peptide may be Moka1 or a derivative or variant thereof. The therapeutic peptide may be VM24 or a derivative or variant thereof. The immunoglobulin fusion protein or immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach Moka1, VM-24, or a derivative or variant thereof to the immunoglobulin region. The disease or condition may be an autoimmune disease. The autoimmune disease may be a T-cell mediated autoimmune disease. The disease or condition may be episodic ataxia, seizure, or neuromyotonia. Modulating a potassium voltage-gated channel may comprise inhibiting or blocking a potassium voltage-gated channel. Modulating a potassium voltage-gated channel may comprise activating a potassium voltage-gated channel.

Provided herein is a method of preventing or treating a metabolic disease or condition in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be GLP-1, Exendin-4, FGF21 or a derivative or variant thereof. The GLP-1 may be a human GLP-1. The FGF21 may be a human FGF21. The immunoglobulin or immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach GLP-1, Exendin-4, FGF21, or a derivative or variant thereof to the immunoglobulin region. Metabolic diseases and/or conditions may include disorders of carbohydrate metabolism, amino acid metabolism, organic acid metabolism (organic acidurias), fatty acid oxidation and mitochondrial metabolism, porphyrin metabolism, purine or pyrimidine metabolism, steroid metabolism, mitochondrial function, peroxisomal function, urea cycle disorder, urea cycle defects or lysosomal storage disorders. The metabolic disease or condition may be diabetes. In other instances, the metabolic disease or condition may be glycogen storage disease, phenylketonuria, maple syrup urine disease, glutaric acidemia type 1, Carbamoyl phosphate synthetase I deficiency, alcaptonuria, Medium-chain acyl-coenzyme A dehydrogenase deficiency (MCADD), acute intermittent porphyria, Lesch-Nyhan syndrome, lipoid congenital adrenal hyperplasia, congenital adrenal hyperplasia, Kearns-Sayre syndrome, Zellweger syndrome, Gaucher's disease, or Niemann Pick disease.

Provided herein is a method of preventing or treating a central nervous system (CNS) disorder in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be GLP-1, Exendin-4 or a derivative or variant thereof. The GLP-1 may be a human GLP-1. The immunoglobulin may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach GLP-1, Exendin-4, or a derivative or variant thereof to the immunoglobulin region. The CNS disorder may be Alzheimer's disease (AD). Additional CNS disorders include, but are not limited to, encephalitis, meningitis, tropical spastic paraparesis, arachnoid cysts, Huntington's disease, locked-in syndrome, Parkinson's disease, Tourette's, and multiple sclerosis.

Provided herein is a method of preventing or treating a disease or condition which benefits from a GLP-1R and/or glucagon receptor (GCGR) agonist in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be GLP-1, Exendin-4 or a derivative or variant thereof. The GLP-1 may be a human GLP-1. The immunoglobulin fusion protein or immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach GLP-1, Exendin-4, or a derivative or variant thereof to the immunoglobulin region. The disease or condition may be a metabolic disease or disorder. The disease or condition may be diabetes. In other instances, the disease or condition may be obesity. Additional diseases and/or conditions which benefit from a GLP-1R and/or GCGR agonist include, but are not limited to, dyslipidemia, cardiovascular and fatty liver diseases.

Provided herein is a method of preventing or treating a blood disorder in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be erythropoietin, GMCSF or a derivative or variant thereof. The erythropoietin may be a human erythropoietin. The GMCSF may be a human GMCSF. The immunoglobulin may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach erythropoietin, GMCSF, or a derivative or variant thereof to the immunoglobulin region. The blood disorder may be anemia. Examples of anemia include, but are not limited to, hereditary xerocytosis, congenital dyserythropoietic anemia, Rh null disease, infectious mononucleosis related anemia, drugs-related anemia, aplastic anemia, microcytic anemia, macrocytic anemia, normocytic anemia, hemolytic anemia, poikilocytic anemia, spherocytic anemia, drepanocytic anemia, normochromic anemia, hyperchromic anemia, hypochromic anemia, macrocytic-normochromic anemia, microcytic-hypochromic anemia, normocytic-normochromic anemia, iron-deficiency anemia, pernicious anemia, folate-deficiency anemia, thalassemia, sideroblastic anemia, posthemorrhagic anemia, sickle cell anemia, chronic anemia, achrestic anemia, autoimmune haemolytic anemia, Cooley's anemia, drug-induced immune haemolytic anemia, erythroblastic anemia, hypoplastic anemia, Diamond-Blackfan anemia, Pearson's anemia, transient anemia, Fanconi's anemia, Lederer's anemia, myelpathic anemia, nutritional anemia, spur-cell anemia, Von Jaksh's anemia, sideroblatic anemia, sideropenic anemia, alpha thalassemia, beta thalassemia, hemoglobin h disease, acute acquired hemolytic anemia, warm autoimmune hemolytic anemia, cold autoimmune hemolytic anemia, primary cold autoimmune hemolytic anemia, secondary cold autoimmune hemolytic anemia, secondary autoimmune hemolytic anemia, primary autoimmune hemolytic anemia, x-linked sideroblastic anemia, pyridoxine-responsive anemia, nutritional sideroblastic anemia, pyridoxine deficiency-induced sideroblastic anemia, copper deficiency-induced sideroblastic anemia, cycloserine-induced sideroblastic anemia, chloramphenicol-induced sideroblastic anemia, ethanol-induced sideroblastic anemia, isoniazid-induced sideroblastic anemia, drug-induced sideroblastic anemia, toxin-induced sideroblastic anemia, microcytic hyperchromic anemia, macrocytic hyperchromic anemia, megalocytic-normochromic anemia, drug-induced immune hemolytic anemia, non-hereditary spherocytic anemia, inherited spherocytic anemia, and congenital spherocytic anemia. In other instances, the blood disorder may be malaria. Alternatively, the blood disorder may be lymphoma, leukemia, multiple myeloma, or myelodysplastic syndrome. The blood disorder may be neutropenia, Shwachmann-Daimond syndrome, Kostmann syndrome, chronic granulomatous disease, leukocyte adhesion deficiency, meyloperoxidase deficiency, or Chediak Higashi syndrome.

Provided herein is a method of preventing or treating a disease or disorder which benefits from stimulating or increasing white blood cell production in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be GMCSF or a derivative or variant thereof. The GMCSF may be a human GMCSF. The immunoglobulin fusion protein or immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach the immunoglobulin region to the immunoglobulin region. The disease or disorder may be neutropenia, Shwachmann-Daimond syndrome, Kostmann syndrome, chronic granulomatous disease, leukocyte adhesion deficiency, meyloperoxidase deficiency, or Chediak Higashi syndrome.

Provided herein is a method of preventing or treating a disease or disorder which benefits from stimulating or increasing red blood cell production in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be erythropoietinor a derivative or variant thereof. The erythropoietin may be a human erythropoietin. The immunoglobulin may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach erythropoietin, or a derivative or variant thereof to the immunoglobulin region. The disease or disorder may be anemia.

Provided herein is a method of preventing or treating obesity in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be GLP-1 or a derivative or variant thereof. The GLP-1 may be a human GLP-1. The therapeutic peptide may be FGF21 or a derivative or variant thereof. The FGF21 may be a human FGF21. The therapeutic peptide may be Exendin-4 or a derivative or variant thereof. The immunoglobulin may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach GLP-1, Exendin-4, FGF21, or a derivative or variant thereof to the immunoglobulin region.

Provided herein is a method of preventing or treating a pain in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The subject may be a mammal. In certain instances, the mammal may be a human. Alternatively, the mammal may be a bovine. The therapeutic peptide may be a protoxin2 or a derivative or variant thereof. The therapeutic peptide may be a 550 peptide or a derivative or variant thereof. The therapeutic peptide may be a Mamba1 or a derivative or variant thereof. The immunoglobulin fusion proteins, immunoglobulin regions, and/or therapeutic peptide may further comprise one or more linkers. The linker may attach the protoxin2, 550 peptide, Mamba1 or a derivative or variant thereof to the immunoglobulin region.

Provided herein is a method of preventing or treating a disease or condition which benefits from modulating a sodium ion channel in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The subject may be a mammal. In certain instances, the mammal may be a human. Alternatively, the mammal may be a bovine. The therapeutic peptide may be protoxin2 or a derivative or variant thereof. The therapeutic peptide may be a 550 peptide or a derivative or variant thereof. The one or more antibodies, immunoglobulin fragments, or immunoglobulin constructs further comprise a linker. The linker may attach the therapeutic peptide to the immunoglobulin region. The sodium ion channel may be a Naᵥ channel. The Naᵥ channel may be a Naᵥ1.7 channel. Modulating a sodium ion channel may comprise inhibiting or blocking a sodium ion channel. Modulating a sodium ion channel may comprise activating a sodium ion channel. The disease or condition may be Dravet Syndrome, generalized epilepsy with febrile seizures plus (GEFS+), paramyotonia congenital or erythromelalgia. The disease or condition may be pain.

Provided herein is a method of preventing or treating a disease or condition which benefits from modulating an acid sensing ion channel (ASIC) in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The subject may be a mammal. In certain instances, the mammal may be a human. Alternatively, the mammal may be a bovine. The therapeutic peptide may be protoxin2 or a derivative or variant thereof. The therapeutic peptide may be Mamba 1 or a derivative or variant thereof. The one or more antibodies, immunoglobulin fragments, or immunoglobulin constructs further comprise a linker. The linker may attach the therapeutic peptide to the immunoglobulin region. Modulating an ASIC may comprise inhibiting or blocking the ASIC. Modulating an ASIC may comprise activating the ASIC. The disease or condition may be a central nervous system disorder. In other instances, the disease or condition is pain.

Provided herein is a method of preventing or treating a pathogenic infection in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be alpha-interferon or a derivative or variant thereof. The therapeutic peptide may be beta-interferon or a derivative or variant thereof. The immunoglobulin may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach alpha-interferon, beta-interferon, or a derivative or variant thereof to the immunoglobulin region. The pathogenic infection may be a bacterial infection. The pathogenic infection may be a fungal infection. The pathogenic infection may be a parasitic infection. The pathogenic infection may be a viral infection. The viral infection may be a herpes virus.

Provided herein is a method of preventing or treating a cancer in a subject in need thereof comprising administering to the subject a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The composition may further comprise a pharmaceutically acceptable carrier. The subject may be a mammal. The mammal may be a human. Alternatively, the mammal may be a bovine. The therapeutic peptide may be beta-interferon or a derivative or variant thereof. The therapeutic peptide may be BCCX2 or a derivative or variant thereof. The immunoglobulin may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. The immunoglobulin domain may be from a mammalian immunoglobulin. Alternatively, the immunoglobulin domain may be from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin may be a murine immunoglobulin. The immunoglobulin fusion protein, immunoglobulin region, and/or therapeutic peptide may further comprise one or more linkers. The linker may attach beta-interferon, BCCX2 or a derivative or variant thereof to the immunoglobulin region. The cancer may be a hematological malignancy. The hematological malignancy may be a leukemia or lymphoma. The hematological malignancy may be a B-cell lymphoma, T-cell lymphoma, follicular lymphoma, marginal zone lymphoma, hairy cell leukemia, chronic myeloid leukemia, mantle cell lymphoma, nodular lymphoma, Burkitt's lymphoma, cutaneous T-cell lymphoma, chronic lymphocytic leukemia, or small lymphocytic leukemia.

Provided herein is a method of preventing or treating a disease or condition which would benefit from modulation of a receptor in a subject in need thereof comprising administering to the subject a composition disclosed herein. The immunoglobulin fusion protein may comprise an immunoglobulin region attached to a therapeutic peptide. In some embodiments, the therapeutic peptide is attached to the amino terminus of an immunoglobulin region. In some instances, the immunoglobulin fusion protein comprises one or more immunoglobulin fusion proteins comprising an immunoglobulin region attached to a therapeutic peptide. The subject may be a mammal. In certain instances, the mammal may be a human. Alternatively, the mammal may be a bovine. The therapeutic peptide may be hGCSF or a derivative or variant thereof and the receptor may be GCSFR. The therapeutic peptide may be erythropoeitin or a derivative or variant thereof and the receptor may be EPOR. The therapeutic peptide may be Exendin-4 or a derivative or variant thereof and the receptor may be GLP1R. The therapeutic peptide may be GLP-1 or a derivative or variant thereof and the receptor may be GLP1R. The therapeutic peptide may be leptin or a derivative or variant thereof and the receptor may be LepR. The therapeutic peptide may be hGH or a derivative or variant thereof and the receptor may be GHR. The therapeutic peptide may be interferon-alpha or a derivative or variant thereof and the receptor may be IFNR. The therapeutic peptide may be interferon-beta or a derivative or variant thereof and the receptor may be IFNR. The therapeutic peptide may be relaxin or a derivative or variant thereof and the receptor may be LGR7. The therapeutic peptide may be BCCX2 or a derivative or variant thereof and the receptor may be CXCR4. The therapeutic peptide may be GMCSF or a derivative or variant thereof and the receptor may be GMCSFR. The one or more immunoglobulin fusion proteins, therapeutic peptides, or immunoglobulin regions further comprise a linker. The linker may attach the therapeutic peptide to the immunoglobulin region. The disease or condition may be an autoimmune disease. The autoimmune disease may be a T-cell mediated autoimmune disease. The disease or condition may be a metabolic disorder. The metabolic disorder may be diabetes. The disease or condition may be an inflammatory disorder. The inflammatory disorder may be multiple sclerosis. The disease or condition may be a cell proliferative disorder. The disease or condition may be a blood disorder. The blood disorder may be neutropenia. The blood disorder may be anemia. The disease or condition may be a pathogenic infection. The pathogenic infection may be a viral infection. The disease or condition may be a growth disorder. The disease or condition may be a cardiovascular condition. The cardiovascular condition may be acute heart failure. Modulating the receptor may comprise inhibiting or blocking the receptor. Modulating the receptor may comprise activating the receptor. The therapeutic peptide may act as a receptor agonist. The therapeutic peptide may act as a receptor antagonist.

Provided herein is a method of preventing or treating a disease in a mammal in need thereof comprising administering a pharmaceutical composition described herein to said mammal. In some embodiments, the disease may be an infectious disease. In certain embodiments, the infectious disease may be mastitis. In some embodiments, the infectious disease may be a respiratory disease. In certain embodiments, the respiratory disease may be bovine respiratory disease of shipping fever. In certain embodiments, the mammal in need may be a dairy animal selected from a list comprising cow, camel, donkey, goat, horse, reindeer, sheep, water buffalo, moose and yak. In some embodiments, the mammal in need may be bovine.

Provided herein is a method of preventing or treating mastitis in a dairy animal, comprising providing to said dairy animal an effective amount of a composition comprising one or more immunoglobulin fusion proteins disclosed herein. The immunoglobulin fusion protein may comprise an immunoglobulin region attached to a therapeutic peptide. In some embodiments, the therapeutic peptide is attached to the amino terminus of an immunoglobulin region. The therapeutic peptide may be GCSF. The GCSF may be a bovine GCSF. The GCSF may be a human GCSF. In some embodiments, the dairy animal may be a cow or a water buffalo.

Provided are methods of treatment, inhibition and prevention of a disease or condition in a subject in need thereof by administration to the subject of an effective amount of an immunoglobulin fusion protein or pharmaceutical composition described herein. The immunoglobulin fusion protein may be substantially purified (e.g., substantially free from substances that limit its effect or produce undesired side-effects). The subject may be an animal, including but not limited to animals such as cows, pigs, sheep, goats, rabbits, horses, chickens, cats, dogs, mice, etc. The subject may be a mammal. The subject may be a human. The subject may be a non-human primate. Alternatively, the subject may be a bovine. The subject may be an avian, reptile or amphibian.

### Additional Uses

Further disclosed herein are uses of an immunoglobulin fusion protein in the manufacture of a medicament for the treatment of a disease or condition. The immunoglobulin fusion protein may be any of the immunoglobulin fusion proteins disclosed herein. Disclosed herein is the use of an immunoglobulin fusion protein in the manufacture of a medicament for the treatment of a disease or condition, the immunoglobulin fusion protein comprising an immunoglobulin region attached to a therapeutic peptide. In some embodiments, the therapeutic peptide is attached to the amino terminus of an immunoglobulin region. Further disclosed herein is the use of an immunoglobulin fusion protein in the manufacture of a medicament for the treatment of a disease or condition, the immunoglobulin fusion protein comprising an immunoglobulin region attached to a therapeutic peptide. In some embodiments, the therapeutic peptide is attached to the amino terminus of an immunoglobulin region. The immunoglobulin fusion protein may comprise one or more internal linkers, one or more protease cleavage sites, one or more connecting peptides, one or more extender peptides, and any combination thereof. The one or more internal linkers, one or more protease cleavage sites, one or more connecting peptides, and/or one or more extender peptides may be inserted within the immunoglobulin region. The one or more internal linkers, one or more protease cleavage sites, one or more connecting peptides, and/or one or more extender peptides may be inserted within the therapeutic peptide. The one or more internal linkers, one or more protease cleavage sites, one or more connecting peptides, and/or one or more extender peptides may be connected to the amino terminus of the immunoglobulin region. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The therapeutic peptide may be a peptide or derivative or variant thereof. Alternatively, therapeutic peptide is a small molecule. The therapeutic peptide may comprise GCSF. The GCSF may comprise a human GCSF. The therapeutic peptide may comprise Moka1. The therapeutic peptide may comprise VM24. The therapeutic peptide may comprise Exendin-4. The therapeutic peptide may comprise erythropoietin. The erythropoietin may comprise a human erythropoeitin. The therapeutic peptide may comprise leptin. The therapeutic peptide may comprise insulin. The therapeutic peptide may comprise Ssam6. The therapeutic peptide may comprise oxyntomodulin. The therapeutic peptide may comprise a growth hormone (GH). The growth hormone may be a human growth hormone (hGH). The therapeutic peptide may comprise interferon-alpha. The therapeutic peptide may comprise a glucagon analog. The therapeutic peptide may comprise interferon-beta. The therapeutic peptide may comprise GLP-1. The therapeutic peptide may comprise GLP-2. The therapeutic peptide may comprise relaxin. The therapeutic peptide may comprise a 550 peptide. The therapeutic peptide may comprise Mamba1. The therapeutic peptide may comprise BCCX2. The therapeutic peptide may comprise elafin. The therapeutic peptide may comprise betatrophin. The therapeutic peptide may comprise GDF11. The therapeutic peptide may comprise GMCSF. The therapeutic peptide may comprise glucagon. The disease or condition may be an autoimmune disease, heteroimmune disease or condition, inflammatory disease, pathogenic infection, thromboembolic disorder, respiratory disease or condition, metabolic disease, central nervous system (CNS) disorder, bone disease or cancer. In other instances, the disease or condition is a blood disorder. In some instances, the disease or condition is obesity, diabetes, osteoporosis, anemia, or pain. The disease or condition may be a growth disorder. In some embodiments, the disease or condition is heart failure, acute coronary syndrome, atrial fibrillation, cardiac fibrosis, or coronary artery disease. In some embodiments, the disease or condition is ischemia reperfusion associated with solid organ transplant (e.g., lung, kidney, liver, heart), cardiopulmonary bypass for organ protection (e.g., renal), ischemic stroke, corneal healing (ocular administration), diabetic nephropathy, cirrhosis, portal hypertension, diabetic would healing, systemic sclerosis, cervical ripening at time of labor, preeclampsia, portal hypertension, or fibrosis.

Disclosed herein is the use of an immunoglobulin fusion protein in the manufacture of a medicament for the treatment of a cell proliferative disorder. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The cell proliferative disorder may be cancer. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptides may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be BCCX2.

Disclosed herein is the use of an immunoglobulin fusion protein in the manufacture of a medicament for the treatment of a metabolic disorder. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The metabolic disorder may be diabetes. Diabetes may be type I diabetes. Diabetes may be type II diabetes. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may be Exendin-4. The therapeutic peptide may be GLP-1. The therapeutic peptide may be leptin. The therapeutic peptide may be betatrophin.

Disclosed herein is the use of an immunoglobulin fusion protein in the manufacture of a medicament for the treatment of an autoimmune disease or condition. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may be Moka1. The therapeutic peptide may be VM24.

Disclosed herein is the use of an immunoglobulin fusion protein in the manufacture of a medicament for the treatment of an inflammatory disease or condition. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The inflammatory disease or condition may be multiple sclerosis. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be elafin. The therapeutic peptide may be interferon-beta.

Disclosed herein is the use of an immunoglobulin fusion protein in the manufacture of a medicament for the treatment of a disease or condition of the central nervous system. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The disease or condition of the central nervous system may be pain. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic region may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may be a 550 peptide. The therapeutic peptide may be Mamba1.

Disclosed herein is the use of an immunoglobulin fusion protein in the manufacture of a medicament for the treatment of a cardiovascular disease or condition. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The cardiovascular disease or condition may be acute heart failure. The cardiovascular disease or condition may be cardiac hypertrophy. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be relaxin. The therapeutic peptide may be GDF11.

Disclosed herein is the use of an immunoglobulin fusion protein in the manufacture of a medicament for the treatment of a hematological disease or condition. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The hematological disease or condition may be anemia. The hematological disease or condition may be neutropenia. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may be GCSF. The GCSF may be a human GCSF. The therapeutic peptide may be erythropoietin. The erythropoietin may be a human erythropoietin. The therapeutic peptide may be GMCSF.

Disclosed herein is the use of an immunoglobulin fusion protein in the manufacture of a medicament for the treatment of a pathogenic infection. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The pathogenic infection may be a viral infection. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be interferon-alpha.

Disclosed herein is the use of an immunoglobulin fusion protein in the manufacture of a medicament for the treatment of a growth disorder. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. Examples of growth disorders included, but are not limited to, achondroplasia, achondroplasia in children, acromegaly, adiposogenital dystrophy, dwarfism, gigantism, Brooke Greenberg, hemihypertrophy, hypochondroplasia, Jansen's metaphyseal chondrodysplasia, Kowarski syndrome, Léri-Weill dyschondrosteosis, local gigantism, macrodystrophia lipomatosa, Majewski's polydactyly syndrome, microcephalic osteodysplastic primordial dwarfism type II, midget, overgrowth syndrome, parastremmatic dwarfism, primordial dwarfism, pseudoachondroplasia, psychosocial short stature, Seckel syndrome, short rib-polydactyly syndrome and Silver-Russell syndrome. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may be a growth hormone. The growth hormone may be a human growth hormone (hGH).

Further disclosed herein are uses of an immunoglobulin fusion protein for the treatment of a disease or condition. Disclosed herein is the use of an immunoglobulin fusion protein for the treatment of a disease or condition in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may comprise GCSF. The GCSF may be a human GCSF. The therapeutic peptide may be Moka1. The therapeutic peptide may be VM24. The therapeutic peptide may be Exendin-4. The therapeutic peptide may be erythropoietin. The erythropoietin may be a human erythropoeitin. The therapeutic peptide may be leptin. The therapeutic peptide may be a growth hormone (GH). The growth hormone may be a human growth hormone (hGH). The therapeutic peptide may be interferon-alpha. The therapeutic peptide may be interferon-beta. The therapeutic peptide may be GLP-1. The therapeutic peptide may be relaxin. The therapeutic peptide may be a 550 peptide. The therapeutic peptide may be Mamba1. The therapeutic peptide may be BCCX2. The therapeutic peptide may be elafin. The therapeutic peptide may be betatrophin. The therapeutic peptide may be GDF11. The therapeutic peptide may be GMCSF. The disease or condition may be an autoimmune disease, heteroimmune disease or condition, inflammatory disease, pathogenic infection, thromboembolic disorder, respiratory disease or condition, metabolic disease, central nervous system (CNS) disorder, bone disease or cancer. In other instances, the disease or condition is a blood disorder. In some instances, the disease or condition is obesity, diabetes, osteoporosis, anemia, or pain. The disease or condition may be a growth disorder.

Disclosed herein is the use of an immunoglobulin fusion protein for the treatment of a cell proliferative disorder in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach therapeutic peptide to the immunoglobulin region. The therapeutic peptide may be a peptide or derivative or variant thereof. Alternatively, therapeutic peptide is a small molecule. The therapeutic peptide may be BCCX2.

Disclosed herein is the use of an immunoglobulin fusion protein for the treatment of a metabolic disorder in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The metabolic disorder may be diabetes. Diabetes may be type I diabetes. Diabetes may be type II diabetes. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be Exendin-4. The therapeutic peptide may be GLP-1. The therapeutic peptide may be leptin. The therapeutic peptide may be betatrophin.

Disclosed herein is the use of an immunoglobulin fusion protein for the treatment of an autoimmune disease or condition in a subject in need thereof. The immunoglobulin fusion protein may be any of the immunoglobulin fusion proteins disclosed herein. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be Moka1. The therapeutic peptide may be VM24.

Disclosed herein is the use of an immunoglobulin fusion protein for the treatment of an inflammatory disease or condition in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The inflammatory disease or condition may be multiple sclerosis. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be elafin. The therapeutic peptide may be interferon-beta.

Disclosed herein is the use of an immunoglobulin fusion protein for the treatment of a disease or condition of the central nervous system in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The disease or condition of the central nervous system may be pain. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be a 550 peptide. The therapeutic peptide may be Mamba1.

Disclosed herein is the use of an immunoglobulin fusion protein for the treatment of a cardiovascular disease or condition in a subject in need thereof. In some embodiments, the immunoglobulin fusion protein treats a disease or condition selected from heart failure, acute coronary syndrome, atrial fibrillation, cardiac fibrosis, and coronary artery disease. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The cardiovascular disease or condition may be acute heart failure. The cardiovascular disease or condition may be cardiac hypertrophy. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be relaxin. The therapeutic peptide may be GDF11.

Disclosed herein is the use of an immunoglobulin fusion protein for the treatment of a hematological disease or condition in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The hematological disease or condition may be anemia. The hematological disease or condition may be neutropenia. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be GCSF. The GCSF may be a human GCSF. The therapeutic peptide may be erythropoietin. The erythropoietin may be a human erythropoietin. The therapeutic peptide may be GMCSF.

Disclosed herein is the use of an immunoglobulin fusion protein for the treatment of a pathogenic infection in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. The pathogenic infection may be a viral infection. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be interferon-alpha.

Disclosed herein is the use of an immunoglobulin fusion protein for the treatment of a growth disorder in a subject in need thereof. The immunoglobulin fusion protein may be any of the immunoglobulin fusion proteins disclosed herein. The immunoglobulin fusion protein may comprise an immunoglobulin region attached to one or more therapeutic peptides. In some embodiments, the therapeutic peptide is attached the amino terminus of the immunoglobulin region. Examples of growth disorders included, but are not limited to, achondroplasia, achondroplasia in children, acromegaly, adiposogenital dystrophy, dwarfism, gigantism, Brooke Greenberg, hemihypertrophy, hypochondroplasia, Jansen's metaphyseal chondrodysplasia, Kowarski syndrome, Léri-Weill dyschondrosteosis, local gigantism, macrodystrophia lipomatosa, Majewski's polydactyly syndrome, microcephalic osteodysplastic primordial dwarfism type II, midget, overgrowth syndrome, parastremmatic dwarfism, primordial dwarfism, pseudoachondroplasia, psychosocial short stature, Seckel syndrome, short rib-polydactyly syndrome and Silver-Russell syndrome. The immunoglobulin region may comprise one or more immunoglobulin domains. The immunoglobulin domain may be an immunoglobulin A, an immunoglobulin D, an immunoglobulin E, an immunoglobulin G, or an immunoglobulin M. The immunoglobulin domain may be an immunoglobulin heavy chain region or fragment thereof. The immunoglobulin domain may be an immunoglobulin light chain region or fragment thereof. The immunoglobulin domain may be from an anti-viral, anti-bacterial, anti-parasitic, and/or anti-fungal immunoglobulin. In some instances, the immunoglobulin domain is from a mammalian immunoglobulin. Alternatively, the immunoglobulin domain is from a chimeric immunoglobulin. The immunoglobulin domain may be from an engineered immunoglobulin or recombinant immunoglobulin. The immunoglobulin domain may be from a humanized, human engineered or fully human immunoglobulin. The mammalian immunoglobulin may be a bovine immunoglobulin. The mammalian immunoglobulin may be a human immunoglobulin. In other instances, the mammalian immunoglobulin is a murine immunoglobulin. The immunoglobulin fusion protein, immunoglobulin region and/or therapeutic peptide may further comprise one or more linkers. The linker may attach the therapeutic peptide to the immunoglobulin region. The therapeutic peptide may be a peptide or derivative or variant thereof. Alternatively, therapeutic peptide is a small molecule. The therapeutic peptide may be a growth hormone. The growth hormone may be a human growth hormone (hGH).

### Pharmacological Properties

Further disclosed herein are methods of improving one or more pharmacological properties of a therapeutic peptide. The method may comprise producing an immunoglobulin fusion protein disclosed herein. Examples of pharmacological properties may include, but are not limited to, half-life, stability, solubility, immunogenicity, toxicity, bioavailability, absorption, liberation, distribution, metabolization, and excretion. Liberation may refer to the process of releasing of a therapeutic peptide from the pharmaceutical formulation. Absorption may refer to the process of a substance entering the blood circulation. Distribution may refer to the dispersion or dissemination of substances throughout the fluids and tissues of the body. Metabolization (or biotransformation, or inactivation) may refer to the recognition by an organism that a foreign substance is present and the irreversible transformation of parent compounds into daughter metabolites. Excretion may refer to the removal of the substances from the body.

The half-life of a therapeutic peptide may greater than the half-life of the non-conjugated therapeutic peptide. The half-life of the therapeutic peptide may be greater than 4 hours, greater than 6 hours, greater than 12 hours, greater than 24 hours, greater than 36 hours, greater than 2 days, greater than 3 days, greater than 4 days, greater than 5 days, greater than 6 days, greater than 7 days, greater than 8 days, greater than 9 days, greater than 10 days, greater than 11 days, greater than 12 days, greater than 13 days, or greater than 14 days when administered to a subject. The half-life of the therapeutic peptide may be greater than 4 hours when administered to a subject. The half-life of the therapeutic peptide may be greater than 6 hours when administered to a subject.

The half-life of the therapeutic peptide may increase by at least about 2, 4, 6, 8, 10, 12, 14, 16, 18, or 20 or more hours. The half-life of the therapeutic peptide may increase by at least about 2 hours. The half-life of the therapeutic peptide may increase by at least about 4 hours. The half-life of the therapeutic peptide may increase by at least about 6 hours. The half-life of the therapeutic peptide may increase by at least about 8 hours.

The half-life of a therapeutic peptide may be at least about 1.5, 2, 2.5, 3, 3.5, 4, 4.5, 5, 5.5, 6, 6.5, 7, 7.5, 8, 8.5, 9, 9.5, or 10-fold greater than the half-life of the non-conjugated therapeutic peptide. The half-life of a therapeutic peptide an immunoglobulin described herein may be at least about 15, 16, 17, 18, 19, 20, 25, 30, 35, 40, 45, or 50-fold greater than the half-life of the non-conjugated therapeutic peptide. The half-life of a therapeutic peptide an immunoglobulin described herein may be at least about 2-fold greater than the half-life of the non-conjugated therapeutic peptide. The half-life of a therapeutic peptide an immunoglobulin described herein may be at least about 5-fold greater than the half-life of the non-conjugated therapeutic peptide. The half-life of a therapeutic peptide an immunoglobulin described herein may be at least about 10-fold greater than the half-life of the non-conjugated therapeutic peptide.

The half-life of a therapeutic peptide an immunoglobulin described herein may be at least about 5%, 10%, 15%, 20%, 25%, 30%, 35%, 40%, 45%, 55%, 60%, 65%, 70%, 75%, 80%, 85%, 90%, 95%, or 97% greater than the half-life of the non-conjugated therapeutic peptide. The half-life of a therapeutic peptide an immunoglobulin described herein may be at least about 10% greater than the half-life of the non-conjugated therapeutic peptide. The half-life of a therapeutic peptide an immunoglobulin described herein may be at least about 20% greater than the half-life of the non-conjugated therapeutic peptide. The half-life of a therapeutic peptide an immunoglobulin described herein may be at least about 30% greater than the half-life of the non-conjugated therapeutic peptide. The half-life of a therapeutic peptide an immunoglobulin described herein may be at least about 40% greater than the half-life of the non-conjugated therapeutic peptide. The half-life of a therapeutic peptide an immunoglobulin described herein may be at least about 50% greater than the half-life of the non-conjugated therapeutic peptide.

### Examples

The activity data provided in the following examples are generally obtained using the immunoglobulin fusion proteins defined in the example and exemplified by the provided SEQ ID. It is to be understood that the activities of any immunoglobulin fusion protein disclosed herein may be enhanced or attenuated depending on conditions not relating to immunoglobulin fusion protein sequence, for example, expression and purification conditions.

### Example 1: Construction of a Trastuzumab-Exendin-4 Fusion Protein Vector for Expression in Mammalian Cells

The exendin-4 (EX4) gene was synthesized by IDT (IA, USA), and amplified by polymerase chain reaction (PCR). The exendin-4 gene (SEQ ID NO: 75) was genetically fused to the nucleic acids encoding for a trastuzumab light chain (SEQ ID NO: 1) using a linker encoding for the amino acid sequence GGGGS (SEQ ID NO: 115) by overlap PCR. The pTrastuzumab(NL)-EX4 mammalian expression vector encoding for trastuzumab-EX4 light chain was created by in-frame ligation of the amplified trastuzumab-EX4 fusion (SEQ ID NO: 9) to the pFuse backbone vector (InvivoGen, CA). The gene encoding for trastuzumab heavy chain (SEQ ID NO: 2) was amplified and cloned into the pFuse vector to create a pTrastuzumab(H) mammalian expression vector. The resulting mammalian expression vectors were verified by DNA sequencing.

### Example 2: Expression and Purification of Trastuzumab-Exendin-4 Fusion Protein

A trastuzumab-EX4 fusion protein was expressed through co-transfection of freestyle HEK293 cells with vectors encoding trastuzumab(NL)-EX4 and trastuzumab(H). The cells were grown in shaker flasks at 125 rpm with freestyle 293 expression medium (Life Technologies) at 37° C. with 5% CO2. Expressed proteins were secreted into the culture medium and harvested twice every 48 hours after transfection. The fusion proteins were purified by Protein A/G chromatography (Thermo Fisher Scientific, IL) and analyzed by SDS-PAGE gel.

### Example 3: Activity of Trastuzumab Fusion Proteins to Activate GLP-1 Receptor

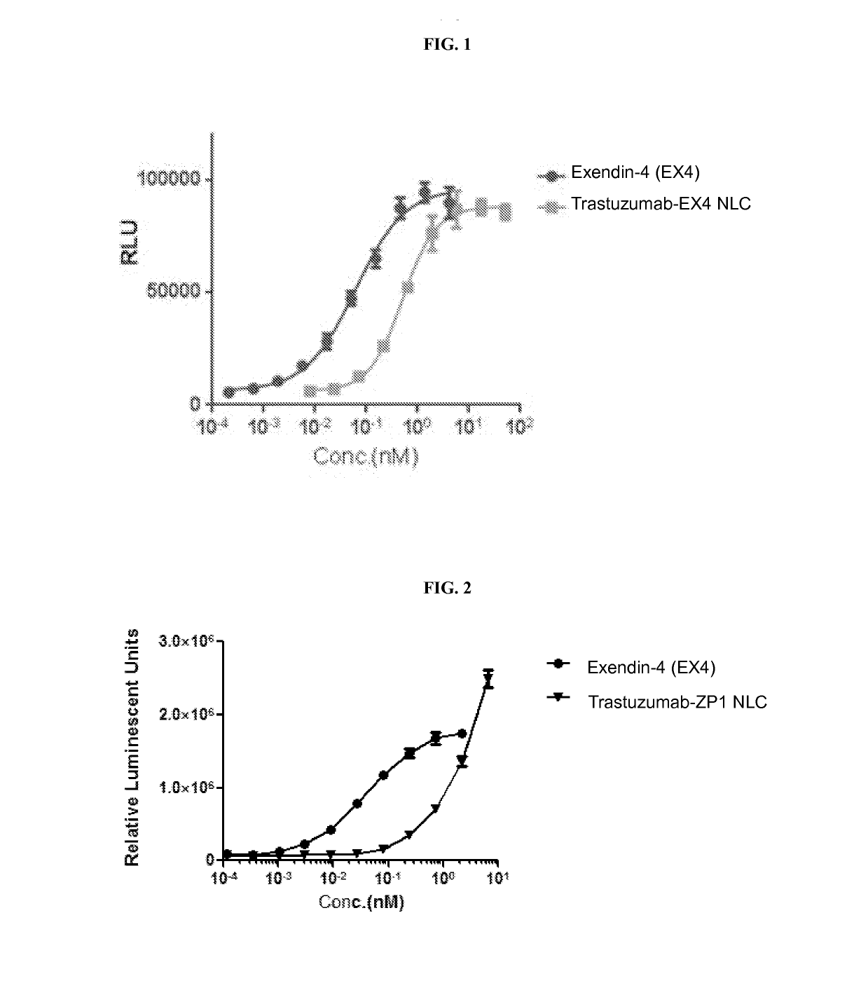

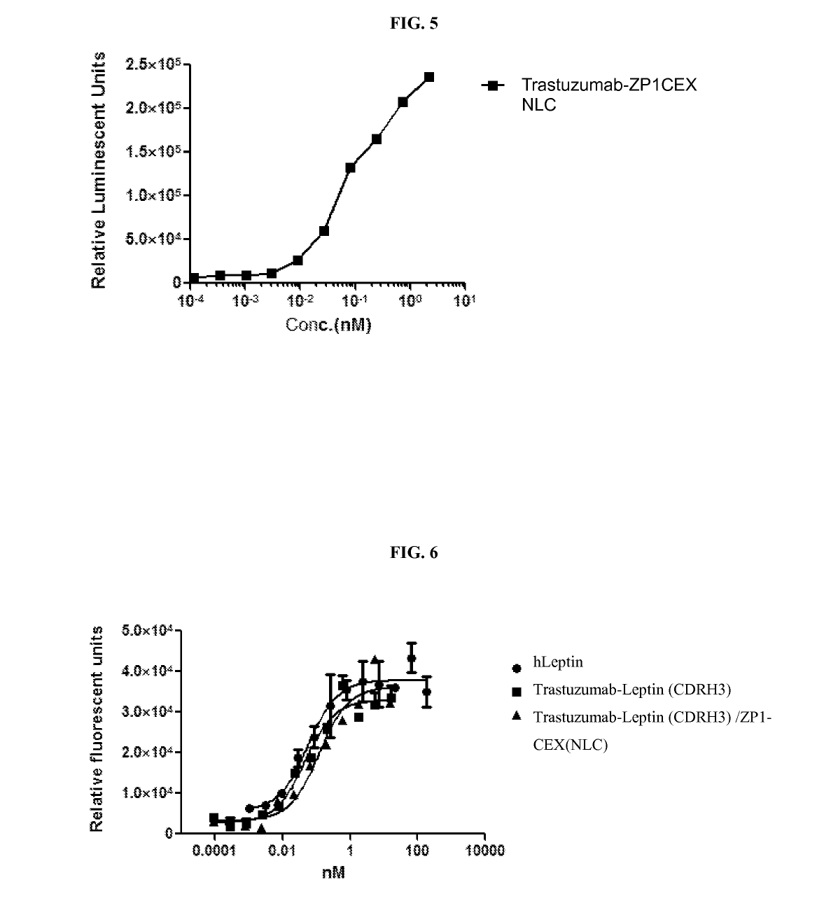

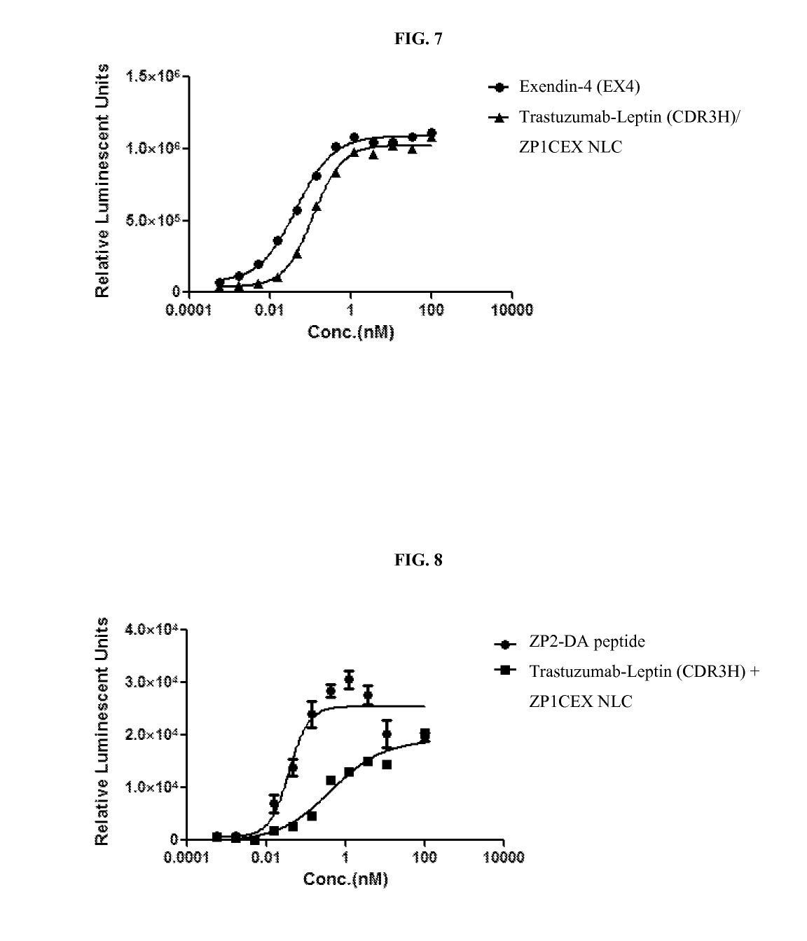

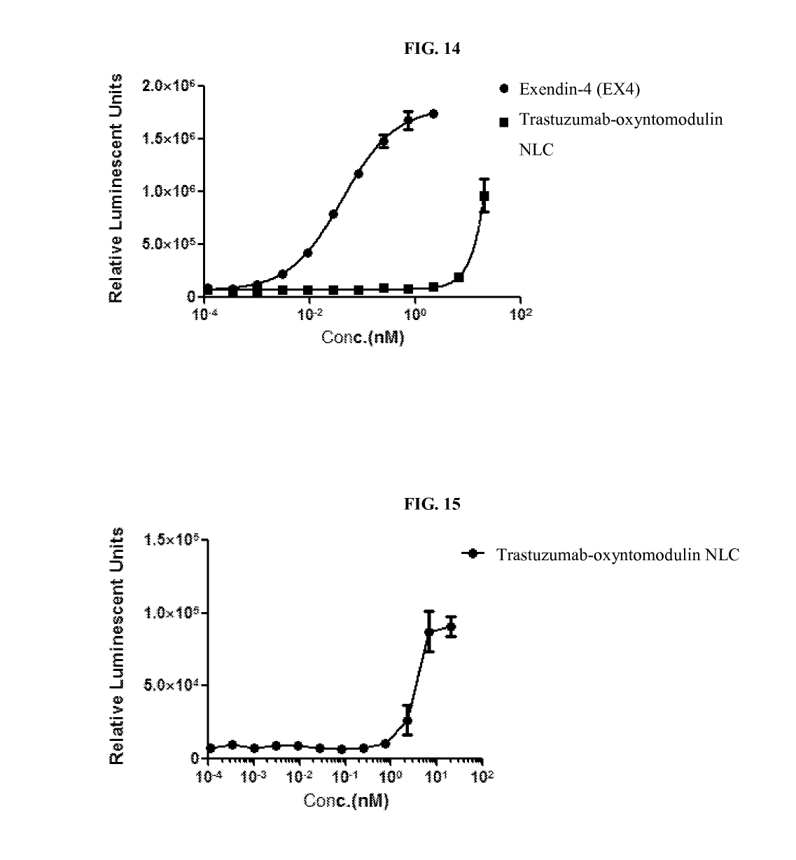

The activity of trastuzumab fusion proteins for GLP-1 receptor activation was examined by a luciferase assay. HEK293 cells expressing surface GLP-1 receptor (GLP-1R) and cAMP responsive element (CRE)-luciferase (Luc) reporter gene were grown in DMEM supplemented with 10% FBS at 37° C. with 5% CO2. Cells were seeded in 384-well plates at a density of 5,000 cells per well and treated with various concentrations of EX4 peptide, leptin, trastuzumab, trastuzumab(NL, GGGGS)-ZP1 (SEQ ID NO: 45) with trastuzumab(H) (SEQ ID NO: 6), trastuzumab(NL, GGGGS)-ZPCEX (SEQ ID NO: 46) with trastuzumab(H) (SEQ ID NO: 6), trastuzumab(CDR3H) Leptin (SEQ ID NO: 44) with trastuzumab(NL, GGGGS)-ZPCEX (SEQ ID NO: 46), trastuzumab(NL, GGGGS)-oxyntomodulin (SEQ ID NO: 68) with trastuzumab(H) (SEQ ID NO: 6), and trastuzumab-EX4 fusion for 24 hours at 37° C. with 5% CO2. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. The plots are shown in FIG. 1: EX4 EC₅₀=61 pM, trastuzumab-EX4 EC₅₀=551.3 pM; FIG. 2: EX4 EC₅₀=41.41±2.1 pM, trastuzumab(NL, GGGGS)-ZP1 (SEQ ID NO: 45) with trastuzumab(H) (SEQ ID NO: 6); FIG. 4: EX4 EC₅₀=41.41±2.1 pM, trastuzumab(NL, GGGGS)-ZPCEX (SEQ ID NO: 46) with trastuzumab(H) (SEQ ID NO: 6) EC₅₀=38.6±2.19 pM; FIG. 6: leptin EC₅₀=55.02±13.62 pM, trastuzumab(CDR3H) Leptin (SEQ ID NO: 44) EC₅₀=44.84±8.89 pM, trastuzumab(CDR3H) Leptin (SEQ ID NO: 44) with trastuzumab(NL, GGGGS)-ZPCEX (SEQ ID NO: 46) EC₅₀=117±28.51 pM; FIG. 7: EX4 EC₅₀=43.25±2.92 pM, trastuzumab(CDR3H) Leptin (SEQ ID NO: 44) with trastuzumab(NL, GGGGS)-ZPCEX (SEQ ID NO: 46) EC₅₀=114.6±5.36 pM; and FIG. 14: trastuzumab(NL, GGGGS)-oxyntomodulin (SEQ ID NO: 68) with trastuzumab(H) (SEQ ID NO: 6).

### Example 4: Activity of Trastuzumab-Based and Palivizumab-Based Fusion Proteins to Activate Glucagon Receptors

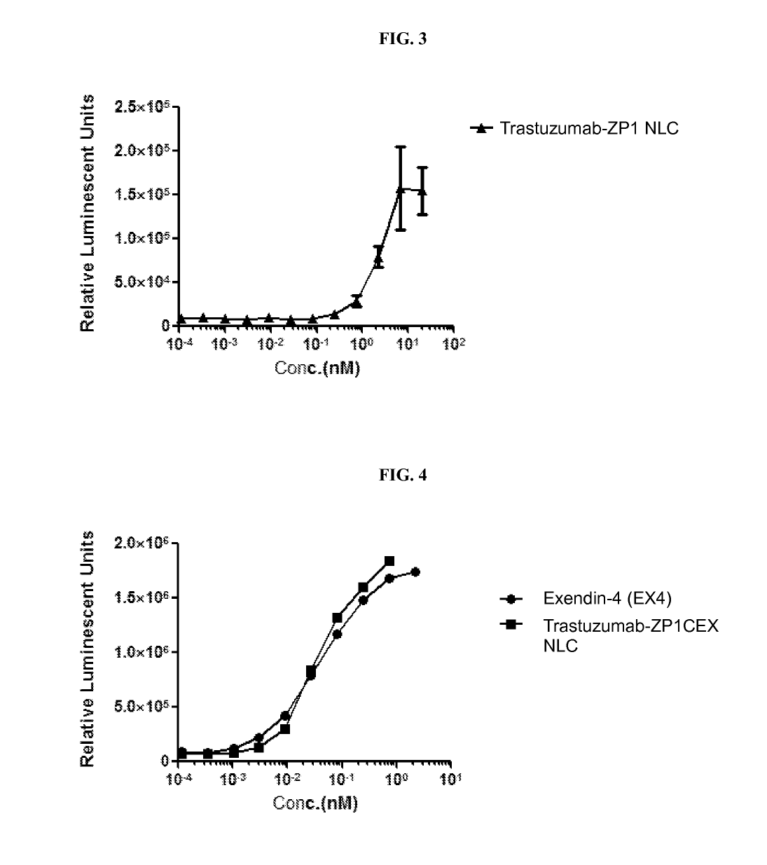

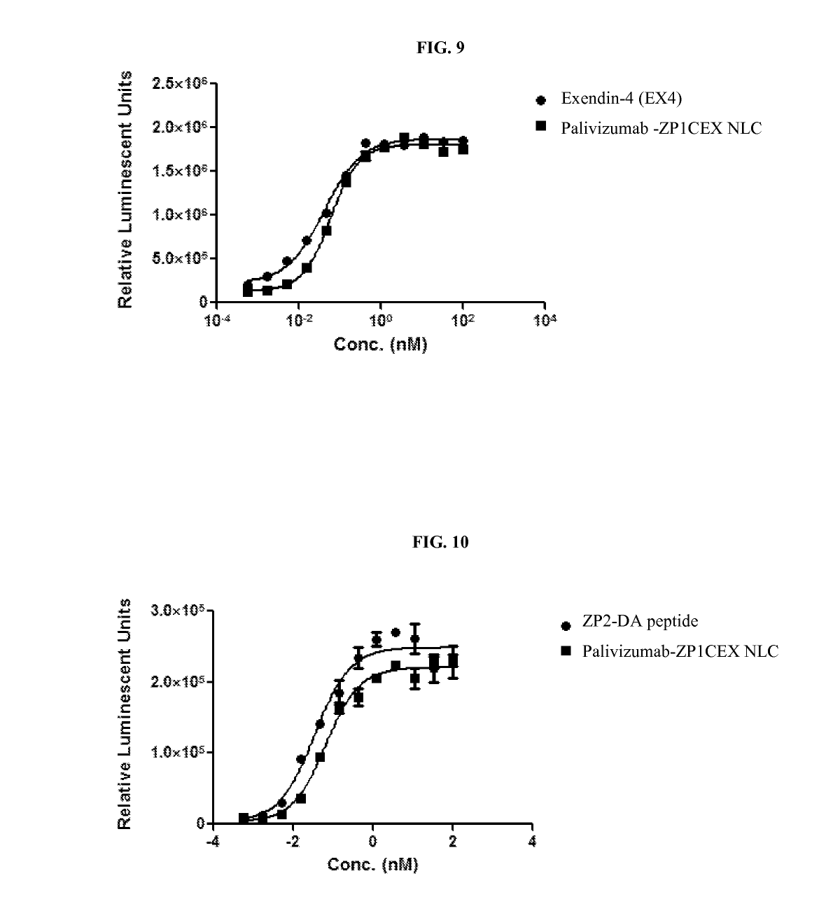

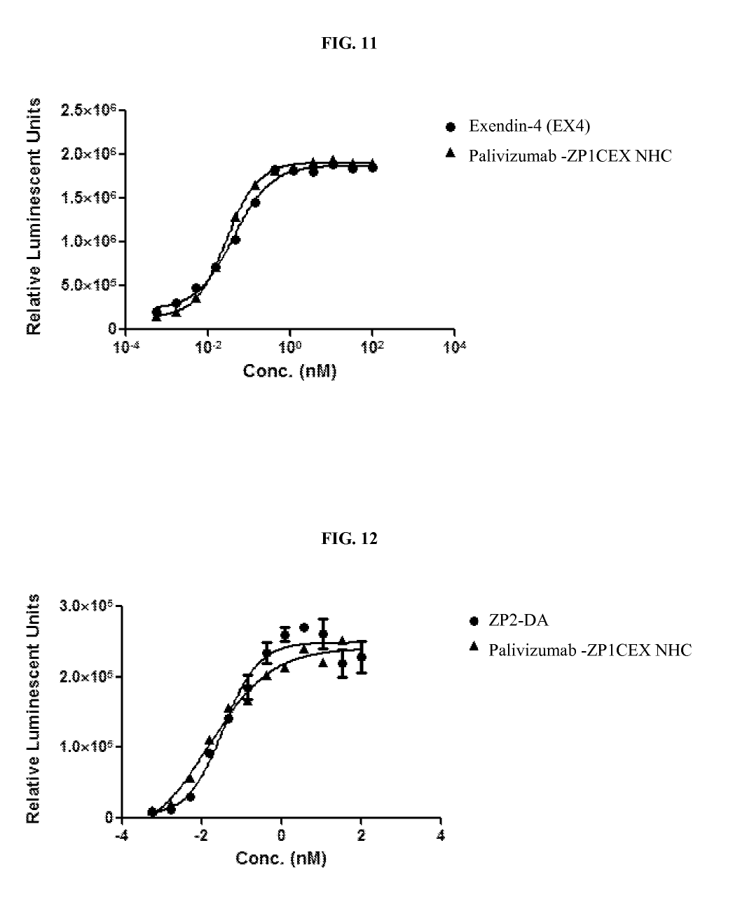

The activities of trastuzumab and palivizumab comprising fusion proteins were examined by a luciferase assay. HEK293 cells expressing surface glucagon receptor (GCGR) and cAMP responsive element (CRE)-luciferase (Luc) reporter gene were grown in DMEM supplemented with 10% FBS at 37° C. with 5% CO2. Cells were seeded in 384-well plates at a density of 5,000 cells per well and treated with various concentrations of glucagon, trastuzumab(NL)-ZP1, ZP2-DA (HsQGTFTSDY SKYLDECAAK EFICWLLRA, where s is a D-serine) (SEQ ID NO: 268), trastuzumab(NL,GGGGS)-ZP1CEX (SEQ ID NO: 46) and trastuzumab(CDR3H)-leptin, palivizumab(NL,GGGGS)-ZP1CEX (SEQ ID NO: 48), palivizumab(NH,GGGGS)-ZP1CEX (SEQ ID NO: 50), palivizumab (NL, GGGGS)-ZPCEX (SEQ ID NO: 48), palivizumab (NH, GGGGS)-ZPCEX (SEQ ID NO: 50) and trastuzumab(NL)-oxyntomodulin (SEQ ID NO: 68) proteins for 24 hours at 37° C. with 5% CO2. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. The plots are shown in FIG. 3 (trastuzumab (NL)-ZP1: EC₅₀=2.283±0.294 nM), FIG. 5 (trastuzumab (NL)-ZP1CEX: EC₅₀=92.16±14.35 pM), FIG. 8 (trastuzumab (NL)-ZP1CEX and trastuzumab (CDR)-leptin: EC₅₀=410.3±106.77 pM; ZP2-DA: EC₅₀=36.81±7.45 pM), FIG. 10 (palivizumab (NL)-ZP1CEX: EC₅₀=63.5±7.84 pM; ZP2-DA: EC₅₀=33.73±6.92 pM), FIG. 12 (palivizumab (NH)-ZP1CEX: EC₅₀=14.89±5.24 pM; ZP2-DA: EC₅₀=33.73±6.92 pM), FIG. 9 (EX4: EC₅₀=40.5±3.24 pM; palivizumab (NL, GGGGS)-ZPCEX: EC₅₀=58.77±8.14 pM), FIG. 11 (EX4: EC₅₀=40.5±3.24 pM; palivizumab (NH, GGGGS)-ZPCEX (SEQ ID NO: 50): EC₅₀=27.42±1.75 pM), and FIG. 15 (trastuzumab (NL)-oxyntomodulin).

### Example 5: Activity of palivizumab-relaxin fusion proteins to activate relaxin receptors

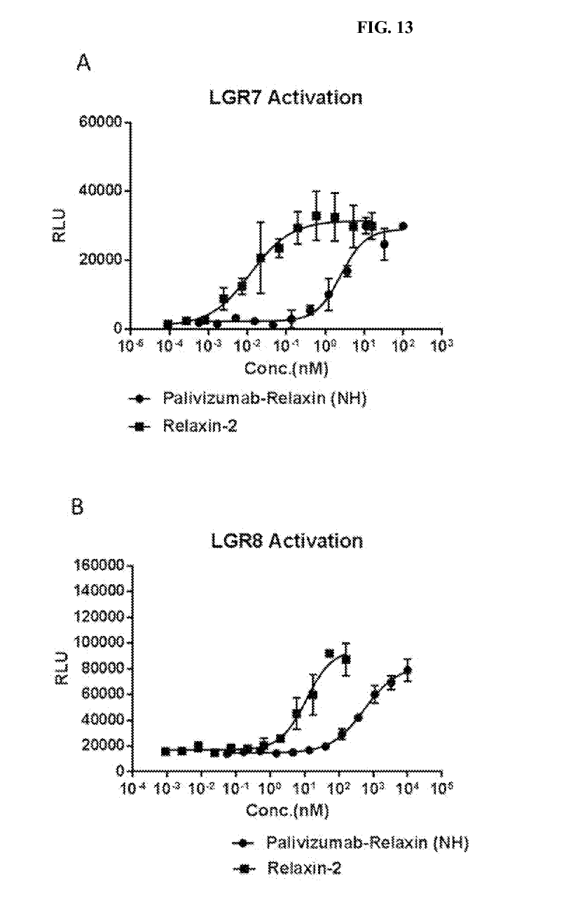

The activities of palivizumab(NH, CEXGGGGS)-relaxin2(single) (SEQ ID NO: 201) were examined by a luciferase assay. HEK293 cells overexpressed with relaxin receptor (LGR7) or (LGR8), and cAMP responsive element (CRE)-luciferase (Luc) reporter gene were grown in DMEM supplemented with 10% FBS at 37° C. with 5% CO2. Cells were seeded in 384-well plates for 24 hours and subsequently treated with various concentrations of relaxin-2 and palivizumab(NH, CEXGGGGS)-relaxin2(single) fusion protein (SEQ ID NO: 201) for an additional 24 hours. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. The plots are shown in FIGS. 13A and 13B. For LGR7 expressing cells, the EC₅₀ for relaxin-2 was 0.012 nM and the EC₅₀ for palivizumab(NH, CEXGGGGS)-relaxin2(single) was 2.5 nM. For LGR8 expressing cells, the EC₅₀ for relaxin-2 was 11.2 nM and the EC₅₀ for palivizumab(NH, CEXGGGGS)-relaxin2(single) was 552.7 nM. These data illustrate that the amino-terminal relaxin fusion proteins are comparable in their selectivity for relaxin receptors as wild-type relaxin.

### Example 6: Construction of Palivizumab-Relaxin Fusion Protein Vectors for Expression in Mammalian Cells

Relaxin nucleic acid sequences were synthesized by IDT (IA, USA), and amplified by polymerase chain reaction (PCR).

The relaxin2 (GGGPRR) (SEQ ID NO: 227) was genetically fused to nucleic acids encoding for a palivizumab heavy chain (SEQ ID NO: 4) using a connecting nucleic acid sequence encoding for the connecting peptide GGGGG (SEQ ID NO: 116) by overlap PCR to generate palivizumab(NH, GGGGG)-relaxin2(GGGPRR) (SEQ ID NO: 180). The pPalivizumab(NH, GGGGG)-relaxin2(GGGPRR) mammalian expression vector encoding for palivizumab(NH, GGGGG)-relaxin2(GGGPRR) was created by in-frame ligation of the amplified palivizumab(NH, GGGGG)-relaxin2(GGGPRR) to the pFuse backbone vector (InvivoGen, CA).

The relaxin2 (GGGPRR) (SEQ ID NO: 227) was genetically fused to nucleic acids encoding for a palivizumab heavy chain (SEQ ID NO: 4) using a connecting nucleic acid sequence encoding for the connecting peptide CEXGGGGG (SEQ ID NO: 118) by overlap PCR to generate palivizumab(NH, CEXGGGGG)-relaxin2(GGGPRR) (SEQ ID NO: 181). The pPalivizumab(NH, CEXGGGGG)-relaxin2(GGGPRR) mammalian expression vector encoding for palivizumab(NH, CEXGGGGG)-relaxin2(GGGPRR) was created by in-frame ligation of the amplified palivizumab(NH, CEXGGGGG)-relaxin2(GGGPRR) to the pFuse backbone vector (InvivoGen, CA).

The relaxin2 (GGGPRR) (SEQ ID NO: 227) was genetically fused to nucleic acids encoding for a palivizumab heavy chain (SEQ ID NO: 4) using a connecting nucleic acid sequence encoding for the connecting peptide EAAAK (SEQ ID NO: 237) by overlap PCR to generate palivizumab(NH, EAAAK)-relaxin2(GGGPRR) (SEQ ID NO: 182). The pPalivizumab(NH, EAAAK)-relaxin2(GGGPRR) mammalian expression vector encoding for palivizumab(NH, EAAAK)-relaxin2(GGGPRR) was created by in-frame ligation of the amplified palivizumab(NH, EAAAK)-relaxin2(GGGPRR) to the pFuse backbone vector (InvivoGen, CA).

The relaxin2 (single) (SEQ ID NO: 82) was genetically fused to nucleic acids encoding for a palivizumab heavy chain (SEQ ID NO: 4) using a connecting nucleic acid sequence encoding for the connecting peptide CEXGGGGS (SEQ ID NO: 238) by overlap PCR to generate palivizumab(NH, CEXGGGGS)-relaxin2(single) (SEQ ID NO: 170). The pPalivizumab(NH, CEXGGGGS)-relaxin2(single) mammalian expression vector encoding for palivizumab(NH, CEXGGGGS)-relaxin2(single) was created by in-frame ligation of the amplified palivizumab(NH, CEXGGGGS)-relaxin2(single) to the pFuse backbone vector (InvivoGen, CA).

The relaxin2 (30GS) (SEQ ID NO: 223) was genetically fused to nucleic acids encoding for a palivizumab heavy chain (SEQ ID NO: 4) using a connecting nucleic acid sequence encoding for the connecting peptide CEXGGGGG (SEQ ID NO: 118) by overlap PCR to generate palivizumab(NH, CEXGGGGG)-relaxin2(30GS) (SEQ ID NO: 173). The pPalivizumab(NH, CEXGGGGG)-relaxin2(30GS) mammalian expression vector encoding for palivizumab(NH, CEXGGGGG)-relaxin2(30GS) was created by in-frame ligation of the amplified palivizumab(NH, CEXGGGGG)-relaxin2(30GS) to the pFuse backbone vector (InvivoGen, CA).

The relaxin2 (single) (SEQ ID NO: 82) was genetically fused to nucleic acids encoding for a palivizumab heavy chain fab (portion of SEQ ID NO: 4) using a connecting nucleic acid sequence encoding for the connecting peptide CEXGGGGS (SEQ ID NO: 238) by overlap PCR to generate palivizumab fab(NH, CEXGGGGS)-relaxin2(single) (SEQ ID NO: 172). The pPalivizumab fab(NH, CEXGGGGS)-relaxin2(single) mammalian expression vector encoding for palivizumab fab(NH, CEXGGGGS)-relaxin2(single) was created by in-frame ligation of the amplified palivizumab fab(NH, CEXGGGGS)-relaxin2(single) to the pFuse backbone vector (InvivoGen, CA).

The relaxin2c (9GS) (SEQ ID NO: 226) was genetically fused to nucleic acids encoding for a palivizumab heavy chain fab (portion of SEQ ID NO: 4) using a connecting nucleic acid sequence encoding for the connecting peptide GGGGS₃ (SEQ ID NO: 115) by overlap PCR to generate palivizumab fab(NH, GGGGS₃)-relaxin2c(9GS) (SEQ ID NO: 178). The pPalivizumab fab(NH, GGGGS₃)-relaxin2(9GS) mammalian expression vector encoding for palivizumab fab(NH, GGGGS₃)-relaxin2(9GS) was created by in-frame ligation of the amplified palivizumab fab(NH, GGGGS₃)-relaxin2(9GS) to the pFuse backbone vector (InvivoGen, CA).

The relaxin2c (9GS) (SEQ ID NO: 226) was genetically fused to nucleic acids encoding for a palivizumab heavy chain (SEQ ID NO: 4) using a connecting nucleic acid sequence encoding for the connecting peptide GGGGS₃ (SEQ ID NO: 115) by overlap PCR to generate palivizumab (NH, GGGGS₃)-relaxin2c(9GS) (SEQ ID NO: 176). The pPalivizumab (NH, GGGGS₃)-relaxin2(9GS) mammalian expression vector encoding for palivizumab (NH, GGGGS₃)-relaxin2(9GS) was created by in-frame ligation of the amplified palivizumab (NH, GGGGS₃)-relaxin2(9GS) to the pFuse backbone vector (InvivoGen, CA).

The relaxin2c (9GS) (SEQ ID NO: 226) was genetically fused to nucleic acids encoding for a palivizumab heavy chain (SEQ ID NO: 4) using a connecting nucleic acid sequence encoding for the connecting peptide CEXGGGGG (SEQ ID NO: 118) by overlap PCR to generate palivizumab (NH, CEXGGGGG)-relaxin2c(9GS) (SEQ ID NO: 175). The pPalivizumab (NH, CEXGGGGG)-relaxin2(9GS) mammalian expression vector encoding for palivizumab (NH, CEXGGGGG)-relaxin2(9GS) was created by in-frame ligation of the amplified palivizumab (NH, CEXGGGGG)-relaxin2(9GS) to the pFuse backbone vector (InvivoGen, CA).

The relaxin2 (18GS) (SEQ ID NO: 228) was genetically fused to nucleic acids encoding for a palivizumab heavy chain (SEQ ID NO: 4) using a connecting nucleic acid sequence encoding for the connecting peptide GGGGS₃ (SEQ ID NO: 115) by overlap PCR to generate palivizumab (NH, GGGGS₃)-relaxin2(18GS) (SEQ ID NO: 179). The pPalivizumab (NH, GGGGS₃)-relaxin(18GS) mammalian expression vector encoding for palivizumab (NH, GGGGS₃)-relaxin(18GS) was created by in-frame ligation of the amplified palivizumab (NH, GGGGS₃)-relaxin(18GS) to the pFuse backbone vector (InvivoGen, CA).

The relaxin2 (single) (SEQ ID NO: 82) was genetically fused to nucleic acids encoding for a palivizumab heavy chain (SEQ ID NO: 4) using a connecting nucleic acid sequence encoding for the connecting peptide EAAAK (SEQ ID NO: 237) by overlap PCR to generate palivizumab(NH, EAAAK)-relaxin2(single) (SEQ ID NO: 266). The pPalivizumab(NH, EAAAK)-relaxin2(single) mammalian expression vector encoding for palivizumab(NH, EAAAK)-relaxin2(single) was created by in-frame ligation of the amplified palivizumab(NH, EAAAK)-relaxin2(single) to the pFuse backbone vector (InvivoGen, CA).

The gene encoding for palivizumab light chain (SEQ ID NO: 3) was amplified and closed into the pFuse vector to generate a pPalivizumab(L) mammalian expression vector. The resulting mammalian expression vectors were verified by DNA sequencing.

### Example 7: Expression and Purification of Palivizumab-Relaxin Fusion Proteins

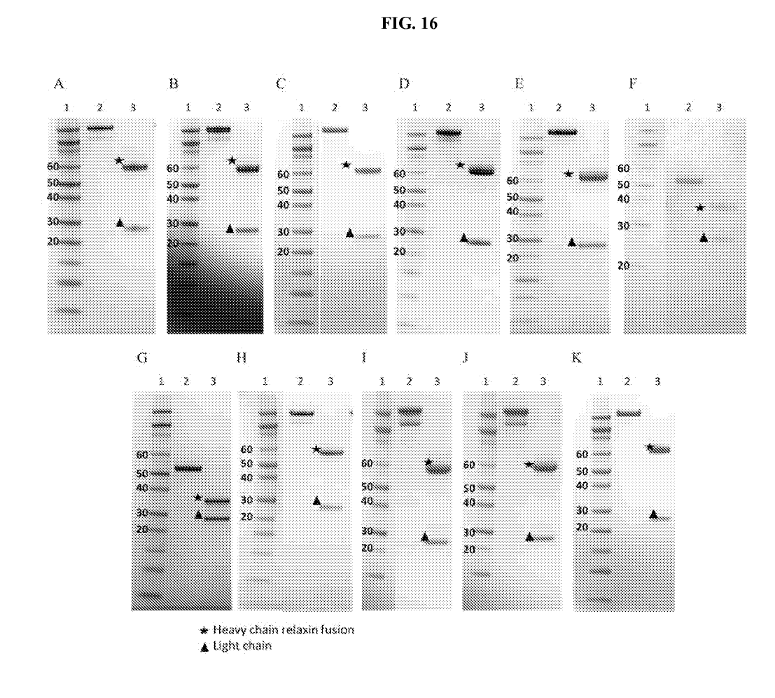

Palivizumab-relaxin heavy chain fusion proteins were each expressed through co-transfection of freestyle HEK293 cells with palivizumab-relaxin heavy chain mammalian expression vectors described in Example 7 and a palivizumab light chain mammalian expression vector. The cells were grown in shaker flasks at 125 rpm with freestyle 293 expression medium (Life Technologies) at 37° C. with 5% CO2. Expressed proteins were secreted into the culture medium and harvested twice every 48 hours after transfection. The fusion proteins were purified by Protein A/G chromatography (Thermo Fisher Scientific, IL) and analyzed by SDS-PAGE gel. Purified heavy chain fusion proteins expressed with palivizumab light chain are shown in the SDS-PAGE gels of FIG. 16. For each gel, the first lane corresponds to a molecular marker, the second lane corresponds to purified protein, and the third lane corresponds to purified protein treated with the reducing agent DTT. The heavy chains are indicated by a star. The light chains are indicated by a triangle. FIG. 16A shows purified palivizumab(NH, GGGGG)-relaxin2(GGGPRR) (SEQ ID NO: 211). FIG. 16B shows purified palivizumab(NH, CEXGGGGG)-relaxin2(GGGPRR) (SEQ ID NO: 212). FIG. 16C shows purified palivizumab(NH, EAAAK)-relaxin2(GGGPRR) (SEQ ID NO: 213). FIG. 16D shows purified palivizumab(NH, CEXGGGGS)-relaxin2(single) (SEQ ID NO: 201). FIG. 16E shows purified palivizumab(NH, CEXGGGGG)-relaxin2(30GS) (SEQ ID NO: 204). FIG. 16F shows purified palivizumab fab(NH, CEXGGGGS)-relaxin2(single) (SEQ ID NO: 203). FIG. 16G shows purified palivizumab fab(NH, GGGGS₃)-relaxin2(9GS) (SEQ ID NO: 209). FIG. 16H shows purified palivizumab (NH, GGGGS₃)-relaxin2(9GS) (SEQ ID NO: 207). FIG. 16I shows purified palivizumab (NH, CEXGGGGG)-relaxin2(9GS) (SEQ ID NO: 206). FIG. 16J shows purified palivizumab (NH, GGGGS₃)-relaxin(18GS) (SEQ ID NO: 210). FIG. 16K shows purified palivizumab(NH, EAAAK)-relaxin2(single) (SEQ ID NO: 265).

### Example 8: Activity of Palivizumab-Relaxin Fusion Proteins to Activate Relaxin Receptors

The activities of palivizumab-relaxin fusion proteins purified in Example 8 were examined by a luciferase assay. HEK293 cells overexpressed with relaxin receptor (LGR7) and cAMP responsive element (CRE)-luciferase (Luc) reporter gene were grown in DMEM supplemented with 10% FBS at 37° C. with 5% CO2. Cells were seeded in 384-well plates for 24 hours and subsequently independently treated with various concentrations of palivizumab-relaxin fusion proteins purified from Example 7 or relaxin2 peptide for an additional 24 hours. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. The EC₅₀ for relaxin-2 was 12.1 pM.

The EC₅₀ for palivizumab(NH, CEXGGGGG)-relaxin2(GGGPRR) (SEQ ID NO: 212) and palivizumab light chain (SEQ ID NO: 7) was 2,000 pM. The EC₅₀ for palivizumab(NH, EAAAK)-relaxin2(GGGPRR) (SEQ ID NO: 213) and palivizumab light chain (SEQ ID NO: 7) was 3,400 pM. The EC₅₀ for palivizumab(NH, CEXGGGGS)-relaxin2(single) (SEQ ID NO: 201) and palivizumab light chain (SEQ ID NO: 7) was 2,500 pM. The EC₅₀ for palivizumab(NH, CEXGGGGG)-relaxin2(30GS) (SEQ ID NO: 204) and palivizumab light chain (SEQ ID NO: 7) was 208 pM. The EC₅₀ for palivizumab fab(NH, CEXGGGGS)-relaxin2(single) (SEQ ID NO: 203) and palivizumab light chain (SEQ ID NO: 7) was 47,300 pM. The EC₅₀ for palivizumab fab(NH, GGGGS₃)-relaxin2(9GS) (SEQ ID NO: 209) and palivizumab light chain (SEQ ID NO: 7) was 5,800 pM. The EC₅₀ for palivizumab (NH, GGGGS₃)-relaxin2(9GS) (SEQ ID NO: 207) and palivizumab light chain (SEQ ID NO: 7) was 240 pM. The EC₅₀ for palivizumab (NH, CEXGGGGG)-relaxin2(9GS) (SEQ ID NO: 206) and palivizumab light chain (SEQ ID NO: 7) was 480 pM. The EC₅₀ for palivizumab (NH, GGGGS₃)-relaxin(18GS) (SEQ ID NO: 210) and palivizumab light chain (SEQ ID NO: 7) was 1,300 pM. The EC₅₀ for palivizumab(NH, EAAAK)-relaxin2(single) (SEQ ID NO: 266) and palivizumab light chain (SEQ ID NO: 7) was 4,290.

### Example 9: Construction of Palivizumab-Glucagon Fusion Protein Vectors for Expression in Mammalian Cells

Glucagon nucleic acid sequences were synthesized by IDT (IA, USA), and amplified by polymerase chain reaction (PCR).

The glucagon nucleic acid sequence (SEQ ID NO: 92) was genetically fused to nucleic acids encoding for a palivizumab light chain (SEQ ID NO: 3) using a connecting nucleic acid sequence encoding for the connecting peptide EAAAK (SEQ ID NO: 237) by overlap PCR to generate palivizumab(NL, EAAAK)-glucagon (SEQ ID NO: 162). The pPalivizumab(NL, EAAAK)-glucagon mammalian expression vector encoding for palivizumab(NL, EAAAK)-glucagon was created by in-frame ligation of the amplified palivizumab(NL, EAAAK)-glucagon to the pFuse backbone vector (InvivoGen, CA).

The resulting mammalian expression vectors were verified by DNA sequencing.

### Example 10: Construction of Palivizumab-Exendin-4 Fusion Protein Vectors for Expression in Mammalian Cells

Exendin-4 nucleic acid sequences were synthesized by IDT (IA, USA), and amplified by polymerase chain reaction (PCR).

The exendin-4 nucleic acid sequence (SEQ ID NO: 75) was genetically fused to nucleic acids encoding for a palivizumab heavy chain (SEQ ID NO: 4) using a connecting nucleic acid sequence encoding for the connecting peptide GGGGS₁ (SEQ ID NO: 115) by overlap PCR to generate palivizumab(NH, GGGGS₁)-exendin-4 (SEQ ID NO: 161). The pPalivizumab(NH, GGGGS₁)-exendin-4 mammalian expression vector encoding for palivizumab(NH, GGGGS₁)-exendin-4 was created by in-frame ligation of the amplified palivizumab(NH, GGGGS₁)-exendin-4 to the pFuse backbone vector (InvivoGen, CA).

The resulting mammalian expression vectors were verified by DNA sequencing.

### Example 11: Expression and Purification of Palivizumab-Glucagon Fusion Protein and Palivizumab-Exendin-4 Fusion Protein

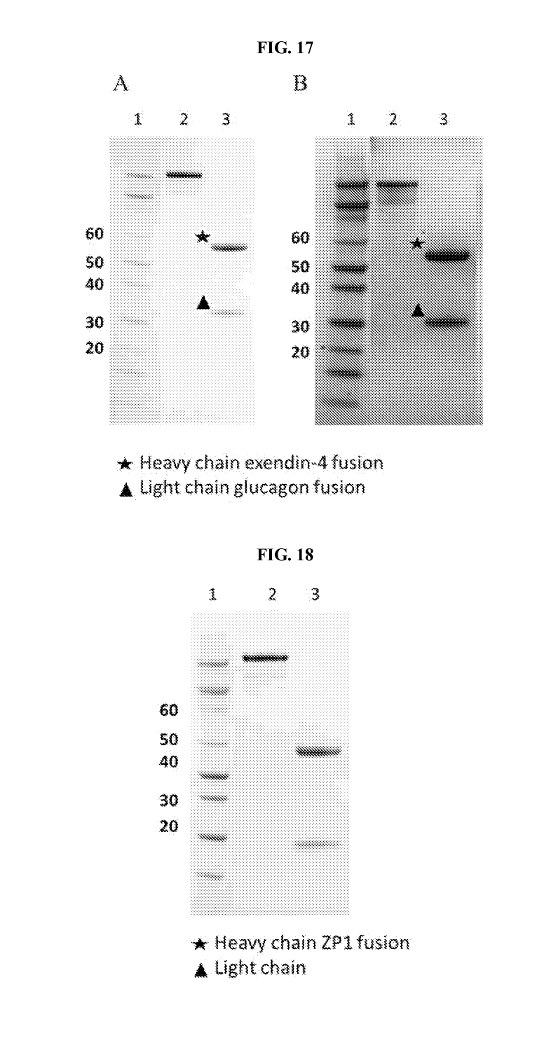

Palivizumab-glucagon light chain fusion protein and palivizumab-exendin-4 heavy chain fusion protein were co-expressed through co-transfection of freestyle HEK293 cells with pPalivizumab(NL, EAAAK)-glucagon and pPalivizumab(NH, GGGGS₁)-exendin-4 mammalian expression vectors described in Examples 10 and 11. The cells were grown in shaker flasks at 125 rpm with freestyle 293 expression medium (Life Technologies) at 37° C. with 5% CO2. Expressed proteins were secreted into the culture medium and harvested twice every 48 hours after transfection. The fusion proteins were purified by Protein A/G chromatography (Thermo Fisher Scientific, IL) and analyzed by SDS-PAGE gel. Purified fusion proteins are shown in the SDS-PAGE gels of FIG. 17. For each gel, the first lane corresponds to a molecular marker, the second lane corresponds to purified protein, and the third lane corresponds to purified protein treated with the reducing agent DTT. The heavy chains are indicated by a star. The light chains are indicated by a triangle. FIG. 17A shows purified palivizumab(NL, EAAAK)-glucagon(2S) and pPalivizumab(NH, GGGGS₁)-exendin-4. FIG. 17B shows purified palivizumab(NL, EAAAK)-glucagon(2G) and pPalivizumab(NH, GGGGS₁)-exendin-4.

### Example 12: Activity of Palivizumab Fusion Proteins Fusion Proteins to Activate Glucagon Receptors

The activities of palivizumab fusion proteins were examined by a luciferase assay. HEK293 cells expressing a surface glucagon receptor or GLP-1 receptor (GCGR or GLP-1R) and cAMP responsive element (CRE)-luciferase (Luc) reporter gene were grown in DMEM supplemented with 10% FBS at 37° C. with 5% CO2. Cells were seeded in 384-well plates at a density of 5,000 cells per well and treated with various concentrations of exendin-4, glucagon, and palivizumab-glucagon light chain and palivizumab-exendin-4 heavy chain fusion proteins (from Example 11) for 24 hours at 37° C. with 5% CO2. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. Data was analyzed using GraphPad Prism 6 software. For cells expressing GLP-1R, the EC₅₀ for exendin-4 was 57 pM. For cells expressing GLP-1R, the EC₅₀ for palivizumab(NL, EAAAK)-glucagon(2S) and pPalivizumab(NH, GGGGS₁)-exendin-4 was 13 pM. For cells expressing GLP-1R, the EC₅₀ for palivizumab(NL, EAAAK)-glucagon(2G) and pPalivizumab(NH, GGGGS₁)-exendin-4 was 9 pM. For cells expressing GCGR, the EC₅₀ for glucagon was 95 pM. For cells expressing GCGR, the EC₅₀ for palivizumab(NL, EAAAK)-glucagon(2G) and pPalivizumab(NH, GGGGS₁)-exendin-4 was 26 pM. For cells expressing GCGR, the EC₅₀ for palivizumab(NL, EAAAK)-glucagon(2S) and pPalivizumab(NH, GGGGS₁)-exendin-4 was 33 pM.

### Example 13: Construction of Palivizumab-ZP1 Fusion Protein Vectors for Expression in Mammalian Cells

ZP1 nucleic acid sequences were synthesized by IDT (IA, USA), and amplified by polymerase chain reaction (PCR).

The ZP1 nucleic acid sequence (SEQ ID NO: 77) was genetically fused to nucleic acids encoding for a palivizumab heavy chain (SEQ ID NO: 4) using a connecting nucleic acid sequence encoding for the connecting peptide EAAAK (SEQ ID NO: 237) by overlap PCR to generate palivizumab(NH, EAAAK)-ZP1 (SEQ ID NO: 165). The pPalivizumab(NH, EAAAK)-ZP1 mammalian expression vector encoding for palivizumab(NH, EAAAK)-ZP1 was created by in-frame ligation of the amplified palivizumab(NH, EAAAK)-ZP1 to the pFuse backbone vector (InvivoGen, CA).

The gene encoding for palivizumab light chain (SEQ ID NO: 3) was amplified and closed into the pFuse vector to generate a pPalivizumab(L) mammalian expression vector. The resulting mammalian expression vectors were verified by DNA sequencing.

### Example 14: Expression and Purification of Palivizumab-ZP1 Fusion Proteins

Palivizumab-ZP1 heavy chain fusion proteins were expressed through co-transfection of freestyle HEK293 cells with palivizumab-ZP1 heavy chain mammalian expression vectors (Example 14) and a palivizumab light chain mammalian expression vector. The cells were grown in shaker flasks at 125 rpm with freestyle 293 expression medium (Life Technologies) at 37° C. with 5% CO2. Expressed proteins were secreted into the culture medium and harvested twice every 48 hours after transfection. The fusion proteins were purified by Protein A/G chromatography (Thermo Fisher Scientific, IL) and analyzed by SDS-PAGE gel. Purified heavy chain fusion proteins expressed with palivizumab light chain are shown in the SDS-PAGE gel of FIG. 18. The first lane corresponds to a molecular marker, the second lane corresponds to purified protein, and the third lane corresponds to purified protein treated with the reducing agent DTT. The heavy chain is indicated by a star. The light chain is indicated by a triangle. FIG. 18 shows purified palivizumab(NH, EAAAK)-ZP1 (SEQ ID NO: 196) and palivizumab(L) (SEQ ID NO: 7).

### Example 15: Activity of Palivizumab-ZP1 Fusion Proteins to Activate Glucagon Receptors

The activities of palivizumab-ZP1 heavy chain fusion and palivizumab light chain were examined by a luciferase assay. HEK293 cells expressing a surface glucagon receptor or GLP-1 receptor (GCGR or GLP-1R) and cAMP responsive element (CRE)-luciferase (Luc) reporter gene were grown in DMEM supplemented with 10% FBS at 37° C. with 5% CO2. Cells were seeded in 384-well plates at a density of 5,000 cells per well and treated with various concentrations of exendin-4, glucagon, and palivizumab-ZP1 heavy chain and palivizumab light chain (Example 15) for 24 hours at 37° C. with 5% CO2. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. Data was analyzed using GraphPad Prism 6 software. For cells expressing GLP-1R, the EC₅₀ for exendin-4 was 17 pM. For cells expressing GLP-1R, the EC₅₀ for palivizumab(NH, EAAAK)-ZP1 (SEQ ID NO: 196) and palivizumab(L) (SEQ ID NO: 7) was 3 pM. For cells expressing GCGR, the EC₅₀ for glucagon was 95 pM. For cells expressing GCGR, the EC₅₀ for palivizumab(NH, EAAAK)-ZP1 (SEQ ID NO: 196) and palivizumab(L) (SEQ ID NO: 7) was 14 pM.

### Example 16: Construction of Palivizumab-GLP2 Fusion Protein Vectors for Expression in Mammalian Cells

GLP2 nucleic acid sequences were synthesized by IDT (IA, USA), and amplified by polymerase chain reaction (PCR).

The GLP2 nucleic acid sequence (SEQ ID NO: 87) was genetically fused to nucleic acids encoding for a palivizumab heavy chain (SEQ ID NO: 4) using a connecting nucleic acid sequence encoding for the connecting peptide EAAAK (SEQ ID NO: 237) by overlap PCR to generate palivizumab(NH, EAAAK)-GLP2 (SEQ ID NO: 189). The pPalivizumab(NH, EAAAK)-GLP2 mammalian expression vector encoding for palivizumab(NH, EAAAK)-GLP2 was created by in-frame ligation of the amplified palivizumab(NH, EAAAK)-GLP2 to the pFuse backbone vector (InvivoGen, CA).

The GLP2 nucleic acid sequence (SEQ ID NO: 87) was genetically fused to nucleic acids encoding for a palivizumab heavy chain (SEQ ID NO: 4) using a connecting nucleic acid sequence encoding for the connecting peptide CEXGGGGS (SEQ ID NO: 238) by overlap PCR to generate palivizumab(NH, CEXGGGGS)-GLP2 (SEQ ID NO: 187). The pPalivizumab(NH, CEXGGGGS)-GLP2 mammalian expression vector encoding for palivizumab(NH, CEXGGGGS)-GLP2 was created by in-frame ligation of the amplified palivizumab(NH, CEXGGGGS)-GLP2 to the pFuse backbone vector (InvivoGen, CA).

The gene encoding for palivizumab light chain (SEQ ID NO: 3) was amplified and closed into the pFuse vector to generate a pPalivizumab(L) mammalian expression vector. The resulting mammalian expression vectors were verified by DNA sequencing.

### Example 17: Expression and Purification of Palivizumab-GLP2 Fusion Proteins

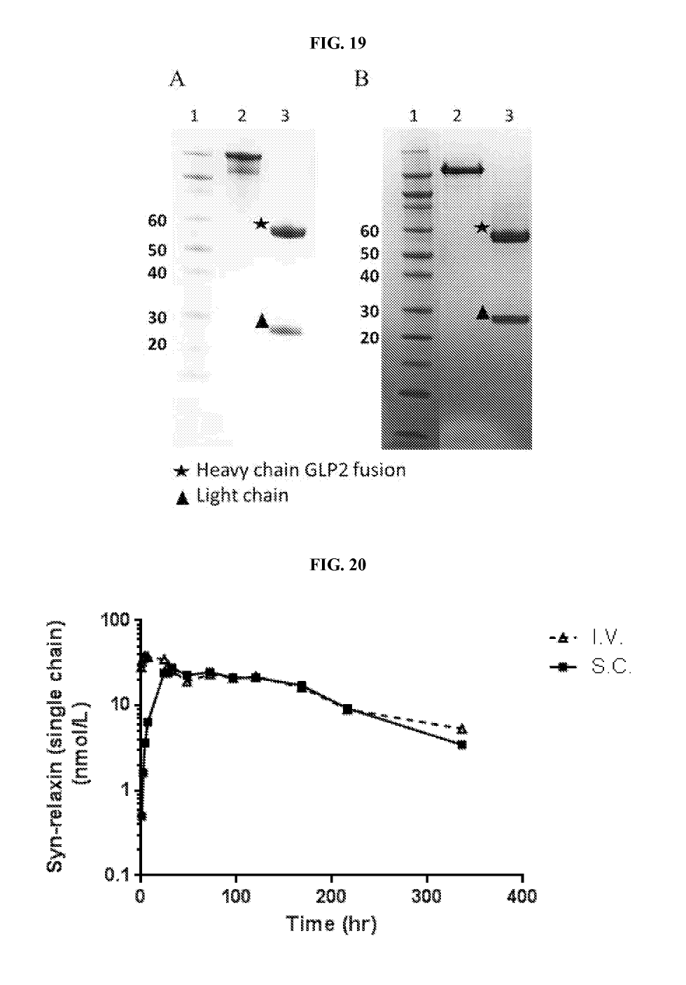

Palivizumab-GLP2 heavy chain fusion proteins were expressed through co-transfection of freestyle HEK293 cells with palivizumab-GLP2 heavy chain mammalian expression vectors (Example 17) and a palivizumab light chain mammalian expression vector. The cells were grown in shaker flasks at 125 rpm with freestyle 293 expression medium (Life Technologies) at 37° C. with 5% CO2. Expressed proteins were secreted into the culture medium and harvested twice every 48 hours after transfection. The fusion proteins were purified by Protein A/G chromatography (Thermo Fisher Scientific, IL) and analyzed by SDS-PAGE gel. Purified heavy chain fusion proteins expressed with palivizumab light chain are shown in the SDS-PAGE gels of FIG. 19. For each gel, the first lane corresponds to a molecular marker, the second lane corresponds to purified protein, and the third lane corresponds to purified protein treated with the reducing agent DTT. The heavy chains are indicated by a star. The light chains are indicated by a triangle. FIG. 19A shows purified palivizumab(NH, EAAAK)-GLP2 (SEQ ID NO: 220) and palivizumab(L) (SEQ ID NO: 7). FIG. 19B shows purified palivizumab(NH, CEXGGGGS)-GLP2 (SEQ ID NO: 218) and palivizumab(L) (SEQ ID NO: 7).

### Example 18: Activity of Palivizumab-GLP2 Fusion Proteins to Activate Glucagon Receptors

The activities of palivizumab-GLP2 heavy chain fusions and palivizumab light chain were examined by a luciferase assay. HEK293 cells expressing a surface GLP-2 receptor (GLP-2R) and cAMP responsive element (CRE)-luciferase (Luc) reporter gene were grown in DMEM supplemented with 10% FBS at 37° C. with 5% CO2. Cells were seeded in 384-well plates at a density of 5,000 cells per well and treated with various concentrations of GLP2 and palivizumab-GLP2 heavy chain and palivizumab light chain (Example 18) for 24 hours at 37° C. with 5% CO2. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. Data was analyzed using GraphPad Prism 6 software. The EC₅₀ for GLP2 was 46 pM. The EC₅₀ for palivizumab(NH, EAAAK)-GLP2 (SEQ ID NO: 220) and palivizumab(L) (SEQ ID NO: 7) was 69 pM. The EC₅₀ palivizumab(NH, CEXGGGGS)-GLP2 (SEQ ID NO: 218) and palivizumab(L) (SEQ ID NO: 7) was 133 pM.

### Example 19: Pharmacokinetic Studies of Palivizumab-Relaxin Fusion Protein

Palivizumab(NH, CEXGGGGG)-relaxin2 (single) (SEQ ID NO: 201) was injected intravenously (i.v) or subcutaneously (s.c.) into two separate experiment groups into SD female rats at doses of 2.4 nmol/kg for both modes of administration. Plasma samples were collected over the course of 350 hours. Palivizumab(NH, CEXGGGGG)-relaxin2 (single) levels were quantified using a sandwich ELISA assay. Briefly, 96 well plates were incubated with anti-hFc (abcam 98616, 1:100 dilution, PBS) at 4° C. overnight. This coating solution was poured off and the plates were blocked with blocking buffer (2% milk in 0.5% Tween-20/PBS) at room temperature for 1 hr. The blocking solution was poured off and the plates were incubated with serum dilutions (in blocking buffer) at room temperature for 2 hrs, the serum was diluted 10-10⁶ times. The serum was poured off and the plates were washed extensively by 0.5% Tween-20/PBS, and then incubated with anti-relaxin (Millipore, 553850, 1:1000 dilution, blocking buffer) at room temperature for 1 hr. The solution was poured off and the plates were washed extensively by 0.5% Tween-20/PBS, and then incubated with anti-rabbit-HRP (Life technologies, A16023, 1:3000 dilution, blocking buffer) at room temperature for 30 mins. The solution was poured of and the plates were washed extensively by 0.5% Tween-20/PBS, developed with QuantaBlu fluorogenic peroxidase substrate (Life technologies, 15169), and quantified using Spectramax fluorescence plate reader. The amount of palivizumab(NH, CEXGGGGG)-relaxin2 (single) fusion protein in plasma samples was quantified by extrapolating the signal into a linear range (signal vs concentration) of a standard curve. Pharmacokinetic parameters were modeled using WinNonlin (Pharsight). The concentrations of fusion protein at each collection time point were plotted and are shown in FIG. 20. The palivizumab(NH, CEXGGGGG)-relaxin2 (single) fusion protein had an extended half-life as compared to wild type relaxin which has a half-life of less than 0.5 hrs. The half-life of palivizumab(NH, CEXGGGGG)-relaxin2 (single) fusion protein was 79 hours for s.c. administration and 115 hours for i.v. administration. The Cₘₐₓ for the s.c. route was 27.75 nM and 38.06 nM for the i.v. route of administration. The AUC_{∞} was 5231.51 (hr*nM) for the s.c. route and 6298.81 for the i.v. route of administration.

### Example 20: Dose-Response Efficacy of a Palivizumab-Relaxin Fusion Protein in a Mouse Interpubic Ligament Model

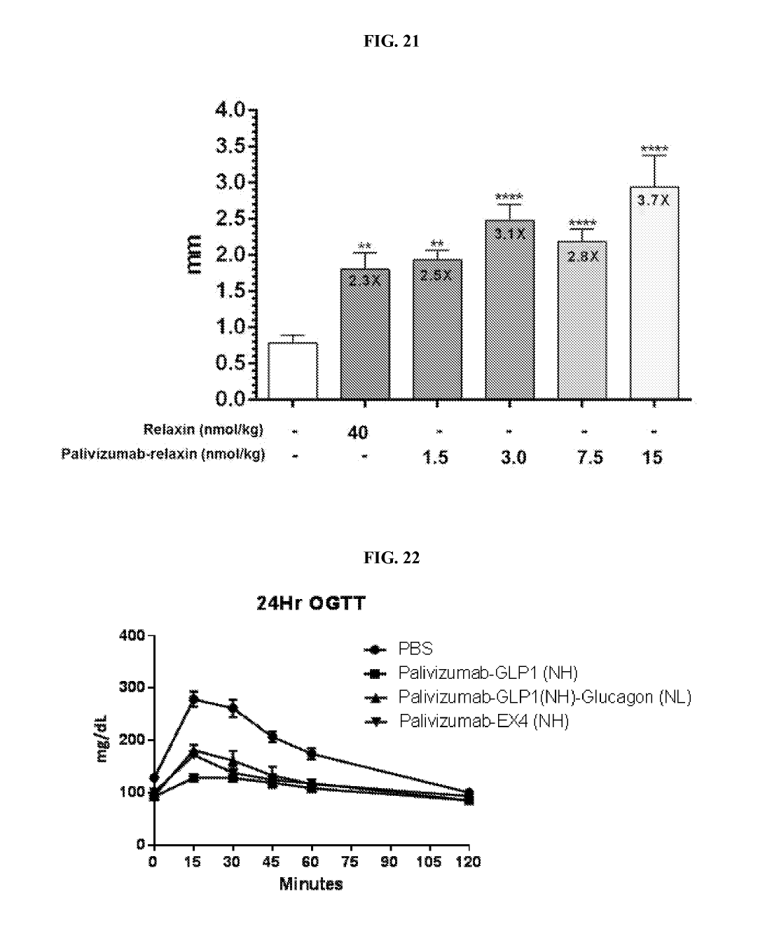

Virgin female CD1 mice weighing 18-20 g were purchased from Harlan. Mice were maintained in a temperature (23-25° C.) and light controlled room (12 h dark, 12 h bright) and were given free access to regular rodent diet and water. One week prior to treatment, mice were estrogen primed by subcutaneous injection with 5 ug estradiol cypionate in 0.1 ml sesame oil. One week after estrogen priming, the mice were treated with s.c. doses of relaxin (40 nmol/kg), palivizumab(NH, CEXGGGGG)-relaxin2 (single) fusion protein (1.5, 3.0, 7.5, 15 nmol/kg). Interpubic ligament length was measured at 24 hours after dosing using a caliper. The palivizumab(NH, CEXGGGGG)-relaxin2 (single) fusion showed a dose-response efficacy down to 1.5 nmol/kg, which had a similar efficacy as 40 nmol/kg of relaxin. FIG. 21 provides interpubic ligament lengths (mm) versus dosage of relaxin or palivizumab(NH, CEXGGGGG)-relaxin2 (single) fusion protein.

### Example 21: Pharmacodynamics of Palivizumab Fusion Proteins in Mice

Single doses of palivizumab fusion proteins (8 mg/kg) or PBS were administered by s.c. injection into CD1 mice (N=5). Glucose (3 g/kg, p.o.) was given at 30 minutes, 24, 48, 72, 96, 120, 144, 168 and 216 hours post-single dose treatments, followed by blood glucose measurements immediately prior to and at 15, 30, 45, 60, and 120 minutes post glucose load. Fusion proteins administered were: palivizumab(NH, GGGGS)-GLP1 (SEQ ID NO: 217); palivizumab(NH, GGGGS)-GLP1 (SEQ ID NO: 217) with palivizumab(NL, GGGGG)-glucagon (SEQ ID NO: 194); and palivizumab(NH, GGGGS)-exendin4 (SEQ ID NO: 192). Plots of glucose measurements for each fusion protein versus time are shown in the graph of FIG. 22.

### Example 22: Expression and Purification of Palivizumab-Relaxin (Dual) Fusion Protein

Plasmids encoding palivizumab(NH, EAAAK)-relaxin(dual) (SEQ ID NO: 222), 0.4 mg, and palivizumab(LC) (SEQ ID NO: 7), 0.2 mg, were transfected with PC2 plasmid, 0.4 mg, to 600 mL HEK 293 cells. The cultures were grown with shaking at 37° C. and then cultured at 72-96 hours. The cell cultures were centrifuged and the supernatant (600 ml) loaded onto 3 ml Protein A beads equilibrated with DPBS. The beads were washed with 25 mL DPBS and the bound protein eluted with 10 mL 0.1 M glycine, pH 2.7, which was subsequently supplemented with 1 mL of 1 M Tris-HCL, pH 8.9. Eluted proteins were applied to 3 mL of DPBS equilibrated Ni-NTA beads and the unbound fraction comprising palivizumab(NH, EAAAK)-relaxin(dual), 0.4 mg, and palivizumab(LC) was collected.

### Example 23: Activity of Palivizumab-Relaxin Fusion (Dual) Protein to Activate Relaxin Receptors

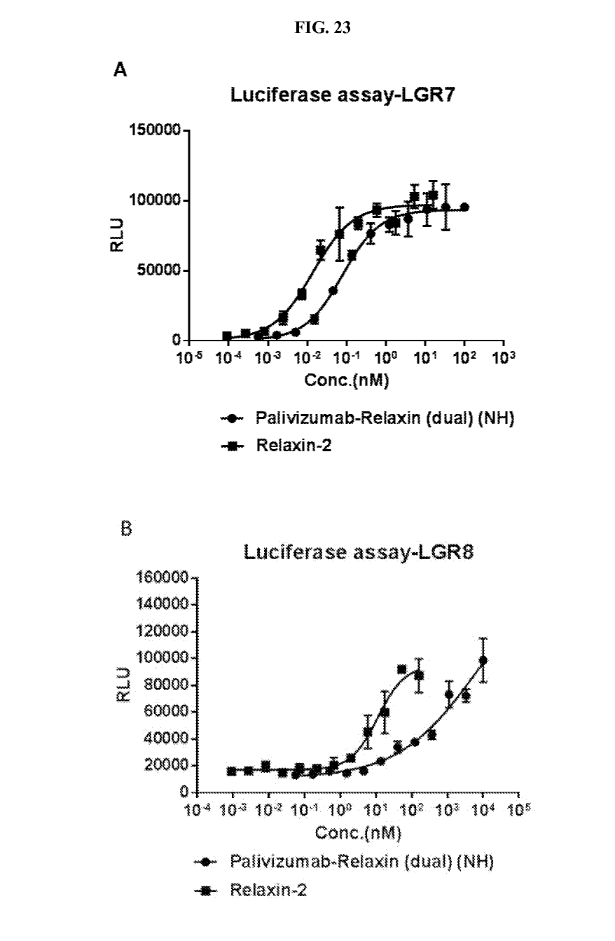

The activity of palivizumab(NH, EAAAK)-relaxin(dual) (SEQ ID NO: 222) with palivizumab(LC) (SEQ ID NO: 7), purified in Example 22, was examined by a luciferase assay. HEK293 cells overexpressed with relaxin receptor (LGR7) or (LGR8), and cAMP responsive element (CRE)-luciferase (Luc) reporter gene were grown in DMEM supplemented with 10% FBS at 37° C. with 5% CO2. Cells were seeded in 384-well plates for 24 hours and subsequently treated with various concentrations of relaxin-2 and palivizumab(NH, EAAAK)-relaxin(dual) with palivizumab(LC) for an additional 24 hours. Luminescence intensities were then measured using One-Glo (Promega) luciferase reagent by following manufacturer's instruction. The EC₅₀ values were determined by fitting data into a logistic sigmoidal function: y=A2+(A1-A2)/(1+(x/x0)p), where A1 is the initial value, A2 is the final value, x0 is the inflection point of the curve, and p is the power. The plots are shown in FIGS. 23A and 23B. For LGR7 expressing cells, the EC₅₀ for relaxin-2 was 0.014 nM and the EC₅₀ for palivizumab(NH, EAAAK)-relaxin(dual) with palivizumab(LC) was 0.079 nM. For LGR8 expressing cells, the EC₅₀ for relaxin-2 was 11.2 nM and the EC₅₀ for palivizumab(NH, EAAAK)-relaxin(dual) with palivizumab(LC) was 6766 nM. These data illustrate that the amino-terminal relaxin fusion proteins are comparable in their selectivity for relaxin receptors as wild-type relaxin.

### Example 24: Pharmacokinetic Studies of Palivizumab-Relaxin (Dual) Fusion Protein

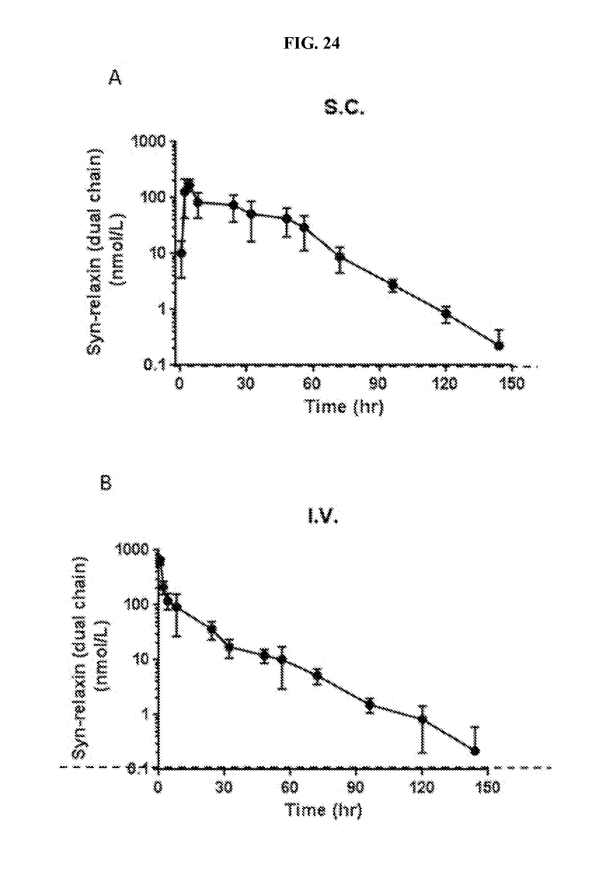

Palivizumab(NH, EAAAK)-relaxin(dual) (SEQ ID NO: 222) with palivizumab(LC) (SEQ ID NO: 7), purified in Example 22, was injected intravenously (i.v) or subcutaneously (s.c.) into two separate experiment groups into SD female rats at doses of 20 nmol/kg for both modes of administration. Plasma samples were collected over the course of 150 hours. Palivizumab(NH, EAAAK)-relaxin(dual) with palivizumab(LC) levels were quantified using a sandwich ELISA assay. Briefly, 96 well plates were incubated with anti-hFc (abcam 98616, 1:100 dilution, PBS) at 4° C. overnight. This coating solution was poured off and the plates were blocked with blocking buffer (2% milk in 0.5% Tween-20/PBS) at room temperature for 1 hr. The blocking solution was poured off and the plates were incubated with serum dilutions (in blocking buffer) at room temperature for 2 hrs, the serum was diluted 10-10⁶ times. The serum was poured off and the plates were washed extensively by 0.5% Tween-20/PBS, and then incubated with anti-relaxin (Millipore, 553850, 1:1000 dilution, blocking buffer) at room temperature for 1 hr. The solution was poured off and the plates were washed extensively by 0.5% Tween-20/PBS, and then incubated with anti-rabbit-HRP (Life technologies, A16023, 1:3000 dilution, blocking buffer) at room temperature for 30 mins. The solution was poured of and the plates were washed extensively by 0.5% Tween-20/PBS, developed with QuantaBlu fluorogenic peroxidase substrate (Life technologies, 15169), and quantified using Spectramax fluorescence plate reader. The amount of fusion protein in plasma samples was quantified by extrapolating the signal into a linear range (signal vs concentration) of a standard curve. Pharmacokinetic parameters were modeled using WinNonlin (Pharsight). The concentrations of fusion protein at each collection time point were plotted and are shown in FIGS. 24A (s.c. administration) and 24B (i.v. administration). The palivizumab(NH, EAAAK)-relaxin(dual) fusion protein had an extended half-life as compared to wild type relaxin which has a half-life of less than 0.5 hrs. The half-life of palivizumab(NH, EAAAK)-relaxin(dual) fusion protein was 14 hours for s.c. administration and 17 hours for i.v. administration. The Cₘₐₓ for the s.c. route was 170.24 nM and 660.99 nM for the i.v. route of administration. The AUC_{∞} was 4223.08 (hr*nM) for the s.c. route and 3624.51 for the i.v. route of administration.

### Example 25: Dose-Response Efficacy of a Palivizumab-Relaxin (Dual) Fusion Protein in a Mouse Interpubic Ligament Model

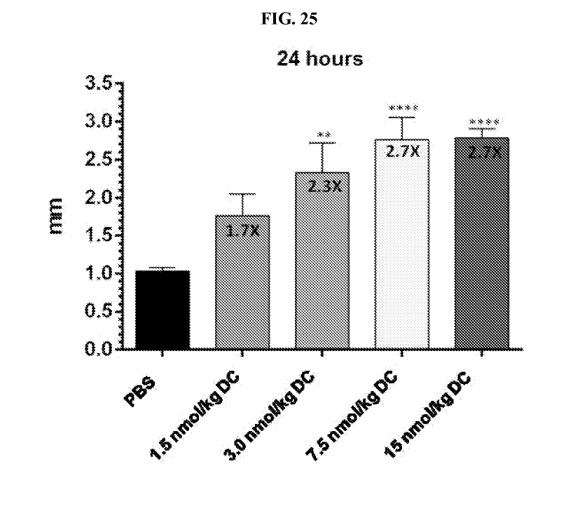

Virgin female CD1 mice weighing 18-20 g were purchased from Harlan. Mice were maintained in a temperature (23-25° C.) and light controlled room (12 h dark, 12 h bright) and were given free access to regular rodent diet and water. One week prior to treatment, mice were estrogen primed by subcutaneous injection with 5 ug estradiol cypionate in 0.1 ml sesame oil. One week after estrogen priming, the mice were treated with s.c. doses of palivizumab(NH, EAAAK)-relaxin(dual) (SEQ ID NO: 222) with palivizumab(LC) (SEQ ID NO: 7), purified in Example 22, (1.5, 3.0, 7.5, 15 nmol/kg). Interpubic ligament length was measured at 24 hours after dosing using a caliper. The fusion protein showed a dose-response efficacy down to 3.0 nmol/kg. FIG. 25 provides interpubic ligament lengths (mm) versus dosage of palivizumab(NH, EAAAK)-relaxin(dual) fusion protein.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All references cited herein are incorporated by reference in their entirety and for all purposes to the same extent as if each individual publication or patent or patent application was specifically and individually indicated to be incorporated by reference in its entirety for all purposes.

TABLE 1
Immunoglobulin Light Chain (LC) and Heavy Chain
(HC)Nucleotide Sequence

| NAME | SEQ ID NO | SEQUENCE |
|---|---|---|
| Trastuzumab L | 1 | GACATCCAGATGACCCAGTCTCCATCCTCCCTGTCTGCATCTGT |
| | | AGGAGACAGAGTCACCATCACTTGCCGGGCAAGTCAGGATGT |
| | | GAATACCGCGGTCGCATGGTATCAGCAGAAACCAGGGAAAGC |
| | | CCCTAAGCTCCTGATCTATTCTGCATCCTTCTTGTATAGTGGGG |
| | | TCCCATCAAGGTTCAGTGGCAGTAGATCTGGGACAGATTTCAC |
| | | TCTCACCATCAGCAGTCTGCAACCTGAAGATTTTGCAACTTAC |
| | | TACTGTCAACAGCATTACACTACCCCTCCGACGTTCGGCCAAG |
| | | GTACCAAGCTTGAGATCAAACGAACTGTGGCTGCACCATCTGT |
| | | CTTCATCTTCCCGCCATCTGATGAGCAGTTGAAATCTGGAACT |
| | | GCCTCTGTCGTGTGCCTGCTGAATAACTTCTATCCCAGAGAGG |
| | | CCAAAGTACAGTGGAAGGTGGATAACGCCCTCCAATCGGGTA |
| | | ACTCCCAGGAGAGTGTCACAGAGCAGGACAGCAAGGACAGCA |
| | | CCTACAGCCTCAGCAGCACCCTGACGCTGAGCAAAGCAGACT |
| | | ACGAGAAACACAAAGTCTACGCCTGCGAAGTCACCCATCAGG |
| | | GCCTGTCCTCGCCCGTCACAAAGAGCTTCAACAGGGGAGAGTG |
| | | T |
| Trastuzumab H | 2 | GAGGTGCAGCTGGTGGAGTCTGGAGGAGGCTTGGTCCAGCCT |
| | | GGGGGGTCCCTGAGACTCTCCTGTGCAGCCTCTGGGTTCAATA |
| | | TTAAGGACACTTACATCCACTGGGTCCGCCAGGCTCCAGGGAA |
| | | GGGGCTGGAGTGGGTCGCACGTATTTATCCTACCAATGGTTAC |
| | | ACACGCTACGCAGACTCCGTGAAGGGCCGATTCACCATCTCCG |
| | | CAGACACTTCCAAGAACACGGCGTATCTTCAAATGAACAGCCT |
| | | GAGAGCCGAGGACACGGCCGTGTATTACTGTTCGAGATGGGG |
| | | CGGTGACGGCTTCTATGCCATGGACTACTGGGGCCAAGGAACC |
| | | CTGGTCACCGTCTCCTCAGCCTCCACCAAGGGCCCATCGGTCT |
| | | TCCCCCTGGCACCCTCCTCCAAGAGCACCTCTGGGGGCACAGC |
| | | GGCCCTGGGCTGCCTGGTCAAGGACTACTTCCCCGAACCGGTG |
| | | ACGGTGTCGTGGAACTCAGGCGCCCTGACCAGCGGCGTGCAC |
| | | ACCTTCCCGGCTGTCCTACAGTCCTCAGGACTCTACTCCCTCAG |
| | | CAGCGTGGTGACTGTGCCCTCTAGCAGCTTGGGCACCCAGACC |
| | | TACATCTGCAACGTGAATCACAAGCCCAGCAACACCAAGGTG |
| | | GACAAGAAAGTTGAACCCAAATCTTGCGACAAAACTCACACA |
| | | TGCCCACCGTGCCCAGCACCTCCAGTCGCCGGACCGTCAGTCT |
| | | TCCTCTTCCCTCCAAAACCCAAGGACACCCTCATGATCTCCCG |
| | | GACCCCTGAGGTCACATGCGTGGTGGTGGACGTGAGCCACGA |
| | | AGACCCTGAGGTCAAGTTCAACTGGTACGTGGACGGCGTGGA |
| | | GGTGCATAATGCCAAGACAAAGCCGCGGGAGGAGCAGTACAA |
| | | CAGCACGTACCGTGTGGTCAGCGTCCTCACCGTCCTGCACCAG |
| | | GACTGGCTGAATGGCAAGGAGTACAAGTGCAAGGTCTCCAAC |
| | | AAAGGCCTCCCAAGCTCCATCGAGAAAACCATCTCCAAAGCC |
| | | AAAGGGCAGCCCCGAGAACCACAGGTGTACACCCTGCCTCCA |
| | | TCCCGGGATGAGCTGACCAAGAACCAGGTCAGCCTGACCTGCC |
| | | TGGTCAAAGGCTTCTATCCCAGCGACATCGCCGTGGAGTGGGA |
| | | GAGCAATGGGCAGCCGGAGAACAACTACAAGACCACGCCTCC |
| | | CGTGCTGGACTCCGACGGCTCCTTCTTCCTCTACAGCAAGCTC |
| | | ACCGTGGACAAGAGCAGGTGGCAGCAGGGGAACGTCTTCTCA |
| | | TGCTCCGTGATGCATGAGGCTCTGCACAACCACTACACGCAGA |
| | | AGAGCCTCTCCCTGTCTCCGGGTAAATGATAA |
| Palivizumab L | 3 | GACATCCAGATGACCCAGTCCCCCTCCACCCTGTCCGCCTCCG |
| | | TGGGCGACCGCGTGACCATCACCTGCAAGTGCCAGCTGTCCGT |
| | | GGGCTACATGCACTGGTACCAGCAGAAGCCCGGCAAGGCCCC |
| | | CAAGCTGCTGATCTACGACACCTCCAAGCTGGCCTCCGGCGTG |
| | | CCCTCCCGCTTCTCCGGCTCCGGCTCCGGCACCGAGTTCACCCT |
| | | GACCATCTCCTCCCTGCAGCCCGACGACTTCGCCACCTACTAC |
| | | TGCTTCCAGGGCTCCGGCTACCCCTTCACCTTCGGCGGCGGCA |
| | | CCAAGCTGGAGATCAAACGAACTGTGGCTGCACCATCTGTCTT |
| | | CATCTTCCCGCCATCTGATGAGCAGTTGAAATCTGGAACTGCC |
| | | TCTGTCGTGTGCCTGCTGAATAACTTCTATCCCAGAGAGGCCA |
| | | AAGTACAGTGGAAGGTGGATAACGCCCTCCAATCGGGTAACT |
| | | CCCAGGAGAGTGTCACAGAGCAGGACAGCAAGGACAGCACCT |
| | | ACAGCCTCAGCAGCACCCTGACGCTGAGCAAAGCAGACTACG |
| | | AGAAACACAAAGTCTACGCCTGCGAAGTCACCCATCAGGGCC |
| | | TGTCCTCGCCCGTCACAAAGAGCTTCAACAGGGGAGAGTGT |
| Palivizumab H | 4 | CAGGTGACCCTGCGCGAGTCCGGCCCTGCACTGGTGAAGCCCA |
| | | CCCAGACCCTGACCCTGACCTGCACCTTCTCCGGCTTCTCCCTG |
| | | TCCACCTCCGGCATGTCCGTGGGCTGGATCCGGCAGCCTCCCG |
| | | GCAAGGCCCTGGAGTGGCTGGCTGACATCTGGTGGGACGACA |
| | | AGAAGGACTACAACCCCTCCCTGAAGTCCCGCCTGACCATCTC |
| | | CAAGGACACCTCCAAGAACCAGGTGGTGCTGAAGGTGACCAA |
| | | CATGGACCCCGCCGACACCGCCACCTACTACTGCGCCCGCTCA |
| | | ATGATTACCAACTGGTACTTCGACGTGTGGGGAGCCGGTACCA |
| | | CCGTGACCGTGTCTTCCGCCTCCACCAAGGGCCCATCGGTCTT |
| | | CCCCCTGGCACCCTCCTCCAAGAGCACCTCTGGGGGCACAGCG |
| | | GCCCTGGGCTGCCTGGTCAAGGACTACTTCCCCGAACCGGTGA |
| | | CGGTGTCGTGGAACTCAGGCGCCCTGACCAGCGGCGTGCACAC |
| | | CTTCCCGGCTGTCCTACAGTCCTCAGGACTCTACTCCCTCAGCA |
| | | GCGTGGTGACTGTGCCCTCTAGCAGCTTGGGCACCCAGACCTA |
| | | CATCTGCAACGTGAATCACAAGCCCAGCAACACCAAGGTGGA |
| | | CAAGAAAGTTGAACCCAAATCTTGCGACAAAACTCACACATG |
| | | CCCACCGTGCCCAGCACCTCCAGTCGCCGGACCGTCAGTCTTC |
| | | CTCTTCCCTCCAAAACCCAAGGACACCCTCATGATCTCCCGGA |
| | | CCCCTGAGGTCACATGCGTGGTGGTGGACGTGAGCCACGAAG |
| | | ACCCTGAGGTCAAGTTCAACTGGTACGTGGACGGCGTGGAGGT |
| | | GCATAATGCCAAGACAAAGCCGCGGGAGGAGCAGTACAACAG |
| | | CACGTACCGTGTGGTCAGCGTCCTCACCGTCCTGCACCAGGAC |
| | | TGGCTGAATGGCAAGGAGTACAAGTGCAAGGTCTCCAACAAA |
| | | GGCCTCCCAAGCTCCATCGAGAAAACCATCTCCAAAGCCAAA |
| | | GGGCAGCCCCGAGAACCACAGGTGTACACCCTGCCTCCATCCC |
| | | GGGATGAGCTGACCAAGAACCAGGTCAGCCTGACCTGCCTGG |
| | | TCAAAGGCTTCTATCCCAGCGACATCGCCGTGGAGTGGGAGAG |
| | | CAATGGGCAGCCGGAGAACAACTACAAGACCACGCCTCCCGT |
| | | GCTGGACTCCGACGGCTCCTTCTTCCTCTACAGCAAGCTCACC |
| | | GTGGACAAGAGCAGGTGGCAGCAGGGGAACGTCTTCTCATGC |
| | | TCCGTGATGCATGAGGCTCTGCACAACCACTACACGCAGAAGA |
| | | GCCTCTCCCTGTCTCCGGGTAAATGATAA |

TABLE 2
Immunoglobulin Light Chain (LC) and Heavy
Chain (HC)-Amino Acid Sequence

| Name | SEQ ID NO | Sequence |
|---|---|---|
| Trastuzumab L | 5 | DIQMTQSPSSLSASVGDRVTITCRASQDVNTAVAWYQQKPGKAP |
| | | KLLIYSASFLYSGVPSRFSGSRSGTDFTLTISSLQPEDFATYYCQQH |
| | | YTTPPTFGQGTKLEIKRTVAAPSVFIFPPSDEQLKSGTASVVCLLN |
| | | NFYPREAKVQWKVDNALQSGNSQESVTEQDSKDSTYSLSSTLTL |
| | | SKADYEKHKVYACEVTHQGLSSPVTKSFNRGEC |
| Trastuzumab H | 6 | EVQLVESGGGLVQPGGSLRLSCAASGFNIKDTYIHWVRQAPGKG |
| | | LEWVARIYPTNGYTRYADSVKGRFTISADTSKNTAYLQMNSLRA |
| | | EDTAVYYCSRWGGDGFYAMDYWGQGTLVTVSSASTKGPSVFPL |
| | | APSSKSTSGGTAALGCLVKDYFPEPVTVSWNSGALTSGVHTFPAV |
| | | LQSSGLYSLSSVVTVPSSSLGTQTYICNVNHKPSNTKVDKKVEPK |
| | | SCDKTHTCPPCPAPPVAGPSVFLFPPKPKDTLMISRTPEVTCVVVD |
| | | VSHEDPEVKFNWYVDGVEVHNAKTKPREEQYNSTYRVVSVLTV |
| | | LHQDWLNGKEYKCKVSNKGLPSSIEKTISKAKGQPREPQVYTLPP |
| | | SRDELTKNQVSLTCLVKGFYPSDIAVEWESNGQPENNYKTTPPVL |
| | | DSDGSFFLYSKLTVDKSRWQQGNVFSCSVMHEALHNHYTQKSLS |
| | | LSPGK |
| Palivizumab L | 7 | DIQMTQSPSTLSASVGDRVTITCKCQLSVGYMHWYQQKPGKAPK |
| | | LLIYDTSKLASGVPSRFSGSGSGTEFTLTISSLQPDDFATYYCFQGS |
| | | GYPFTFGGGTKLEIKRTVAAPSVFIFPPSDEQLKSGTASVVCLLNN |
| | | FYPREAKVQWKVDNALQSGNSQESVTEQDSKDSTYSLSSTLTLS |
| | | KADYEKHKVYACEVTHQGLSSPVTKSFNRGEC |
| Palivizumab H | 8 | QVTLRESGPALVKPTQTLTLTCTFSGFSLSTSGMSVGWIRQPPGK |
| | | ALEWLADIWWDDKKDYNPSLKSRLTISKDTSKNQVVLKVTNMD |
| | | PADTATYYCARSMITNWYFDVWGAGTTVTVSSASTKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKS |
| | | CDKTHTCPPCPAPPVAGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKGLPSSIEKTISKAKGQPREPQVYTLPPS |
| | | RDELTKNQVSLTCLVKGFYPSDIAVEWESNGQPENNYKTTPPVLD |
| | | SDGSFFLYSKLTVDKSRWQQGNVFSCSVMHEALHNHYTQKSLSL |
| | | SPGK |

TABLE 3
Immunoglobulin fusion protein - Nucleotide Sequence

| NAME | SEQ ID NO | SEQUENCE |
|---|---|---|
| Trastuzumab(NL, GGGGS) | 9 | CACGGAGAAGGAACATTTACCAGCGACCTCAGCAAGCAGATGGAG |
| Exendin-4 | | GAAGAGGCCGTGAGGCTGTTCATCGAGTGGCTGAAGAACGGCGG |
| | | 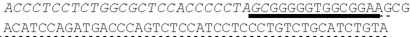 |
| | | 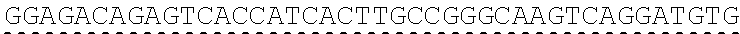 |
| | | 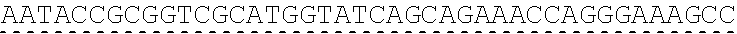 |
| | | 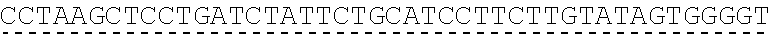 |
| | | 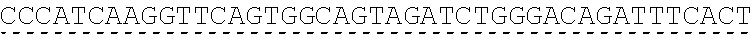 |
| | | 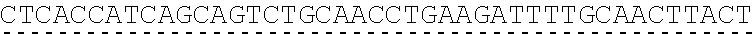 |
| | | 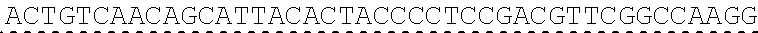 |
| | | 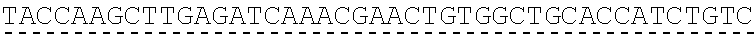 |
| | | 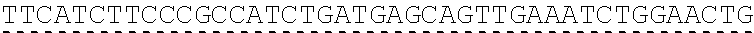 |
| | | 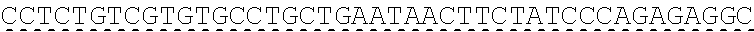 |
| | | 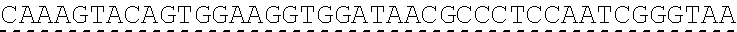 |
| | | 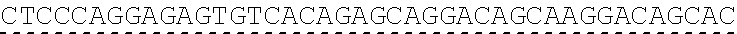 |
| | | 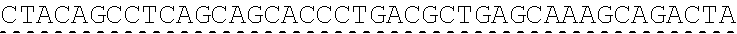 |
| | | 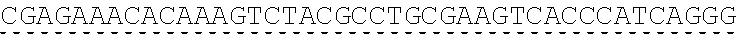 |
| | | 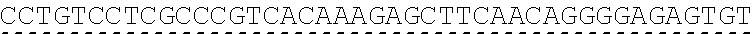 |
| Trastuzumab(CDR2H) Leptin | 10 | 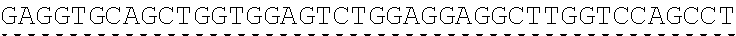 |
| | | 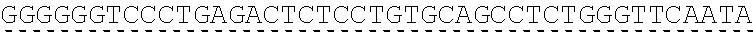 |
| | | 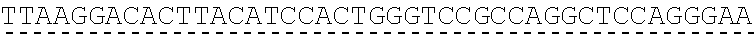 |
| | | 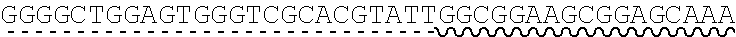 |
| | | 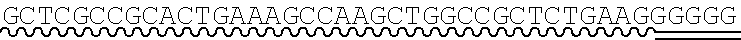 |
| | | TGGCGGAAGCGTTCCAATTCAAAAGGTTCAAGATGATACCAAAACT |
| | | CTGATTAAAACTATTGTCACGCGTATAAACGACATCTCACATACCCA |
| | | GTCGGTTAGCTCAAAGCAAAAAGTTACCGGTTTGGACTTTATTCCG |
| | | GGACTGCACCCGATCCTGACCCTTAGTAAAATGGACCAGACACTG |
| | | GCCGTCTACCAGCAAATCCTGACATCGATGCCATCCAGAAATGTGA |
| | | TACAAATTAGCAACGATTTGGAAAACCTTCGCGATCTGCTGCACGT |
| | | GCTGGCCTTCAGTAAGTCCTGTCATCTGCCGTGGGCGTCGGGACT |
| | | GGAGACTCTTGACTCGCTGGGTGGAGTGTTAGAGGCCTCTGGCTA |
| | | TTCTACTGAAGTCGTTGCGCTGTCACGCCTCCAGGGGAGCCTGCA |
| | | GGACATGCTGTGGCAGCTGGACCTGTCACCTGGCTGCGGCGGAG |
| | | 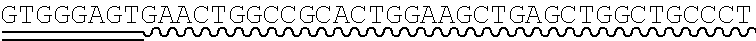 |
| | | 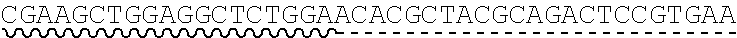 |
| | | 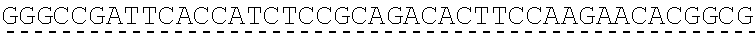 |
| | | 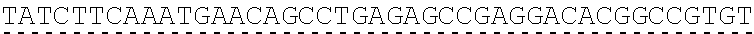 |
| | | 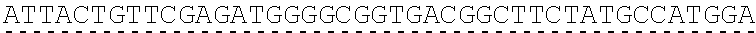 |
| | | 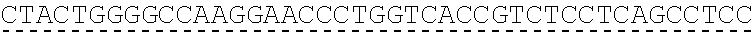 |
| | | 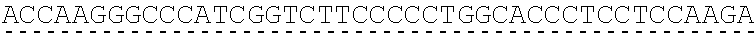 |
| | | 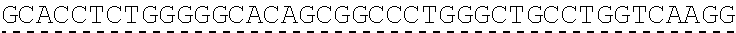 |
| | | 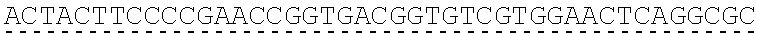 |
| | | 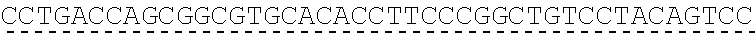 |
| | | 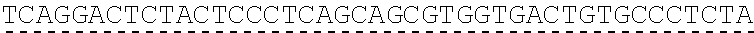 |
| | | 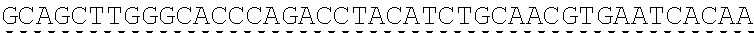 |
| | | 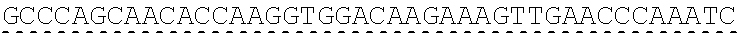 |
| | | 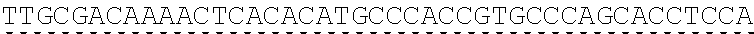 |
| | | 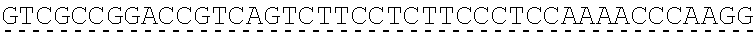 |
| | | 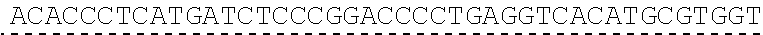 |
| | | 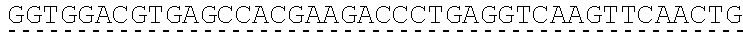 |
| | | 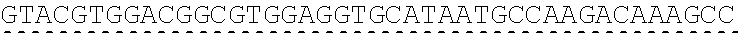 |
| | | 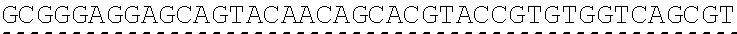 |
| | | 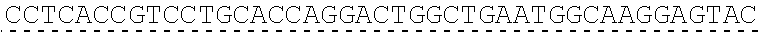 |
| | | 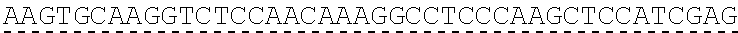 |
| | | 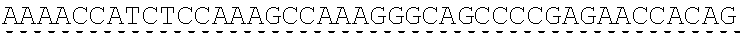 |
| | | 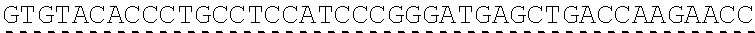 |
| | | 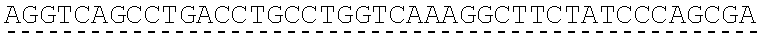 |
| | | 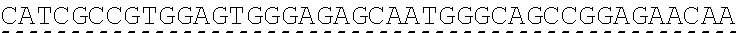 |
| | | 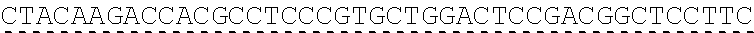 |
| | | 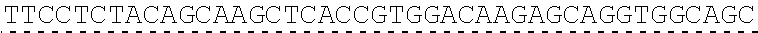 |
| | | 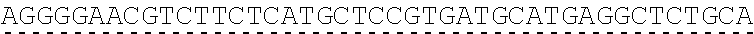 |
| | | 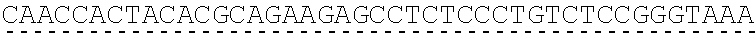 |
| | | 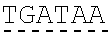 |
| Trastuzumab (CDR3H) Leptin | 11 | 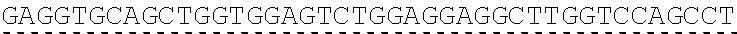 |
| | | 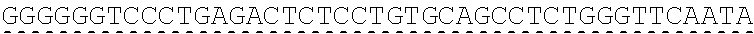 |
| | | 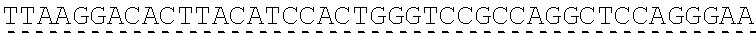 |
| | | 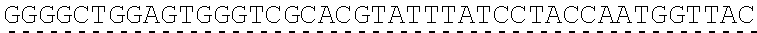 |
| | | 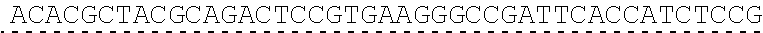 |
| | | 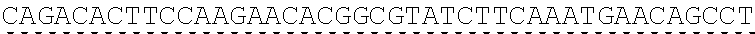 |
| | | 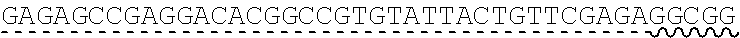 |
| | | 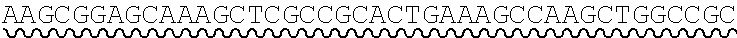 |
| | | 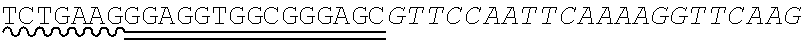 |
| | | ATGATACCAAAACTCTGATTAAAACTATTGTCACGCGTATAAACGAC |
| | | ATCTCACATACCCAGTCGGTTAGCTCAAAGCAAAAAGTTACCGGTT |
| | | TGGACTTTATTCCGGGACTGCACCCGATCCTGACCCTTAGTAAAAT |
| | | GGACCAGACACTGGCCGTCTACCAGCAAATCCTGACATCGATGCC |
| | | ATCCAGAAATGTGATACAAATTAGCAACGATTTGGAAAACCTTCGC |
| | | GATCTGCTGCACGTGCTGGCCTTCAGTAAGTCCTGTCATCTGCCGT |
| | | GGGCGTCGGGACTGGAGACTCTTGACTCGCTGGGTGGAGTGTTAG |
| | | AGGCCTCTGGCTATTCTACTGAAGTCGTTGCGCTGTCACGCCTCCA |
| | | GGGGAGCCTGCAGGACATGCTGTGGCAGCTGGACCTGTCACCTG |
| | | 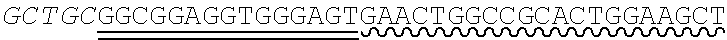 |
| | | 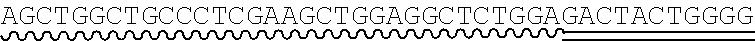 |
| | | 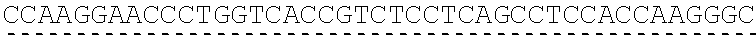 |
| | | 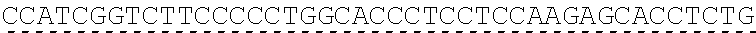 |
| | | 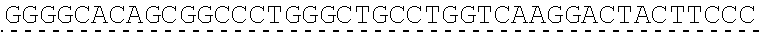 |
| | | 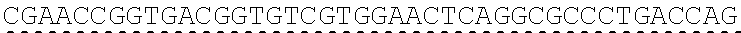 |
| | | 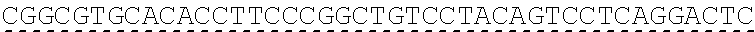 |
| | | 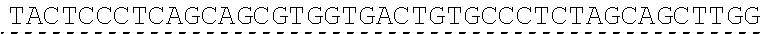 |
| | | 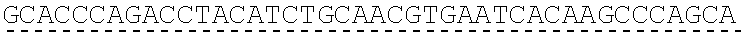 |
| | | 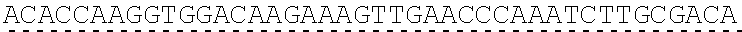 |
| | | 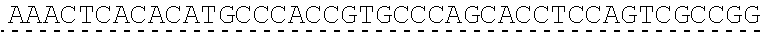 |
| | | 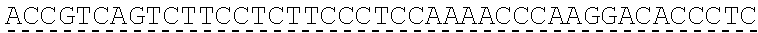 |
| | | 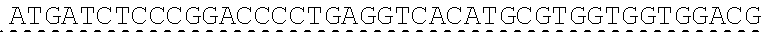 |
| | | 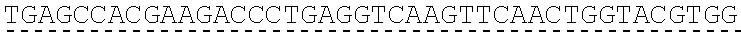 |
| | | 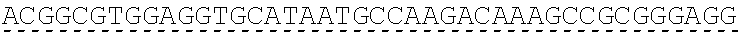 |
| | | 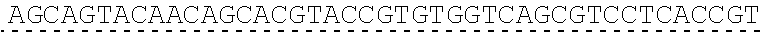 |
| | | 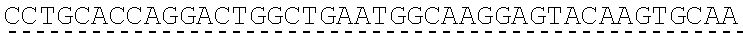 |
| | | 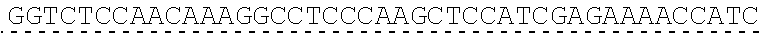 |
| | | 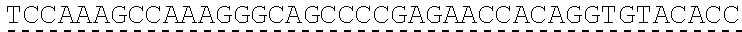 |
| | | 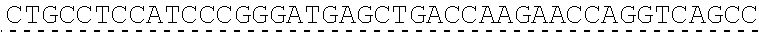 |
| | | 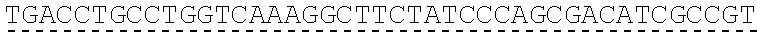 |
| | | 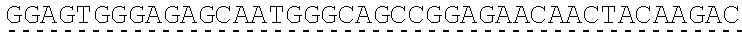 |
| | | 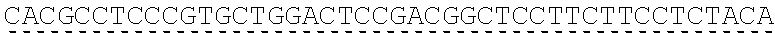 |
| | | 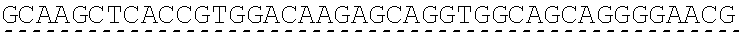 |
| | | 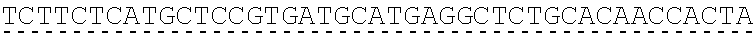 |
| | | 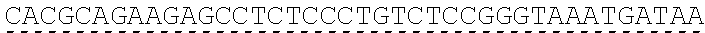 |
| Trastuzumab (NL, GGGGS)ZP1 | 12 | CACAGCCAGGGCACATTCACTAGCGATTATAGTAAATATCTGGATT |
| | | 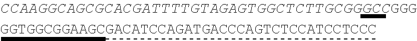 |
| | | 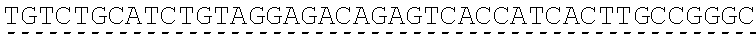 |
| | | 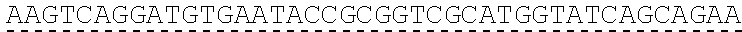 |
| | | 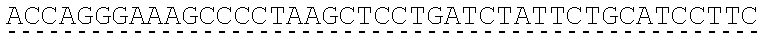 |
| | | 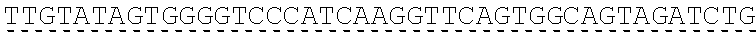 |
| | | 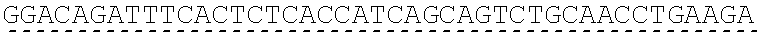 |
| | | 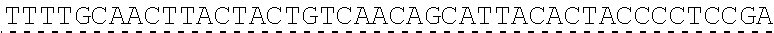 |
| | | 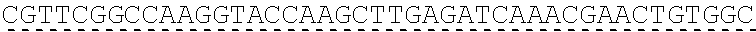 |
| | | 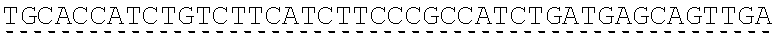 |
| | | 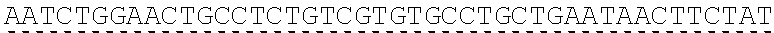 |
| | | 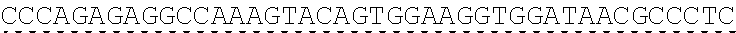 |
| | | 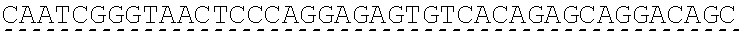 |
| | | 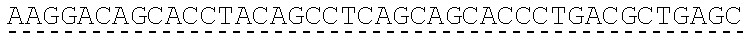 |
| | | 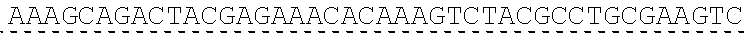 |
| | | 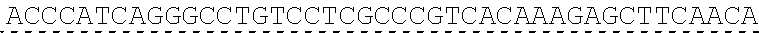 |
| | | 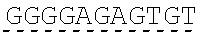 |
| Trastuzumab (NL, GGGGS) ZPCEX | 13 | CACAGCCAGGGCACATTCACTAGCGATTATAGTAAATATCTGGATT |
| | | CCAAGGCAGCGCACGATTTTGTAGAGTGGCTCTTGAACGGAGGCC |
| | | 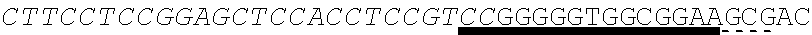 |
| | | 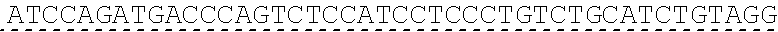 |
| | | 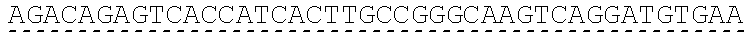 |
| | | 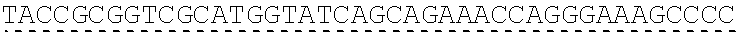 |
| | | 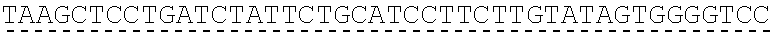 |
| | | 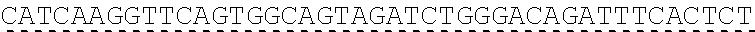 |
| | | 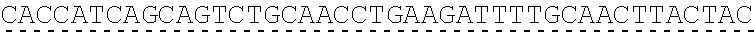 |
| | | 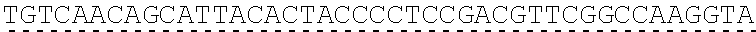 |
| | | 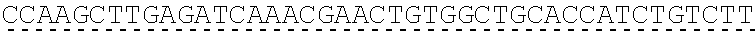 |
| | | 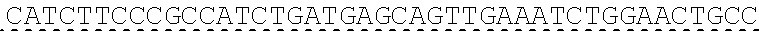 |
| | | 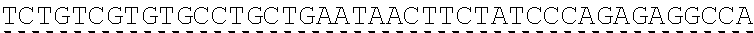 |
| | | 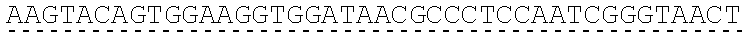 |
| | | 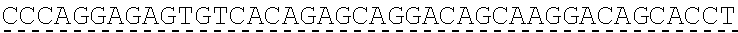 |
| | | 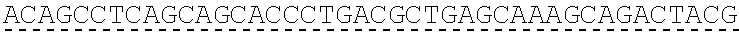 |
| | | 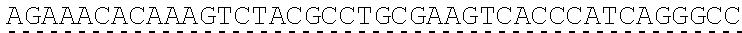 |
| | | 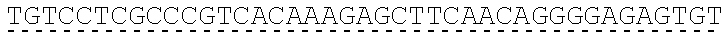 |
| Trastuzumab (NL, GGGGG) ZPCEX | 14 | CACAGCCAGGGCACATTCACTAGCGATTATAGTAAATATCTGGATT |
| | | CCAAGGCAGCGCACGATTTTGTAGAGTGGCTCTTGAACGGAGGCC |
| | | 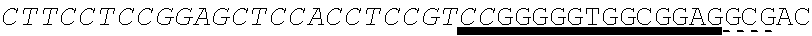 |
| | | 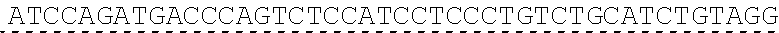 |
| | | 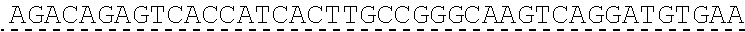 |
| | | 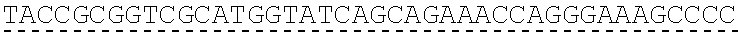 |
| | | 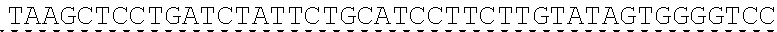 |
| | | 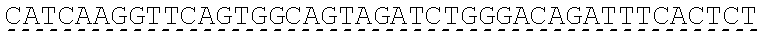 |
| | | 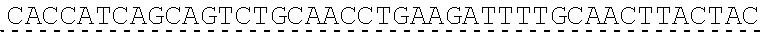 |
| | | 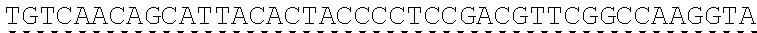 |
| | | 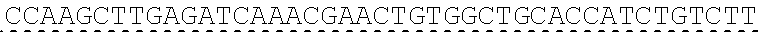 |
| | | 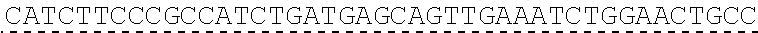 |
| | | 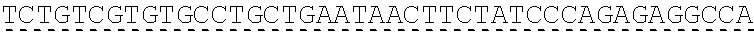 |
| | | 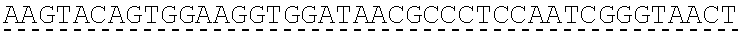 |
| | | 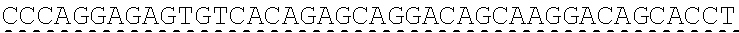 |
| | | 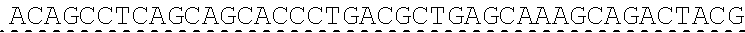 |
| | | 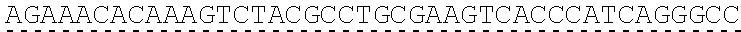 |
| | | 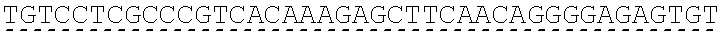 |
| Palivizumab (NL, GGGGS) ZPCEX | 15 | CACAGCCAGGGCACATTCACTAGCGATTATAGTAAATATCTGGATT |
| | | CCAAGGCAGCGCACGATTTTGTAGAGTGGCTCTTGAACGGAGGCC |
| | | 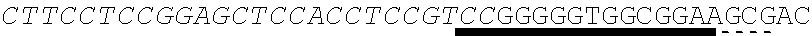 |
| | | 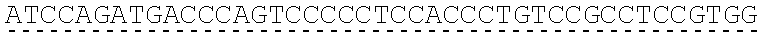 |
| | | 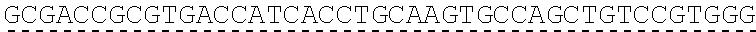 |
| | | 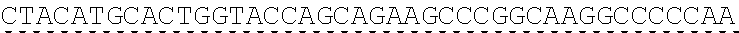 |
| | | 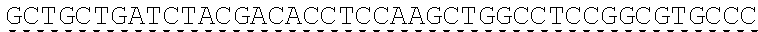 |
| | | 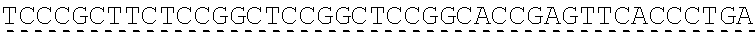 |
| | | 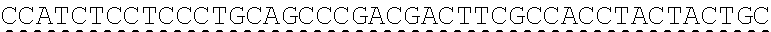 |
| | | 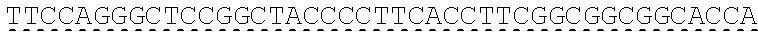 |
| | | 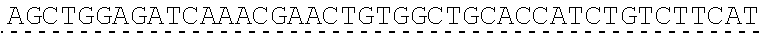 |
| | | 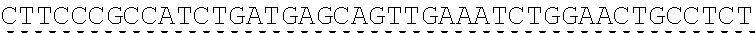 |
| | | 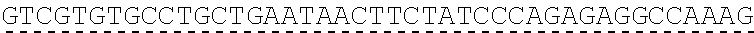 |
| | | 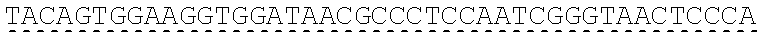 |
| | | 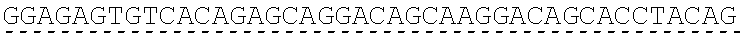 |
| | | 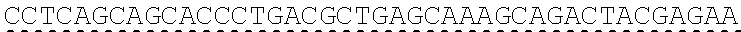 |
| | | 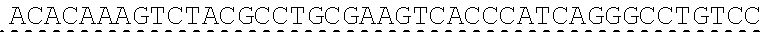 |
| | | 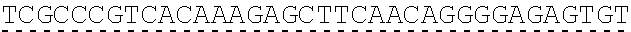 |
| Palivizumab (NL, GGGGG) ZPCEX | 16 | CACAGCCAGGGCACATTCACTAGCGATTATAGTAAATATCTGGATT |
| | | CCAAGGCAGCGCACGATTTTGTAGAGTGGCTCTTGAACGGAGGCC |
| | | 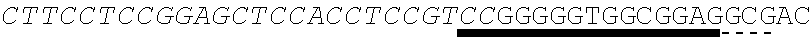 |
| | | 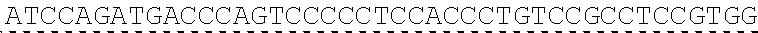 |
| | | 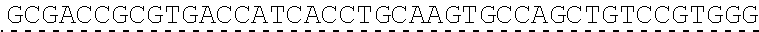 |
| | | 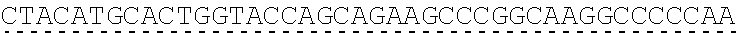 |
| | | 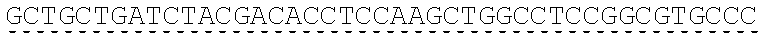 |
| | | 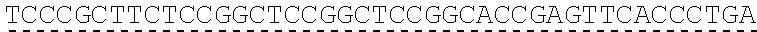 |
| | | 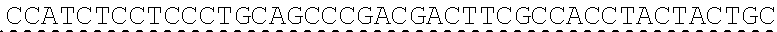 |
| | | 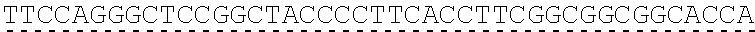 |
| | | 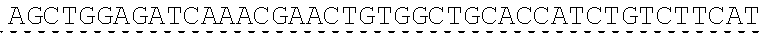 |
| | | 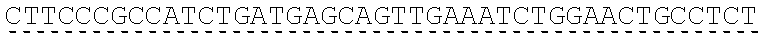 |
| | | 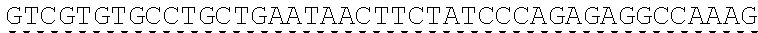 |
| | | 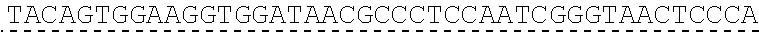 |
| | | 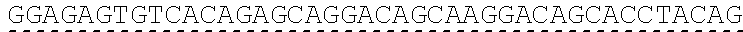 |
| | | 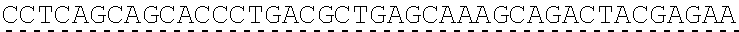 |
| | | 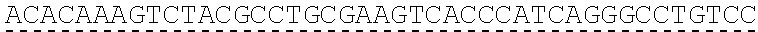 |
| | | 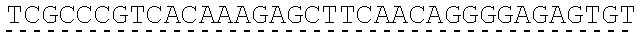 |
| Palivizumab (NH, GGGGS) ZPCEX | 17 | CACAGCCAGGGCACATTCACTAGCGATTATAGTAAATATCTGGATT |
| | | CCAAGGCAGCGCACGATTTTGTAGAGTGGCTCTTGAACGGAGGCC |
| | | 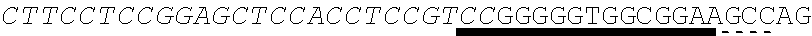 |
| | | 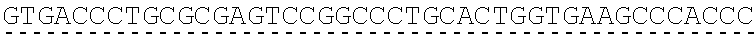 |
| | | 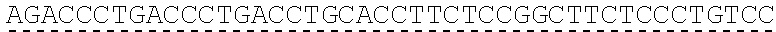 |
| | | 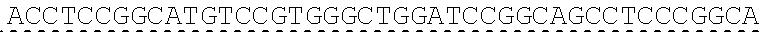 |
| | | 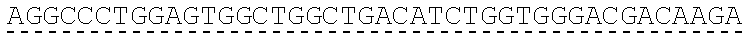 |
| | | 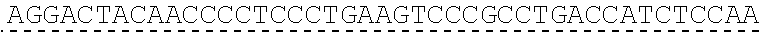 |
| | | 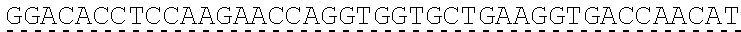 |
| | | 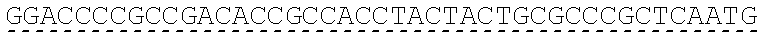 |
| | | 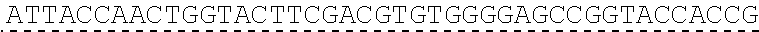 |
| | | 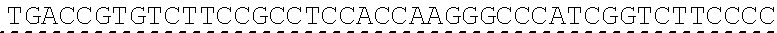 |
| | | 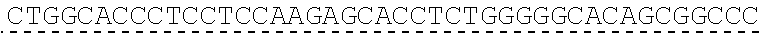 |
| | | 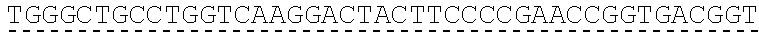 |
| | | 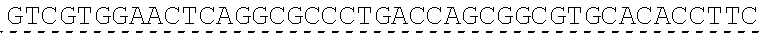 |
| | | 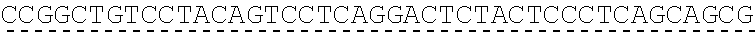 |
| | | 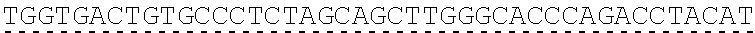 |
| | | 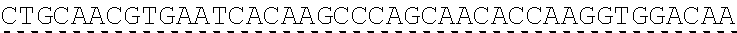 |
| | | 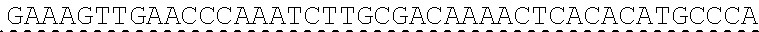 |
| | | 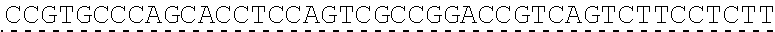 |
| | | 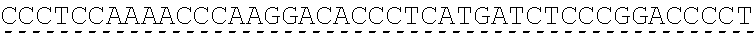 |
| | | 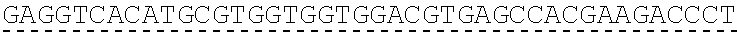 |
| | | 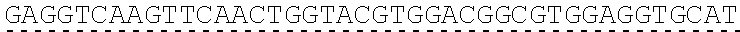 |
| | | 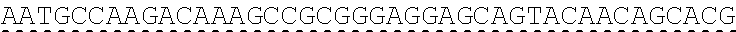 |
| | | 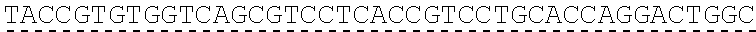 |
| | | 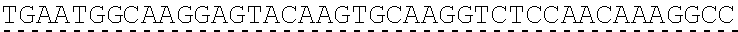 |
| | | 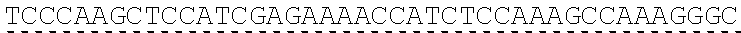 |
| | | 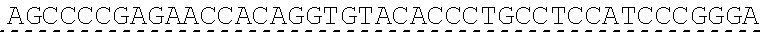 |
| | | 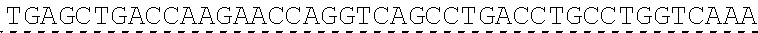 |
| | | 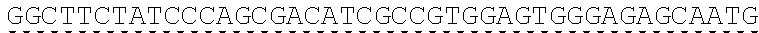 |
| | | 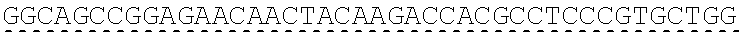 |
| | | 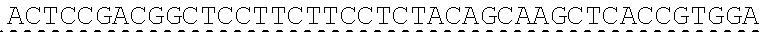 |
| | | 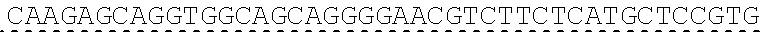 |
| | | 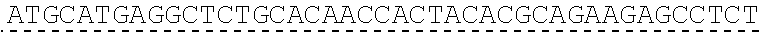 |
| | | 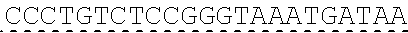 |
| Trastuzumab (NL, GGGGS) | 18 | GACTCTTGGATGGAAGAAGTTATCAAACTGTGCGGTCGTGAACTGG |
| Relaxin2 | | TTCGTGCTCAGATCGCTATCTGCGGTATGTCTACCTGGTCTAAACG |
| | | TTCTCTGTCTCAGGAAGACGCTCCGCAGACCCCGCGTCCGGTTGC |
| | | TGAAATCGTTCCGTCTTTCATCAACAAAGACACCGAAACCATCAACA |
| | | TGATGTCTGAATTCGTTGCTAACCTGCCGCAGGAACTGAAACTGAC |
| | | CCTGTCTGAAATGCAGCCGGCTCTGCCGCAGCTGCAGCAGCACGT |
| | | TCCGGTTCTGAAAGACTCTTCTCTGCTGTTCGAAGAATTCAAAAAAC |
| | | TGATCCGTAACCGTCAGTCTGAAGCTGCTGACTCTTCTCCGTCTGA |
| | | ACTGAAATACCTGGGTCTGGACACCCACTCTCGTAAAAAACGTCAG |
| | | CTGTACTCTGCTCTGGCTAACAAATGCTGCCACGTTGGTTGCACCA |
| | | 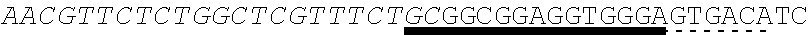 |
| | | 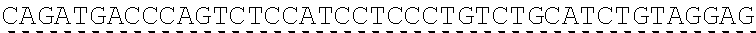 |
| | | 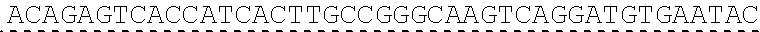 |
| | | 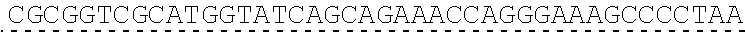 |
| | | 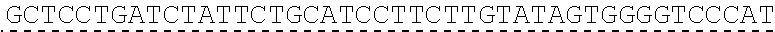 |
| | | 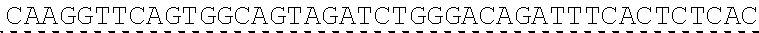 |
| | | 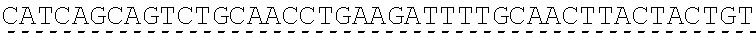 |
| | | 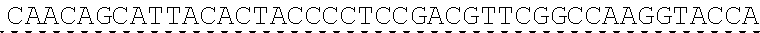 |
| | | 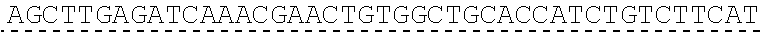 |
| | | 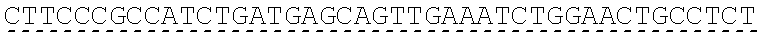 |
| | | 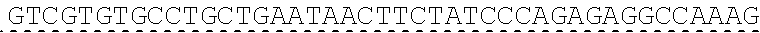 |
| | | 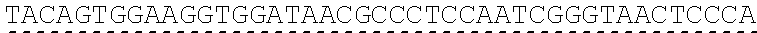 |
| | | 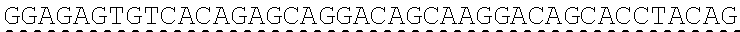 |
| | | 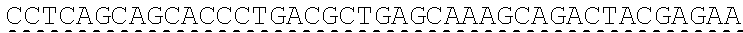 |
| | | 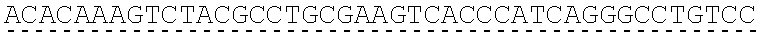 |
| | | 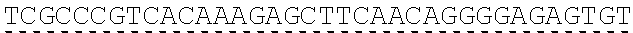 |
| Trastuzumab (NL, GGGGS) | 19 | GACTCTTGGATGGAAGAAGTTATCAAACTGTGCGGTCGTGAACTGG |
| Relaxin2 (XT100) | | TTCGTGCTCAGATCGCTATCTGCGGTATGTCTACCTGGTCTAAACG |
| | | 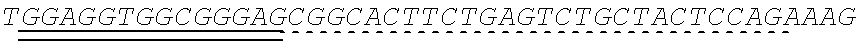 |
| | | 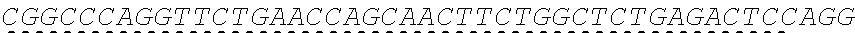 |
| | | 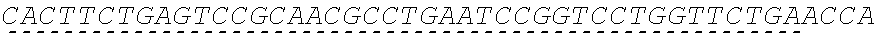 |
| | | 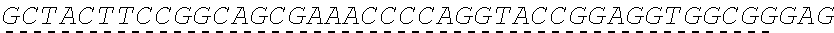 |
| | | 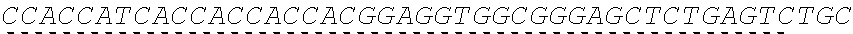 |
| | | 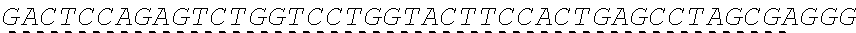 |
| | | 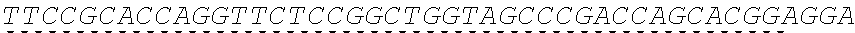 |
| | | 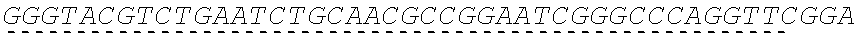 |
| | | 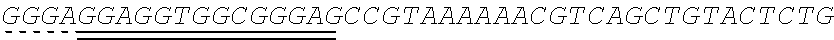 |
| | | CTCTGGCTAACAAATGCTGCCACGTTGGTTGCACCAAACGTTCTCT |
| | | 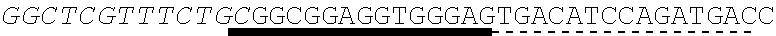 |
| | | 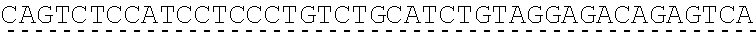 |
| | | 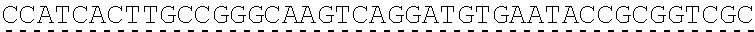 |
| | | 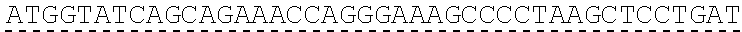 |
| | | 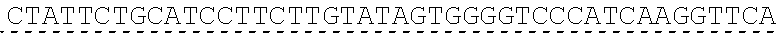 |
| | | 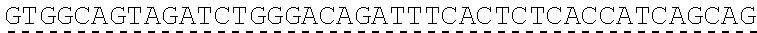 |
| | | 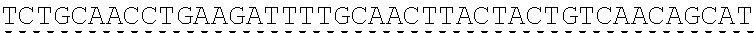 |
| | | 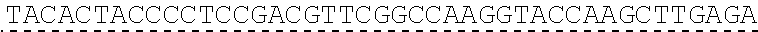 |
| | | 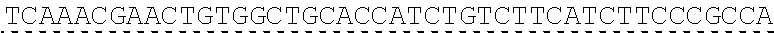 |
| | | 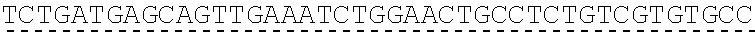 |
| | | 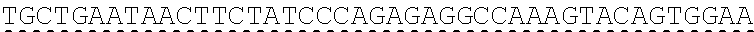 |
| | | 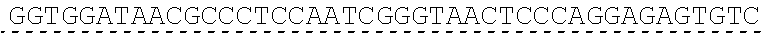 |
| | | 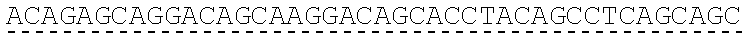 |
| | | 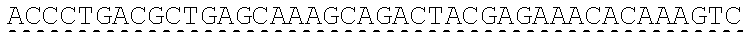 |
| | | 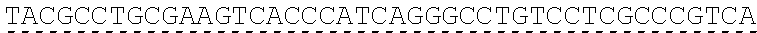 |
| | | 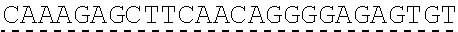 |
| Trastuzumab (NL, GGGGS) | 20 | GACTCTTGGATGGAAGAAGTTATCAAACTGTGCGGTCGTGAACTGG |
| Relaxin2 (XT35) | | TTCGTGCTCAGATCGCTATCTGCGGTATGTCTACCTGGTCTAAACG |
| | | 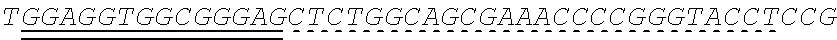 |
| | | 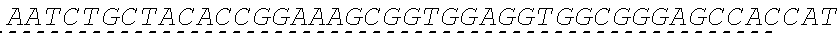 |
| | | 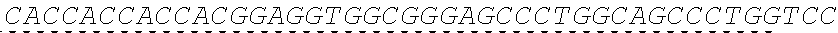 |
| | | 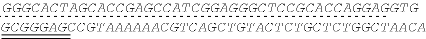 |
| | | AATGCTGCCACGTTGGTTGCACCAAACGTTCTCTGGCTCGTTTCTG |
| | | 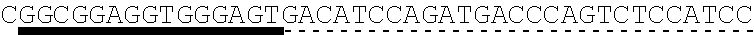 |
| | | 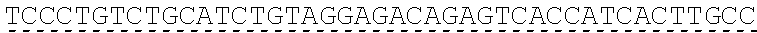 |
| | | 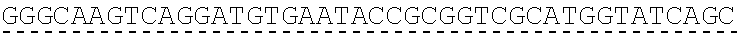 |
| | | 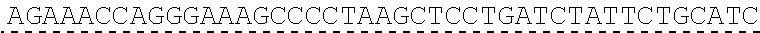 |
| | | 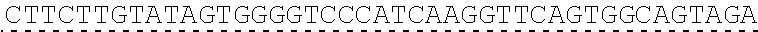 |
| | | 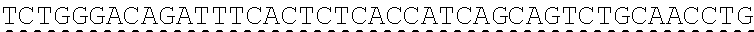 |
| | | 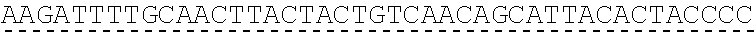 |
| | | 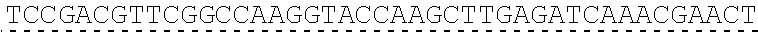 |
| | | 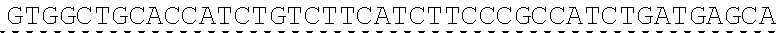 |
| | | 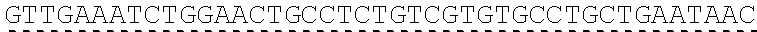 |
| | | 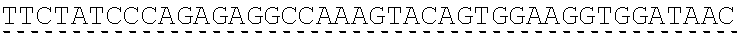 |
| | | 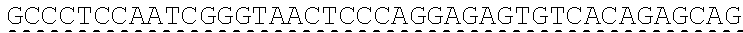 |
| | | 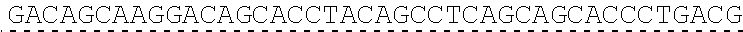 |
| | | 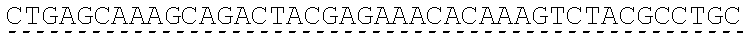 |
| | | 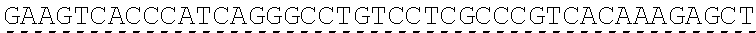 |
| | | 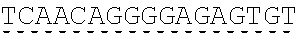 |
| Trastuzumab (NL, GGGGG) | 21 | GACTCTTGGATGGAAGAAGTTATCAAACTGTGCGGTCGTGAACTGG |
| Relaxin2 (XT35) | | TTCGTGCTCAGATCGCTATCTGCGGTATGTCTACCTGGTCTAAACG |
| | | 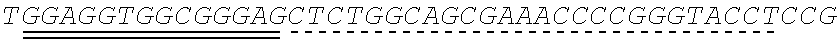 |
| | | 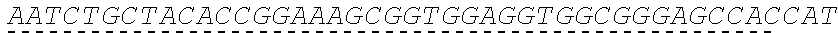 |
| | | 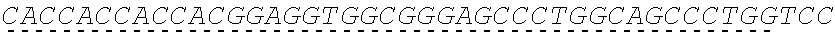 |
| | | 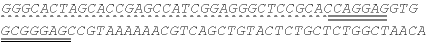 |
| | | AATGCTGCCACGTTGGTTGCACCAAACGTTCTCTGGCTCGTTTCTG |
| | | 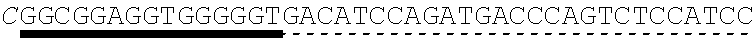 |
| | | 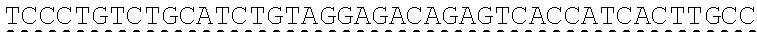 |
| | | 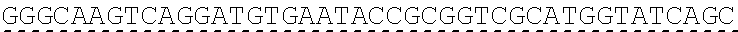 |
| | | 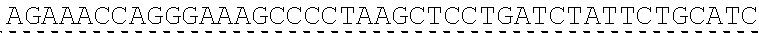 |
| | | 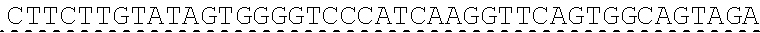 |
| | | 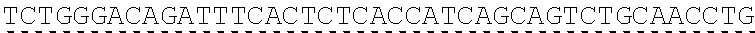 |
| | | 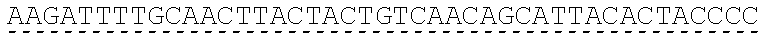 |
| | | 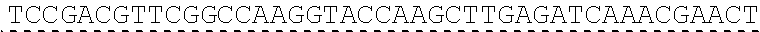 |
| | | 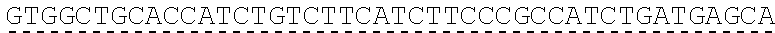 |
| | | 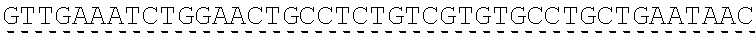 |
| | | 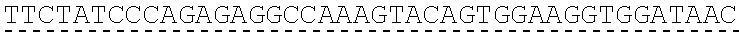 |
| | | 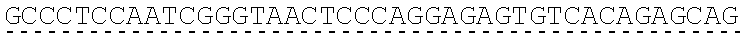 |
| | | 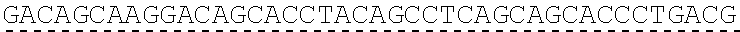 |
| | | 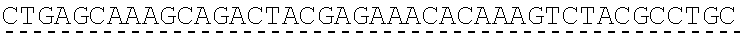 |
| | | 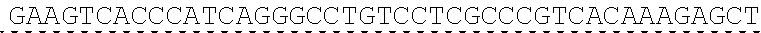 |
| | | 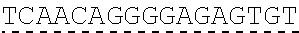 |
| Trastuzumab (NL, CEXGGGGG) | 22 | GACTCTTGGATGGAAGAAGTTATCAAACTGTGCGGTCGTGAACTGG |
| Relaxin2 (XT35) | | TTCGTGCTCAGATCGCTATCTGCGGTATGTCTACCTGGTCTAAACG |
| | | 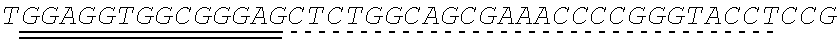 |
| | | 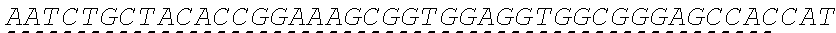 |
| | | 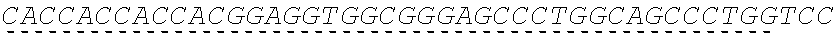 |
| | | 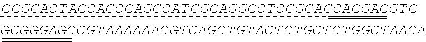 |
| | | AATGCTGCCACGTTGGTTGCACCAAACGTTCTCTGGCTCGTTTCTG |
| | | 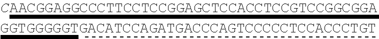 |
| | | 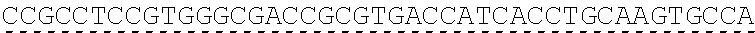 |
| | | 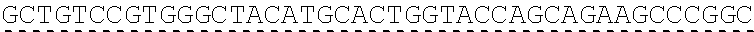 |
| | | 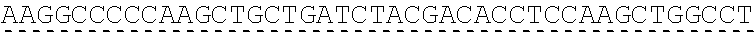 |
| | | 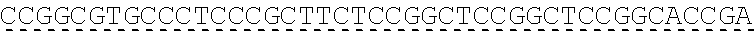 |
| | | 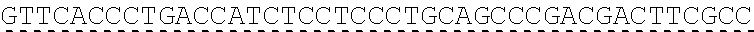 |
| | | 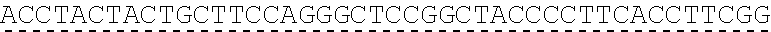 |
| | | 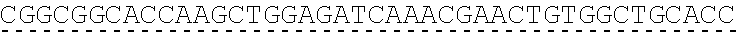 |
| | | 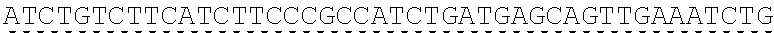 |
| | | 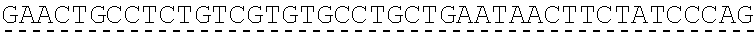 |
| | | 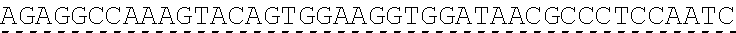 |
| | | 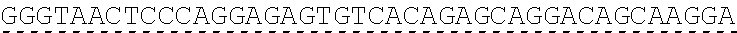 |
| | | 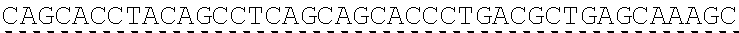 |
| | | 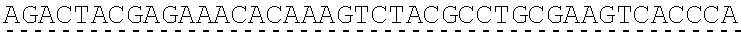 |
| | | 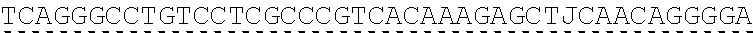 |
| | | 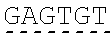 |
| Palivizumab (NL, GGGGS) | 23 | GACTCTTGGATGGAAGAAGTTATCAAACTGTGCGGTCGTGAACTGG |
| Relaxin2 (XT35) | | TTCGTGCTCAGATCGCTATCTGCGGTATGTCTACCTGGTCTAAACG |
| | | 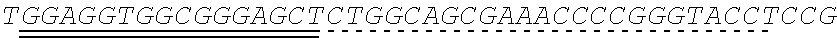 |
| | | 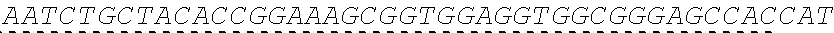 |
| | | 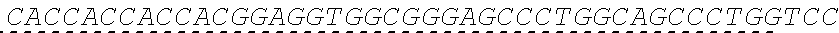 |
| | | 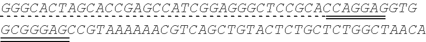 |
| | | AATGCTGCCACGTTGGTTGCACCAAACGTTCTCTGGCTCGTTTCTG |
| | | 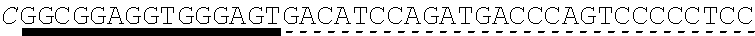 |
| | | 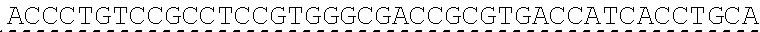 |
| | | 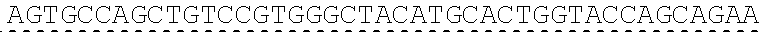 |
| | | 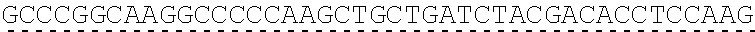 |
| | | 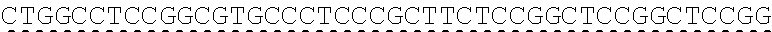 |
| | | 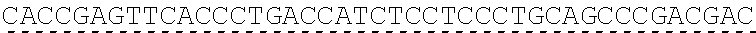 |
| | | 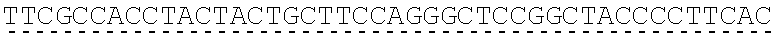 |
| | | 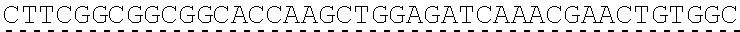 |
| | | 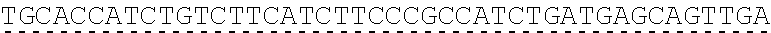 |
| | | 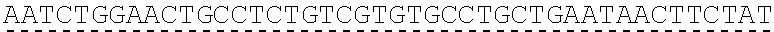 |
| | | 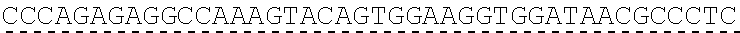 |
| | | 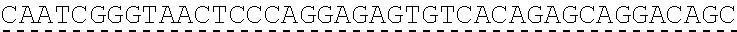 |
| | | 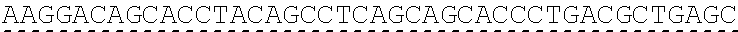 |
| | | 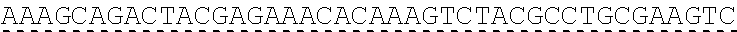 |
| | | 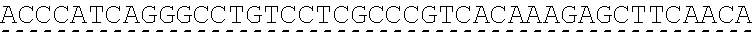 |
| | | 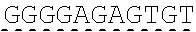 |
| Palivizumab (NL, GGGGG) | 24 | GACTCTTGGATGGAAGAAGTTATCAAACTGTGCGGTCGTGAACTGG |
| Relaxin2 (XT35) | | TTCGTGCTCAGATCGCTATCTGCGGTATGTCTACCTGGTCTAAACG |
| | | 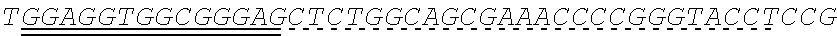 |
| | | 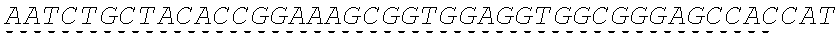 |
| | | 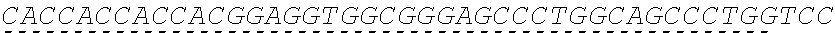 |
| | | 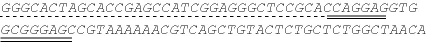 |
| | | AATGCTGCCACGTTGGTTGCACCAAACGTTCTCTGGCTCGTTTCTG |
| | | 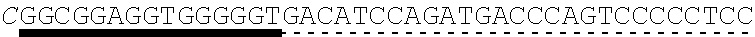 |
| | | 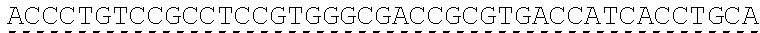 |
| | | 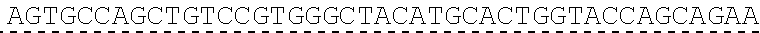 |
| | | 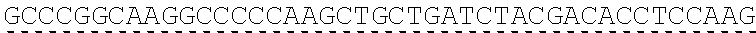 |
| | | 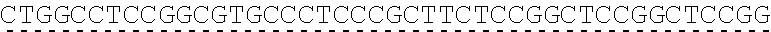 |
| | | 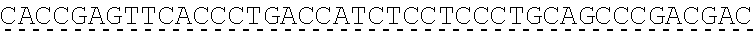 |
| | | 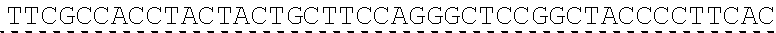 |
| | | 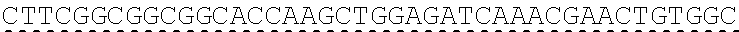 |
| | | 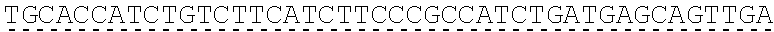 |
| | | 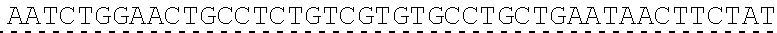 |
| | | 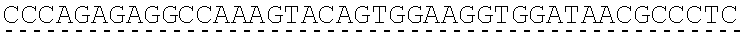 |
| | | 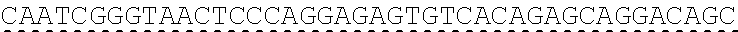 |
| | | 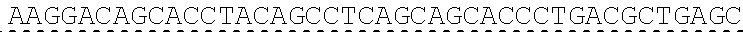 |
| | | 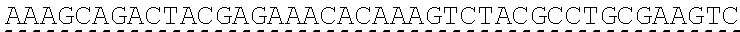 |
| | | 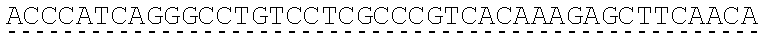 |
| | | 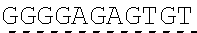 |
| Palivizumab (NL, | 25 | GACTCTTGGATGGAAGAAGTTATCAAACTGTGCGGTCGTGAACTGG |
| CEXGGGGG) Relaxin2 (XT35) | | TTCGTGCTCAGATCGCTATCTGCGGTATGTCTACCTGGTCTAAACG |
| | | 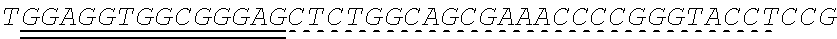 |
| | | 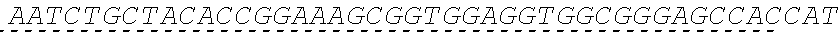 |
| | | 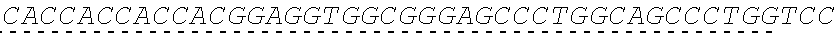 |
| | | 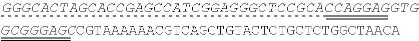 |
| | | AATGCTGCCACGTTGGTTGCACCAAACGTTCTCTGGCTCGTTTCTG |
| | | 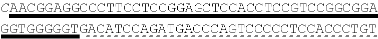 |
| | | 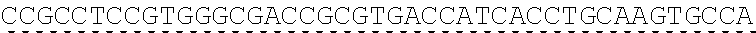 |
| | | 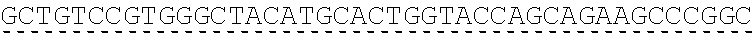 |
| | | 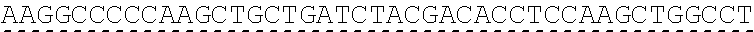 |
| | | 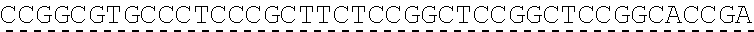 |
| | | 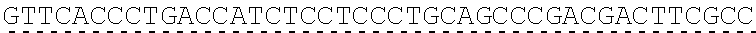 |
| | | 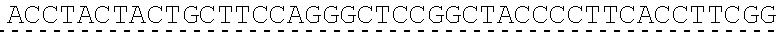 |
| | | 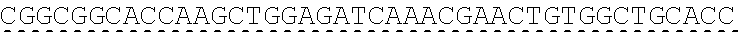 |
| | | 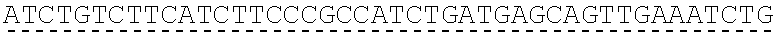 |
| | | 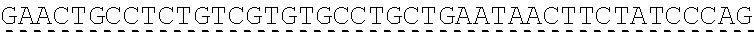 |
| | | 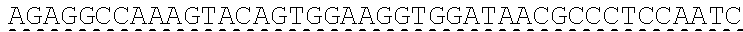 |
| | | 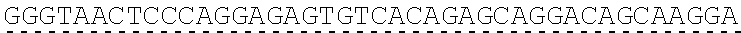 |
| | | 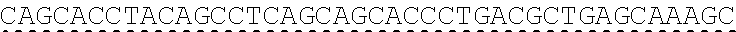 |
| | | 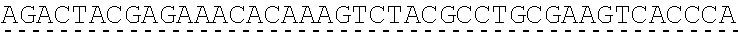 |
| | | 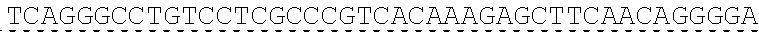 |
| | | 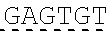 |
| Trastuzumab (NL, GGGGS) | 26 | GACTCTTGGATGGAAGAAGTTATCAAACTGTGCGGTCGTGAACTGG |
| Relaxin2 (single) | | 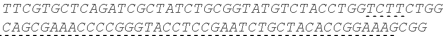 |
| | | 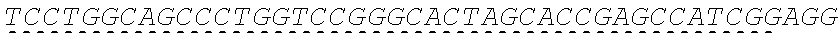 |
| | | 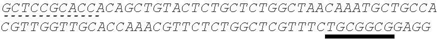 |
| | | 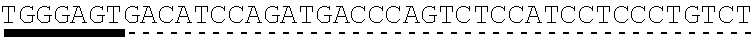 |
| | | 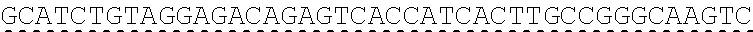 |
| | | 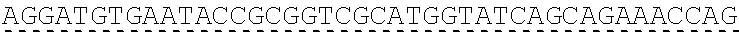 |
| | | 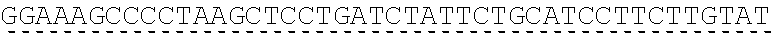 |
| | | 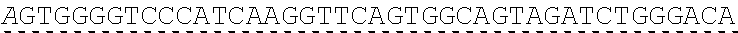 |
| | | 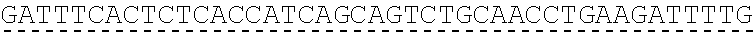 |
| | | 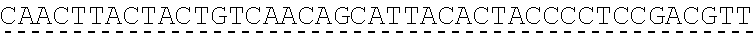 |
| | | 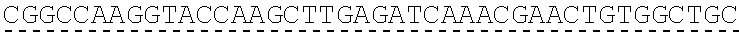 |
| | | 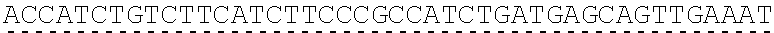 |
| | | 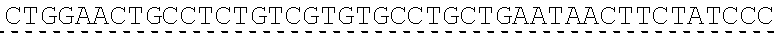 |
| | | 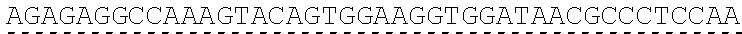 |
| | | 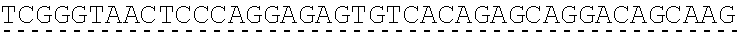 |
| | | 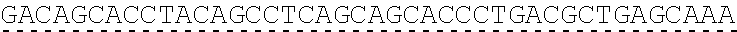 |
| | | 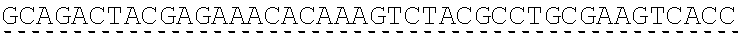 |
| | | 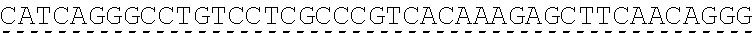 |
| | | 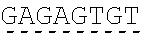 |
| Trastuzumab (NL, GGGGG) | 27 | GACTCTTGGATGGAAGAAGTTATCAAACTGTGCGGTCGTGAACTGG |
| Relaxin2 (single) | | 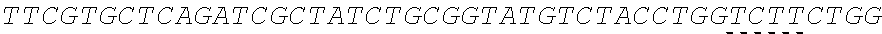 |
| | | 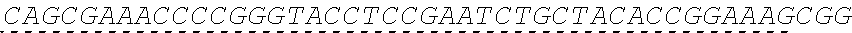 |
| | | 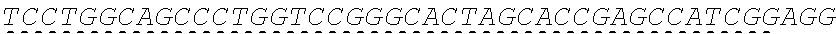 |
| | | 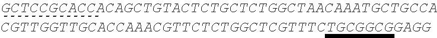 |
| | | 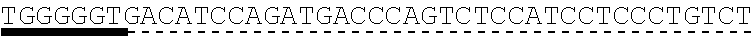 |
| | | 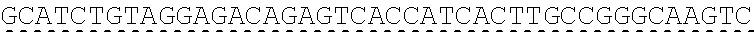 |
| | | 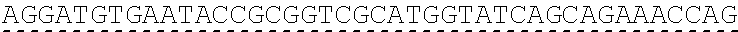 |
| | | 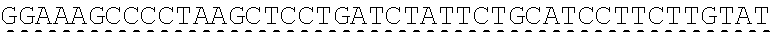 |
| | | 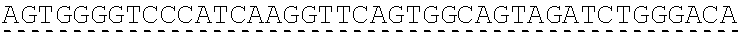 |
| | | 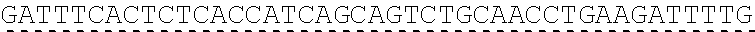 |
| | | 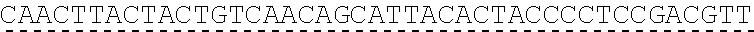 |
| | | 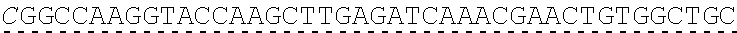 |
| | | 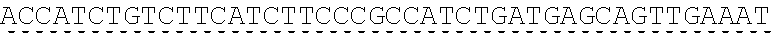 |
| | | 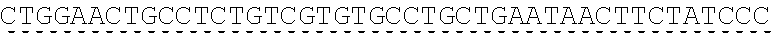 |
| | | 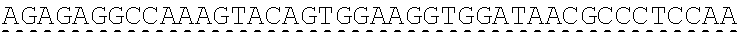 |
| | | 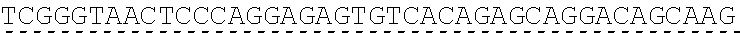 |
| | | 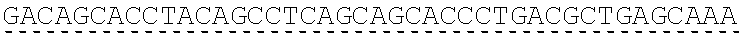 |
| | | 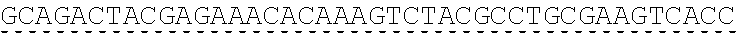 |
| | | 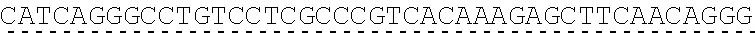 |
| | | 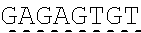 |
| Trastuzumab (NL, CEXGGGGG) | 28 | GACTCTTGGATGGAAGAAGTTATCAAACTGTGCGGTCGTGAACTGG |
| Relaxin2 (single) | | 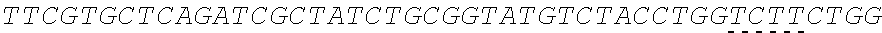 |
| | | 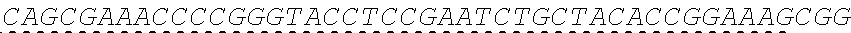 |
| | | 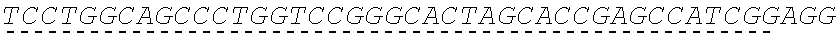 |
| | | 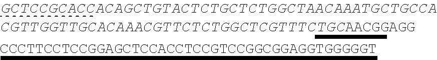 |
| | | 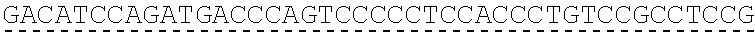 |
| | | 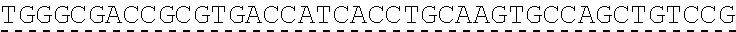 |
| | | 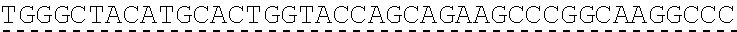 |
| | | 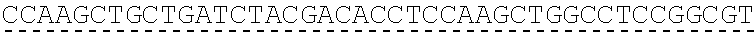 |
| | | 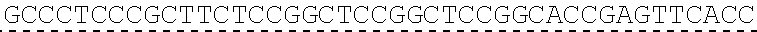 |
| | | 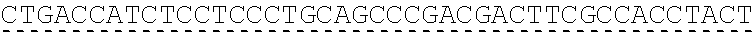 |
| | | 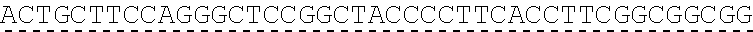 |
| | | 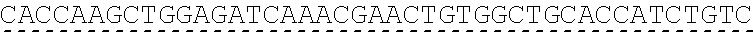 |
| | | 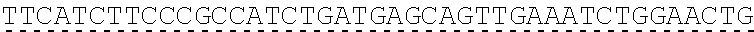 |
| | | 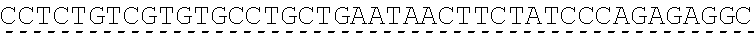 |
| | | 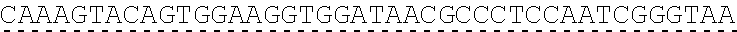 |
| | | 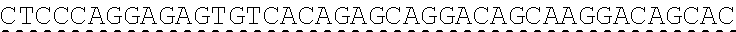 |
| | | 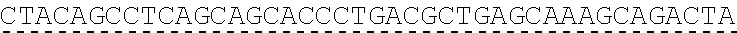 |
| | | 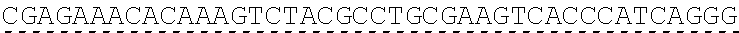 |
| | | 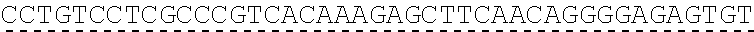 |
| Palivizumab (NL, GGGGS) | 29 | GACTCTTGGATGGAAGAAGTTATCAAACTGTGCGGTCGTGAACTGG |
| Relaxin2 (single) | | 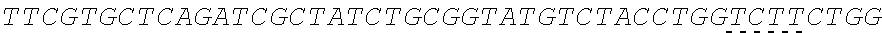 |
| | | 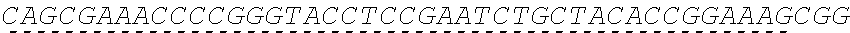 |
| | | 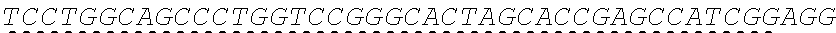 |
| | | 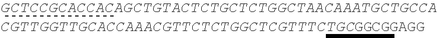 |
| | | 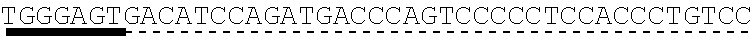 |
| | | 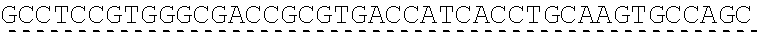 |
| | | 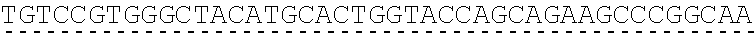 |
| | | 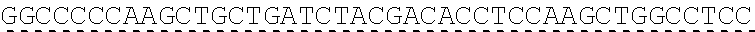 |
| | | 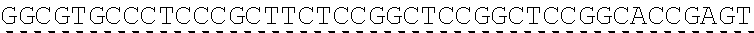 |
| | | 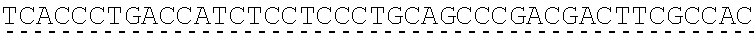 |
| | | 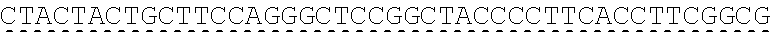 |
| | | 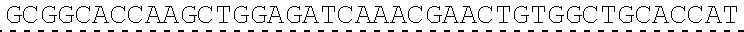 |
| | | 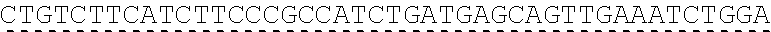 |
| | | 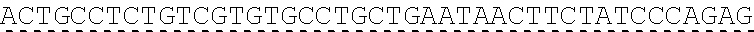 |
| | | 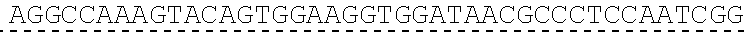 |
| | | 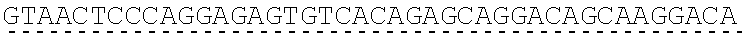 |
| | | 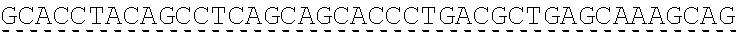 |
| | | 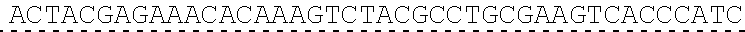 |
| | | 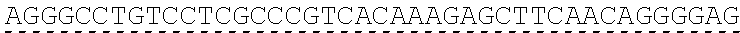 |
| | | 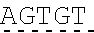 |
| Palivizumab (NL, GGGGG) | 30 | GACTCTTGGATGGAAGAAGTTATCAAACTGTGCGGTCGTGAACTGG |
| Relaxin2 (single) | | 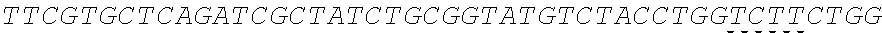 |
| | | 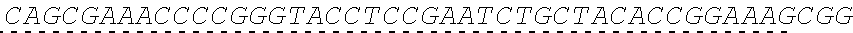 |
| | | 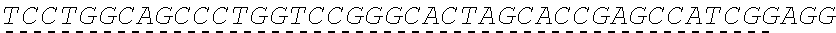 |
| | | 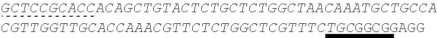 |
| | | 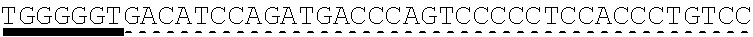 |
| | | 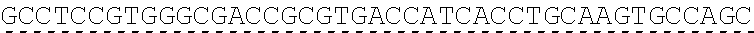 |
| | | 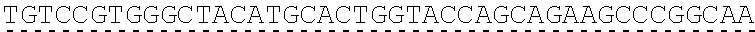 |
| | | 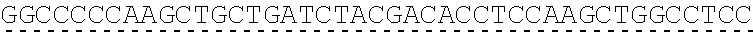 |
| | | 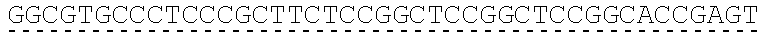 |
| | | 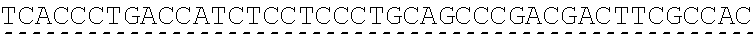 |
| | | 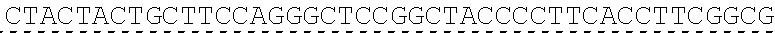 |
| | | 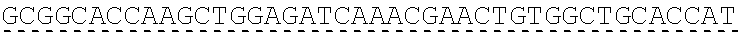 |
| | | 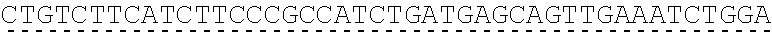 |
| | | 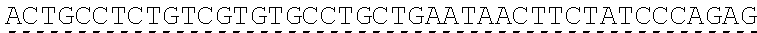 |
| | | 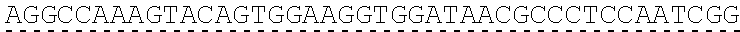 |
| | | 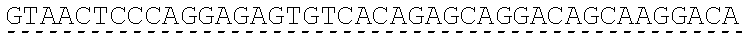 |
| | | 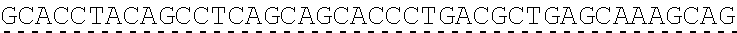 |
| | | 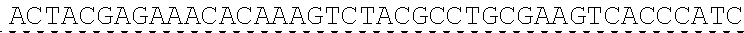 |
| | | 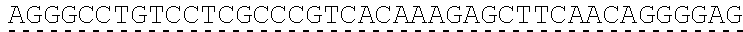 |
| | | 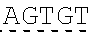 |
| Palivizumab (NL, CEXGGGGG) | 31 | GACTCTTGGATGGAAGAAGTTATCAAACTGTGCGGTCGTGAACTGG |
| Relaxin2 (single) | | 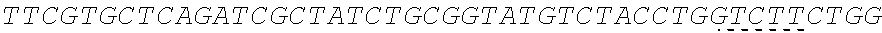 |
| | | 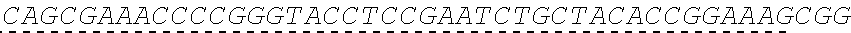 |
| | | 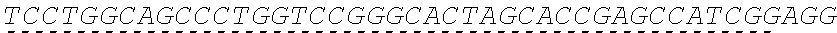 |
| | | 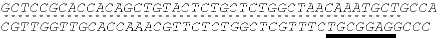 |
| | | 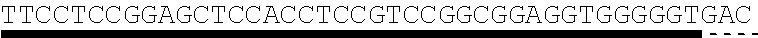 |
| | | 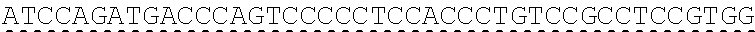 |
| | | 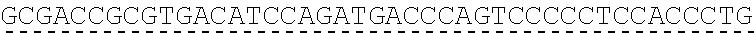 |
| | | 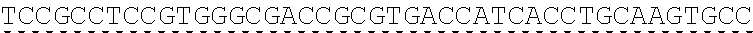 |
| | | 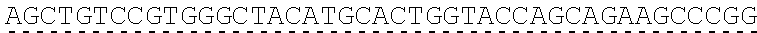 |
| | | 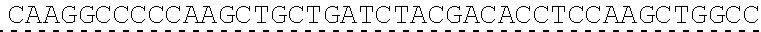 |
| | | 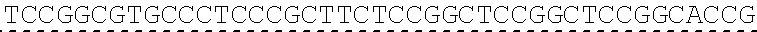 |
| | | 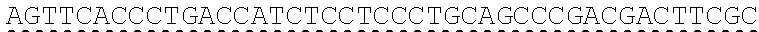 |
| | | 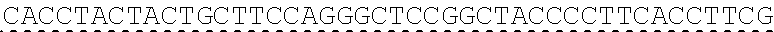 |
| | | 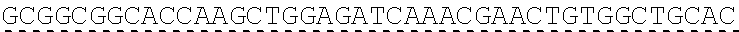 |
| | | 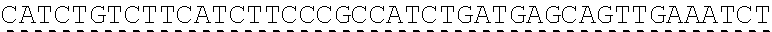 |
| | | 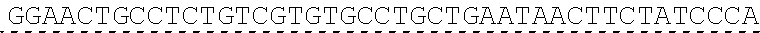 |
| | | 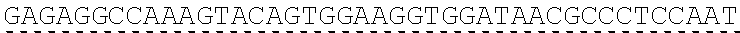 |
| | | 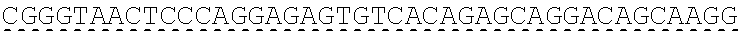 |
| | | 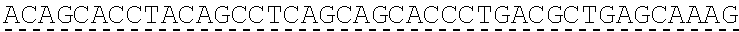 |
| | | 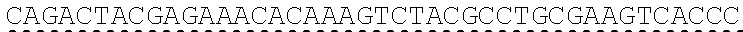 |
| | | 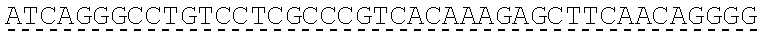 |
| | | 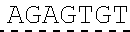 |
| Trastuzumab (NL) Re1axin2 | 32 | GACTCTTGGATGGAAGAAGTTATCAAACTGTGCGGTCGTGAACTGG |
| (insulin C peptide) | | 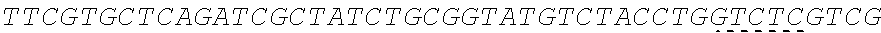 |
| | | 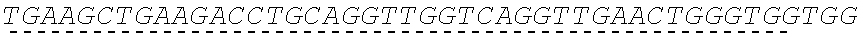 |
| | | 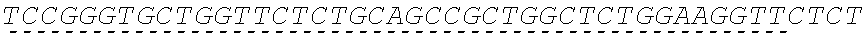 |
| | | 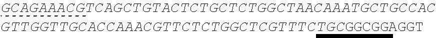 |
| | | 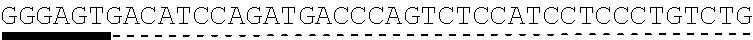 |
| | | 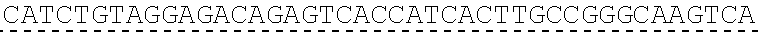 |
| | | 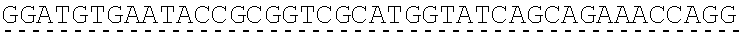 |
| | | 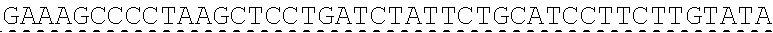 |
| | | 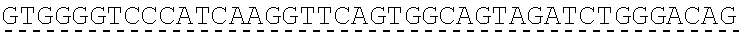 |
| | | 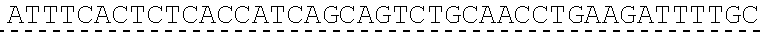 |
| | | 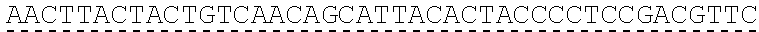 |
| | | 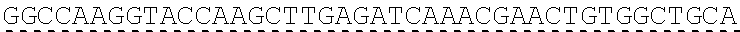 |
| | | 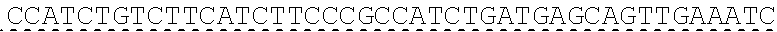 |
| | | 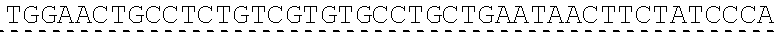 |
| | | 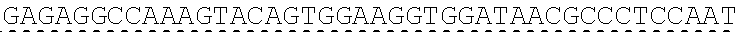 |
| | | 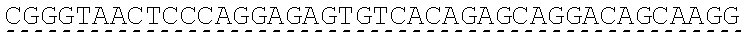 |
| | | 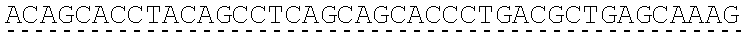 |
| | | 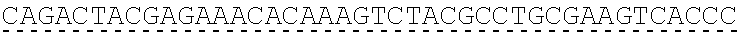 |
| | | 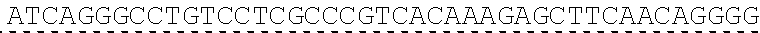 |
| | | 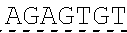 |
| Trastuzumab (NL, GGGGS) | 33 | GACTCTTGGATGGAAGAAGTTATCAAACTGTGCGGTCGTGAACTGG |
| Relaxin2 (XT21) | | 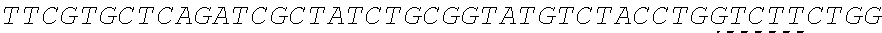 |
| | | 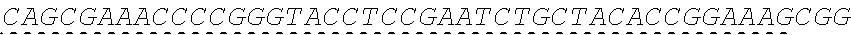 |
| | | 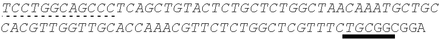 |
| | | 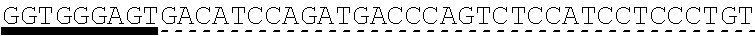 |
| | | 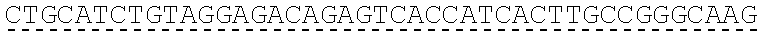 |
| | | 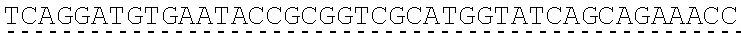 |
| | | 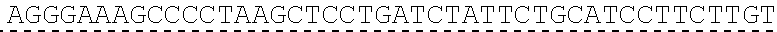 |
| | | 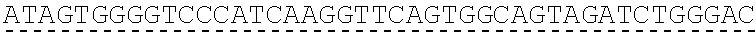 |
| | | 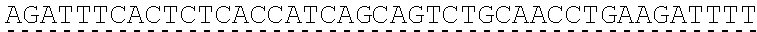 |
| | | 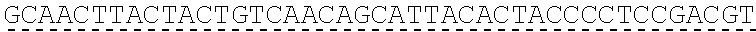 |
| | | 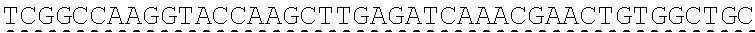 |
| | | 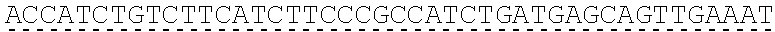 |
| | | 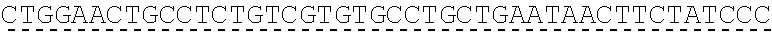 |
| | | 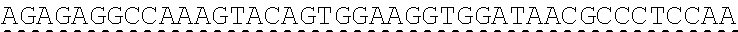 |
| | | 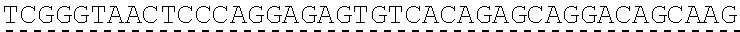 |
| | | 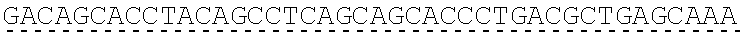 |
| | | 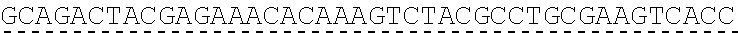 |
| | | 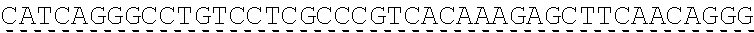 |
| | | 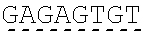 |
| Trastuzumab (NL, GGGGS) | 34 | TTTGTGAACCAACACCTGTGCGGCTCAGACCTGGTGGAAGCTCTCT |
| Insulin | | ACCTAGTGTGCGGGGAACGAGGCTTCTTCTACACAGACCCCACCG |
| | | GCGGAGGGCCCCGCCGGGGCATTGTGGAACAATGCTGTCACAGC |
| | | 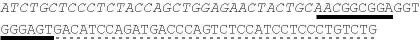 |
| | | 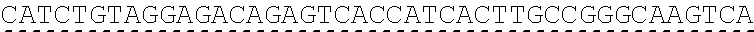 |
| | | 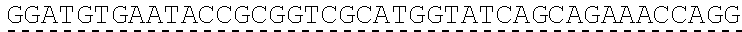 |
| | | 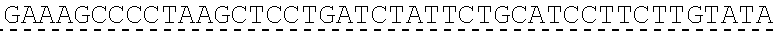 |
| | | 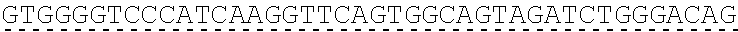 |
| | | 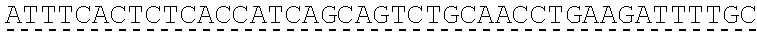 |
| | | 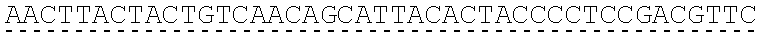 |
| | | 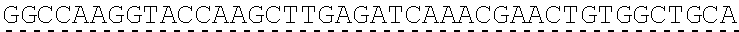 |
| | | 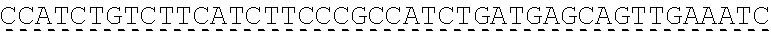 |
| | | 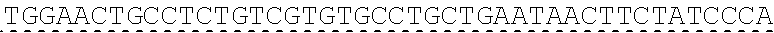 |
| | | 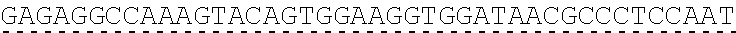 |
| | | 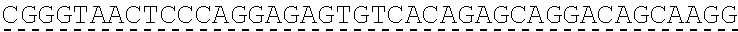 |
| | | 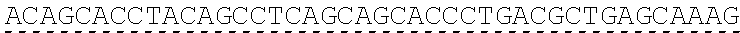 |
| | | 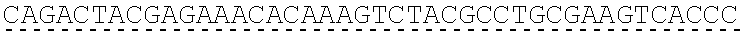 |
| | | 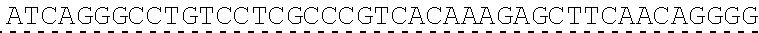 |
| | | 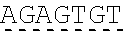 |
| Trastuzumab (NL, GGGGS) | 35 | CATAGCCAGGGAACCTTCACCTCCGACTACAGCAAATACCTTGACA |
| Oxyntomodulin | | GTAGGAGAGCTCAGGATTTTGTGCAATGGCTGATGAACACAAAGAG |
| | | 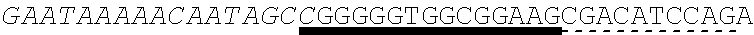 |
| | | 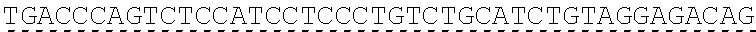 |
| | | 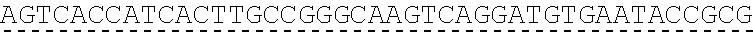 |
| | | 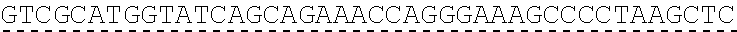 |
| | | 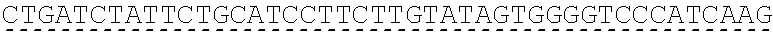 |
| | | 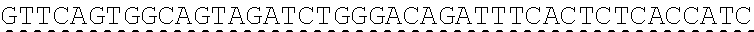 |
| | | 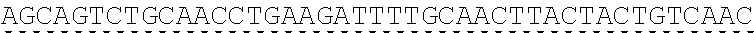 |
| | | 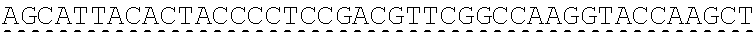 |
| | | 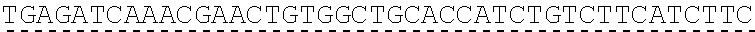 |
| | | 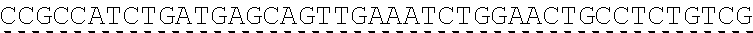 |
| | | 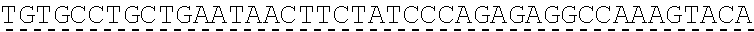 |
| | | 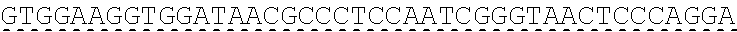 |
| | | 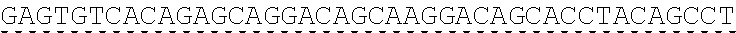 |
| | | 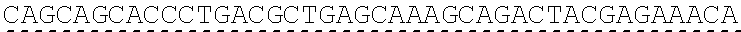 |
| | | 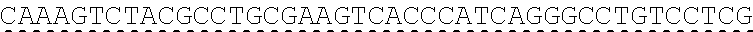 |
| | | 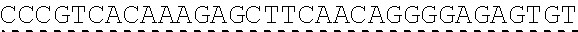 |
| Palivizumab (NL, GGGGS) GLP2 | 36 | CACGGCGACGGTTCATTCTCTGACGAAATGAATACAATACTCGACA |
| | | ACCTCGCCGCCAGGGACTTTATCAATTGGCTCATTCAAACTAAAAT |
| | | 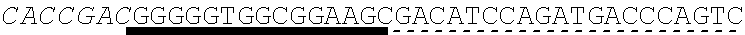 |
| | | 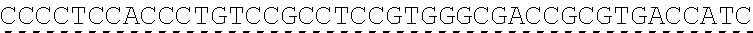 |
| | | 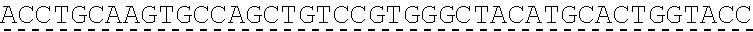 |
| | | 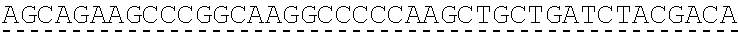 |
| | | 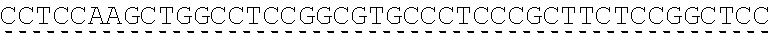 |
| | | 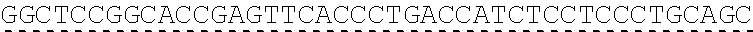 |
| | | 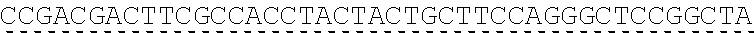 |
| | | 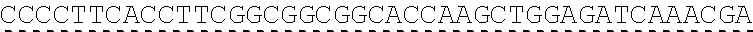 |
| | | 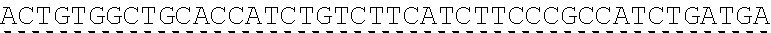 |
| | | 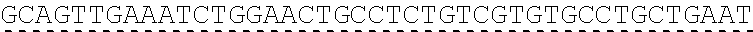 |
| | | 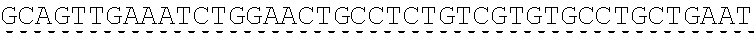 |
| | | 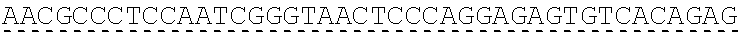 |
| | | 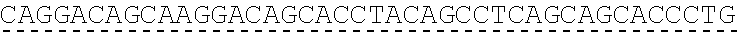 |
| | | 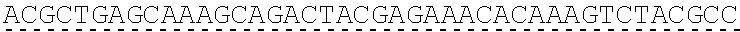 |
| | | 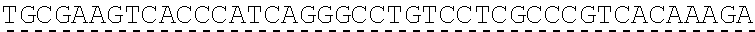 |
| | | 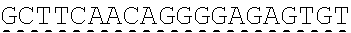 |
| Palivizumab (NL, CEXGGGGS) | 37 | CACGGCGACGGTTCATTCTCTGACGAAATGAATACAATACTCGACA |
| GLP2 | | ACCTCGCCGCCAGGGACTTTATCAATTGGCTCATTCAAACTAAAAT |
| | | 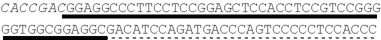 |
| | | 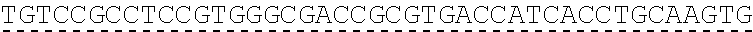 |
| | | 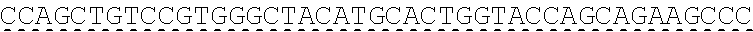 |
| | | 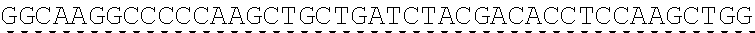 |
| | | 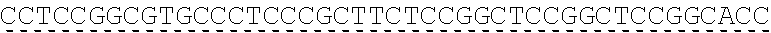 |
| | | 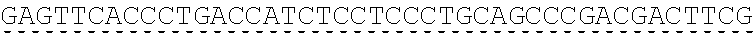 |
| | | 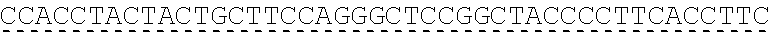 |
| | | 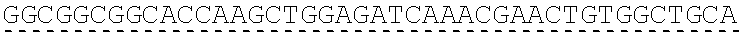 |
| | | 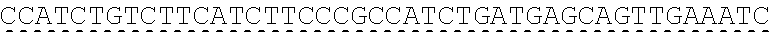 |
| | | 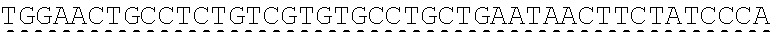 |
| | | 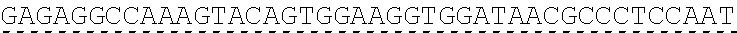 |
| | | 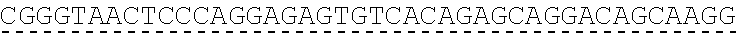 |
| | | 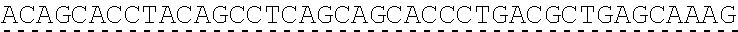 |
| | | 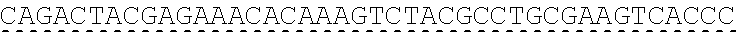 |
| | | 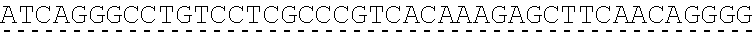 |
| | | 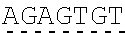 |
| Trastuzumab (NL, GGGGS) Moka | 38 | ATCAACGTGAAGTGCAGCCTGCCCCAGCAGTGCATCAAGCCCTGC |
| | | AAGGACGCCGGCATGCGGTTCGGCAAGTGCATGAACAAGAAGTGC |
| | | 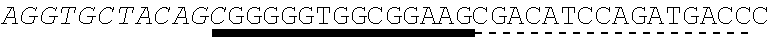 |
| | | 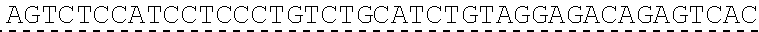 |
| | | 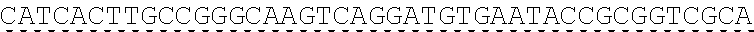 |
| | | 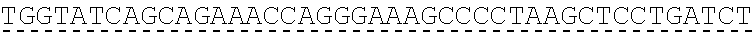 |
| | | 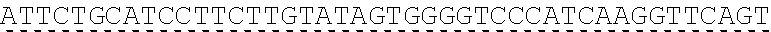 |
| | | 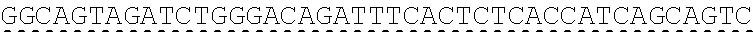 |
| | | 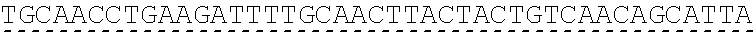 |
| | | 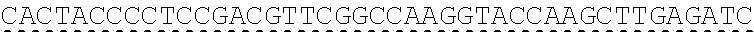 |
| | | 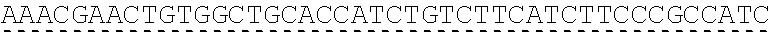 |
| | | 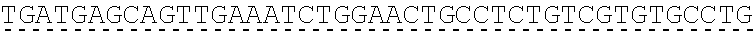 |
| | | 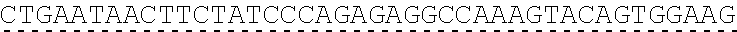 |
| | | 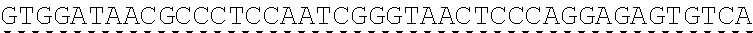 |
| | | 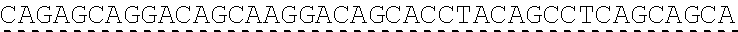 |
| | | 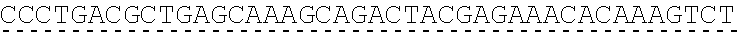 |
| | | 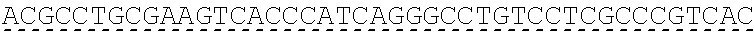 |
| | | 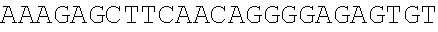 |
| Trastuzumab (NL, GGGGS) | 39 | GCTGACAACAAATGCGAAAACTCTCTGCGTCGTGAAATCGCTTGCG |
| Ssam6 | | GTCAGTGCCGTGACAAAGTTAAAACCGACGGTTACTTCTACGAATG |
| | | CTGCACCTCTGACTCTACCTTCAAAAAATGCCAGGACCTGCTGCAC |
| | | 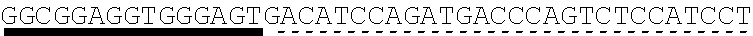 |
| | | 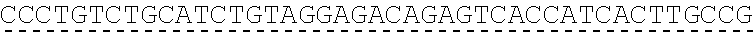 |
| | | 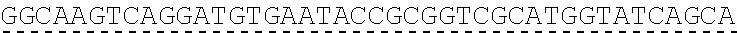 |
| | | 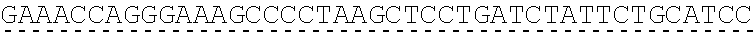 |
| | | 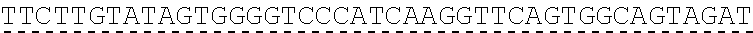 |
| | | 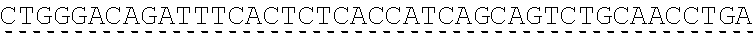 |
| | | 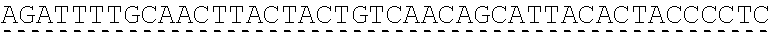 |
| | | 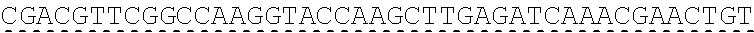 |
| | | 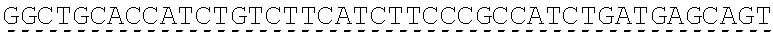 |
| | | 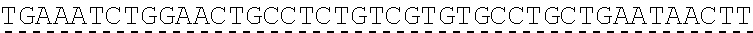 |
| | | 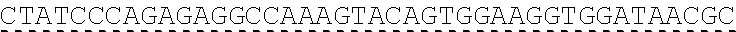 |
| | | 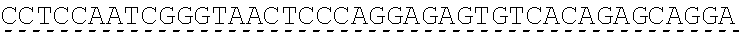 |
| | | 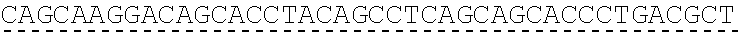 |
| | | 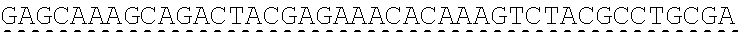 |
| | | 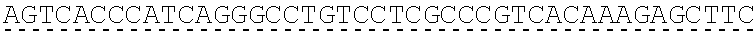 |
| | | 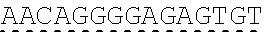 |
| Trastuzumab (NL, GGGGS) | 40 | GAATGCATCGGTATGTTCAAATCTTGCGACCCGGAAAACGACAAAT |
| 550 | | GCTGCAAAGGTCGTACCTGCTCTCGTAAACACCGTTGGTGCAAATA |
| | | 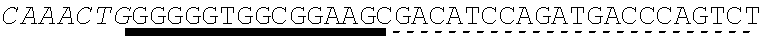 |
| | | 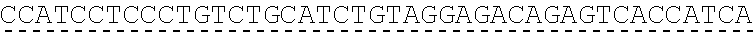 |
| | | 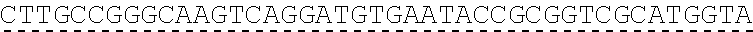 |
| | | 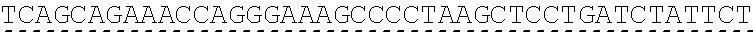 |
| | | 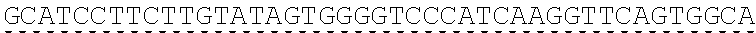 |
| | | 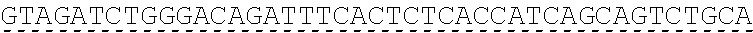 |
| | | 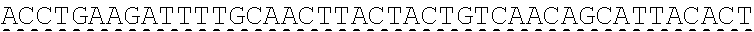 |
| | | 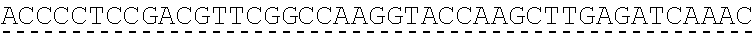 |
| | | 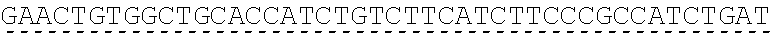 |
| | | 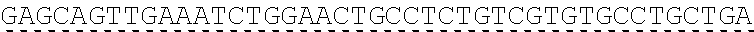 |
| | | 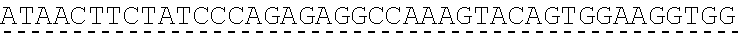 |
| | | 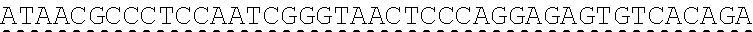 |
| | | 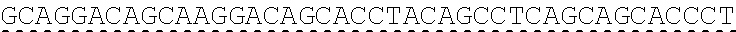 |
| | | 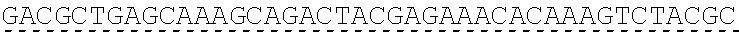 |
| | | 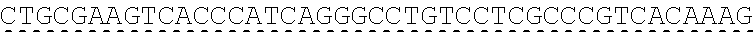 |
| | | 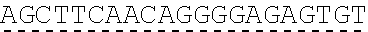 |
| Trastuzumab (NL, GGGGS) | 41 | CTGAAATGTTACCAACATGGTAAAGTTGTGACTTGTCATCGAGATAT |
| Mambalign 1 | | GAAGTTTTGCTATCATAACACTGGCATGCCTTTTCGAAATCTCAAGC |
| | | TCATCCTACAGGGATGTTCTTCTTCGTGCAGTGAAACAGAAAACAAT |
| | | 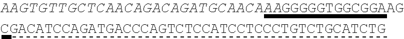 |
| | | 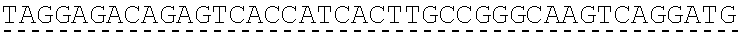 |
| | | 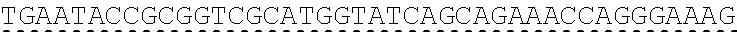 |
| | | 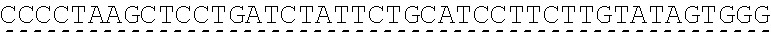 |
| | | 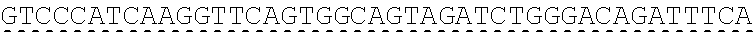 |
| | | 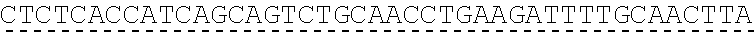 |
| | | 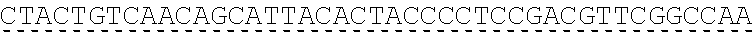 |
| | | 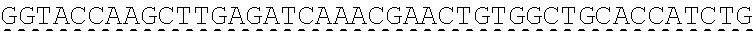 |
| | | 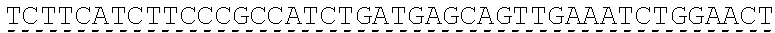 |
| | | 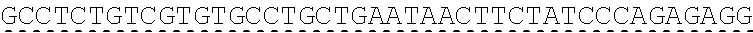 |
| | | 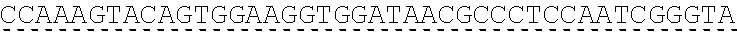 |
| | | 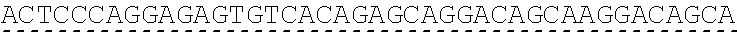 |
| | | 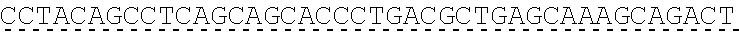 |
| | | 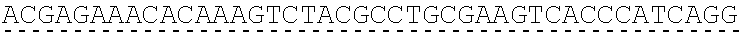 |
| | | 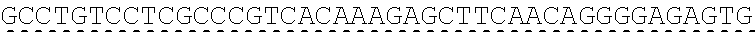 |
| | | 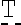 |
| Palivizumab (NH, GGGGS) | 161 | CACGGAGAAGGAACATTTACCAGCGACCTCAGCAAGCAGATGGAG |
| Exendin-4 | | GAAGAGGCCGTGAGGCTGTTCATCGAGTGGCTGAAGAACGGCGG |
| | | 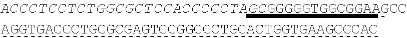 |
| | | 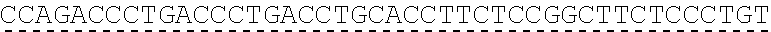 |
| | | 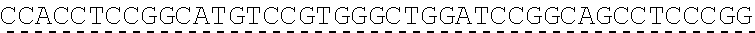 |
| | | 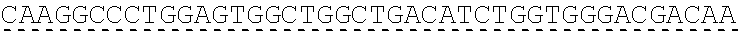 |
| | | 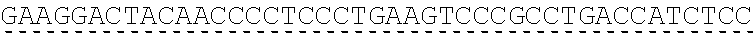 |
| | | 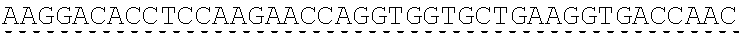 |
| | | 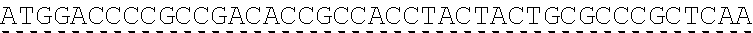 |
| | | 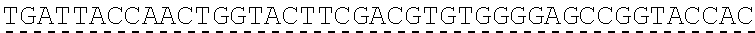 |
| | | 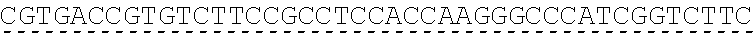 |
| | | 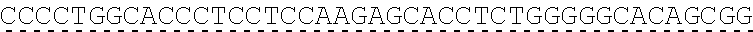 |
| | | 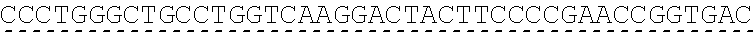 |
| | | 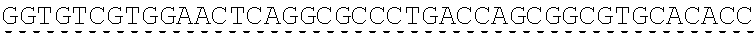 |
| | | 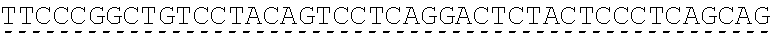 |
| | | 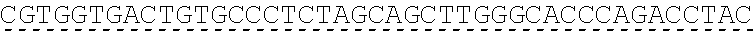 |
| | | 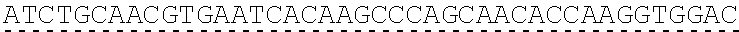 |
| | | 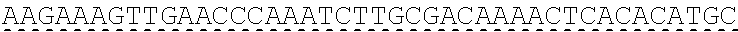 |
| | | 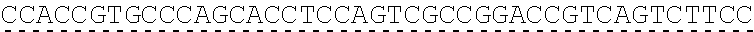 |
| | | 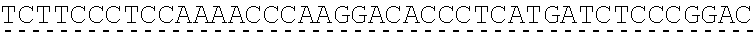 |
| | | 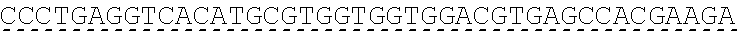 |
| | | 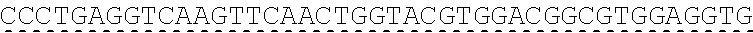 |
| | | 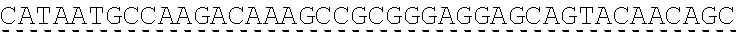 |
| | | 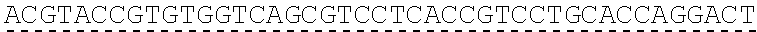 |
| | | 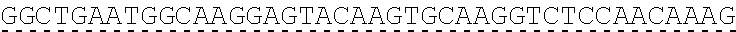 |
| | | 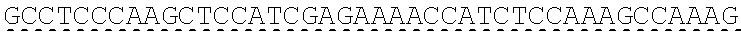 |
| | | 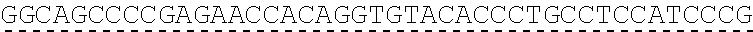 |
| | | 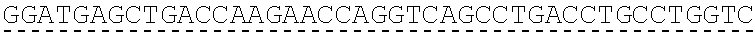 |
| | | 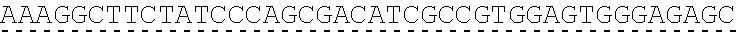 |
| | | 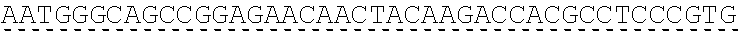 |
| | | 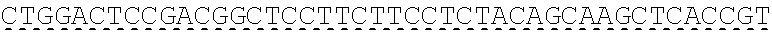 |
| | | 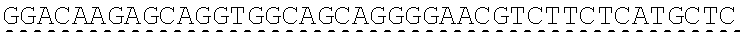 |
| | | 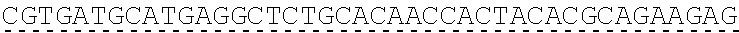 |
| | | 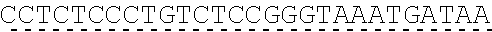 |
| Palivizumab (NL, EAAAK) | 162 | CATTCACAGGGCACATTCACCAGTGACTACAGCAAGTATCTGGACT |
| Glucagon | | 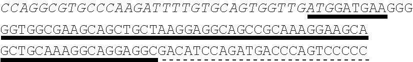 |
| | | 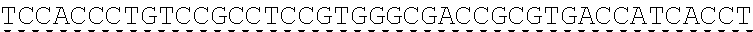 |
| | | 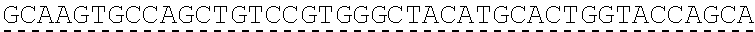 |
| | | 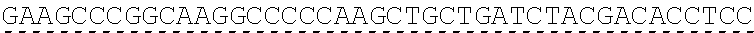 |
| | | 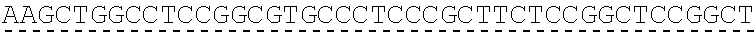 |
| | | 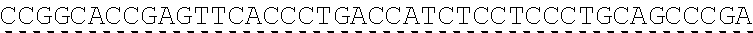 |
| | | 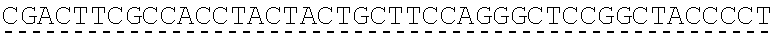 |
| | | 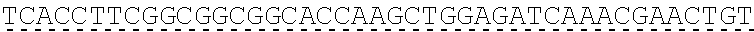 |
| | | 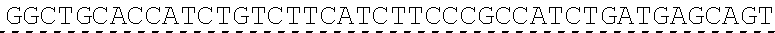 |
| | | 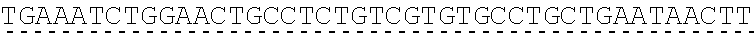 |
| | | 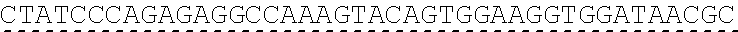 |
| | | 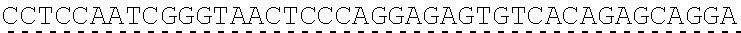 |
| | | 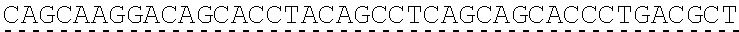 |
| | | 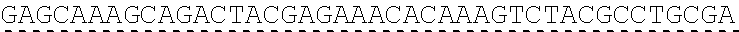 |
| | | 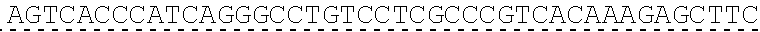 |
| | | 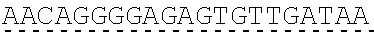 |
| Palivizumab (NL, GGGGG) | 163 | CATTCACAGGGCACATTCACCAGTGACTACAGCAAGTATCTGGACT |
| Glucagon | | 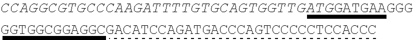 |
| | | 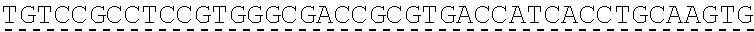 |
| | | 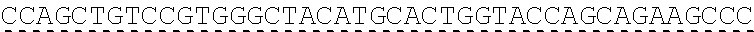 |
| | | 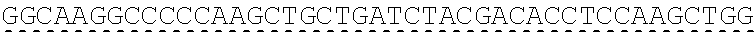 |
| | | 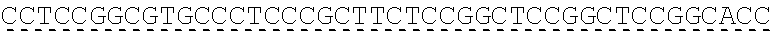 |
| | | 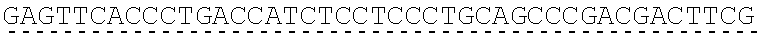 |
| | | 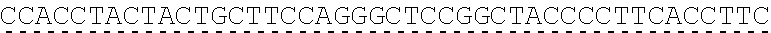 |
| | | 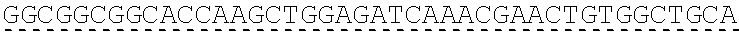 |
| | | 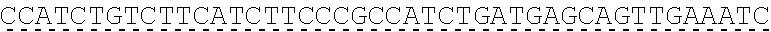 |
| | | 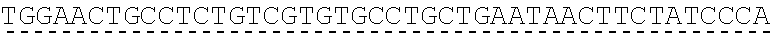 |
| | | 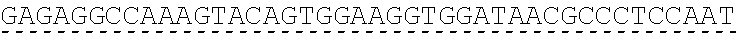 |
| | | 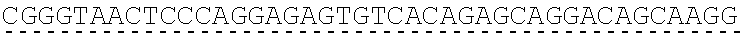 |
| | | 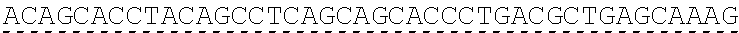 |
| | | 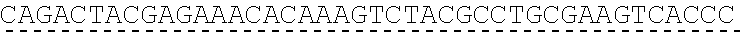 |
| | | 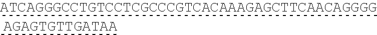 |
| Palivizumab (NH, EAAAK) | 164 | CATTCACAGGGCACATTCACCAGTGACTACAGCAAGTATCTGGACT |
| Glucagon | | 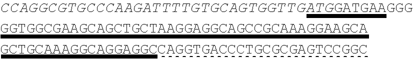 |
| | | 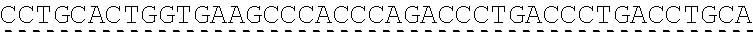 |
| | | 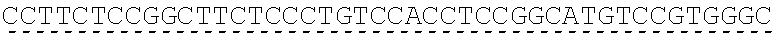 |
| | | 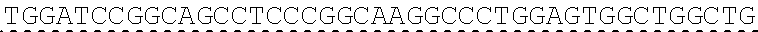 |
| | | 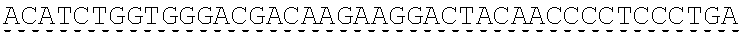 |
| | | 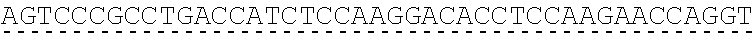 |
| | | 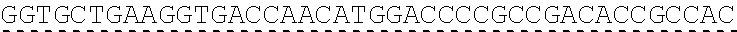 |
| | | 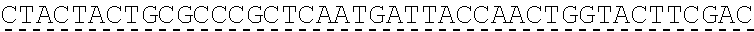 |
| | | 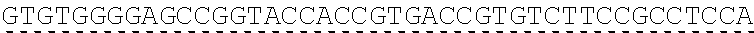 |
| | | 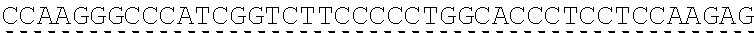 |
| | | 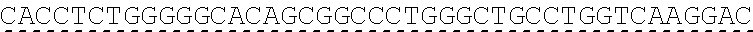 |
| | | 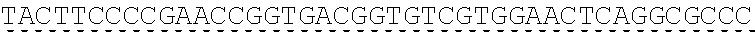 |
| | | 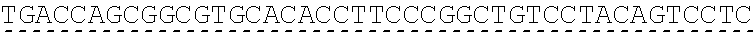 |
| | | 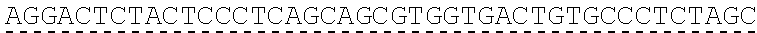 |
| | | 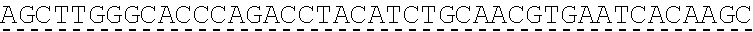 |
| | | 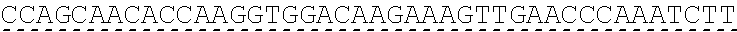 |
| | | 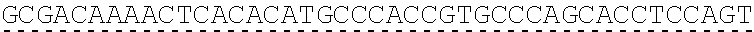 |
| | | 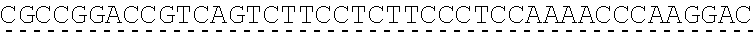 |
| | | 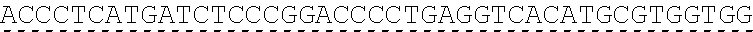 |
| | | 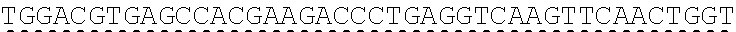 |
| | | 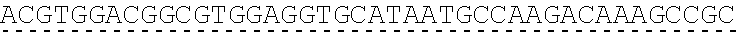 |
| | | 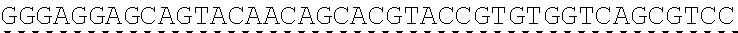 |
| | | 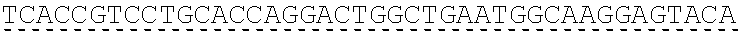 |
| | | 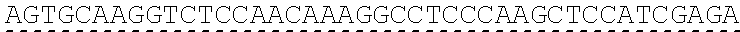 |
| | | 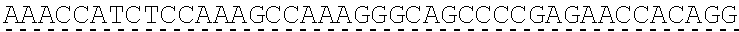 |
| | | 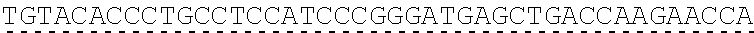 |
| | | 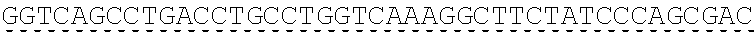 |
| | | 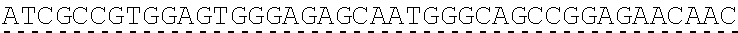 |
| | | 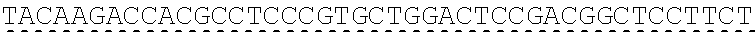 |
| | | 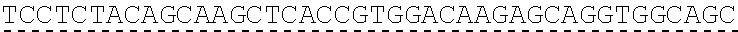 |
| | | 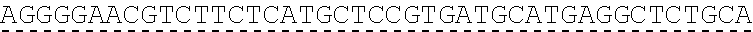 |
| | | 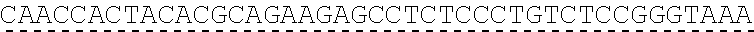 |
| | | 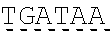 |
| Palivizumab (NH, EAAAK) ZP1 | 165 | CACGGCCAGGGCACATTCACTAGCGATTATAGTAAATATCTGGATT |
| | | 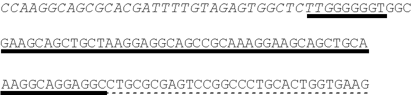 |
| | | 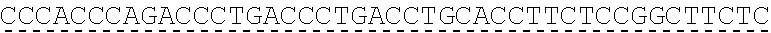 |
| | | 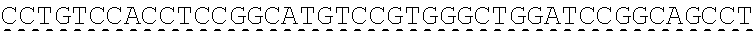 |
| | | 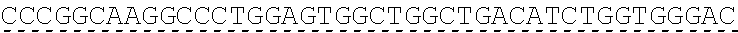 |
| | | 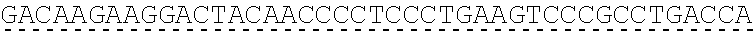 |
| | | 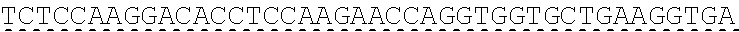 |
| | | 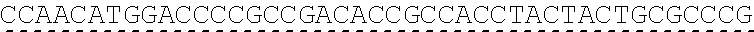 |
| | | 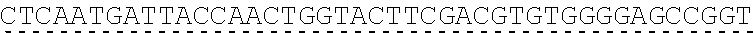 |
| | | 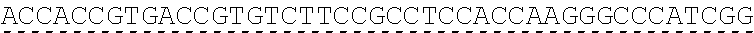 |
| | | 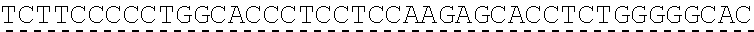 |
| | | 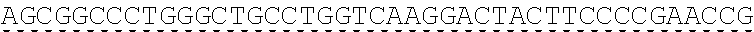 |
| | | 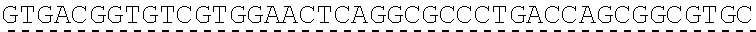 |
| | | 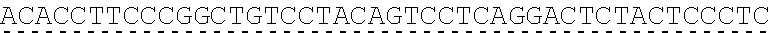 |
| | | 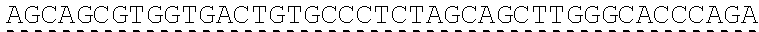 |
| | | 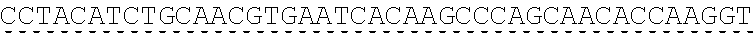 |
| | | 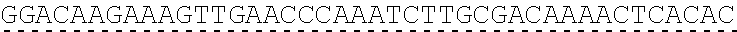 |
| | | 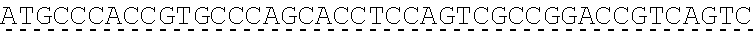 |
| | | 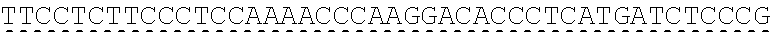 |
| | | 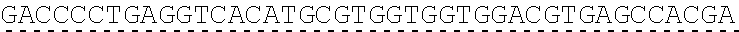 |
| | | 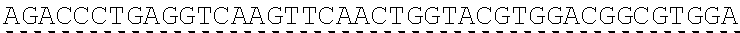 |
| | | 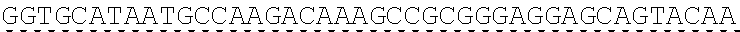 |
| | | 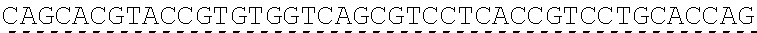 |
| | | 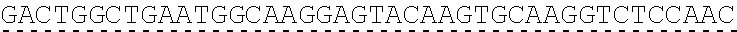 |
| | | 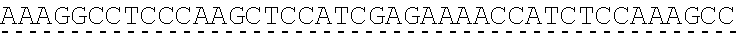 |
| | | 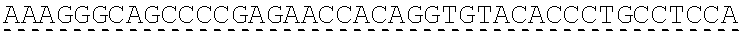 |
| | | 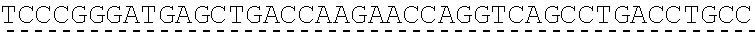 |
| | | 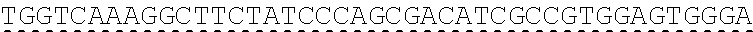 |
| | | 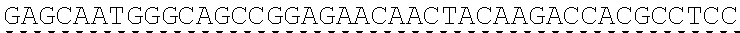 |
| | | 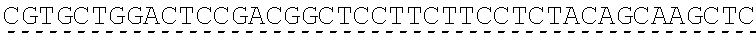 |
| | | 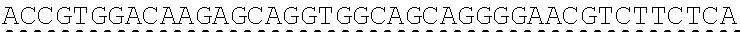 |
| | | 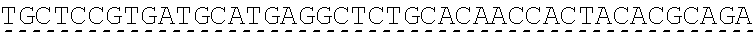 |
| Trastuzumab (NH, EAAAK) ZP1 | 166 | CACGGCCAGGGCACATTCACTAGCGATTATAGTAAATATCTGGATT |
| | | 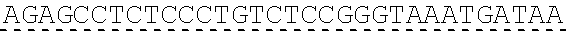 |
| | | 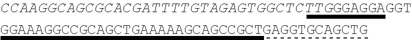 |
| | | 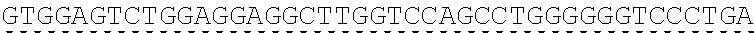 |
| | | 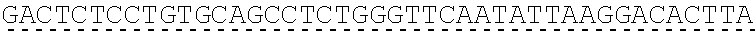 |
| | | 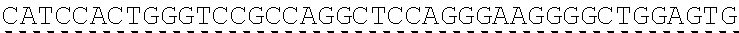 |
| | | 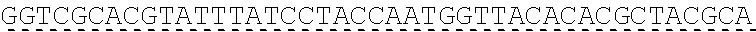 |
| | | 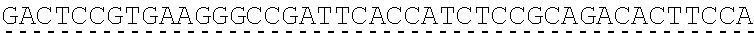 |
| | | 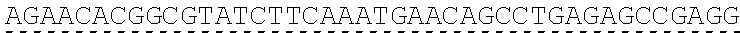 |
| | | 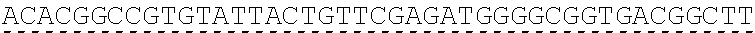 |
| | | 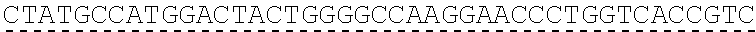 |
| | | 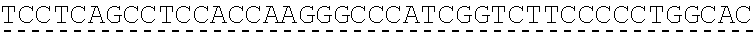 |
| | | 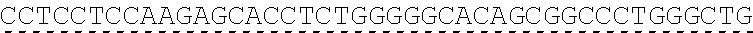 |
| | | 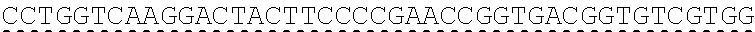 |
| | | 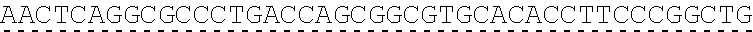 |
| | | 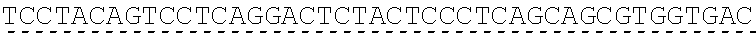 |
| | | 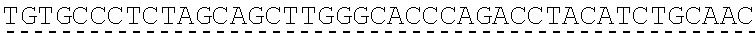 |
| | | 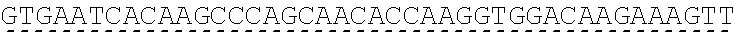 |
| | | 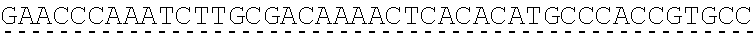 |
| | | 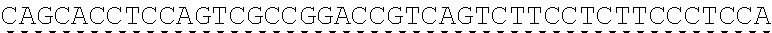 |
| | | 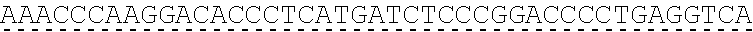 |
| | | 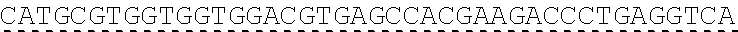 |
| | | 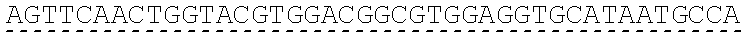 |
| | | 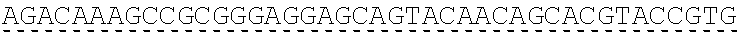 |
| | | 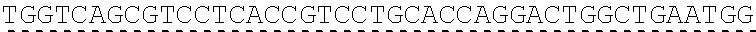 |
| | | 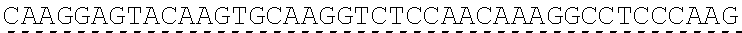 |
| | | 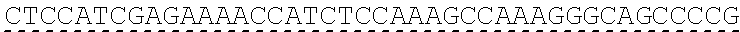 |
| | | 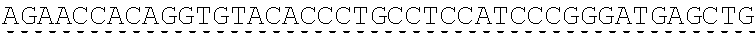 |
| | | 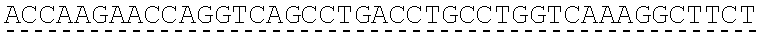 |
| | | 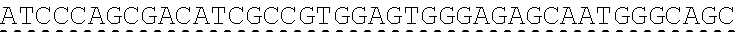 |
| | | 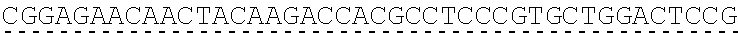 |
| | | 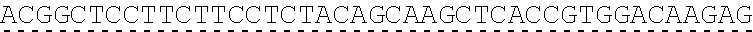 |
| | | 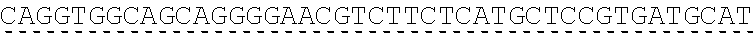 |
| | | 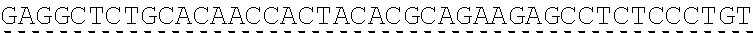 |
| | | 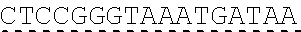 |
| Trastuzumab (NH, XT21) ZP1 | 167 | CACGGCCAGGGCACATTCACTAGCGATTATAGTAAATATCTGGATT |
| | | 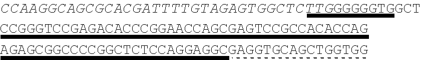 |
| | | 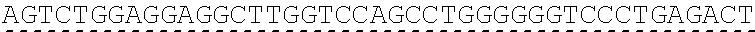 |
| | | 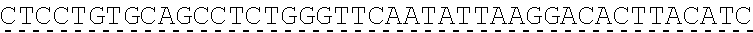 |
| | | 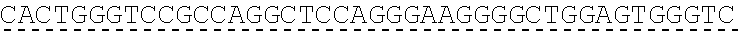 |
| | | 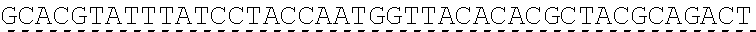 |
| | | 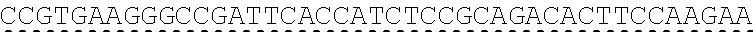 |
| | | 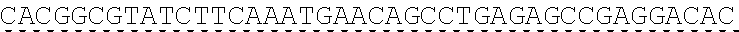 |
| | | 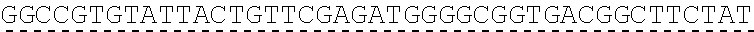 |
| | | 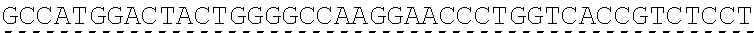 |
| | | 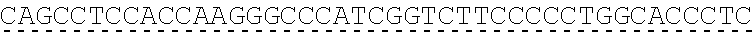 |
| | | 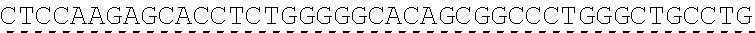 |
| | | 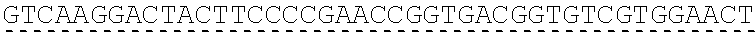 |
| | | 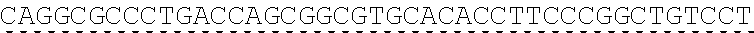 |
| | | 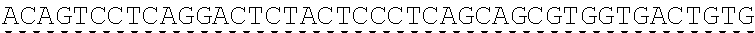 |
| | | 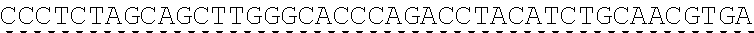 |
| | | 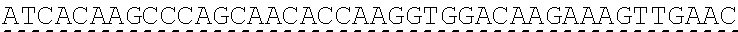 |
| | | 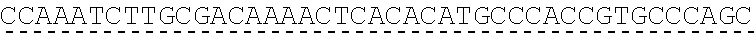 |
| | | 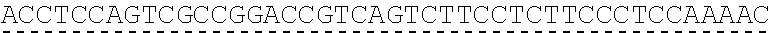 |
| | | 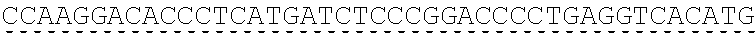 |
| | | 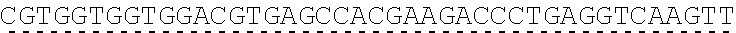 |
| | | 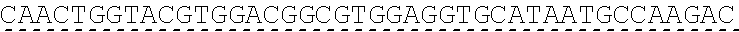 |
| | | 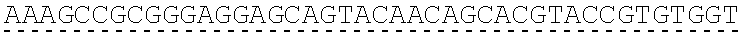 |
| | | 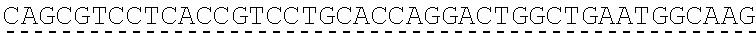 |
| | | 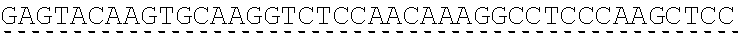 |
| | | 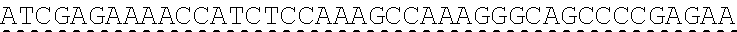 |
| | | 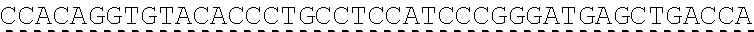 |
| | | 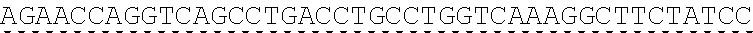 |
| | | 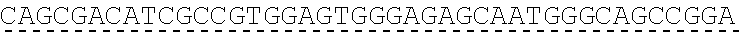 |
| | | 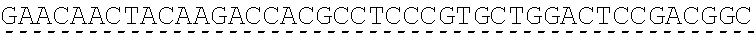 |
| | | 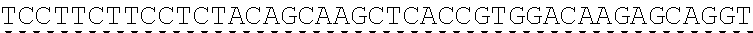 |
| | | 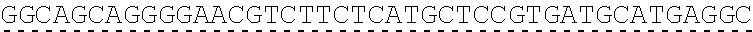 |
| | | 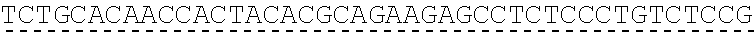 |
| | | 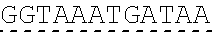 |
| Palivizumab (NL, EAAAK) ZP1 | 168 | CACGGCCAGGGCACATTCACTAGCGATTATAGTAAATATCTGGATT |
| | | 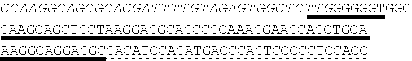 |
| | | 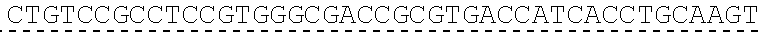 |
| | | 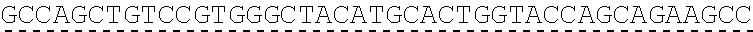 |
| | | 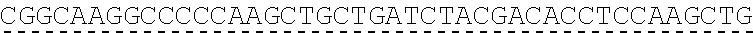 |
| | | 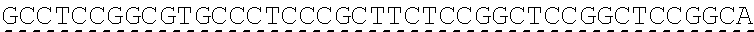 |
| | | 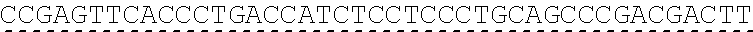 |
| | | 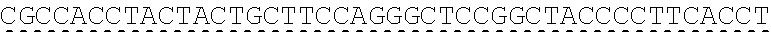 |
| | | 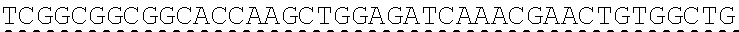 |
| | | 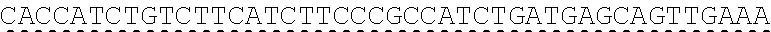 |
| | | 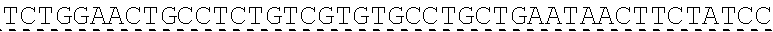 |
| | | 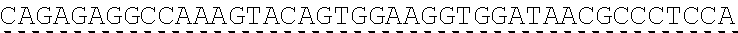 |
| | | 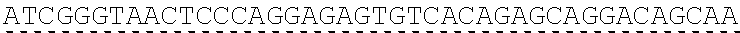 |
| | | 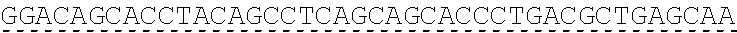 |
| | | 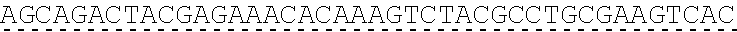 |
| | | 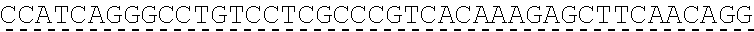 |
| | | 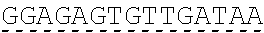 |
| Palivizumab (NL, XT21) ZP1 | 169 | CACAGCCAGGGCACATTCACTAGCGATTATAGTAAATATCTGGATT |
| | | 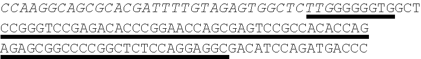 |
| | | 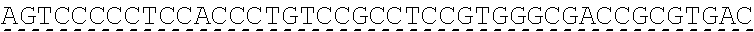 |
| | | 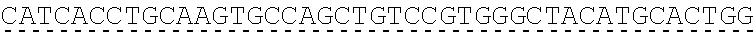 |
| | | 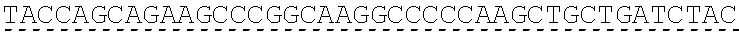 |
| | | 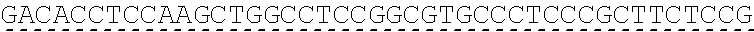 |
| | | 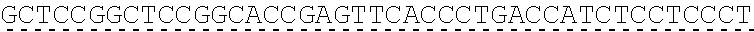 |
| | | 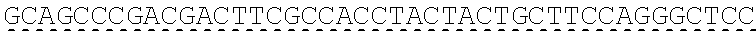 |
| | | 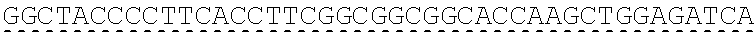 |
| | | 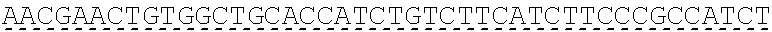 |
| | | 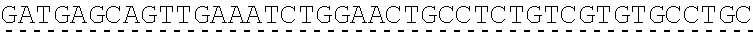 |
| | | 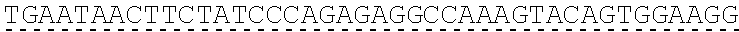 |
| | | 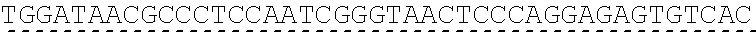 |
| | | 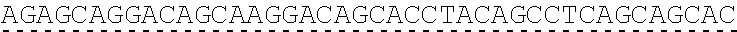 |
| | | 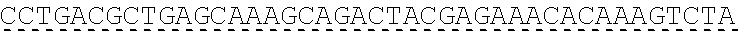 |
| | | 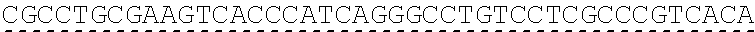 |
| | | 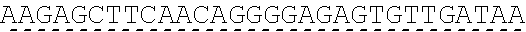 |
| Palivizumab (NH, | 170 | GACTCTTGGATGGAAGAAGTTATCAAACTGTGCGGTCGTGAACTGG |
| CEXGGGGS) Relaxin2 | | 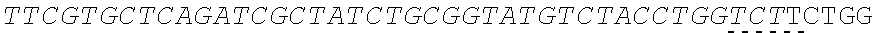 |
| (single) | | 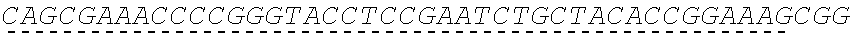 |
| | | 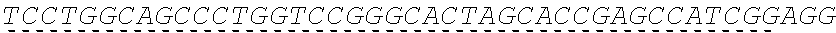 |
| | | 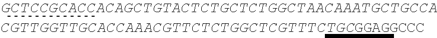 |
| | | 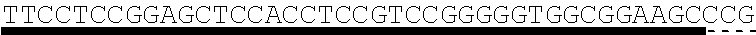 |
| | | 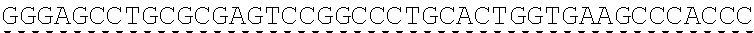 |
| | | 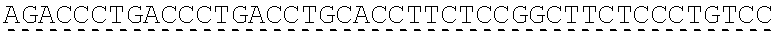 |
| | | 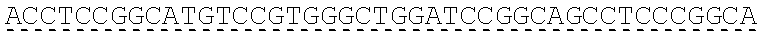 |
| | | 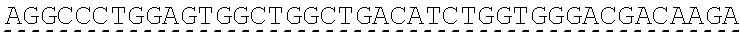 |
| | | 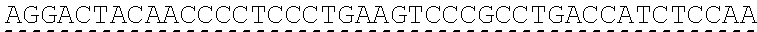 |
| | | 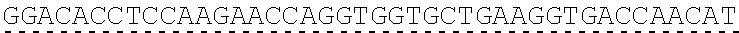 |
| | | 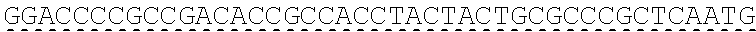 |
| | | 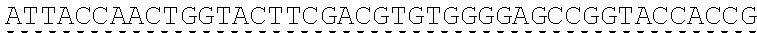 |
| | | 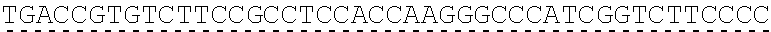 |
| | | 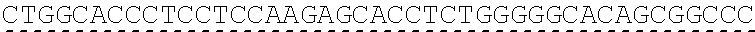 |
| | | 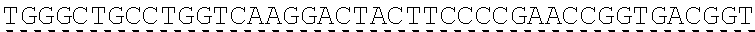 |
| | | 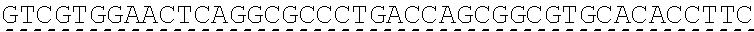 |
| | | 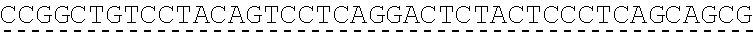 |
| | | 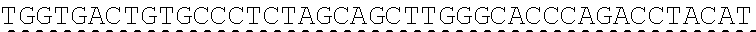 |
| | | 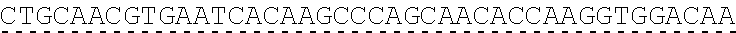 |
| | | 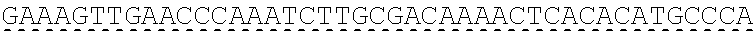 |
| | | 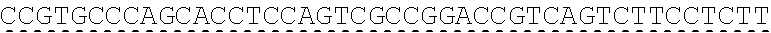 |
| | | 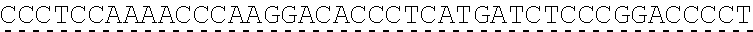 |
| | | 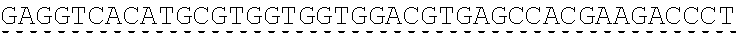 |
| | | 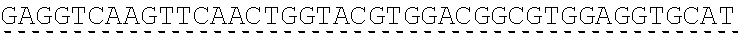 |
| | | 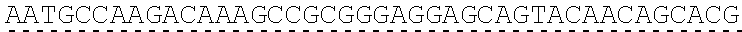 |
| | | 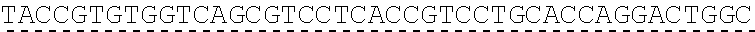 |
| | | 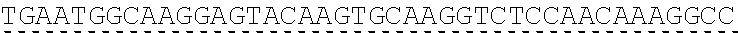 |
| | | 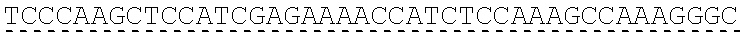 |
| | | 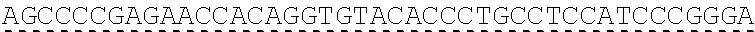 |
| | | 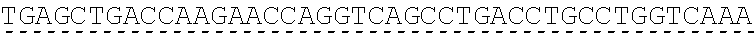 |
| | | 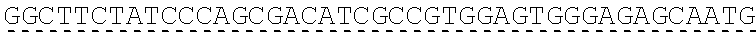 |
| | | 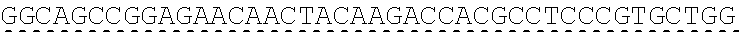 |
| | | 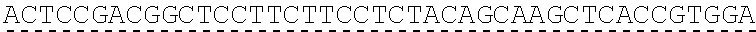 |
| | | 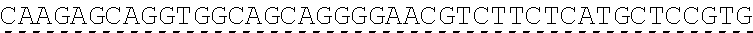 |
| | | 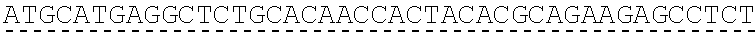 |
| | | 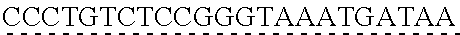 |
| Palivizumab (NH, EAAAK) | 171 | GACTCTTGGATGGAAGAAGTTATCAAACTGTGCGGTCGTGAACTGG |
| Relaxin2 (XT35) | | TTCGTGCTCAGATCGCTATCTGCGGTATGTCTACCTGGTCTAAACG |
| | | 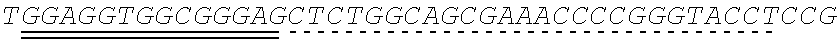 |
| | | 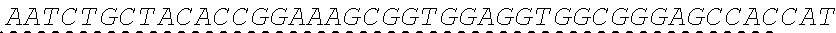 |
| | | 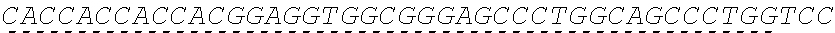 |
| | | 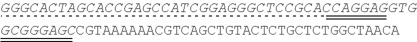 |
| | | AATGCTGCCACGTTGGTTGCACCAAACGTTCTCTGGCTCGTTTCTG |
| | | 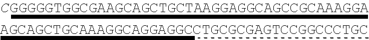 |
| | | 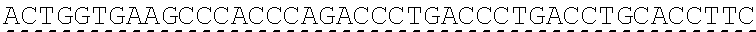 |
| | | 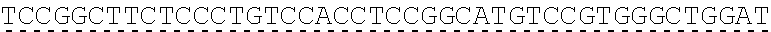 |
| | | 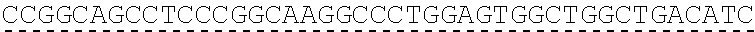 |
| | | 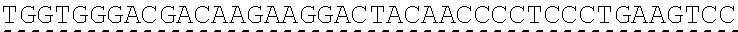 |
| | | 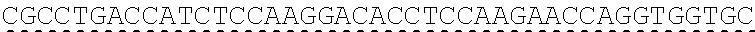 |
| | | 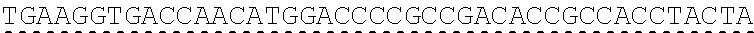 |
| | | 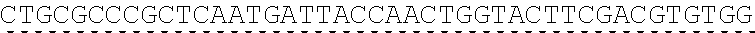 |
| | | 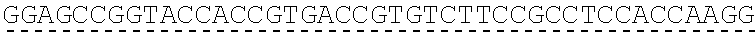 |
| | | 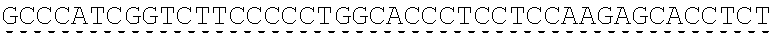 |
| | | 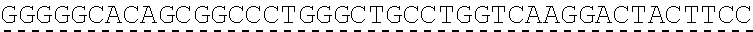 |
| | | 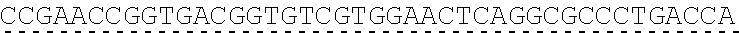 |
| | | 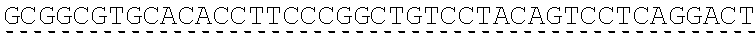 |
| | | 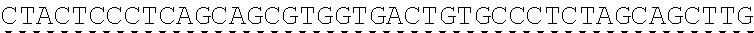 |
| | | 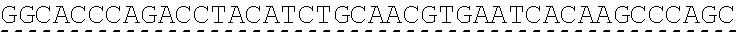 |
| | | 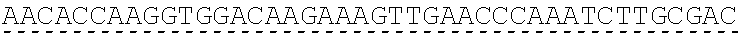 |
| | | 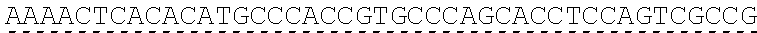 |
| | | 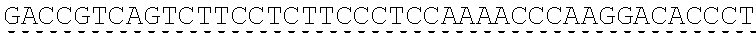 |
| | | 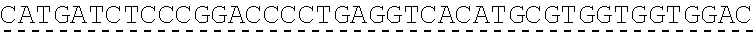 |
| | | 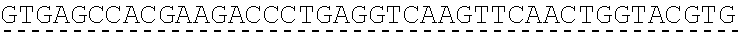 |
| | | 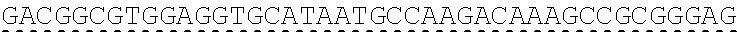 |
| | | 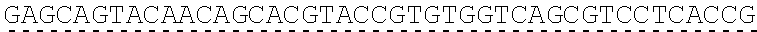 |
| | | 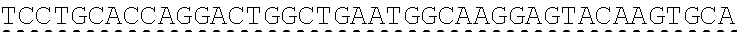 |
| | | 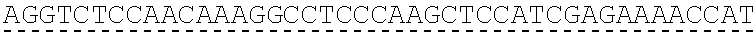 |
| | | 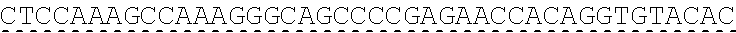 |
| | | 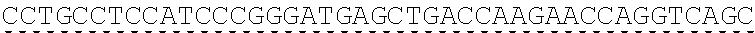 |
| | | 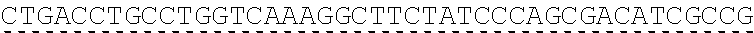 |
| | | 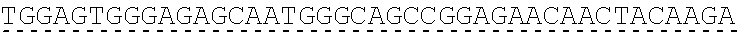 |
| | | 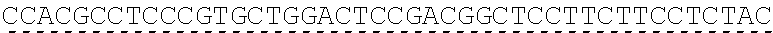 |
| | | 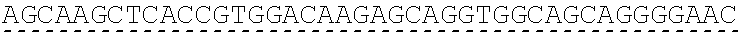 |
| | | 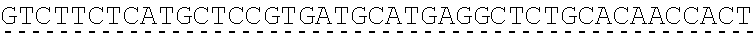 |
| | | 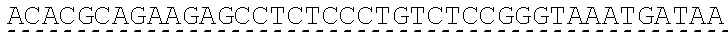 |
| Palivizumab Fab (NH, | 172 | GACTCTTGGATGGAAGAAGTTATCAAACTGTGCGGTCGTGAACTGG |
| CEXGGGGS) Relaxin2 (single) | | 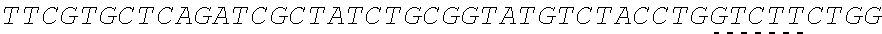 |
| | | 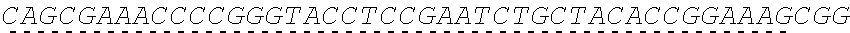 |
| | | 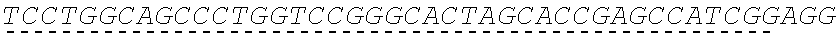 |
| | | 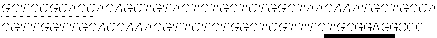 |
| | | 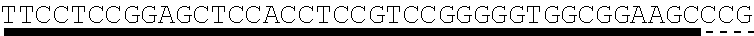 |
| | | 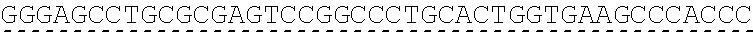 |
| | | 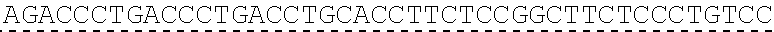 |
| | | 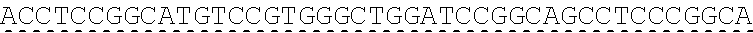 |
| | | 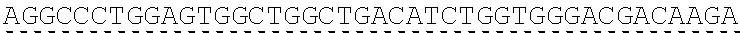 |
| | | 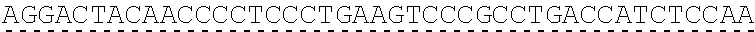 |
| | | 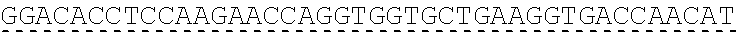 |
| | | 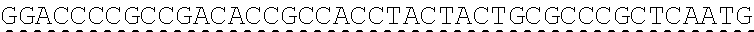 |
| | | 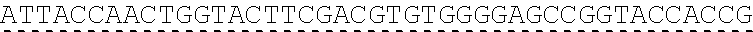 |
| | | 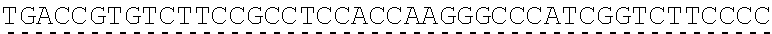 |
| | | 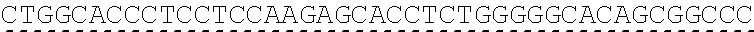 |
| | | 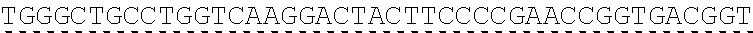 |
| | | 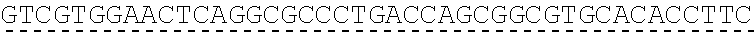 |
| | | 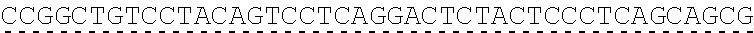 |
| | | 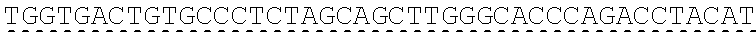 |
| | | 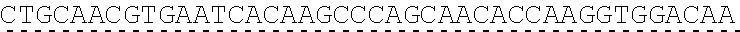 |
| | | 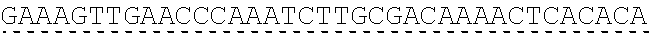 |
| Palivizumab (NH, | 173 | GACTCTTGGATGGAAGAAGTTATCAAACTGTGCGGTCGTGAACTGG |
| CEXGGGGG) Relaxin2 (30GS) | | 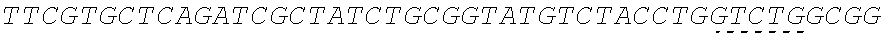 |
| | | 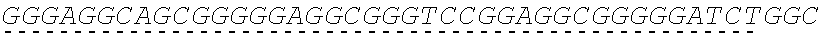 |
| | | 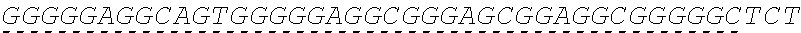 |
| | | CAGCTGTACTCTGCTCTGGCTAACAAATGCTGCCACGTTGGTTGCA |
| | | 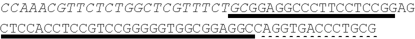 |
| | | 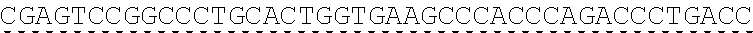 |
| | | 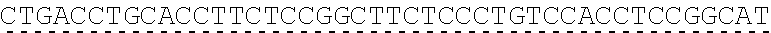 |
| | | 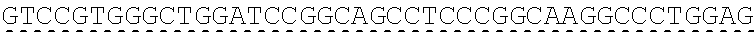 |
| | | 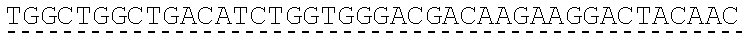 |
| | | 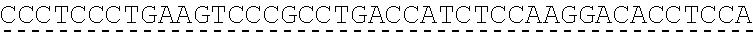 |
| | | 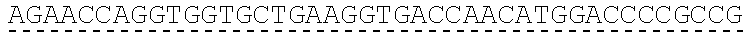 |
| | | 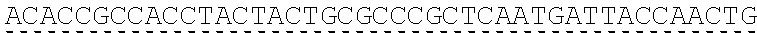 |
| | | 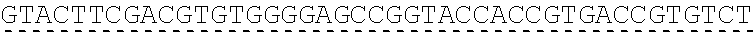 |
| | | 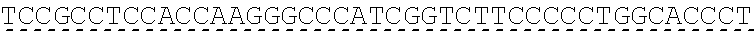 |
| | | 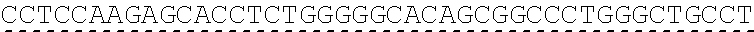 |
| | | 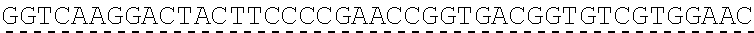 |
| | | 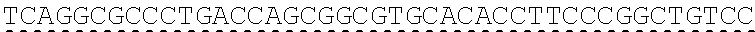 |
| | | 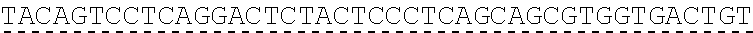 |
| | | 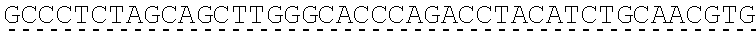 |
| | | 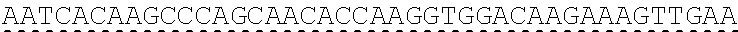 |
| | | 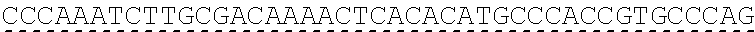 |
| | | 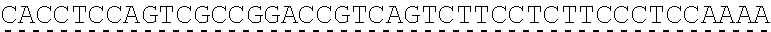 |
| | | 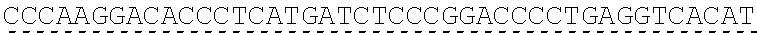 |
| | | 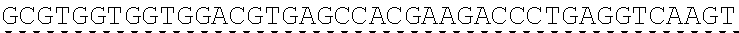 |
| | | 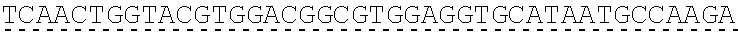 |
| | | 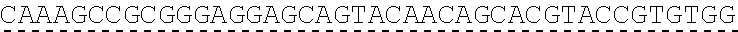 |
| | | 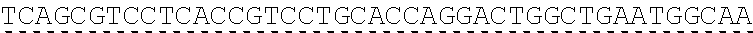 |
| | | 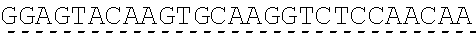 |
| | | 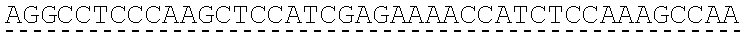 |
| | | 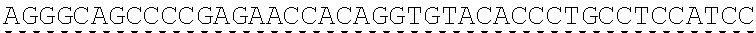 |
| | | 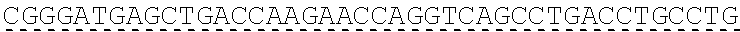 |
| | | 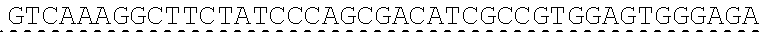 |
| | | 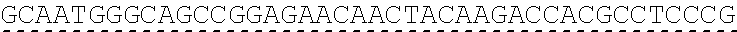 |
| | | 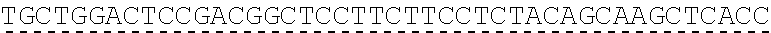 |
| | | 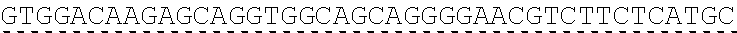 |
| | | 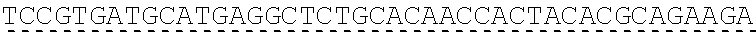 |
| | | 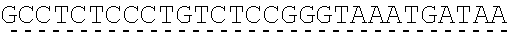 |
| Palivizumab (NH, CEXGGGGG) | 174 | GACTCTTGGATGGAAGAAGTTATCAAACTGTGCGGTCGTGAACTGG |
| Relaxin2 Q60A (30GS) | | 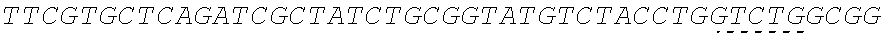 |
| | | 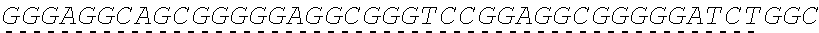 |
| | | 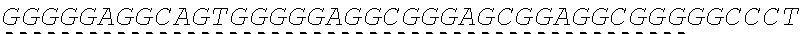 |
| | | GCGCTGTACTCTGCTCTGGCTAACAAATGCTGCCACGTTGGTTGCA |
| | | 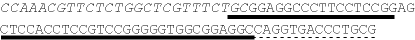 |
| | | 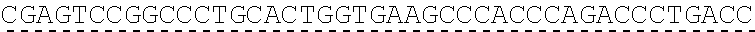 |
| | | 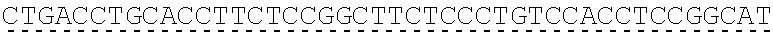 |
| | | 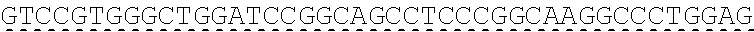 |
| | | 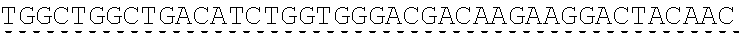 |
| | | 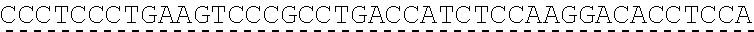 |
| | | 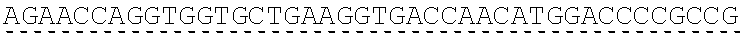 |
| | | 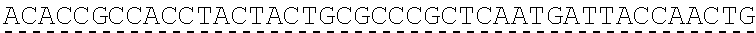 |
| | | 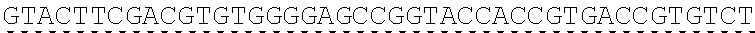 |
| | | 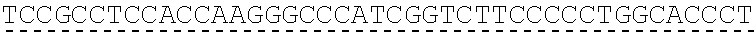 |
| | | 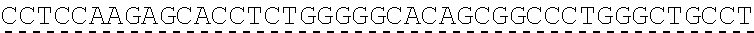 |
| | | 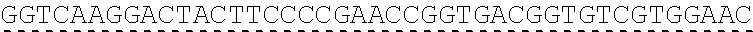 |
| | | 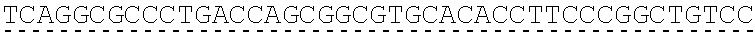 |
| | | 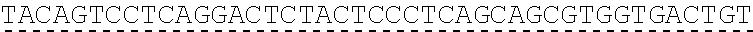 |
| | | 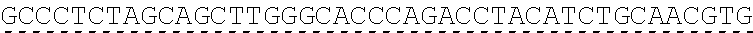 |
| | | 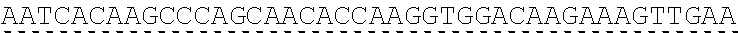 |
| | | 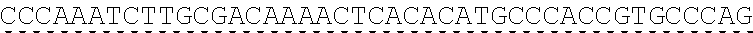 |
| | | 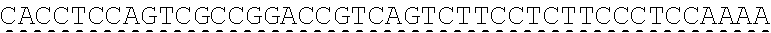 |
| | | 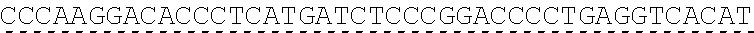 |
| | | 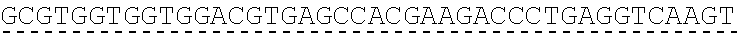 |
| | | 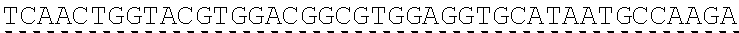 |
| | | 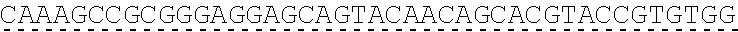 |
| | | 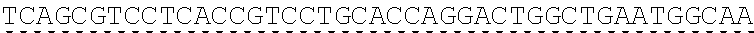 |
| | | 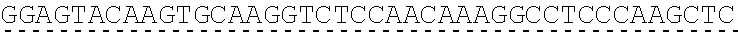 |
| | | 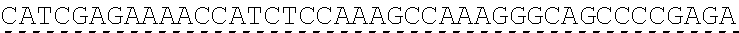 |
| | | 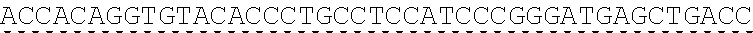 |
| | | 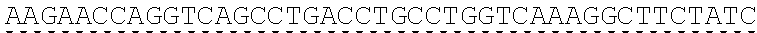 |
| | | 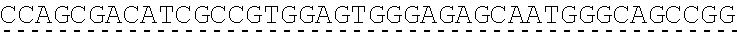 |
| | | 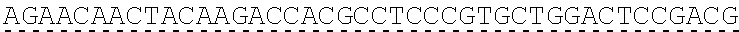 |
| | | 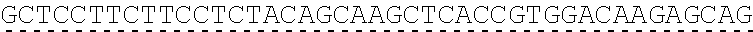 |
| | | 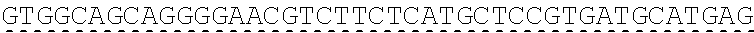 |
| | | 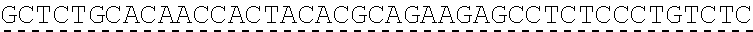 |
| | | 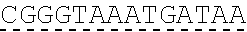 |
| Palivizumab (NH, CEXGGGGG) | 175 | GACTCTTGGATGGAAGAAGTTATCAAACTGTGCGGTCGTGAACTGG |
| Relaxin2 (9GS) | | 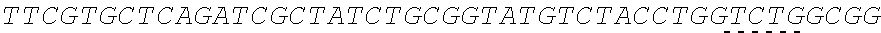 |
| | | 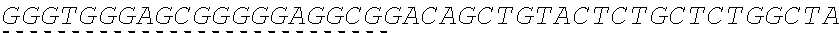 |
| | | ACAAATGCTGCCACGTTGGTTGCACCAAACGTTCTCTGGCTCGTTT |
| | | 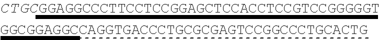 |
| | | 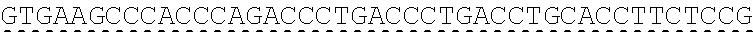 |
| | | 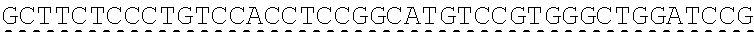 |
| | | 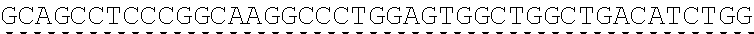 |
| | | 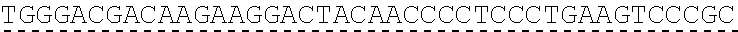 |
| | | 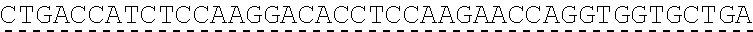 |
| | | 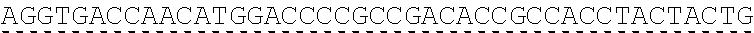 |
| | | 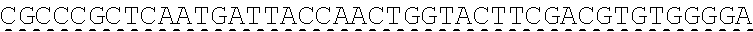 |
| | | 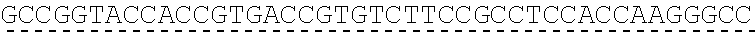 |
| | | 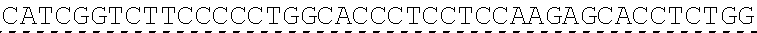 |
| | | 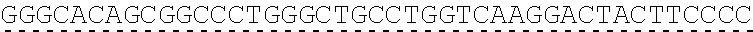 |
| | | 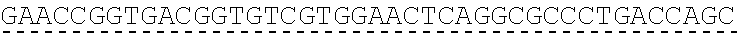 |
| | | 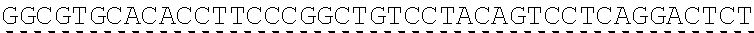 |
| | | 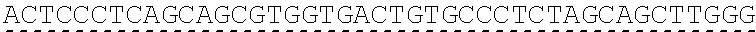 |
| | | 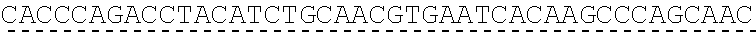 |
| | | 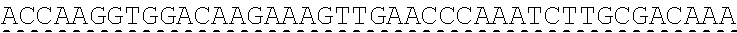 |
| | | 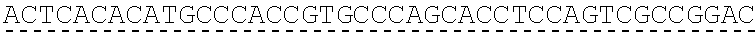 |
| | | 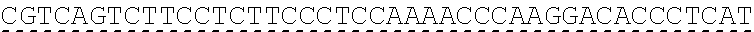 |
| | | 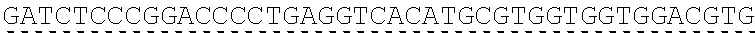 |
| | | 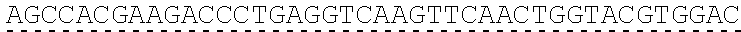 |
| | | 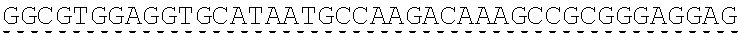 |
| | | 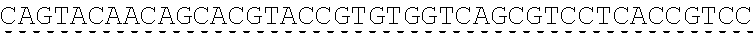 |
| | | 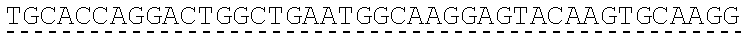 |
| | | 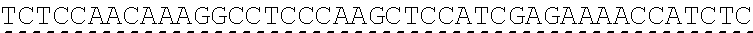 |
| | | 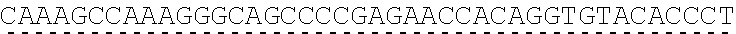 |
| | | 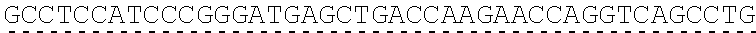 |
| | | 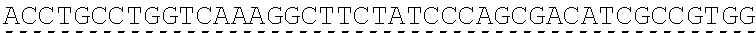 |
| | | 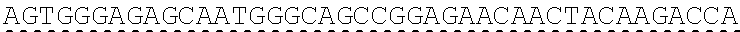 |
| | | 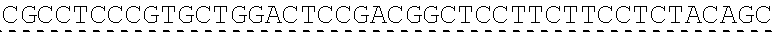 |
| | | 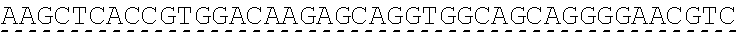 |
| | | 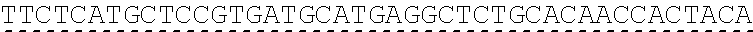 |
| | | 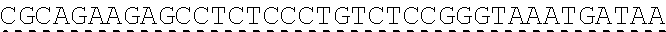 |
| Palivizumab (NH, GGGGS) | 176 | GATTCATGGATGGAGGAGGTCATCAAACTGTGTGGCAGGGAGCTG |
| Relaxin2c (9GS) | | 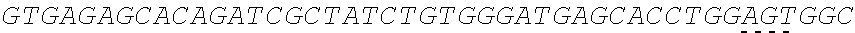 |
| | | 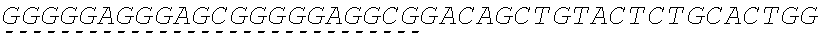 |
| | | CCAATAAATGCTGCCACGTGGGATGTACCAAGAGATCTCTGGCAC |
| | | 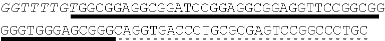 |
| | | 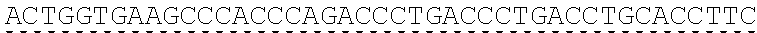 |
| | | 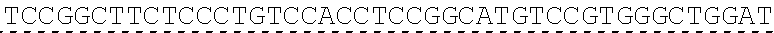 |
| | | 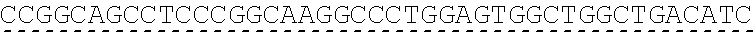 |
| | | 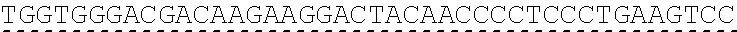 |
| | | 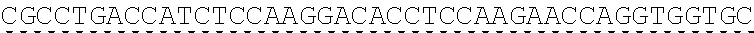 |
| | | 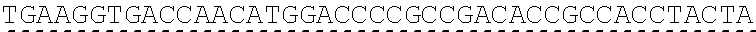 |
| | | 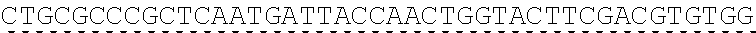 |
| | | 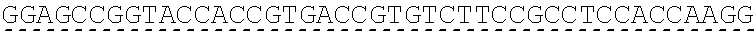 |
| | | 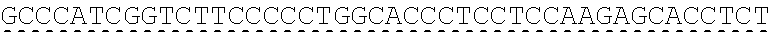 |
| | | 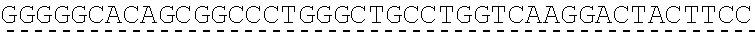 |
| | | 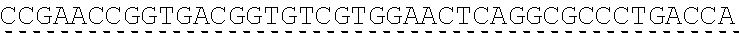 |
| | | 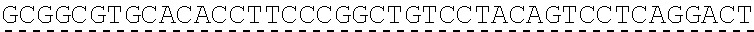 |
| | | 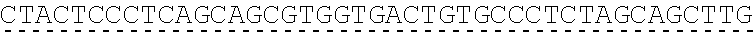 |
| | | 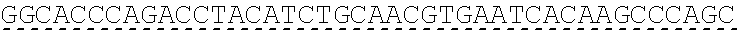 |
| | | 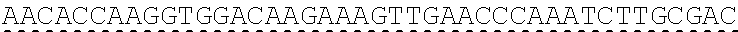 |
| | | 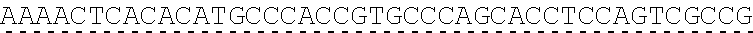 |
| | | 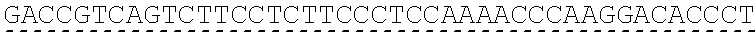 |
| | | 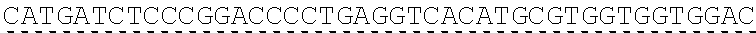 |
| | | 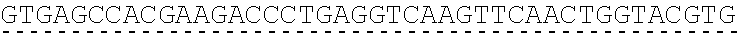 |
| | | 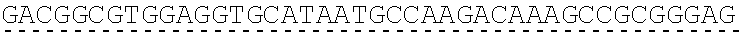 |
| | | 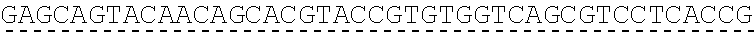 |
| | | 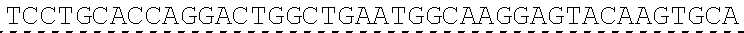 |
| | | 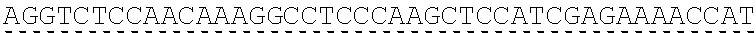 |
| | | 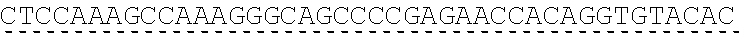 |
| | | 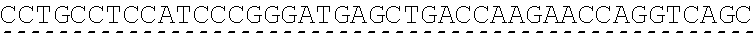 |
| | | 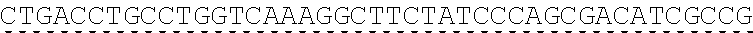 |
| | | 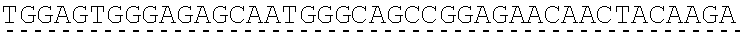 |
| | | 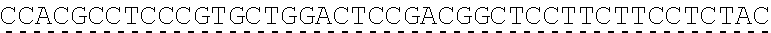 |
| | | 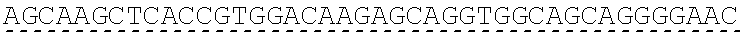 |
| | | 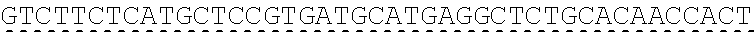 |
| | | 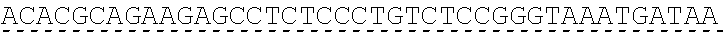 |
| Palivizumab Fab (NH, | 177 | GACTCTTGGATGGAAGAAGTTATCAAACTGTGCGGTCGTGAACTGG |
| CEXGGGGG) Relaxin2 (9GS) | | 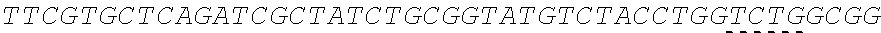 |
| | | 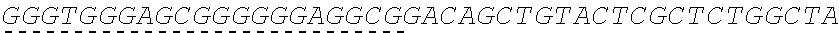 |
| | | ACAAATGCTGCCACGTTGGTTGCACCAAACGTTCTCTGGCTCGTTT |
| | | 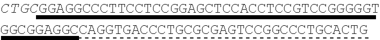 |
| | | 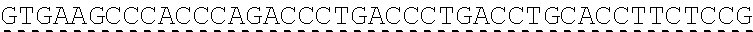 |
| | | 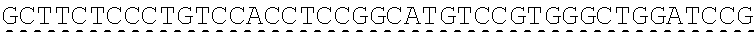 |
| | | 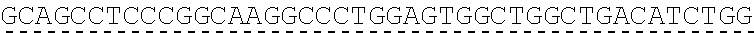 |
| | | 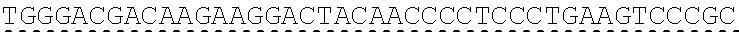 |
| | | 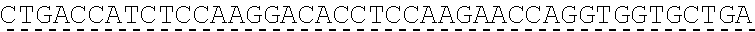 |
| | | 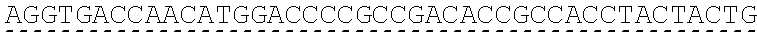 |
| | | 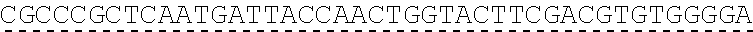 |
| | | 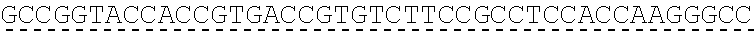 |
| | | 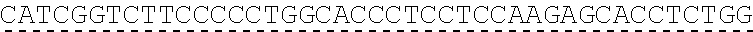 |
| | | 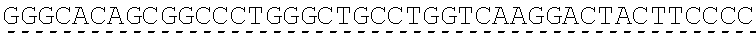 |
| | | 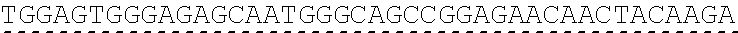 |
| | | 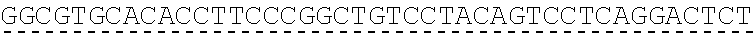 |
| | | 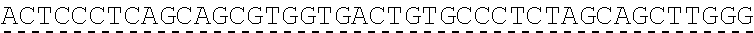 |
| | | 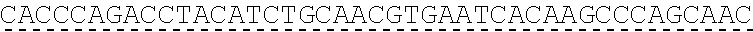 |
| | | 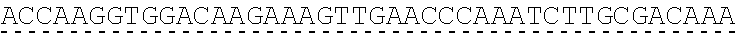 |
| | | 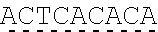 |
| Palivizumab Fab (NH, GGGGS) | 178 | GATTCATGGATGGAGGAGGTCATCAAACTGTGTGGCAGGGAGCTG |
| Relaxin2c (9GS) | | 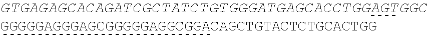 |
| | | CCAATAAATGCTGCCACGTGGGATGTACCAAGAGATCTCTGGCAC |
| | | 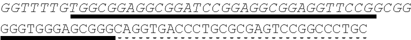 |
| | | 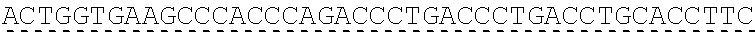 |
| | | 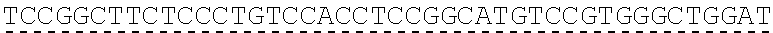 |
| | | 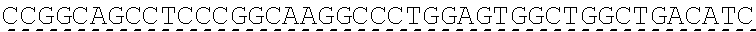 |
| | | 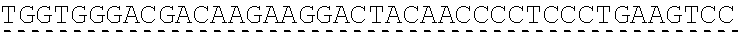 |
| | | 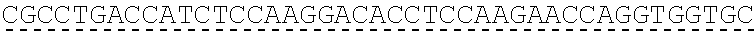 |
| | | 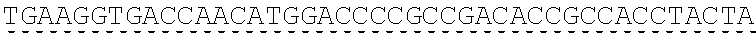 |
| | | 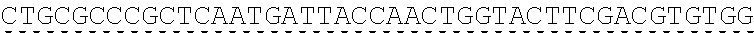 |
| | | 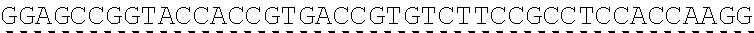 |
| | | 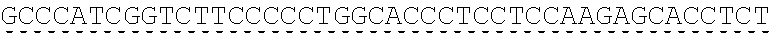 |
| | | 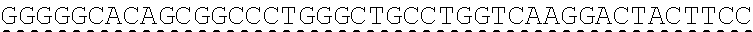 |
| | | 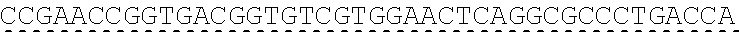 |
| | | 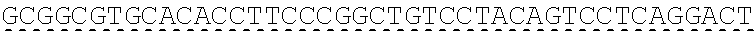 |
| | | 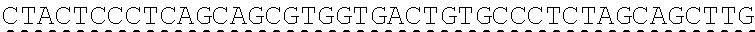 |
| | | 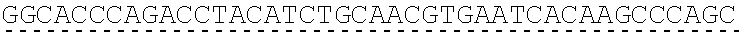 |
| | | 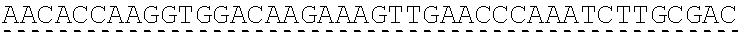 |
| | | 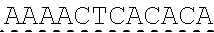 |
| Palivizumab (NH, GGGGS) | 179 | GACTCTTGGATGGAAGAAGTTATCAAACTGTGCGGTCGTGAACTGG |
| Relaxin2 (18GS) | | 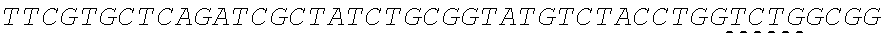 |
| | | 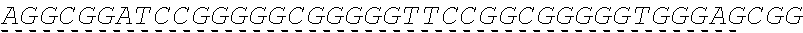 |
| | | 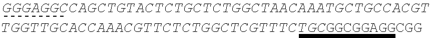 |
| | | 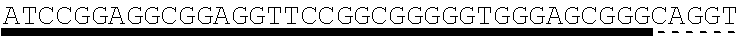 |
| | | 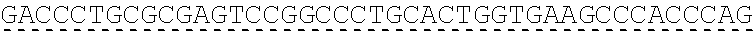 |
| | | 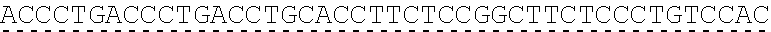 |
| | | 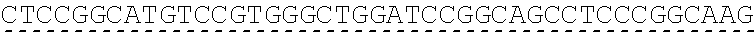 |
| | | 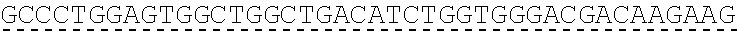 |
| | | 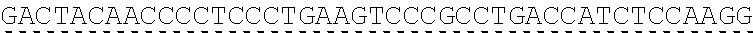 |
| | | 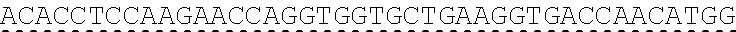 |
| | | 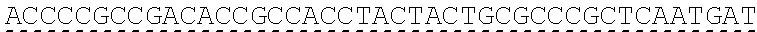 |
| | | 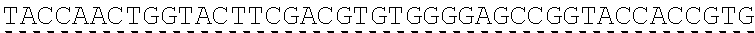 |
| | | 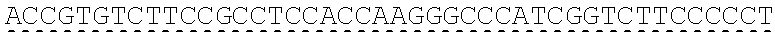 |
| | | 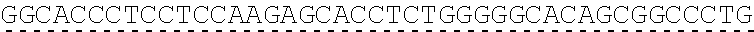 |
| | | 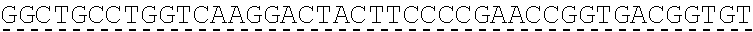 |
| | | 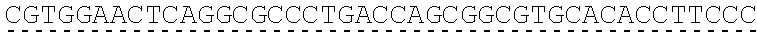 |
| | | 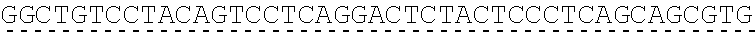 |
| | | 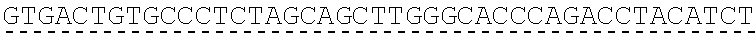 |
| | | 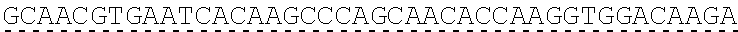 |
| | | 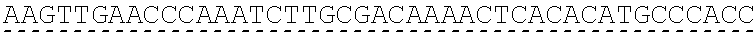 |
| | | 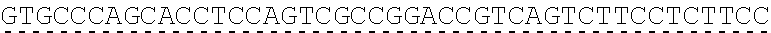 |
| | | 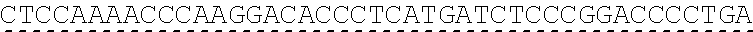 |
| | | 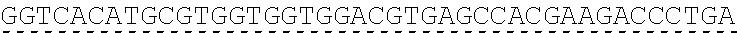 |
| | | 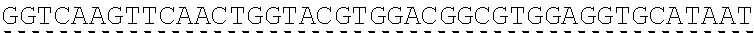 |
| | | 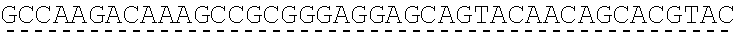 |
| | | 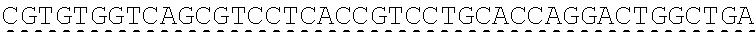 |
| | | 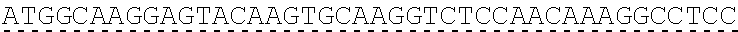 |
| | | 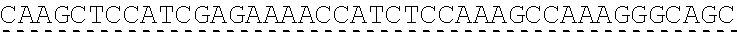 |
| | | 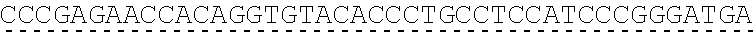 |
| | | 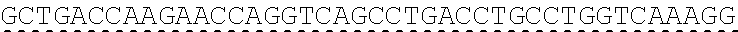 |
| | | 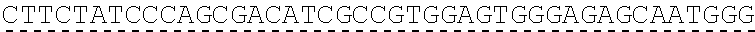 |
| | | 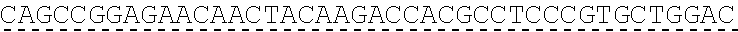 |
| | | 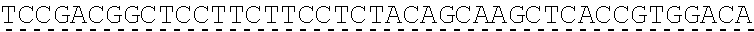 |
| | | 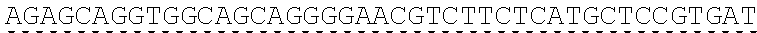 |
| | | 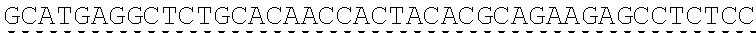 |
| | | 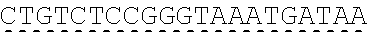 |
| Palivizumab (NH, GGGGG) | 180 | GACTCTTGGATGGAAGAAGTTATCAAACTGTGCGGTCGTGAACTGG |
| Relaxin2 (GGGPRR) | | 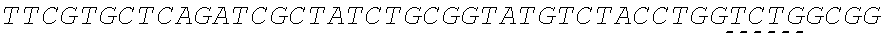 |
| | | 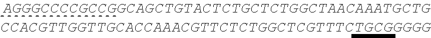 |
| | | 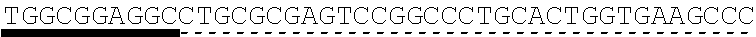 |
| | | 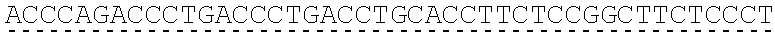 |
| | | 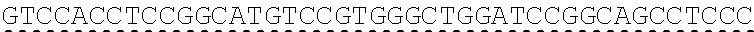 |
| | | 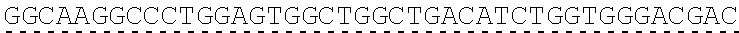 |
| | | 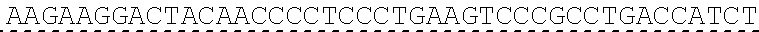 |
| | | 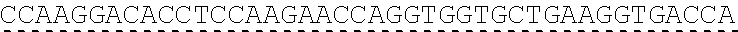 |
| | | 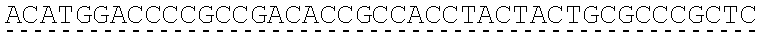 |
| | | 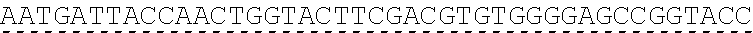 |
| | | 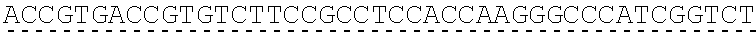 |
| | | 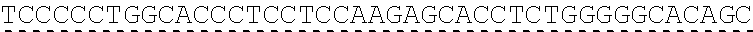 |
| | | 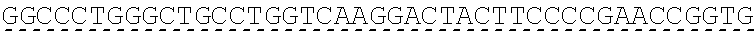 |
| | | 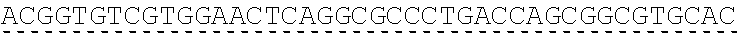 |
| | | 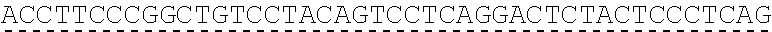 |
| | | 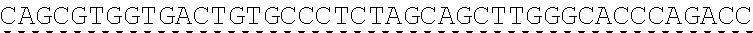 |
| | | 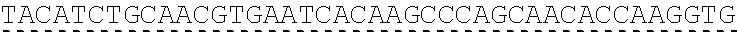 |
| | | 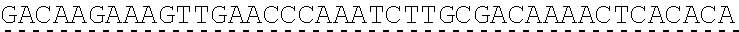 |
| | | 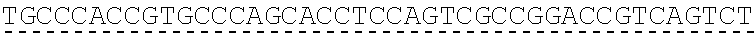 |
| | | 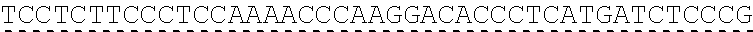 |
| | | 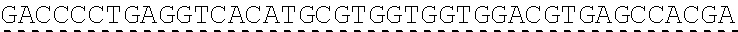 |
| | | 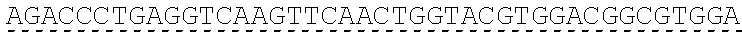 |
| | | 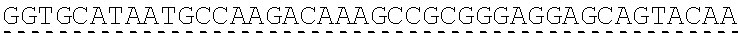 |
| | | 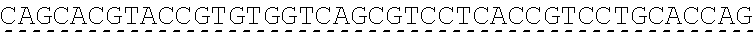 |
| | | 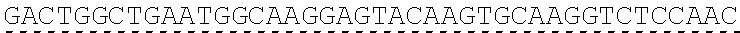 |
| | | 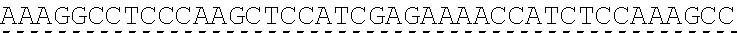 |
| | | 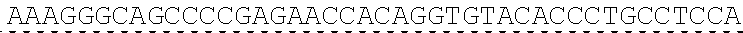 |
| | | 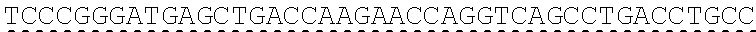 |
| | | 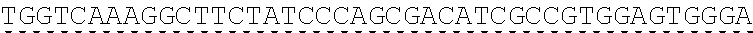 |
| | | 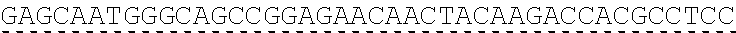 |
| | | 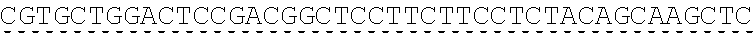 |
| | | 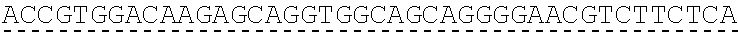 |
| | | 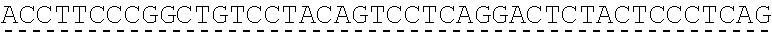 |
| | | 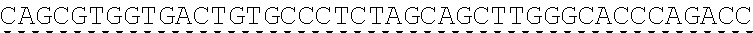 |
| Palivizumab (NH,CEXGGGGG) | 181 | GACTCTTGGATGGAAGAAGTTATCAAACTGTGCGGTCGTGAACTGG |
| Relaxin2 (GGGPRR) | | 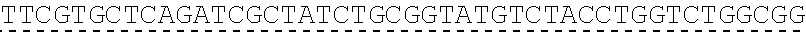 |
| | | 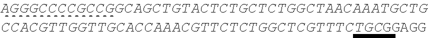 |
| | | 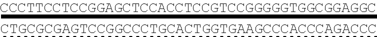 |
| | | 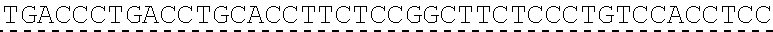 |
| | | 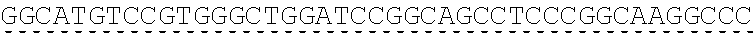 |
| | | 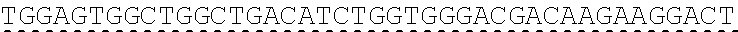 |
| | | 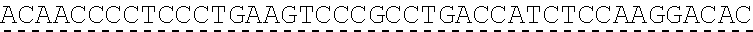 |
| | | 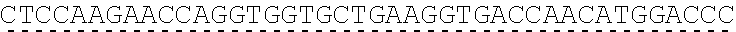 |
| | | 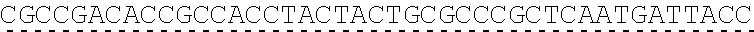 |
| | | 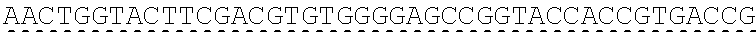 |
| | | 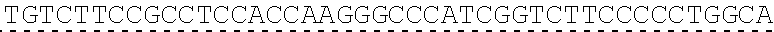 |
| | | 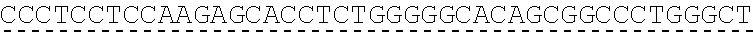 |
| | | 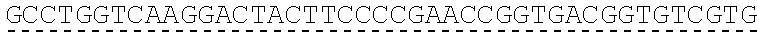 |
| | | 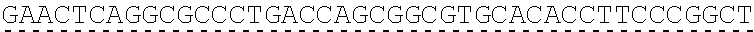 |
| | | 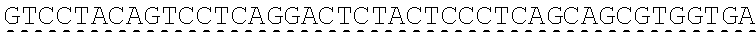 |
| | | 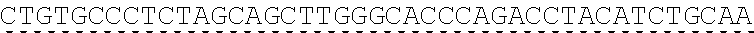 |
| | | 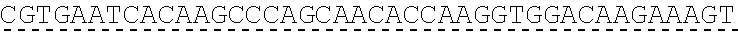 |
| | | 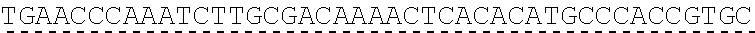 |
| | | 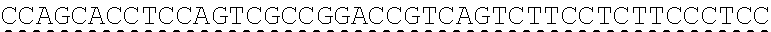 |
| | | 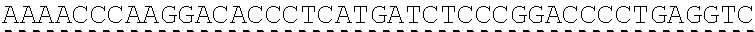 |
| | | 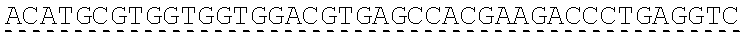 |
| | | 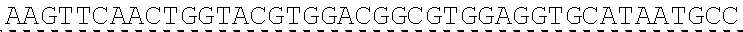 |
| | | 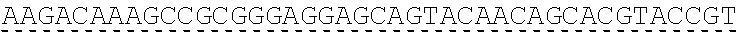 |
| | | 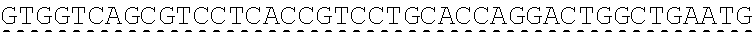 |
| | | 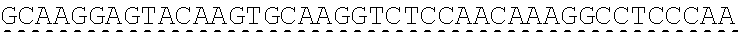 |
| | | 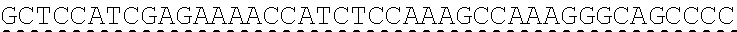 |
| | | 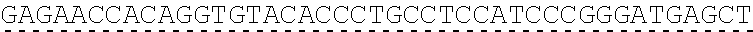 |
| | | 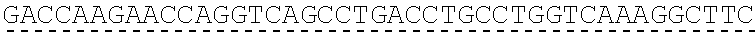 |
| | | 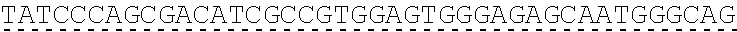 |
| | | 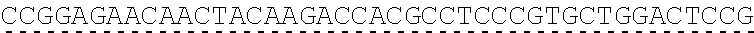 |
| | | 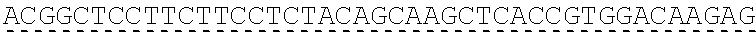 |
| | | 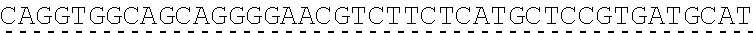 |
| | | 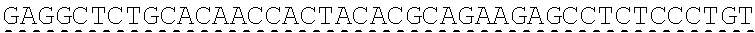 |
| | | 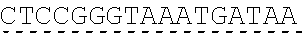 |
| Palivizumab (NH, EAAAK) | 182 | GACTCTTGGATGGAAGAAGTTATCAAACTGTGCGGTCGTGAACTGG |
| Relaxin2 (GGGPRR) | | 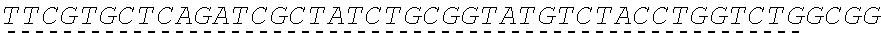 |
| | | 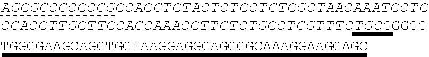 |
| | | 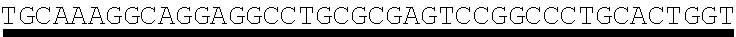 |
| | | 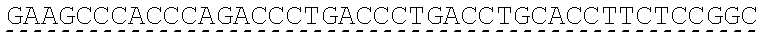 |
| | | 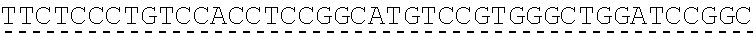 |
| | | 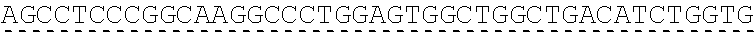 |
| | | 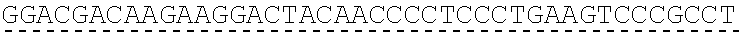 |
| | | 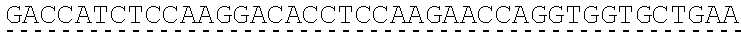 |
| | | 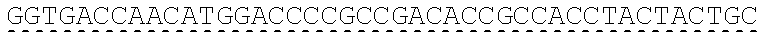 |
| | | 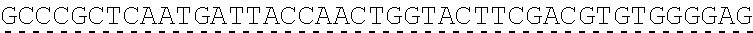 |
| | | 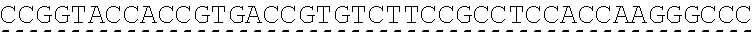 |
| | | 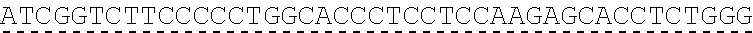 |
| | | 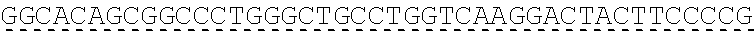 |
| | | 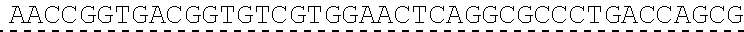 |
| | | 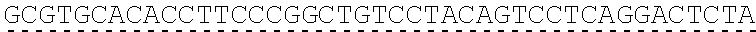 |
| | | 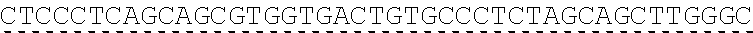 |
| | | 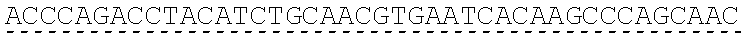 |
| | | 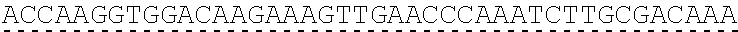 |
| | | 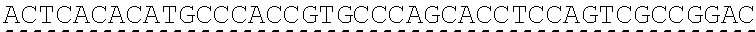 |
| | | 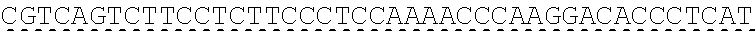 |
| | | 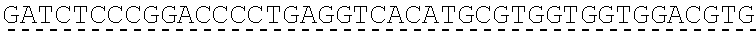 |
| | | 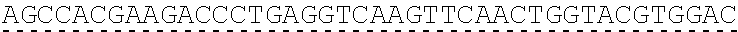 |
| | | 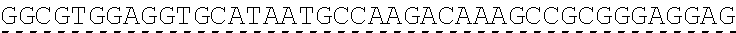 |
| | | 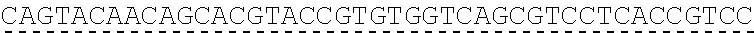 |
| | | 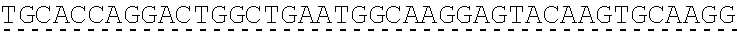 |
| | | 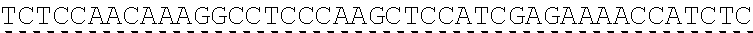 |
| | | 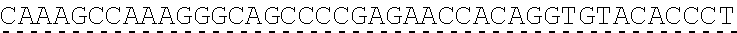 |
| | | 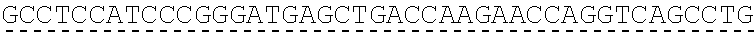 |
| | | 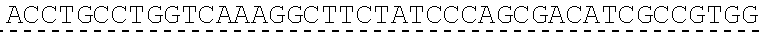 |
| | | 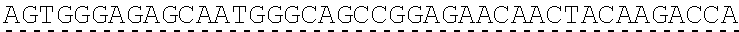 |
| | | 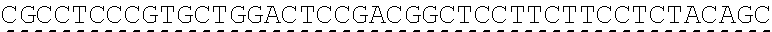 |
| | | 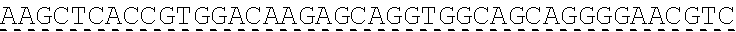 |
| | | 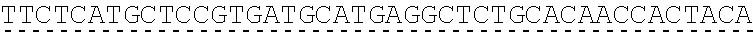 |
| | | 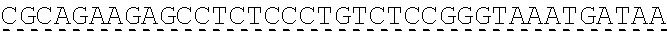 |
| Palivizumab (NL, CEXGGGGG) | 183 | GACTCTTGGATGGAAGAAGTTATCAAACTGTGCGGTCGTGAACTGG |
| Relaxin2 (GGGPRR) | | 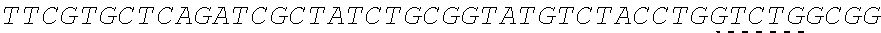 |
| | | 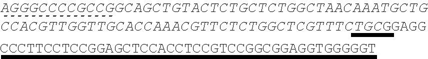 |
| | | 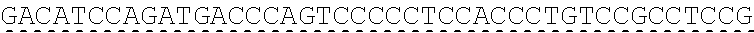 |
| | | 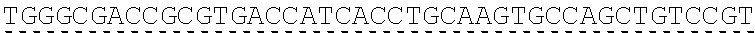 |
| | | 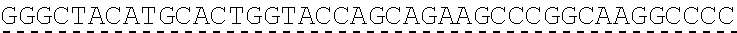 |
| | | 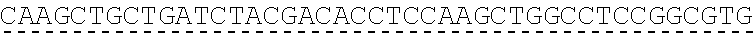 |
| | | 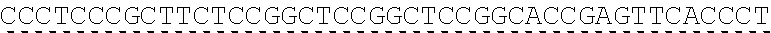 |
| | | 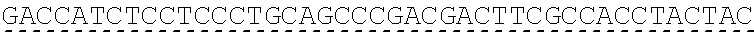 |
| | | 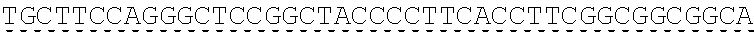 |
| | | 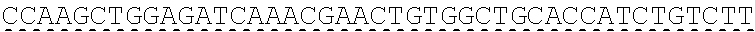 |
| | | 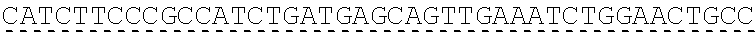 |
| | | 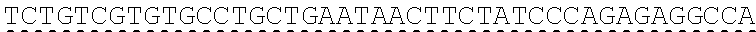 |
| | | 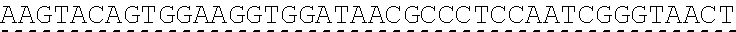 |
| | | 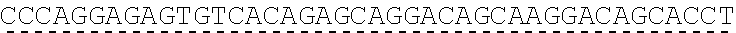 |
| | | 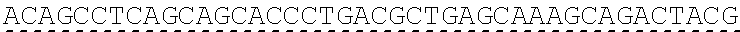 |
| | | 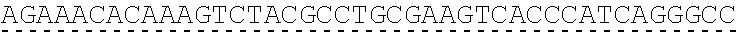 |
| | | 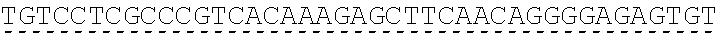 |
| Palivizumab (NL, EAAAK) | 184 | GACTCTTGGATGGAAGAAGTTATCAAACTGTGCGGTCGTGAACTGG |
| Relaxin2 (GGGPRR) | | 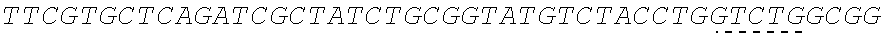 |
| | | 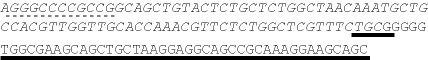 |
| | | 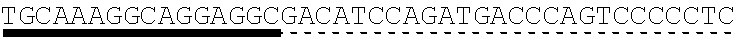 |
| | | 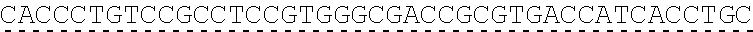 |
| | | 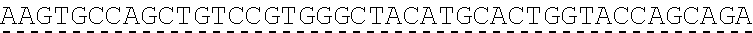 |
| | | 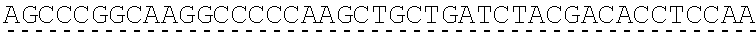 |
| | | 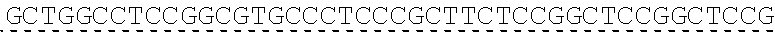 |
| | | 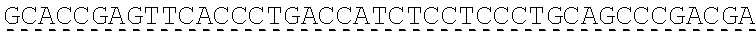 |
| | | 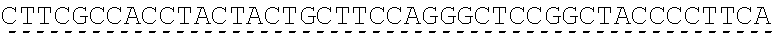 |
| | | 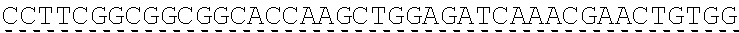 |
| | | 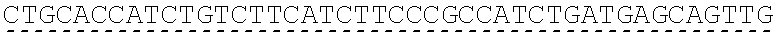 |
| | | 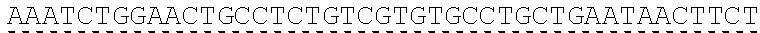 |
| | | 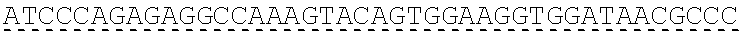 |
| | | 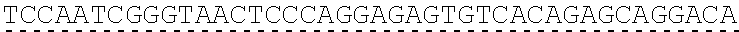 |
| | | 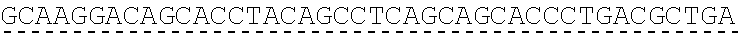 |
| | | 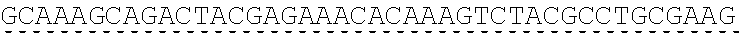 |
| | | 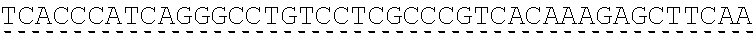 |
| | | 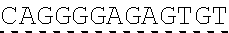 |
| Palivizumab(NH, CEXGGGGS) | 185 | CATGGTGAAGGGACCTTTACCAGTGATGTAAGTTCTTATTTGGAAG |
| GLP1 | | 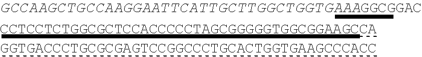 |
| | | 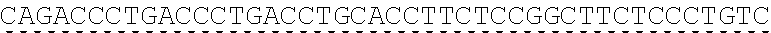 |
| | | 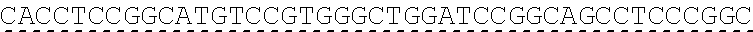 |
| | | 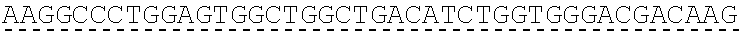 |
| | | 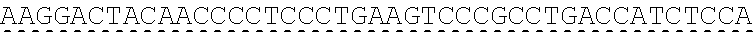 |
| | | 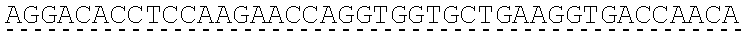 |
| | | 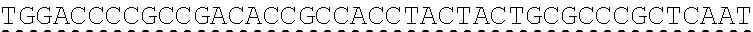 |
| | | 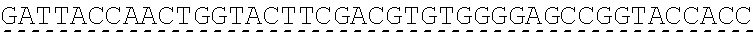 |
| | | 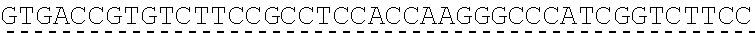 |
| | | 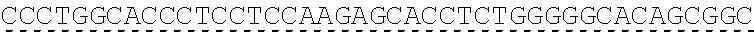 |
| | | 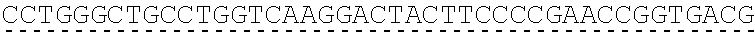 |
| | | 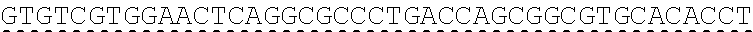 |
| | | 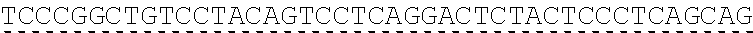 |
| | | 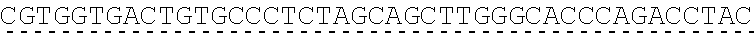 |
| | | 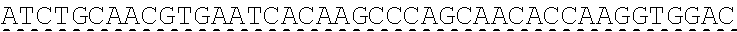 |
| | | 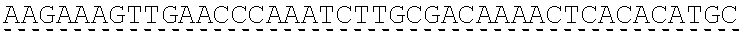 |
| | | 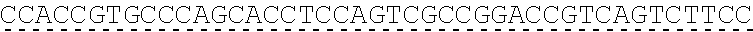 |
| | | 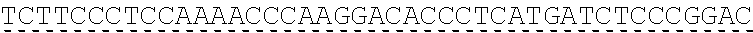 |
| | | 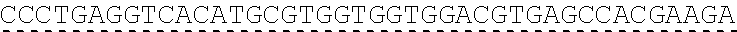 |
| | | 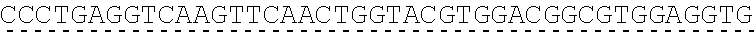 |
| | | 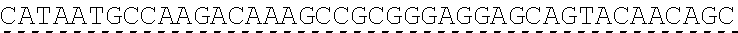 |
| | | 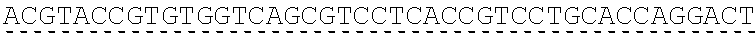 |
| | | 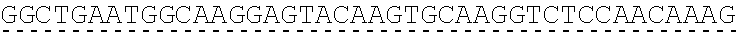 |
| | | 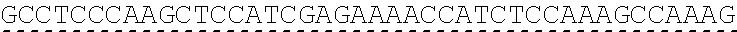 |
| | | 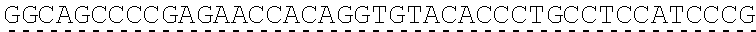 |
| | | 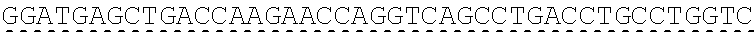 |
| | | 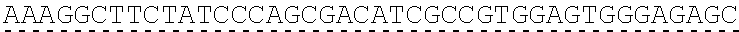 |
| | | 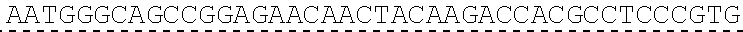 |
| | | 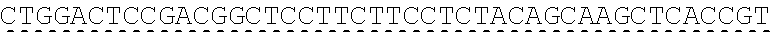 |
| | | 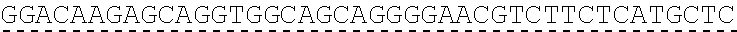 |
| | | 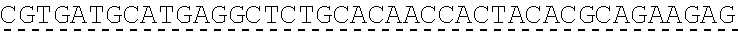 |
| | | 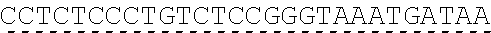 |
| Palivizumab (NH, GGGGS) | 186 | CATGGTGAAGGGACCTTTACCAGTGATGTAAGTTCTTATTTGGAAG |
| GLP1 | | 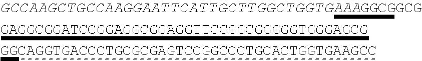 |
| | | 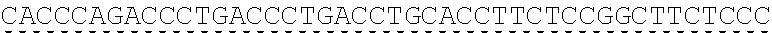 |
| | | 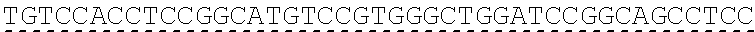 |
| | | 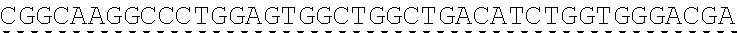 |
| | | 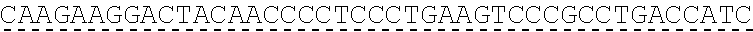 |
| | | 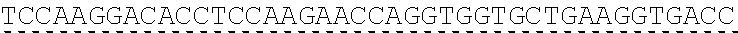 |
| | | 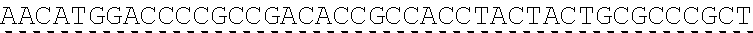 |
| | | 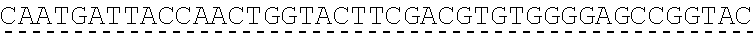 |
| | | 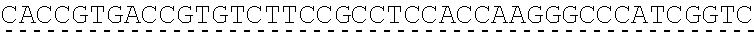 |
| | | 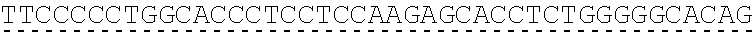 |
| | | 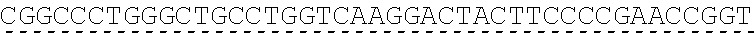 |
| | | 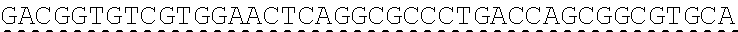 |
| | | 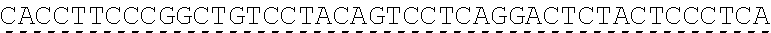 |
| | | 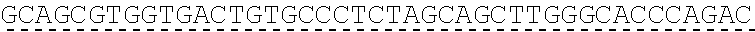 |
| | | 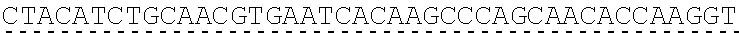 |
| | | 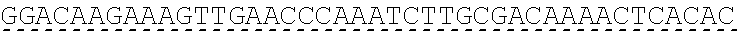 |
| | | 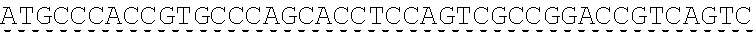 |
| | | 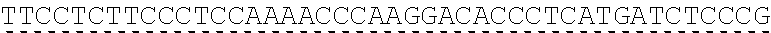 |
| | | 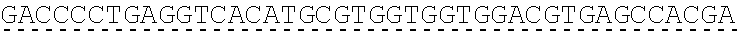 |
| | | 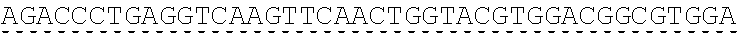 |
| | | 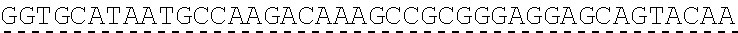 |
| | | 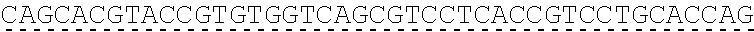 |
| | | 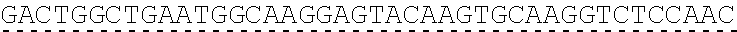 |
| | | 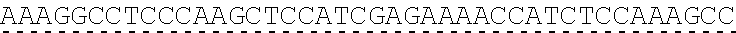 |
| | | 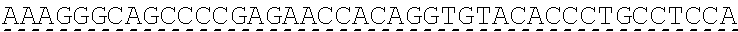 |
| | | 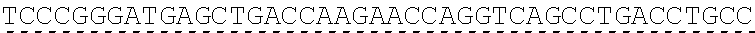 |
| | | 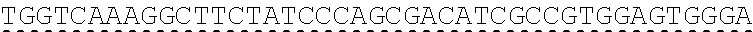 |
| | | 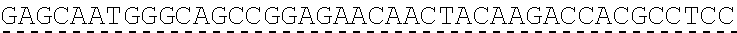 |
| | | 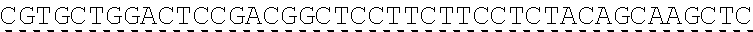 |
| | | 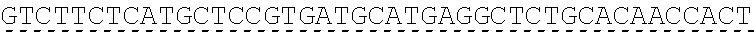 |
| | | 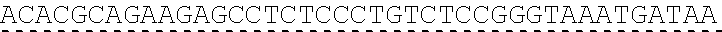 |
| | | 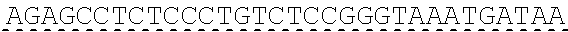 |
| Palivizumab (NH, CEXGGGGS) | 187 | CACGGCGACGGTTCATTCTCTGACGAAATGAATACAATACTCGACA |
| GLP2 | | ACCTCGCCGCCAGGGACTTTATCAATTGGCTCATTCAAACTAAAAT |
| | | 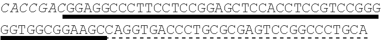 |
| | | 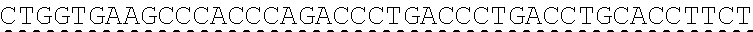 |
| | | 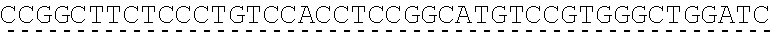 |
| | | 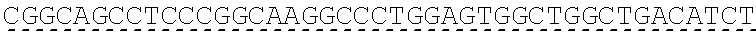 |
| | | 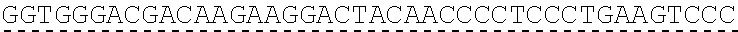 |
| | | 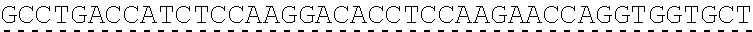 |
| | | 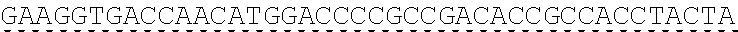 |
| | | 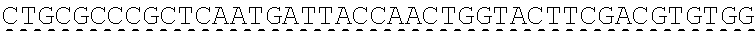 |
| | | 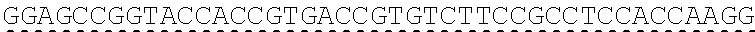 |
| | | 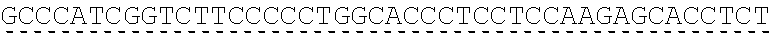 |
| | | 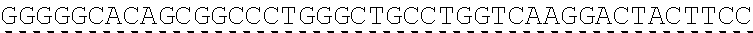 |
| | | 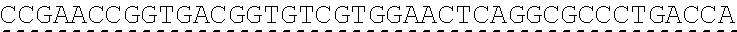 |
| | | 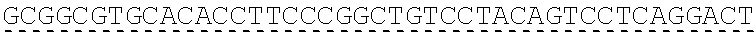 |
| | | 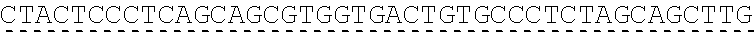 |
| | | 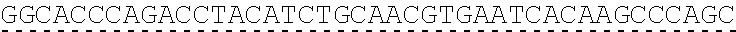 |
| | | 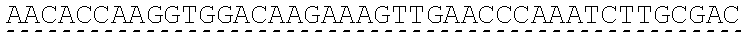 |
| | | 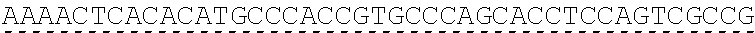 |
| | | 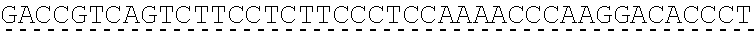 |
| | | 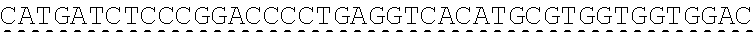 |
| | | 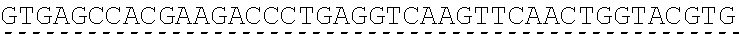 |
| | | 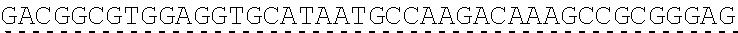 |
| | | 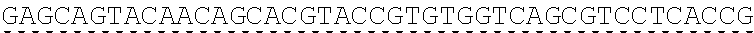 |
| | | 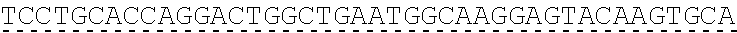 |
| | | 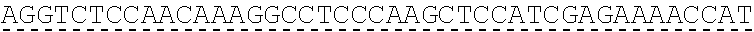 |
| | | 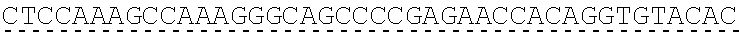 |
| | | 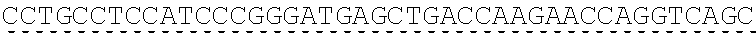 |
| | | 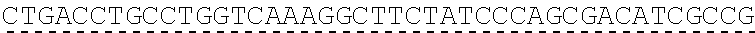 |
| | | 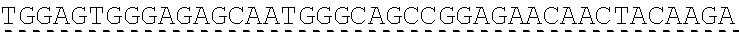 |
| | | 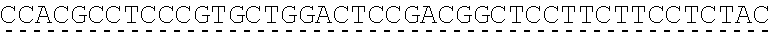 |
| | | 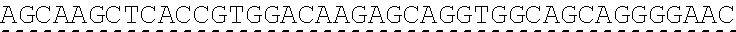 |
| | | 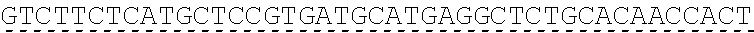 |
| | | 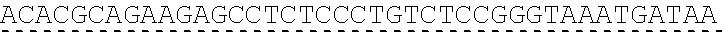 |
| Palivizumab (NH, GGGGG) GLP2 | 188 | CACGGCGACGGTTCATTCTCTGACGAAATGAATACAATACTCGACA |
| | | ACCTCGCCGCCAGGGACTTTATCAATTGGCTCATTCAAACTAAAAT |
| | | 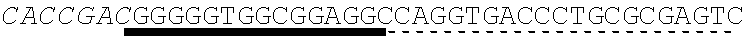 |
| | | 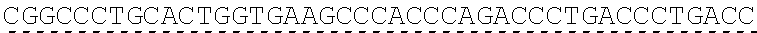 |
| | | 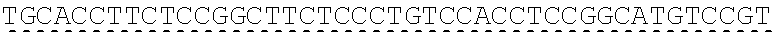 |
| | | 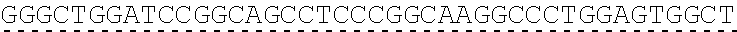 |
| | | 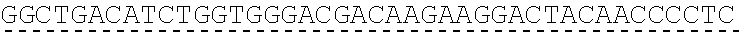 |
| | | 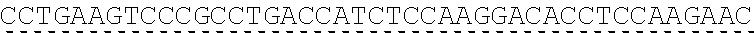 |
| | | 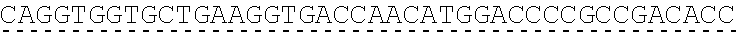 |
| | | 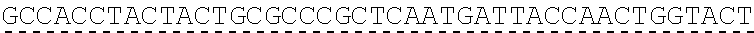 |
| | | 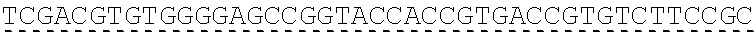 |
| | | 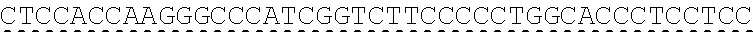 |
| | | 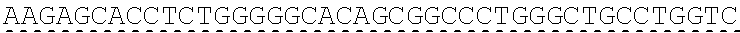 |
| | | 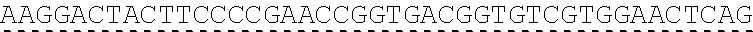 |
| | | 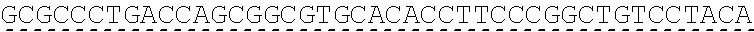 |
| | | 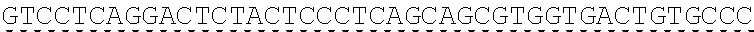 |
| | | 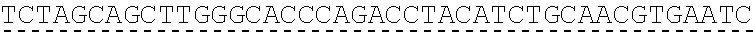 |
| | | 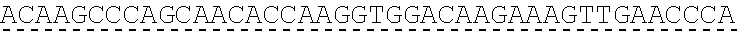 |
| | | 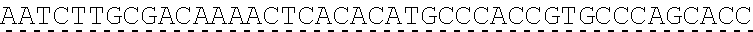 |
| | | 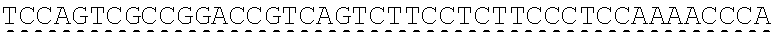 |
| | | 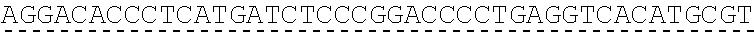 |
| | | 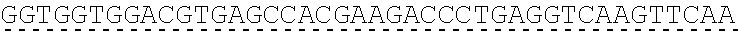 |
| | | 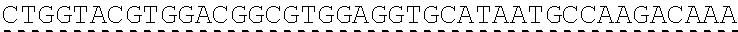 |
| | | 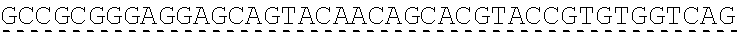 |
| | | 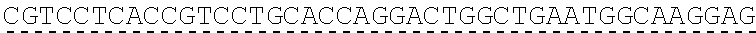 |
| | | 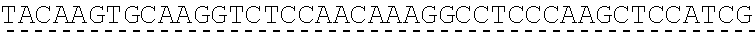 |
| | | 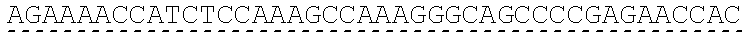 |
| | | 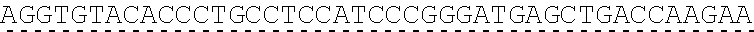 |
| | | 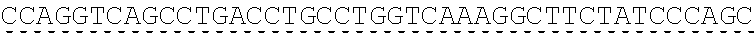 |
| | | 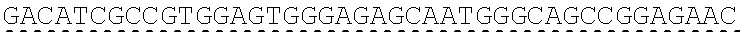 |
| | | 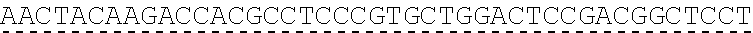 |
| | | 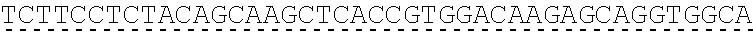 |
| | | 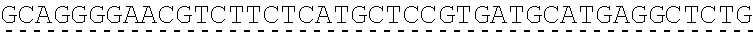 |
| | | 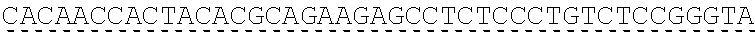 |
| | | 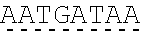 |
| Palivizumab (NH, EAAAK) GLP2 | 189 | CACGGCGACGGTTCATTCTCTGACGAAATGAATACAATACTCGACA |
| | | ACCTCGCCGCCAGGGACTTTATCAATTGGCTCATTCAAACTAAAAT |
| | | 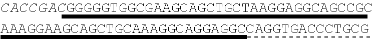 |
| | | 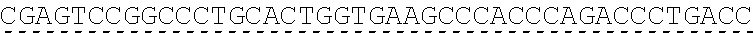 |
| | | 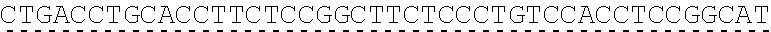 |
| | | 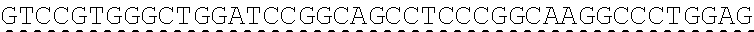 |
| | | 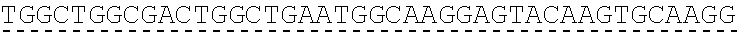 |
| | | 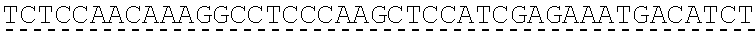 |
| | | 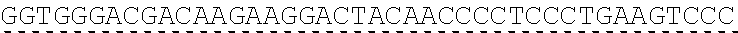 |
| | | 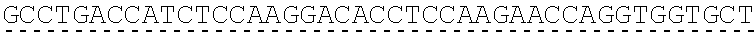 |
| | | 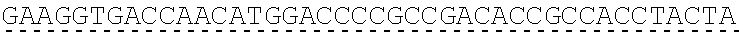 |
| | | 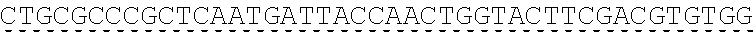 |
| | | 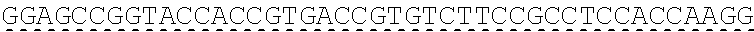 |
| | | 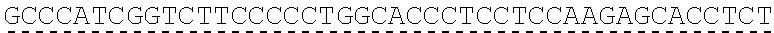 |
| | | 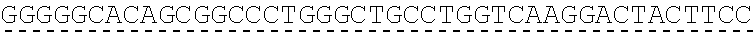 |
| | | 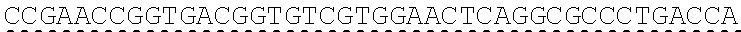 |
| | | 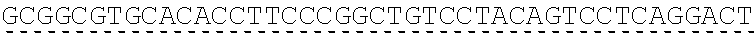 |
| | | 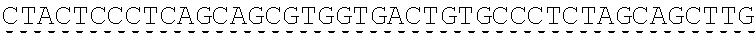 |
| | | 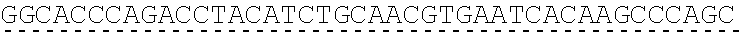 |
| | | 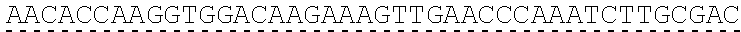 |
| | | 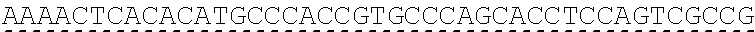 |
| | | 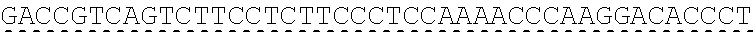 |
| | | 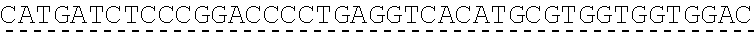 |
| | | 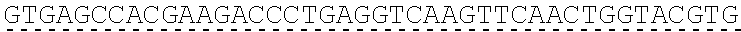 |
| | | 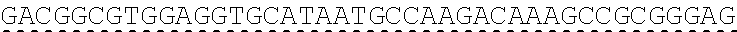 |
| | | 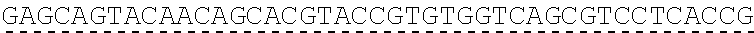 |
| | | 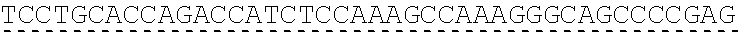 |
| | | 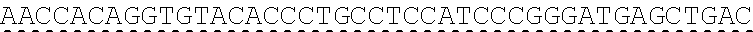 |
| | | 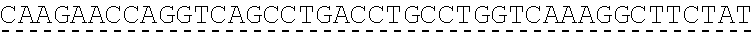 |
| | | 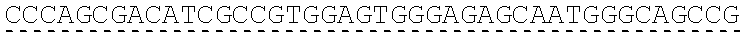 |
| | | 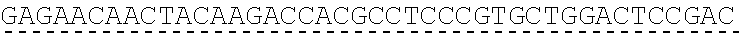 |
| | | 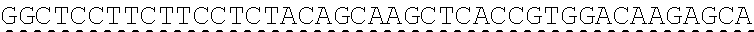 |
| | | 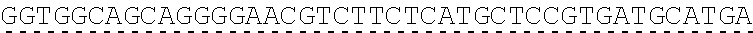 |
| | | 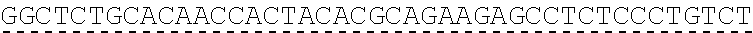 |
| | | 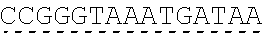 |
| Palivizumab (NL, EAAAK) | 190 | CACGGCGACGGTTCATTCTCTGACGAAATGAATACAATACTCGACA |
| GLP2 | | ACCTCGCCGCCAGGGACTTTATCAATTGGCTCATTCAAACTAAAAT |
| | | 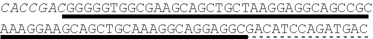 |
| | | 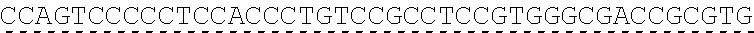 |
| | | 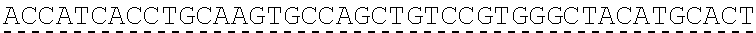 |
| | | 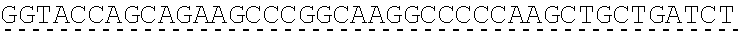 |
| | | 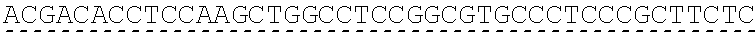 |
| | | 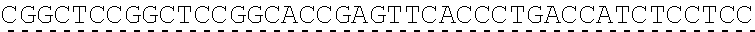 |
| | | 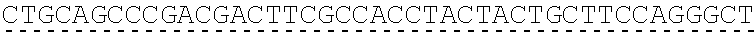 |
| | | 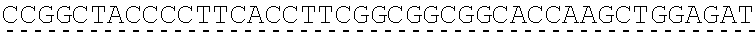 |
| | | 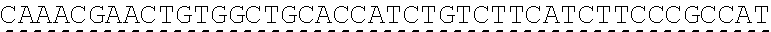 |
| | | 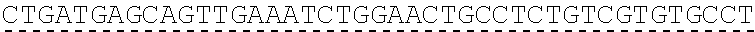 |
| | | 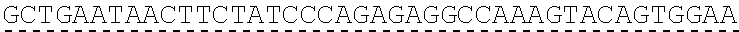 |
| | | 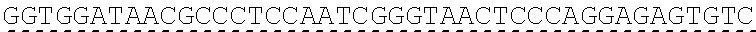 |
| | | 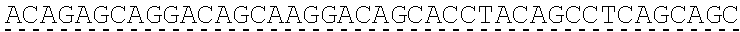 |
| | | 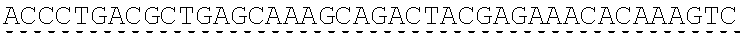 |
| | | 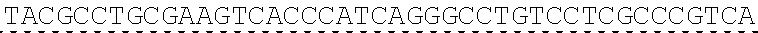 |
| | | 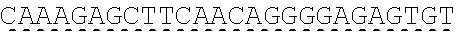 |
| Palivizumab (NH, EAAAK) | 191 | GACTCTTGGATGGAAGAAGTTATCAAACTGTGCGGTCGTGAACTGG |
| Relaxin (dual) | | TTCGTGCTCAGATCGCTATCTGCGGTATGTCTACCTGGTCTAAACG |
| | | 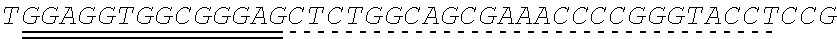 |
| | | 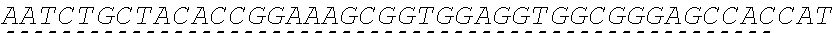 |
| | | 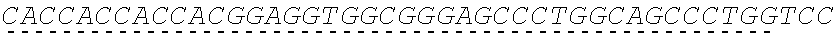 |
| | | 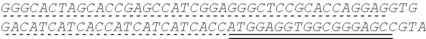 |
| | | AAAAACGTCAGCTGTACTCTGCTCTGGCTAACAAATGCTGCCACGT |
| | | 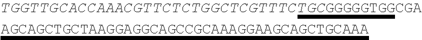 |
| | | 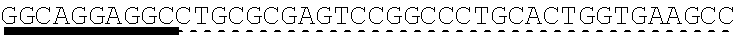 |
| | | 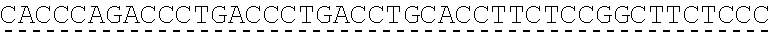 |
| | | 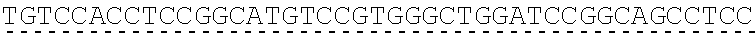 |
| | | 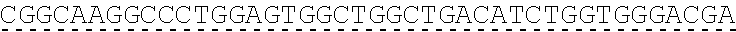 |
| | | 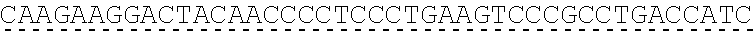 |
| | | 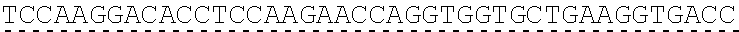 |
| | | 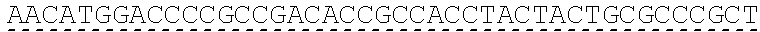 |
| | | 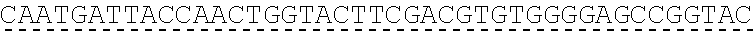 |
| | | 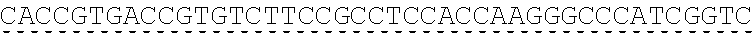 |
| | | 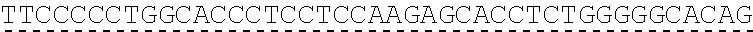 |
| | | 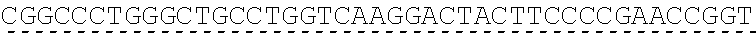 |
| | | 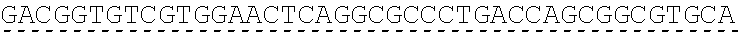 |
| | | 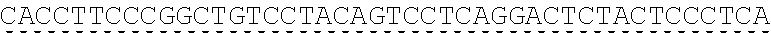 |
| | | 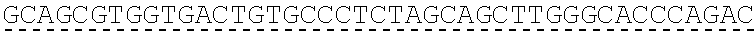 |
| | | 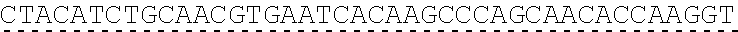 |
| | | 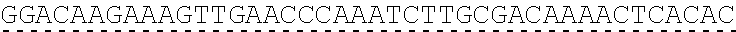 |
| | | 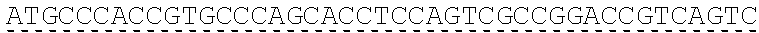 |
| | | 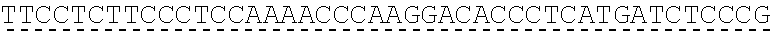 |
| | | 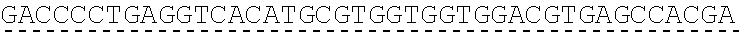 |
| | | 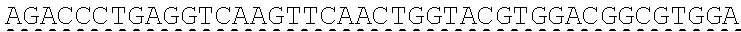 |
| | | 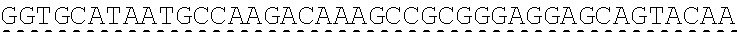 |
| | | 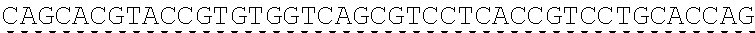 |
| | | 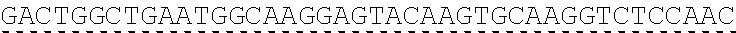 |
| | | 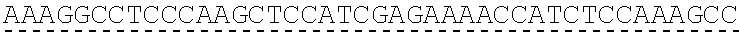 |
| | | 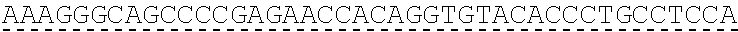 |
| | | 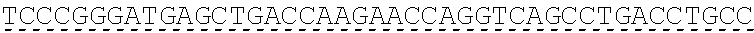 |
| | | 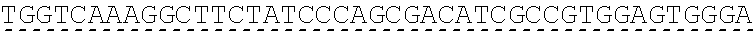 |
| | | 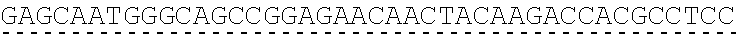 |
| | | 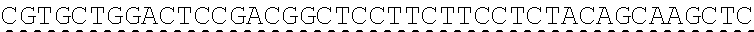 |
| | | 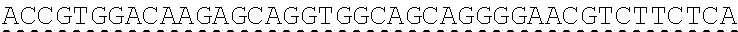 |
| | | 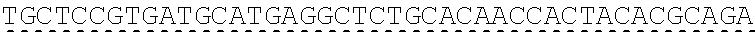 |
| | | 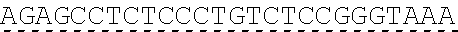 |
| Palivizumab (NH, EAAAK) | 265 | GACTCTTGGATGGAAGAAGTTATCAAACTGTGCGGTCGTGAACTGG |
| Relaxin2 (single) | | 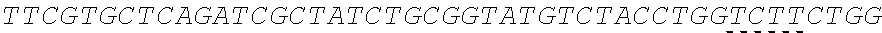 |
| | | 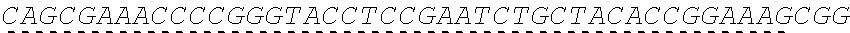 |
| | | 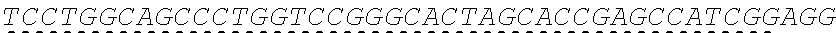 |
| | | 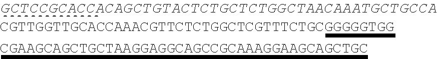 |
| | | 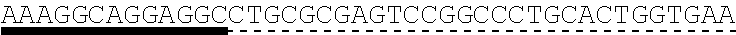 |
| | | 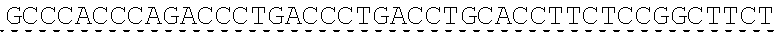 |
| | | 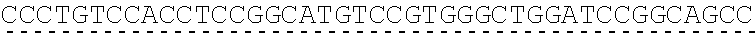 |
| | | 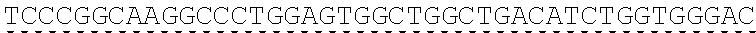 |
| | | 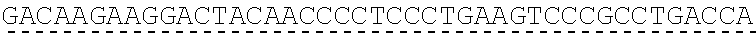 |
| | | 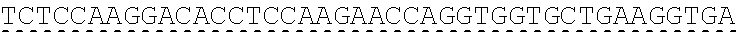 |
| | | 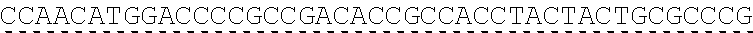 |
| | | 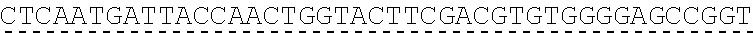 |
| | | 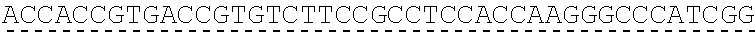 |
| | | 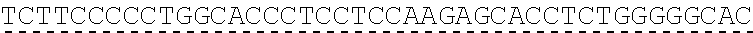 |
| | | 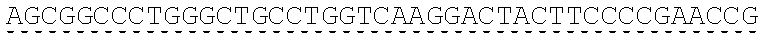 |
| | | 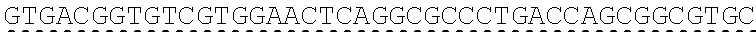 |
| | | 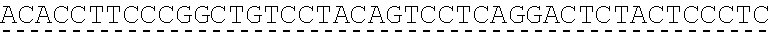 |
| | | 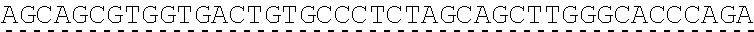 |
| | | 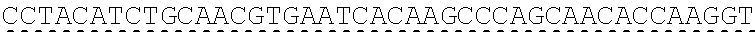 |
| | | 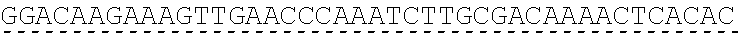 |
| | | 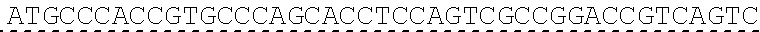 |
| | | 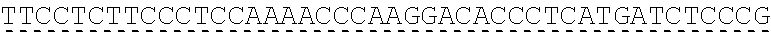 |
| | | 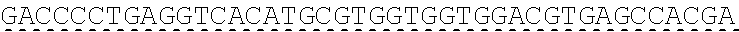 |
| | | 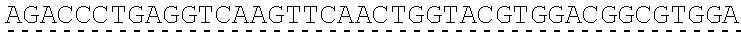 |
| | | 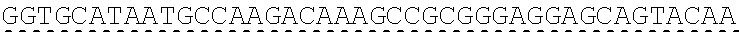 |
| | | 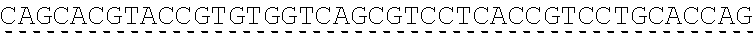 |
| | | 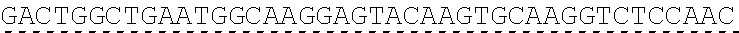 |
| | | 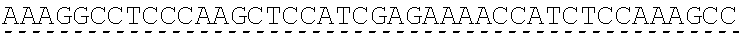 |
| | | 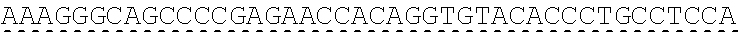 |
| | | 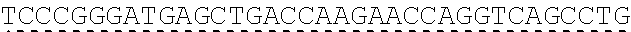 |

For SEQ ID NOs: 9-41, 161-190, 161-191, 265 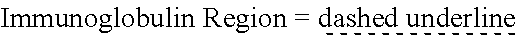
Peptide/Therapeutic peptide = italic 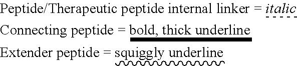
Linker = double underline
Protease site: underline

TABLE 4
Immunoglobulin fusion protein - Amino Acid Sequence

| Name | SEQ ID NO | Sequence |
|---|---|---|
| Trastuzumab | 42 | 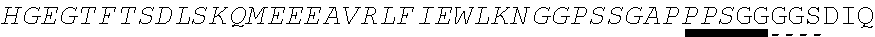 |
| (NL, GGGGS) | | 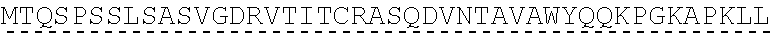 |
| Exendin-4 | | 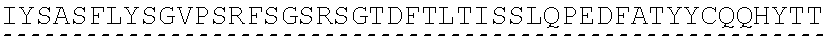 |
| | | 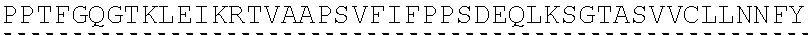 |
| | | 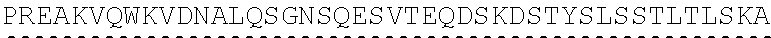 |
| | | 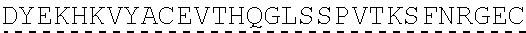 |
| Trastuzumab (CDR2H) Leptin | 43 | 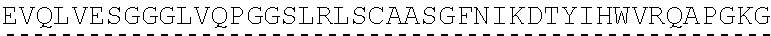 |
| | | 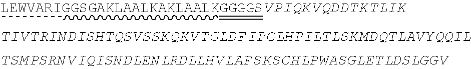 |
| | | 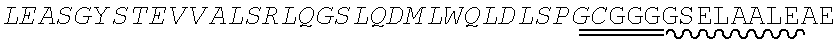 |
| | | 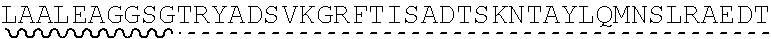 |
| | | 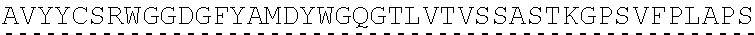 |
| | | 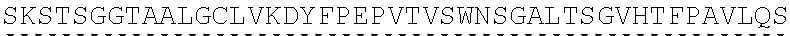 |
| | | 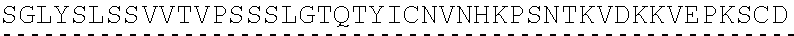 |
| | | 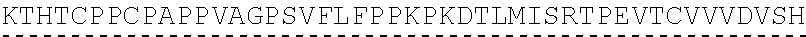 |
| | | 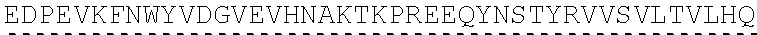 |
| | | 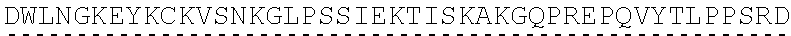 |
| | | 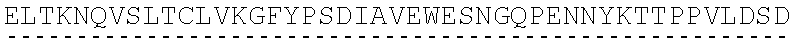 |
| | | 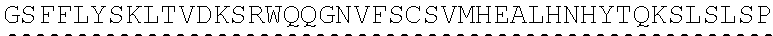 |
| | | 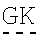 |
| Trastuzumab (CDR3H) Leptin | 44 | 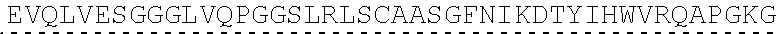 |
| | | 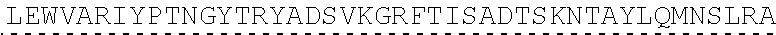 |
| | | 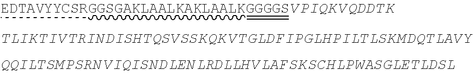 |
| | | 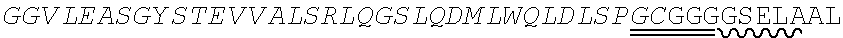 |
| | | 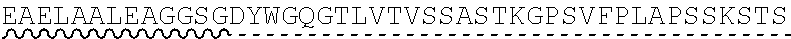 |
| | | 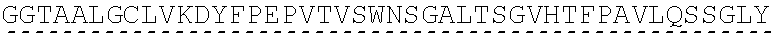 |
| | | 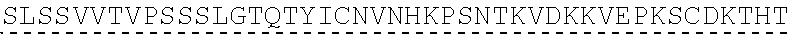 |
| | | 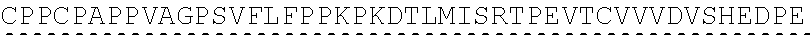 |
| | | 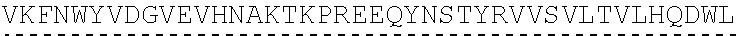 |
| | | 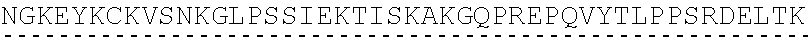 |
| | | 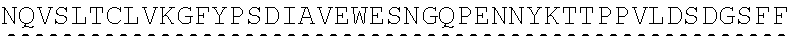 |
| | | 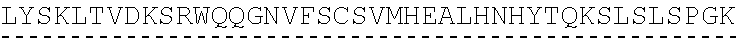 |
| Trastuzumab | 45 | 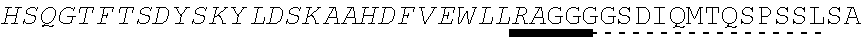 |
| (NL, GGGGS) | | 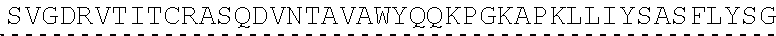 |
| ZP1 | | 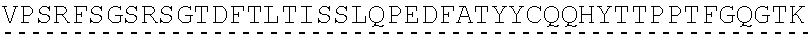 |
| | | 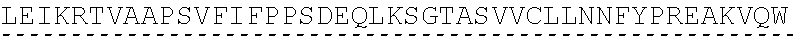 |
| | | 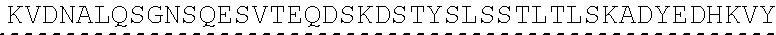 |
| | | 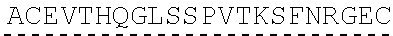 |
| Trastuzumab | 46 | 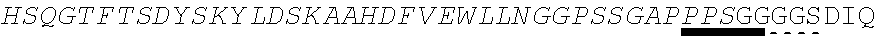 |
| (NL, GGGGS) | | 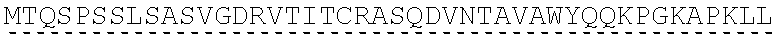 |
| ZPCEX | | 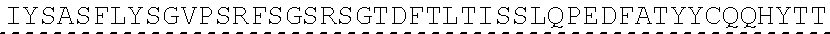 |
| | | 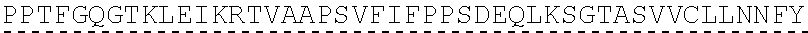 |
| | | 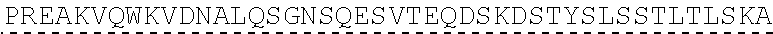 |
| | | 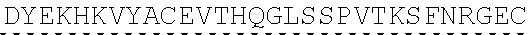 |
| Trastuzumab | 47 | 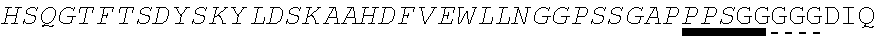 |
| (NL, GGGGG) | | 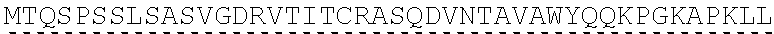 |
| ZPCEX | | 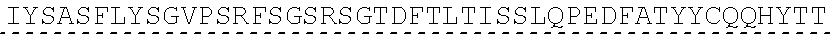 |
| | | 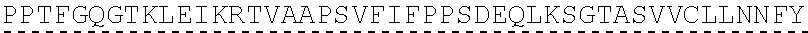 |
| | | 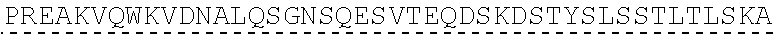 |
| | | 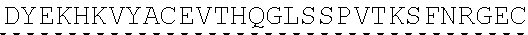 |
| Palivizumab | 48 | 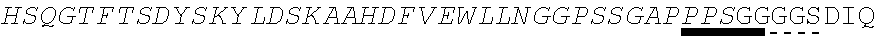 |
| (NL, GGGGS) | | 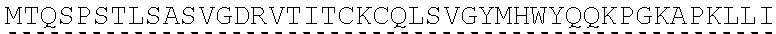 |
| ZPCEX | | 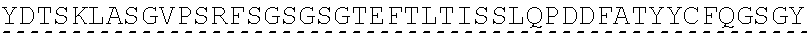 |
| | | 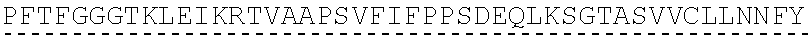 |
| | | 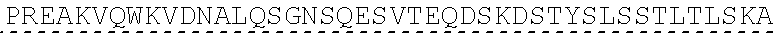 |
| | | 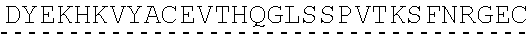 |
| Palivizumab | 49 | 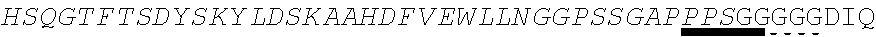 |
| (NL, GGGGG) | | 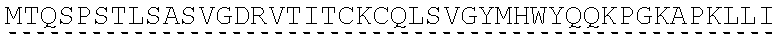 |
| ZPCEX | | 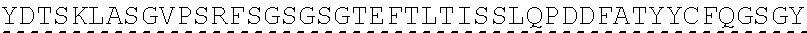 |
| | | 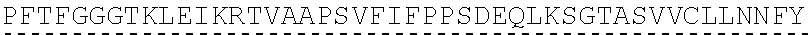 |
| | | 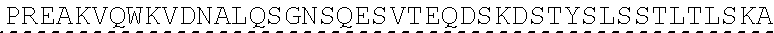 |
| | | 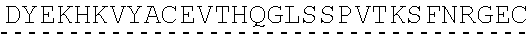 |
| Palivizumab | 50 | 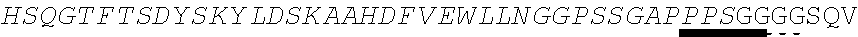 |
| (NH, GGGGS) | | 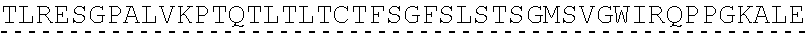 |
| ZPCEX | | 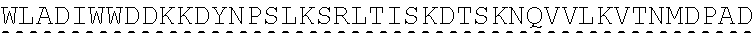 |
| | | 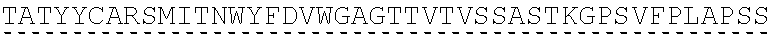 |
| | | 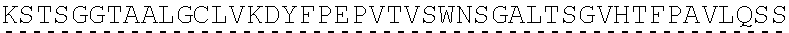 |
| | | 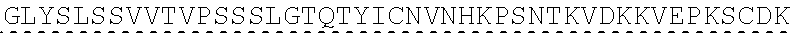 |
| | | 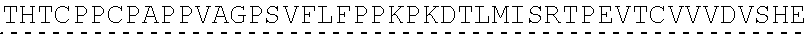 |
| | | 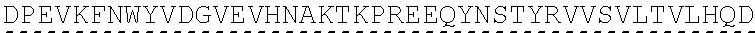 |
| | | 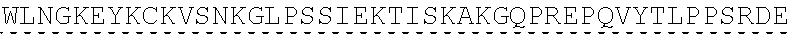 |
| | | 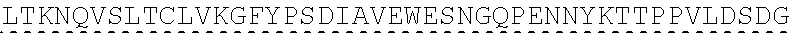 |
| | | 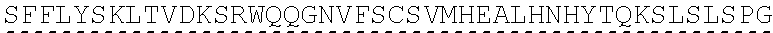 |
| | | 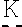 |
| Trastuzumab (NL, GGGGS) Relaxin2 | 51 | 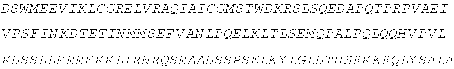 |
| | | 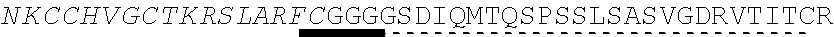 |
| | | 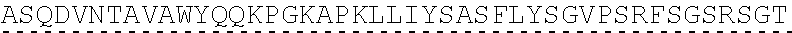 |
| | | 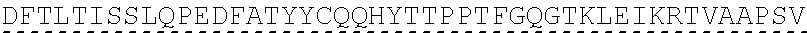 |
| | | 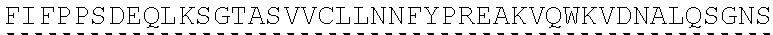 |
| | | 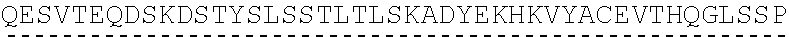 |
| | | 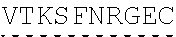 |
| Trastuzumab | 52 | 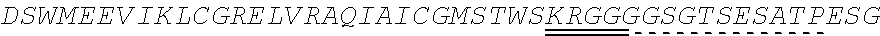 |
| (NL, GGGGS) | | 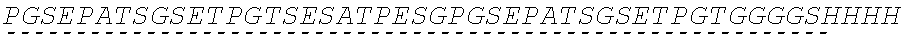 |
| Relaxin2 | | 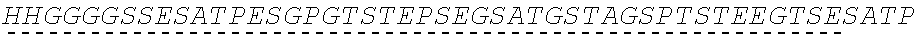 |
| (XT100) | | 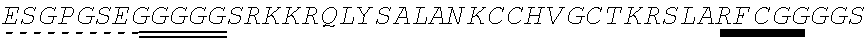 |
| | | 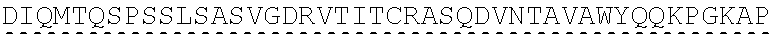 |
| | | 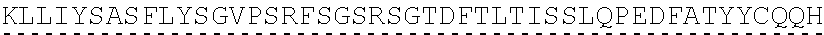 |
| | | 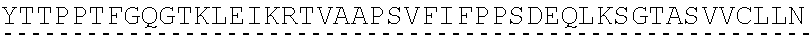 |
| | | 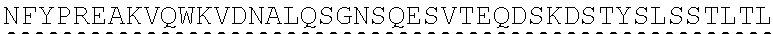 |
| | | 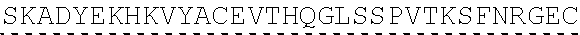 |
| Trastuzumab | 53 | 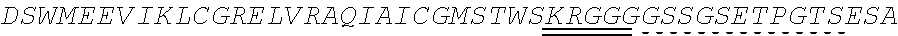 |
| (NL, GGGGS) | | 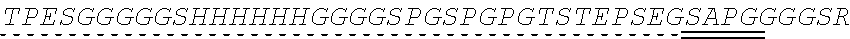 |
| Relaxin2 (XT35) | | 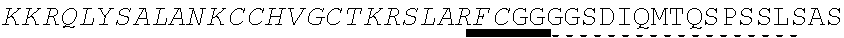 |
| | | 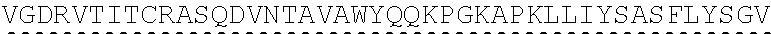 |
| | | 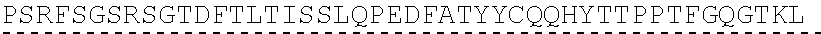 |
| | | 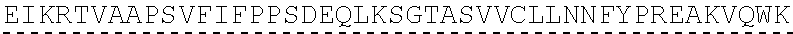 |
| | | 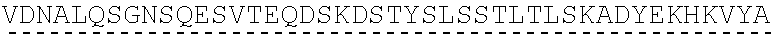 |
| | | 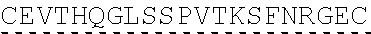 |
| Trastuzumab | 54 | 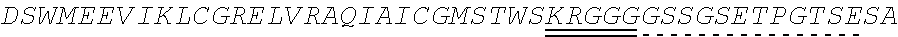 |
| (NL, GGGGG) | | 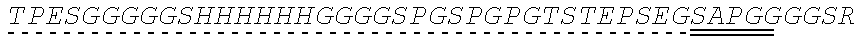 |
| Relaxin2 (XT35) | | 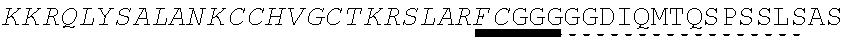 |
| | | 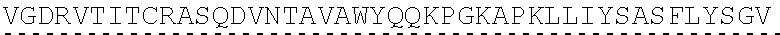 |
| | | 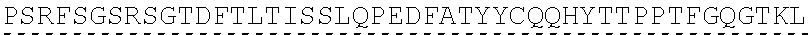 |
| | | 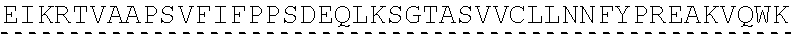 |
| | | 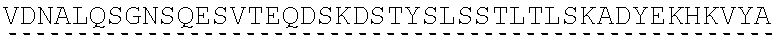 |
| | | 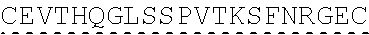 |
| Trastuzumab | 55 | 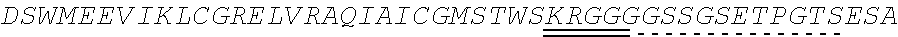 |
| (NL, CEXGGGGG) | | 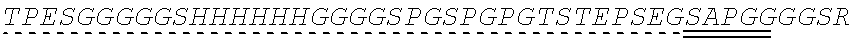 |
| Relaxin2 (XT35) | | 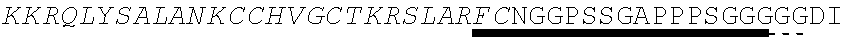 |
| | | 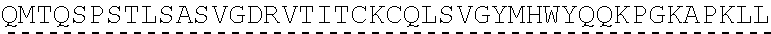 |
| | | 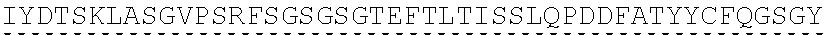 |
| | | 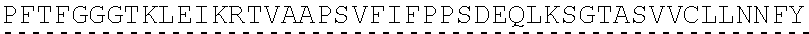 |
| | | 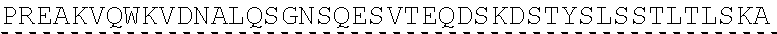 |
| | | 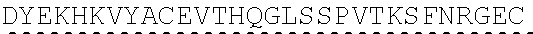 |
| Palivizumab | 56 | 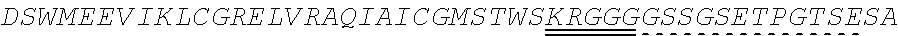 |
| (NL, GGGGS) | | 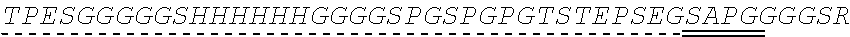 |
| Relaxin2 (XT35) | | 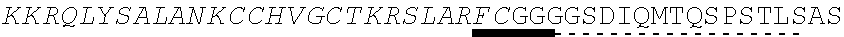 |
| | | 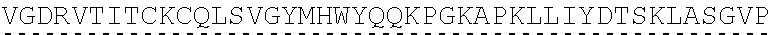 |
| | | 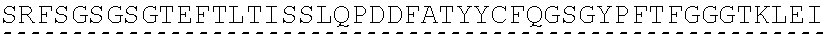 |
| | | 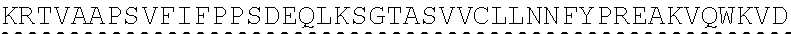 |
| | | 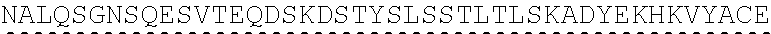 |
| | | 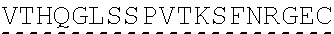 |
| Palivizumab | 57 | 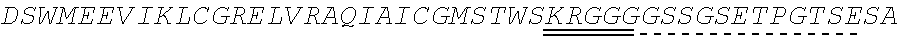 |
| (NL, GGGGG) | | 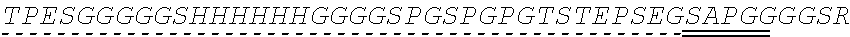 |
| Relaxin2 (XT35) | | 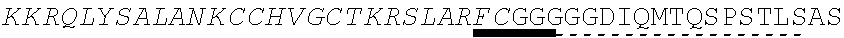 |
| | | 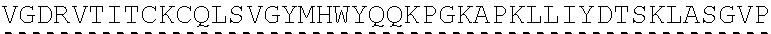 |
| | | 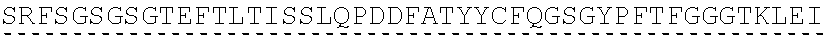 |
| | | 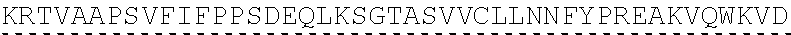 |
| | | 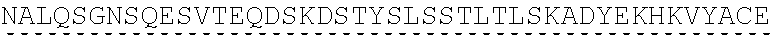 |
| | | 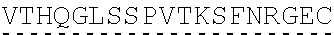 |
| Palivizumab | 58 | 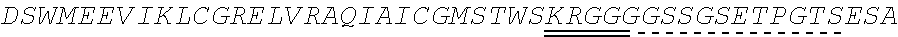 |
| (NL, CEXGGGGG) | | 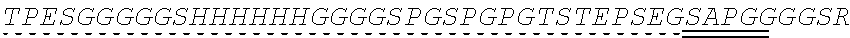 |
| Relaxin2 (XT35) | | 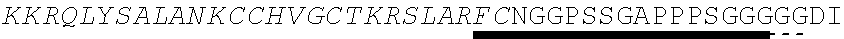 |
| | | 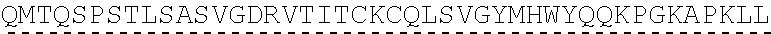 |
| | | 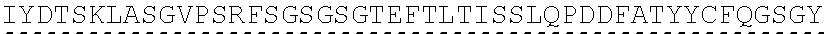 |
| | | 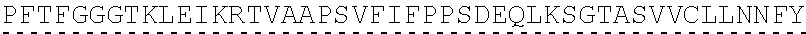 |
| | | 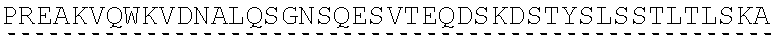 |
| | | 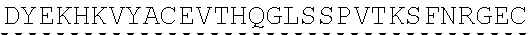 |
| Trastuzumab | 59 | 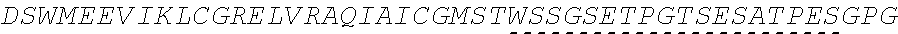 |
| (NL, GGGGS) | | 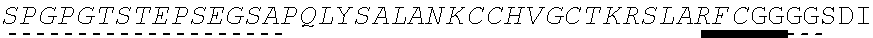 |
| Relaxin2 | | 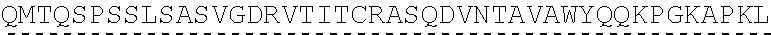 |
| (single) | | 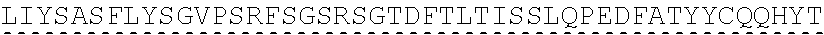 |
| | | 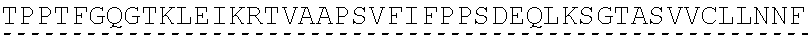 |
| | | 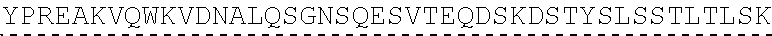 |
| | | 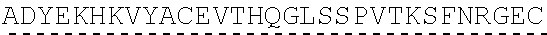 |
| Trastuzumab | 60 | 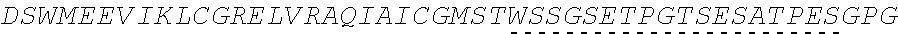 |
| (NL, GGGGG) | | 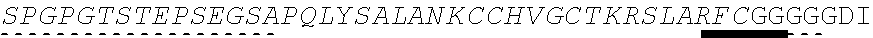 |
| Relaxin2 | | 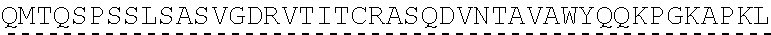 |
| (single) | | 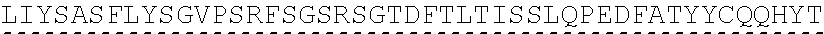 |
| | | 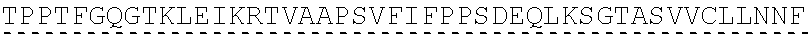 |
| | | 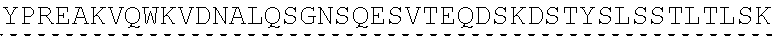 |
| | | 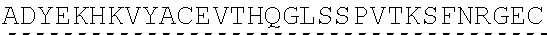 |
| Trastuzumab | 61 | 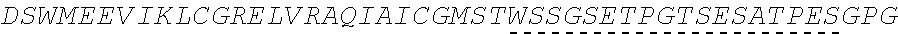 |
| (NL, CEXGGGGG) | | 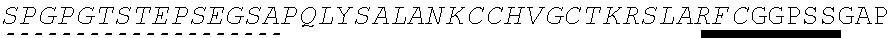 |
| Relaxin2 | | 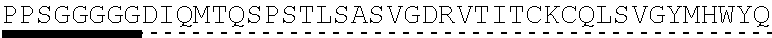 |
| (single) | | 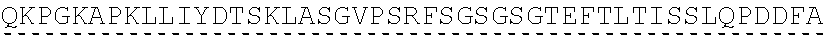 |
| | | 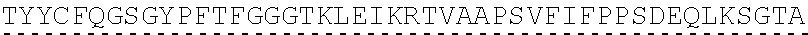 |
| | | 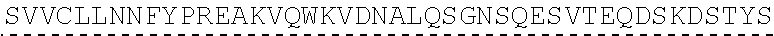 |
| | | 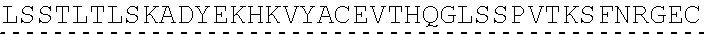 |
| Palivizumab | 62 | 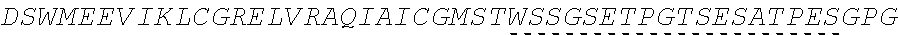 |
| (NL, GGGGS) | | 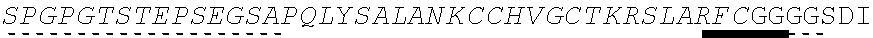 |
| Relaxin2 | | 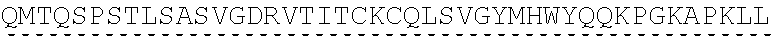 |
| (single) | | 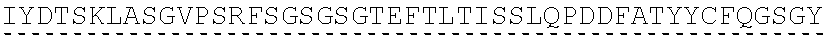 |
| | | 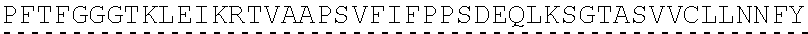 |
| | | 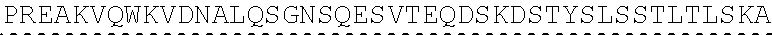 |
| | | 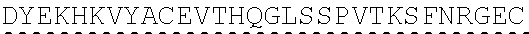 |
| Palivizumab | 63 | 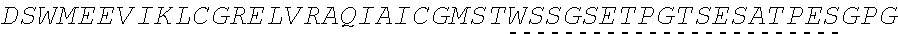 |
| (NL, GGGGG) | | 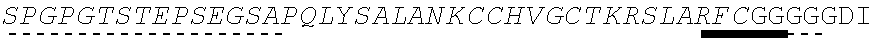 |
| Relaxin2 | | 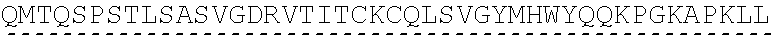 |
| (single) | | 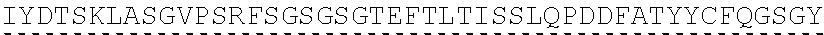 |
| | | 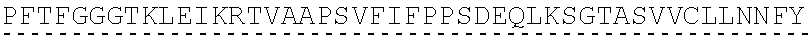 |
| | | 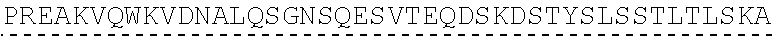 |
| | | 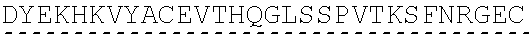 |
| Palivizumab | 64 | 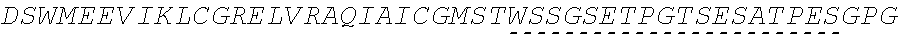 |
| (NL, CEXGGGGG) | | 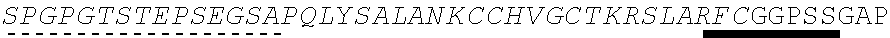 |
| Relaxin2 | | 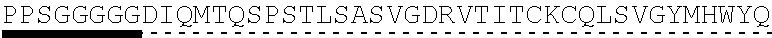 |
| (single) | | 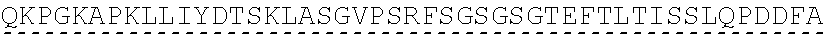 |
| | | 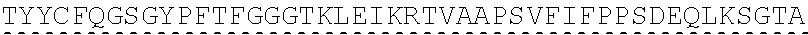 |
| | | 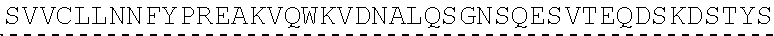 |
| | | 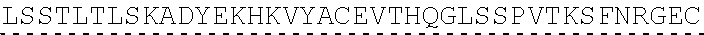 |
| Trastuzumab | 65 | 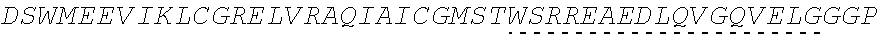 |
| (NL, GGGGS) | | 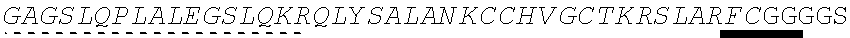 |
| Relaxin2 | | 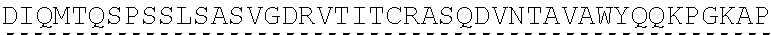 |
| (insulin C | | 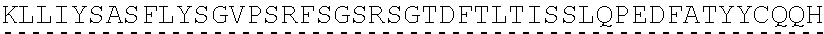 |
| peptide) | | 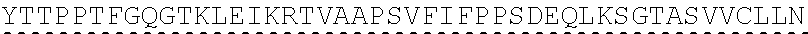 |
| | | 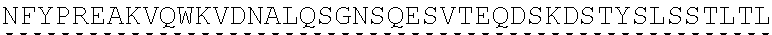 |
| | | 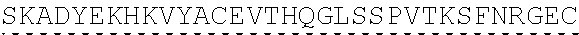 |
| Trastuzumab | 66 | 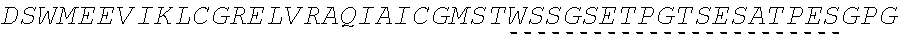 |
| (NL, GGGGS) | | 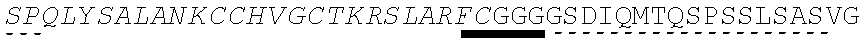 |
| Relaxin2 (XT21) | | 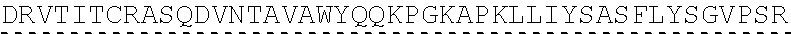 |
| | | 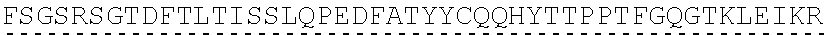 |
| | | 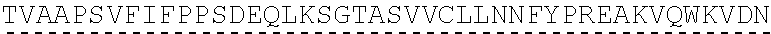 |
| | | 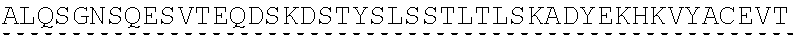 |
| | | 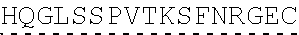 |
| Trastuzumab (NL, GGGGS) | 67 | 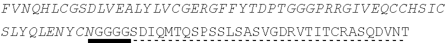 |
| Insulin | | 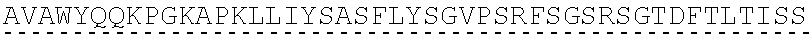 |
| | | 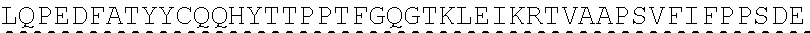 |
| | | 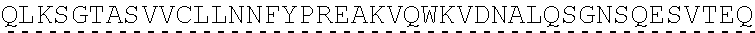 |
| | | 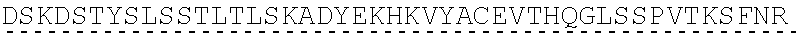 |
| | | 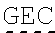 |
| Trastuzumab | 68 | 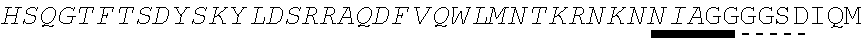 |
| (NL, GGGGS) | | 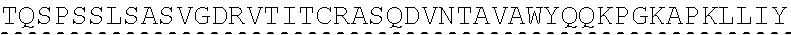 |
| Oxyntomodulin | | 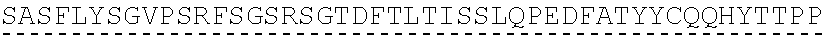 |
| | | 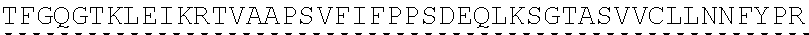 |
| | | 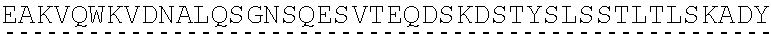 |
| | | 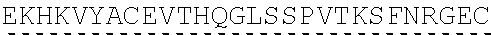 |
| Palivizumab | 69 | 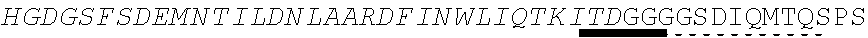 |
| (NL, GGGGS) | | 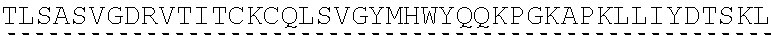 |
| GLP2 | | 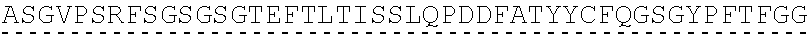 |
| | | 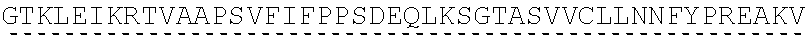 |
| | | 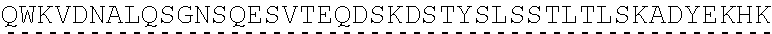 |
| | | 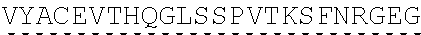 |
| Palivizumab | 70 | 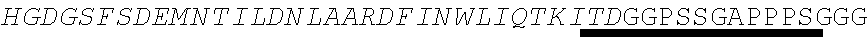 |
| (NL, CEXGGGGS) | | 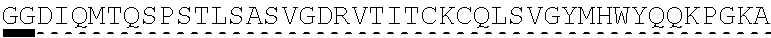 |
| GLP2 | | 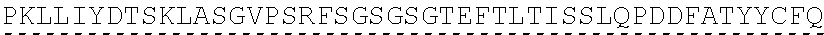 |
| | | 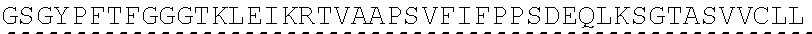 |
| | | 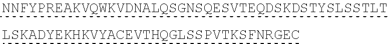 |
| Trastuzumab | 71 | 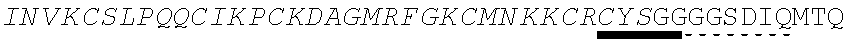 |
| (NL, GGGGS) | | 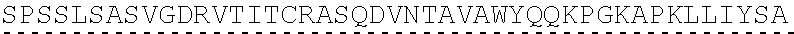 |
| Moka | | 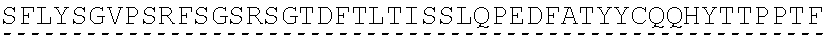 |
| | | 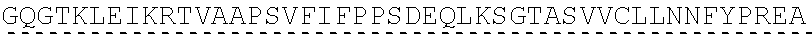 |
| | | 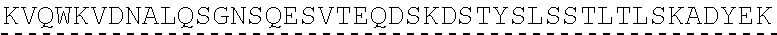 |
| | | 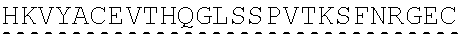 |
| Trastuzumab (NL, GGGGS) | 72 | 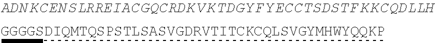 |
| Ssam6 | | 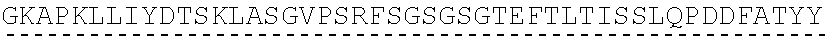 |
| | | 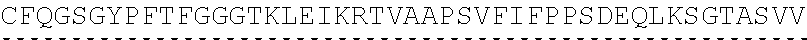 |
| | | 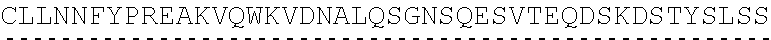 |
| | | 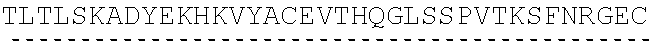 |
| Trastuzumab | 73 | 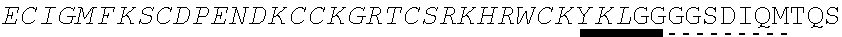 |
| (NL, GGGGS) | | 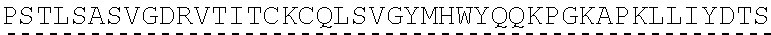 |
| 550 | | 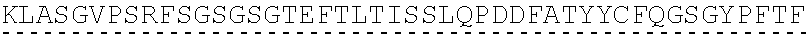 |
| | | 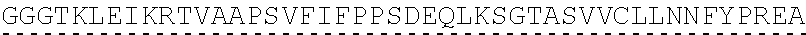 |
| | | 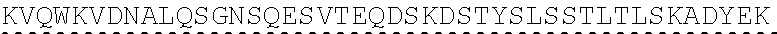 |
| | | 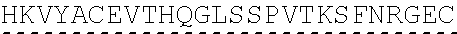 |
| Trastuzumab (NL, GGGGS) | 74 | 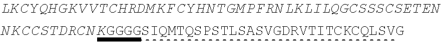 |
| Mambalign 1 | | 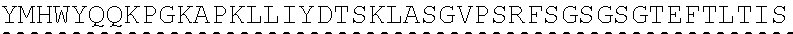 |
| | | 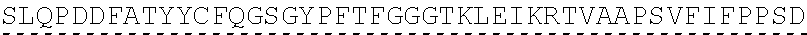 |
| | | 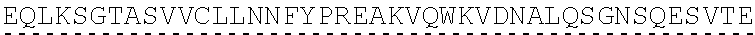 |
| | | 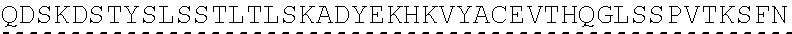 |
| | | 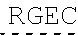 |
| Palivizumab (NH, GGGGS) | 192 | 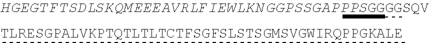 |
| Exendin-4 | | 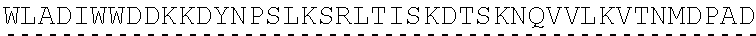 |
| | | 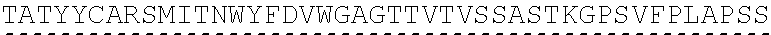 |
| | | 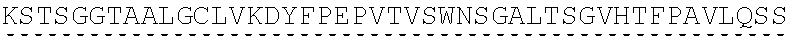 |
| | | 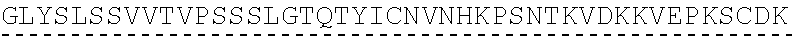 |
| | | 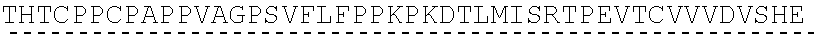 |
| | | 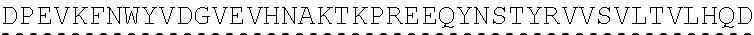 |
| | | 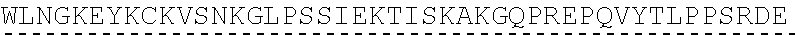 |
| | | 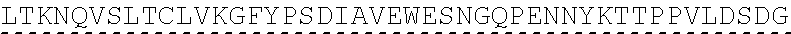 |
| | | 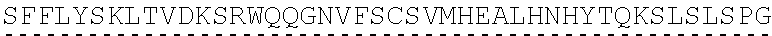 |
| | | 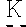 |
| Palivizumab (NL, EAAAK) | 193 | 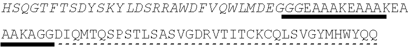 |
| Glucagon | | 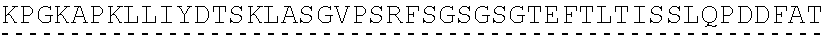 |
| | | 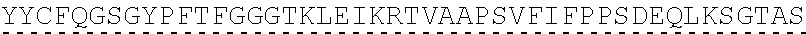 |
| | | 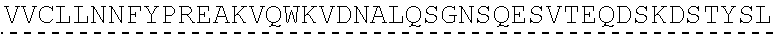 |
| | | 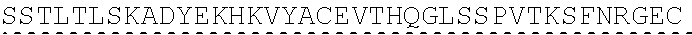 |
| Palivizumab | 194 | 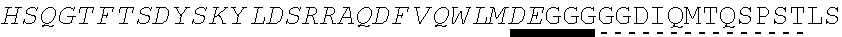 |
| (NL, GGGGG) | | 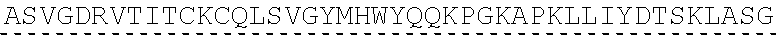 |
| Glucagon | | 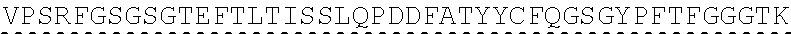 |
| | | 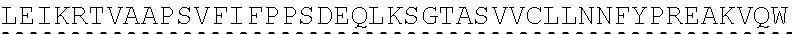 |
| | | 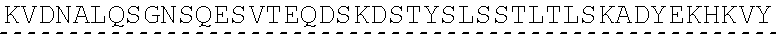 |
| | | 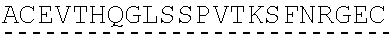 |
| Palivizumab (NH, EAAAK) | 195 | 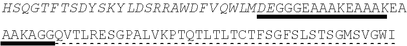 |
| Glucagon | | 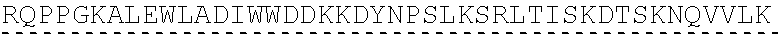 |
| | | 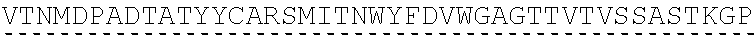 |
| | | 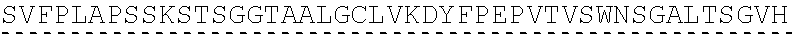 |
| | | 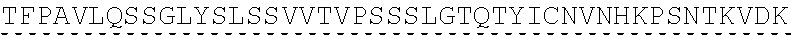 |
| | | 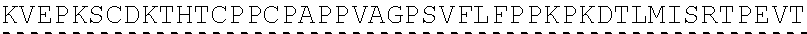 |
| | | 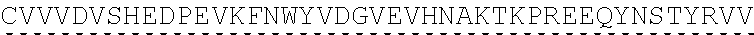 |
| | | 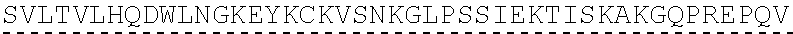 |
| | | 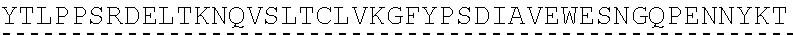 |
| | | 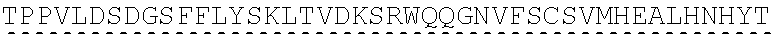 |
| | | 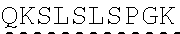 |
| Palivizumab (NH, EAAAK) | 196 | 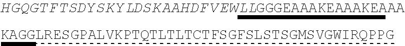 |
| ZP1 | | 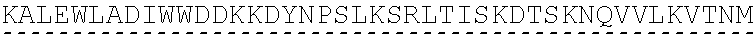 |
| | | 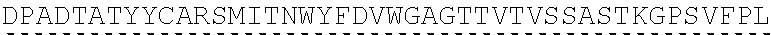 |
| | | 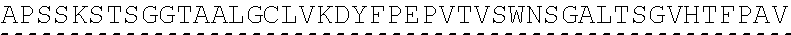 |
| | | 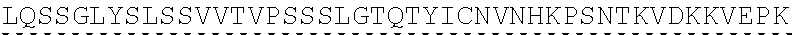 |
| | | 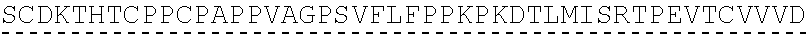 |
| | | 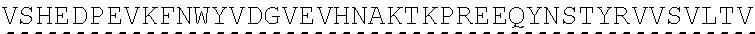 |
| | | 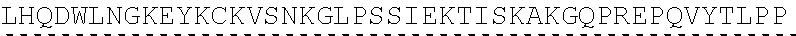 |
| | | 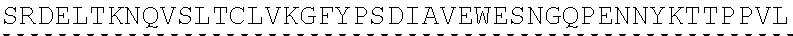 |
| | | 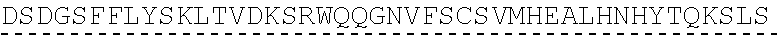 |
| | | 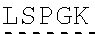 |
| Trastuzumab | 197 | 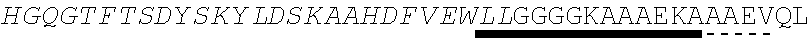 |
| (NH, EAAAK) | | 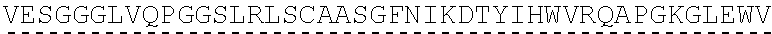 |
| ZP1 | | 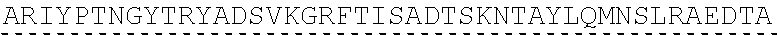 |
| | | 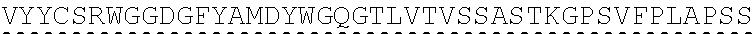 |
| | | 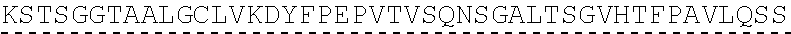 |
| | | 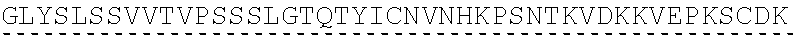 |
| | | 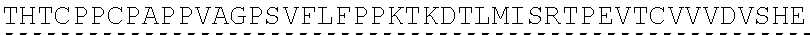 |
| | | 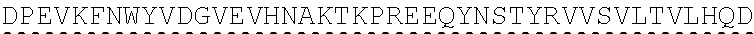 |
| | | 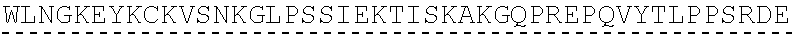 |
| | | 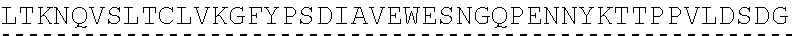 |
| | | 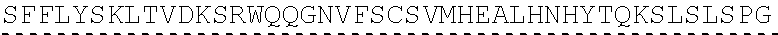 |
| | | 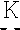 |
| Trastuzumab (NH, XT21) | 198 | 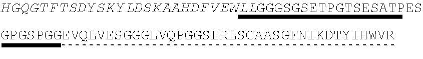 |
| ZP1 | | 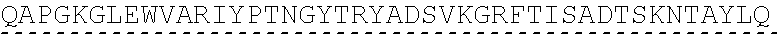 |
| | | 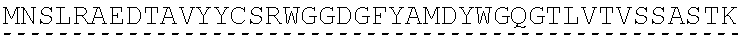 |
| | | 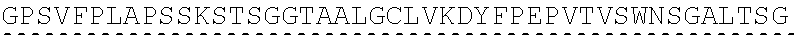 |
| | | 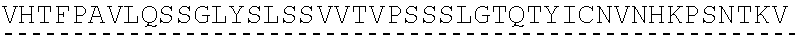 |
| | | 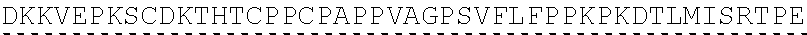 |
| | | 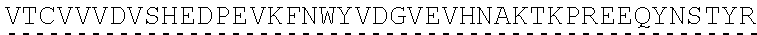 |
| | | 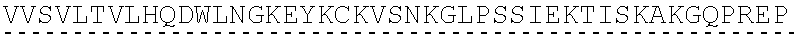 |
| | | 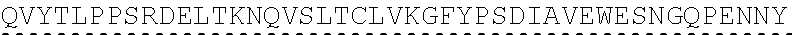 |
| | | 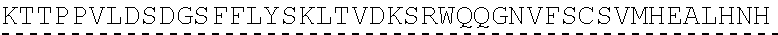 |
| | | 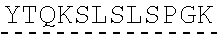 |
| Palivizumab (NL, EAAAK) | 199 | 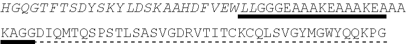 |
| ZP1 | | 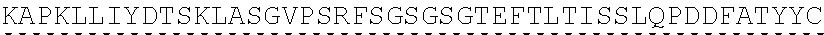 |
| | | 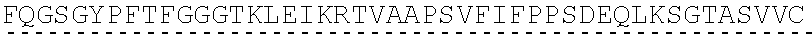 |
| | | 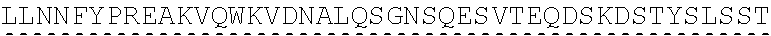 |
| | | 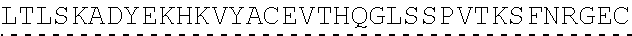 |
| Palivizumab (NL, XT21) | 200 | 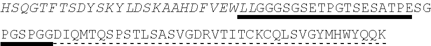 |
| ZP1 | | 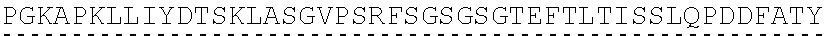 |
| | | 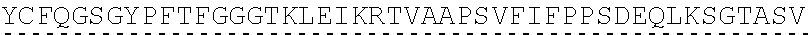 |
| | | 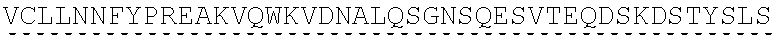 |
| | | 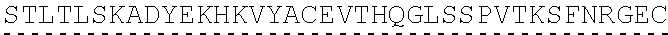 |
| Palivizumab | 201 | 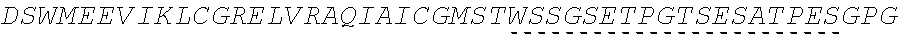 |
| (NH, CEXGGGGG) | | 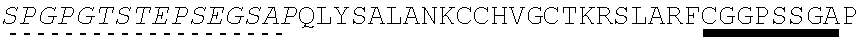 |
| Relaxin2 | | 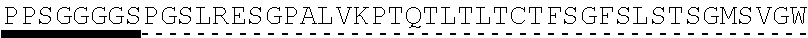 |
| (single) | | 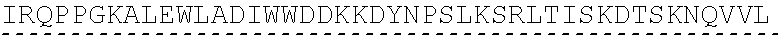 |
| | | 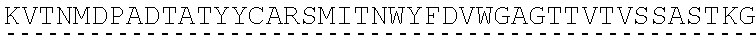 |
| | | 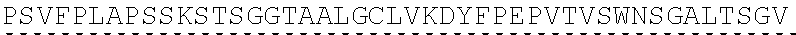 |
| | | 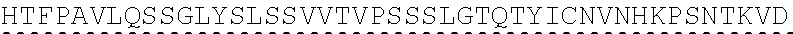 |
| | | 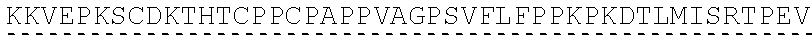 |
| | | 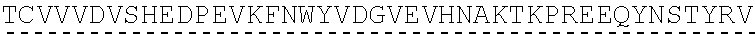 |
| | | 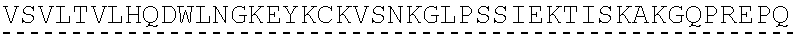 |
| | | 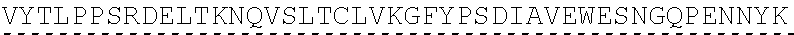 |
| | | 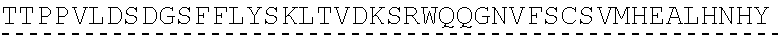 |
| | | 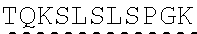 |
| Palivizumab (NH, EAAAK) | 202 | 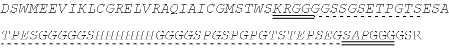 |
| Relaxin2 (XT35) | | 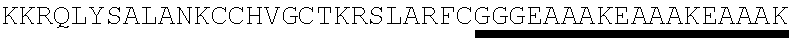 |
| | | 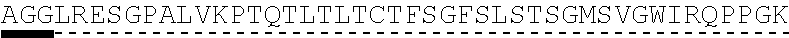 |
| | | 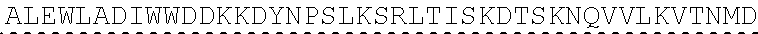 |
| | | 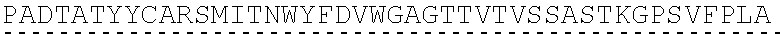 |
| | | 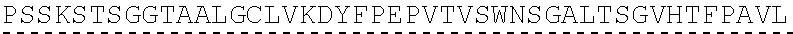 |
| | | 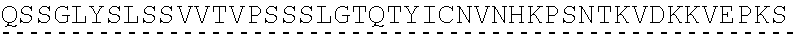 |
| | | 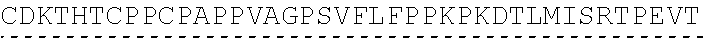 |
| | | 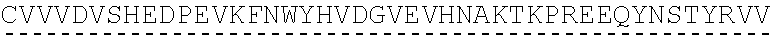 |
| | | 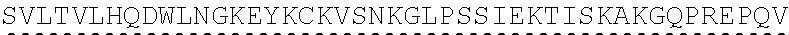 |
| | | 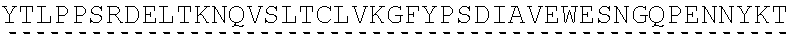 |
| | | 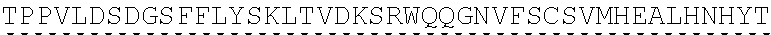 |
| | | 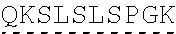 |
| Palivizumab Fab | 203 | 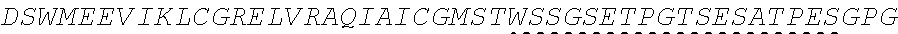 |
| (NH, CEXGGGGS) | | 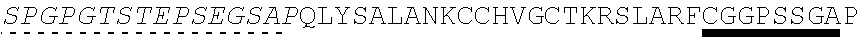 |
| Relaxin2 | | 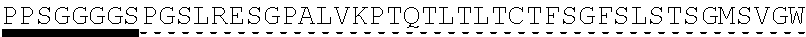 |
| (single) | | 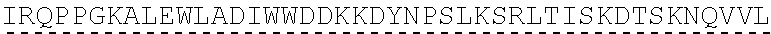 |
| | | 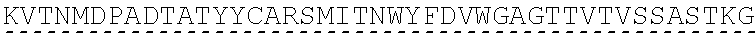 |
| | | 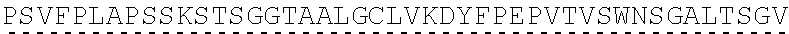 |
| | | 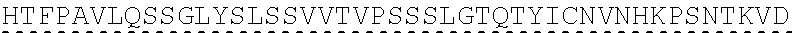 |
| | | 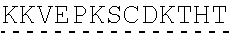 |
| Palivizumab (NH, CEXGGGGG) | 204 | 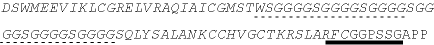 |
| Relaxin2 (30GS) | | 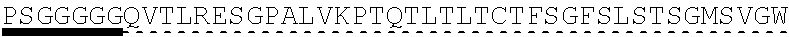 |
| | | 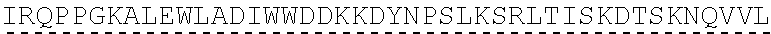 |
| | | 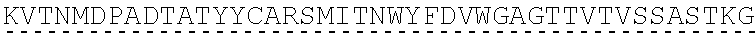 |
| | | 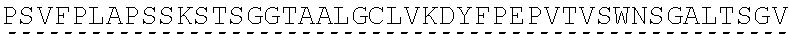 |
| | | 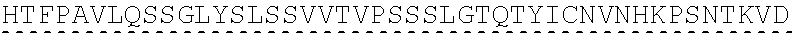 |
| | | 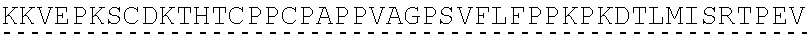 |
| | | 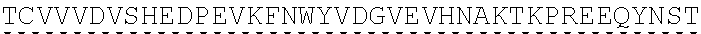 |
| | | 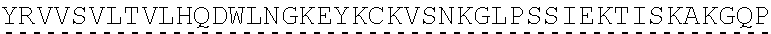 |
| | | 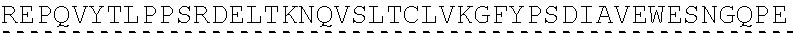 |
| | | 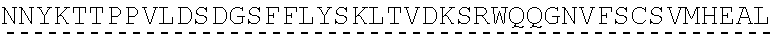 |
| | | 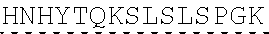 |
| Palivizumab | 205 | 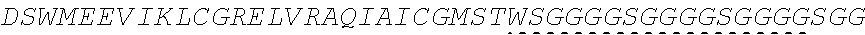 |
| (NH, CEXGGGGG) | | 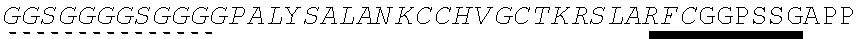 |
| Relaxin2 Q60A | | 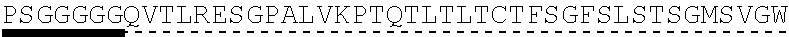 |
| (30GS) | | 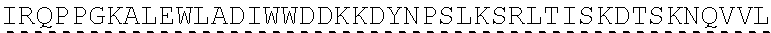 |
| | | 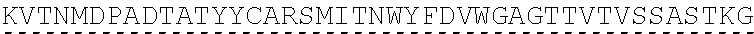 |
| | | 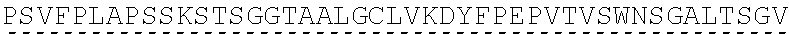 |
| | | 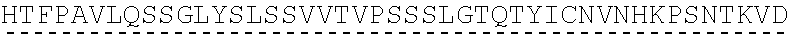 |
| | | 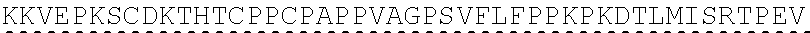 |
| | | 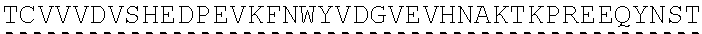 |
| | | 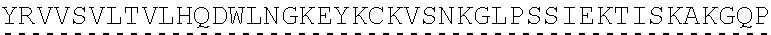 |
| | | 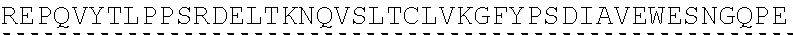 |
| | | 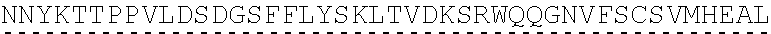 |
| | | 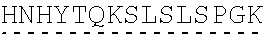 |
| Palivizumab | 206 | 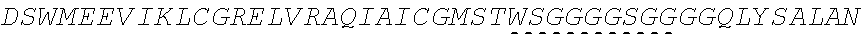 |
| (NH, CEXGGGGG) | | 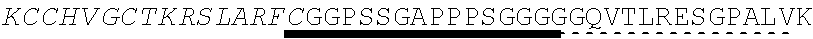 |
| Relaxin2 (9GS) | | 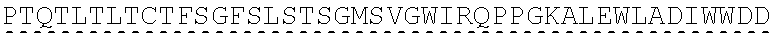 |
| | | 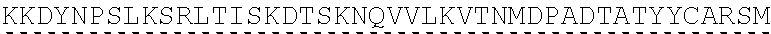 |
| | | 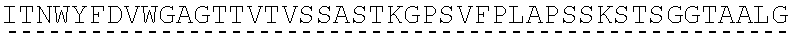 |
| | | 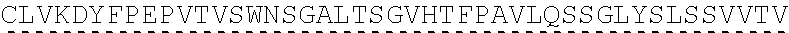 |
| | | 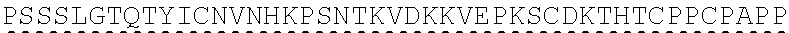 |
| | | 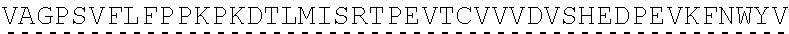 |
| | | 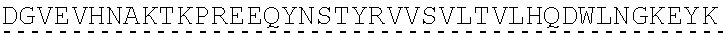 |
| | | 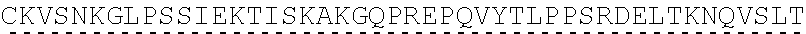 |
| | | 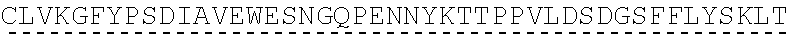 |
| | | 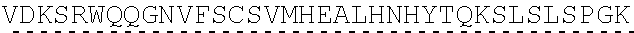 |
| Palivizumab | 207 | 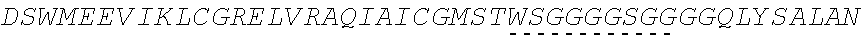 |
| (NH, GGGGS) | | 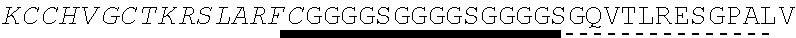 |
| Relaxin2c (9GS) | | 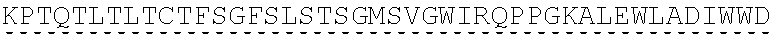 |
| | | 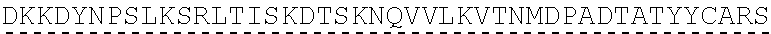 |
| | | 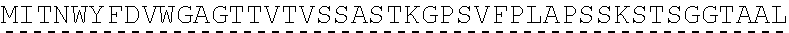 |
| | | 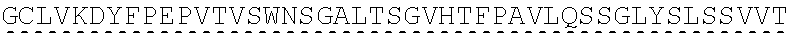 |
| | | 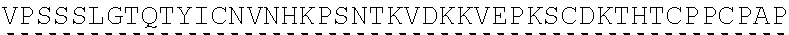 |
| | | 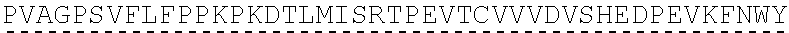 |
| | | 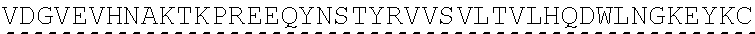 |
| | | 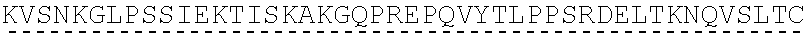 |
| | | 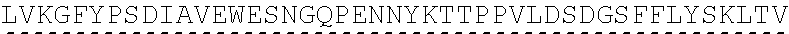 |
| | | 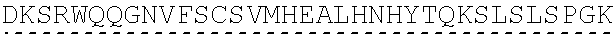 |
| Palivizumab Fab | 208 | 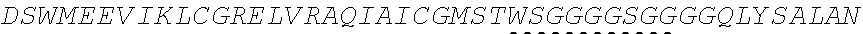 |
| (NH, CEXGGGGG) | | 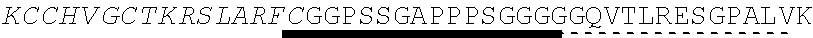 |
| Relaxin2 (9GS) | | 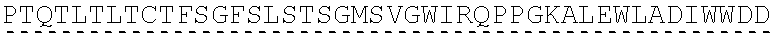 |
| | | 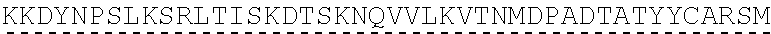 |
| | | 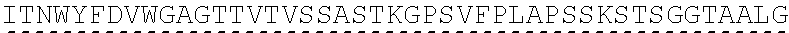 |
| | | 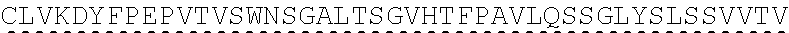 |
| | | 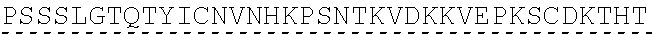 |
| Palivizumab Fab | 209 | 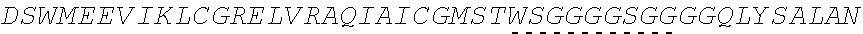 |
| (NH, GGGGS) | | 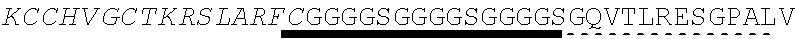 |
| Relaxin2c (9GS) | | 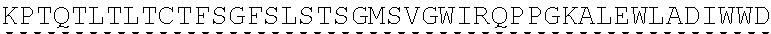 |
| | | 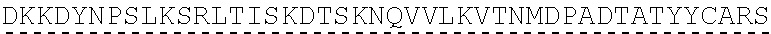 |
| | | 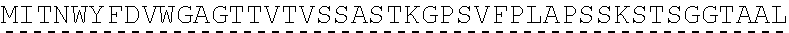 |
| | | 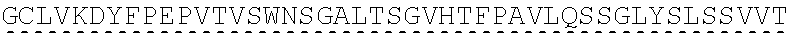 |
| | | 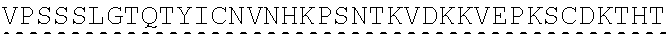 |
| Palivizumab | 210 | 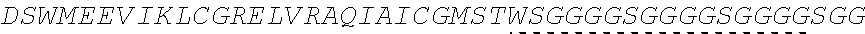 |
| (NH, GGGGS) | | 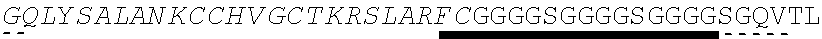 |
| Relaxin2 (18GS) | | 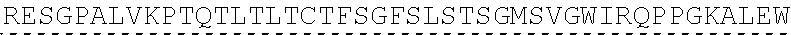 |
| | | 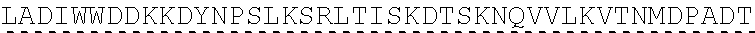 |
| | | 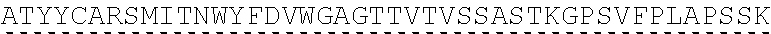 |
| | | 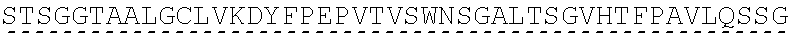 |
| | | 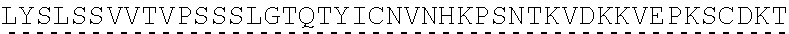 |
| | | 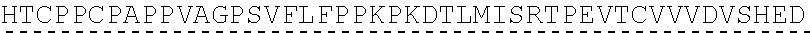 |
| | | 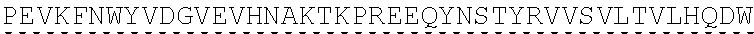 |
| | | 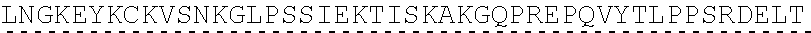 |
| | | 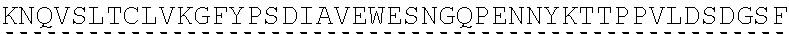 |
| | | 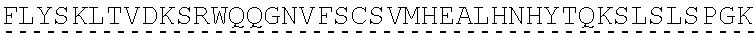 |
| Palivizumab (NH, GGGG) Relaxin2 (GGGPRR) | 211 | 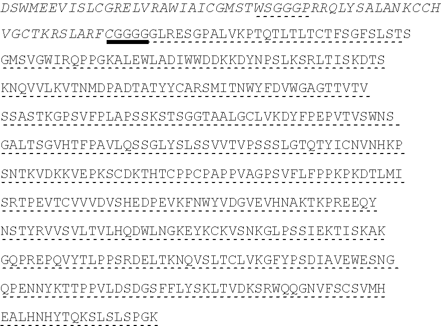 |
| Palivizumab (NH, CEXGGGGG) Relaxin2 (GGGPRR) | 212 | 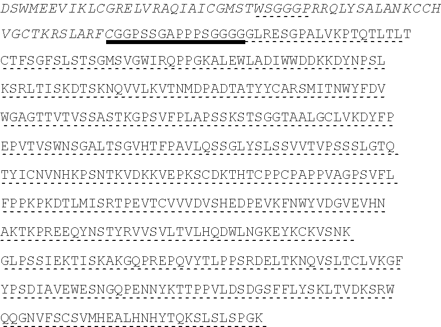 |
| Palivizumab | 213 | 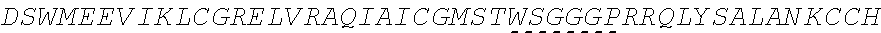 |
| (NH, EAAAK) | | 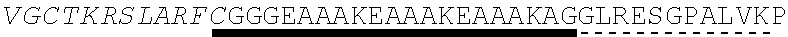 |
| Relaxin2 | | 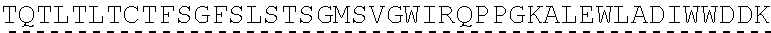 |
| (GGGPRR) | | 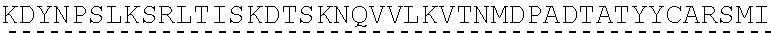 |
| | | 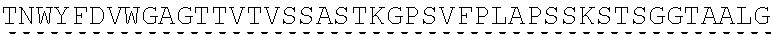 |
| | | 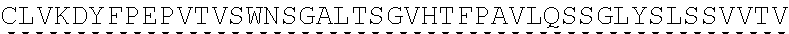 |
| | | 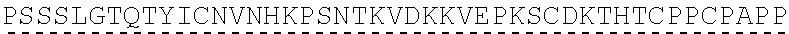 |
| | | 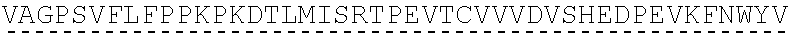 |
| | | 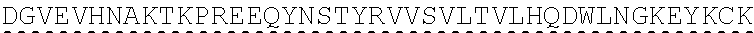 |
| | | 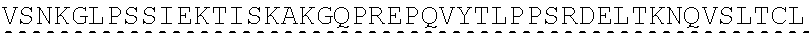 |
| | | 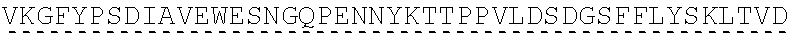 |
| | | 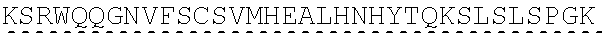 |
| Palivizumab | 214 | 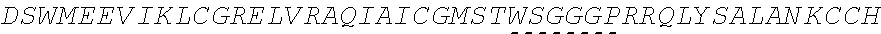 |
| (NL, CEXGGGGG) | | 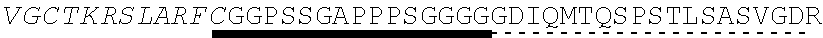 |
| Relaxin2 | | 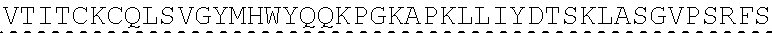 |
| (GGGPRR) | | 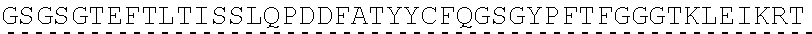 |
| | | 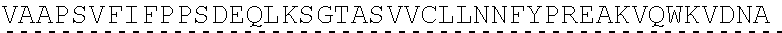 |
| | | 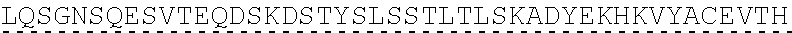 |
| | | 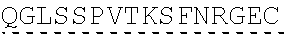 |
| Palivizumab | 215 | 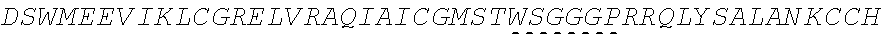 |
| (NL, EAAAK) | | 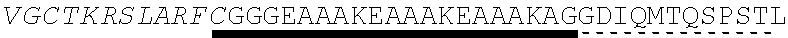 |
| Relaxin2 | | 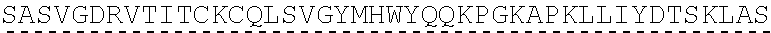 |
| (GGGPRR) | | 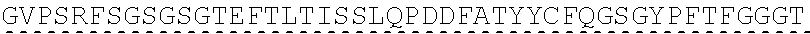 |
| | | 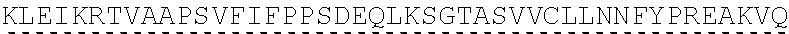 |
| | | 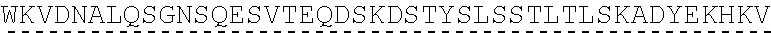 |
| | | 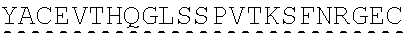 |
| Palivizumab | 216 | 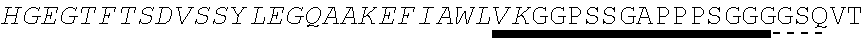 |
| (NH, CEXGGGGS) | | 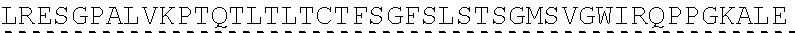 |
| GLP1 | | 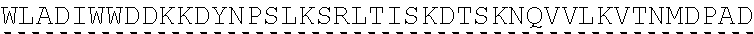 |
| | | 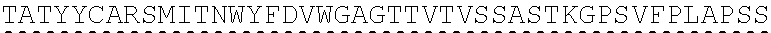 |
| | | 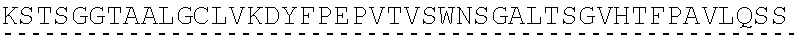 |
| | | 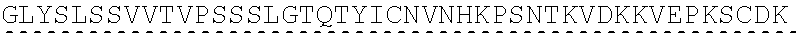 |
| | | 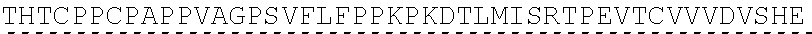 |
| | | 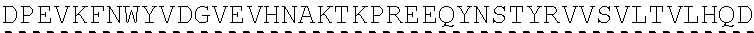 |
| | | 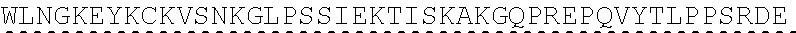 |
| | | 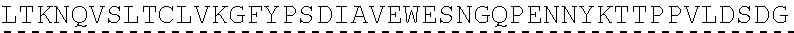 |
| | | 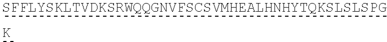 |
| Palivizumab | 217 | 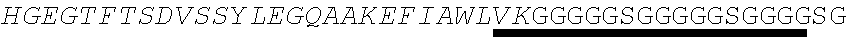 |
| (NH, GGGGS) | | 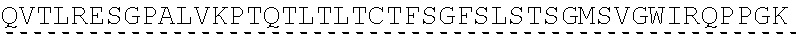 |
| GLP1 | | 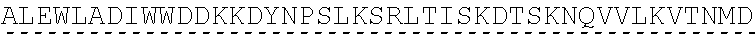 |
| | | 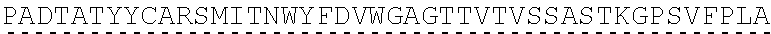 |
| | | 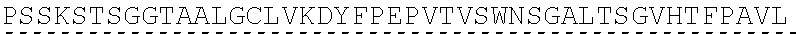 |
| | | 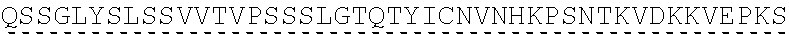 |
| | | 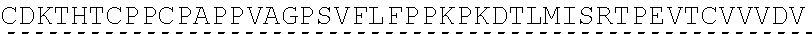 |
| | | 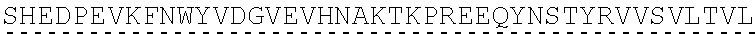 |
| | | 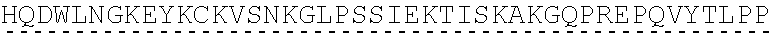 |
| | | 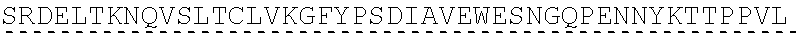 |
| | | 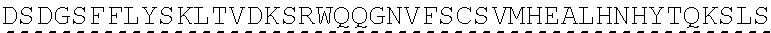 |
| | | 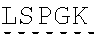 |
| Palivizumab | 218 | 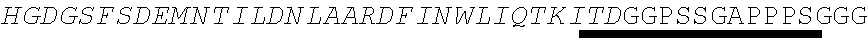 |
| (NH, CEXGGGGS) | | 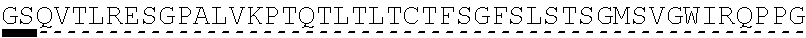 |
| GLP2 | | 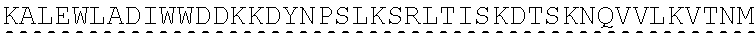 |
| | | 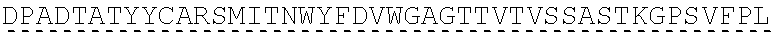 |
| | | 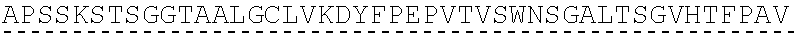 |
| | | 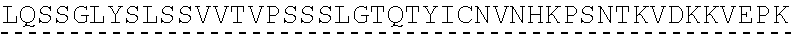 |
| | | 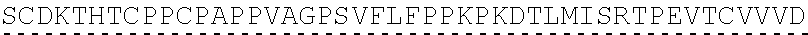 |
| | | 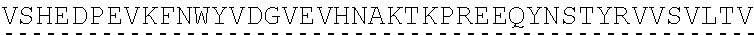 |
| | | 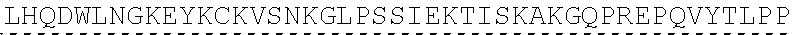 |
| | | 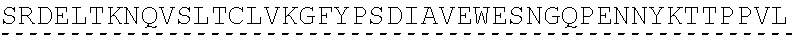 |
| | | 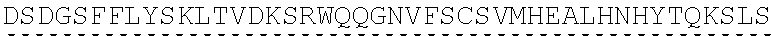 |
| | | 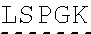 |
| Palivizumab | 219 | 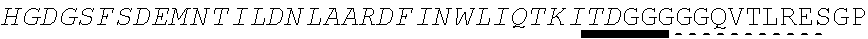 |
| (NH, GGGGG) | | 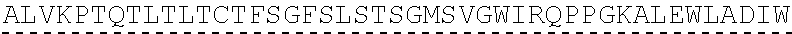 |
| GLP2 | | 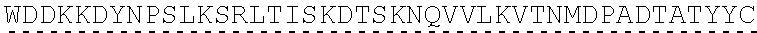 |
| | | 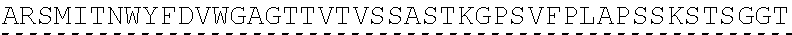 |
| | | 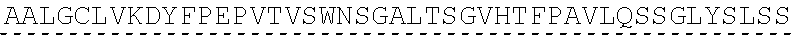 |
| | | 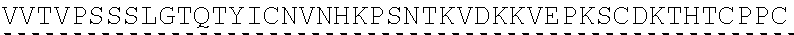 |
| | | 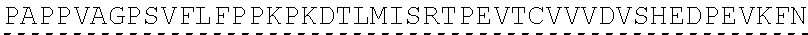 |
| | | 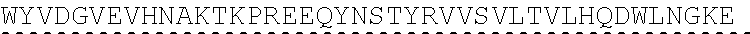 |
| | | 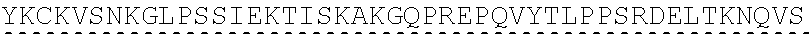 |
| | | 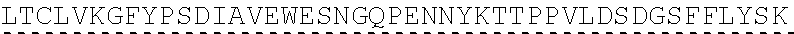 |
| | | 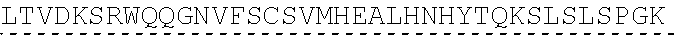 |
| Palivizumab | 220 | 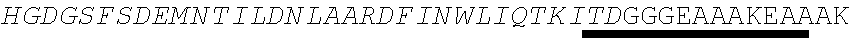 |
| (NH, EAAAK) | | 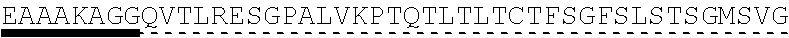 |
| GLP2 | | 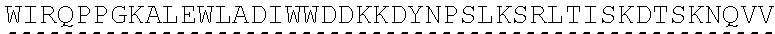 |
| | | 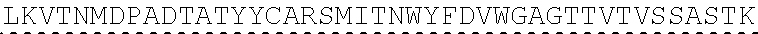 |
| | | 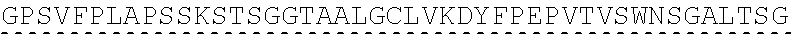 |
| | | 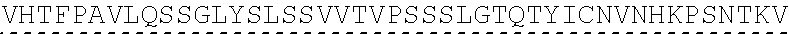 |
| | | 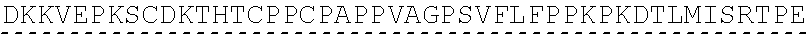 |
| | | 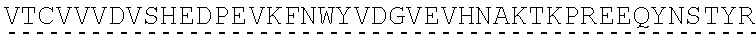 |
| | | 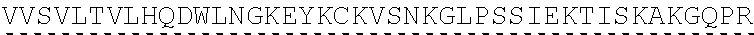 |
| | | 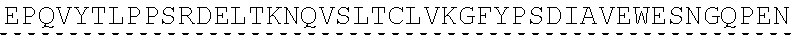 |
| | | 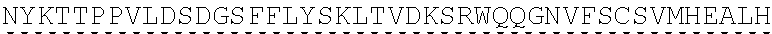 |
| | | 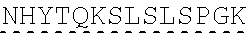 |
| Palivizumab | 221 | 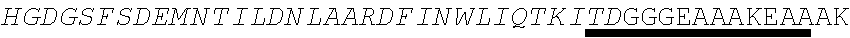 |
| (NL, EAAAK) | | 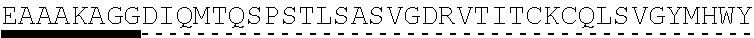 |
| GLP2 | | 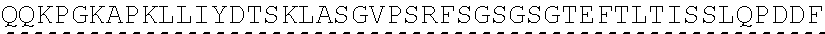 |
| | | 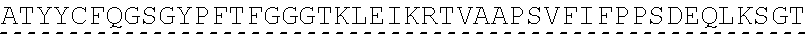 |
| | | 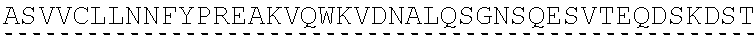 |
| | | 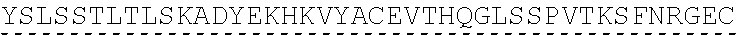 |
| Palivizumab | 222 | 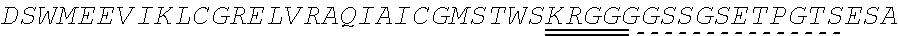 |
| (NH, EAAAK) | | 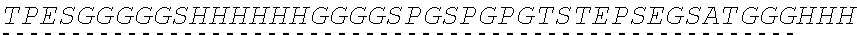 |
| Relaxin (dual) | | 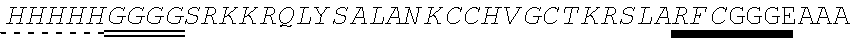 |
| | | 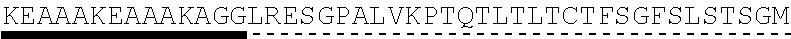 |
| | | 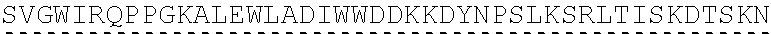 |
| | | 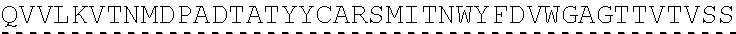 |
| | | 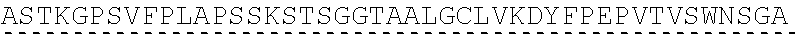 |
| | | 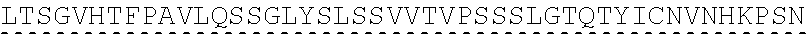 |
| | | 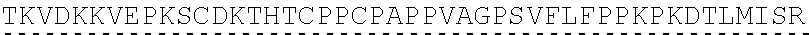 |
| | | 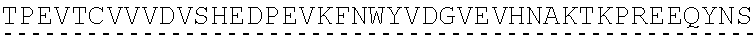 |
| | | 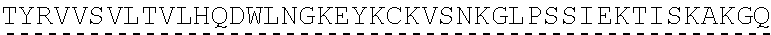 |
| | | 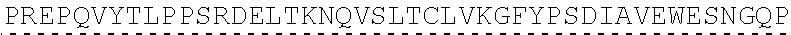 |
| | | 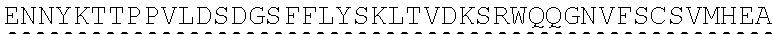 |
| | | 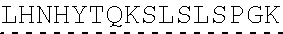 |
| Palivizumab | 266 | 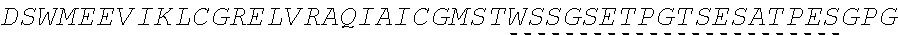 |
| (NH, EAAAK) | | 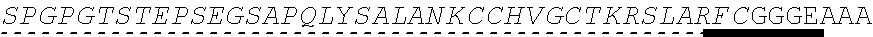 |
| Relaxin2 | | 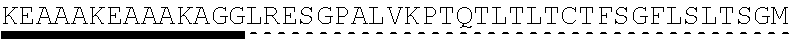 |
| (single) | | 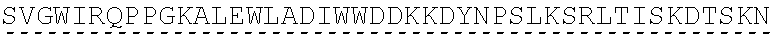 |
| | | 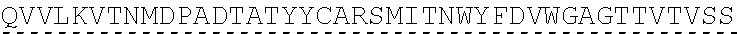 |
| | | 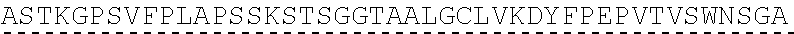 |
| | | 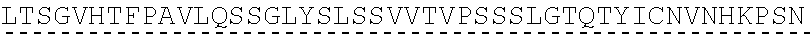 |
| | | 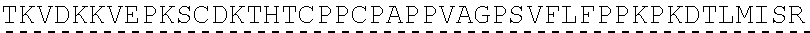 |
| | | 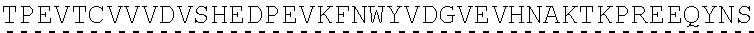 |
| | | 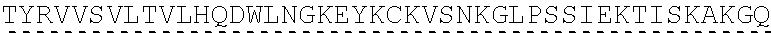 |
| | | 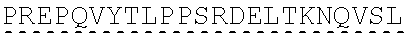 |

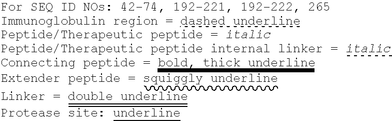

TABLE 5
Therapeutic Peptides—Nucleotide Sequence

| NAME | SEQ ID NO | SEQUENCE |
|---|---|---|
| Exendin-4 | 75 | CACGGAGAAGGAACATTTACCAGCGACCTCAGCAAGCAGATGGAG |
| | | GAAGAGGCCGTGAGGCTGTTCATCGAGTGGCTGAAGAACGGCGG |
| | | ACCCTCCTCTGGCGCTCCACCCCCTAGC |
| Leptin | 76 | GTTCCAATTCAAAAGGTTCAAGATGATACCAAAACTCTGATTAAAAC |
| | | TATTGTCACGCGTATAAACGACATCTCACATACCCAGTCGGTTAGC |
| | | TCAAAGCAAAAAGTTACCGGTTTGGACTTTATTCCGGGACTGCACC |
| | | CGATCCTGACCCTTAGTAAAATGGACCAGACACTGGCCGTCTACCA |
| | | GCAAATCCTGACATCGATGCCATCCAGAAATGTGATACAAATTAGC |
| | | AACGATTTGGAAAACCTTCGCGATCTGCTGCACGTGCTGGCCTTCA |
| | | GTAAGTCCTGTCATCTGCCGTGGGCGTCGGGACTGGAGACTCTTG |
| | | ACTCGCTGGGTGGAGTGTTAGAGGCCTCTGGCTATTCTACTGAAGT |
| | | CGTTGCGCTGTCACGCCTCCAGGGGAGCCTGCAGGACATGCTGTG |
| | | GCAGCTGGACCTGTCACCTGGCTGC |
| ZP1 | 77 | CACAGCCAGGGCACATTCACTAGCGATTATAGTAAATATCTGGATT |
| | | CCAAGGCAGCGCACGATTTTGTAGAGTGGCTCTTGC |
| ZPCEX | 78 | CACAGCCAGGGCACATTCACTAGCGATTATAGTAAATATCTGGATT |
| | | CCAAGGCAGCGCACGATTTTGTAGAGTGGCTCTTGAACGGAGGCC |
| | | CTTCCTCCGGAGCTCCACCTCCGTCC |
| Relaxin2 | 79 | GACTCTTGGATGGAAGAAGTTATCAAACTGTGCGGTCGTGAACTGG |
| | | TTCGTGCTCAGATCGCTATCTGCGGTATGTCTACCTGGTCTAAACG |
| | | TTCTCTGTCTCAGGAAGACGCTCCGCAGACCCCGCGTCCGGTTGC |
| | | TGAAATCGTTCCGTCTTTCATCAACAAAGACACCGAAACCATCAACA |
| | | TGATGTCTGAATTCGTTGCTAACCTGCCGCAGGAACTGAAACTGAC |
| | | CCTGTCTGAAATGCAGCCGGCTCTGCCGCAGCTGCAGCAGCACGT |
| | | TCCGGTTCTGAAAGACTCTTCTCTGCTGTTCGAAGAATTCAAAAAAC |
| | | TGATCCGTAACCGTCAGTCTGAAGCTGCTGACTCTTCTCCGTCTGA |
| | | ACTGAAATACCTGGGTCTGGACACCCACTCTCGTAAAAAACGTCAG |
| | | CTGTACTCTGCTCTGGCTAACAAATGCTGCCACGTTGGTTGCACCA |
| | | AACGTTCTCTGGCTCGTTTCTGC |
| Relaxin2 | 80 | GACTCTTGGATGGAAGAAGTTATCAAACTGTGCGGTCGTGAACTGG |
| (XT100) | | TTCGTGCTCAGATCGCTATCTGCGGTATGTCTACCTGGTCTAAACG |
| | | 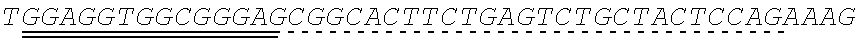 |
| | | 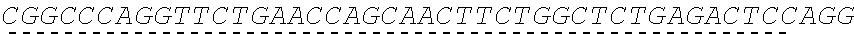 |
| | | 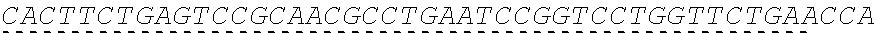 |
| | | 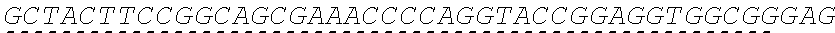 |
| | | 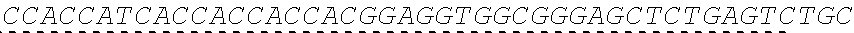 |
| | | 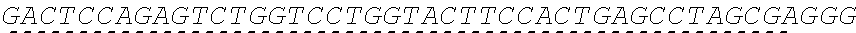 |
| | | 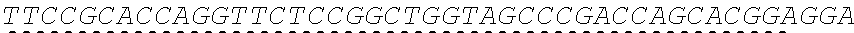 |
| | | 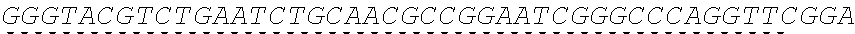 |
| | | 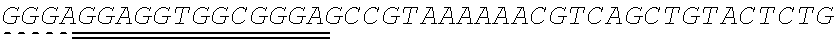 |
| | | CTCTGGCTAACAAATGCTGCCACGTTGGTTGCACCAAACGTTCTCT |
| | | GGCTCGTTTCTGC |
| Relaxin2 (XT35) | 81 | GACTCTTGGATGGAAGAAGTTATCAAACTGTGCGGTCGTGAACTGG |
| | | TTCGTGCTCAGATCGCTATCTGCGGTATGTCTACCTGGTCTAAACG |
| | | 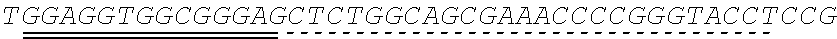 |
| | | 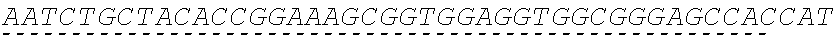 |
| | | 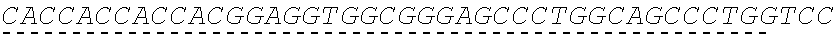 |
| | | 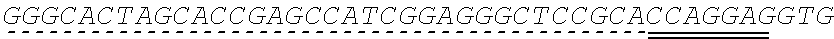 |
| | | 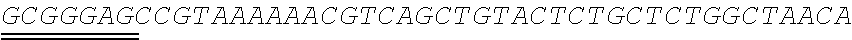 |
| | | AATGCTGCCACGTTGGTTGCACCAAACGTTCTCTGGCTCGTTTCTG |
| | | C |
| Relaxin2 | 82 | GACTCTTGGATGGAAGAAGTTATCAAACTGTGCGGTCGTGAACTGG |
| (single) | | 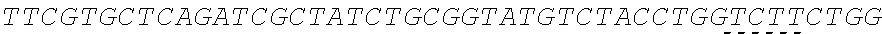 |
| | | 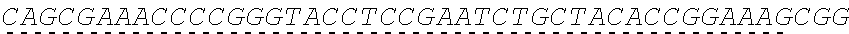 |
| | | 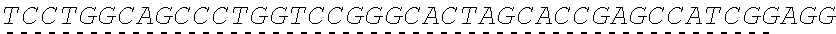 |
| | | 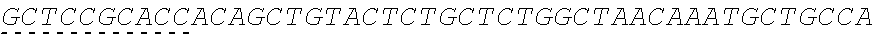 |
| | | CGTTGGTTGCACCAAACGTTCTCTGGCTCGTTTCTGC |
| Relaxin2 | 83 | GACTCTTGGATGGAAGAAGTTATCAAACTGTGCGGTCGTGAACTGG |
| (insulin C | | 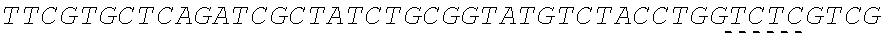 |
| peptide) | | 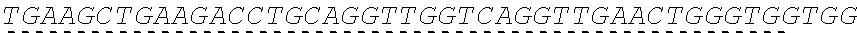 |
| | | 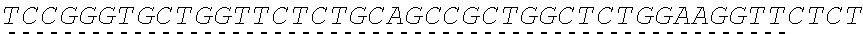 |
| | | 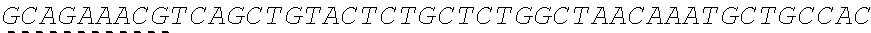 |
| | | GTTGGTTGCACCAAACGTTCTCTGGCTCGTTTCTGC |
| Relaxin2 (XT21) | 84 | GACTCTTGGATGGAAGAAGTTATCAAACTGTGCGGTCGTGAACTGG |
| | | TTCGTGCTCAGATCGCTATCTGCGGTATGTCTAC |
| | | 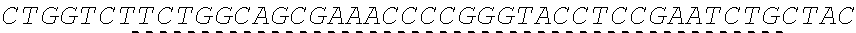 |
| | | 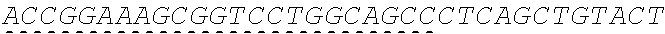 |
| | | CTGCTCTGGCTAACAAATGCTGCCACGTTGGTTGCACCAAACGTTC |
| | | TCTGGCTCGTTTCTGC |
| Insulin | 85 | TTTGTGAACCAACACCTGTGCGGCTCAGACCTGGTGGAAGCTCTCT |
| | | ACCTAGTGTGCGGGGAACGAGGCTTCTTCTACACAGACCCCACCG |
| | | GCGGAGGGCCCCGCCGGGGCATTGTGGAACAATGCTGTCACAGC |
| | | ATCTGCTCCCTCTACCAGCTGGAGAACTACTGCAAC |
| Oxyntomodulin | 86 | CATAGCCAGGGAACCTTCACCTCCGACTACAGCAAATACCTTGACA |
| | | GTAGGAGAGCTCAGGATTTTGTGCAATGGCTGATGAACACAAAGAG |
| | | GAATAAAAACAATATAGCC |
| GLP2 | 87 | CACGGCGACGGTTCATTCTCTGACGAAATGAATACAATACTCGACA |
| | | ACCTCGCCGCCAGGGACTTTATCAATTGGCTCATTCAAACTAAAAT |
| | | CACCGAC |
| Moka | 88 | ATCAACGTGAAGTGCAGCCTGCCCCAGCAGTGCATCAAGCCCTGC |
| | | AAGGACGCCGGCATGCGGTTCGGCAAGTGCATGAACAAGAAGTGC |
| | | AGGTGCTACAGC |
| Ssam6 | 89 | GCTGACAACAAATGCGAAAACTCTCTGCGTCGTGAAATCGCTTGCG |
| | | GTCAGTGCCGTGACAAAGTTAAAACCGACGGTTACTTCTACGAATG |
| | | CTGCACCTCTGACTCTACCTTCAAAAAATGCCAGGACCTGCTGCAC |
| 550 | 90 | GAATGCATCGGTATGTTCAAATCTTGCGACCCGGAAAACGACAAAT |
| | | GCTGCAAAGGTCGTACCTGCTCTCGTAAACACCGTTGGTGCAAATA |
| | | CAAACTG |
| Mambalign 1 | 91 | GAAGTTTTGCTATCATAACACTGGCATGCCTTTTCGAAATCTCAAGC |
| | | TCATCCTACAGGGATGTTCTTCTTCGTGCAGTGAAACAGAAAACAAT |
| | | AAGTGTTGCTCAACAGACAGATGCAACAAA |
| Relaxin2a | 92 | GACTCTTGGATGGAAGAAGTTATCAAACTGTGCGGTCGTGAACTGG |
| | | TTCGTGCTCAGATCGCTATCTGCGGTATGTCTACCTGGTCTAAACG |
| | | T |
| Relaxin2b | 93 | CGTAAAAAACGTCAGCTGTACTCTGCTCTGGCTAACAAATGCTGCC |
| | | ACGTTGGTTGCACCAAACGTTCTCTGGCTCGTTTCTGC |
| Glucagon | 94 | CATTCACAGGGCACATTCACCAGTGACTACAGCAAGTATCTGGACT |
| | | CCAGGCGTGCCCAAGATTTTGTGCAGTGGTTGATG |
| Relaxin2 (30GS) | 223 | GACTCTTGGATGGAAGAAGTTATCAAACTGTGCGGTCGTGAACTGG |
| | | 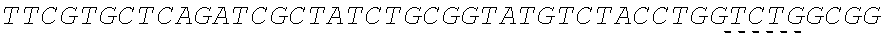 |
| | | 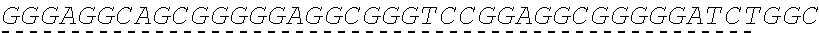 |
| | | 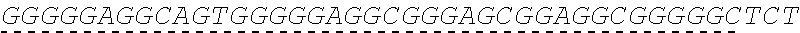 |
| | | CAGCTGTACTCTGCTCTGGCTAACAAATGCTGCCACGTTGGTTGCA |
| | | CCAAACGTTCTCTGGCTCGTTTCTGC |
| Relaxin2 Q60A | 224 | GACTCTTGGATGGAAGAAGTTATCAAACTGTGCGGTCGTGAACTGG |
| (30GS) | | 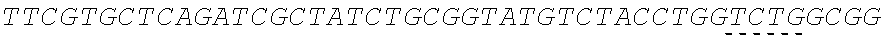 |
| | | 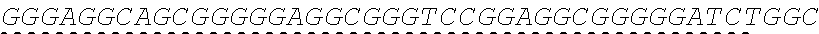 |
| | | 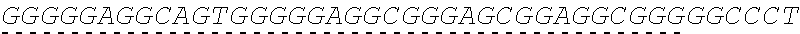 |
| | | GCGCTGTACTCTGCTCTGGCTAACAAATGCTGCCACGTTGGTTGCA |
| | | CCAAACGTTCTCTGGCTCGTTTCTGC |
| Relaxin2 (9GS) | 225 | GACTCTTGGATGGAAGAAGTTATCAAACTGTGCGGTCGTGAACTGG |
| | | 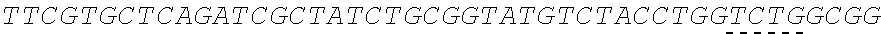 |
| | | 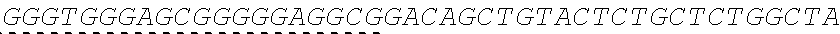 |
| | | ACAAATGCTGCCACGTTGGTTGCACCAAACGTTCTCTGGCTCGTTT |
| | | CTGC |
| Relaxin2c (9GS) | 226 | GATTCATGGATGGAGGAGGTCATCAAACTGTGTGGCAGGGAGCTG |
| | | 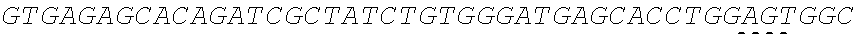 |
| | | 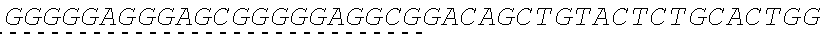 |
| | | CCAATAAATGCTGCCACGTGGGATGTACCAAGAGATCTCTGGCAC |
| | | GGTTTTGT |
| Relaxin2 | 227 | GACTCTTGGATGGAAGAAGTTATCAAACTGTGCGGTCGTGAACTGG |
| (GGGPRR) | | 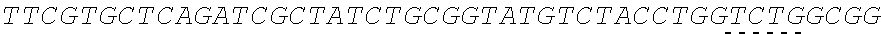 |
| | | 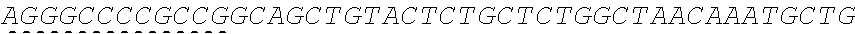 |
| | | CCACGTTGGTTGCACCAAACGTTCTCTGGCTCGTTTCTGC |
| Relaxin2 (18GS) | 228 | GACTCTTGGATGGAAGAAGTTATCAAACTGTGCGGTCGTGAACTGG |
| | | 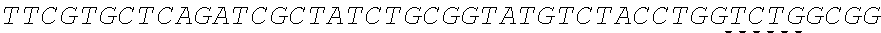 |
| | | 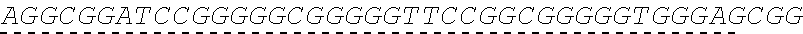 |
| | | 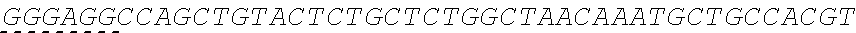 |
| | | TGGTTGCACCAAACGTTCTCTGGCTCGTTTCTGC |
| GLP1 | 229 | CATGGTGAAGGGACCTTTACCAGTGATGTAAGTTCTTATTTGGAAG |
| | | GCCAAGCTGCCAAGGAATTCATTGCTTGGCTGGTGAAA |

For SEQ ID NOs: 75-94, 223-229
Immunoglobulin Region = 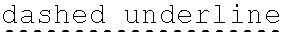 Peptide/Therapeutic peptide = italic
Peptide/Therapeutic peptide internal linker = 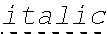 Connecting peptide = 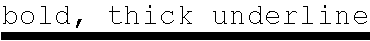 Extender peptide = 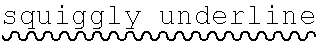 Linker = double underline
Protease site: underline

TABLE 6
Therapeutic Peptides—Amino Acid Sequence

| Name | SEQ ID NO | Sequence |
|---|---|---|
| Exendin-4 | 95 | HGEGTFTSDLSKQMEEEAVRLFIEWLKNGGPSSGAPPPS |
| Leptin | 96 | VPIQKVQDDTKTLIKTIVTRINDISHTQSVSSKQKVTGLDFIPGLHPILT |
| | | LSKMDQTLAVYQQILTSMPSRNVIQISNDLENLRDLLHVLAFSKSCHLP |
| | | WASGLETLDSLGGVLEASGYSTEVVALSRLQGSLQDMLWQLDLSPGC |
| ZP1 | 97 | HSQGTFTSDYSKYLDSKAAHDFVEWLLRA |
| ZPCEX | 98 | HSQGTFTSDYSKYLDSKAAHDFVEWLLNGGPSSGAPPPS |
| Relaxin2 | 99 | DSWMEEVIKLCGRELVRAQIAICGMSTWSKRSLSQEDAPQTPRPVAEI |
| | | VPSFINKDTETINMMSEFVANLPQELKLTLSEMQPALPQLQQHVPVL |
| | | KDSSLLFEEFKKLIRNRQSEAADSSPSELKYLGLDTHSRKKRQLYSALA |
| | | NKCCHVGCTKRSLARFC |
| Relaxin2 | 100 | 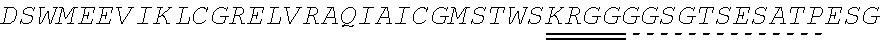 |
| (XT100) | | 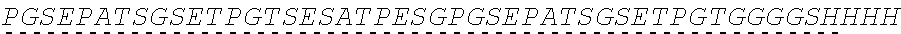 |
| | | 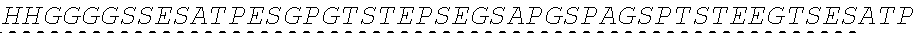 |
| | | 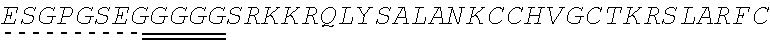 |
| Relaxin2 (XT35) | 101 | 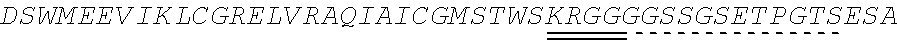 |
| | | 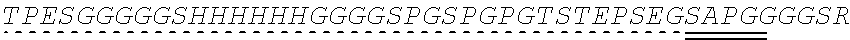 |
| | | KKRQLYSALANKCCHVGCTKRSLARFC |
| Relaxin2 | 102 | 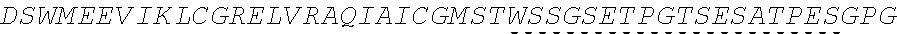 |
| (single) | | 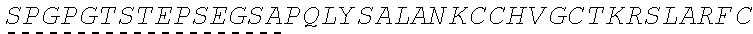 |
| Relaxin2 | 103 | 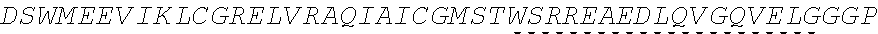 |
| (insulin C | | 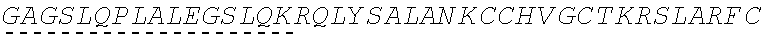 |
| peptide) | | |
| Relaxin2 (XT21) | 104 | 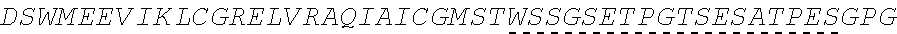 |
| | | 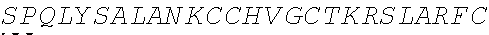 |
| Insulin | 105 | FVNQHLCGSDLVEALYLVCGERGFFYTDPTGGGPRRGIVEQCCHSIC |
| | | SLYQLENYCN |
| Oxyntomodulin | 106 | HSQGTFTSDYSKYLDSRRAQDFVQWLMNTKRNKNNIA |
| GLP2 | 107 | HGDGSFSDENINTILDNLAARDFINWLIQTKITD |
| Moka | 108 | INVKCSLPQQCIKPCKDAGMRFGKCMNKKCRCYS |
| Ssam6 | 109 | ADNKCENSLRREIACGQCRDKVKTDGYFYECCTSDSTFKKCQDLLH |
| 550 | 110 | ECIGMFKSCDPENDKCCKGRTCSRKHRWCKYKL |
| Mambalign 1 | 111 | LKCYQHGKVVTCHRDMKFCYHNTGMPFRNLKLILQGCSSSCSETEN |
| | | NKCCSTDRCNK |
| Relaxin2a | 112 | DSWMEEVIKLCGRELVRAQIAICGMSTWSKR |
| Relaxin2b | 113 | RKKRQLYSALANKCCHVGCTKRSLARFC |
| Glucagon | 114 | HSQGTFTSDYSKYLDSRRAQDFVQWLM |
| Relaxin2 (30GS) | 230 | 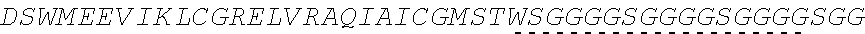 |
| | | 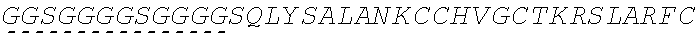 |
| Relaxin2 Q60A | 231 | 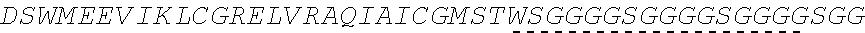 |
| (30GS) | | 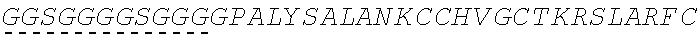 |
| Relaxin2 (9GS) | 232 | 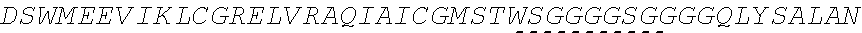 |
| | | KCCHVGCTKRSLARFC |
| Relaxin2c (9GS) | 233 | 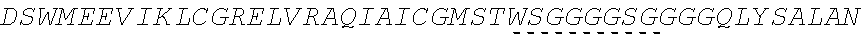 |
| | | KCCHVGCTKRSLARFC |
| Relaxin2 | 234 | 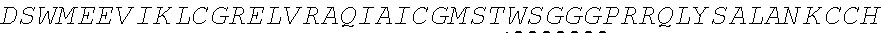 |
| (GGGPRR) | | VGCTKRSLARFC |
| Relaxin2 (18GS) | 235 | 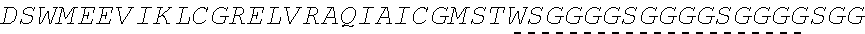 |
| | | 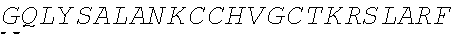 |
| GLP1 | 236 | HGEGTFTSDVSSYLEGQAAKEFIAWLVK |

For SEQ ID NOs: 95-114, 230-236
Immunoglobulin Region = 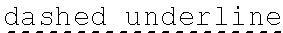 Peptide/Therapeutic peptide = italic
Peptide/Therapeutic peptide internal linker = 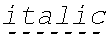 Connecting peptide = 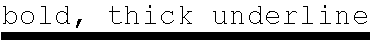 Extender peptide = 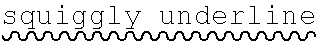 Linker = double underline
Protease site: underline

TABLE 7
Connecting Peptide Sequences

| Name | SEQ ID NO | Sequence |
|---|---|---|
| (GGGGS)ₙ | 115 | GGGGSₙ₌₁₋₁₀ |
| (GGGGG)ₙ | 116 | GGGGGₙ₌₁₋₁₀ |
| CEXa | 117 | NGGPSSGAPPPSGGGGG |
| CEXb | 118 | GGPSSGAPPPSGGGGG |
| EAAAK | 237 | EAAAKEAAAKEAAAK |
| CEXGGGGS | 238 | GGPSSGAPPPSGGGGS |
| XT21 | 239 | SGSETPGTSESATPESGPGSP |

TABLE 8
Extender Peptide Sequences

| Name | SEQ ID NO | Sequence |
|---|---|---|
| Extender a | 119 | 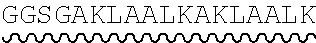 |
| Extender b | 120 | 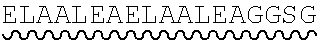 |

TABLE 9
Linker Sequences

| Name | SEQ ID NO | Sequence |
|---|---|---|
| Linker a | 121 | GGGGG |
| Linker b | 122 | GGGGS |

TABLE 10
Internal Linker Sequences

| Name | SEQ ID NO | Sequence |
|---|---|---|
| XT100 | 123 | 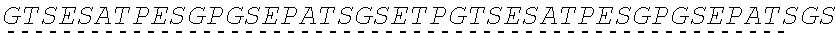 |
| | | 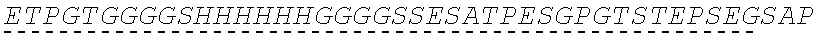 |
| | | 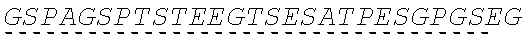 |
| XT35 | 124 | 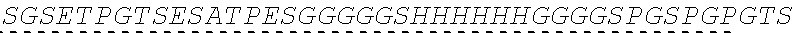 |
| | | 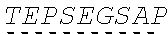 |
| Insulin C peptide | 125 | 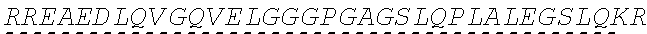 |
| XT21 | 126 | 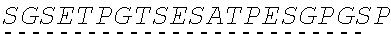 |
| XT35 (noHIS) | 240 | 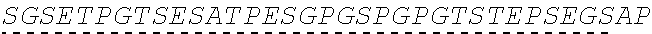 |
| 30GS | 241 | 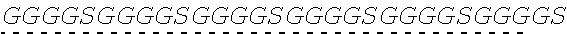 |
| 9GS | 242 | 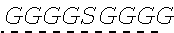 |
| 18GS | 243 | 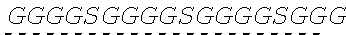 |
| GGGPRR | 244 | 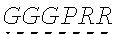 |

TABLE 11
Peptides not derived from an immunoglobulin—Nucleic acid sequence

| Name | SEQ ID NO | Sequence |
|---|---|---|
| Exendin-4 | 127 | CACGGAGAAGGAACATTTACCAGCGACCTCAGCAAGCAGAT |
| | | GGAGGAAGAGGCCGTGAGGCTGTTCATCGAGTGGCTGAAGA |
| | | ACGGCGGACCCTCCTCTGGCGCTCCACCCCCTAGC |
| Leptin | 128 | GTTCCAATTCAAAAGGTTCAAGATGATACCAAAACTCTGATTA |
| | | AAACTATTGTCACGCGTATAAACGACATCTCACATACCCAGTC |
| | | GGTTAGCTCAAAGCAAAAAGTTACCGGTTTGGACTTTATTCCG |
| | | GGACTGCACCCGATCCTGACCCTTAGTAAAATGGACCAGACA |
| | | CTGGCCGTCTACCAGCAAATCCTGACATCGATGCCATCCAGA |
| | | AATGTGATACAAATTAGCAACGATTTGGAAAACCTTCGCGATC |
| | | TGCTGCACGTGCTGGCCTTCAGTAAGTCCTGTCATCTGCCGT |
| | | GGGCGTCGGGACTGGAGACTCTTGACTCGCTGGGTGGAGTG |
| | | TTAGAGGCCTCTGGCTATTCTACTGAAGTCGTTGCGCTGTCA |
| | | CGCCTCCAGGGGAGCCTGCAGGACATGCTGTGGCAGCTGG |
| | | ACCTGTCACCTGGCTGC |
| ZP1 | 129 | CACAGCCAGGGCACATTCACTAGCGATTATAGTAAATATCTG |
| | | GATTCCAAGGCAGCGCACGATTTTGTAGAGTGGCTCTTGC |
| ZPCEX | 130 | CACAGCCAGGGCACATTCACTAGCGATTATAGTAAATATCTG |
| | | GATTCCAAGGCAGCGCACGATTTTGTAGAGTGGCTCTTGAAC |
| | | GGAGGCCCTTCCTCCGGAGCTCCACCTCCGTCC |
| Relaxin2 | 131 | GACTCTTGGATGGAAGAAGTTATCAAACTGTGCGGTCGTGAA |
| | | CTGGTTCGTGCTCAGATCGCTATCTGCGGTATGTCTACCTGG |
| | | TCTAAACGTTCTCTGTCTCAGGAAGACGCTCCGCAGACCCCG |
| | | CGTCCGGTTGCTGAAATCGTTCCGTCTTTCATCAACAAAGAC |
| | | ACCGAAACCATCAACATGATGTCTGAATTCGTTGCTAACCTGC |
| | | CGCAGGAACTGAAACTGACCCTGTCTGAAATGCAGCCGGCT |
| | | CTGCCGCAGCTGCAGCAGCACGTTCCGGTTCTGAAAGACTC |
| | | TTCTCTGCTGTTCGAAGAATTCAAAAAACTGATCCGTAACCGT |
| | | CAGTCTGAAGCTGCTGACTCTTCTCCGTCTGAACTGAAATAC |
| | | CTGGGTCTGGACACCCACTCTCGTAAAAAACGTCAGCTGTAC |
| | | TCTGCTCTGGCTAACAAATGCTGCCACGTTGGTTGCACCAAA |
| | | CGTTCTCTGGCTCGTTTCTGC |
| Relaxin2 (XT100) | 132 | GACTCTTGGATGGAAGAAGTTATCAAACTGTGCGGTCGTGAA |
| | | CTGGTTCGTGCTCAGATCGCTATCTGCGGTATGTCTACCTGG |
| | | 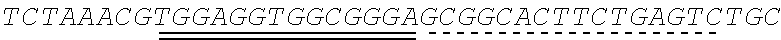 |
| | | 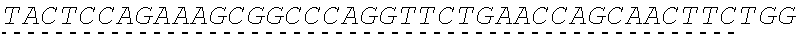 |
| | | 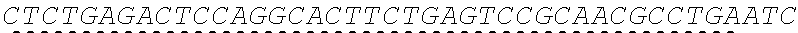 |
| | | 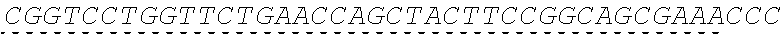 |
| | | 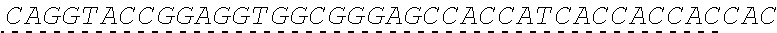 |
| | | 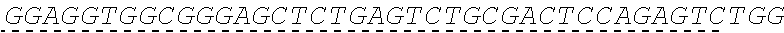 |
| | | 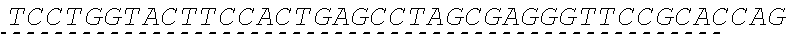 |
| | | 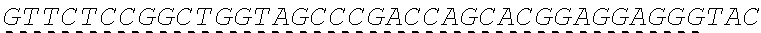 |
| | | 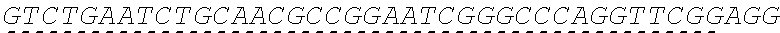 |
| | | 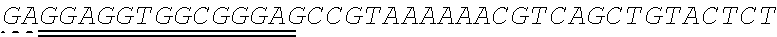 |
| | | GCTCTGGCTAACAAATGCTGCCACGTTGGTTGCACCAAACGT |
| | | TCTCTGGCTCGTTTCTGC |
| Relaxin2 (XT35) | 133 | GACTCTTGGATGGAAGAAGTTATCAAACTGTGCGGTCGTGAA |
| | | CTGGTTCGTGCTCAGATCGCTATCTGCGGTATGTCTACCTGG |
| | | 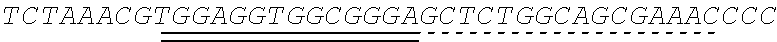 |
| | | 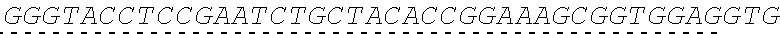 |
| | | 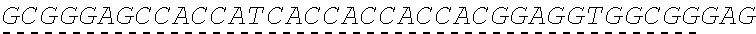 |
| | | 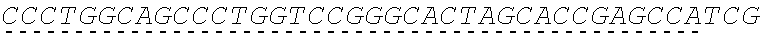 |
| | | 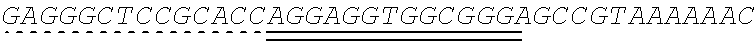 |
| | | GTCAGCTGTACTCTGCTCTGGCTAACAAATGCTGCCACGTTG |
| | | GTTGCACCAAACGTTCTCTGGCTCGTTTCTGC |
| Relaxin2 (single) | 134 | GACTCTTGGATGGAAGAAGTTATCAAACTGTGCGGTCGTGAA |
| | | CTGGTTCGTGCTCAGATCGCTATCTGCGGTATGTCTACCTGG |
| | | 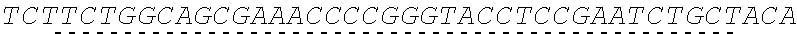 |
| | | 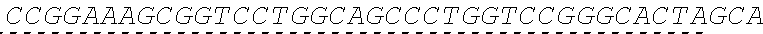 |
| | | 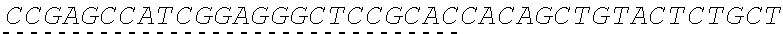 |
| | | CTGGCTAACAAATGCTGCCACGTTGGTTGCACCAAACGTTCT |
| | | CTGGCTCGTTTCTGC |
| Relaxin2 (insulin C | 135 | GACTCTTGGATGGAAGAAGTTATCAAACTGTGCGGTCGTGAA |
| peptide) | | CTGGTTCGTGCTCAGATCGCTATCTGCGGTATGTCTACCTGG |
| | | 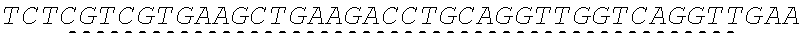 |
| | | 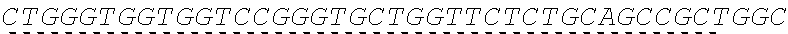 |
| | | 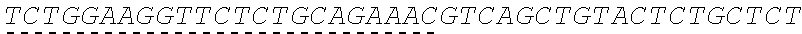 |
| | | GGCTAACAAATGCTGCCACGTTGGTTGCACCAAACGTTCTCT |
| | | GGCTCGTTTCTGC |
| Relaxin2 (XT21) | 136 | GACTCTTGGATGGAAGAAGTTATCAAACTGTGCGGTCGTGAA |
| | | CTGGTTCGTGCTCAGATCGCTATCTGCGGTATGTCTAC |
| | | 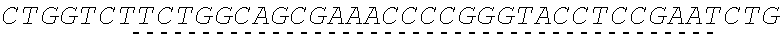 |
| | | 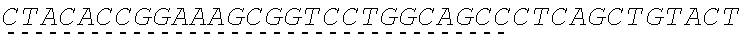 |
| | | CTGCTCTGGCTAACAAATGCTGCCACGTTGGTTGCACCAAAC |
| | | GTTCTCTGGCTCGTTTCTGC |
| Insulin | 137 | TTTGTGAACCAACACCTGTGCGGCTCAGACCTGGTGGAAGCT |
| | | CTCTACCTAGTGTGCGGGGAACGAGGCTTCTTCTACACAGAC |
| | | CCCACCGGCGGAGGGCCCCGCCGGGGCATTGTGGAACAAT |
| | | GCTGTCACAGCATCTGCTCCCTCTACCAGCTGGAGAACTACT |
| | | GCAAC |
| Oxyntomodulin | 138 | CATAGCCAGGGAACCTTCACCTCCGACTACAGCAAATACCTT |
| | | GACAGTAGGAGAGCTCAGGATTTTGTGCAATGGCTGATGAAC |
| | | ACAAAGAGGAATAAAAACAATATAGCC |
| GLP2 | 139 | CACGGCGACGGTTCATTCTCTGACGAAATGAATACAATACTC |
| | | GACAACCTCGCCGCCAGGGACTTTATCAATTGGCTCATTCAA |
| | | ACTAAAATCACCGAC |
| Moka | 140 | ATCAACGTGAAGTGCAGCCTGCCCCAGCAGTGCATCAAGCC |
| | | CTGCAAGGACGCCGGCATGCGGTTCGGCAAGTGCATGAACA |
| | | AGAAGTGCAGGTGCTACAGC |
| Ssam6 | 141 | GCTGACAACAAATGCGAAAACTCTCTGCGTCGTGAAATCGCT |
| | | TGCGGTCAGTGCCGTGACAAAGTTAAAACCGACGGTTACTTC |
| | | TACGAATGCTGCACCTCTGACTCTACCTTCAAAAAATGCCAG |
| | | GACCTGCTGCAC |
| 550 | 142 | GAATGCATCGGTATGTTCAAATCTTGCGACCCGGAAAACGAC |
| | | AAATGCTGCAAAGGTCGTACCTGCTCTCGTAAACACCGTTGG |
| | | TGCAAATACAAACTG |
| Mambalign 1 | 143 | GAAGTTTTGCTATCATAACACTGGCATGCCTTTTCGAAATCTC |
| | | AAGCTCATCCTACAGGGATGTTCTTCTTCGTGCAGTGAAACA |
| | | GAAAACAATAAGTGTTGCTCAACAGACAGATGCAACAAA |
| Relaxin2a | 245 | GACTCTTGGATGGAAGAAGTTATCAAACTGTGCGGTCGTGAA |
| | | CTGGTTCGTGCTCAGATCGCTATCTGCGGTATGTCTACCTGG |
| | | TCTAAACGT |
| Relaxin2b | 246 | CGTAAAAAACGTCAGCTGTACTCTGCTCTGGCTAACAAATGC |
| | | TGCCACGTTGGTTGCACCAAACGTTCTCTGGCTCGTTTCTGC |
| Glucagon | 247 | CATTCACAGGGCACATTCACCAGTGACTACAGCAAGTATCTG |
| | | GACTCCAGGCGTGCCCAAGATTTTGTGCAGTGGTTGATG |
| Relaxin2 (30GS) | 248 | GACTCTTGGATGGAAGAAGTTATCAAACTGTGCGGTCGTGAA |
| | | CTGGTTCGTGCTCAGATCGCTATCTGCGGTATGTCTACCTGG |
| | | 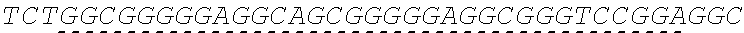 |
| | | 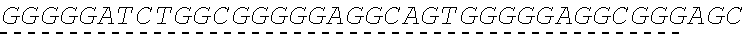 |
| | | 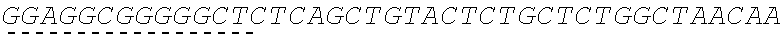 |
| | | ATGCTGCCACGTTGGTTGCACCAAACGTTCTCTGGCTCGTTT |
| | | CTGC |
| Relaxin2 Q60A | 249 | GACTCTTGGATGGAAGAAGTTATCAAACTGTGCGGTCGTGAA |
| (30GS) | | CTGGTTCGTGCTCAGATCGCTATCTGCGGTATGTCTACCTGG |
| | | 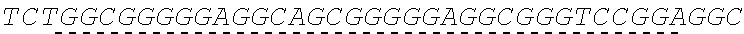 |
| | | 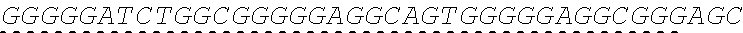 |
| | | 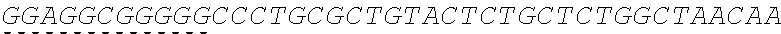 |
| | | ATGCTGCCACGTTGGTTGCACCAAACGTTCTCTGGCTCGTTT |
| | | CTGC |
| Relaxin2 (9GS) | 250 | GACTCTTGGATGGAAGAAGTTATCAAACTGTGCGGTCGTGAA |
| | | CTGGTTCGTGCTCAGATCGCTATCTGCGGTATGTCTACCTGG |
| | | 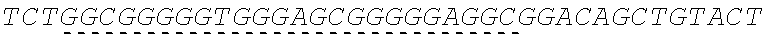 |
| | | CTGCTCTGGCTAACAAATGCTGCCACGTTGGTTGCACCAAAC |
| | | GTTCTCTGGCTCGTTTCTGC |
| Relaxin2c (9GS) | 251 | GATTCATGGATGGAGGAGGTCATCAAACTGTGTGGCAGGGA |
| | | GCTGGTGAGAGCACAGATCGCTATCTGTGGGATGAGCACCT |
| | | 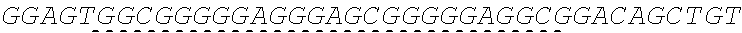 |
| | | ACTCTGCACTGGCCAATAAATGCTGCCACGTGGGATGTACCA |
| | | AGAGATCTCTGGCACGGTTTTGT |
| Relaxin2 | 252 | GACTCTTGGATGGAAGAAGTTATCAAACTGTGCGGTCGTGAA |
| (GGGPRR) | | CTGGTTCGTGCTCAGATCGCTATCTGCGGTATGTCTACCTGG |
| | | 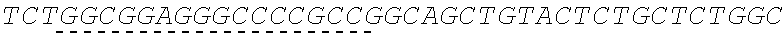 |
| | | TAACAAATGCTGCCACGTTGGTTGCACCAAACGTTCTCTGGC |
| | | TCGTTTCTGC |
| Relaxin2 (18GS) | 253 | GACTCTTGGATGGAAGAAGTTATCAAACTGTGCGGTCGTGAA |
| | | CTGGTTCGTGCTCAGATCGCTATCTGCGGTATGTCTACCTGG |
| | | 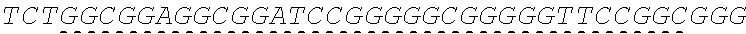 |
| | | 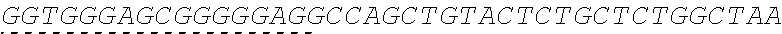 |
| | | CAAATGCTGCCACGTTGGTTGCACCAAACGTTCTCTGGCTCG |
| | | TTTCTGC |
| GLP1 | 254 | CATGGTGAAGGGACCTTTACCAGTGATGTAAGTTCTTATTTGG |
| | | AAGGCCAAGCTGCCAAGGAATTCATTGCTTGGCTGGTGAAA |

TABLE 12
Peptides not derived from an immunoglobulin—Amino acid sequences

| Name | SEQ ID NO | Sequence |
|---|---|---|
| Exendin-4 | 144 | HGEGTFTSDLSKQMEEEAVRLFIEWLKNGGPSSGAPPPS |
| Leptin | 145 | VPIQKVQDDTKTLIKTIVTRINDISHTQSVSSKQKVTGLDFIPGL |
| | | HPILTLSKMDQTLAVYQQILTSMPSRNVIQISNDLENLRDLLHVL |
| | | AFSKSCHLPWASGLETLDSLGGVLEASGYSTEVVALSRLQGSLQ |
| | | DMLWQLDLSPGC |
| ZP1 | 146 | HSQGTFTSDYSKYLDSKAAHDFVEWLLRA |
| ZPCEX | 147 | HSQGTFTSDYSKYLDSKAAHDFVEWLLNGGPSSGAPPPS |
| Relaxin2 | 148 | DSWMEEVIKLCGRELVRAQIAICGMSTWSKRSLSQEDAPQTPRP |
| | | VAEIVPSFINKDTETINMMSEFVANLPQELKLTLSEMQPALPQL |
| | | QQHVPVLKDSSLLFEEFKKLIRNRQSEAADSSPSELKYLGLDTH |
| | | SRKKRQLYSALANKCCHVGCTKRSLARFC |
| Relaxin2 (XT100) | 149 | 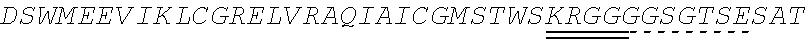 |
| | | 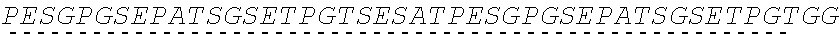 |
| | | 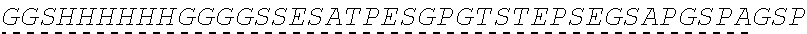 |
| | | 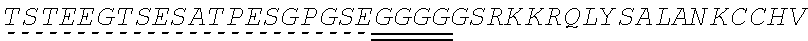 |
| | | GCTKRSLARFC |
| Relaxin2 (XT35) | 150 | 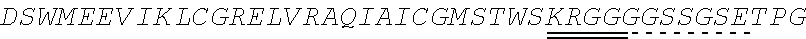 |
| | | 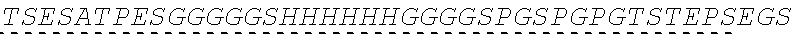 |
| | | 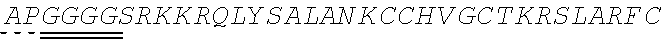 |
| Relaxin2 (single) | 151 | 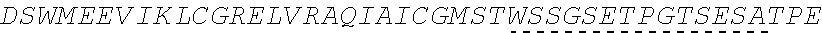 |
| | | 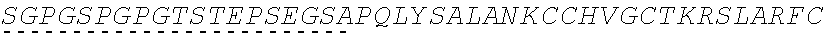 |
| Relaxin2 (insulin C | 152 | 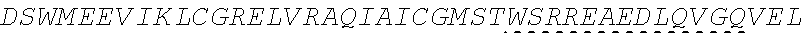 |
| peptide) | | 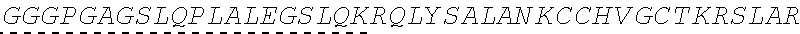 |
| | | FC |
| Relaxin2 (XT21) | 153 | 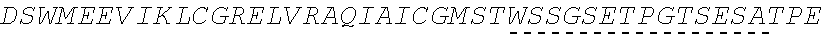 |
| | | 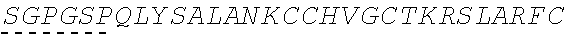 |
| Insulin | 154 | FVNQHLCGSDLVEALYLVCGERGFFYTDPTGGGPRRGIVEQCC |
| | | HSICSLYQLENYCN |
| Oxyntomodulin | 155 | HSQGTFTSDYSKYLDSRRAQDFVQWLMNTKRNKNNIA |
| GLP2 | 156 | HGDGSFSDEMNTILDNLAARDFINWLIQTKITD |
| Moka | 157 | INVKCSLPQQCIKPCKDAGMRFGKCMNKKCRCYS |
| Ssam6 | 158 | ADNKCENSLRREIACGQCRDKVKTDGYFYECCTSDSTFKKCQD |
| | | LLH |
| 550 | 159 | ECIGMFKSCDPENDKCCKGRTCSRKHRWCKYKL |
| Mambalign 1 | 160 | LKCYQHGKVVTCHRDMKFCYHNTGMPFRNLKLILQGCSSSCS |
| | | ETENNKCCSTDRCNK |
| Relaxin2a | 255 | DSWMEEVIKLCGRELVRAQIAICGMSTWSKR |
| Relaxin2b | 256 | RKKRQLYSALANKCCHVGCTKRSLARFC |
| Glucagon | 257 | HSQGTFTSDYSKYLDSRRAQDFVQWLIVI |
| Relaxin2 (30GS) | 258 | 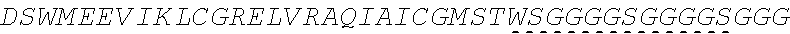 |
| | | 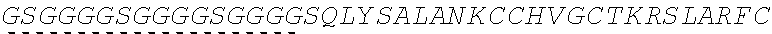 |
| Relaxin2 Q60A | 259 | 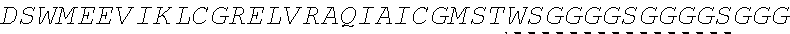 |
| (30GS) | | 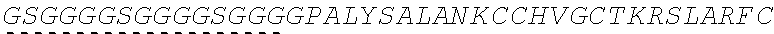 |
| Relaxin2 (9GS) | 260 | 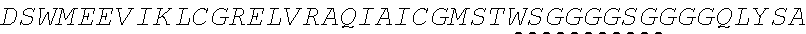 |
| | | LANKCCHVGCTKRSLARFC |
| Relaxin2c (9GS) | 261 | 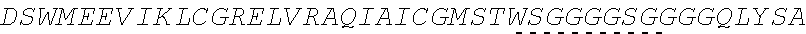 |
| | | LANKCCHVGCTKRSLARFC |
| Relaxin2 | 262 | 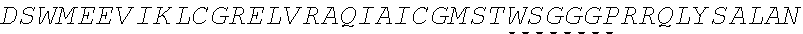 |
| (GGGPRR) | | KCCHVGCTKRSLARFC |
| Relaxin2 (18GS) | 263 | 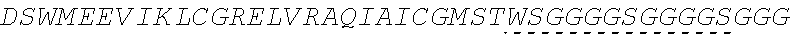 |
| | | 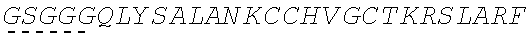 |
| GLP1 | 264 | HGEGTFTSDVSSYLEGQAAKEFIAWLVK |

1-73. (canceled)

74. A composition comprising: (a) a non-immunoglobulin region comprising a therapeutic peptide, (b) a connecting peptide, and (c) a first immunoglobulin region comprising a variable region; wherein the non-immunoglobulin region is connected to the amino-terminus of the first immunoglobulin region with the connecting peptide.

75. The composition of claim 74, wherein the non-immunoglobulin region does not comprise more than 10 consecutive amino acids from an immunoglobulin.

76. The composition of claim 74, wherein the therapeutic peptide comprises a hormone or a toxin.

77. The composition of claim 76, wherein the hormone is selected from a glucagon-like peptide-1 receptor agonist and a member of the insulin superfamily.

78. The composition of claim 76, wherein the hormone is selected from relaxin, exendin-4, glucagon-like peptide-1, glucagon-like peptide-2, oxyntomodulin, leptin, betatrophin, bovine growth hormone, human growth hormone, erythropoietin (EPO), parathyroid hormone, and somatostatin.

79. The composition of claim 76, wherein the toxin is selected from Mokatoxin-1, VM2, Protoxin-2, ziconotide, chlorotoxin, neurotoxin mu-SLPTX-Ssm6a (Ssam6), kappa-theraphotoxin-Tb1a (550 peptide), and mambalign-1.

80. The composition of claim 74, wherein the therapeutic peptide is configured to treat one or more diseases or conditions when administered to a subject in need thereof.

81. The composition of claim 80, wherein the one or more diseases or conditions is selected from heart failure, a heart failure related condition, fibrosis, a fibrosis related condition, diabetes, a diabetes related condition, obesity, an obesity related condition, short bowel syndrome, a short bowel syndrome related condition, inflammatory bowel disease, an inflammatory bowel disease related condition, autoimmune disease, an autoimmune disease related condition, and pain.

82. The composition of claim 74, wherein the therapeutic peptide comprises relaxin A chain, relaxin B chain, or a combination of relaxin A chain and relaxin B chain.

83. The composition of claim 82, comprising relaxin A chain and relaxin B chain connected by a peptide or a disulfide bond.

84. The composition of claim 74, wherein the therapeutic peptide comprises a first therapeutic region, an internal linker, and a second therapeutic region.

85. The composition of claim 74, wherein the non-immunoglobulin region comprises an amino acid sequence selected from SEQ ID NOs: 144-160 and 255-264.

86. The composition of claim 74, wherein the immunoglobulin region comprises a fragment crystallizable (Fc) region.

87. The composition of claim 74, wherein the immunoglobulin region comprises an amino acid sequence comprising 20 or more consecutive amino acids of an immunoglobulin selected from SEQ ID NOs: 5-8.

88. The composition of claim 74, comprising a second immunoglobulin region connected to the first immunoglobulin region by a peptide or a disulfide bond.

89. The composition of claim 88, wherein the first immunoglobulin region is an immunoglobulin heavy chain and the second immunoglobulin region is an immunoglobulin light chain, or the first immunoglobulin region is an immunoglobulin light chain and the second immunoglobulin region is an immunoglobulin heavy chain.

90. A method of treating a disease or condition in a subject in need thereof with a non-immunoglobulin therapeutic peptide, the method comprising administering to the subject an immunoglobulin fusion comprising the therapeutic peptide connected to the amino-terminus of a variable region of an immunoglobulin region, wherein the non-immunoglobulin therapeutic peptide does not comprise more than 10 consecutive amino acids from an immunoglobulin.

91. The method of claim 90, wherein the therapeutic peptide is selected from human GCSF, bovine GCSF, Mokatoxin-1, Vm24, mambalign-1, kappa-theraphotoxin-Tb1a (550 peptide), glucagon-like peptide-1, exendin-4, erythropoietin (EPO), FGF21, GMCSF, human interferon-beta, human interferon-alpha, relaxin, Protoxin-2, oxyntomodulin, leptin, betatrophin, growth differentiation factor 11 (GDF11), parathyroid hormone, angiopoietin-like 3 (ANGPTL3), IL-11, human growth hormone (hGH), BCCX2, elafin, ZP1, ZPCEX, relaxin, insulin, glucagon-like peptide-2, neurotoxin mu-SLPTX-Ssm6a (Ssam6), and glucagon.

92. The method of claim 91, wherein the therapeutic peptide comprises a relaxin peptide.

93. The method of claim 92, wherein the disease or condition comprises: heart failure, acute coronary syndrome, atrial fibrillation, cardiac fibrosis, coronary artery disease, ischemia reperfusion associated with solid organ transplant, cardiopulmonary bypass, ischemic stroke, corneal healing, diabetic nephropathy, cirrhosis, portal hypertension, diabetic would healing, systemic sclerosis, cervical ripening at time of labor, preeclampsia, portal hypertension, fibrosis, and combinations thereof.
